(19) United States
(12) Patent Application Publication
(10) Pub. No.: US 2023/0024096 A1
(43) Pub. Date: Jan. 26, 2023

(54) BIFUNCTIONAL COMPOUNDS FOR THE TREATMENT OF CANCER

(71) Applicants: Hoffmann-La Roche Inc., Little Falls, NJ (US); C4 THERAPEUTICS, INC., Watertown, MA (US)

(72) Inventors: Martin DUPLESSIS, Watertown, MA (US); Delphine GAUFRETEAU, Basel (CH); Roman HUTTER, Basel (CH); Eleonora JOVCHEVA, Basel (CH); Bernd KUHN, Basel (CH); Kiel LAZARSKI, Watertown, MA (US); Yanke LIANG, Watertown, MA (US); Thomas LUEBBERS, Basel (CH); Laetitia Janine MARTIN, Basel (CH); Rainer E. MARTIN, Basel (CH); Barbara Johanna MUELLER, Basel (CH); Roger NORCROSS, Basel (CH); Philipp SCHMID, Basel (CH); Jean-Yves WACH, Basel (CH)

(21) Appl. No.: 17/771,204

(22) PCT Filed: Oct. 28, 2020
(86) PCT No.: PCT/EP2020/080265

§ 371 Date: Apr. 22, 2022

Related U.S. Application Data
(60) Provisional application No. 62/927,340, filed on Oct. 29, 2019.

(30) Foreign Application Priority Data
Nov. 15, 2019 (EP) 19209344.1

(51) Int. Cl. A61K 47/55; A61K 47/54; A61K 45/06

(52) U.S. Cl. CPC A61K 47/55; A61K 47/545; A61K 45/06

(57) ABSTRACT

The invention provides bifunctional compounds of formula (I) or a pharmaceutically acceptable salt thereof. Formula (I). The compounds cause the degradation of SMARCA2 via the targeted ubiquination of SMARCA2 protein and subsequent proteasomal degradation and are thus useful for the treatment of cancer. The targeting ligand is of formula (TL).

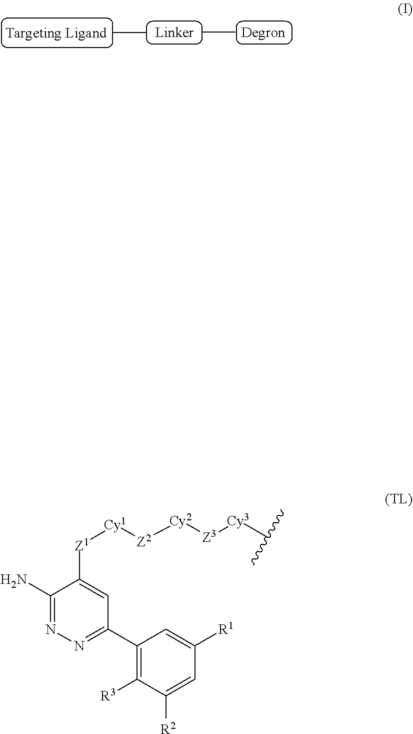

### FIELD OF THE INVENTION

The present invention relates novel bifunctional compounds, which function to recruit targeted proteins to E3 Ubiquitin Ligase for degradation, and methods of preparation and uses thereof. More specifically, the compounds of the present invention cause the degradation of SMARCA2 via the targeted ubiquitination of SMARCA2 protein and subsequent proteasomal degradation. The present compounds are thus useful for the treatment or prophylaxis of abnormal cellular proliferation, including tumors and cancer.

### BACKGROUND OF THE INVENTION

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and therefore, are more attractive therapeutic targets than general proteasome inhibitors due to their specificity for certain protein substrates. The development of ligands of E3 ligases has proven challenging, in part due to the fact that they must disrupt protein-protein interactions. However, recent developments have provided specific ligands which bind to these ligases. For example, since the discovery of nutlins, the first small molecule E3 ligase mouse double minute 2 homolog (MDM2) inhibitors, additional compounds have been reported that target MDM2 (i.e, human double minute 2 or HDM2) E3 ligases (J. Di, et al. Current Cancer Drug Targets (2011), 11(8), 987-994).

One E3 ligase with exciting therapeutic potential is cereblon (CRBN). CRBN is known as primary target for anticancer thalidomide analogs. A higher expression of CRBN has been linked to the efficiency of thalidomide analogs in cancer therapy.

The field of targeted protein degradation promoted by small molecules has been intensively studied over the last years (e.g. Collins et al., Biochem J, 2017, 474(7), 1127-47). Bifunctional compounds, such as those that are described in U.S. Patent Application Publications 2016-0235730, function to recruit endogenous proteins to an E3 ubiquitin ligase for degradation.

The Switch/Sucrose Non Fermentable (SWI/SNF) is a multi-subunit complex that modulates chromatic structure through the activity of two mutually exclusive helicase/ATPase catalytic subunits: SWI/SNF-Related, Matrix-Associated, Actin-Dependent Regulator of Chromatin, Subfamily A, Member 2 (SMARCA2, BRAHMA or BRM) and SWI/SNF-Related, Matrix-Associated, Actin-Dependent Regulator of Chromatin, Subfamily A, Member 4 (SMARCA4 or BRG1). The core and the regulatory subunits couple ATP hydrolysis to the perturbation of histone-DNA contacts, thereby providing access points to transcription factors and cognate DNA elements that facilitate gene activation and repression.

Mutations in the genes encoding the twenty canonical SWI/SNF subunits are observed in nearly 20% of all cancers with the highest frenquency of mutations observed in rhabdoid tumors, female cancers (including ovarian, uterine, cerical and endometrial), lung adenocarcinoma, gastric adenocarcinoma, melanoma, esophageal, and renal clear cell carcinoma. Despite having a high degree of homology, and their presumed overlapping functions, SMARCA2 and SMARCA4 have been reported as having different roles in cancer. For example, SMARCA4 is frequently mutated in primary tumors, while SMARCA2 inactivation is infrequent in tumor development. In fact, numerous types of cancer have been shown to be SMARCA4-related (e.g., cancers having a SMARCA4-mutation or a SMARCA4-deficiency, such as lack of expression), including, e.g., lung cancer (such as non-small cell lung cancer).

SMARCA2 has been demonstrated as one of the top essential genes in SMARCA4-related or -mutant cancer cell lines. This is because SMARCA4-deficient patient populations or cells depend exclusively on SMARCA2 activity—i.e., there is a greater incorporation of SMARCA2 into the complex to compensate for the SMARCA4 deficiency. Thus, SMARCA2 may be targeted in SMARCA4-related/deficient cancers. The co-occurrence of the deficiency of the expression of two (or more) genes that leads to cell death is known as synthetic lethality. Accordingly, synthetic lethality can be leveraged in the treatment of certain SMARCA2/SMARCA4-related cancers.

There is an ongoing need for effective treatment for diseases that are treatable by inhibiting or degrading SMARCA2 (i.e., BRAHMA or BRM). However, non-specific effects, and the inability to target and modulate SMARCA2 remains an obstacle to the development of effective treatments. As such, small-molecule therapeutic agents that target SMARCA2 and that leverage or potentiate CRBN's substrate specificity would be very useful.

### SUMMARY OF THE INVENTION

The present invention provides a bifunctional compound of formula (I)

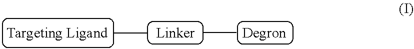

or a pharmaceutically acceptable salt thereof, wherein said Targeting Ligand, Linker and Degron are as described herein.

In a further aspect, the present invention provides compounds of formula (I) as defined herein, or pharmaceutically acceptable salts thereof, for use as therapeutically active substance.

In a further aspect, the present invention provides pharmaceutical compositions comprising a compound of formula (I) as defined herein, or a pharmaceutically acceptable salt thereof, and a therapeutically inert carrier.

In a further aspect, the present invention provides a compound of formula (I) as defined herein, or a pharmaceutically acceptable salt thereof, for use in the treatment of SMARCA2-mediated disorders, in particular cancer.

### DETAILED DESCRIPTION OF THE INVENTION

The present invention provides compounds of formula I and pharmaceutically acceptable salts thereof, the preparation of the above mentioned compounds, medicaments containing them and their manufacture as well as the use of the above mentioned compounds in the therapeutic and/or prophylactic treatment of cancer.

### Definitions

The following definitions of the general terms used in the present description apply irrespectively of whether the terms in question appear alone or in combination with other groups.

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

The term “Targeting Ligand” (or target protein moiety or target protein ligand or ligand) as used herein refers to a small molecule of formula (TL) as defined herein, which is capable of binding to or binds to a target protein of interest, such as to SMARCA2.

The term “Linker” as used herein refers to a chemical moiety selected from formulae L-1 to L-23 as define herein that serves to link a Targeting Ligand with a Degron.

The Degron is a compound that serves to link a targeted protein, through the Linker and Targeting Ligand, to a ubiquitin ligase for proteosomal degradation. In certain embodiments, the Degron is a compound that is capable of binding to or binds to a ubiquitin ligase. In further embodiments, the Degron is a compound that is capable of binding to or binds to a E3 Ubiquitin Ligase. In further embodiments, the Degron is a compound that is capable of binding to or binds to cereblon. In further embodiments, the Degron is a thalidomide or a derivative or analog thereof.

The term “cereblon” or “CRBN” as used herein refers to the ubiquitously expressed E3 ligase protein cereblon. Cereblon is a protein that forms an E3 ubiquitin ligase complex, which ubiquinates various other proteins. Cereblon is known as primary target for anticancer thalidomide analogs. A higher expression of cereblon has been linked to the efficiency of thalidomide analogs in cancer therapy.

The term “alkyl”, alone or in combination with other groups, stands for a hydrocarbon radical which may be linear or branched, with single or multiple branching, wherein the alkyl group in general comprises 1 to 6 carbon atoms (C₁₋₆-alkyl), for example, methyl (Me), ethyl (Et), propyl, isopropyl (i-propyl), n-butyl, i-butyl (isobutyl), 2-butyl (sec-butyl), t-butyl (tert-butyl), isopentyl, 2-ethyl-propyl (2-methyl-propyl), 1,2-dimethyl-propyl and the like. A specific group is methyl.

The term “alkyldiyl” as used herein refers to a saturated linear or branched-chain divalent hydrocarbon radical of about one to six carbon atoms (C₁-C₆). Examples of alkyldiyl groups include, but are not limited to, methylene (—CH₂—), ethylene (—CH₂CH₂—), propylene (—CH₂CH₂CH₂—), and the like. An alkyldiyl group may also be referred to as an “alkylene” group.

The term “haloalkyl”, alone or in combination with other groups, refers to alkyl as defined herein, which is substituted by one or multiple halogen, particularly 1-5 halogen, more particularly 1-3 halogen. Particular halogen is fluoro. Examples include 2,2,2-trifluoroethyl, trifluoromethyl, difluoromethyl, fluoromethyl and the like.

The term “haloalkoxy”, alone or in combination with other groups, refers to alkoxy as defined herein, which is substituted by one or multiple halogen, particularly 1-5 halogen, more particularly 1-3 halogen. Particular halogen is fluoro. Examples include 2,2,2-trifluoroethoxy, trifluoromethoxy, difluoromethoxy, fluoromethoxy and the like.

The term “aminoalkyl”, alone or in combination with other groups, refers to alkyl as defined herein, which is substituted by one or multiple amino groups, particularly 1-5 amino groups, more particularly 1-3 amino groups. Examples include 2-aminoethyl, aminomethyl, and the like.

The term “cycloalkyl” denotes a monovalent saturated monocyclic or bicyclic hydrocarbon group of 3 to 10 ring carbon atoms, particularly a monovalent saturated monocyclic hydrocarbon group of 3 to 8 ring carbon atoms. Bicyclic means consisting of two carbocycles having one or more carbon atoms in common, while one carbocycle is saturated, the other one may be aromatic. Particular cycloalkyl groups are monocyclic. Examples for monocyclic cycloalkyl are “C₃₋₇cycloalkyl” such as cyclopropyl, cyclobutanyl, cyclopentyl, cyclohexyl or cycloheptyl. Examples for saturated bicyclic cycloalkyl are bicyclo[2.2.1]heptanyl, or bicyclo[2.2.2]octanyl. Examples for bicyclic cycloalkyl wherein one ring is aromatic are 1H-indenyl or 1,2,3,4-tetrahydronaphthalenyl.

The term “hydroxy”, alone or in combination with other groups, refers to OH.

The term “amino”, alone or in combination with other groups, refers to NH₂.

The term “cyano”, alone or in combination with other groups, refers to CN (i.e. nitrile).

The term “carbonyl”, alone or in combination with other groups, refers to C(═O).

The term “halogen”, alone or in combination with other groups, denotes chloro (Cl), iodo (I), fluoro (F) and bromo (Br). A specific group is F.

The term “heteroaryl” denotes a monovalent heterocyclic mono- or bicyclic ring system of 5 to 14 ring atoms, comprising 1, 2, 3 or 4 heteroatoms selected from N, O and S, the remaining ring atoms being carbon and in which at least one ring is aromatic. Examples of heteroaryl moieties include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, azepinyl, diazepinyl, isoxazolyl, benzofuranyl, isothiazolyl, benzothienyl, indolinyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, quinoxalinyl, or 2,3-dihydropyrrolo[2,3-b]pyridinyl. Specific examples include benzimidazolyl, pyridinyl, thiazolyl, indolinyl, 1,2,3,4-tetrahydroquinolinyl, 3,4-dihydroquinolinyl, benzofuranyl, furanyl, imidazolyl, isoindolyl, and quinolinyl.

The term “heterocyclyl” denotes a monovalent saturated or partly unsaturated mono- or bicyclic ring system of 3 to 14 ring atoms, comprising 1, 2, or 3 ring heteroatoms selected from N, O and S, the remaining ring atoms being carbon. Examples for monocyclic saturated heterocyclyl include azetidinyl, pyrrolidinyl, tetrahydrofuranyl, tetrahydrothienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocyclyl include 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocyclyl include dihydrofuryl, imidazolinyl, dihydro-oxazolyl, tetrahydro-pyridinyl, or dihydropyranyl. Specific examples include piperazinyl, piperidinyl, pyrrolidinyl and 3,8-diazabicyclo[3.2.1]octanyl.

The term “heterocyclyloxy”, alone or in combination with other groups, stands for an —O-heterocyclyl radical, for example, pyrrolidinyloxy, piperidyloxy, morpholinyloxy and the like.

The term “alkoxy”, alone or in combination with other groups, stands for an —O—C₁₋₆-alkyl radical which may be linear or branched, with single or multiple branching, wherein the alkyl group in general comprises 1 to 6 carbon atoms (C₁₋₆-alkoxy), for example, methoxy (OMe, MeO), ethoxy (OEt), propoxy, isopropoxy (i-propoxy), n-butoxy, i-butoxy (iso-butoxy), 2-butoxy (sec-butoxy), t-butoxy (tert-butoxy), isopentyloxy (i-pentyloxy) and the like. Particular “C₁₋₆-alkoxy” are groups with 1 to 4 carbon atoms. A specific group is methoxy.

The term “aryl” denotes a monovalent aromatic carbocyclic mono- or bicyclic ring system comprising 6 to 10 carbon ring atoms. Examples of aryl moieties include phenyl (Ph), and naphthyl. Specific “aryl” is phenyl.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

The term “a pharmaceutically acceptable salt” refers to a salt that is suitable for use in contact with the tissues of humans and animals. Examples of suitable salts with inorganic and organic acids are, but are not limited to acetic acid, citric acid, formic acid, fumaric acid, hydrochloric acid, lactic acid, maleic acid, malic acid, methane-sulfonic acid, nitric acid, phosphoric acid, p-toluenesulphonic acid, succinic acid, sulfuric acid (sulphuric acid), tartaric acid, trifluoroacetic acid and the like. Particular acids are formic acid, trifluoroacetic acid and hydrochloric acid. Specific acids are hydrochloric acid, trifluoroacetic acid and fumaric acid.

The term “as defined herein” and “as described herein” when referring to a variable incorporates by reference the broad definition of the variable as well as particularly, more particularly and most particularly definitions, if any.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “aromatic” denotes the conventional idea of aromaticity as defined in the literature, in particular in IUPAC—Compendium of Chemical Terminology, 2^{nd} Edition, A. D. McNaught & A. Wilkinson (Eds). Blackwell Scientific Publications, Oxford (1997).

The term “therapeutically inert carrier” denotes any ingredient having no therapeutic activity and being non-toxic such as disintegrators, binders, fillers, solvents, buffers, tonicity agents, stabilizers, antioxidants, surfactants or lubricants used in formulating pharmaceutical products.

Whenever a chiral carbon is present in a chemical structure, it is intended that all stereoisomers associated with that chiral carbon are encompassed by the structure as pure stereoisomers as well as mixtures thereof.

All separate embodiments may be combined.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “cancer” refers to a disease characterized by the presence of a neoplasm or tumor resulting from abnormal uncontrolled growth of cells (such cells being “cancer cells”). As used herein, the term cancer explicitly includes, but is not limited to, hepatocellular cancer, malignancies and hyperproliferative disorders of the colon (colon cancer), lung cancer, breast cancer, prostate cancer, melanoma, and ovarian cancer.

### Compounds of the Invention

In a first aspect (A1), the present invention provides a compound of formula (I)

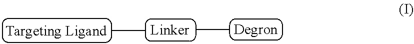

or a pharmaceutically acceptable salt thereof, wherein:
said targeting ligand is of formula (TL):

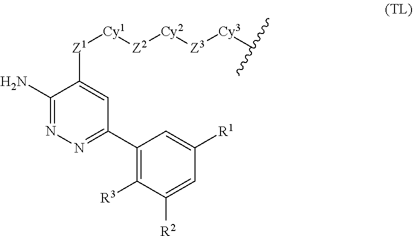

wherein:
R¹ and R² are each independently selected from the group consisting of hydrogen and halogen;
R³ is selected from the group consisting of amino and hydroxy;
Z¹ is:
   (i) absent;
   (ii) —O—; or
   (iii) —O—C₁-C₆-alkyldiyl-CH(C₆-C₁₀-aryl)-C₁-C₆-alkyldiyl-NHC(O)—; Cy¹ is 3-14 membered heterocyclyl optionally substituted with 1-3 substituents R⁴;
Z² is:
   (i) absent;
   (ii) carbonyl;
   (iii) —NH—;
   (iv) —C₁-C₆-alkyldiyl-;
   (v) —C₁-C₆-alkyldiyl-NH—;
   (vi) —O(CH₂)ₐ;
   (vii) —C(O)NH(CH₂)_{b}—;
   (viii) —(CH₂)_{c}NHC(O)(CH₂)_{d}X¹—;
   (ix) —O—C₁-C₆-alkyldiyl-C(O)—;
   (x) —O—C₁-C₆-alkyldiyl-C(O)NH—; or
   (xi) —CH₂N(C₁-C₆-alkyl)CH₂—;
Cy² is:
   (i) absent;
   (ii) C₆-C₁₀-aryl optionally substituted with 1-3 substituents R⁵;
   (iii) C₃-C₁₀-cycloalkyl optionally substituted with 1-3 substituents R⁶;
   (iv) 3-14 membered heterocyclyl optionally substituted with 1-3 substituents R⁷; or
   (v) 5-14 membered heteroaryl optionally substituted with 1-3 substituents R⁸.
Z³ is:
   (i) absent;
   (ii) —X²(CH₂)ₑ—; or
   (iii) —(CH₂)ₑX²;
Cy³ is:
   (i) absent;
   (ii) C₆-C₁₀-aryl optionally substituted with 1-3 substituents R⁹;
   (iii) C₃-C₁₀-cycloalkyl optionally substituted with 1-3 substituents R¹⁰; or
   (iv) 3-14 membered heterocyclyl optionally substituted with 1-3 substituents R¹¹;
a, b, c, d, and e are each independently an integer selected from 0, 1, 2, 3, 4, 5, and 6;
X¹ is:
   (i) absent;
   (ii) —NH—; or
   (iii) —O—;
X² is:
   (i) absent;
   (ii) carbonyl;
   (iii) —O—; or
   (iv) —NHC(O)—;
R⁴, R⁵, R⁶, R⁷, R⁸, R⁹, R¹⁰, and R¹¹ are each independently selected from the group consisting of hydroxy, amino, cyano, halogen, C₁-C₆-alkyl, C₁-C₆-alkoxy, halo-C₁-C₆-alkyl, halo-C₁-C₆-alkoxy, amino-C₁-C₆-alkyl, (C₁-C₆-alkyl)₂N—C₁-C₆-alkyl-, (C₁-C₆-alkyl)₂N—C₁-C₆-alkoxy-, C₁-C₆-alkyl-NH—C₁-C₆-alkyl-, C₁-C₆-alkyl-NH—C(O)—, C₁-C₆-alkyl-C(O)—NH—, 3-14 membered heterocyclyl, 3-14 membered heterocyclyloxy, 3-14 membered heterocyclyl-C₁-C₆-alkyl, 3-14 membered heterocyclyl-C₁-C₆-alkoxy and C₆-C₁₀-aryl; and
the wavy line indicates the point of attachment to the linker;
said linker is a covalent bond or is selected from the group consisting of formulae L-1 to L-23;

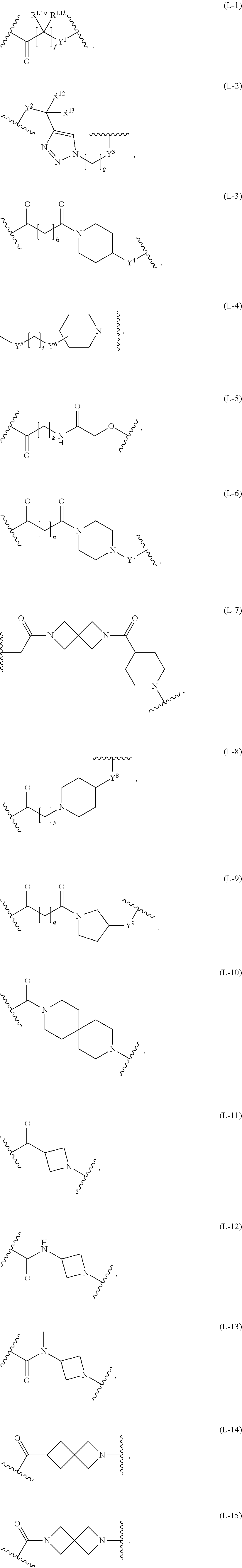

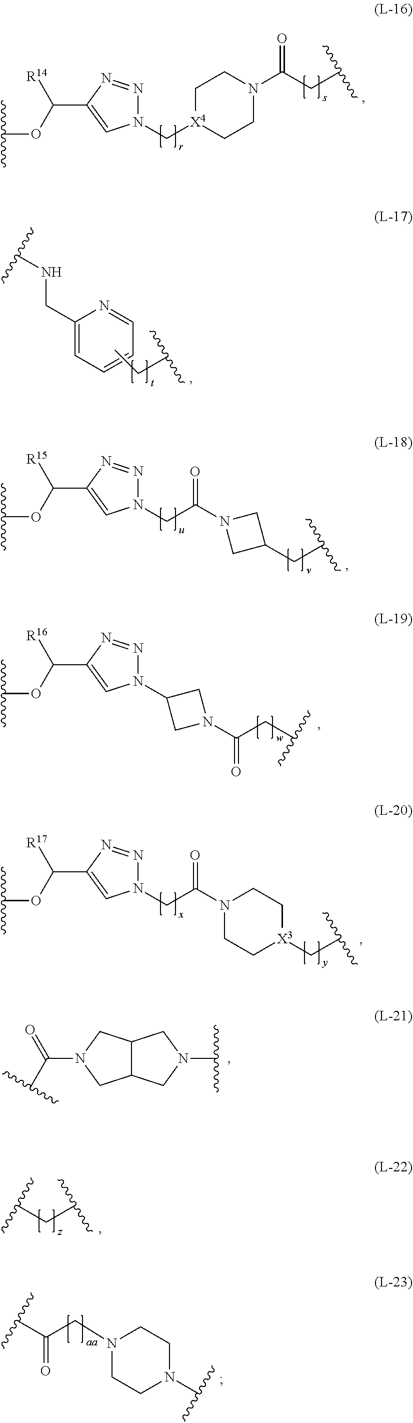

wherein.
X³ and X⁴ are independently selected from the group consisting of CH and N;
R¹² and R¹³ are independently selected from the group consisting of hydrogen and C₁-C₆-alkyl; or
R² and R¹³, taken together with the carbon atom to which they are attached, form a C₃-C₁₀-cycloalkyl ring;
R¹⁴, R¹⁵, R¹⁶, and R¹⁷ are independently selected from the group consisting of hydrogen and C₁-C₆-alkyl;
R^{L1a} and R^{L1b} are each independently selected from the group consisting of hydrogen, C₁-C₆-alkyl, halo-C₁-C₆-alkyl, C₁-C₆-alkoxy, halo-C₁-C₆-alkoxy, C₃-C₁₀-cycloalkyl, 3-14 membered heterocyclyl, C₆-C₁₀-aryl and 5-14 membered heteroaryl;
f, g, h, i, k, m, n, p, q, r, s, t, u, v, w, x, y, z, and aa are each independently an integer selected from 0, 1, 2, 3, 4, 5, 6, 7, 8, 9, 10, 11, 12, 13, and 14;
Y¹, Y², Y³, Y⁴, Y⁵, Y⁶, Y⁷, Y⁸, and Y⁹ are each independently absent or selected from the group consisting of —O—, —NH—, —N(C₁-C₆-alkyl)-, —C₁-C₆-alkyldiyl-, —NH—C₁-C₆-alkyldiyl-, —O—C₁-C₆-alkyldiyl-, carbonyl, —NHC(O)—, —N(C₁-C₆-alkyl)-C(O)—, —C(O)—N(C₁-C₆-alkyl)-, and —C(O)NH—;
each occurrence of a wavy line indicates the point of attachment of the linker to the targeting ligand or to the degron; and
said degron is selected from the group consisting of formulae (DG-1), (DG-2), (DG-3) and (DG-4):

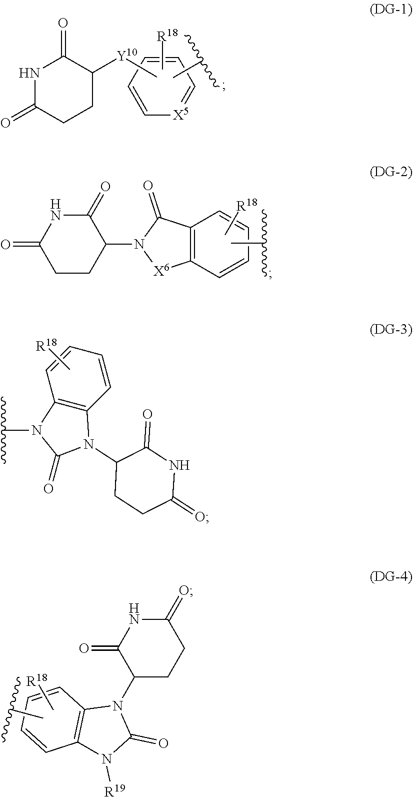

wherein:
X⁵ is CH or N;
X⁶ is CH₂ or C(O);
each R¹⁸ is independently selected from the group consisting of hydrogen, halogen and C₁-C₆-alkyl;
R¹⁹ is selected from the group consisting of hydrogen and C₁-C₆-alkyl;
Y¹⁰ is a covalent bond, —O— or —NR—, wherein R is selected from the group consisting of hydrogen, C₁-C₆-alkyl, halo-C₁-C₆-alkyl, C₃-C₁₀-cycloalkyl, 3-14 membered heterocyclyl, C₆-C₁₀-aryl and 5-14 membered heteroaryl; and the wavy line indicates the point of attachment to the linker.

In one embodiment, the present invention provides a compound of formula (I) as described herein, or a pharmaceutically acceptable salt thereof, wherein said targeting ligand is of formula (TL), wherein:
R¹ and R² are each independently selected from the group consisting of hydrogen and halogen;
R³ is selected from the group consisting of amino and hydroxy;
Z¹ is:
   (i) absent;
   (ii) —O—; or
   (iii) —O—C₁-C₆-alkyldiyl-CH(C₆-C₁₀-aryl)-C₁-C₆-alkyldiyl-NHC(O)—;
Cy¹ is 3-14 membered heterocyclyl optionally substituted with R⁴;
Z² is:
   (i) absent;
   (ii) carbonyl;
   (iii) —O(CH₂)ₐ—;
   (iv) —C(O)NH(CH₂)_{b}—;
   (v) —(CH₂)_{c}NHC(O)(CH₂)_{d}X¹—;
   (vi) —O—C₁-C₆-alkyldiyl-C(O)NH—; or
   (vii) —CH₂N(C₁-C₆-alkyl)CH₂—;
Cy² is:
   (i) absent;
   (ii) C₆-C₁₀-aryl optionally substituted with R⁵;
   (iii) C₃-C₁₀-cycloalkyl;
   (iv) 3-14 membered heterocyclyl; or
   (v) 5-14 membered heteroaryl;
Z³ is:
   (i) absent;
   (ii) —X²(CH₂)ₑ—; or
   (iii) —(CH₂)ₑX²;
Cy³ is:
   (i) absent;
   (ii) C₆-C₁₀-aryl;
   (iii) C₃-C₁₀-cycloalkyl; or
   (iv) 3-14 membered heterocyclyl;
a is 0, 1 or 2;
b is 0 or 1;
c, d, and e are each independently an integer selected from 0, 1, 2 and 3;
X¹ is:
   (i) absent;
   (ii) —NH—; or
   (iii) —O—;
X² is:
   (i) absent;
   (ii) carbonyl;
   (iii) —O—; or
   (iv) —NHC(O)—;
R⁴ is C₆-C₁₀-aryl;
R⁵ is selected from the group consisting of halogen, C₁-C₆-alkyl, and halo-C₁-C₆-alkyl; and
the wavy line indicates the point of attachment to the linker.

In a preferred embodiment, the present invention provides a compound of formula (I) as described herein, or a pharmaceutically acceptable salt thereof, wherein said targeting ligand is of formula (TL), wherein:
R¹ is selected from the group consisting of hydrogen and halogen;
R² is hydrogen;
R³ is hydroxy;
Z¹ is absent;
Cy¹ is 3-14 membered heterocyclyl optionally substituted with R⁴;
Z² is:
   (i) absent;
   (ii) carbonyl; or
   (iii) —C(O)NHCH₂—;
Cy² is:
   (i) C₆-C₁₀-aryl;
   (ii) 3-14 membered heterocyclyl; or
   (iii) 5-14 membered heteroaryl;
Z³ is —X²(CH₂)ₑ—;
Cy³ is 3-14 membered heterocyclyl;
e is an integer selected from 0, 1 and 2;
X² is:
   (i) absent; or
   (ii) —O—;
R⁴ is C₆-C₁₀-aryl; and
the wavy line indicates the point of attachment to the linker.

In a particularly preferred embodiment, the present invention provides a compound of formula (I) as described herein, or a pharmaceutically acceptable salt thereof, wherein said targeting ligand is of formula (TL), wherein:
R¹ is selected from the group consisting of hydrogen and fluoro;
R² is hydrogen;
R³ is hydroxy;
Z¹ is absent;
Cy¹ is selected from the group consisting of:

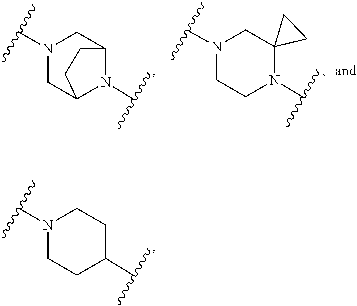 [SMILES: *N1CC2CCC(C1)N2* ; *N1CCN(*)C2(CC2)C1 ; *C1CCN(*)CC1]

optionally substituted with R⁴; wherein each wavy line indicates the point of attachment to Z² or to the remainder of formula (TL);
Z² is:
   (i) absent;
   (ii) carbonyl; or
   (iii) —C(O)NHCH₂—;
Cy² is
   (i) phenyl;
   (ii) 3-14 membered heterocyclyl selected from:

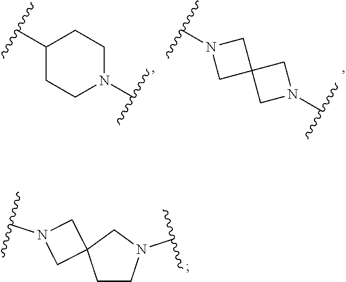 [SMILES: *C1CCN(*)CC1 ; *N1CC2(C1)CN(*)C2 ; *N1CCC2(C1)CN(*)C2]

wherein each wavy line indicates the point of attachment to Z² or Z³; or
(iii) pyrimidinyl;
Z³ is —X²(CH₂)ₑ—;
Cy³ is 3-14 membered heterocyclyl selected from:

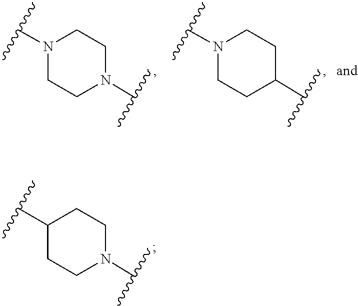 [SMILES: *N1CCN(*)CC1 ; *C1CCN(*)CC1 ; *C1CCN(*)CC1]

wherein each wavy line indicates the point of attachment to Z³ or the linker;
e is an integer selected from 0, 1 and 2;
X² is:
   (i) absent; or
   (ii) —O—;
R⁴ is phenyl; and
the wavy line indicates the point of attachment to the linker.

In one embodiment, the present invention provides a compound of formula (I) as described herein, or a pharmaceutically acceptable salt thereof, wherein said linker is a covalent bond or is selected from the group consisting of formulae L-1 to L-23, wherein:
X³ and X⁴ are independently selected from the group consisting of CH and N;
R¹² and R¹³ are independently selected from the group consisting of hydrogen and C₁-C₆-alkyl; or
R¹² and R¹³, taken together with the carbon atom to which they are attached, form a C₃-C₁₀-cycloalkyl ring;
R¹⁴ is hydrogen or C₁-C₆-alkyl;
R¹⁵ is hydrogen;
R¹⁶ is C₁-C₆-alkyl;
R¹⁷ is hydrogen;
f is an integer selected from 1, 2, 5, 6, 7, 8, 9
g is an integer selected from 3, 6, 8, 9, 10, 11, 14
h is 2,
i is an integer selected from 0, 1, 2, 3,
k is 3;
m is 1;
n is an integer selected from 8 and 12;
p is an integer selected from 0, 1, and 8;
q is 7;
r is an integer selected from 0 and 1;
s is 4;
t is 9;
u is 4;
v is 1;
w is 4;
x is an integer selected from 2 and 4;
y is an integer selected from 1 and 3;
z is 1;
aa is an integer selected from 0, 1, and 8;
Y¹ is —O— or —NH—;
Y² is —O—, —NH—, —C₁-C₆-alkyldiyl- or —NH—C₁-C₆-alkyldiyl-;
Y³ is absent, —O—C₁-C₆-alkyldiyl- or carbonyl;
Y⁴ is —O—, —NH—, —N(C₁-C₆-alkyl)- or —C₁-C₆-alkyldiyl-;
Y⁵ is absent or carbonyl;
Y⁶ is absent, carbonyl, —O—, —NHC(O)—, —C(O)—N(C₁-C₆-alkyl)- or —C(O)NH—;
Y⁷ is absent or —C₁-C₆-alkyldiyl-;
Y⁸ is absent or —O—;
Y⁹ is —NH—; and
each occurrence of a wavy line indicates the point of attachment of the linker to the targeting ligand or to the degron.

In a preferred embodiment, the present invention provides a compound of formula (I) as described herein, or a pharmaceutically acceptable salt thereof, wherein said linker is a covalent bond or is selected from the group consisting of formulae L-4, L-8, L-13, and L-23, wherein:
i is an integer selected from 0, 2, and 3;
p is an integer selected from 0 and 1;
aa is 0;
Y⁵ is absent;
Y⁶ is absent, carbonyl, —O— or —C(O)—N(C₁-C₆-alkyl)-;
Y⁸ is absent or —O—; and
each occurrence of a wavy line indicates the point of attachment of the linker to the targeting ligand or to the degron.

In a particularly preferred embodiment, the present invention provides a compound of formula (I) as described herein, or a pharmaceutically acceptable salt thereof, wherein said linker is a covalent bond or is selected from the group consisting of formulae L-4, L-8, L-13, and L-23, wherein:
i is an integer selected from 0, 2, and 3;
p is an integer selected from 0 and 1;
aa is 0;
Y⁵ is absent;
Y⁶ is absent, carbonyl, —O— or —C(O)—NCH₃—;
Y is absent or —O—; and
each occurrence of a wavy line indicates the point of attachment of the linker to the targeting ligand or to the degron.

In one embodiment, the present invention provides a compound of formula (I) as described herein, or a pharmaceutically acceptable salt thereof, wherein said degron is selected from the group consisting of formulae (DG-1) and (DG-2), wherein:
X⁵ is CH or N;
X⁶ is CH₂ or C(O);
R¹⁸ is hydrogen;
Y¹⁰ is a covalent bond, —O— or —NH—; and
the wavy line indicates the point of attachment to the linker.

In a preferred embodiment, the present invention provides a compound of formula (I) as described herein, or a pharmaceutically acceptable salt thereof, wherein said degron is selected from the group consisting of formulae (DG-1) and (DG-2), wherein:
X⁵ is CH;
X⁶ is C(O);
R¹⁸ is hydrogen;
Y¹⁰ is —NH—; and
the wavy line indicates the point of attachment to the linker.

In one embodiment, the present invention provides a compound of formula (I) as described herein, or a pharmaceutically acceptable salt thereof, wherein said targeting ligand is of formula (TL), wherein:
R¹ and R² are each independently selected from the group consisting of hydrogen and halogen;
R³ is selected from the group consisting of amino and hydroxy;
Z¹ is:
   (i) absent;
   (ii) —O—; or
   (iii) —O—C₁-C₆-alkyldiyl-CH(C₆-C₁₀-aryl)-C₁-C₆-alkyldiyl-NHC(O)—;
Cy¹ is 3-14 membered heterocyclyl optionally substituted with R⁴;
Z² is:
   (i) absent;
   (ii) carbonyl;
   (iii) —O(CH₂)ₐ;
   (iv) —C(O)NH(CH₂)_{b}—;
   (v) —(CH₂)_{c}NHC(O)(CH₂)_{d}X¹—;
   (vi) —O—C₁-C₆-alkyldiyl-C(O)NH—; or
   (vii) —CH₂N(C₁-C₆-alkyl)CH₂—;
Cy² is:
   (i) absent;
   (ii) C₆-C₁₀-aryl optionally substituted with R⁵;
   (iii) C₃-C₁₀-cycloalkyl;
   (iv) 3-14 membered heterocyclyl; or
   (v) 5-14 membered heteroaryl;
Z³ is:
   (i) absent;
   (ii) —X²(CH₂)ₑ—; or
   (iii) —(CH₂)ₑX²;
Cy³ is:
   (i) absent;
   (ii) C₆-C₁₀-aryl;
   (iii) C₃-C₁₀-cycloalkyl; or
   (iv) 3-14 membered heterocyclyl;
a is 0, 1 or 2;
b is 0 or 1;
c, d, and e are each independently an integer selected from 0, 1, 2 and 3;
X¹ is:
   (i) absent;
   (ii) —NH—; or
   (iii) —O—;
X² is:
   (i) absent;
   (ii) carbonyl;
   (iii) —O—; or
   (iv) —NHC(O)—;
R⁴ is C₆-C₁₀-aryl;
R⁵ is selected from the group consisting of halogen, C₁-C₆-alkyl, and halo-C₁-C₆-alkyl; and
the wavy line indicates the point of attachment to the linker;
wherein said linker is a covalent bond or is selected from the group consisting of formulae L-1 to L-23, wherein:
X³ and X⁴ are independently selected from the group consisting of CH and N;
R¹² and R¹³ are independently selected from the group consisting of hydrogen and C₁-C₆-alkyl; or
R¹² and R¹³, taken together with the carbon atom to which they are attached, form a C₃-C₁₀-cycloalkyl ring;
R¹⁴ is hydrogen or C₁-C₆-alkyl;
R¹⁵ is hydrogen;
R¹⁶ is C₁-C₆-alkyl;
R¹⁷ is hydrogen;
f is an integer selected from 1, 2, 5, 6, 7, 8, 9
g is an integer selected from 3, 6, 8, 9, 10, 11, 14
h is 2,
i is an integer selected from 0, 1, 2, 3,
k is 3;
m is 1;
n is an integer selected from 8 and 12;
p is an integer selected from 0, 1, and 8;
q is 7;
r is an integer selected from 0 and 1;
s is 4;
t is 9;
u is 4;
v is 1;
w is 4;
x is an integer selected from 2 and 4;
y is an integer selected from 1 and 3;
z is 1;
aa is an integer selected from 0, 1, and 8;
Y¹ is —O— or —NH—;
Y² is —O—, —NH—, —C₁-C₆-alkyldiyl- or —NH—C₁-C₆-alkyldiyl-;
Y³ is absent, —O—C₁-C₆-alkyldiyl- or carbonyl;
Y⁴ is —O—, —NH—, —N(C₁-C₆-alkyl)- or —C₁-C₆-alkyldiyl-;
Y⁵ is absent or carbonyl;
Y⁶ is absent, carbonyl, —O—, —NHC(O)—, —C(O)—N(C₁-C₆-alkyl)- or —C(O)NH—;
Y⁷ is absent or —C₁-C₆-alkyldiyl-;
Y⁸ is absent or —O—;
Y⁹ is —NH—; and
each occurrence of a wavy line indicates the point of attachment of the linker to the targeting ligand or to the degron; and
wherein said degron is selected from the group consisting of formulae (DG-1) and (DG-2), wherein:
X⁵ is CH or N;
X⁶ is CH₂ or C(O);
R¹⁸ is hydrogen;
Y¹⁰ is a covalent bond, —O— or —NH—; and
the wavy line indicates the point of attachment to the linker.

In a preferred embodiment, the present invention provides a compound of formula (I) as described herein, or a pharmaceutically acceptable salt thereof, wherein said targeting ligand is of formula (TL), wherein:
R¹ is selected from the group consisting of hydrogen and halogen;
R² is hydrogen;
R³ is hydroxy;
Z¹ is absent;
Cy¹ is 3-14 membered heterocyclyl optionally substituted with R⁴;
Z² is:
   (i) absent;
   (ii) carbonyl; or
   (iii) —C(O)NHCH₂—;
Cy² is:
   (i) C₆-C₁₀-aryl;
   (ii) 3-14 membered heterocyclyl; or
   (iii) 5-14 membered heteroaryl;
Z³ is —X²(CH₂)ₑ—;
Cy³ is 3-14 membered heterocyclyl;
e is an integer selected from 0, 1 and 2;
X² is:
   (i) absent; or
   (ii) —O—;
R⁴ is C₆-C₁₀-aryl; and
the wavy line indicates the point of attachment to the linker;
wherein said linker is a covalent bond or is selected from the group consisting of formulae L-4, L-8, L-13, and L-23, wherein:
i is an integer selected from 0, 2, and 3;
p is an integer selected from 0 and 1;
aa is 0;
Y⁵ is absent;
Y⁶ is absent, carbonyl, —O— or —C(O)—N(C₁-C₆-alkyl)-;
Y⁸ is absent or —O—; and
each occurrence of a wavy line indicates the point of attachment of the linker to the targeting ligand or to the degron; and
wherein said degron is selected from the group consisting of formulae (DG-1) and (DG-2), wherein:
X⁵ is CH;
X⁶ is C(O);
R¹⁸ is hydrogen;
Y¹⁰ is —NH—; and
the wavy line indicates the point of attachment to the linker.

In a particularly preferred embodiment, the present invention provides a compound of formula (I) as described herein, or a pharmaceutically acceptable salt thereof, wherein said targeting ligand is of formula (TL), wherein:
R¹ is selected from the group consisting of hydrogen and fluoro;
R² is hydrogen;
R³ is hydroxy;
Z¹ is absent;
Cy¹ is selected from the group consisting of:

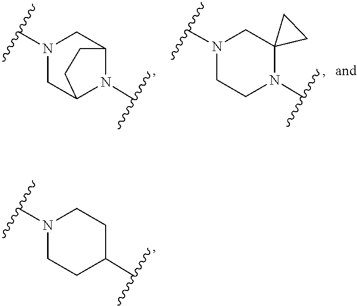 [SMILES: *N1CC2CCC(C1)N2* ; *N1CCN(*)C2(CC2)C1 ; *C1CCN(*)CC1]

optionally substituted with R⁴; wherein each wavy line indicates the point of attachment to Z² or to the remainder of formula (TL);
Z² is:
   (i) absent;
   (ii) carbonyl; or
   (iii) —C(O)NHCH₂—;
Cy² is
   (i) phenyl;
   (ii) 3-14 membered heterocyclyl selected from:

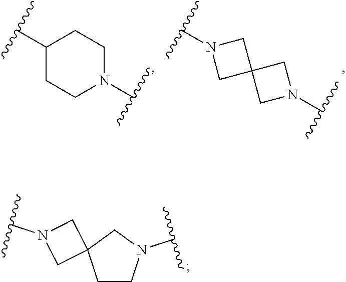 [SMILES: *C1CCN(*)CC1 ; *N1CC2(C1)CN(*)C2 ; *N1CCC2(C1)CN(*)C2]

wherein each wavy line indicates the point of attachment to Z² or Z³; or
(iii) pyrimidinyl;
Z³ is —X²(CH₂)ₑ—;
Cy³ is 3-14 membered heterocyclyl selected from:

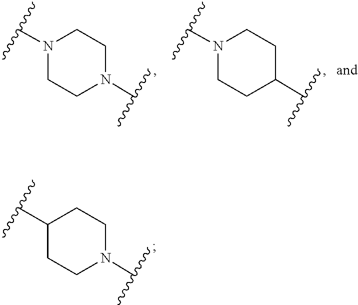 [SMILES: *N1CCN(*)CC1 ; *C1CCN(*)CC1 ; *C1CCN(*)CC1]

wherein each wavy line indicates the point of attachment to Z³ or the linker;
e is an integer selected from 0, 1 and 2;
X² is:
   (i) absent; or
   (ii) —O—;
R⁴ is phenyl; and
the wavy line indicates the point of attachment to the linker;
wherein said linker is a covalent bond or is selected from the group consisting of formulae L-4, L-8, L-13, and L-23, wherein:
i is an integer selected from 0, 2, and 3;
p is an integer selected from 0 and 1;
aa is 0;
Y⁵ is absent;
Y⁶ is absent, carbonyl, —O— or —C(O)—NCH₃—;
Y⁸ is absent or —O—; and
each occurrence of a wavy line indicates the point of attachment of the linker to the targeting ligand or to the degron; and
wherein said degron is selected from the group consisting of formulae (DG-1) and (DG-2), wherein:
X⁵ is CH;
X⁶ is C(O);
R¹⁸ is hydrogen;
Y¹⁰ is —NH—; and
the wavy line indicates the point of attachment to the linker.

In one embodiment, the present invention provides a compound of formula (I) as described herein, or a pharmaceutically acceptable salt thereof, wherein R¹ and R² are each independently selected from the group consisting of hydrogen and halogen.

In a preferred embodiment, the present invention provides a compound of formula (I) as described herein, or a pharmaceutically acceptable salt thereof, wherein:
R¹ is selected from the group consisting of hydrogen and halogen; and
R² is hydrogen.

In a particularly preferred embodiment, the present invention provides a compound of formula (I) as described herein, or a pharmaceutically acceptable salt thereof, wherein:
R¹ is selected from the group consisting of hydrogen and fluoro; and
R² is hydrogen.

In one embodiment, the present invention provides a compound of formula (I) as described herein, or a pharmaceutically acceptable salt thereof, wherein R³ is selected from the group consisting of amino and hydroxy.

In a preferred embodiment, the present invention provides a compound of formula (I) as described herein, or a pharmaceutically acceptable salt thereof, wherein R³ is hydroxy.

In one embodiment, the present invention provides a compound of formula (I) as described herein, or a pharmaceutically acceptable salt thereof, wherein Z¹ is:
(i) absent;
(ii) —O—; or
(iii) —O—C₁-C₆-alkyldiyl-CH(C₆-C₁₀-aryl)-C₁-C₆-alkyldiyl-NHC(O)—.

In a preferred embodiment, the present invention provides a compound of formula (I) as described herein, or a pharmaceutically acceptable salt thereof, wherein Z¹ is absent.

In one embodiment, the present invention provides a compound of formula (I) as described herein, or a pharmaceutically acceptable salt thereof, wherein Cy¹ is 3-14 membered heterocyclyl optionally substituted with R⁴; and wherein R⁴ is C₆-C₁₀-aryl.

In a preferred embodiment, the present invention provides a compound of formula (I) as described herein, or a pharmaceutically acceptable salt thereof, wherein Cy¹ is selected from the group consisting of:

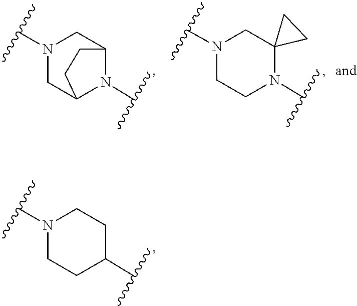 [SMILES: *N1CC2CCC(C1)N2* ; *N1CCN(*)C2(CC2)C1 ; *C1CCN(*)CC1]

optionally substituted with R⁴; wherein each wavy line indicates the point of attachment to Z² or to the remainder of formula (TL); and wherein R⁴ is phenyl.

In one embodiment, the present invention provides a compound of formula (I) as described herein, or a pharmaceutically acceptable salt thereof, wherein Z² is:
(i) absent;
(ii) carbonyl;
(iii) —O(CH₂)ₐ;
(iv) —C(O)NH(CH₂)_{b}—;
(v) —(CH₂)_{c}NHC(O)(CH₂)_{d}X¹—;
(vi) —O—C₁-C₆-alkyldiyl-C(O)NH—; or
(vii) —CH₂N(C₁-C₆-alkyl)CH₂—; wherein

a is 0, 1 or 2;

b is 0 or 1;

c and d are each independently an integer selected from 0, 1, 2 and 3; and

X¹ is:
(i) absent;
(ii) —NH—; or
(iii) —O—.

In a preferred embodiment, the present invention provides a compound of formula (I) as described herein, or a pharmaceutically acceptable salt thereof, wherein Z² is:
(i) absent;
(ii) carbonyl; or
(iii) —C(O)NHCH₂—.

In one embodiment, the present invention provides a compound of formula (I) as described herein, or a pharmaceutically acceptable salt thereof, wherein Cy² is:
(i) absent;
(ii) C₆-C₁₀-aryl optionally substituted with R⁵;
(iii) C₃-C₁₀-cycloalkyl;
(iv) 3-14 membered heterocyclyl; or
(v) 5-14 membered heteroaryl; wherein

R⁵ is selected from the group consisting of halogen, C₁-C₆-alkyl, and halo-C₁-C₆-alkyl.

In a preferred embodiment, the present invention provides a compound of formula (I) as described herein, or a pharmaceutically acceptable salt thereof, wherein Cy² is:
(i) C₆-C₁₀-aryl;
(ii) 3-14 membered heterocyclyl; or
(iii) 5-14 membered heteroaryl.

In a particularly preferred embodiment, the present invention provides a compound of formula (I) as described herein, or a pharmaceutically acceptable salt thereof, wherein Cy² is:
(i) phenyl;
(ii) 3-14 membered heterocyclyl selected from:

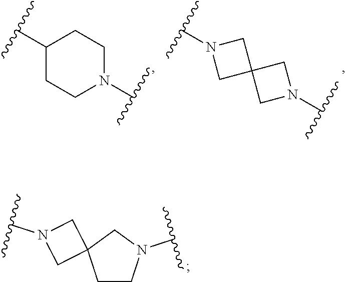 [SMILES: *C1CCN(*)CC1 ; *N1CC2(C1)CN(*)C2 ; *N1CCC2(C1)CN(*)C2]

wherein each wavy line indicates the point of attachment to Z² or Z³; or; or
(iii) pyrimidinyl.

In one embodiment, the present invention provides a compound of formula (I) as described herein, or a pharmaceutically acceptable salt thereof, wherein Z³ is:
(i) absent;
(ii) X²(CH₂)ₑ—; or
(iii) —(CH₂)ₑX²—; wherein

X² is:
(i) absent;
(ii) carbonyl;
(iii) —O—; or
(iv) —NHC(O)—; and

each e is independently an integer selected from 0, 1, 2 and 3.

In a preferred embodiment, the present invention provides a compound of formula (I) as described herein, or a pharmaceutically acceptable salt thereof, wherein Z³ is —X²(CH₂)ₑ—; wherein:

X² is:
(i) absent; or
(ii) —O—; and

e is an integer selected from 0, 1 and 2.

In one embodiment, the present invention provides a compound of formula (I) as described herein, or a pharmaceutically acceptable salt thereof, wherein Cy³ is:
(i) absent;
(ii) C₆-C₁₀-aryl;
(iii) C₃-C₁₀-cycloalkyl; or
(iv) 3-14 membered heterocyclyl.

In a preferred embodiment, the present invention provides a compound of formula (I) as described herein, or a pharmaceutically acceptable salt thereof, wherein Cy³ is 3-14 membered heterocyclyl selected from:

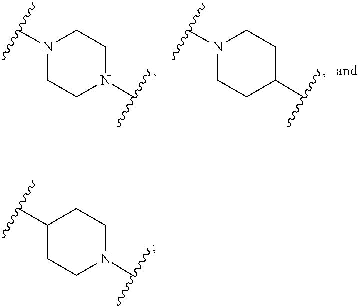 [SMILES: *N1CCN(*)CC1 ; *C1CCN(*)CC1 ; *C1CCN(*)CC1]

wherein each wavy line indicates the point of attachment to Z³ or the linker.

In one embodiment, the present invention provides a compound of formula (I) as described herein, or a pharmaceutically acceptable salt thereof, wherein said compound of formula (I) is selected from Examples 1 to 204.

In one embodiment, the present invention provides a compound of formula (I) as described herein, or a pharmaceutically acceptable salt thereof, wherein said compound of formula (I) is selected from Examples 34, 35, 36, 46, 55, 84, 95, 96, 100, 113, 113, 114, 118, 127, 142, 143, 149, 149, 158, 159, 161, 170, 190, and 191.

In one embodiment, the present invention provides pharmaceutically acceptable salts or esters of the compounds of formula (I) as described herein. In a particular embodiment, the present invention provides pharmaceutically acceptable salts of the compounds according to formula (I) as described herein. In a further particular embodiment, the present invention provides pharmaceutically acceptable esters of the compounds according to formula (I) as described herein. In yet a further particular embodiment, the present invention provides compounds according to formula (I) as described herein.

Furthermore, the invention includes all optical isomers, i.e. diastereoisomers, diastereomeric mixtures, racemic mixtures, all their corresponding enantiomers and/or tautomers as well as their solvates of the compounds of formula I.

The compounds of formula I may contain one or more asymmetric centers and can therefore occur as racemates, racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within this invention. The present invention is meant to encompass all such isomeric forms of these compounds. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

In the embodiments, where optically pure enantiomers are provided, optically pure enantiomer means that the compound contains >90% of the desired isomer by weight, particularly >95% of the desired isomer by weight, or more particularly >99% of the desired isomer by weight, said weight percent based upon the total weight of the isomer(s) of the compound. Chirally pure or chirally enriched compounds may be prepared by chirally selective synthesis or by separation of enantiomers. The separation of enantiomers may be carried out on the final product or alternatively on a suitable intermediate.

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

Processes of Manufacturing

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)—insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, N.Y. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

Bifunctional protein degrader molecules of formula (I), or their pharmaceutical acceptable salts, polymorphic forms, prodrugs, solvate forms and isotope containing derivatives thereof, may be prepared by the general approaches described below (Scheme 1, Scheme 2 and Scheme 3), together with synthetic methods known in the art, or modifications and derivatizations that are familiar to those of ordinary skill in the art.

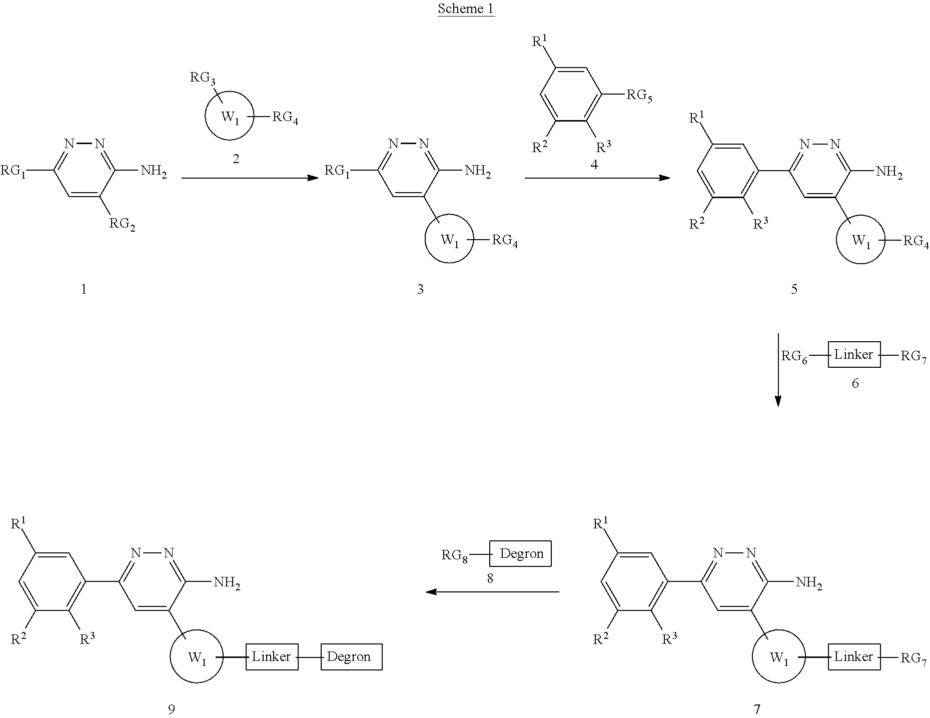

The sequence of the reactions for the preparation of bifunctional protein degrader molecule may be modified as shown below (Scheme 2 and Scheme 3).

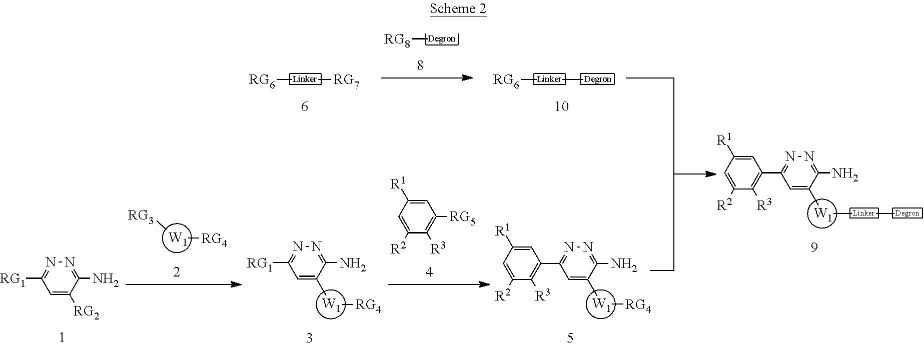

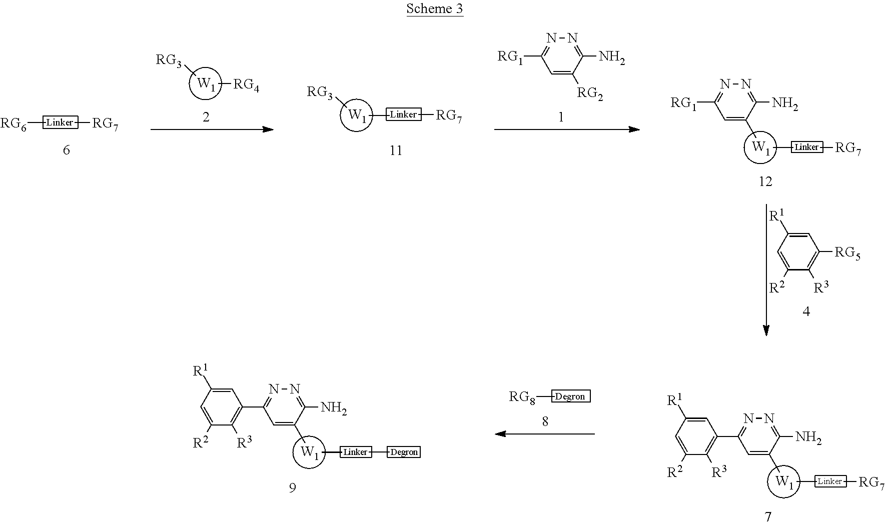

Degron is a moiety of formula (DG-1), (DG-2), (DG-3) or (DG-4) as described herein.

In Schemes 1, 2 and 3, starting material 1 is commercially available. For example, starting material 1 is 4-bromo-6-chloropyridazin-3-amine.

In Schemes 1, 2 and 3, reactant 2 is commercially available or can be prepared as described in the prior art (see e.g., WO2016138114) or in analogy to the procedure described in the Examples.

W₁ is a moiety of formula

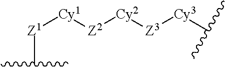

as described herein.

In Schemes 1, 2 and 3, reactant 4 is commercially available. For example, reactant 4 is an appropriately-substituted ortho-phenol boronic acid.

In Scheme 1, 2 and 3, reactant 6 is commercially available or can be prepared in analogy to literature procedures or the procedures described in the Examples.

In general, compounds 5 in Scheme 1 and 2 or compound 7 in Scheme 3 can be prepared from 4-bromo-6-chloropyridazin-3-amine. Substituents can be introduced at C-4 via palladium-catalyzed cross coupling or nucleophilic aromatic substitution, followed by palladium-catalyzed cross coupling of appropriately-substituted ortho phenol boronic acids or boronic esters at C-6.

SNAr with Amines

In certain examples, for the chemistry described in Scheme 1, 2 and 3, RG₂ is a halogen, preferentially a bromide, and RG₃ is a suitable nucleophile such as —NH₂ or —NH—. In a typical procedure, a RG₂ containing intermediate is reacted with a RG₃ containing intermediate in a suitable solvent. Suitable solvents include, but are not limited to, water, ethers such as THF, glyme, and the like; chlorinated solvents such as DCM, 1,2-dichloroethane (DCE) or CHCl₃ and the like; toluene, benzene and the like; DMF, NMP, DMSO, MeCN. If desired, mixtures of these solvents are used. To facilitate the reaction a base may be added. Suitable bases include, but are not limited to, Cs₂CO₃, K₂CO₃ and the like; TEA, DIPEA and the like. The above process may be carried out at temperatures between about 20° C. and about 200° C. Preferentially, the reaction is carried out between about 50° C. and about 130° C.

Buchwald Coupling

In certain examples, for the chemistry described in Scheme 1, 2 and 3, RG₂ is a halogen such as chlorine or bromine, preferably bromine, and RG₃ is a nucleophile such as —NH₂, —NH— or —OH. In a typical procedure, a RG₂-containing intermediate is reacted with a RG₅-containing intermediate in a suitable solvent in the presence of a suitable catalyst and a base. Suitable solvents include, but are not limited to, water, ethers such as THF, glyme, dioxane and the like; chlorinated solvents such as DCM, 1,2-dichloroethane (DCE) or CHCl₃ and the like; toluene, benzene and the like; alcohols such as methanol, ethanol, isopropanol, tert-butanol and the like; DMF, NMP, DMSO, MeCN. If desired, mixtures of these solvents are used. Preferentially, dioxane or isopropanol are used. Suitable catalyst includes, but is not limited to tetrakis(triphenylphosphine)Pd, RuPhosPd G3, bis(diphenylphosphino)ferrocene] dichloro Pd(II), BrettPhosPd G3. Suitable bases include, but are not limited to, Na₂CO₃, K₂CO₃, Cs₂CO₃, K₂PO₄, Na₂PO₄. The above process may be carried out at temperatures between 20° C. and about 150° C. Preferably, the reaction is carried out between 60° C. and 120° C.

Examples of commercially available building blocks containing —NH₂ or —NH— as RG₃ are depicted in Scheme 4 but are not limited to these examples:

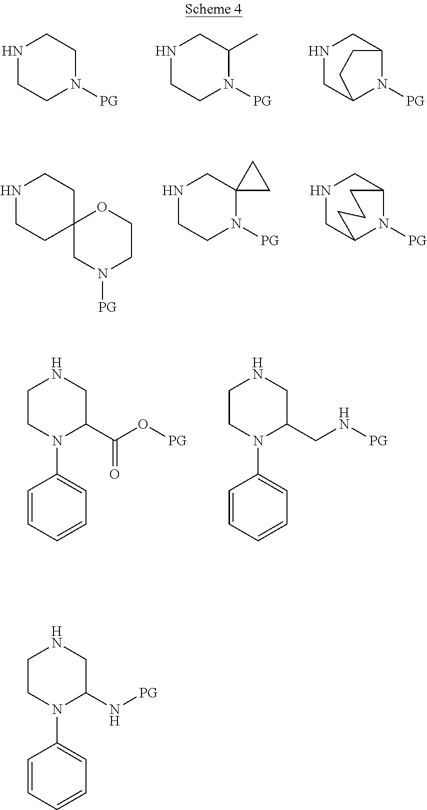

Non commercially available building blocks containing —NH₂ or —NH— as RG₃ can be obtained for example applying the synthetic routes outlined in Schemes 5a-k, wherein PG is a suitable protecting group.

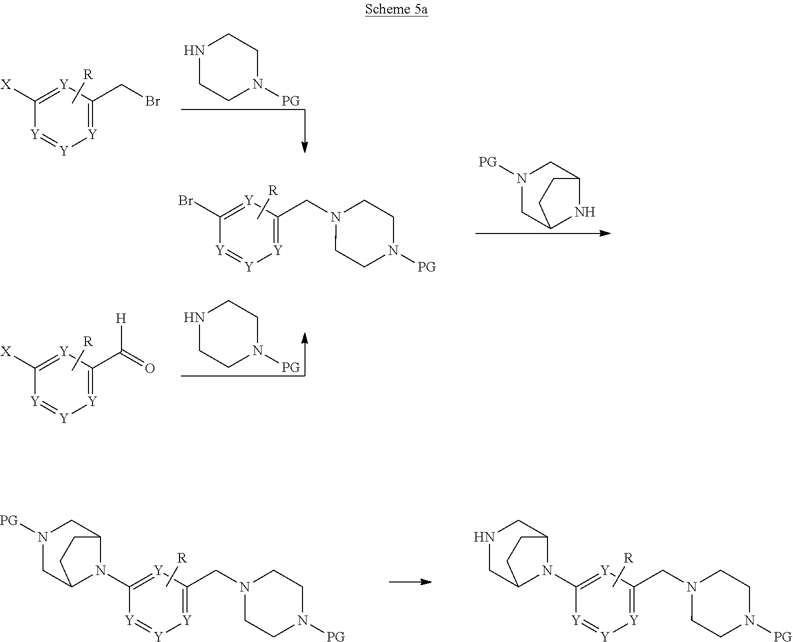

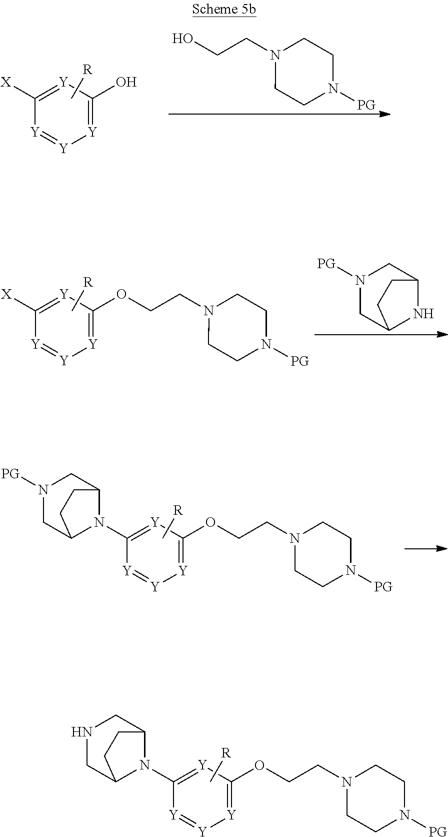

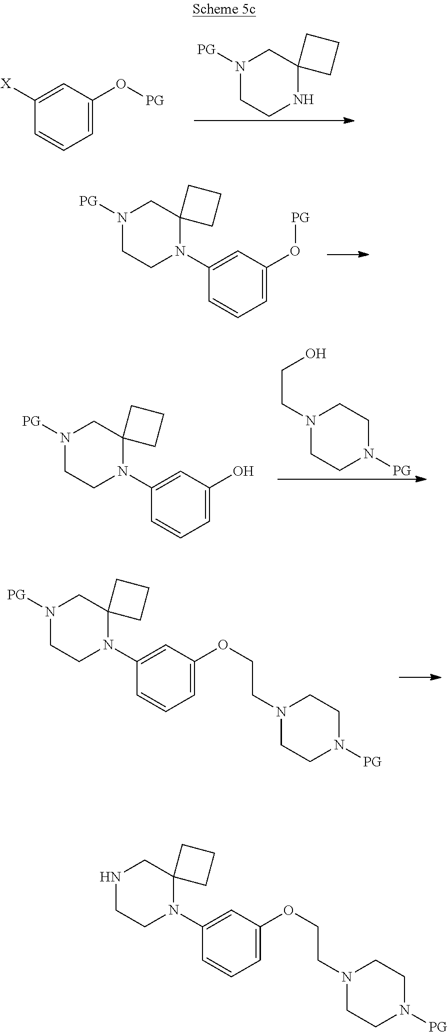

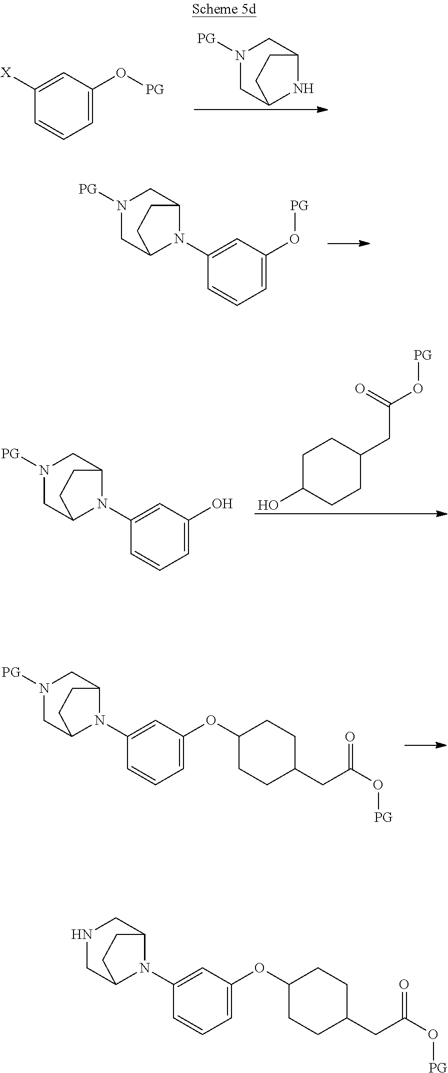

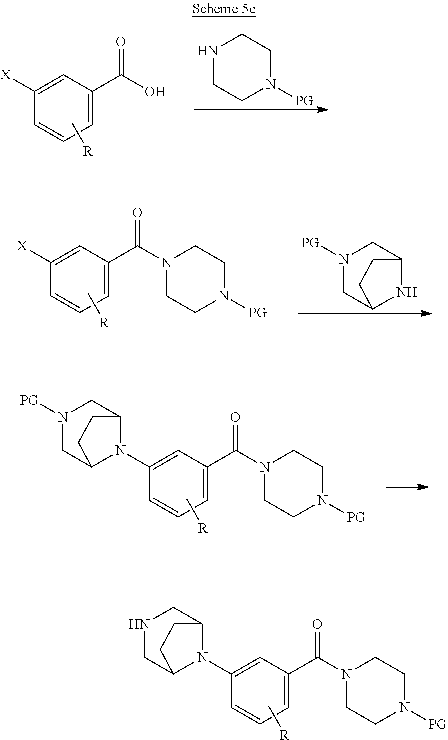

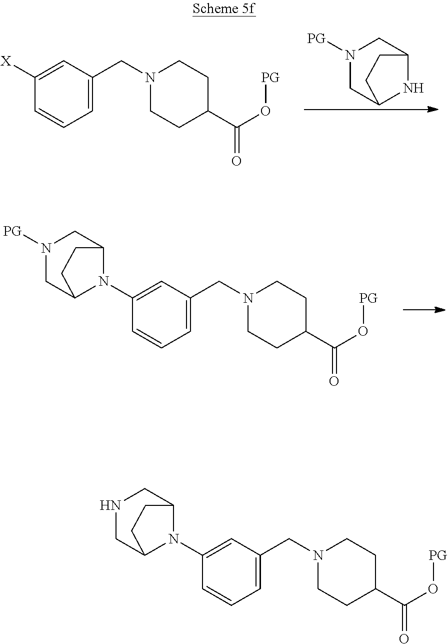

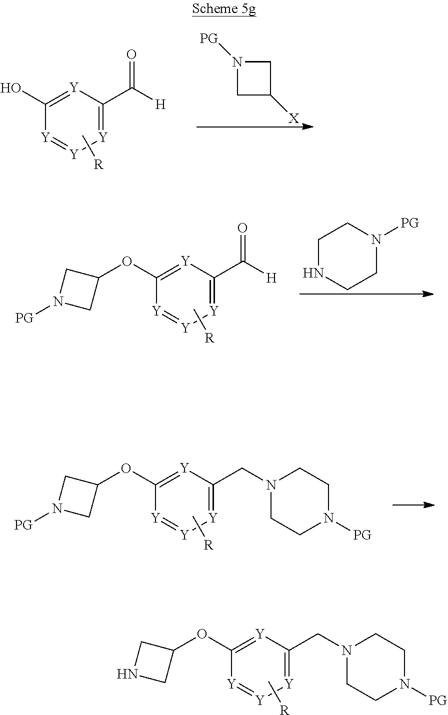

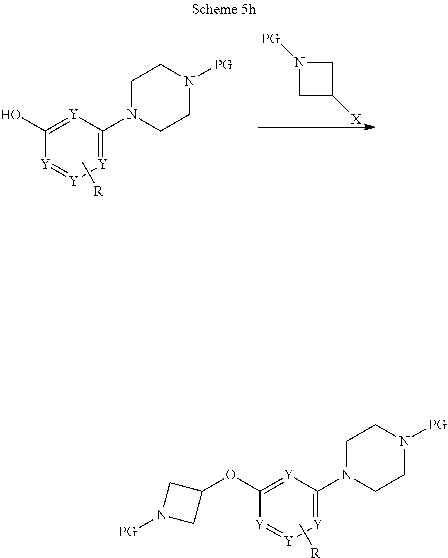

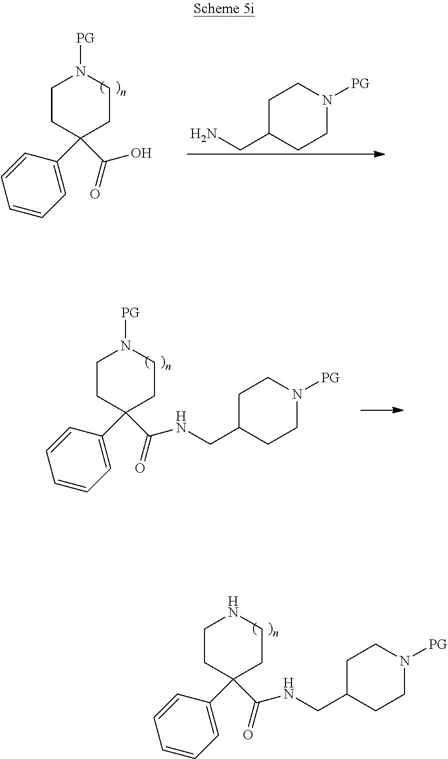

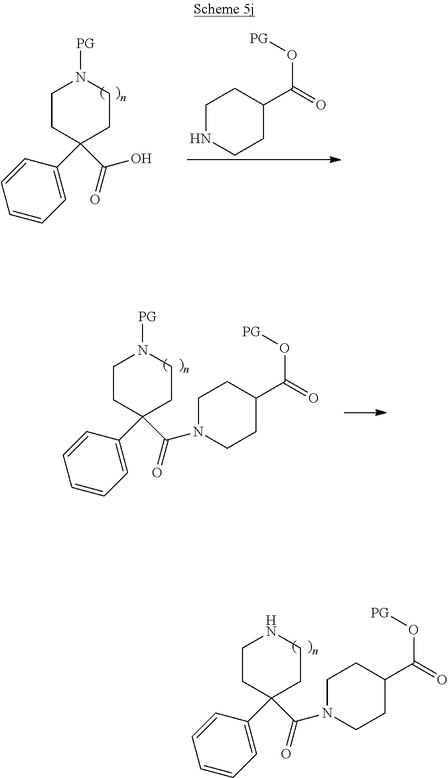

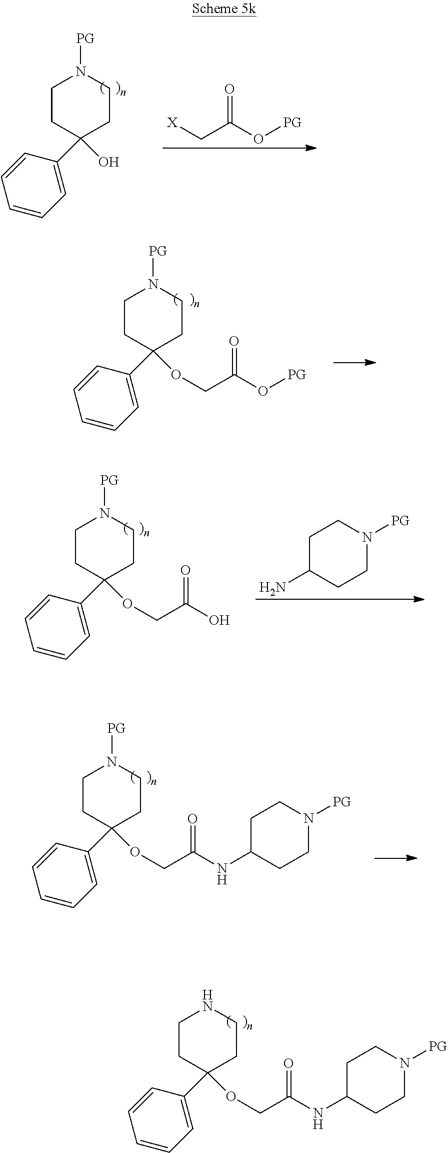

SNAr with OH

In certain examples, for the chemistry described in Scheme 1, 2 and 3, RG₂ is a halogen, preferentially a bromide, and RG₃ is a hydroxy group —OH. In a typical procedure, a RG₂ containing intermediate is reacted with a RG₃ containing intermediate in a suitable solvent. Suitable solvents include, but are not limited to, ethers such as THF, glyme, and the like; chlorinated solvents such as DCM, 1,2-dichloroethane (DCE) or CHCl₃ and the like; toluene, benzene and the like; DMF, NMP, DMSO MeCN. If desired, mixtures of these solvents are used. To facilitate the reaction a base may be added. Suitable bases include, but are not limited to, NaH, Cs₂CO₃, K₂CO₃ and the like; TEA, DIPEA and the like. Preferably, the base added is NaH. The above process may be carried out at temperatures between about 20° C. and about 200° C. Preferably, the reaction is carried out between about 50° C. and about 130° C.

Non commercially available building blocks containing —OH as RG₃ can be obtained using standard chemistry as depicted in Scheme 6. PG is a suitable protecting group.

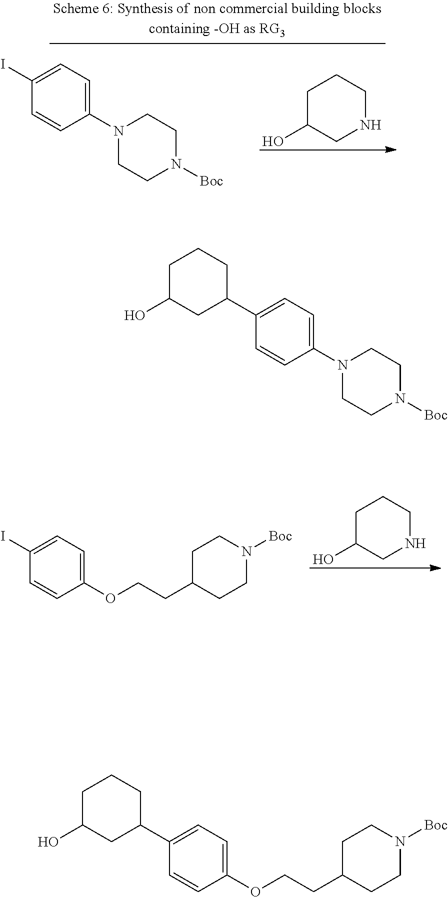 [SMILES: CC(C)(C)OC(=O)N1CCN(c2ccc(I)cc2)CC1 ; OC1CCCNC1 ; CC(C)(C)OC(=O)N1CCN(c2ccc(C3CCCC(O)C3)cc2)CC1 ; CC(C)(C)OC(=O)N1CCC(CCOc2ccc(I)cc2)CC1 ; OC1CCCNC1 ; CC(C)(C)OC(=O)N1CCC(CCOc2ccc(C3CCCC(O)C3)cc2)CC1]

Suzuki Coupling

In certain examples, for the chemistry described in Scheme 1, 2 and 3, RG₁ is a halogen such as chlorine or bromine, preferably chlorine, and RG₅ is a boron-containing moiety, preferably a boronic acid or a boronic ester. In a typical procedure, a RG₁-containing intermediate is reacted with a RG₅-containing intermediate in a suitable solvent in the presence of a suitable catalyst and a base. Suitable solvents include, but are not limited to, water, ethers such as THF, glyme, dioxane and the like; chlorinated solvents such as DCM, 1,2-dichloroethane (DCE) or CHCl₃ and the like; toluene, benzene and the like; alcohols such as methanol, ethanol, isopropanol, tert-butanol and the like; DMF, NMP, DMSO, MeCN. If desired, mixtures of these solvents are used. Preferentially, dioxane or isopropanol are used. Suitable catalyst includes, but is not limited to tetrakis(triphenylphosphine)Pd, RuPhosPd G3, bis(diphenylphosphino)ferrocene] dichloro Pd(II), BrettPhosPd G3. Suitable bases include, but are not limited to, Na₂CO₃, K₂CO₃, Cs₂CO₃, K₂PO₄, Na₂PO₄. The above process may be carried out at temperatures between 20° C. and about 150° C. Preferably, the reaction is carried out between 60° C. and 120° C.

In general, compounds 7 and 8 in Scheme 1 or compounds 10 and 9 in Scheme 2 or compounds 11 and 9 in Scheme 3 can be prepared via amide coupling reaction, reductive amination, alkylation reaction, urea formation.

Amide Formation

In certain examples, for the chemistry described in Scheme 1, 2 and 3, RG₄ is a moiety containing a —COOH group and RG₆ is a moiety containing a suitable amine group. In a typical procedure, a RG₄-containing intermediate is reacted with a RG₆-containing intermediate in a suitable solvent in the presence of a suitable amide coupling reagent. Suitable solvents include, but are not limited to, water, ethers such as THF, glyme, and the like; chlorinated solvents such as DCM, 1,2-dichloroethane (DCE) or CHCl₃ and the like; toluene, benzene and the like; DMF, NMP, DMSO MeCN. If desired, mixtures of these solvents are used. Preferentially, DMF or DCM is used. A suitable amide coupling reagent include, but are not limited to, DCC, EDC, HATU, HBTU, PyBOP and the like. A base is often added to the reaction. Suitable bases include, but are not limited to, TEA, DIPEA, and the like. The above process may be carried out at temperatures between −78° C. and about 150° C. Preferably, the reaction is carried out between 0° C. and 50° C.

Alternatively, RG₄ is a moiety containing a —NH₂ or —NH— group RG₆ is a moiety containing a —COOH group.

The same considerations apply for the coupling of the moieties RG₇ and RG₈.

Alkylation

In certain examples, for the chemistry described in Scheme 1, 2 and 3, RG₄ is a moiety containing a —NH₂ or —NH— group and RG₆ is a moiety containing leaving group such as a halogen or a mesylate. In a typical procedure, a RG₄-containing intermediate is reacted with a RG₆-containing intermediate in a suitable solvent. Suitable solvents include, but are not limited to, water, ethers such as THF, glyme, and the like; chlorinated solvents such as DCM, 1,2-dichloroethane (DCE) or CHCl₃ and the like; toluene, benzene and the like; DMF, NMP, DMSO MeCN. If desired, mixtures of these solvents are used. Preferentially, DMSO or DMF is used. A base might be added to the reaction. Suitable bases include, but are not limited to, Na₂CO₃, K₂CO₃, and the like, or TEA, DIPEA, and the like. The above process may be carried out at temperatures between −10° C. and about 150° C. Preferably, the reaction is carried out between 0° C. and 50° C.

Alternatively, RG₄ is a moiety containing a leaving group such a a halogen or mesylate and RG₆ is a moiety containing a —NH₂ or —NH— group.

The same considerations apply for the coupling of the moieties RG₇ and RG₈.

Reductive Amination 1

In certain examples, for the chemistry described in Scheme 1, 2 and 3, RG₄ is a moiety containing a —CHO or a —CO— group and RG₆ is a moiety containing a suitable amine group. In a typical procedure, a RG₄-containing intermediate is reacted with a RG₆-containing intermediate in a suitable solvent in the presence of a suitable reducing reagent. Suitable solvents include, but are not limited to, water, ethers such as THF, DME, glyme, and the like; chlorinated solvents such as DCM, 1,2-dichloroethane (DCE) or CHCl₃ and the like; toluene, benzene and the like; alcohols such as methanol, ethanol, isopropanol, tert-butanol and the like; toluene, benzene and the like. If desired, mixtures of these solvents are used. Preferentially, DMF or DCM is used. A suitable reducing reagent include, but are not limited to, sodium borohydride, sodium triacetoxyborohydride, sodium cyanoborohydride and the like; mixtures of dibutyltindichloride and trimethyl(phenyl)silane and the like. An acid is often added to the reaction. Suitable acids include, but are not limited to, acetic acid or formic acid, and the like. The above process may be carried out at temperatures between −78° C. and about 150° C. Preferably, the reaction is carried out between 0° C. and 50° C.

Alternatively, RG₄ is a moiety containing a a —NH₂ or —NH— group and RG₆ is a moiety containing a —CHO or a —CO— group.

The same considerations apply for the coupling of the moieties RG₇ and RG₈.

Urea Formation

In certain examples, for the chemistry described in Scheme 1, 2 and 3, RG₄ is a moiety containing an activated carbamate or a carboxylic azide group or an isocyanate and RG₆ is a moiety containing a suitable amine group. Activated carbamate groups include, but are not limited to, (4-nitrophenyl)carbamate, (pentafluorophenyl)carbamate. In a typical procedure, a RG₄-containing intermediate is reacted with a RG₆-containing intermediate in a suitable solvent. Suitable solvents include, but are not limited to, water, ethers such as THF, glyme, and the like; chlorinated solvents such as DCM, 1,2-dichloroethane (DCE) or CHCl₃ and the like; toluene, benzene and the like; DMF, NMP, DMSO MeCN. If desired, mixtures of these solvents are used. Preferentially, DMF or DCM is used. A suitable reducing reagent include, but are not limited to, sodium borohydride, sodium triacetoxyborohydride, sodium cyanoborohydride and the like; mixtures of dibutyltindichloride and trimethyl(phenyl)silane and the like. An acid is often added to the reaction. Suitable acids include, but are not limited to, acetic acid or formic acid, and the like. The above process may be carried out at temperatures between −78° C. and about 150° C. Preferably, the reaction is carried out between 0° C. and 50° C.

Alternatively, RG₄ is a moiety containing a —NH₂ or —NH— group and RG₆ is a moiety containing an activated carbamate or a carboxylic azide group or an isocyanate.

The same considerations apply for the coupling of the moieties RG₇ and RG₈.

Isolation and Purification of the Compounds

Isolation and purification of the compounds and intermediates described herein can be effected, if desired, by any suitable separation or purification procedure such as, for example, filtration, extraction, crystallization, column chromatography, thin-layer chromatography, thick-layer chromatography, preparative low or high-pressure liquid chromatography or a combination of these procedures. Specific illustrations of suitable separation and isolation procedures can be had by reference to the preparations and examples herein below. However, other equivalent separation or isolation procedures could, of course, also be used. Racemic mixtures of chiral compounds of formula I can be separated using chiral HPLC. Racemic mixtures of chiral synthetic intermediates may also be separated using chiral HPLC.

In cases where the compounds of formula I are basic they may be converted to a corresponding acid addition salt. The conversion is accomplished by treatment with at least a stoichiometric amount of an appropriate acid, such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Typically, the free base is dissolved in an inert organic solvent such as diethyl ether, ethyl acetate, chloroform, ethanol or methanol and the like, and the acid added in a similar solvent. The temperature is maintained between 0° C. and 50° C. The resulting salt precipitates spontaneously or may be brought out of solution with a less polar solvent.

It will be appreciated that the compounds of general formula I in this invention may be derivatised at functional groups to provide derivatives which are capable of conversion back to the parent compound in vivo.

Using the Compounds of the Invention

The compounds of Formula I can be used in an effective amount to treat a host, including a human, affected by SMARCA2-mediated disorders. More particularly, the compounds of Formula I can be used in an effective amount to treat a subject, in particular a human, affected by cancer.

In one aspect, the present invention provides a compound of formula (I) described herein, or a pharmaceutically acceptable salt thereof, for use as therapeutically active substance.

In a further aspect, the present invention provides a compound of formula (I) described herein, or a pharmaceutically acceptable salt thereof, for use in the treatment of SMARCA2-mediated disorders.

In a further aspect, the present invention provides a method of treating SMARCA2-mediated disorders in a subject, comprising administering a compound of formula (I) as described herein, or a pharmaceutically acceptable salt thereof, to the subject.

In a further aspect, the present invention provides the use of a compound of formula (I) described herein, or a pharmaceutically acceptable salt thereof, in a method of treating SMARCA2-mediated disorders in a subject.

In a further aspect, the present invention provides the use of a compound of formula (I) described herein, or a pharmaceutically acceptable salt thereof, for the manufacture of a medicament for treating SMARCA2-mediated disorders in a subject.

The term “SMARCA2-mediated disorder” is characterized by the participation of the SMARCA2 protein in the inception, manifestation of one or more symptoms or disease markers, severity, or progression of a disorder.

SMARCA2-mediated disorders include cancers, including, but not limited to acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, liver cancer, lung cancer, lymphagioendotheliosarcoma, lymphangiosarcoma, lymphoblastic leukemia, lymphoma (Hodgkin's and non-Hodgkin's; Burkitt's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

Co-Administration of Compounds of Formula (I) and Other Agents

The compounds of formula (I) or salts thereof or a compound disclosed herein or a pharmaceutically acceptable salt thereof may be employed alone or in combination with other agents for treatment. For example, the second agent of the pharmaceutical combination formulation or dosing regimen may have complementary activities to the compound of formula (I) such that they do not adversely affect each other. The compounds may be administered together in a unitary pharmaceutical composition or separately. In one embodiment a compound or a pharmaceutically acceptable salt can be co-administered with a cytotoxic agent to treat proliferative diseases and cancer.

The term “co-administering” refers to either simultaneous administration, or any manner of separate sequential administration, of a compound of formula (I) or a salt thereof or a compound disclosed herein or a pharmaceutically acceptable salt thereof and a further active pharmaceutical ingredient or ingredients, including cytotoxic agents and radiation treatment. If the administration is not simultaneous, the compounds are administered in a close time proximity to each other. Furthermore, it does not matter if the compounds are administered in the same dosage form, e.g. one compound may be administered topically and another compound may be administered orally.

Typically, any agent that has activity against a SMARCA2-mediated disease or condition being treated may be co-administered. Examples of such agents can be found in Cancer Principles and Practice of Oncology by V. T. Devita and S. Heilman (editors), 6th edition (Feb. 15, 2001), Lippincott Williams & Wilkins Publishers. A person of ordinary skill in the art would be able to discern which combinations of agents would be useful based on the particular characteristics of the drugs and the disease involved.

In one aspect, the present invention provides a pharmaceutical composition described herein, further comprising an additional therapeutic agent.

In one embodiment, said additional therapeutic agent is a chemotherapeutic agent.

In one embodiment, said additional therapeutic agent is a cytotoxic agent.

The term “cytotoxic agent” as used herein refers to a substance that inhibits or prevents a cellular function and/or causes cell death or destruction. Cytotoxic agents include, but are not limited to, radioactive isotopes (At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; Eiziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin I and cryptophycin 8); adrenocorticosteroids (including prednisone and prednisolone); cyproterone acetate; 5a-reductases including finasteride and dutasteride); vorinostat, romidepsin, panobinostat, valproic acid, mocetinostat dolastatin; aldesleukin, talc duocarmycin (including the synthetic analogs, KW-2189 and CBI-TM I); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin [symbol] T and calicheamicin coll (Angew Chem. Inti. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, 111.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-II; topoisomerase inhibitor RFS 2000; difluoromethylomithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LYl 17018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase I inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idee), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgGi λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al, J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFRantagonists include OSI-774 (CP-358774, erlotinib, TARCEVA® Genentech/OSI Pharmaceuticals); PD 183805 (Cl 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(I-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule FIER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-I inhibitors such as antisense agent ISIS-5132 available from ISIS Pharmaceuticals which inhibit Raf-I signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor Cl-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Wamer-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-ICl I (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (PEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFa) blockers such as etanercept (Enbrel), infliximab (Remicade), adalimumab (Humira), certolizumab pegol (Cimzia), golimumab (Simponi), Interleukin I (IL-I) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-Ml prime; Secreted homotrimeric LTa3 and membrane bound heterotrimer LTa I/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH3, or famesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTQNEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifamib (RI 1577); orafenib, ABT510; Bcl-2 inhibitor such as oblimersen sodium (GENASENSE®); pixantrone; famesyltransferase inhibitors such as lonafamib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Pharmaceutical Compositions and Administration

The compounds of formula I and the pharmaceutically acceptable salts can be used as therapeutically active substances, e.g. in the form of pharmaceutical preparations. The pharmaceutical preparations can be administered orally, e.g. in the form of tablets, coated tablets, dragées, hard and soft gelatin capsules, solutions, emulsions or suspensions. The administration can, however, also be effected rectally, e.g. in the form of suppositories, or parenterally, e.g. in the form of injection solutions.

The compounds of formula I and the pharmaceutically acceptable salts thereof can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragées and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing a compound of formula I or a pharmaceutically acceptable salt thereof and a therapeutically inert carrier are also provided by the present invention, as is a process for their production, which comprises bringing one or more compounds of formula I and/or pharmaceutically acceptable salts thereof and, if desired, one or more other therapeutically valuable substances into a galenical administration form together with one or more therapeutically inert carriers.

The dosage can vary within wide limits and will, of course, have to be adjusted to the individual requirements in each particular case. In the case of oral administration, the dosage for adults can vary from about 0.01 mg to about 1000 mg per day of a compound of general formula I or of the corresponding amount of a pharmaceutically acceptable salt thereof. The daily dosage may be administered as single dose or in divided doses and, in addition, the upper limit can also be exceeded when this is found to be indicated.

The following examples illustrate the present invention without limiting it, but serve merely as representative thereof. The pharmaceutical preparations conveniently contain about 1-500 mg, particularly 1-100 mg, of a compound of formula I. Examples of compositions according to the invention are:

### Example A

Tablets of the following composition are manufactured in the usual manner:

TABLE 2
possible tablet composition

mg/tablet

| ingredient | 5 | 25 | 100 | 500 |
|---|---|---|---|---|
| Compound of formula I | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 831 |

Manufacturing Procedure

1. Mix ingredients 1, 2, 3 and 4 and granulate with purified water.

2. Dry the granules at 50° C.

3. Pass the granules through suitable milling equipment.

4. Add ingredient 5 and mix for three minutes; compress on a suitable press.

### Example B-1

Capsules of the following composition are manufactured:

TABLE 3
possible capsule ingredient composition

mg/capsule

| ingredient | 5 | 25 | 100 | 500 |
|---|---|---|---|---|
| Compound of formula I | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

Manufacturing Procedure

1. Mix ingredients 1, 2 and 3 in a suitable mixer for 30 minutes.

2. Add ingredients 4 and 5 and mix for 3 minutes.

3. Fill into a suitable capsule.

The compound of formula I, lactose and corn starch are firstly mixed in a mixer and then in a comminuting machine. The mixture is returned to the mixer; the talc is added thereto and mixed thoroughly. The mixture is filled by machine into suitable capsules, e.g. hard gelatin capsules.

### Example B-2

Soft Gelatin Capsules of the following composition are manufactured:

TABLE 4
possible soft gelatin capsule ingredient composition

| ingredient | mg/capsule |
|---|---|
| Compound of formula I | 5 |
| Yellow wax | 8 |
| Hydrogenated Soya bean oil | 8 |
| Partially hydrogenated plant oils | 34 |
| Soya bean oil | 110 |
| Total | 165 |

TABLE 5
possible soft gelatin capsule composition

| ingredient | mg/capsule |
|---|---|
| Gelatin | 75 |
| Glycerol 85% | 32 |
| Karion 83 | 8 (dry matter) |
| Titan dioxide | 0.4 |
| Iron oxide yellow | 1.1 |
| Total | 116.5 |

Manufacturing Procedure

The compound of formula I is dissolved in a warm melting of the other ingredients and the mixture is filled into soft gelatin capsules of appropriate size. The filled soft gelatin capsules are treated according to the usual procedures.

### Example C

Suppositories of the following composition are manufactured:

TABLE 6
possible suppository composition

| ingredient | mg/supp. |
|---|---|
| Compound of formula I | 15 |
| Suppository mass | 1285 |
| Total | 1300 |

Manufacturing Procedure

The suppository mass is melted in a glass or steel vessel, mixed thoroughly and cooled to 45° C. Thereupon, the finely powdered compound of formula I is added thereto and stirred until it has dispersed completely. The mixture is poured into suppository moulds of suitable size, left to cool; the suppositories are then removed from the moulds and packed individually in wax paper or metal foil.

### Example D

Injection solutions of the following composition are manufactured:

TABLE 7
possible injection solution composition

| ingredient | mg/injection solution. |
|---|---|
| Compound of formula I | 3 |
| Polyethylene Glycol 400 | 150 |
| acetic acid | q.s. ad pH 5.0 |
| water for injection solutions | ad 1.0 ml |

Manufacturing Procedure

The compound of formula I is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### Example E

Sachets of the following composition are manufactured:

TABLE 8
possible sachet composition

| ingredient | mg/sachet |
|---|---|
| Compound of formula I | 50 |
| Lactose, fine powder | 1015 |
| Microcrystalline cellulose (AVICEL PH 102) | 1400 |
| Sodium carboxymethyl cellulose | 14 |
| Polyvinylpyrrolidon K 30 | 10 |
| Magnesium stearate | 10 |
| Flavoring additives | 1 |
| Total | 2500 |

Manufacturing Procedure

The compound of formula I is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC or chiral HPLC) or crystallization.

All reaction examples and intermediates were prepared under a nitrogen atmosphere if not specified otherwise.

SMARCA2 HiBiT and SMARCA4 Degradation Assay (Cellular)

Generation of HT1080 Cell Lines Stably Expressing SMARCA2 HiBiT or SMARC4 HiBiT

For quantitative cellular degradation of the target protein degradation mediated by the bi-functional degraders described here, HiBiT was appendant to the gene sequence of the targeted proteins, SMARCA2 or SMARCA4, in HT1080 parental cell line using CRISPR-mediate HiBiT tagging technology, as described by Promega.

RNP Complex Assembly and Delivery. RNA Complexes were assembled and delivered by electroporation into cells, as previously described. Briefly, 16 g (100 pmol) Cas9 and 10.8 g of sgRNA were incubated for 10-15 minutes at room temperature. Cells were resuspended in 20 L of SF 4D-nucleofector solution (Amaxa SF cell line4D Nucleofector X kit (Lonza, V4XC-2032). RNP complex and 16.6 pmol of DNA oligo were the electroporated into cells using FF-113 program (Amaxa 4D Nucleofector). Following electroporation, cells were incubated at room temperature for 5 minutes and then transferred to a six-well plate for culturing. At 24-48 h postelectroporation, cells were analyzed for insertion with Nano-Glo® HiBiT Lytic Detection System.

Lytic HiBiT Detection. Nano-Glo® HiBiT Lytic Detection System was used to assess luminescence for each guide RNA tested (ACS Chem. Biol. 2018, 13, 467-474). Unedited cells were used as negative control for background. Following successful detection of the HiBiT luminescence signal in the pool, the pool of cells was subjected for single cell sorting (SH800S Cell Sorter, Sony Biotechnology). Only clones that gave a highest HiBiT luminescence signal were further expanded in cell culture and were used in the SMARCA2 HiBiT and SMARCA4 HiBiT degradation assay (cellular).

1.1 Materials

SMARCA2 HiBiT and SMARCA4 HiBiT HT1080 cell lines were generated in house as described herein. HT1080 parental cell line, as well as SMARCA2 HiBiT HT1080 and SMARCA4 HiBiT HT1080 cell lines were routinely cultured in the following medium: Earle's MEM (Gibco, #41090) containing 10% serum (VWR, #97068-085) and only up to passage 20. For the assay, SMARCA2 HiBiT HT1080 and SMARCA4 HiBiT HT1080 cells are plated for treatment in Earle's MEM (Gibco, #51200) containing 10% serum (VWR, #97068-085) and 1× Glutamax (Gibco, #35050-038). Assay plates used were Corning® 384-well Flat Clear Bottom White Polystyrene TC-treated Microplates (Corning #3765). Cells for lysed in Nano-Glo® HiBiT Lytic Reagent, Nano-Glo® HiBiT Lytic Detection System, Promega, (#N3050).

1.3 SMARCA2 HiBiT and SMARCA4 HiBiT Degradation Assay (Cellular)

Briefly, a day before compound treatment, cells were seeded onto 384-well plate at the density of 1500 cell/well in Earle's MEM (Gibco, #51200) containing 10% serum (VWR, #97068-085) and 1× Glutamax (Gibco, #35050-038). The following day, test compounds were added to the 384-well plate from a top concentration of 10 μM with 11 points, half log titration in duplicates. Additionally, the negative control cells were treated with vehicle alone. The plates were incubated at 37° C. with 5% CO₂ for duration of the assay (6 hours or 16 hours). After the desired incubation time, cells were lyased by addition of Nano-Glo® HiBiT Lytic Reagent (prepared according the manufacture recommendations and added to the cells in ratio 1:1, v/v). Microplates were agitated on plate shaker at 400 rpm for 2 minutes, and incubated for another 10 min in dark at room temperature. A white light-reflecting film was applied to the bottom of the 384 well plates before reading. Finally, luminescence signal was acquired on with PHERAstar® FSX plate reader (BMG Labtech, Germany).

Quantification of luminescence responses measured in the presence of compound were normalized to a high signal/no degradation control (untreated cells+lytic detection reagent) and a low signal/full degradation control (untreated cells, no lytic detection reagent). Data were analyzed with a 4-parameter logistic fit to generate sigmoidal dose-response curves. The DC₅₀ is the concentration of compound at which exactly 50% of the total cellular SMARCA2 or SMARCA4 has been degraded. The Emax, or maximum effect of each compound, represents the amount of residual protein remaining in the cell following compound treatment.

TABLE 1

| | HiBit A549 | HiBit A549 |
|---|---|---|
| | SMARCA2 | SMARCA2 |
| Example | IP (nM) 6 h | Emax (%) 6 h |
| 1 | 68 | 55 |
| 2 | 550 | 53 |
| 3 | 65 | 50 |
| 4 | 149 | 44 |
| 5 | 66 | 60 |
| 6 | 334 | 58 |
| 7 | 423 | 42 |
| 8 | 254 | 65 |
| 9 | 339 | 58 |
| 10 | 58 | 49 |
| 11 | 146 | 64 |
| 12 | 60 | 55 |
| 13 | 32 | 64 |
| 14 | 31 | 45 |
| 15 | 35 | 52 |
| 16 | 46 | 50 |
| 17 | 1443 | 64 |
| 18 | 134 | 62 |
| 19 | 64 | 34 |
| 20 | 168 | 49 |
| 21 | 118 | 29 |
| 22 | 212 | 28 |
| 23 | 157 | 45 |
| 24 | 256 | 56 |
| 25 | 614 | 59 |
| 26 | 164 | 63 |
| 27 | 333 | 57 |
| 28 | 91 | 48 |
| 29 | 158 | 50 |
| 30 | 92 | 63 |
| 31 | 383 | 55 |
| 32 | 281 | 62 |
| 33 | 15 | 39 |
| 34 | 26 | 24 |
| 35 | 22 | 33 |
| 36 | 16 | 27 |
| 37 | 57 | 34 |
| 38 | 20 | 29 |
| 39 | 718 | 63 |
| 40 | 22 | 32 |
| 41 | 46 | 48 |
| 42 | 519 | 49 |
| 43 | 32 | 31 |
| 44 | 133 | 61 |
| 45 | 627 | 58 |
| 46 | 12 | 24 |
| 47 | 17 | 42 |
| 48 | 15 | 45 |
| 49 | 4 | 39 |
| 50 | 16 | 44 |
| 51 | 86 | 54 |
| 52 | 37 | 52 |
| 53 | 122 | 46 |
| 54 | 42 | 63 |
| 55 | 19 | 35 |
| 56 | 10 | 36 |
| 57 | 406 | 67 |
| 58 | 43 | 59 |
| 59 | 129 | 65 |
| 60 | 18 | 32 |
| 61 | 100 | 53 |
| 62 | 124 | 33 |
| 63 | 59 | 27 |
| 64 | 82 | 52 |
| 65 | 115 | 31 |
| 66 | 144 | 60 |
| 67 | 37 | 54 |
| 68 | 201 | 55 |
| 69 | 16 | 48 |
| 70 | 40 | 42 |
| 71 | 326 | 46 |
| 72 | 273 | 62 |
| 73 | 114 | 49 |
| 74 | 70 | 46 |
| 75 | 1427 | 63 |
| 76 | 221 | 57 |
| 77 | 77 | 57 |
| 78 | 23 | 35 |
| 79 | 19 | 46 |
| 80 | 11 | 51 |
| 81 | 16 | 56 |
| 82 | 190 | 65 |
| 83 | 273 | 59 |
| 84 | 17 | 26 |
| 85 | 92 | 43 |
| 86 | 59 | 37 |
| 87 | 81 | 48 |
| 88 | 55 | 37 |
| 89 | 55 | 30 |
| 90 | 103 | 37 |
| 91 | 90 | 46 |
| 92 | 17 | 41 |
| 93 | 9 | 52 |
| 94 | 18 | 48 |
| 95 | 13 | 25 |
| 96 | 7 | 27 |
| 97 | 659 | 30 |
| 98 | 182 | 63 |
| 99 | 31 | 51 |
| 100 | 104 | 30 |
| 101 | 102 | 28 |
| 102 | 102 | 38 |
| 103 | 104 | 45 |
| 104 | 103 | 33 |
| 105 | 100 | 33 |
| 106 | 23 | 26 |
| 107 | 193 | 51 |
| 108 | 109 | 48 |
| 109 | 103 | 29 |
| 110 | 276 | 52 |
| 111 | 177 | 60 |
| 112 | 101 | 51 |
| 113 | 19 | 35 |
| 114 | 82 | 40 |
| 115 | 810 | 63 |
| 116 | 58 | 32 |
| 117 | 144 | 50 |
| 118 | 11 | 26 |
| 119 | 35 | 50 |
| 120 | 31 | 28 |
| 121 | 47 | 26 |
| 122 | 70 | 29 |
| 123 | 75 | 34 |
| 124 | 272 | 63 |
| 125 | 30 | 42 |
| 126 | 73 | 26 |
| 127 | 4 | 26 |
| 128 | 18 | 37 |
| 129 | 37 | 38 |
| 130 | 12 | 43 |
| 131 | 27 | 32 |
| 132 | 133 | 44 |
| 133 | 64 | 44 |
| 134 | 31 | 36 |
| 135 | 382 | 35 |
| 136 | 495 | 45 |
| 137 | 535 | 63 |
| 138 | 678 | 42 |
| 139 | 802 | 62 |
| 140 | 275 | 42 |
| 141 | 9* | 19* |
| 142 | 2* | 14* |
| 143 | 2* | 8* |
| 144 | 797 | 75 |
| 145 | 928 | 69 |
| 146 | 126* | 41* |
| 147 | 236 | 69 |
| 148 | 25 | 33 |
| 149 | 18 | 40 |
| 150 | 553 | 65 |
| 151 | 855 | 54 |
| 152 | 5* | 42* |
| 153 | 19* | 21* |
| 154 | 12* | 16* |
| 155 | 5* | 26* |
| 156 | 3* | 17* |
| 157 | 108 | 68 |
| 158 | 3* | 5* |
| 159 | 11 | 27 |
| 160 | 144 | 74 |
| 161 | 33 | 30 |
| 162 | 299 | 57 |
| 163 | 22 | 33 |
| 164 | 42 | 50 |
| 165 | 28 | 38 |
| 166 | 607 | 53 |
| 167 | 54 | 43 |
| 168 | 111 | 60 |
| 169 | 49 | 56 |
| 170 | 18 | 31 |
| 171 | 66 | 40 |
| 172 | 24 | 30 |
| 173 | 110 | 72 |
| 174 | 123 | 54 |
| 175 | 42 | 47 |
| 176 | 3* | 31* |
| 177 | 174 | 69 |
| 178 | 187 | 46 |
| 179 | 100 | 39 |
| 181 | 99 | 56 |
| 182 | 103 | 47 |
| 183 | 99 | 37 |
| 184 | 494 | 65 |
| 185 | 241 | 62 |
| 186 | 148 | 42 |
| 187 | 579 | 53 |
| 188 | 193 | 50 |
| 189 | 1370 | 62 |
| 190 | 22 | 29 |
| 191 | 30 | 37 |
| 192 | 57 | 34 |
| 193 | 51 | 25 |
| 194 | 67 | 61 |
| 195 | 72 | 60 |
| 196 | 51 | 65 |
| 197 | 46 | 30 |
| 198 | 277 | 68 |
| 199 | 107 | 62 |
| 200 | 104 | 45 |
| 201 | 79 | 37 |
| 202 | 107 | 44 |
| 203 | 216 | 28 |
| 204 | 907 | 39 |

### Ligase 1: 7-[[2-(2,6-dioxo-3-piperidyl)-1,3-dioxo-isoindolin-4-yl]amino]heptanoic acid

A mixture of 2-(2,6-dioxo-3-piperidyl)-4-fluoro-isoindoline-1,3-dione (100 mg, 0.36 mmol, 1.0 eq), 7-aminoheptanoic acid (79 mg, 0.54 mmol, 1.5 eq) and N,N-diisopropylethylamine (93 mg, 0.72 mmol, 2.0 eq) in DMF (10 mL) was stirred at 80° C. for 12 h. The mixture was concentrated in vacuum, then purified on silica column (heptane/EtOAc 0-100%) (DCM/MeOH 0-100%) to afford the title compound (10 mg, 0.02 mmol, 4% yield) as a yellow oil. MS (ESI): 402.4 ([M+H]+).

### Ligase 2: 3-[1-[2-(2,6-Dioxopiperidin-3-yl)-1,3-dioxoisoindol-4-yl]piperidin-4-yl]propanoic acid

A mixture of 3-(4-piperidyl)propanoic acid hydrochloride (2524 mg, 13 mmol, 1.2 eq) 2-(2,6-dioxo-3-piperidyl)-4-fluoro-isoindoline-1,3-dione (3000 mg, 10.8 mmol, 1 eq), DIPEA (5.68 mL, 32 mmol, 3 eq) in DMSO (50 mL) was stirred at 100° C. for 16 h. The reaction mixture was poured into water (500 mL), the mixture was extracted with EtOAc (200.0 mL×3). The combined organic layers were washed with brine (250.0 mL), dried (Na2SO4) and concentrated. The residue was dissolved in CAN (30 mL) and stirred for 5 min, then a yellow solid was precipitated, filtered and the filter cake was dried under high vacuum to afford the title compound (2300 mg, 5.56 mmol, 49% yield) as a yellow solid. MS (ESI): 414.1 ([M+H+]⁺).

### Ligase 3: 9-[4-[[[2-(2,6-dioxo-3-piperidyl)-1,3-dioxo-isoindolin-4-yl]amino]methyl]triazol-1-yl]nonanoic acid

### a) 2-(2,6-dioxopiperidin-3-yl)-4-(prop-2-yn-1-ylamino)isoindoline-1,3-dione

To a stirred solution of 2-(2,6-dioxo-3-piperidyl)-4-fluoro-isoindoline-1,3-dione (2.5 g, 9.05 mmol) in DMSO (20 mL) was added prop-2-yn-1-amine (747 mg, 13.5 mmol, 869 uL) and DIPEA (5.85 g, 45.2 mmol, 7.88 mL) in a sealed tube. The mixture was heated to 90° C. for 48 h. The reaction was then cooled to room temperature and ice water was added to the reaction mixture, causing a solid precipitate. The yellow solid was collected and purified on silica (Hexane/EtOAc 6:4) to obtain the title compound (1.75 g, 5.5 mmol, 60% yield, 97% purity) as a bright yellow solid.

### b) 9-[4-[[[2-(2,6-dioxo-3-piperidyl)-1,3-dioxo-isoindolin-4-yl]amino]methyl]triazol-1-yl]nonanoic acid

2-(2,6-dioxopiperidin-3-yl)-4-(prop-2-yn-1-ylamino)isoindoline-1,3-dione (0.5 g, 1.53 mmol) and 9-azidononanoic acid (384 mg, 1.83 mmol) were dissolved in DMSO (5 mL) and degassed for 30 mins. Copper(II) sulfate pentahydrate (388 mg, 1.53 mmol) and sodium ascorbate (305 mg, 1.53 mmol) were added and the reaction mixture stirred at room temperature for 2 hr, diluted with EtOAc and brine. The organic layer was separated and dried over sodium sulfate, concentrated under reduced pressure. The crude mixture was purified on silica (DCM/MeOH 1-10%) to get the title compound (0.22 g, 409 umol, 26% yield, 95% purity) as yellow sticky solid. MS (ESI): 511.40 ([M+H]+).

### Ligase 4: 12-[4-[[[2-(2,6-dioxo-3-piperidyl)-1,3-dioxo-isoindolin-4-yl]amino]methyl]triazol-1-yl]dodecanoic acid

To a stirred solution of 2-(2,6-dioxopiperidin-3-yl)-4-(prop-2-yn-1-ylamino)isoindoline-1,3-dione (3 g, 9.64 mmol) in THF (60 mL)/water (15 mL) was added 12-azidododecanoic acid (2.33 g, 9.64 mmol), copper sulfate pentahydrate (2.41 g, 9.64 mmol) and L-ascorbic acid sodium salt (1.91 g, 9.64 mmol). The mixture stirred at room temperature for 2 h, quenched with water (50 ml) and extracted twice with ethyl acetate (250 ml×2). The combined organic layer was washed with brine, dried over sodium sulfate and concentrated under reduced pressure. The crude product was purified by prep-HPLC (FA) to afford the title compound (2.35 g, 4.20 mmol, 43% yield, 98% purity) as yellow solid. MS (ESI): 553.40 ([M+H]⁺).

### Ligase 5: 9-[[2-(2,6-dioxo-3-piperidinyl)-2,3-dihydro-1,3-dioxo-1H-isoindol-4-yl]amino]-nonanoic acid (CAS: 2305936-70-1)

### Ligase 6: 15-[4-[[[2-(2,6-dioxo-3-piperidyl)-1,3-dioxo-isoindolin-4-yl]amino]methyl]triazol-1-yl]pentadecanoic acid

To a stirred solution 2-(2,6-dioxo-3-piperidyl)-4-(prop-2-ynylamino)isoindoline-1,3-dione (2.6 g, 8.35 mmol) and 15-azidopentadecanoic acid (2.37 g, 8.35 mmol) in THF (50 mL) and water (12.5 mL) was added were sodium (L)-ascorbate (1.65 g, 8.35 mmol) and copper sulfate pentahydrate (2.09 g, 8.35 mmol). The mixture was allowed to stir at room temperature for 4 h. Water was added to the reaction mixture. The product was extracted with ethyl acetate (3×50 mL). The combined organic layers were dried over sodium sulfate, filtered and concentrated under reduced pressure. The crude material was purified by prep-HPLC (basic) to afford the title compound (3.0 g, 4.94 mmol, 59% yield, 98% purity) as yellow solid. MS (ESI): 595.51 ([M+H]+).

### Ligase 7: 2-(2,6-Dioxopiperidin-3-yl)-4-(piperidin-4-yloxy)isoindoline-1,3-dione

### a) tert-butyl 4-((2-(2,6-dioxopiperidin-3-yl)-1,3-dioxoisoindolin-4-yl)oxy)piperidine-1-carboxylate

To a solution of 2-(2,6-dioxopiperidin-3-yl)-4-fluoroisoindoline-1,3-dione (500 mg, 1.81 mmol, Eq: 1) and tert-butyl 4-hydroxypiperidine-1-carboxylate (364 mg, 1.81 mmol, Eq: 1) in DMSO (4.5 ml) is added sodium hydride (181 mg, 4.53 mmol, Eq: 2.5) at 0° C. The ice bath is then removed and the reaction is stirred overnight at 90° C. The reaction is poured in 20 mL of 0.5M citric acid yielding a black solution with a dark grey precipitate. The solution is filtred twice, the black solid is dried and used in the next step without further purification (279 mg, 34%).

### b) 2-(2,6-Dioxopiperidin-3-yl)-4-(piperidin-4-yloxy)isoindoline-1,3-dione

To a stirred solution of tert-butyl 4-((2-(2,6-dioxopiperidin-3-yl)-1,3-dioxoisoindolin-4-yl)oxy)piperidine-1-carboxylate (279.2 mg, 610 μmol, Eq: 1) in DCM (4.07 ml) is added HCl 4M in Dioxane (1.53 ml, 6.1 mmol, Eq: 10) and the mixture is stirred at r.t. overnight. The reaction mixture is diluted with 9:1 DCM:MeOH and washed with NaHCO₃ sat., dried over Na₂SO₄, concentrated in vacuo to afford the free base as a brown solid. The crude product is purified on amine modified silica gel (DCM/DCM:MeOH 4:1 0-70) to afford the title compound as a light green solid (37 mg, 17% yield).

### Ligase 8: 2-(2,6-dioxo-3-piperidinyl)-5-(4-piperidinyloxy)-1H-Isoindole-1,3(2H)-dione (CAS: 2222116-10-9)

### Ligase 9: 8-[[2-(2,6-dioxo-3-piperidinyl)-2,3-dihydro-1,3-dioxo-1H-isoindol-4-yl]amino]-octanoic acid (CAS: 2225940-51-0)

### Ligase 10: 6-[[2-(2,6-dioxo-3-piperidinyl)-2,3-dihydro-1,3-dioxo-1H-isoindol-4-yl]amino]-hexanoic acid (CAS: 2225940-49-6)

### Ligase 11: 1-[2-(2,6-dioxo-3-piperidyl)-1,3-dioxo-isoindolin-4-yl]piperidine-4-carboxylic acid

A mixture of isonipecotic acid (1683 mg, 13.0 mmol, 1.2 eq), 2-(2,6-dioxo-3-piperidyl)-4-fluoro-isoindoline-1,3-dione (3000 mg, 10.8 mmol, 1 eq), DIPEA (5.68 mL, 32.5 mmol, 3 eq) in DMSO (50 mL) was stirred at 100° C. for 16 h. Water was added, the mixture was extracted with EtOAc. The combined organic layers were washed with brine, dried over Na2SO4, filtered and concentrated. The residue was purified on silica gel (PE/EtOAc 10-100%) to afford the title compound (560 mg, 1.4 mmol, 12% yield) as an orange solid.

### Ligase 12: 3-[1-[2-(2,6-dioxo-3-piperidyl)-1,3-dioxo-isoindolin-5-yl]-4-piperidyl]propanoic acid

The title compound was pepared in analogy to Ligase 11 using 3-(4-piperidyl)propanoic acid hydrochloride (2.52 g, 13.03 mmol, 1.2 eq). The product was purified by trituration (CH3CN, 60 mL) to afford a yellow solid (82% yield).

### Ligase 13: 2-[1-[2-(2,6-dioxo-3-piperidyl)-1,3-dioxo-isoindolin-4-yl]-4-piperidyl]acetic acid

The title compound was pepared in analogy to Ligase 11 using 2-(4-piperidyl)acetic acid hydrochloride (2.34 g, 13.03 mmol, 1.2 eq). The product was purified by trituration (CH3CN, 60 mL) to afford a yellow solid (52% yield).

### Ligase 14: 4-[[2-[[2-(2,6-dioxo-3-piperidinyl)-2,3-dihydro-1,3-dioxo-1H-isoindol-4-yl]oxy]acetyl]amino]-butanoic acid (CAS: 2308035-51-8)

### Ligase 15: 1-[2-(2,6-dioxo-3-piperidyl)-1,3-dioxo-isoindolin-5-yl]piperidine-4-carboxylic acid

The title compound was pepared in analogy to Ligase 11 using 2-(2,6-dioxo-3-piperidyl)-5-fluoro-isoindoline-1,3-dione (3.0 g, 10.86 mmol, 1 eq) and isonipecotic acid (1.68 g, 13.03 mmol, 1.2 eq). The product was purified by trituration (CH3CN, 60 mL) to afford a green solid (67% yield).

### Ligase 16: 2-(2,6-dioxo-3-piperidinyl)-4-(4-piperidinylamino)-1H-Isoindole-1,3(2H)-dione (CAS: 2154357-05-6)

### Ligase 17: 2-(2,6-dioxo-3-piperidinyl)-4-(methyl-4-piperidinylamino)-1H-Isoindole-1, 3(2H)-dione (CAS: 2154357-11-4)

### Ligase 18: 2-(2,6-dioxo-3-piperidyl)-4-(piperazin-1-ylmethyl)isoindoline-1,3-dione hydrochloride

### a) dimethyl 3-(bromomethyl)benzene-1,2-dicarboxylate

To a solution of dimethyl 3-methylbenzene-1,2-dicarboxylate (6.04 g, 29.01 mmol, 1 eq) and N-bromosuccinimide (5.42 g, 30.46 mmol, 1.05 eq) in acetonitrile (50 mL) was added 2,2′-azobis(2-methylpropionitrile) (0.95 g, 5.8 mmol, 0.200 eq), then the mixture was stirred at 80° C. for 12 h under nitrogen. EtOAc was added and the reaction was washed with water. The organic phase was concentrated under vacuum and purified on silica gel (PE/EtOAc 20-50%) to yield the title compound (10.6 g, 36.9 mmol, 114% yield) as yellow solid.

### b) dimethyl 3-[(4-tert-butoxycarbonylpiperazin-1-yl)methyl]benzene-1,2-dicarboxylate

To a solution of 1-Boc-piperazine (7.14 g, 38.31 mmol, 1.1 eq) in DMSO (100 mL) was DIPEA (18.2 mL, 104 mmol, 3 eq), followed by dimethyl 3-(bromomethyl)benzene-1,2-dicarboxylate (10.0 g, 34 mmol, 1 eq). The mixture was stirred at 90° C. for 3 h, concentrated under vacuum. EtOAc was added and the reaction was washed with water. The organic phase was concentrated under vacuum to the title compound (10 g, 25.4 mmol, 73% yield) as yellow oil.

### c) 3-[(4-tert-butoxycarbonylpiperazin-1-yl)methyl]phthalic acid

To a solution of dimethyl 3-[(4-tert-butoxycarbonylpiperazin-1-yl)methyl]benzene-1,2-dicarboxylate (10.0 g, 25.48 mmol, 1 eq) in THF (80 mL) was added sodium hydroxide (10.19 g, 254.8 mmol, 10 eq) and water (20 mL). The mixture was stirred at 50° C. for 3 h. The pH was adjusted to 4-5 with 0.5M HCl, and the mixture was concentrated in vacuo to yield the title compound (9 g, 24.7 mmol, 91% yield) as white solid.

### d) tert-butyl 4-[[2-(2,6-dioxo-3-piperidyl)-1,3-dioxo-isoindolin-4-yl]methyl]piperazine-1-carboxylate

To a solution of 3-[(4-tert-butoxycarbonylpiperazin-1-yl)methyl]phthalic acid hydrochloride (4.5 g, 11.2 mmol, 1 eq) in pyridine (45 mL) was added 3-aminopiperidine-2,6-dione (1.86 g, 11.2 mmol, 1 eq). The mixture was strried at 80° C. for 2 h. The reaction mixture was concentrated under vacuum. The residue was purified by pre-HPLC (FA) to yield the title compound (1.3 g, 2.85 mmol, 24% yield) as grey solid.

### e) 2-(2,6-dioxo-3-piperidyl)-4-(piperazin-1-ylmethyl)isoindoline-1,3-dione hydrochloride

To a solution of tert-butyl 4-[[2-(2,6-dioxo-3-piperidyl)-1,3-dioxo-isoindolin-4-yl]methyl]piperazine-1-carboxylate (1.2 g, 2.63 mmol, 1 eq) in DCM (30 mL) was added 4N HCl in EtOAc (30 mL, 2.63 mmol, 1 eq) at 0° C. The mixture was stirred at 0° C. for 2 h, concentrated under vacuum at 10° C. to yield the title compound (970 mg, 2.47 mmol, 93% yield) as grey solid.

### Ligase 19: 14-[4-[4-[(2,6-dioxo-3-piperidyl)amino]phenyl]piperazin-1-yl]-14-oxo-tetradecanoic acid

### a) tert-butyl 4-(4-((2,6-dioxopiperidin-3-yl)amino)phenyl)piperazine-1-carboxylate

tert-Butyl 4-(4-aminophenyl)piperazine-1-carboxylate (2.5 g, 9.0 mmol) and 3-bromopiperidine-2,6-dione (2.94 g, 15.3 mmol) were dissolved in DMF (20 mL). To this was added sodium bicarbonate (2.27 g, 27.04 mmol, 1.05 mL) at room temperature. The resulted mixture was stirred at 80° C. for 24 h. The reaction mixture was then cooled to room temperature and diluted with water (80 mL) and stirred for half an hour. A precipitate formed and was collected via filtration. The crude material was purified on silica (PE/EtOAc 0-100) to yield the title compound (2.1 g, 4.8 mmol, 53% yield, 89% purity) as grey solid. MS (ESI): 389.0 ([M+H]⁺).

### b) 3-((4-(piperazin-1-yl)phenyl)amino)piperidine-2,6-dione

tert-Butyl 4-[4-[(2,6-dioxo-3-piperidyl)amino]phenyl]piperazine-1-carboxylate (4.2 g, 10.8 mmol) was dissolved in DCM (15 mL). To this was added 4.5M HCl in 1,4-dioxane (10.8 mmol, 15 mL) at 0° C. and the resulting mixture was stirred at room temperature for 1 hr. The reaction mixture was concentrated under reduced pressure to yield the title compound (3.8 g, 8.70 mmol, 80% yield) as grey color solid. MS (ESI): 289.0 ([M+H]⁺).

### c) tert-butyl 14-(4-(4-((2,6-dioxopiperidin-3-yl)amino)phenyl)piperazin-1-yl)-14-oxotetradecanoate

To a stirred solution of 3-(4-piperazin-1-ylanilino)piperidine-2,6-dione (1.0 g, 1.59 mmol) and 14-tert-butoxy-14-oxo-tetradecanoic acid (498 mg, 1.59 mmol) in DMF (15 mL) was added DIPEA (1.23 g, 9.52 mmol, 1.66 mL). The reaction mixture was stirred for 5 min at room temperature. PyBOP (990 mg, 1.90 mmol) was added and the reaction mixture was stirred for 16 h. DMF was evaporated using a Genevac and the crude residue was purified by prep-HPLC to afford the title compound (0.7 g, 849 umol, 54% yield) as a yellow solid. MS (ESI): 585.3 ([M+H]⁺).

### d) 14-(4-(4-((2,6-dioxopiperidin-3-yl)amino)phenyl)piperazin-1-yl)-14-oxotetradecanoic acid, 2,2,2-trifluoroacetate salt

To a stirred solution of tert-butyl 14-[4-[4-[(2,6-dioxo-3-piperidyl)amino]phenyl]piperazin-1-yl]-14-oxo-tetradecanoate (0.25 g, 427 umol) in DCM (10 mL) was added trifluoroacetic acid (974 mg, 8.55 mmol, 658 uL) at room temperature. The mixture was stirred at room temperature for 3 h, concentrated under reduced pressure. The crude compound was triturated with diethyl ether to the title compound (200 mg, 253 umol, 59% yield, 96% purity) as a grey solid. MS (ESI): 527.3 ([M−H]⁻).

### Ligase 20: 9-[4-[2-(2,6-dioxo-3-piperidyl)-1,3-dioxo-isoindolin-5-yl]piperazin-1-yl]nonanoic acid

### a) tert-butyl 4-(2-(2,6-dioxopiperidin-3-yl)-1,3-dioxoisoindolin-5-yl)piperazine-1-carboxylate

To a solution of 2-(2,6-dioxo-3-piperidyl)-5-fluoro-isoindoline-1,3-dione (0.1 g, 362 umol) in dimethylacetamide (1.0 mL) were added tert-butyl piperazine-1-carboxylate (67 mg, 362 umol) followed by DIPEA (116 mg, 905 umol, 157 uL) at room temperature under nitorgen atmosphere. The reaction mixture was heated at 90° C. for 12 h. Reaction mixture was cooled to room temperature, added to water, resulting solid was filtered and dried to afford the title compound (25.0 mg, 56.5 umol, 15% yield) as a yellow solid. MS (ESI): 386.9 ([M-57+H]⁺).

### b) 2-(2,6-dioxopiperidin-3-yl)-5-(piperazin-1-yl)isoindoline-1,3-dione

To a solution of tert-butyl 4-[2-(2,6-dioxo-3-piperidyl)-1,3-dioxo-isoindolin-5-yl]piperazine-1-carboxylate (25.0 mg, 56.5 umol) in 1,4-dioxane (0.25 mL) was added 4 M HCl in dioxane (0.25 g, 56.5 umol) at 0° C. The reaction mixture was warmed to room temperature and stirred for 2 h. Reaction mixture was concentrated under reduced pressure, and co-distilled with dichloromethane to afford the title compound (20.0 mg, 58 umol, 103% yield) as a light brown semi solid, HCl salt. MS (ESI): 343.4 ([M+H]⁺).

### c) methyl 9-(4-(2-(2,6-dioxopiperidin-3-yl)-1,3-dioxoisoindolin-5-yl)piperazin-1-yl)nonanoate

To a solution of 2-(2,6-dioxo-3-piperidyl)-5-piperazin-1-yl-isoindoline-1,3-dione (0.27 g, 788 umol) in dimethylformamide (3 mL) were added triethylamine (399 mg, 3.94 mmol, 549 uL) followed by methyl 9-bromononanoate (217 mg, 867 umol) at room temperature under nitrogen atmosphere. The reaction mixture was heated at 80° C. for 16 h. Reaction mixture was cooled to room temperature, added to water and extracted with EtOAc. The organic layer was washed with water, brine solution, dried over anhydrous sodium sulfate, filtered and concentrated under reduced pressure. The crude residue was purified on silica (PE/EtOAc 0-60%) to afford the title compound (0.11 g, 214 umol, 27% yield) as a brown liquid. MS (ESI): 513.8 ([M+H]⁺).

### d) 9-(4-(2-(2,6-dioxopiperidin-3-yl)-1,3-dioxoisoindolin-5-yl)piperazin-1-yl)nonanoic acid

To a solution of methyl 9-[4-[2-(2,6-dioxo-3-piperidyl)-1,3-dioxo-isoindolin-5-yl]piperazin-1-yl]nonanoate (0.11 g, 214 umol) in DCM (1.1 mL) was added trimethyltinhydroxide (193 mg, 1.07 mmol) at room temperature. The reaction mxiture was heated at 80° C. for 16 h. Reaction mixture was cooled to room temperature, concentrated under reduced pressure. The crude residue was purified by prep-HPLC to afford the title compound (0.04 g, 80 umol, 37% yield) as a yellow semisolid. MS (ESI): 499.3 ([M+H]⁺).

### Ligase 21: 10-[4-[[[2-(2,6-dioxo-3-piperidyl)-1,3-dioxo-isoindolin-4-yl]amino]methyl]triazol-1-yl]decanoic acid

To a mixture of 10-azidodecanoic acid (110 mg, 515 umol) and 2-(2,6-dioxo-3-piperidyl)-4-(prop-2-ynylamino)isoindoline-1,3-dione (160 mg, 515 umol) in THF (3 mL) and water (0.3 mL), were added sodium ascorbate (102 mg, 515 umol) followed by copper sulfate (138 mg, 515 umol) at room temperature. The resulitng mixture was stirred at room temperature for 16 h, dilited with water and extracted with EtOAc. The organic layer was washed with water, brine solution, dried over sodium sulfate, filtered and concentrated under reduced pressure. The crude residue was purified on silica (DCM/MeOH 0-15%) to afford the title compound (65 mg, 123 umol, 24% yield) as a light yellow solid. MS (ESI): 525.2 ([M+H]⁺).

### Ligase 22: 11-[4-[[[2-(2,6-dioxo-3-piperidyl)-1,3-dioxo-isoindolin-4-yl]amino]methyl]triazol-1-yl]undecanoic acid

To a mixture of 11-azidoundecanoic acid (95.0 mg, 417 umol) and 2-(2,6-dioxo-3-piperidyl)-4-(prop-2-ynylamino)isoindoline-1,3-dione (130 mg, 417 umol) in THF (3 mL) and water (0.3 mL), were added sodium ascorbate (165 mg, 835 umol) followed by copper sulfate (223 mg, 835 umol) at room temperature. The reaction mixture was stirred at room temperature for 16 h, diluted with water and extracted with ethyl acetate. The organic layer was washed with water, brine solution, dried over sodium sulfate, filtered and concentrated under reduced pressure. The crude residue was purified on silica (DCM/MeOH 0-10%) to afford the title compound (60 mg, 111 umol, 26% yield) as a light yellow solid. MS (ESI): 539.2 ([M+H]⁺).

### Ligase 23: 10-[[2-(2,6-dioxo-3-piperidinyl)-2,3-dihydro-1,3-dioxo-1H-isoindol-4-yl]amino]-decanoic acid (CAS: 2243000-24-8)

### Ligase 24: 2-[1-[2-(2,6-Dioxopiperidin-3-yl)-1,3-dioxoisoindol-5-yl]piperidin-4-yl]acetic acid

The title compound was pepared in analogy to Ligase 11 using from 2-(2,6-dioxo-3-piperidyl)-5-fluoro-isoindoline-1,3-dione and 2-(4-piperidyl)acetic acid hydrochloride. The product was purified by trituration (CH3CN, 60 mL) to afford a yellow solid (47% yield). MS ESI: 400.3 ([M+H+]⁺).

### Ligase 25: 5-[4-(2-bromoethyl)-1-piperidyl]-2-(2,6-dioxo-3-piperidyl)isoindoline-1,3-dione

### a) 2-(2,6-dioxo-3-piperidyl)-4-[4-(2-hydroxyethyl)-1-piperidyl]isoindoline-1,3-dione

The title compound was pepared in analogy to Ligase 11 using 2-(2,6-dioxo-3-piperidyl)-5-fluoro-isoindoline-1,3-dione (3.0 g, 10.86 mmol, 1 eq) and 4-piperidineethanol (1.68 g, 13.03 mmol, 1.2 eq). The product was purified on amine modified silica gel to afford a yellow oil (39% yield).

### b) 5-[4-(2-bromoethyl)-1-piperidyl]-2-(2,6-dioxo-3-piperidyl)isoindoline-1,3-dione

To the mixture of 2-(2,6-dioxo-3-piperidyl)-4-[4-(2-hydroxyethyl)-1-piperidyl]isoindoline-1,3-dione (1600 mg, 4.15 mmol, 1 eq) in THF (1 mL) was added carbon tetrabromide (2753 mg, 8.3 mmol, 2 eq), triphenylphosphine (2177 mg, 8.3 mmol, 2 eq). The mixture was stirred at 25° C. for 12 h. The mixture was concentrated, purified by prep-HPLC to give the title compound (600 mg, 1.34 mmol, 58% yield) as yellow solid.

### Ligase 26: 5-[4-(2-bromoethoxy)-1-piperidyl]-2-(2,6-dioxo-3-piperidyl)isoindoline-1,3-dione

### a) 2-(2,6-dioxo-3-piperidyl)-5-[4-(2-hydroxyethoxy)-1-piperidyl]isoindoline-1,3-dione

The title compound was pepared in analogy to Ligase 11 using 2-(2,6-dioxo-3-piperidyl)-5-fluoro-isoindoline-1,3-dione and 2-(4-piperidyloxy)ethanol. The product was purified on amine modified silica gel to afford a yellow solid (38% yield).

### b) 5-[4-(2-bromoethoxy)-1-piperidyl]-2-(2,6-dioxo-3-piperidyl)isoindoline-1,3-dione

To a solution of 2-(2,6-dioxo-3-piperidyl)-5-[4-(2-hydroxyethoxy)-1-piperidyl]isoindoline-1,3-dione (1.0 g, 2.49 mmol, 1 eq) in DCM (20 mL) under 25° C. was added carbon tetrabromide (1.65 g, 4.98 mmol, 2 eq), after triphenylphosphine (1.31 g, 4.98 mmol, 2 eq) was added and stirred at 25° C. for 3 h. The solution was concentrated and purified on silica gel (PE/EtOAc 1:1), Prep-HPLC (neutral) to give the title compound (503 mg, 1.08 mmol, 40% yield) as yellow solid.

### Ligase 27: 5-[4-(3-bromopropoxy)-1-piperidyl]-2-(2,6-dioxo-3-piperidyl)isoindoline-1,3-dione

### a) 2-(2,6-dioxo-3-piperidyl)-5-[4-(3-hydroxypropoxy)-1-piperidyl]isoindoline-1,3-dione

The title compound was pepared in analogy to Ligase 11 using 2-(2,6-dioxo-3-piperidyl)-5-fluoro-isoindoline-1,3-dione and 3-(4-piperidyloxy)propan-1-ol. The product was purified on amine modified silica gel to afford a yellow oil (61% yield).

### b) 5-[4-(3-bromopropoxy)-1-piperidyl]-2-(2,6-dioxo-3-piperidyl)isoindoline-1,3-dione

To a solution of 2-(2,6-dioxo-3-piperidyl)-5-[4-(3-hydroxypropoxy)-1-piperidyl]isoindoline-1,3-dione (2.1 g, 5.05 mmol, 1 eq) in DCM (30 mL) was added carbon tetrabromide (3.35 g, 10.11 mmol, 2 eq), after triphenylphosphine (2.65 g, 10.11 mmol, 2 eq) was added to the solution and stirred at 25° C. for 2 h. The solution was concentrated and purified by Prep-HPLC (neutral) to give the title compound (659 mg, 1.38 mmol, 26% yield) as yellow solid.

### Ligase 28: 9-[4-[[[2-(2,6-dioxo-3-piperidyl)-1,3-dioxo-isoindolin-4-yl]amino]methyl]triazol-1-yl]nonanal

### a) 2-(2,6-dioxopiperidin-3-yl)-4-(((1-(9-hydroxynonyl)-1H-1,2,3-triazol-4-yl)methyl)amino)isoindoline-1,3-dione

To a mixture of 2-(2,6-dioxo-3-piperidyl)-4-(prop-2-ynylamino)isoindoline-1,3-dione (800 mg, 2.57 mmol) and 9-azidononan-1-ol (476 mg, 2.57 mmol) in THF (8 mL) and water (1.5 mL) were added copper sulfate (820 mg, 5.14 mmol, 227 uL) followed by sodium ascorbate (1.02 g, 5.14 mmol) at room temperature. The resulitng mixture was stirred at room temperature for 16 h, diluted with water and extracted with EtOAc. The organic layer was washed with brine solution, dried over sodium sulfate, filtered and concentrated under reduced pressure. The crude residue was on silica (DCM/MeOH 0-15%) to afford the title compound (390 mg, 785 umol, 30% yield) as a pale yellow solid. MS (ESI): 497.0 ([M+H]⁺).

### b) 9-[4-[[[2-(2,6-dioxo-3-piperidyl)-1,3-dioxo-isoindolin-4-yl]amino]methyl]triazol-1-yl]nonanal

To a solution of 2-(2,6-dioxo-3-piperidyl)-4-[[1-(9-hydroxynonyl)triazol-4-yl]methylamino]isoindoline-1,3-dione (390 mg, 785 umol) in DCM (6 mL) was added Dess-Martin Periodinane (499 mg, 1.18 mmol) at 0° C. under nitrogen atmosphere. The reaction mixture was warmed to room temperature and stirred for 3 h, filtered through celite pad and washed with dichloromethane. Resulting filtrate was washed with brine solution, dried over sodium sulfate, filtered and concentrated under reduced pressure to afford title compound (350 mg, 636 umol, 81% yield) as an off white semi solid. MS (ESI): 494.9 ([M+H]⁺).

### Ligase 29: 9-[4-[1-[[2-(2,6-dioxo-3-piperidyl)-1,3-dioxo-isoindolin-4-yl]amino]-1-methyl-ethyl]triazol-1yl]nonanal

### a) 2-(2,6-dioxopiperidin-3-yl)-4-((2-methylbut-3-yn-2-yl)amino)isoindoline-1,3-dione

To a solution of 2-(2,6-dioxo-3-piperidyl)-4-fluoro-isoindoline-1,3-dione (0.5 g, 1.81 mmol) in DMSO (5 mL) were added DIPEA (701 mg, 5.43 mmol, 945 uL) followed by 2-methylbut-3-yn-2-amine (376 mg, 4.53 mmol, 476 uL) at room temperature under nitrogen atmosphere. The reaction mixture was heated at 80° C. for 16 h. Reaction mixture was cooled to room temperature, added to water. The resulitng solid was filtered, washed with water and dried to afford title compound (0.22 g, 36%) as an off-white solid. MS (ESI): 339.9 ([M+H]⁺).

### b) 2-(2,6-dioxopiperidin-3-yl)-4-((2-(1-(9-hydroxynonyl)-1H-1,2,3-triazol-4-yl)propan-2-yl)amino)isoindoline-1,3-dione

To a solution of 4-(1,1-dimethylprop-2-ynylamino)-2-(2,6-dioxo-3-piperidyl)isoindoline-1,3-dione (0.22 g, 648 umol) and 9-azidononan-1-ol (120 mg, 648 umol) in THF (6 mL) and water (1.5 mL) were added, copper sulfate (206 mg, 1.30 mmol, 57 uL) followed by sodium ascorbate (256 mg, 1.30 mmol) at room temperature. The reaction mixture was stirred at room temperature for 16 h, diluted with EtOAc, washed with water and brine solution, dried over sodium sulfate, filtered and concentrated under reduced pressure to afford title compound (0.12 g, 228 umol, 35% yield) as a light yellow solid. MS (ESI): 522.69 ([M−H]⁻).

### c) 9-[4-[1-[[2-(2,6-dioxo-3-piperidyl)-1,3-dioxo-isoindolin-4-yl]amino]-1-methyl-ethyl]triazol-1yl]nonanal

To a solution of 2-(2,6-dioxo-3-piperidyl)-4-[[1-[1-(9-hydroxynonyl)triazol-4-yl]-1-methyl-ethyl]amino]isoindoline-1,3-dione (0.12 g, 228 umol) in DCM (5 mL) was added Dess-Martin Periodinane (145 mg, 343 umol) at 0° C. under nitrogen atmosphere. The reaction mixture was warmed to room temperature and stirred for 1 hour. Reaction mixture was filtered through celite pad and washed with dichloromethane. Resulting filtrate was washed with brine solution, dried over sodium sulfate, filtered and concentrated under reduced pressure. The crude residue was purified on silica (DCM/MeOH 0-5%) to afford title compound (0.06 g, 114 umol, 50% yield) as a light yellow solid. MS (ESI): 521.3 ([M−H]⁻).

### Ligase 30: 9-[4-[1-[[2-(2,6-dioxo-3-piperidyl)-1,3-dioxo-isoindolin-4-yl]amino]ethyl]triazol-1-yl]nonanal

### a) 4-(but-3-yn-2-ylamino)-2-(2,6-dioxopiperidin-3-yl)isoindoline-1,3-dione

To a solution of 2-(2,6-dioxo-3-piperidyl)-4-fluoro-isoindoline-1,3-dione (1.50 g, 5.43 mmol) in DMSO (15.0 mL) were added DIPEA (1.75 g, 13.5 mmol, 2.36 mL) and but-3-yn-2-amine (562 mg, 8.15 mmol, 521 uL) at room temperature under nitrogen atmosphere. The reaction mixture was heated at 80° C. for 24 h. Reaction mixture was cooled to room temperature, added to water, resulting solid was filtered, washed with water and dried to afford title compound (880 mg, 2.71 mmol, 49% yield) as a light yellow soild. MS (ESI): 326.3 ([M+H]⁺).

### b) 2-(2,6-dioxopiperidin-3-yl)-4-((1-(1-(9-hydroxynonyl)-1H-1,2,3-triazol-4-yl)ethyl)amino)isoindoline-1,3-dione

To a mixture of 2-(2,6-dioxo-3-piperidyl)-4-(1-methylprop-2-ynylamino)isoindoline-1,3-dione (880 mg, 2.71 mmol) and 9-azidononan-1-ol (501 mg, 2.71 mmol) in DMSO (3.0 mL) were added sodium ascorbate (32.15 mg, 162 umol) and copper sulfate (8.64 mg, 54.1 umol) in water (0.3 mL) at room temperature. The reaction mixture was stirred at room temperature for 3 h. Reaction mixture was added to water and extracted with 5% methanol in dichloromethane. The organic layer was washed with water, brine solution, dried over sodium sulfate, filtered and concentrated under reduced pressure. The crude residue was purified on silica (DCM/MeOH 0-15%) to afford the title compound (780 mg, 1.53 mmol, 56% yield) as a light yellow semi solid. MS (ESI): 511.1 ([M+H]⁺).

### c) 9-[4-[1-[[2-(2,6-dioxo-3-piperidyl)-1,3-dioxo-isoindolin-4-yl]amino]ethyl]triazol-1-yl]nonanal

To a solution of 2-(2,6-dioxo-3-piperidyl)-4-[1-[1-(9-hydroxynonyl)triazol-4-yl]ethylamino]isoindoline-1,3-dione (300 mg, 587 umol) in DCM (6.0 mL) was added pyridinium chlorochromate (189 mg, 881 umol) at 0° C. under nitrogen atmosphere. The reaction mixture was warmed to room temperature and stirred for 3 h. Reaction mixture was filtered through celite bed and washed with DCM. Resulitng filtrate was washed with water, brine solution, dried over sodium sulfate, filtered and concentrated under reduced pressure to afford tiltle compound (190 mg, 272 umol, 46% yield) as a light yellow semi solid. MS (ESI): 509.3 ([M+H]⁺).

### Ligase 31: 9-[4-[1-[[2-(2,6-dioxo-3-piperidyl)-1,3-dioxo-isoindolin-4-yl]amino]cyclopropyl]triazol-1-yl]nonanal

### a) 2-(2,6-dioxopiperidin-3-yl)-4-((1-ethynylcyclopropyl)amino)isoindoline-13-dione

To a mixture of 2-(2,6-dioxo-3-piperidyl)-4-fluoro-isoindoline-1,3-dione (600 mg, 2.17 mmol) and 1-ethynylcyclopropanamine (306 mg, 2.61 mmol, HCl-salt) in N-methyl-2-pyrrolidone (5.0 mL) was added DIPEA (1.40 g, 10.86 mmol, 1.89 mL) at room temperature under nitrogen atmosphere. The reaction mixture was heated at 80° C. for 24 h. Reaction mixture was cooled to room temperature, added to water, resulting solid was filtered, washed with diethyl ether and dried to afford title compound (680 mg, 201 umol, 9% yield) as a light yellow solid. MS (ESI): 338.3 ([M+H]⁺).

### b) 2-(2,6-dioxopiperidin-3-yl)-4-((1-(1-(9-hydroxynonyl)-1H-1,2,3-triazol-4-yl)cyclopropyl)amino)isoindoline-1,3-dione

To a mixture of 9-azidononan-1-ol (37.0 mg, 199 umol) and 2-(2,6-dioxo-3-piperidyl)-4-[(1-ethynylcyclopropyl)amino]isoindoline-1,3-dione (673 mg, 199 umol) in THF (4.0 mL) and water (0.6 mL) were added sodium ascorbate (79.13 mg, 399 umol) followed by copper sulfate (106 mg, 399 umol) at room temperature. The reaction mixture was stirred at room temperature for 16 h. Reaction mixture was diluted with water, extracted with ethyl acetate. The organic layer was washed with water, brine solution, dried over sodium sulfate, filtered and concentrated under reduced pressure. The crude residue was purified on silica (DCM/MeOH 0-15%) to afford the title compound (220 mg, 101 umol, 50% yield) as a pale yellow solid. MS (ESI): 522.9 ([M+H]⁺).

### c) 9-[4-[1-[[2-(2,6-dioxo-3-piperidyl)-1,3-dioxo-isoindolin-4-yl]amino]cyclopropyl]triazol-1-yl]nonanal

To a solution of 2-(2,6-dioxo-3-piperidyl)-4-[[1-[1-(9-hydroxynonyl)triazol-4-yl]cyclopropyl]amino]isoindoline-1,3-dione (220 mg, 101 umol) in DCM (5.0 mL) was added Dess-Martin Periodinane (64 mg, 151 umol) at 0° C. under nitrogen atmosphere. The resulting mixture was warmed to room temperature and stirred for 3 h. Reaction mixture was filtered through celite bed, washed with DCM, resulting filtrate was washed with brine solution, dried over sodium sulfate, filtered and concentrated under reduced pressure. The crude residue was purified on silica (DCM/MeOH 0-15%) to afford the title compound (120 mg, 87 umol, 86% yield) as a pale yellow solid. MS (ESI): 521.0 ([M+H]⁺).

### Ligase 32: 9-[4-[1-[[2-(2,6-dioxo-3-piperidyl)-1,3-dioxo-isoindolin-4-yl]amino]propyl]triazol-1-yl]nonanal

### a) 2-(2,6-dioxopiperidin-3-yl)-4-(pent-1-yn-3-ylamino)isoindoline-1,3-dione

To a solution of 2-(2,6-dioxo-3-piperidyl)-4-fluoro-isoindoline-1,3-dione (500 mg, 1.81 mmol) in DMSO (5.0 mL) were added DIPEA (701 mg, 5.43 mmol, 945 uL) followed by pent-1-yn-3-amine (300 mg, 3.62 mmol) at room temperature under nitrogen atmosphere. The reaction mixture was heated at 80° C. for 16 h. Reaction mixture was cooled to room temperature added to water, resulting solid was filtered and dried to afford title compound (210 mg, 446 umol, 24% yield) as a pale yellow solid. MS (ESI): 340.0 ([M+H]⁺).

### b) 2-(2,6-dioxopiperidin-3-yl)-4-((1-(1-(9-hydroxynonyl)-1H-1,2,3-triazol-4-yl)propyl)amino)isoindoline-1,3-dione

To a mixture of 2-(2,6-dioxo-3-piperidyl)-4-(1-ethylprop-2-ynylamino)isoindoline-1,3-dione (0.21 g, 618 umol) and 9-azidononan-1-ol (114 mg, 618 umol) in DMSO (2.0 mL) were added copper sulfate (29.6 mg, 185 umol, 8.23 uL) and sodium ascorbate (12.2 mg, 61.8 umol) in water (0.5 mL) at room temperature. The reaction mixture was stirred at room temperature for 2 h. Reaction mixture was diluted with water and extracted with ethyl acetate. The organic layer was washed with water, brine solution, dried over sodium sulfate, filtered and concentrated under reduced pressure. The crude residue was purified on silica (PE/EtOAc 0-100%) to afford the title compound (0.18 g, 328 umol, 53% yield) as a light yellow solid. MS (ESI): 525.2 ([M+H]⁺).

### c) 9-[4-[1-[[2-(2,6-dioxo-3-piperidyl)-1,3-dioxo-isoindolin-4-yl]amino]propyl]triazol-1-yl]nonanal

To a solution of 2-(2,6-dioxo-3-piperidyl)-4-[1-[1-(9-hydroxynonyl)triazol-4-yl]propylamino]isoindoline-1,3-dione (0.18 g, 343 umol) in DCM (5.0 mL) was added Dess-Martin Periodinane (218 mg, 514 umol) at 0° c. under nitrogen atmosphere. The reaction mixture was stirred at room temperature for 16 h. Reaction mixture was filtered through celite bed, washed with dichloromethane. The resulitng filtrate was washed with water, brine solution, dried over sodium sulfate, filtered and concentrated under reduced pressure to afford title compound (80 mg, 56.9 umol, 37% yield) as a light yellow solid. MS (ESI): 523.1 ([M+H]⁺).

### Ligase 33: 5-[4-(Bromomethyl)-1-piperidyl]-2-[(3RS)-2,6-dioxo-3-piperidyl]isoindoline-1,3-dione

To the mixture of 2-(2,6-dioxo-3-piperidyl)-4-[4-(hydroxymethyl)-1-piperidyl]isoindoline-1,3-dione (1.97 g, 5.31 mmol, 1 eq, CAS: 2229717-49-9) in THF (20 mL) was added carbon tetrabromide (3.52 g, 10.62 mmol, 2 eq), triphenylphosphine (2.79 g, 10.62 mmol, 2 eq). The mixture was stirred at 25° C. for 12 h. The mixture was concentrated in vacuo. The crude product was purified by prep-HPLC to give the title compound (853 mg, 1.96 mmol, 36% yield) as a yellow solid.

### Ligase 34: 9-[4-[2-(2,6-dioxo-3-piperidyl)-1,3-dioxo-isoindolin-5-yl]oxy-1-piperidyl]nonanoic acid

### a) 4-((1-(tert-butoxycarbonyl)piperidin-4-yl)oxy)phthalic acid

To a mixture of 5-fluoroisobenzofuran-1,3-dione (500 mg, 3.01 mmol) and tert-butyl 4-hydroxypiperidine-1-carboxylate (666 mg, 3.31 mmol) in DMSO (9.0 mL) was added sodium hydride (60% in oil dispersion) (138.4 mg, 6.02 mmol) in portions at 0° C. followed by DIPEA (778 mg, 6.02 mmol, 1.05 mL). The reaction mixture was heated at 90° C. for 2 h. Reaction mixture was cooled to room temperature, quenched with aqueous ammonium chloride solution and extracted with EtOAc. The organic layer was washed with water, brine solution, dried over sodium sulfate, filtered and concentrated under reduced pressure to afford the title compound (300 mg, 683 umol, 22% yield) as an off-white solid, MS (ESI): 364.0 ([M−H]⁻).

### b) tert-butyl 4-((1,3-dioxo-1,3-dihydroisobenzofuran-5-yl)oxy)piperidine-1-carboxylate

A mixture of 4-[(1-tert-butoxycarbonyl-4-piperidyl)oxy]phthalic acid (300 mg, 821 umol) and acetyl acetate (83.8 mg, 821 umol, 77 uL) in DCM (5.0 mL) was heated at 50° C. for 4 h. Reaction mixture was cooled to room temperature and concentrated under reduced pressure. The crude residue was purified on silica (PE/EtOAc 0-100%) to afford the title compound (55.0 mg, 142 umol, 17% yield) as an off-white solid. MS (ESI): 525.2 ([M+H]⁺).

### c) 2-(2,6-dioxopiperidin-3-yl)-5-(piperidin-4-yloxy)isoindoline-1,3-dione

A mixture of tert-butyl 4-(1,3-dioxoisobenzofuran-5-yl)oxypiperidine-1-carboxylate (50.14 mg, 144 umol), 3-aminopiperidine-2,6-dione (23.76 mg, 144 umol, HCl-salt) and sodium acetate (29.6 mg, 360 umol, 19.35 uL) in acetic acid (0.5 mL) was heated in a sealed tube at 100° C. for 16 h. Reaction mixture was cooled to room temperature and concentrated under reduced pressure. The crude compound was dissolved in ethyl acteate and washed with aqueous sodium bicarbonate solution. The organic layer was washed with water, brine solution, dried over sodium sulfate, filtered and concentrated under reduced pressure. The crude residue was purified on silica (PE/EtOAc 0-100%) to afford the title compound (43.0 mg, 120 umol, 83% yield) as a an off white semi solid. MS (ESI): 358.1 ([M+H]⁺).

### d) 9-[4-[2-(2,6-dioxo-3-piperidyl)-1,3-dioxo-isoindolin-5-yl]oxy-1-piperidyl]nonanoic acid

To a mixture of 2-(2,6-dioxo-3-piperidyl)-5-(4-piperidyloxy)isoindoline-1,3-dione (43.0 mg, 120 umol) and 9-bromononanoic acid (31.39 mg, 132 umol) in DMF (0.8 mL) was added DIPEA (46.6 mg, 360 umol, 62.8 uL) at room temperature under nitrogen atmosphere. The reaction mixture was heated at 80° C. for 16 h. Reaction mixture was cooled to room temperature, diluted with water and extracted with ethyl acetate. The organic layer was washed with water, brine solution, dried over sodium sulfate, filtered and concentrated under reduced pressure. The crude residue was purified on silica (DCM/MeOH 0-10%) to afford the title compound (19.0 mg, 37 umol, 30% yield) as an off white solid. MS (ESI): 514.2 ([M+H]⁺).

### Ligase 35: 2-[4-[4-[(2,6-dioxo-3-piperidyl)oxy]phenyl]-1-piperidyl]acetic acid hydrochloride

### a) tert-butyl 2-(4-(4-((2,6-dioxopiperidin-3-yl)oxy)phenyl)piperidin-1-yl)acetate

To Ligase 38 (500 mg, 1.54 mmol, Eq: 1) in DMF (5.13 ml) was added tert-butyl 2-bromoacetate (300 mg, 1.54 mmol, Eq: 1) and DIPEA (796 mg, 1.08 ml, 6.16 mmol, Eq: 4). The reaction was heated at 60° C. 1 h, partionated between water and EtOAc. The aqueous layer was extracted with EtOAc. The organic layers were combined, washed with sat NaCl, dried over Na2SO4, filtered and concentrated in vacuo to give the title compund (699 mg, 90%) as yellow solid.

### b) 2-[4-[4-[(2,6-dioxo-3-piperidyl)oxy]phenyl]-1-piperidyl]acetic acid hydrochloride

To tert-butyl 2-(4-(4-((2,6-dioxopiperidin-3-yl)oxy)phenyl)piperidin-1-yl)acetate (699 mg, 1.39 mmol, Eq: 1) in EtOAc (6.95 ml) was added 4M HCl in dioxane (6.95 ml, 27.8 mmol, Eq: 20) and the RM stirred at RT overnight. The volatiles were removed by evaporation. The product has precipitated so it was filtered over glass fiber paper, washed with cold EtOA and dried under HV to gave the title compound (532.2 mg, 96%) as yellow solid.

### Ligase 36: 9-[(3S)-3-[[2-(2,6-dioxo-3-piperidyl)-1,3-dioxo-isoindolin-4-yl]amino]pyrrolidin-1-yl]-9-oxo-nonanoic acid

Ligase 36 was prepared in analogy to Ligase 11.

### Ligase 37: 2-(2,6-dioxo-3-piperidinyl)-5-(1-piperazinyl)-1H-Isoindole-1,3(2H)-dione (CAS: 2154342-61-5)

### Ligase 38: 3-(4-(piperidin-4-yl)phenoxy)piperidine-2,6-dione hydrochloride

### a) tert-butyl 4-[4-[(2,6-dioxo-3-piperidyl)oxy]phenyl]piperidine-1-carboxylate

To a solution of 1-BOC-4-p-hydroxyphenylpiperidine (5300 mg, 19.1 mmol, 1 eq) in DMF (150 mL) was added NaH (1910 mg, 47.7 mmol, 2.5 eq) then 3-bromopiperidine-2,6-dione (4769 mg, 24.8 mmol, 1.3 eq) was added, the mixture was stirred at 90° C. for 12 hours. It was filtered and concentrated. The residue was purified by prep-HPLC (FA) to get the title compound (4500 mg, 11.5 mmol, 60% yield) as a light yellow solid.

### b) 3-(4-(piperidin-4-yl)phenoxy)piperidine-2,6-dione hydrochloride

To a solution of tert-butyl 4-[4-[(2,6-dioxo-3-piperidyl)oxy]phenyl]piperidine-1-carboxylate (4000 mg, 10.3 mmol, 1 eq) in EtOAc (89 mL) was added HCl in EtOAc (2.57 mL, 10.3 mmol, 1 eq) the mixture was stirred at 25° C. for 1 h. It was filtered and the filter cake was purified by prep-HPLC (HCl) to get the title compound (1626 mg, 5.64 mmol, 47% yield) as a white solid.

### Ligase 39: 4-(4-(4-((2,6-dioxopiperidin-3-yl)oxy)phenyl)piperidin-1-yl)butanoic acid hydrochloride

### a) tert-butyl 4-(4-(4-((2,6-dioxopiperidin-3-yl)oxy)phenyl)piperidin-1-yl)butanoate

Ligase 38 (75 mg, 231 μmol, Eq: 1) was combined with DMF (770 μl) and tert-butyl 4-bromobutanoate (77.3 mg, 61.4 μl, 346 μmol, Eq: 1.5). DIPEA (119 mg, 161 μl, 924 μmol, Eq: 4) was added and the reaction mixture was stirred for 2 h. Potassium iodide (38.3 mg, 231 μmol, Eq: 1) was added and the reaction stirred for 16 hours. The reaction mixture was partionated between water and EtOAc. The aqueous layer was extracted with EtOAc. The organic layers were combined, washed with sat NaCl, dried over Na2SO4, filtered and concentrated in vacuo. The crude product was purified on silic gel (DCM/MeOH 0-15%) to yield the title compound as a light red solid (43 mg, 42%).

### b) 4-(4-(4-((2,6-dioxopiperidin-3-yl)oxy)phenyl)piperidin-1-yl)butanoic acid hydrochloride

tert-butyl 4-(4-(4-((2,6-dioxopiperidin-3-yl)oxy)phenyl)piperidin-1-yl)butanoate (40 mg, 92.9 μmol, Eq: 1) was dissolved in EtOAc (1.5 ml). 4N HCl in dioxane (465 μl, 1.86 mmol, Eq: 20) was added and the reaction mixture was stirred for 16 h. The reaction was concentrated in vacuo to yield the title compound (33.3 mg, 93%) as an off-white powder.

### Ligase 40: 3-(4-(4-((2,6-dioxopiperidin-3-yl)oxy)phenyl)piperidin-1-yl)propanoic acid

### a) tert-butyl 3-(4-(4-((2,6-dioxopiperidin-3-yl)oxy)phenyl)piperidin-1-yl)propanoate

Ligase 38 (151 mg, 465 μmol, Eq: 1) was suspended in DMF (1.55 ml). DIPEA (300 mg, 406 μl, 2.32 mmol, Eq: 5) and tert-butyl 3-bromopropanoate (117 mg, 93.1 μl, 558 μmol, Eq: 1.2) were added, and the reaction was stirred at 80° C. for 24 hours. Additional tert-butyl 3-bromopropanoate (19.4 mg, 15.5 μl, 93 μmol, Eq: 0.2) was added, and the reaction was stirred at 80 degree for 7 hours. Water was added and the precipitate collected by filtration and washed with water and a small amount of diethyl ether, then dried in vacuo. The oragnic and aqueous filtrates were separared, and the aqueous layer was extracted with EtOAc (3×20 mL). The combined ether and EtOAc layers were washed with brine (50 mL), dried (MgSO₄), filtered and concentrated in vacuo. 80 mg brown solid. The combined organic extracts were purified on silica gel (DCM/MeOH 0-7%) to provide the title compound as a light brown solid (131 mg).

### b) 3-(4-(4-((2,6-dioxopiperidin-3-yl)oxy)phenyl)piperidin-1-yl)propanoic acid

tert-butyl 3-(4-(4-((2,6-dioxopiperidin-3-yl)oxy)phenyl)piperidin-1-yl)propanoate (120 mg, 288 μmol, Eq: 1) was dissolved in DCM (1 ml) and trifluoroacetic acid (1.48 g, 1 mL, 13 mmol, Eq: 45.1) was added. The reaction was stirred at rt for 4 hours, the solvent evaporated and purified by prep HPLC to provide the title compound as a colourless solid (46 mg, 96% purity, 32% yield).

### Ligase 41: 3-[4-(1-piperazinyl)phenoxy]-2,6-piperidinedione (CAS: 2259852-17-8)

### Ligase 42: 2-[4-[4-[(2,6-dioxo-3-piperidyl)oxy]phenyl]piperazin-1-yl]acetic acid; 2,2,2-trifluoroacetic acid

### a) tert-butyl 2-(4-(4-((2,6-dioxopiperidin-3-yl)oxy)phenyl)piperazin-1-yl)acetate

A mixture of Ligase 41 (150 mg, 460 μmol, Eq: 1), tert-butyl 2-bromoacetate (180 mg, 136 μl, 921 μmol, Eq: 2) and DIPEA (357 mg, 482 μl, 2.76 mmol, Eq: 6) in DMF (2.5 ml) was stirred at rt for 2 h. The reaction mixture was evaporated directly on isolute. The crude material was purified on silica gel (DCM/MeOH 0-7%) to yield the title compound (138 mg, 342 μmol, 74% yield) as a light yellow oil.

### b) 2,2,2-trifluoroacetic acid compound with 2-(4-(4-((2,6-dioxopiperidin-3-yl)oxy)phenyl)piperazin-1-yl)acetic acid

tert-butyl 2-(4-(4-((2,6-dioxopiperidin-3-yl)oxy)phenyl)piperazin-1-yl)acetate (138 mg, 342 μmol, Eq: 1) was dissolved in DCM (3 ml) and TFA (780 mg, 527 μl, 6.84 mmol, Eq: 20) was added. The rm was stirred at rt overnight. The crude reaction mixture was concentrated in vacuo and dried on hv overnight to yield the title compound (215 mg, 340 μmol, 99% yield) as a light yellow oil.

### Ligase 43: 3-[[2-(2,6-dioxo-3-piperidyl)-1,3-dioxo-isoindolin-4-yl]amino]propanoic acid (CAS: 2225940-46-3)

### Ligase 44: 1-[2-(2,6-dioxo-3-piperidyl)-1,3-dioxo-isoindolin-5-yl]azetidine-3-carboxylic acid

To a stirred solution of 2-(2,6-dioxo-3-piperidyl)-5-fluoro-isoindoline-1,3-dione (30 mg, 109 μmol, 1.0 eq, CAS: 835616-61-0) in DMSO (0.5 mL) at room temperature was added azetidine-3-carboxylic acid (11 mg, 109 μmol, 1.0 eq) and DIPEA (28.1 mg, 37.9 μL, 217 μmol, 2.0 eq). The reaction mixture was stirred at 90° C. overnight and used directly in the next step.

### Ligase 45: 9-[4-[[2-(2,6-dioxo-3-piperidyl)-1,3-dioxo-isoindolin-4-yl]oxymethyl]triazol-1-yl]nonanal

### a) 2-(2,6-dioxopiperidin-3-yl)-4-(((1-(9-hydroxynonyl)-1H-1,2,3-triazol-4-yl)methyl)amino)isoindoline-1,3-dione

To a mixture of 2-(2,6-dioxo-3-piperidyl)-4-prop-2-ynoxy-isoindoline-1,3-dione (0.2 g, 640 umol) and 9-azidononan-1-ol (142 mg, 768 umol) in THF (4.0 mL) and water (1.0 mL) were added sodium ascorbate (253 mg, 1.28 mmol) followed by copper sulfate (204 mg, 1.28 mmol, 56 uL) at room temperature. The reaction mixture was stirred at room temperature for 3 h. Reaction mixture was added to water and extracted with 5% methanol in dichloromethane. The organic layer was washed with water, brine solution, dried over sodium sulfate, filtered and concentrated under reduced pressure. The crude residue was purified on silica (DCM/MeOH 0-10%) to afford the title compound (0.18 g, 320 umol, 50% yield) as an off-white solid. MS (ESI): 498.1 ([M+H]⁺).

### b) 9-[4-[[2-(2,6-dioxo-3-piperidyl)-1,3-dioxo-isoindolin-4-yl]oxymethyl]triazol-1-yl]nonanal

To a solution of 2-(2,6-dioxo-3-piperidyl)-4-[[1-(9-hydroxynonyl)triazol-4-yl]methoxy]isoindoline-1,3-dione (120 mg, 241 umol) in DCM (4.0 mL) was added Dess-Martin Periodinane (204 mg, 482 umol) at 0° C. under nitrogen atmosphere. The reaction mixture was warmed to room temperature and stirred for 2 h. Reaction mixture was filtered through celite bed and washed with DCM. The resulting filtrate was washed with water, brine solution, dried over sodium sulfate, filtered and concentrated under reduced pressure. The crude residue was purified on silica (DCM/MeOH 0-10%) to afford the title compound (55.0 mg, 96 umol, 39% yield) as an off-white solid. MS (ESI): 496.3 ([M+H]⁺).

### Ligase 46: 5-(2,6-diazaspiro[3.3]hept-2-yl)-2-(2,6-dioxo-3-piperidinyl)-1H-isoindole-1, 3(2H)-dione (CAS: 2226301-50-2)

### Ligase 47: 2-[[2-(2,6-dioxo-3-piperidinyl)-2,3-dihydro-1,3-dioxo-1H-isoindol-4-yl]oxy]acetic acid (CAS: 1061605-21-7)

### Ligase 48: 2-[2-(2,6-dioxo-3-piperidyl)-1,3-dioxo-isoindolin-5-yl]oxyacetic acid

### a) dimethyl 4-hydroxybenzene-1,2-dicarboxylate

To an ice-cooled solution of 4-hydroxyphthalic acid (20.0 g, 109 mmol, 1 eq) in methanol (500 mL) was added thionyl chloride (39 g, 329 mmol, 3 eq) dropwised over 5 min. The mixture was heated at 60° C. for 5 h. The reaction mixture was evaporated under high vacuum, the residue was taken up in EtOAc (300 mL) and concentrated to afford the title compound (19.5 g, 92 mmol, 84% yield) as a light yellow solid.

### b) dimethyl 4-(2-methoxy-2-oxo-ethoxy)benzene-1,2-dicarboxylate

A mixture of dimethyl 4-hydroxybenzene-1,2-dicarboxylate (19.0 g, 90.4 mmol, 1 eq), methyl bromoacetate (15.2 g, 99 mmol, 1.1 eq) and potassium carbonate (37 g, 271 mmol, 3 eq) in acetonitril (300 mL) was heated at 80° C. for 2 h. The reaction mixture was filtered, evaporated under high vacuum to afford the title compound (25 g, 88 mmol, 92% yield) as a yellow oil.

### c) 4-(carboxymethoxy)phthalic acid

To a solution of dimethyl 4-(2-methoxy-2-oxo-ethoxy)benzene-1,2-dicarboxylate (25.0 g, 88 mmol, 1 eq) in ethanol (300 mL) was added sodium hydroxide (300 mL, 900 mmol, 10.1 eq) in one portion. This mixture was heated at 80° C. for 12 h. The reaction mixture was concentrated and the remaining aqueous phase was acidified with HCl until PH≈1. The suspension was extracted with EtOAc/THF (1/1, 300 mL*3). The combined organics was washed with water and brine, dried over Na2SO4, filtered and filtration was evaporated to afford the title compound (12.4 g, 51 mmol, 58% yield) as a light yellow solid.

### d) 2-[2-(2,6-dioxo-3-piperidyl)-1,3-dioxo-isoindolin-5-yl]oxyacetic acid

A mixture of 4-(carboxymethoxy)phthalic acid (6.0 g, 24 mmol, 1 eq) and 3-aminopiperidine-2,6-dione hydrochloride (4.5 g, 27 mmol, 1.1 eq) in pyridine (120 mL) was stirred at 100° C. for 16 h. The mixture was concentrated and the residue was triturated in a mixed solvent of MeCN/EtOAc (1/1, 200 mL) and filtered, the filtration was concentrated. The residue was then triturated in a mixed solvent of MeOH/EtOAc (1/10, 30 mL) for 10 min. The suspension was filtered and the filter cake was washed with EtOAc and PE. The solid was collected and dried to afford the title compound (1500 mg, 4.51 mmol, 17% yield) as a light grey solid.

### Ligase 49: N-[2-(2,6-dioxo-3-piperidinyl)-2,3-dihydro-1,3-dioxo-1H-isoindol-4-yl]glycine (CAS: 927670-97-1)

### Ligase 50: 3-[[4-(4-piperidinyl)phenyl]amino]-2,6-piperidinedione (CAS: 2259851-37-9)

### Ligase 51: 2-(4-(4-((2,6-dioxopiperidin-3-yl)amino)phenyl)piperidin-1-yl)acetic acid

### a) tert-butyl 2-(4-(4-((2,6-dioxopiperidin-3-yl)amino)phenyl)piperidin-1-yl)acetate

A mixture of Ligase 50 hydrochloride (200 mg, 618 μmol, Eq: 1), tert-butyl 2-bromoacetate (157 mg, 119 μl, 803 μmol, Eq: 1.3) and N,N-diisopropylethylamine (399 mg, 539 μl, 3.09 mmol, Eq: 5) in DMF (4 ml) was stirred 2 hr at rt. The reaction mixture was poured into water and extracted with AcOEt (2×). The organic layers were combined, dried over Na2SO4 and concentrated in vacuo. The crude material was purified on silica gel (heptane/EtOAc 50-100%) to yield the title compound (164 mg, 66%) as a white solid.

### b) 2-(4-(4-((2,6-dioxopiperidin-3-yl)amino)phenyl)piperidin-1-yl)acetic acid

tert-butyl 2-(4-(4-((2,6-dioxopiperidin-3-yl)amino)phenyl)piperidin-1-yl)acetate (164 mg, 408 μmol, Eq: 1) was combined with DCM (3 ml) to give a colorless solution. 2,2,2-trifluoroacetic acid (1.48 g, 1 mL, 13 mmol, Eq: 32) was added at 0° C. then the reaction mixture was stirred at rt. The crude reaction mixture was concentrated in vacuo and dried to yield the title compound (264 mg, 141% yield) as a light blue solid.

### Ligase 52: 4-(4-(4-((2,6-dioxopiperidin-3-yl)amino)phenyl)piperidin-1-yl)butanoic acid hydrochloride

### a) benzyl 4-[4-[4-[(2,6-dioxo-3-piperidyl)amino]phenyl]-1-piperidyl]butanoate

A mixture of Ligase 50 (1100 mg, 3.4 mmol, 1 eq), benzyl 4-bromobutanoate (1746 mg, 6.8 mmol, 2 eq) and DIPEA (3.25 mL, 18.6 mmol, 5.5 eq) in DMF (30 mL) was stirred at 25° C. for 48 h. The reaction was extracted with EtOAc (120 mL×3). The combined organic layers were washed with brine (100 mL), dried over (Na2SO4), concentrated in vacuo. The residue was purified on silica gel (EtOAc:EtOH 2-10%) to afford the title compound (1.5 g, 3.2 mmol, 95% yield) as a grey solid.

### b) 4-(4-(4-((2,6-dioxopiperidin-3-yl)amino)phenyl)piperidin-1-yl)butanoic acid hydrochloride

A mixture of benzyl 4-[4-[4-[(2,6-dioxo-3-piperidyl)amino]phenyl]-1-piperidyl]butanoate (1.4 g, 3.0 mmol, 1 eq) and palladium on carbon (700 mg, 0.66 mmol, 0.22 eq) in 2-propanol (40 mL) was stirred at 60° C. for 20 h with hydrogen atmosphere (15.0 psi). The mixture was filtered, concentrated in vacuo to give the crude title compound (960 mg, 2.3 mmol, 68% yield) as a brown solid.

### Ligase 53: 3-(4-piperazin-1-ylanilino)piperidine-2,6-dione hydrochloride (CAS: 2259851-44-8)

### Ligase 54: 2-[4-[4-[(2,6-dioxo-3-piperidyl)amino]phenyl]piperazin-1-yl]acetic acid hydrochloride

### a) tert-butyl 2-[4-[4-[(2,6-dioxo-3-piperidyl)amino]phenyl]piperazin-1-yl]acetate

A mixture of Ligase 53 (1500 mg, 4.6 mmol, 1 eq), tert-butyl bromoacetate (1801 mg, 9.2 mmol, 2 eq) and DIPEA (2.41 mL, 13.8 mmol, 3 eq) in acetonitril (40 mL) was stirred at 25° C. for 15 h. The mixture was poured into water (150.0 mL) and extracted with EtOAc (120.0 mL×2). The combined organic layers were washed with brine (80.0 mL), dried over Na2SO4, and concentrated in vacuo. The residue was further purified by trituration in a mixture solvent (petroleum ether:ethyl acetate=1:1, 25 mL) to afford the title compound (1.25 g, 3.1 mmol, 67% yield) as a dark red solid.

### b) 2-[4-[4-[(2,6-dioxo-3-piperidyl)amino]phenyl]piperazin-1-yl]acetic acid hydrochloride

A mixture of tert-butyl 2-[4-[4-[(2,6-dioxo-3-piperidyl)amino]phenyl]piperazin-1-yl]acetate (1100 mg, 2.7 mmol, 1 eq) in HCl in EtOAc (25.0 mL, 100 mmol, 36 eq) was stirred at 25° C. for 12 h. The mixture was filtered and filter cake was washed with EtOAc (15.0 mL×3), dissolved in water (30 mL) and concentrated by lyophilization to give the title compound (1029 mg, 2.69 mmol, 92% yield) as a black solid.

### Ligase 55: 5-(4-amino-1-piperidyl)-2-(2,6-dioxo-3-piperidyl)isoindoline-1,3-dione

### a) tert-butyl N-[1-[2-(2,6-dioxo-3-piperidyl)-1,3-dioxo-isoindolin-5-yl]-4-piperidyl]carbamate

To a stirred solution of 2-(2,6-dioxo-3-piperidyl)-5-fluoro-isoindoline-1,3-dione (CAS 835616-61-0, 200 mg, 724 μmol, 1.0 eq) and tert-butyl piperidin-4-ylcarbamate (145 mg, 724 μmol, 1.0 eq) in DMSO (1.5 mL) at room temperature was added DIPEA (187 mg, 253 μL, 1.45 mmol, 2.0 eq). The reaction mixture was stirred at 90° C. overnight. The reaction mixture was poured into EtOAc/THF (1:1) and extracted sequentially with water and brine. The crude material was purified on silica column (DCM/MeOH 0-10%) to afford the title compound (315 mg, 690 μmol, 95% yield) as a yellow amorphous solid. MS (ESI): 457.4 ([M+H]⁺).

### b) 5-(4-amino-1-piperidyl)-2-(2,6-dioxo-3-piperidyl)isoindoline-1,3-dione

To a stirred solution of tert-butyl N-[1-[2-(2,6-dioxo-3-piperidyl)-1,3-dioxo-isoindolin-5-yl]-4-piperidyl]carbamate (315 mg, 690 μmol, 1.0 eq) in dioxane (6 mL) was added hexafluoropropan-2-ol (116 mg, 3.5 mL, 690 μmol, 1.0 eq) and the reaction mixture was heated at 130° C. for 15 min under microwave irradiation in a sealed microwave tube. The reaction mixture was concentrated in vacuo to afford the title compound (109 mg, 306 μmol, 44% yield) as a yellow solid. MS (ESI): 357.2 ([M+H]⁺).

### Ligase 56: 2-(2,6-dioxo-3-piperidyl)-5-[4-(methylamino)-1-piperidyl]isoindoline-1,3-dione

### a) tert-butyl N-[1-[2-(2,6-dioxo-3-piperidyl)-1,3-dioxo-isoindolin-5-yl]-4-piperidyl]-N-methyl-carbamate

To a stirred solution of 2-(2,6-dioxo-3-piperidyl)-5-fluoro-isoindoline-1,3-dione (200 mg, 724 μmol, 1.0 eq) and tert-butyl methyl(piperidin-4-yl)carbamate (155 mg, 724 μmol, 1.0 eq) in DMSO (1.5 mL) at room temperature was added DIPEA (187 mg, 253 μL, 1.45 mmol, 2.0 eq). The reaction mixture was stirred at 90° C. overnight. The reaction mixture was poured into EtOAc/THF (1:1) and extracted sequentially with water and brine. The crude material was purified on silica column (DCM/MeOH 0-10%) to afford the title compound (0.234 g, 472 μmol, 64% yield) as a yellow oil. MS (ESI): 471.3 ([M+H]⁺).

### b) 2-(2,6-dioxo-3-piperidyl)-5-[4-(methylamino)-1-piperidyl]isoindoline-1,3-dione

To a 5 ml microwave vial were added tert-butyl N-[1-[2-(2,6-dioxo-3-piperidyl)-1,3-dioxo-isoindolin-5-yl]-4-piperidyl]-N-methyl-carbamate (234 mg, 497 μmol, 1.0 eq) and hexafluoropropan-2-ol (83.6 mg, 2 mL, 497 μmol, 1.0 eq). The vial was capped and heated in the microwave at 130° C. for 3 h. The mixture was concentrated in vacuo to afford the title compound (110 mg, 267 μmol, 53% yield). MS (ESI): 371.2 ([M+H]⁺).

### Ligase 57: 9-[4-[3-[[2-(2,6-dioxo-3-piperidyl)-1,3-dioxo-isoindolin-4-yl]amino]propyl]triazol-1-yl]nonanal

### a) 2-(2,6-dioxopiperidin-3-yl)-4-(pent-4-yn-1-ylamino)isoindoline-1,3-dione

To a solution of 2-(2,6-dioxo-3-piperidyl)-4-fluoro-isoindoline-1,3-dione (0.5 g, 1.81 mmol) in DMSO (5 mL) was added pent-4-yn-1-amine hydrochloride (541 mg, 4.52 mmol) followed by DIPEA (701 mg, 5.43 mmol) at room temperature under nitrogen atmosphere. The reaction mixture was heated at 80° C. for 16 h. Reaction mixture was cooled to room temperature, added to water, resulting solid was filtered, washed with water and dried to afford title compound (0.18 g, 450 umol, 24% yield) as a light yellow solid. MS (ESI): 339.9 ([M+H]⁺).

### b) 2-(2,6-dioxopiperidin-3-yl)-4-((3-(1-(9-hydroxynonyl)-1H-1,2,3-triazol-4-yl)propyl)amino)isoindoline-1,3-dione

To a mixture of 2-(2,6-dioxo-3-piperidyl)-4-(pent-4-ynylamino)isoindoline-1,3-dione (0.18 g, 530 umol) and 9-azidononan-1-ol (98.2 mg, 530 umol) in THF (7.0 mL) and water (1.5 mL) were added copper sulfate (169 mg, 1.06 mmol) followed by sodium ascorbate (210 mg, 1.06 mmol) at room temperature. The reaction mixture was stirred at room temperature for 16 h. Reaction mixture was diluted with ethyl acetate and washed with water, brine solution, dried over sodium sulfate, filtered and concentrated under reduced pressure. The crude residue was purified on silica (DCM/MeOH 0-10%) to afford title compound (0.13 g, 235 umol, 44% yield) as a light yellow solid. MS (ESI): 525.0 ([M+H]⁺).

### c) 9-[4-[3-[[2-(2,6-dioxo-3-piperidyl)-1,3-dioxo-isoindolin-4-yl]amino]propyl]triazol-1-yl]nonanal

To a solution of 2-(2,6-dioxo-3-piperidyl)-4-[3-[1-(9-hydroxynonyl)triazol-4-yl]propylamino]isoindoline-1,3-dione (0.13 g, 247 umol) in DCM (5.0 mL) was added Dess-Martin Periodinane (157 mg, 371 umol) at 0° C. under nitrogen atmosphere. The reaction mixture was warmed to room temperature and stirred for 1 hour. Reaction mixture was filtered through celite bed and washed with dichloromethane. The resulting filtrate was washed with brine solution, dried over sodium sulfate, filtered and concentrated under reduced pressure. The crude residue was purified on silica (DCM/MeOH 0-10%) to afford title compound (60 mg, 62 umol, 25% yield) as a pale yellow semi solid. MS (ESI): 523.0 ([M+H]⁺).

### Ligase 58: 9-[4-[2-[[2-(2,6-dioxo-3-piperidyl)-1,3-dioxo-isoindolin-4-yl]amino]ethyl]triazol-1-yl]nonanal

### a) 4-(but-3-yn-1-ylamino)-2-(2,6-dioxopiperidin-3-yl)isoindoline-1,3-dione

To a solution of 2-(2,6-dioxo-3-piperidyl)-4-fluoro-isoindoline-1,3-dione (0.5 g, 1.81 mmol) in DMSO (5.0 mL) were added but-3-yn-1-amine (312 mg, 4.53 mmol) followed by DIPEA (701 mg, 5.43 mmol, 945 uL) at room temperature under nitrogen atmosphere. The raction mixture was heated at 80° C. for 16 h. Reaction mixture was cooloed to room temperature, diluted with water, resulting solid was filtered, washed with water and dried to afford tilte compound (0.19 g, 495 umol, 27% yield) as a light yellow soild. MS (ESI): 325.9 ([M+H]⁺).

### b) 2-(2,6-dioxopiperidin-3-yl)-4-((2-(1-(9-hydroxynonyl)-1H-1,2,3-triazol-4-yl)ethyl)amino)isoindoline-1,3-dione

To a mixture of 4-(but-3-ynylamino)-2-(2,6-dioxo-3-piperidyl)isoindoline-1,3-dione (0.19 g, 584 umol) and 9-azidononan-1-ol (108 mg, 584 umol) in THF (7.5 mL) and water (1.5 mL) were added copper sulfate (186 mg, 1.17 mmol) followed by sodium ascorbate (231 mg, 1.17 mmol) at room temperature. The reaction mixture was stirred at room temperature for 16 h. Reaction mixture was diluted water and extracted with EtOAc. The organic layer was washed with water, brine solution, dried over sodium sulfate, filtered and concentrated under reduced pressure. The crude residue was purified on silica (DCM/MeOH 0-10%) to afford the title compound (0.13 g, 239 umol, 40% yield) as a light yellow solid. MS (ESI): 511.0 ([M+H]⁺).

### c) 9-[4-[2-[[2-(2,6-dioxo-3-piperidyl)-1,3-dioxo-isoindolin-4-yl]amino]ethyl]triazol-1-yl]nonanal

To a solution of 2-(2,6-dioxo-3-piperidyl)-4-[2-[1-(9-hydroxynonyl)triazol-4-yl]ethylamino]isoindoline-1,3-dione (0.13 g, 254 umol) in DCM (5.0 mL) was added Dess-Martin Periodinane (161 mg, 381 umol) at 0° C. under nitrogen atmosphere. The reaction mixture was warmed to room temperature and stirred for 1 hour. Reaction mixture filtered through celite bed and wshed with dichloromethane. The resulting filtrate was washed with brine solution, dried over sodium sulfate, filtered and concentrated under reduced pressure. The crude residue was purified on silica (DCM/MeOH 0-10%) to afford the title compound (0.06 g, 68 umol, 26% yield) as a pale yellow semi solid. MS (ESI): 509.4 ([M+H]⁺).

### Ligase 59: 5-(3-aminoazetidin-1-yl)-2-(2,6-dioxo-3-piperidyl)isoindoline-1,3-dione

### a) tert-butyl N-[1-[2-(2,6-dioxo-3-piperidyl)-1,3-dioxo-isoindolin-5-yl]azetidin-3-yl]carbamate

To a stirred solution of 2-(2,6-dioxo-3-piperidyl)-5-fluoro-isoindoline-1,3-dione (200 mg, 724 μmol, 1.0 eq) and tert-butyl azetidin-3-ylcarbamate (125 mg, 724 μmol, 1.0 eq) in DMSO (1.5 mL) at room temperature was added DIPEA (187 mg, 253 μL, 1.45 mmol, 2.0 eq). The reaction mixture was stirred at 90° C. overnight. The reaction mixture was poured into EtOAc/THF (1:1) and extracted sequentially with water and brine. The crude material was purified on silica column (DCM/MeOH 0-10%) to afford the title compound (170 mg, 377 μmol, 52% yield) as a yellow solid. MS (ESI): 429.3 ([M+H]⁺).

### b) 5-(3-aminoazetidin-1-yl)-2-(2,6-dioxo-3-piperidyl)isoindoline-1,3-dione

To a 5 mL microwave vial were added tert-butyl N-[1-[2-(2,6-dioxo-3-piperidyl)-1,3-dioxo-isoindolin-5-yl]azetidin-3-yl]carbamate (170 mg, 397 μmol, 1.0 eq) and 1,1,1,3,3,3-hexafluoropropan-2-ol (66.7 mg, 2 mL, 397 μmol, 1.0 eq). The vial was capped and heated in the microwave at 135° C. for 25 min. The crude material was purified on silica column (DCM/MeOH 0-10%) to afford the title compound (72 mg, 219 μmol, 55% yield) as a yellow solid. MS (ESI): 329.2 ([M+H])+.

### Ligase 60: 2-(2,6-dioxo-3-piperidyl)-5-[3-(methylamino)azetidin-1-yl]isoindoline-1,3-dione

### a) tert-butyl N-[1-[2-(2,6-dioxo-3-piperidyl)-1,3-dioxo-isoindolin-5-yl]azetidin-3-yl]-N-methyl-carbamate

To a stirred solution of 2-(2,6-dioxo-3-piperidyl)-5-fluoro-isoindoline-1,3-dione (CAS 835616-61-0, 200 mg, 724 μmol, 1.0 eq) and tert-butyl azetidin-3-yl(methyl)carbamate (CAS 577777-20-9, 135 mg, 724 μmol, 1.0 eq) in DMSO (1.5 mL) at room temperature was added DIPEA (187 mg, 253 μL, 1.45 mmol, 2.0 eq). The reaction mixture was stirred at 90° C. overnight. The reaction mixture was cooled to room temperature and poured into EtOAc/THF (1:1) and then extracted sequentially with water and with brine. The crude material was purified on silica column (DCM/MeOH 0-10%) to afford the title compound (420 mg, 712 μmol, 98% yield) as a yellow solid. MS (ESI): 443.3 ([M+H]⁺).

### b) 2-(2,6-dioxo-3-piperidyl)-5-[3-(methylamino)azetidin-1-yl]isoindoline-1,3-dione

To a 5 ml microwave vial were added tert-butyl N-[1-[2-(2,6-dioxo-3-piperidyl)-1,3-dioxo-isoindolin-5-yl]azetidin-3-yl]-N-methyl-carbamate (170 mg, 384 μmol, 1.0 eq) and hexafluoropropan-2-ol (64.6 mg, 2 ml, 384 μmol, 1.0 eq). The vial was capped and heated in the microwave at 135° C. for 30 min. The vial was capped again and heated in the microwave at 135° C. for 2 h. The crude material was purified on silica column (DCM/MeOH 0-10%) to afford the title compound (170 mg, 382 μmol, 100% yield) as a yellow solid. MS (ESI): 343.2 ([M+H]⁺).

### Ligase 61: 2-(2,6-dioxo-3-piperidyl)-4-[[1-(4-piperidylmethyl)triazol-4-yl]methoxy]isoindoline-1,3-dione

### a) tert-butyl 4-((4-(((2-(2,6-dioxopiperidin-3-yl)-1,3-dioxoisoindolin-4-yl)oxy)methyl)-1H-1,2,3-triazol-1-yl)methyl)piperidine-1-carboxylate

To a mixture of 2-(2,6-dioxo-3-piperidyl)-4-prop-2-ynoxy-isoindoline-1,3-dione (500 mg, 1.6 mmol) and tert-butyl 4-(azidomethyl)piperidine-1-carboxylate (384 mg, 1.6 mmol) in THF (10 mL) was added a mixture of sodium ascorbate (634 mg, 3.2 mmol) and copper sulfate (857 mg, 3.2 mmol) in water (1.5 mL) at room temperature. The reaction mixture was stirred at room temperature for 12 h. Reaction mixture was diluted with water and extracted with ethyl acetate. The organic layer was washed with water, brine solution, dried over sodium sulfate, filtered and concentrated under reduced pressure. The crude residue was purified on silica column (DCM/MeOH 0-10%) to afford the title compound (625 mg, 1.11 mmol, 69% yield) as an off-white solid. MS (ESI): 551.3 ([M−H]⁻).

### b) 2-(2,6-dioxo-3-piperidyl)-4-[[1-(4-piperidylmethyl)triazol-4-yl]methoxy]isoindoline-1,3-dione

To a solution of tert-butyl 4-[[4-[[2-(2,6-dioxo-3-piperidyl)-1,3-dioxo-isoindolin-4-yl]oxymethyl]triazol-1-yl]methyl]piperidine-1-carboxylate (625 mg, 1.13 mmol) in DCM (6.0 mL) was added trifluoroacetic acid (644 mg, 5.66 mmol, 435 uL) at 0° C. under nitrogen atmosphere. The reaction mixture was warmed to room temperature and stirred for 1 hour. Reaction mixture was concentrated under reduced pressure and dried to afford title compound (750 mg, 1.06 mmol, 93% yield) as a light brown oil (TFA salt).

### Ligase 62: 9-[6-[[[2-(2,6-dioxo-3-piperidyl)-1,3-dioxo-isoindolin-4-yl]amino]methyl]-3-pyridyl]nonanal

### a) benzyl ((5-(9-((tert-butyldimethylsilyl)oxy)non-1-yn-1-yl)pyridin-2-yl)methyl)carbamate

A solution of benzyl N-[(5-bromo-2-pyridyl)methyl]carbamate (1.2 g, 3.73 mmol) in piperidine (15.0 mL) was purged with nitrogen gas for 20 minutes followed by the addition of tetrakis(triphenylphosphine)palladium(0) (570 mg, 373 umol) and copper (I) iodide (71 mg, 373 umol) at room temperature. The reaction mixture was heated at 50° C. for 20 minutes and added tert-butyl-dimethyl-non-8-ynoxy-silane (0.95 g, 3.73 mmol). The reaction mixture was heated at 80° C. for 2 h. Reaction mixture was cooled to room temperature, filtered through celite pad and washed with ethyl acetate. The resulitng filtrate was washed with water, brine solution, dried over sodium sulfate, filtered and concentrated under reduced pressure. The crude residue was purified on silica (PE/EtOAc 0-100%) to afford the title compound (1.0 g, 1.79 mmol, 47% yield) as a light brown semi solid. MS (ESI): 495.3 ([M+H]⁺).

### b) benzyl ((5-(9-((tert-butyldimethylsilyl)oxy)nonyl)pyridin-2-yl)methyl)carbamate

A mixture of benzyl N-[[5-[9-[tert-butyl(dimethyl)silyl]oxynon-1-ynyl]-2-pyridyl]methyl]carbamate (1.0 g, 2.02 mmol) and wet 10% Palladium on carbon (860 mg, 8.09 mmol) in methanol (20 mL) was stirred at room temperature under the pressure of hydrogen gas (1 atm) for 16 h. The reaction mixture was filtered through celite pad and washed with methanol and concentrated under reduced pressure to afford title compound (500 mg, 374 umol, 18% yield) as a light brown solid. MS (ESI): 365.5 ([M+H]⁺).

### c) 4-(((5-(9-((tert-butyldimethylsilyl)oxy)nonyl)pyridin-2-yl)methyl)amino)-2-(2,6-dioxopiperidin-3-yl)isoindoline-1,3-dione

To a mixture of 2-(2,6-dioxo-3-piperidyl)-4-fluoro-isoindoline-1,3-dione (153 mg, 556 umol) and [5-[9-[tert-butyl(dimethyl)silyl]oxynonyl]-2-pyridyl]methanamine (506 mg, 1.39 mmol) in DMSO (5.0 mL) was added DIPEA (215 mg, 1.67 mmol, 290 uL) at room temperature under nitrogen atmosphere. The reaction mixture was heated at 80° c. for 16 h. Reaction mixture was cooled to room temperature, added to water and extracted with ethyl acetate. The organic layer was washed with water, brine solution, dried over sodium sulfate, filtered and concentrated under reduced pressure. The crude residue was purified on silica (DCM/MeOH 0-10%) to afford the title compound (0.24 g, 141 umol, 25% yield) as an off-white semi solid. MS (ESI): 621.3 ([M+H]⁺).

### d) 2-(2,6-dioxopiperidin-3-yl)-4-(((5-(9-hydroxynonyl)pyridin-2-yl)methyl)amino)isoindoline-1,3-

To a solution of 4-[[5-[9-[tert-butyl(dimethyl)silyl]oxynonyl]-2-pyridyl]methylamino]-2-(2,6-dioxo-3-piperidyl)isoindoline-1,3-dione (150 mg, 241 umol) in methanol (2.0 mL) was added p-toluenesulfonic acid (41.6 mg, 241 umol) at room temperature. The reaction mixture was stirred at room temperature for 3 h. Reaction mixture was concentrated under reduced pressure and the crude residue was purified on silica (DCM/MeOH 0-10%) to afford the title compound (80.0 mg, 112 umol, 46% yield) as an off-white solid. MS (ESI): 507.3 ([M+H]⁺).

### e) Ligase 62: 9-[6-[[[2-(2,6-dioxo-3-piperidyl)-1,3-dioxo-isoindolin-4-yl]amino]methyl]-3-pyridyl]nonanal

To a solution of 2-(2,6-dioxo-3-piperidyl)-4-[[5-(9-hydroxynonyl)-2-pyridyl]methylamino]isoindoline-1,3-dione (70 mg, 138 umol) in dichloromethane (2.5 mL) was added Dess-Martin Periodinane (117 mg, 276 umol) at 0° C. under nitrogen atmosphere. The reaction mixture was warmed to room temperature and stirred for 2 h. Reaction mixture was filtered through celite bed and washed with dichloromethane. The resulting filtrate was washed with water, brine solution, dried over sodium sulfate, filtered and concentrated under reduced pressure. The crude residue was purified on silica (DCM/MeOH 0-10%) to afford the title compound (25.0 mg, 25 umol, 18% yield) as an off-white semi solid. MS (ESI): 505.4 ([M+H]⁺).

### Ligase 63: 5-[4-[[2-(2,6-dioxo-3-piperidyl)-1,3-dioxo-isoindolin-4-yl]oxymethyl]triazol-1-yl]pentanoic acid

### a) tert-butyl 5-(4-(((2-(2,6-dioxopiperidin-3-yl)-1,3-dioxoisoindolin-4-yl)oxy)methyl)-1H-1,2,3-triazol-1-yl)pentanoate

To a mixture of 2-(2,6-dioxo-3-piperidyl)-4-prop-2-ynoxy-isoindoline-1,3-dione (100 mg, 320 umol) and tert-butyl 5-azidopentanoate (127 mg, 640 umol) in THF (5.0 mL) and water (2.5 mL) was added sodium ascorbate (126 mg, 640 umol) and copper sulfate (171 mg, 640 umol) at room temperature. The reaction mixture was stirred at room temperature for 12 h. Reaction mixture was diluted with water extracted with EtOAc. The organic layer was washed with water, brine solution, dried over sodium sulfate, filtered and concentrated under reduced pressure. The crude residue was purified on silica (PE/EtOAc 0-80%) to afford the title compound (35.0 mg, 58 umol, 18% yield) as an off-white solid. MS (ESI): 512.2 ([M+H]⁺).

### b) 5-[4-[[2-(2,6-dioxo-3-piperidyl)-1,3-dioxo-isoindolin-4-yl]oxymethyl]triazol-1-yl]pentanoic acid

To a solution of tert-butyl 5-[4-[[2-(2,6-dioxo-3-piperidyl)-1,3-dioxo-isoindolin-4-yl]oxymethyl]triazol-1-yl]pentanoate (35.0 mg, 68.42 umol) in dichloromethane (1.0 mL) was added trifluoroacetic acid (78.0 mg, 684 umol, 52 uL) at 0° C. under nitrogen atmosphere. The reaction mixture was warmed to room temperature and stirred for 1 hour.

Reaction mixture was concentrated under reduced pressure and dried to afford title compound (35.0 mg, crude) as a brown semi solid (TFA salt). MS (ESI): 456.2 ([M+H]⁺).

### Ligase 64: 2-(2,6-dioxo-3-piperidyl)-4-[[1-(4-piperidyl)triazol-4-yl]methoxy]isoindoline-1,3-dione

### a) 2-(2,6-dioxopiperidin-3-yl)-4-(prop-2-yn-1-yloxy)isoindoline-1,3-dione

To a solution of 2-(2,6-dioxo-3-piperidyl)-4-hydroxy-isoindoline-1,3-dione (5.0 g, 18.2 mmol) in DMF (50 mL) were added sodium carbonate (1.93 g, 18.2 mmol) followed by 3-bromoprop-1-yne (2.17 g, 18.2 mmol) at room temperature under nitrogen atmosphere. The reaction mixture was heated at 60° C. for 24 h. Reaction mixture was cooled to room temperature, added to water and extracted with ethyl acetate. The organic layer was washed with water, brine solution, dried over sodium sulfate, filtered and concentrated under reduced pressure. The crude residue was purified on silica (DCM/MeOH 0-10%) to afford the title compound (4.40 g, 14.0 mmol, 77% yield) as an off-white solid, MS (ESI): 313.1 ([M+H]⁺).

### b) tert-butyl 4-(4-(((2-(2,6-dioxopiperidin-3-yl)-1,3-dioxoisoindolin-4-yl)oxy)methyl)-1H-1,2,3-triazol-1-yl)piperidine-1-carboxylate

To a mixture of 2-(2,6-dioxo-3-piperidyl)-4-prop-2-ynoxy-isoindoline-1,3-dione (0.1 g, 320 umol) and tert-butyl 4-azidopiperidine-1-carboxylate (144 mg, 640 umol) in tetrahydrofuran (5.0 mL) and water (2.5 mL) were added sodium ascorbate (126 mg, 640 umol) and copper sulfate (102 mg, 640 umol, 28.4 uL) at room temperature. The reaction mixture was stirred at room temperature for 12 h. Reaction mixture was diluted with water and extracted with ethyl acetate. The organic layer was washed with water, brine solution, dried over sodium sulfate, filtered and concentrated under reduced pressure. The crude residue was purified on silica (PE/EtOAc 0-80%) to afford the title compound (35.0 mg, 57.3 umol, 17% yield) as an off-white solid. MS (ESI): 537.0 ([M−H]⁻).

### c) 2-(2,6-dioxo-3-piperidyl)-4-[[1-(4-piperidyl)triazol-4-yl]methoxy]isoindoline-1,3-dione

To a solution of tert-butyl 4-[4-[[2-(2,6-dioxo-3-piperidyl)-1,3-dioxo-isoindolin-4-yl]oxymethyl]triazol-1-yl]piperidine-1-carboxylate (35.0 mg, 64 umol) in DCM (1.0 mL) was added trifluoroacetic acid (74.1 mg, 649 umol, 50 uL) at 0° C. under nitrogen atmosphere. The reaction mixture was warmed to room temperature and stirred for 1 hour. Reaction mixture was concentrated under reduced pressure and dried to afford title compound (35.0 mg, crude) as a brown semi solid (TFA salt). MS (ESI): 439.0 ([M+H]⁺).

### Ligase 65: 9-[2-[[[2-(2,6-dioxo-3-piperidyl)-1,3-dioxo-isoindolin-4-yl]amino]methyl]-4-pyridyl]nonanal

### a) benzyl ((4-(9-((tert-butyldimethylsilyl)oxy)non-1-yn-1-yl)pyridin-2-yl)methyl)carbamate

A solution of benzyl N-[(4-bromo-2-pyridyl)methyl]carbamate (1.30 g, 4.05 mmol) in piperidine (15.0 mL) was purged with nitrogen gas for 20 minutes, followed by the addition of tetrakis(triphenylphosphine)palladium(0) (618 mg, 404 umol) and copper (I) iodide (77 mg, 404 umol, 13.7 uL) at room temperature. The reaction mixture was heated at 50° c. for 20 minutes and was added tert-butyl-dimethyl-non-8-ynoxy-silane (1.03 g, 4.05 mmol). Reaction mixture was heated at 80° c. for 2 h. The reaction mixture was cooled to room temperature, filtered through celite pad and washed with ethyl acetate. The resulting filtrate was washed with water, brine solution, dried over sodium sulfate, filtered and concentrated under reduced pressure. The crude residue was purified on silica (PE/EtOAc 0-100%) to afford the title compound (1.40 g, 2.38 mmol, 58% yield) as a light brown semi solid. MS (ESI): 495.4 ([M+H]⁺).

### b) (4-(9-((tert-butyldimethylsilyl)oxy)nonyl)pyridin-2-yl)methanamine

A mixture of benzyl N-[[4-[9-[tert-butyl(dimethyl)silyl]oxynon-1-ynyl]-2-pyridyl]methyl]carbamate (1.40 g, 2.83 mmol) and wet 10% Palladium on carbon (602 mg, 5.66 mmol) in methanol (20 mL) was stirred at room temperature under the pressure of hydrogen gas (1 atm) for 16 h. The reaction mixture was filtered through celite pad and washed with methanol and concentrated under reduced pressure to afford title compound (0.72 g, 631 umol, 22% yield) as a brown solid. MS (ESI): 365.4 ([M+H]⁺).

### c) 4-(((4-(9-((tert-butyldimethylsilyl)oxy)nonyl)pyridin-2-yl)methyl)amino)-2-(2,6-dioxopiperidin-3-yl)isoindoline-1,3-dione

To a mixture of 2-(2,6-dioxo-3-piperidyl)-4-fluoro-isoindoline-1,3-dione (217.6 mg, 788 umol) and [4-[9-[tert-butyl(dimethyl)silyl]oxynonyl]-2-pyridyl]methanamine (718 ug, 1.97 umol) in DMSO (8.0 mL) was added DIPEA (101.8 mg, 788 umol, 137 uL) at room temperature under nitrogen atmosphere. The reaction mixture was heated at 80° C. for 16 h.

Reaction mixture was cooled to room temperature, added to water and extracted with ethyl acetate. The organic layer was washed with water, brine solution, dried over sodium sulfate, filtered and concentrated under reduced pressure. The crude residue was purified on silica (DCM/MeOH 0-10%) to afford title compound (0.4 g, 244 umol, 30% yield) as an off-white semi solid. MS (ESI): 621.3 ([M+H]⁺).

### d) 2-(2,6-dioxopiperidin-3-yl)-4-(((4-(9-hydroxynonyl)pyridin-2-yl)methyl)amino)isoindoline-1,3-dione

To a solution of 4-[[4-[9-[tert-butyl(dimethyl)silyl]oxynonyl]-2-pyridyl]methylamino]-2-(2,6-dioxo-3-piperidyl)isoindoline-1,3-dione (130 mg, 209 umol) in methanol (4.0 mL) was added p-toluenesulfonic acid (36.0 mg, 209 umol) at room temperature. The reaction mixture was stirred at room temperature for 3 h. Reaction mixture was concentrated under reduced pressure and the crude residue was purified on silica (DCM/MeOH 0-10%) to afford the title compound (80.0 mg, 98 umol, 46% yield) as an off-white semi solid. MS (ESI): 507.3 ([M+H]⁺).

### e) 9-(2-(((2-(2,6-dioxopiperidin-3-yl)-1,3-dioxoisoindolin-4-yl)amino)methyl)pyridin-4-yl)nonanal

To a solution of 2-(2,6-dioxo-3-piperidyl)-4-[[4-(9-hydroxynonyl)-2-pyridyl]methylamino]isoindoline-1,3-dione (80.0 mg, 157 umol) in DCM (2.0 mL) was added Dess-Martin Periodinane (100 mg, 236 umol) at 0° C. under nitrogen atmosphere. The reaction mixture was warmed to room temperature and stirred for 2 h. The reaction mixture was filtered through celite bed and washed with dichloromethane. The resulting filtrate was washed with water, brine solution, dried over sodium sulfate, filtered and concentrated under reduced pressure. The crude residue was purified on silica (DCM/MeOH 0-10%) to afford the title compound (30.0 mg, 25 umol, 16% yield) as an off-white solid. MS (ESI): 505.2 ([M+H]⁺).

### Ligase 66: 5-[4-(3-bromopropyl)-1-piperidyl]-2-(2,6-dioxo-3-piperidyl)isoindoline-1,3-dione

### a) 2-(2,6-dioxo-3-piperidyl)-4-[4-(3-hydroxypropyl)-1-piperidyl]isoindoline-1,3-dione

To the mixture of 2-(2,6-dioxo-3-piperidyl)-5-fluoro-isoindoline-1,3-dione (3.0 g, 10.8 mmol, 1 eq) in DMSO (30 mL) was added 3-(4-piperidyl)propan-1-ol (1.87 g, 13.0 mmol, 1.2 eq), DIPEA (5.68 mL, 32 mmol, 3 eq). The mixture was stirred at 100° C. for 12 h.

Water was added and extracted with EtOAc (200 mL*4). The organic phase was concentrated in vacuo. The residue was purified on silica gel (PE:EtOAc 10-50%) to give the title compound (2 g, 5 mmol, 46% yield) as yellow oil.

### b) 5-[4-(3-bromopropyl)-1-piperidyl]-2-(2,6-dioxo-3-piperidyl)isoindoline-1,3-dione

To the mixture of 2-(2,6-dioxo-3-piperidyl)-4-[4-(3-hydroxypropyl)-1-piperidyl]isoindoline-1,3-dione (2000 mg, 5 mmol, 1 eq) in THF (1 mL) was added carbon tetrabromide (3320 mg, 10 mmol, 2 eq), triphenylphosphine (2626 mg, 10 mmol, 2 eq). The mixture was stirred at 25° C. for 12 h. The mixture was concentrated in vacuo. The crude product was purified by prep-HPLC (neutral) to give the title compound (1532 mg, 3.3 mmol, 60% yield) as a yellow solid.

### Ligase 67: 2-(2,6-dioxo-3-piperidyl)-5-[rel-(3aS,6aR)-2,3,3a,4,6,6a-hexahydro-1H-pyrrolo[3,4-c]pyrrol-5-yl]isoindoline-1,3-dione hydrochloride (CAS 2229723-90-2)

### Ligase 68: 2-(2,6-dioxopiperidin-3-yl)-5-(piperazin-1-ylmethyl)isoindoline-1,3-dione hydrochloride

### a) dimethyl 4-(bromomethyl)benzene-1,2-dicarboxylate

To a solution of dimethyl 4-methylbenzene-1,2-dicarboxylate (6.0 g, 28.8 mmol, 1 eq) and N-bromosuccinimide (5.39 g, 30.2 mmol, 1.05 eq) in acetonitrile (50 mL) was added 2,2′-azobis(2-methylpropionitrile) (0.95 g, 5.7 mmol, 0.2 eq), then the mixture was stirred at 80° C. for 12 h under nitrogen. The reaction mixture was concentrated under vacuum. The residue was purified on silica gel (PE/EtOAc 20-50%) to provide the title compound (8 g, 27.8 mmol, 82% yield) as a yellow oil.

### b) dimethyl 4-[(4-tert-butoxycarbonylpiperazin-1-yl)methyl]benzene-1,2-dicarboxylate

To a solution of 1-BOC-piperazine (5.71 g, 30.65 mmol, 1.1 eq) in DMSO (50 mL) was added DIPEA (14.5 mL, 83.5 mmol, 3 eq), followed by dimethyl 4-(bromomethyl)benzene-1,2-dicarboxylate (8.0 g, 27.8 mmol, 1 eq). The mixture was stirred at 90° C. for 12 h. Ethyl acetate (300 mL) was added. The reaction was washed with water (50 mL*3). The organic phase was concentrated under vacuum. The residue was purified on silica gel (PE/EtOAc 10-50%) to provide the title compound (8.8 g, 22.4 mmol, 45% yield) as yellow oil.

### c) 4-[(4-tert-butoxycarbonylpiperazin-1-yl)methyl]phthalic acid

To a solution of dimethyl 4-[(4-tert-butoxycarbonylpiperazin-1-yl)methyl]benzene-1,2-dicarboxylate (8.8 g, 22.4 mmol, 1 eq) in THF (65 mL) was added sodium hydroxide (8.97 g, 224 mmol, 10 eq) and water (15 mL). The mixture was stirred at 50° C. for 12 h. Water (200 mL) was added, then extracted with EtOAc (150 mL*2). The aqueous phase was concentrated under vacuum to yield the title compound (8 g, 21.9 mmol, 91% yield) as white solid.

### d) tert-butyl 4-[[2-(2,6-dioxo-3-piperidyl)-1,3-dioxo-isoindolin-5-yl]methyl]piperazine-1-carboxylate

To solution of 4-[(4-tert-butoxycarbonylpiperazin-1-yl)methyl]phthalic acid hydrochloride (1.5 g, 4.12 mmol, 1 eq) in pyridine (15 mL) was added 3-aminopiperidine-2,6-dione (0.68 g, 4.12 mmol, 1 eq). The mixture was stirred at 80° C. for 20 h. Water (150 mL) was added, then extracted with EtOAc (100 mL*3). Combined extracts were washed with brine (50 mL), dried over sodium sulfate, filtered, and concentrated under vacuum. The residue was purified by pre-HPLC to provide the title compound (500 mg, 1.1 mmol, 25% yield) as grey solid.

### e) 2-(2,6-dioxo-3-piperidyl)-5-(piperazin-1-ylmethyl)isoindoline-1,3-dione hydrochloride

To a solution of tert-butyl 4-[[2-(2,6-dioxo-3-piperidyl)-1,3-dioxo-isoindolin-5-yl]methyl]piperazine-1-carboxylate (450 mg, 0.99 mmol, 1 eq) in DCM (20 mL) was added 4N HCl in EtOAc (25 mL, 100 mmol, 101 eq) at 0° C. The mixture was stirred at 0° C. for 2 h. The reaction was concentrated under vacuum. The residue was added water (20 mL) and lyophilized to yield the title compound (400 mg, 1.02 mmol, 101% yield) as light brown solid.

### Ligase 69: 3-[4-(4-piperidyl)phenyl]piperidine-2,6-dione hydrochloride

### a) tert-butyl 4-(4-bromophenyl)-3,6-dihydro-2H-pyridine-1-carboxylate

A mixture of 1-N—BOC-4-(4,4,5,5-tetramethyl-[1,3,2]dioxaborolan-2-yl)-3,6-dihydro-2H-pyridine (18 g, 58.3 mmol, 1.1 eq), 1-bromo-4-iodobenzene (15 g, 53 mmol, 1 eq), tetrakis(triphenylphosphine)palladium(0) (3.06 g, 2.65 mmol, 0.05 eq) and phosphoric acid, potassium salt (13 g, 159 mmol, 3 eq) in 1,4-dioxane (150 mL) and water (50 mL) was stirred at 95° C. for 10 h under N2 atmosphere. It was filtered and concentrated, the residue was purified by prep-HPLC (FA) to give the title compound (14 g, 41.3 mmol, 78% yield) as yellow oil.

### b) tert-butyl 4-[4-(4,4,5,5-tetramethyl-1,3,2-dioxaborolan-2-yl)phenyl]-3,6-dihydro-2H-pyridine-1-carboxylate

To a solution of tert-butyl 4-(4-bromophenyl)-3,6-dihydro-2H-pyridine-1-carboxylate (14.0 g, 41.3 mmol, 1 eq) bis(pinacolato)diboron (12612 mg, 49.6 mmol, 1.2 eq) KOAc (12186 mg, 124.1 mmol, 3 eq) in 1,4-dioxane (304 mL) was added Pd(dppf)Cl2.CH2Cl2 (3377 mg, 4.14 mmol, 0.1 eq), the mixture was purged with N2 for 3 times and stirred at 100° C. for 16 hours under N2 atomosphere. It was filtered and concentrated. The residue was purified on silica gel (PE:EtOAc 1-5%) to give the title compound (8500 mg, 22 mmol, 53% yield) as a white solid.

### c) 2,6-dibenzyloxy-3-bromo-pyridine

To a solution of 2,6-dibenzyloxypyridine (20 g, 68.6 mmol, 1 eq) in MeCN (300 mL) was added N-bromosuccinimide (9.7 g, 54.9 mmol, 0.8 eq) the mixture was stirred at 90° C. for 16 hours. It was concentrated. The residue was purified by chromatography on silica gel (PE:EtOAc 1-10%) to give the title compound (19 g, 51 mmol, 74% yield) as a white solid.

### d) tert-butyl 4-[4-(2,6-dibenzyloxy-3-pyridyl)phenyl]-3,6-dihydro-2H-pyridine-1-carboxylate

To a solution of tert-butyl 4-[4-(4,4,5,5-tetramethyl-1,3,2-dioxaborolan-2-yl)phenyl]-3,6-dihydro-2H-pyridine-1-carboxylate (5000 mg, 12.9 mmol, 1 eq) 2,6-dibenzyloxy-3-bromo-pyridine (3843 mg, 10.3 mmol, 0.8 eq), Pd(dppf)Cl2.CH2Cl2 (10582 mg, 1.3 mmol, 0.1 eq), Na2CO3 (3438 mg, 32.4 mmol, 2.5 eq) in DMF (100 mL) and water (20 mL) was purged with N2, and the mixture was stirred at 100° C. for 12 hours. It was filtered and concentrated. The residue was purified by prep-HPLC (FA) to get the title compound (4050 mg, 7.38 mmol, 56% yield) as a yellow oil.

### e) tert-butyl 4-[4-(2,6-dioxo-3-piperidyl)phenyl]piperidine-1-carboxylate

To a solution of tert-butyl 4-[4-(2,6-dibenzyloxy-3-pyridyl)phenyl]-3,6-dihydro-2H-pyridine-1-carboxylate (4000 mg, 7.29 mmol, 1 eq) in ethanol (120 mL) was added Pd/C (mg, 0.73 mmol, 0.1 eq), PtO2 (mg, 0.360 mmol, 0.050 eq) and AcOH (0.5 mL, 7.29 mmol, 1 eq) the mixture was purged with H2 for 3 times, and stirred at 30° C. for 3 hours under H2 atomosphere. It was filtered and concentrated. The residue was purified with prep-HPLC (FA) to get the title compound (1510 mg, 4.0 mmol, 54% yield) as a white solid.

### f) 3-[4-(4-piperidyl)phenyl]piperidine-2,6-dione hydrochloride

To a solution of tert-butyl 4-[4-(2,6-dioxo-3-piperidyl)phenyl]piperidine-1-carboxylate (1000 mg, 2.68 mmol, 1 eq) in EtOAc (10 mL) was added 4M HCl in dioxane (10 mL, 40 mmol, 14.9 eq). The mixture was stirred at 25 C for 2 hours. It was filtered and the filter cake was washed with EtOAc (5 mL*1), the filter cake was dissolved in water (40 mL) and lyophilized to get the title compound (758 mg, 2.46 mmol, 91% yield) as a white solid.

### Ligase 70: 3-(4-piperazin-1-ylphenyl)piperidine-2,6-dione;hydrochloride

### a) tert-butyl 4-(4-(2,6-bis(benzyloxy)pyridin-3-yl)phenyl)piperazine-1-carboxylate

To a stirred solution of tert-butyl 4-[4-(4,4,5,5-tetramethyl-1,3,2-dioxaborolan-2-yl)phenyl]piperazine-1-carboxylate (2 g, 5.15 mmol) dissolved in DMF (4 mL) and water (0.5 mL), sodium carbonate (1.09 g, 10.3 mmol) was added. The resulting solution was degassed with nitrogen gas for 15 minutes. [1,1′-Bis(diphenylphosphino)ferrocene]dichloropalladium(II) complex with dichloromethane (420 mg, 515 umol) was then added and the mixture was heated to 100° C. in a sealed tube. After 5 h, the reaction was judged complete and the mixture was filtered through celite. The filtrate was diluted with ice water and the product was extracted with ethyl acetate (3×50 mL). The combined organic layers were washed with brine, dried over sodium sulfate and concentrated under vacuum, The crude residue was purified by column chromatography to afford the title compound (1.4 g, 2.28 mmol, 44% yield) as a yellow solid. MS (ESI): 552.5 ([M+H]⁺).

### b) tert-butyl 4-(4-(2,6-dioxopiperidin-3-yl)phenyl)piperazine-1-carboxylate

A stirred solution of tert-butyl 4-[4-(2,6-dibenzyloxy-3-pyridyl)phenyl]piperazine-1-carboxylate (22 g, 39.8 mmol) dissolved in ethyl acetate (120 mL) and ethanol (120 mL) was degassed with argon for 20 minutes. Palladium on carbon (8.49 g, 7.98 mmol) was added to the reaction mixture. It was stirred at room temperature for 16 h with hydrogen gas bubbling through the solution. After 16 h, the reaction mixture was filtered through celite, concentrated under reduced pressure and purified by column chromatography eluting (DCM/MeOH 98:2) to afford the title compound (13.2 g, 33.5 mmol, 84% yield) as off white solid. MS (ESI): 374.2 ([M+H]⁺).

### c) 3-(4-(piperazin-1-yl)phenyl)piperidine-2,6-dione hydrochloride

To the stirred solution of tert-butyl 4-[4-(2,6-dioxo-3-piperidyl)phenyl]piperazine-1-carboxylate (13.1 g, 35.0 mmol) in DCM (50 mL) was added 4M HCl in dioxane (35.0 mmol) at 0° C. The mixture was then stirred at room temperature for 1 h. It was concentrated under reduced pressure and lyophilized to afford the title compound (10.8 g, 34.3 mmol, 98% yield, 99% purity) as off white solid. MS (ESI): 274.4 ([M+H]⁺).

### Ligase 71: 3-((6-(piperidin-4-yl)pyridin-3-yl)amino)piperidine-2,6-dione hydrochloride

### a) tert-butyl 4-(5-((2,6-dioxopiperidin-3-yl)amino)pyridin-2-yl)piperidine-1-carboxylate

tert-butyl 4-(5-aminopyridin-2-yl)piperidine-1-carboxylate (1 g, 3.61 mmol, Eq: 0.36) was dissolved in DMF (13 ml). Sodium bicarbonate (3.33 g, 39.7 mmol, Eq: 4) and 3-bromopiperidine-2,6-dione (1.9 g, 9.92 mmol, Eq: 1) were added. The reaction mixture was stirred at 90° C. over night. The crude residue was purified on silica gel (EtOAc) a 1:1 mixture of the title compound and the aniline starting material (2.17 g, 1.51 mmol, 15% yield) as a purple oil. This material was used for the next step without further purification.

### b) 3-((6-(piperidin-4-yl)pyridin-3-yl)amino)piperidine-2,6-dione hydrochloride

tert-butyl 4-(5-((2,6-dioxopiperidin-3-yl)amino)pyridin-2-yl)piperidine-1-carboxylate (1 g, 2.57 mmol, Eq: 1) was stirred with 4M HCl in dioxane (10 ml, 40 mmol, Eq: 15.5) in dioxane (35 ml) overnight at room temperature. The precipitated solid was filtered off, washed with ether and dried under high vacuum, affording the title compound as a 1:1 mixture with the aniline (554 mg, 1.71 mmol, 66% yield) as a light brown solid. It was used without purification in the next step.

### Ligase 72: 2-(4-(5-((2,6-dioxopiperidin-3-yl)oxy)pyridin-2-yl)piperidin-1-yl)acetic acid hydrochloride

### a) tert-butyl 2-(4-(5-((2,6-dioxopiperidin-3-yl)amino)pyridin-2-yl)piperidin-1-yl)acetate

To a solution of 3-((6-(piperidin-4-yl)pyridin-3-yl)oxy)piperidine-2,6-dione hydrochloride (216 mg, 663 μmol, Eq: 1), tert-butyl 2-bromoacetate (129 mg, 97.9 μl, 663 μmol, Eq: 1) and DIPEA (257 mg, 347 μl, 1.99 mmol, Eq: 3) in DMF (2.21 ml) was added potassium iodide (110 mg, 663 μmol, Eq: 1) and the reaction mixture was heated at 60° C. for 4 h. The reaction mixture was partitioned between ethyl acetate (40 ml) and 1 M sodium bicarbonate solutionmal (40 ml). The layers were separated. The aqueous layer was extracted with three 30-ml portions of ethyl acetate. The combined organic layers were washed with one 40-ml portion of water and brine, dried over sodium sulfate, filtered and concentrated in vacuo to yield the title compound (233 mg, 87%) as a light yellow solid.

### b) 2-(4-(5-((2,6-dioxopiperidin-3-yl)oxy)pyridin-2-yl)piperidin-1-yl)acetic acid hydrochloride

To a solution of tert-butyl 2-(4-(5-((2,6-dioxopiperidin-3-yl)oxy)pyridin-2-yl)piperidin-1-yl)acetate (0.233 g, 577 μmol, Eq: 1) in ethyl acetate (2.89 ml) was added 4 M hydrogen chloride solution in 1,4-dioxane (2.89 ml, 11.5 mmol, Eq: 20) at RT and stirring was continued for 16 h. The product was collected by filtration, washed with ethyl acetate and dried in vacuo to give the title compound (151 mg, 68% yield) as light yellow solid.

### Ligase 73: 4-[1-[1-(azetidin-3-yl)triazol-4-yl]ethoxy]-2-(2,6-dioxo-3-piperidyl)isoindoline-1,3-dione

### a) tert-butyl 3-(4-(1-((2-(2,6-dioxopiperidin-3-yl)-1,3-dioxoisoindolin-4-yl)oxy)ethyl)-1H-1,2,3-triazol-1-yl)azetidine-1-carboxylate

To a mixture of 2-(2,6-dioxo-3-piperidyl)-4-(1-methylprop-2-ynoxy)isoindoline-1,3-dione (150 mg, 459 umol) and tert-butyl 3-azidoazetidine-1-carboxylate (91 mg, 459 umol) in THF (6.0 mL) and water (1.0 mL) were added sodium ascorbate (182 mg, 919 umol) followed by copper sulfate (146 mg, 919 umol) at room temperature. The reaction mixture was stirred at room temperature for 16 h. Reaction mixture was added to water and extracted with ethyl acetate. The organic layer was washed with water, brine solution, dried over sodium sulfate, filtered and concentrated under reduced pressure. The crude residue was purified on silica (DCM/MeOH 0-15%) to afford the title compound (75.0 mg, 142 umol, 31% yield) as an off-white solid. MS (ESI): 525.3 ([M+H]⁺).

### b) 4-[1-[1-(azetidin-3-yl)triazol-4-yl]ethoxy]-2-(2,6-dioxo-3-piperidyl)isoindoline-1,3-dione

To a solution of tert-butyl 3-[4-[1-[2-(2,6-dioxo-3-piperidyl)-1,3-dioxo-isoindolin-4-yl]oxyethyl]triazol-1-yl]azetidine-1-carboxylate (75.0 mg, 142 umol) in DCM (1.0 mL) was added trifluoroacetic acid (163 mg, 1.4 mmol, 110 uL) at 0° C. under nitrogen atmosphere. The reaction mixture was warmed to room temperature and stirred for 1 hour. Reaction mixture was concentrated under reduced pressure and dried to afford title compound (68.0 mg, 85 umol, 59% yield) as a light brown semi solid (TFA salt). MS (ESI): 425.0 ([M+H]⁺).

### Ligase 74: 2-(2,6-dioxo-3-piperidyl)-4-[1-[1-(3-piperazin-1-ylpropyl)triazol-4-yl]ethoxy]isoindoline-1,3-dione

### a) tert-butyl 4-(3-(4-(1-((2-(2,6-dioxopiperidin-3-yl)-1,3-dioxoisoindolin-4-yl)oxy)ethyl)-1H-1,2,3-triazol-1-yl)propyl)piperazine-1-carboxylate

To a mixture of 2-(2,6-dioxo-3-piperidyl)-4-(1-methylprop-2-ynoxy)isoindoline-1,3-dione (500 mg, 1.53 mmol) and tert-butyl 4-(3-azidopropyl)piperazine-1-carboxylate (412 mg, 1.53 mmol) in DMSO (5.0 mL) and water (0.2 mL) were added sodium ascorbate (91 mg, 459 umol) followed by copper sulfate (24 mg, 153 umol) at room temperature. The reaction mixture was stirred at room temperature for 16 h. Reaction mixture was diluted with water and extracted with DCM. The organic layer was washed with water, brine solution, dried over sodium sulfate, filtered and concentrated under reduced pressure. The crude residue was purified on silica (DCM/MeOH 0-15%) to afford the title compound (660 mg, 1.0 mmol, 65% yield) as an off-white solid. MS (ESI): 596.1 ([M+H]⁺).

### b) 2-(2,6-dioxo-3-piperidyl)-4-[1-[1-(3-piperazin-1-ylpropyl)triazol-4-yl]ethoxy]isoindoline-1,3-dione

To a solution of tert-butyl 4-[3-[4-[1-[2-(2,6-dioxo-3-piperidyl)-1,3-dioxo-isoindolin-4-yl]oxyethyl]triazol-1-yl]propyl]piperazine-1-carboxylate (100 mg, 167 umol) in DCM (3.0 mL) was added trifluoroacetic acid (1.34 g, 11 mmol, 905 uL) at 0° C. under nitrogen atmosphere. The reaction mixture was warmed to room temperature and stirred for 3 h.

Reaction mixture was concentrated under reduced pressure and dried to afford title compound (110 mg, 166 umol, 98% yield) as a light brown solid (TFA salt). MS (ESI): 496.3 ([M+H]⁺).

### Ligase 75: 3-[4-[1-[2-(2,6-dioxo-3-piperidyl)-1,3-dioxo-isoindolin-4-yl]oxyethyl]triazol-1-yl]propanoic acid

### a) tert-butyl 3-(4-(1-((2-(2,6-dioxopiperidin-3-yl)-1,3-dioxoisoindolin-4-yl)oxy)ethyl)-1H-1,2,3-triazol-1-yl)propanoate

To a mixture of 2-(2,6-dioxo-3-piperidyl)-4-(1-methylprop-2-ynoxy)isoindoline-1,3-dione (230 mg, 704 umol) and tert-butyl 3-azidopropanoate (120 mg, 704 umol) in DMSO (3.0 mL) and water (0.5 mL) were added sodium ascorbate (41 mg, 211 umol) followed by copper sulfate (11.2 mg, 70 umol) at room temperature. The reaction mixture was stirred at room temperature for 16 h. Reaction mixture was diluted with water and extracted with 5% methanol in dichloromethane. The organic layer was washed with water, brine solution, dried over sodium sulfate, filtered and concentrated under reduced pressure. The crude residue was purified on silica (DCM/MeOH 0-10%) to afford the title compound (250 mg, 492 umol, 69% yield) as an off-white solid. MS (ESI): 498.3 ([M+H]⁺).

The title compound was prepared in analogy to Ligase 74 step b).

### Ligase 76: 2-(2,6-dioxo-3-piperidyl)-4-(1-methylprop-2-ynoxy)isoindoline-1,3-dione

To a mixture of 2-(2,6-dioxo-3-piperidyl)-4-hydroxy-isoindoline-1,3-dione (5.0 g, 18.2 mmol) and 1-methylprop-2-ynyl 4-methylbenzenesulfonate (4.91 g, 21.8 mmol) in DMF (50 mL) was added sodium carbonate (2.90 g, 27.3 mmol) at room temperature under nitrogen atmosphere. The reaction mixture was heated at 80° C. for 24 h. Reaction mixture was cooled to room temperature, diluted with water and extracted with ethylacetate. The organic layer was washed with brine solution, dried over sodium sulfate, filtered and concentrated under reduced pressure. The crude residue was purified silica (PE/EtOAc 0-100%) to afford the title compound (3.7 g, 11 mmol, 60% yield) as an off-white solid. MS (ESI): 327.2 ([M+H]⁺).

### Ligase 77: 5-(3,9-diazaspiro[5.5]undecan-3-yl)-2-(2,6-dioxo-3-piperidyl)isoindoline-1,3-dione

### a) tert-butyl 9-[2-(2,6-dioxo-3-piperidyl)-1,3-dioxo-isoindolin-5-yl]-3,9-diazaspiro[5.5]undecane-3-carboxylate

To a stirred solution of 2-(2,6-dioxo-3-piperidyl)-5-fluoro-isoindoline-1,3-dione (200 mg, 724 μmol, 1.0 eq) and tert-butyl 3,9-diazaspiro[5.5]undecane-3-carboxylate (184 mg, 724 μmol, 1.0 eq) in DMSO (1.5 mL) at room temperature was added DIPEA (187 mg, 253 μL, 1.45 mmol, 2.0 eq). The reaction mixture was stirred at 90° C. overnight. The reaction mixture was then poured into EtOAc/THF (1:1) and extracted sequentially with water and with brine. The organic phase was dried over Na₂SO₄ and concentrated in vacuo. The crude material was purified on silica column (heptane/EtOAc 0-100%) to afford the title compound (334 mg, 654 μmol, 90% yield) as a green solid. MS (ESI): 455.4 ([M+H—C₄H₈]⁺).

### b) 5-(3,9-diazaspiro[5.5]undecan-3-yl)-2-(2,6-dioxo-3-piperidyl)isoindoline-1,3-dione

To a stirred solution of tert-butyl 9-[2-(2,6-dioxo-3-piperidyl)-1,3-dioxo-isoindolin-5-yl]-3,9-diazaspiro[5.5]undecane-3-carboxylate (334 mg, 654 μmol, 1.0 eq) in dioxane (6 mL) at room temperature was added 4 M HCl in dioxane (477 mg, 454 μL, 13.1 mmol, 20.0 eq). The reaction mixture was stirred at room temperature for 2 h. The product was collected by filtration, washed with diethyl ether, and dried in vacuo to afford the title compound (336 mg, 752 μmol, 100% yield) as a white solid, hydrochloride salt. MS (ESI): 411.4 ([M+H]⁺).

### Example 1

### 4-[[7-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-7-oxo-heptyl]amino]-2-(2,6-dioxo-3-piperidyl)isoindoline-1,3-dione

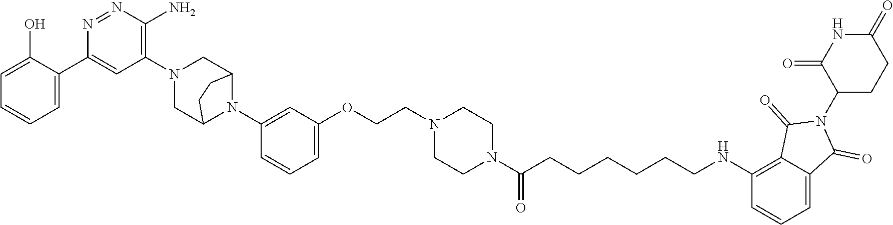 [SMILES: Nc1nnc(-c2ccccc2O)cc1N1CC2CCC(C1)N2c1cccc(OCCN2CCN(C(=O)CCCCCCNc3cccc4c3C(=O)N(C3CCC(=O)NC3=O)C4=O)CC2)c1]

### a) tert-butyl 4-[2-(3-bromophenoxy)ethyl]piperazine-1-carboxylate

To a solution of 3-bromophenol (7.51 g, 43.4 mmol, 1.0 eq), tert-butyl 4-(2-hydroxyethyl)piperazine-1-carboxylate (10 g, 43.4 mmol, 1.0 eq) and triphenylphospine (17.0 g, 65.1 mmol, 1.5 eq) in THF (100 mL) was added diethyl azodiacarboxylate (9.07 g, 52.1 mmol, 1.2 eq) at 0° C. The reaction mixture was stirred at 25° C. for 2 h. The mixture was diluted with water and extracted with EtOAc. The combined organic phase was washed with brine, dried over sodium sulfate, filtered and concentrated under reduced pressure. The residue was purified by prep-HPLC (FA) to afford the title compound (16 g, 41.5 mmol, 96% yield) as a light yellow oil. MS (ESI): 385.1/387.1 ([M+H]⁺).

### b) 1-[2-(3-bromophenoxy)ethyl]piperazine

To a solution of tert-butyl 4-[2-(3-bromophenoxy)ethyl]piperazine-1-carboxylate (16 g, 41.5 mmol, 1.0 eq) in 0.83 M HCl in dioxane (50 mL, 41.5 mmol, 1.0 eq) was stirred at 25° C. for 2 h. The reaction mixture was concentrated. The crude product was basified to pH=7 with NaHCO₃ solution, extrated with EtOAc and the combined organic layers were washed with brine. The combined organic layers were dried over sodium sulfate, concentrated to afford the title compound (11 g, 38.5 mmol, 93% yield) as a light yellow oil. MS (ESI): 286.8 ([M+H]⁺).

### c) benzyl 4-[2-(3-bromophenoxy)ethyl]piperazine-1-carboxylate

To a solution of 1-[2-(3-bromophenoxy)ethyl]piperazine (11 g, 38.5 mmol, 1.0 eq), triethylamine (16.1 mL, 115 mmol, 3.0 eq) in DCM (200 mL) was added benzyl chloroformate (7.9 g, 46.2 mmol, 1.2 eq). The reaction was stirred at 25° C. for 15 h. The mixture was diluted with water and extracted with EtOAc. The combined organic phase was washed with brine, dried over sodium sulfate, filtered and concentrated under reduced pressure. The residue was purified by prep-HPLC (FA) to afford the title compound (13.3 g, 31.7 mmol, 82% yield) as a yellow oil. MS (ESI): 418.0/420.0 ([M+H]⁺).

### d) tert-butyl 8-[3-[2-(4-benzyloxycarbonylpiperazin-1-yl)ethoxy]phenyl]-3,8-diazabicyclo[3.2.1]octane-3-carboxylate

A mixture of 3-Boc-3,8-diazabicyclo[3.2.1]octane (11695 mg, 55.0 mmol, 1.1 eq), Brettphos Pd G3 (2147 mg, 2.5 mmol, 0.05 eq), benzyl 4-[2-(3-bromophenoxy)ethyl]piperazine-1-carboxylate (21 g, 50 mmol, 1.0 eq) and potassium carbonate (13843 mg, 100 mmol, 2.0 eq) in tert-butanol (100 mL) was heated at 85° C. for 16 h under N₂. The mixture was filtered, then purified by prep-HPLC (base) to afford the title compound (12 g, 21.7 mmol, 33% yield) as a yellow oil. MS (ESI): 551.6 ([M+H]⁺).

### e) benzyl 4-[2-[3-(3,8-diazabicyclo[3.2.1]octan-8-yl)phenoxy]ethyl]piperazine-1-carboxylate

A mixture of tert-butyl 8-[3-[2-(4-benzyloxycarbonylpiperazin-1-yl)ethoxy]phenyl]-3,8-diazabicyclo[3.2.1]octane-3-carboxylate (3000 mg, 5.45 mmol, 1.0 eq) and 4 M HCl in dioxane (20 mL, 5.45 mmol, 1.0 eq) in methanol (100 mL) was heated at 85° C. for 16 h under N₂. The mixture was filtered, then purified by prep-HPLC (base) to afford the title compound (2.5 g, 5.55 mmol, 101% yield) as a yellow oil.

### f) benzyl 4-[2-[3-[3-(3-amino-6-chloro-pyridazin-4-yl)-3,8-diazabicyclo[3.2.1]octan-8-yl]phenoxy]ethyl]piperazine-1-carboxylate

A mixture of 4-bromo-6-chloro-pyridazin-3-amine (1279 mg, 6.1 mmol, 1.3 eq), benzyl 4-[2-[3-(3,8-diazabicyclo[3.2.1]octan-8-yl)phenoxy]ethyl]piperazine-1-carboxylate hydrochloride (2.3 g, 4.7 mmol, 1.0 eq) and triethylamine (1.32 mL, 9.4 mmol, 2.0 eq) in DMF (10 mL) was heated at 85° C. for 16 h. The mixture was poured into water, extracted with EtOAc, washed with brine, concentrated in vacuum and the residue was purified on silica column (DCM/EtOAc=2:1) to afford the title compound (2 g, 3.4 mmol, 69% yield) as a light yellow solid. MS (ESI): 578.3 ([M+H]⁺).

### g) benzyl 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate

A mixture of 2-hydroxyphenylboronic acid (35.7 mg, 0.26 mmol, 1.5 eq), Brettphos Pd G3 (14.8 mg, 0.02 mmol, 0.1 eq), sodium carbonate (36.6 mg, 0.35 mmol, 2.0 eq) and benzyl 4-[2-[3-[3-(3-amino-6-chloro-pyridazin-4-yl)-3,8-diazabicyclo[3.2.1]octan-8-yl]phenoxy]ethyl]piperazine-1-carboxylate (0.1 g, 0.17 mmol, 1.0 eq) in tert-butanol (3 mL) was stirred under nitrogen at 90° C. for 16 h. The mixture was purified on silica column to afford the title compound (60 mg, 0.09 mmol, 54% yield) as a yellow oil. MS (ESI): 636.5 ([M+H]⁺).

### h) 2-[6-amino-5-[8-[3-(2-piperazin-1-ylethoxy)phenyl]-3,8-diazabicyclo[3.2.1]octan-3-yl]pyridazin-3-yl]phenol

A mixture of benzyl 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate (400 mg, 0.63 mmol, 1.0 eq) and palladium on carbon (0.07 mL, 0.06 mmol, 0.1 eq) in methanol (10 mL) was stirred under H₂ at 20° C. for 16 h. The mixture was filtered, concentrated and purified on silica column (TFA) to afford the title compound (316 mg, 0.51 mmol, 82% yield) as a yellow solid, 2,2,2-trifluoroacetic acid salt. MS (ESI): 387.3 ([M+H]⁺).

### i) 4-[[7-[4-[2-[3-[3-[3-amino-6-(2-hydroxyphenyl)pyridazin-4-yl]-3,8-diazabicyclo[3.2.1] octan-8-yl]phenoxy]ethyl]piperazin-1-yl]-7-oxo-heptyl]amino]-2-(2,6-dioxo-3-piperidyl) isoindoline-1,3-dione

A mixture of Ligase 1 (10 mg, 0.02 mmol, 1.0 eq), 2-[6-amino-5-[8-[3-(2-piperazin-1-ylethoxy)phenyl]-3,8-diazabicyclo[3.2.1]octan-3-yl]pyridazin-3-yl]phenol (12.5 mg, 0.02 mmol, 1.0 eq), HATU (14 mg, 0.04 mmol, 1.5 eq) and DIPEA (6.5 mg, 0.05 mmol, 2.0 eq) in DMF (5 mL) was stirred at 25° C. for 2 h. The mixture was concentrated in vacuum, then purified by prep-HPLC to afford the title compound (4.4 mg, 0.005 mmol, 18% yield) as a yellow solid. MS (ESI): 885.6 ([M+H]⁺).

### Example 2

### 4-[4-[3-[9-[3-amino-6-(2-hydroxyphenyl)pyridazin-4-yl]-1-oxa-4,9-diazaspiro[5.5]undecan-4-yl]-3-oxo-propyl]-1-piperidyl]-2-(2,6-dioxo-3-piperidyl)isoindoline-1,3-dione

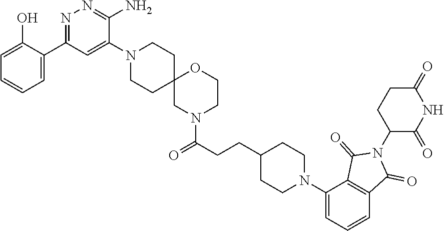 [SMILES: Nc1nnc(-c2ccccc2O)cc1N1CCC2(CC1)CN(C(=O)CCC1CCN(c3cccc4c3C(=O)N(C3CCC(=O)NC3=O)C4=O)CC1)CCO2]

### a) tert-butyl 9-(3-amino-6-chloro-pyridazin-4-yl)-1-oxa-4,9-diazaspiro[5.5]undecane-4-carboxylate

To a stirred solution of 4-bromo-6-chloropyridazin-3-amine (600 mg, 2.88 mmol, 1.0 eq) and tert-butyl 1-oxa-4,9-diazaspiro[5.5]undecane-4-carboxylate (812 mg, 3.17 mmol, 1.1 eq) in DMA (8 mL) was added potassium carbonate (1.19 g, 8.64 mmol, 3.0 eq). The reaction mixture was heated to 110° C. and stirred for 20 h. The reaction mixture was poured in water and extracted with EtOAc. The organic layers were combined, washed with sat NaHCO₃, WATER and brine. The organic layers were dried over Na₂SO₄ and concentrated in vacuo. The crude material was purified on silica column (heptane/EtOAc 0-100%) to afford the title compound (1.02 g, 2.66 mmol, 92% yield) as a light brown solid. MS (ESI): 384.2 ([M+H]⁺).

### b) tert-butyl 9-[3-amino-6-(2-hydroxyphenyl)pyridazin-4-yl]-1-oxa-4,9-diazaspiro[5.5]undecane-4-carboxylate

A suspension of tert-butyl 9-(3-amino-6-chloropyridazin-4-yl)-1-oxa-4,9-diazaspiro[5.5]undecane-4-carboxylate (1.02 g, 2.66 mmol, 1.0 eq), (2-hydroxyphenyl)boronic acid (733 mg, 5.31 mmol, 2.0 eq), K₂CO₃ (1.1 g, 7.97 mmol, 3.0 eq) and Ruphos Pd G3 (111 mg, 133 μmol, 0.05 eq) in a mixture of degassed dioxane (10 mL) and water (1 mL) was stirred at 120° C. for 16 h under argon. The reaction mixture was poured in saturated NaHCO₃ and extracted with EtOAc. The organic layers were combined, washed with water and brine. The organic layers were dried over Na₂SO₄ and concentrated in vacuo. The crude material was purified on silica column (heptane/EtOAc 0-100%) to afford the title compound (860 mg, 1.95 mmol, 73% yield) as a light brown solid. MS (ESI): 442.4 ([M+H]⁺).

### c) 2-[6-amino-5-(1-oxa-4,9-diazaspiro[5.5]undecan-9-yl)pyridazin-3-yl]phenol

To a cooled (0° C.) solution of tert-butyl 9-(3-amino-6-(2-hydroxyphenyl)pyridazin-4-yl)-1-oxa-4,9-diazaspiro[5.5]undecane-4-carboxylate (330 mg, 747 μmol, 1.0 eq) in DCM (4 mL) was added 4M HCl in dioxane (934 μL, 3.74 mmol, 5.0 eq). The reaction mixture was allowed to reach room temperature and stirred for 16 h. The reaction mixture was concentrated in vacuo to afford the title compound (303 mg, 732 μmol, 97% yield) as a white solid, hydrochloride salt. MS (ESI): 342.3 ([M+H]⁺).

### d) 4-[4-[3-[9-[3-amino-6-(2-hydroxyphenyl)pyridazin-4-yl]-1-oxa-4,9-diazaspiro[5.5]undecan-4-yl]-3-oxo-propyl]-1-piperidyl]-2-(2,6-dioxo-3-piperidyl)isoindoline-1,3-dione

Ligase 2 (16 mg, 38.7 μmol, 1.0 eq), HATU (18.4 mg, 48.4 μmol, 1.25 eq) and DIPEA (40 mg, 54.1 μL, 310 μmol, 8.0 eq) were combined with DMF (1 mL). Then 2-(6-amino-5-(1-oxa-4,9-diazaspiro[5.5]undecan-9-yl)pyridazin-3-yl)phenol hydrochloride (15.4 mg, 40.6 μmol, 1.05 eq) was added. The reaction mixture was stirred at room temperature for 2 h, concentrated in vacuo and purified by prep-HPLC to afford the title compound (16 mg, 21.7 μmol, 56% yield) as a yellow solid. MS (ESI): 737.5 ([M+H]⁺).

### Example 3

### 4-[[1-[9-[4-[4-[3-[3-amino-6-(2-hydroxyphenyl)pyridazin-4-yl]oxy-1-piperidyl]phenyl]piperazin-1-yl]-9-oxo-nonyl]triazol-4-yl]methylamino]-2-(2,6-dioxo-3-piperidyl)isoindoline-1,3-dione

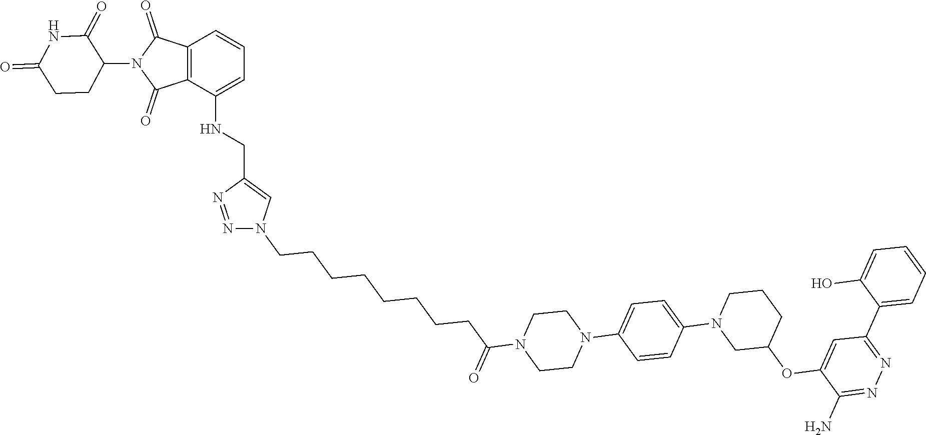 [SMILES: Nc1nnc(-c2ccccc2O)cc1OC1CCCN(c2ccc(N3CCN(C(=O)CCCCCCCCn4cc(CNc5cccc6c5C(=O)N(C5CCC(=O)NC5=O)C6=O)nn4)CC3)cc2)C1]

### a) tert-butyl 4-(4-(3-hydroxypiperidin-1-yl)phenyl)piperazine-1-carboxylate

A sealed tube was charged with tert-butyl 4-(4-iodophenyl)piperazine-1-carboxylate (2.5 g, 6.44 mmol), piperidin-3-ol (1.63 g, 16.1 mmol), potassium phosphate tribasic anhydrous (4.1 g, 19.3 mmol) and L-proline, 99% (370 mg, 3.22 mmol) and DMF (30 mL). The reaction mixture was purged with nitrogen for 15 min and was added copper (I) iodide (613 mg, 3.22 mmol), purging was continued for another 5 min, and the reaction mixture was heated to 100° C. for 16 h. The reaction was cooled to room temperature passed through celite bed, washed with EtOAc and concentrated under reduced pressure. The crude residue was purified on silica column (PE/EtOAc 0-90%) to afford the title compound (1.3 g, 3.60 mmol, 55% yield) as an off-white solid. MS (ESI): 362.3 ([M+H]⁺).

### b) tert-butyl 4-(4-(3-((3-amino-6-chloropyridazin-4-yl)oxy)piperidin-1-yl)phenyl)piperazine-1-carboxylate

To a solution of tert-butyl 4-[4-(3-hydroxy-1-piperidyl)phenyl]piperazine-1-carboxylate (1.2 g, 3.32 mmol) in dimethylformamide (20 mL) at 0° C. was added sodium hydride, 60% dispersion in mineral oil (229 mg, 9.96 mmol) and the reaction mixture was heated to 55° C. for 1 h. This reaction mixture was cooled to room temperature and was added 4-bromo-6-chloropyridazin-3-amine (1.73 g, 8.3 mmol) in dimethylformamide (10 mL) and heated at 80° C. for 4 h. The reaction mixture was quenched with saturated ammonium chloride solution and extracted with EtOAc. The organic layer was washed with water, brine solution, dried over sodium sulfate, filtered and concentrated under reduced pressure. The crude residue was purified on silica column (PE/EtOAc 20-80%) to afford the title compound (0.6 g, 1.2 mmol, 36% yield) as light brown solid. MS (ESI): 489.2 ([M+H]⁺).

### c) tert-butyl 4-(4-(3-((3-amino-6-(2-hydroxyphenyl)pyridazin-4-yl)oxy)piperidin-1-yl)phenyl)piperazine-1-carboxylate

In a sealed tube, tert-butyl 4-[4-[3-(3-amino-6-chloro-pyridazin-4-yl)oxy-1-piperidyl]phenyl]piperazine-1-carboxylate (1.0 g, 2.04 mmol), (2-hydroxyphenyl)boronic acid (338 mg, 2.45 mmol), [1,1′-bis(diphenylphosphino)ferrocene]dichloropalladium(II), complex with DCM (167 mg, 204.5 μmol) and potassium carbonate (847.87 mg, 6.13 mmol) followed by 1,4-dioxane (14 mL) and water (2 mL) were added and the reaction mixture was degassed with nitrogen for 10 min. The reaction mixture was heated to 120° C. for 12 h. The reaction was cooled to ambient temperature passed through celite bed, washed with EtOAc and concentrated under reduced pressure. The crude residue was purified on silica column (PE/EtOAc 10-80%) to afford the title compound (0.52 g, 951 μmol, 46% yield) as a brown solid. MS (ESI): 547.3 ([M+H]⁺).

### d) 2-(6-amino-5-((1-(4-(piperazin-1-yl)phenyl)piperidin-3-yl)oxy)pyridazin-3-yl)phenol

To a solution of tert-butyl 4-[4-[3-[3-amino-6-(2-hydroxyphenyl)pyridazin-4-yl]oxy-1-piperidyl]phenyl]piperazine-1-carboxylate (125 mg, 228 μmol) in DCM (2 mL) at 0° C. was added trifluoro acetic acid (260 mg, 2.29 mmol, 176.16 μL) and it was stirred at room temperature for 3 h. The reaction mixture was concentrated under reduced pressure, and co-distilled with DCM to afford the title compound (100 mg, 223 μmol, 97% yield) as a brown colored semi solid. MS (ESI): 447.5 ([M+H]⁺).

### e) 4-[[1-[9-[4-[4-[3-[3-amino-6-(2-hydroxyphenyl)pyridazin-4-yl]oxy-1-piperidyl]phenyl]piperazin-1-yl]-9-oxo-nonyl]triazol-4-yl]methylamino]-2-(2,6-dioxo-3-piperidyl)isoindoline-1,3-dione

A screw cap vial (8 mL) was charged with 2-[6-amino-5-[[1-(4-piperazin-1-ylphenyl)-3-piperidyl]oxy]pyridazin-3-yl]phenol (60 mg, 134 umol), Ligase 3 (68.6 mg, 134 umol), DMF (1.0 mL) and to this reaction mixture was added DIPEA (86.8 mg, 117 uL, 671 umol) followed by HATU (76.6 mg, 201 umol) at room temperature and the reaction mixture was kept on an orbital shaker for 16 hours. The reaction mixture was diluted with water and extracted with EtOAc. The volatiles were removed under Genevac at 50° C. The crude residue was purified by prep-HPLC to afford the title compound (13.0 mg, 13.8 μmol, 10% yield) as a light yellow solid, trifluoro acetic acid salt. MS (ESI): 940.0 ([M+H]+).

### Example 4

### 4-[[1-[12-[4-[4-[3-[3-amino-6-(2-hydroxyphenyl)pyridazin-4-yl]oxy-1-piperidyl]phenyl]piperazin-1-yl]-12-oxo-dodecyl]triazol-4-yl]methylamino]-2-(2,6-dioxo-3-piperidyl)isoindoline-1,3-dione

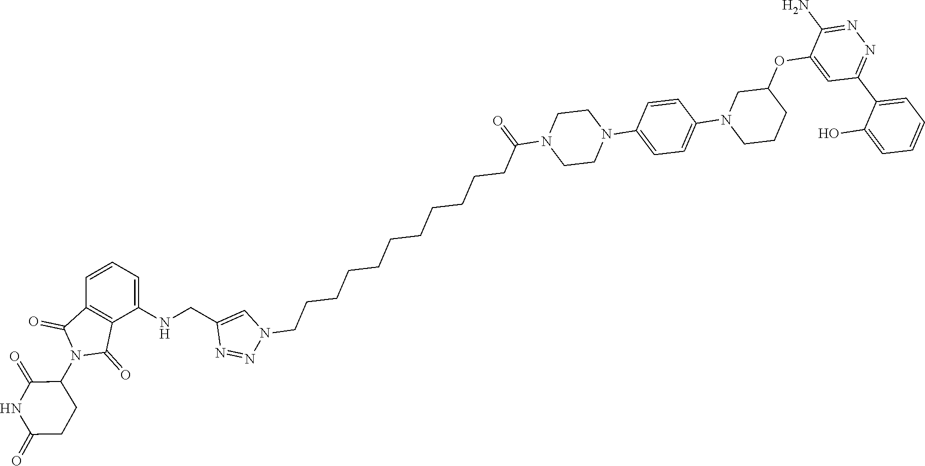 [SMILES: Nc1nnc(-c2ccccc2O)cc1OC1CCCN(c2ccc(N3CCN(C(=O)CCCCCCCCCCCn4cc(CNc5cccc6c5C(=O)N(C5CCC(=O)NC5=O)C6=O)nn4)CC3)cc2)C1]

The title compound (15.1 mg, 15.4 μmol, 11% yield) was prepared in analogy to example 3 step e using Ligase 4 as a yellow solid, trifluoro acetic acid salt. MS (ESI): 982.0 ([M+H]+).

### Example 5

### 4-[[7-[4-[2-[3-[7-[3-amino-6-(2-hydroxyphenyl)pyridazin-4-yl]-4,7-diazaspiro[2.5]octan-4-yl]phenoxy]ethyl]piperazin-1-yl]-7-oxo-heptyl]amino]-2-(2,6-dioxo-3-piperidyl)isoindoline-1,3-dione

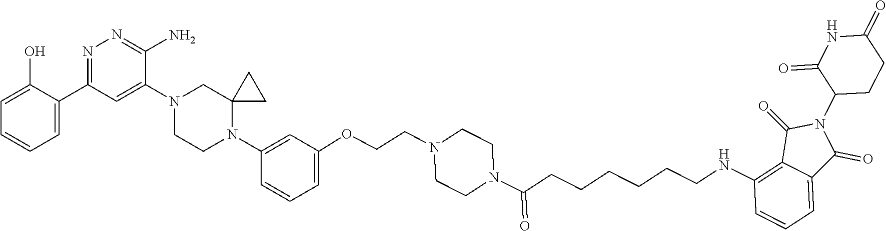 [SMILES: Nc1nnc(-c2ccccc2O)cc1N1CCN(c2cccc(OCCN3CCN(C(=O)CCCCCCNc4cccc5c4C(=O)N(C4CCC(=O)NC4=O)C5=O)CC3)c2)C2(CC2)C1]

### a) benzyl 4-[3-[2-(4-tert-butoxycarbonylpiperazin-1-yl)ethoxy]phenyl]-47-diazaspiro[2.5]octane-7-carboxylate

A suspension of tert-butyl 4-(2-(3-bromophenoxy)ethyl)piperazine-1-carboxylate (188 mg, 487 μmol, 1.0 eq), benzyl 4,7-diazaspiro[2.5]octane-7-carboxylate (120 mg, 487 μmol, 1.0 eq), sodium tert-butoxide (60.9 mg, 633 μmol, 1.3 eq), palladium (II) acetate (5.47 mg, 24.4 μmol, 0.05 eq) and Ruphos (22.7 mg, 48.7 μmol, 0.1 eq) in degassed dioxane (5 mL) was stirred at 100° C. for 20 h under argon. The reaction mixture was poured in saturated NaHCO₃ and extracted with EtOAc. The organic layers were combined and washed with WATER and brine. The organic layers were dried over Na₂SO₄ and concentrated in vacuo. The crude material was purified on silica column (heptane/EtOAc 0 to 100%) to afford the title compound (80 mg, 145 μmol, 26% yield) as a yellow solid. MS (ESI): 551.4 ([M+H]⁺).

### b) tert-butyl 4-[2-[3-(4,7-diazaspiro[2.5]octan-4-yl)phenoxy]ethyl]piperazine-1-carboxylate

To a solution of benzyl 4-(3-(2-(4-(tert-butoxycarbonyl)piperazin-1-yl)ethoxy)phenyl)-4,7-diazaspiro[2.5]octane-7-carboxylate (140 mg, 254 μmol, 1.0 eq) in methanol (2 mL) was added 10% palladium on charcoal (27.1 mg, 25.4 μmol, 0.10 eq). The reaction mixture was vigorously stirred at room temperature for 2 h under H₂ (baloon). The catalyst was collected by filtration, washing with methanol. The filtrate was concentrated to afford the title compound (105 mg, 252 μmol, 99% yield) as alight brown oil. MS (ESI): 417.4 ([M+H]⁺).

### c) tert-butyl 4-[2-[3-[7-(3-amino-6-chloro-pyridazin-4-yl)-4,7-diazaspiro[2.5]octan-4-yl]phenoxy]ethyl]piperazine-1-carboxylate

To a stirred solution of 4-bromo-6-chloropyridazin-3-amine (25 mg, 120 μmol, 1.0 eq) and tert-butyl 4-(2-(3-(4,7-diazaspiro[2.5]octan-4-yl)phenoxy)ethyl)piperazine-1-carboxylate (50 mg, 120 μmol, 1.0 eq) in DMA (2 mL) was added potassium carbonate (33.2 mg, 240 μmol, 2.0 eq). The reaction mixture was heated to 110° C. and stirred for 20 h. The reaction mixture was poured in water and extracted with EtOAc. The organic layers were combined, washed with saturated NaHCO₃, water and brine. The organic layers were dried over Na₂SO₄ and concentrated in vacuo. The crude material was purified on silica column (DCM/MeOH 0-10%) to afford the title compound (20 mg, 37 μmol, 31% yield) as a brown oil. MS (ESI): 544.3 ([M+H]⁺).

### d) tert-butyl 4-[2-[3-[7-[3-amino-6-(2-hydroxyphenyl)pyridazin-4-yl]-4,7-diazaspiro[2.5]octan-4-yl]phenoxy]ethyl]piperazine-1-carboxylate

A suspension of tert-butyl 4-(2-(3-(7-(3-amino-6-chloropyridazin-4-yl)-4,7-diazaspiro[2.5]octan-4-yl)phenoxy)ethyl)piperazine-1-carboxylate (30 mg, 55.1 μmol, 1.0 eq), (2-hydroxyphenyl)boronic acid (19 mg, 138 μmol, 2.5 eq) and potassium carbonate (22.9 mg, 165 μmol, 3.0 eq), followed by RuPhos Pd G3 (1.38 mg, 1.65 μmol, 0.03 eq) in a mixture of degassed dioxane (10 mL) and water (1 mL) was stirred at 120° C. for 16 h. The reaction mixture was poured in saturated NaHCO₃ and extracted with EtOAc. The organic layers were combined, washed with water and brine. The organic layers were dried over Na₂SO₄ and concentrated in vacuo. The crude material was purified on silica column (DCM/MeOH 0-5%) to afford the title compound (15 mg, 22 μmol, 41% yield) as a yellow solid. MS (ESI): 602.5 ([M+H]⁺).

### e) 2-[6-amino-5-[4-[3-(2-piperazin-1-ylethoxy)phenyl]-4,7-diazaspiro[2.5]octan-7-yl]pyridazin-3-yl]phenol hydrochloride

To a cooled (0° C.) solution of tert-butyl 4-(2-(3-(7-(3-amino-6-(2-hydroxyphenyl)pyridazin-4-yl)-4,7-diazaspiro[2.5]octan-4-yl)phenoxy)ethyl)piperazine-1-carboxylate (7 mg, 11.6 μmol, 1.0 eq) in DCM (0.5 mL) was added 4M HCl in dioxane (8.72 μL, 34.9 μmol, 3.0 eq). The reaction mixture was allowed to reach room temperature and stirred for 20 h. The reaction mixture was concentrated in vacuo to afford the title compound (5 mg, 10.0 μmol μmol, 86% yield) as a white sokid, hydrochloride salt. MS (ESI): 502.4 ([M+H]⁺).

### f) 4-[[7-[4-[2-[3-[7-[3-amino-6-(2-hydroxyphenyl)pyridazin-4-yl]-4,7-diazaspiro[2.5]octan-4-yl]phenoxy]ethyl]piperazin-1-yl]-7-oxo-heptyl]amino]-2-(2,6-dioxo-3-piperidyl)isoindoline-1,3-dione

Ligase 1 (3.92 mg, 9.76 μmol, 1.05 eq), HATU (5.3 mg, 13.9 μmol, 1.5 eq) and DIPEA (12 mg, 16.2 μL, 92.9 μmol, 10.0 eq) were combined with DMF (0.5 mL). 2-(6-amino-5-(4-(3-(2-(piperazin-1-yl)ethoxy)phenyl)-4,7-diazaspiro[2.5]octan-7-yl)pyridazin-3-yl)phenol, hydrochloride salt (5 mg, 9.29 μmol, 1.0 eq) was added. The reaction mixture was stirred at 22° C. for 2 h. The crude reaction mixture was concentrated in vacuo and directly purified by prep-HPLC to afford the title compound (2 mg, 2.2 μmol, 22% yield) as a yellow solid. MS (ESI): 885.5 ([M+H]⁺).

### Example 6

### 4-[[9-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-9-oxo-nonyl]amino]-2-(2,6-dioxo-3-piperidyl)isoindoline-1,3-dione

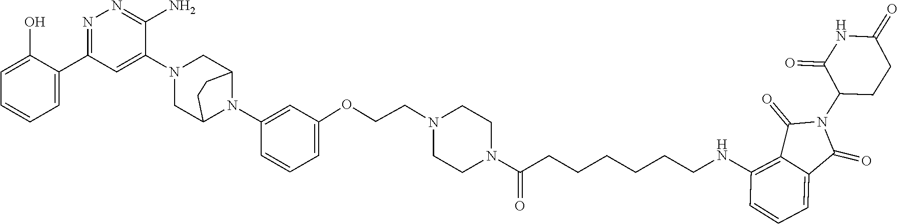 [SMILES: Nc1nnc(-c2ccccc2O)cc1N1CC2CCC(C1)N2c1cccc(OCCN2CCN(C(=O)CCCCCCNc3cccc4c3C(=O)N(C3CCC(=O)NC3=O)C4=O)CC2)c1]

### a) 9-[[2-(2,6-dioxo-3-piperidyl)-1,3-dioxo-isoindolin-4-yl]amino]nonanoic acid

A mixture of 2-(2,6-dioxo-3-piperidyl)-4-fluoro-isoindoline-1,3-dione (160 mg, 0.58 mmol, 1.0 eq), 9-aminononanoic acid (110 mg, 0.64 mmol, 1.1 eq) and DIPEA (225 mg, 1.74 mmol, 3.0 eq) in DMSO (5 mL) was stirred at 80° C. for 12 h. The mixture was purified on silica column to afford the title compound (40 mg, 0.09 mmol, 16% yield) as a white solid. MS (ESI): 430.3 ([M+H]⁺).

### b) 4-[[9-[4-[2-[3-[(1R,5S)-3-[3-amino-6-(2-hydroxyphenyl)pyridazin-4-yl]-3,8-diazabicyclo[3.2.1]octan-8-yl]phenoxy]ethyl]piperazin-1-yl]-9-oxo-nonyl]amino]-2-(2,6-dioxo-3-piperidyl)isoindoline-1,3-dione

A mixture of 2-[6-amino-5-[(1R,5S)-8-[3-(2-piperazin-1-ylethoxy)phenyl]-3,8-diazabicyclo[3.2.1]octan-3-yl]pyridazin-3-yl]phenol (30 mg, 0.06 mmol, 1.0 eq), Ligase 5 (33 mg, 0.08 mmol, 1.3 eq), HATU (34 mg, 0.09 mmol, 1.5 eq) and DIPEA (0.03 mL, 0.18 mmol, 3.0 eq) in DMF (5 mL) was stirred at 25° C. for 2 h. The mixture was concentrated in vacuum, then purified by prep-HPLC (FA) to afford the title compound (3.2 mg, 0.003 mmol, 5% yield) as a yellow solid. MS (ESI): 913.5 ([M+H]⁺).

### Example 7

### 4-[[1-[12-[9-[3-amino-6-(2-hydroxyphenyl)pyridazin-4-yl]-1-oxa-4,9-diazaspiro[5.5]undecan-4-yl]-12-oxo-dodecyl]triazol-4-yl]methylamino]-2-(2,6-dioxo-3-piperidyl)isoindoline-1,3-dione

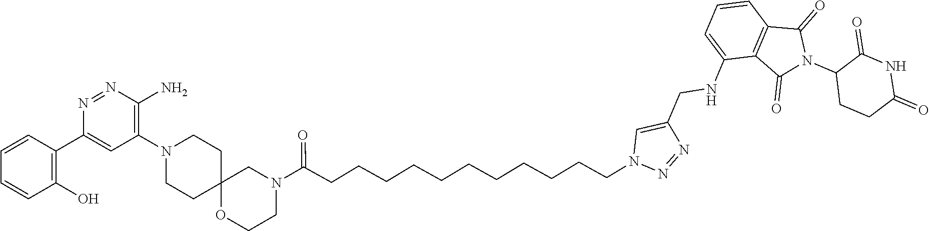 [SMILES: Nc1nnc(-c2ccccc2O)cc1N1CCC2(CC1)CN(C(=O)CCCCCCCCCCCn1cc(CNc3cccc4c3C(=O)N(C3CCC(=O)NC3=O)C4=O)nn1)CCO2]

A screw cap vial (8 mL) was charged with 2-(6-amino-5-(1-oxa-4,9-diazaspiro[5.5]undecan-9-yl)pyridazin-3-yl)phenol (20 mg, 0.053 mmol), Ligase 4 (29 mg, 0.053 mmol), DMF (0.5 mL). DIPEA (34.20 mg, 0.046 mL, 0.264 mmol) followed by HATU (30.1 mg, 0.079 mmol) were added and the reaction mixture was stirred for 16 h at room temperature. It was diluted with water and extracted with EtOAc. The volatiles were removed. The crude residue was purified by prep-HPLC to afford the title compound compound (11.2 mg, 11.7 μmol, 22% yield) as an off-white solid, trifluoroacetic acid salt. MS (ESI): 876 ([M+H]⁺).

### Example 8

### 4-[[1-[15-[4-[4-[3-[3-amino-6-(2-hydroxyphenyl)pyridazin-4-yl]oxy-1-piperidyl]phenyl]piperazin-1-yl]-15-oxo-pentadecyl]triazol-4-yl]methylamino]-2-(2,6-dioxo-3-piperidyl)isoindoline-1,3-dione

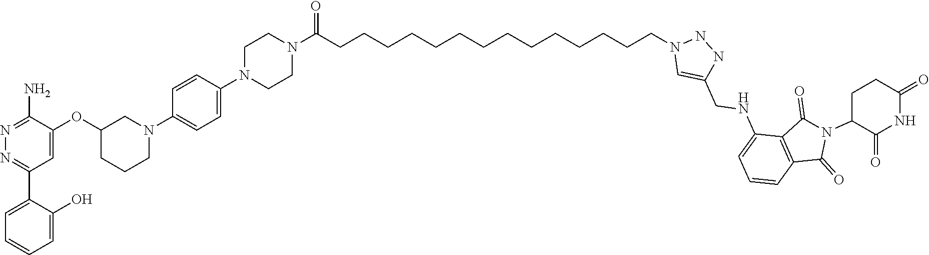 [SMILES: Nc1nnc(-c2ccccc2O)cc1OC1CCCN(c2ccc(N3CCN(C(=O)CCCCCCCCCCCCCCN4C=C(CNc5cccc6c5C(=O)N(C5CCC(=O)NC5=O)C6=O)NN4)CC3)cc2)C1]

The title compound (7.3 mg, 6.24 μmol, 13% yield) was prepared in analogy to example 3 step e using Ligase 6 as a light yellow solid, trifluoroacetic acid salt. MS (ESI): 1023.9 ([M+H]+).

### Example 9

### rac-4-[[1-[4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoyl]-4-piperidyl]oxy]-2-(2,6-dioxo-3-piperidyl)isoindoline-1,3-dione

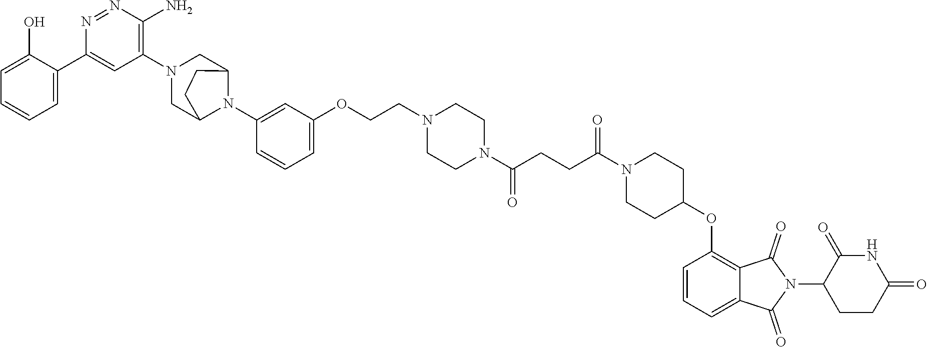 [SMILES: Nc1nnc(-c2ccccc2O)cc1N1CC2CCC(C1)N2c1cccc(OCCN2CCN(C(=O)CCC(=O)N3CCC(Oc4cccc5c4C(=O)N(C4CCC(=O)NC4=O)C5=O)CC3)CC2)c1]

### a) benzyl 4-(2-hydroxyethyl)piperazine-1-carboxylate

To a stirred solution of 2-(piperazin-1-yl)ethan-1-ol (4.5 g, 4.24 ml, 34.6 mmol, 1.0 eq) and Et₃N (3.5 g, 4.82 ml, 34.6 mmol, 1.0 eq) in THF (150 mL) was added dropwise benzyl carbonochloridate (5.9 g, 4.87 ml. 34.6 mmol. 1.0 ea) at 0-5° C. over 15 min. The reaction mixture was stirred for 35 min at 0-5° C. and was then allowed to warm to room temperature. The reaction mixture was then stirred for a further 5 h. A white suspension resulted. The reaction mixture was partitioned between EtOAc and water. The layers were separated. The organic layer was washed with brine, dried over anhydrous sodium sulfate, and concentrated in vacuo. The crude material was purified on silica column (DCM/MeOH 0-5%) to afford the title compound (3.04 g, 11.5 mmol, 33% yield) as a colourless oil. MS (ESI): 265.0 ([M+H]⁺).

### b) tert-butyl 8-(3-benzyloxyphenyl)-3,8-diazabicyclo[3.2.1]octane-3-carboxylate

To a stirred suspension of 1-(benzyloxy)-3-bromobenzene (2 g, 7.6 mmol, 1.0 eq) and tert-butyl 3,8-diazabicyclo[3.2.1]octane-3-carboxylate (1.69 g, 7.98 mmol, 1.05 eq) in t-BuOH (12.3 mL) at room temperature was added K₂CO₃ (2.1 g, 15.2 mmol, 2.0 eq). The reaction was degassed with argon for 5 min. RuPhos Pd G3 (636 mg, 760 μmol, 0.1 eq) was then added. The reaction mixture was stirred at 120° C. overnight, poured into EtOAc/THF (2:1) and washed sequentially with water and with brine. The organic layer was dried over Na₂SO₄ and concentrated in vacuo. The crude material was purified on silica column (heptane/EtOAc 0-15%) to afford the title compound (2.54 g, 6.4 mmol, 85% yield) as a yellow oil. MS (ESI): 395.4 ([M+H]⁺).

### c) tert-butyl 8-(3-hydroxyphenyl)-3,8-diazabicyclo[3.2.1]octane-3-carboxylate

A 250 ml two-necked round-bottomed flask was charged with tert-butyl 8-(3-benzyloxyphenyl)-3,8-diazabicyclo[3.2.1]octane-3-carboxylate (2.54 g, 6.44 mmol, 1.0 eq), ammonium formate (8.12 g, 129 mmol, 20 eq) and methanol (150 mL). The flask was degassed with argon. The catalyst 10% Pd on charcoal (685 mg, 644 μmol, 0.1 eq) was added. The reaction mixture was heated at 70° C. for 15 h. The catalyst was removed by filtration and washed with methanol. The filtrate was concentrated in vacuo. The residue was partitioned between EtOAc/THF (1:1) and water. The layers were separated. The organic layer was washed with brine, dried over anhydrous sodium sulfate, and concentrated in vacuo to afford the title compound (1.89 g, 6.2 mmol, 96% yield) as a white solid. MS (ESI): 305.3 ([M+H]⁺).

### d) tert-butyl 8-[3-[2-(4-benzyloxycarbonylpiperazin-1-yl)ethoxy]phenyl]-3,8-diazabicyclo[3.2.1]octane-3-carboxylate

To a stirred solution of tert-butyl 8-(3-hydroxyphenyl)-3,8-diazabicyclo[3.2.1]octane-3-carboxylate (1.892 g, 6.22 mmol, 1.0 eq), benzyl 4-(2-hydroxyethyl)piperazine-1-carboxylate (1.81 g, 6.84 mmol, 1.1 eq) and di-tert-butyl azodicarboxylate (1.57 g, 6.84 mmol, 1.1 eq) in THF (27 mL) at room temperature was added triphenylphosphine (1.79 g, 6.84 mmol, 1.1 eq). The reaction mixture was stirred over the weekend, partitioned between EtOAc/THF (1:1) and 0.5 M aqueous sodium hydroxide solution. The aqueous layer was extracted with EtOAc. The combined organic layers were washed with brine, dried over anhydrous sodium sulfate, and concentrated in vacuo. The crude material was purified on silica column (heptane/EtOAc 0-100%) afford the title compound (3.81 g, 5.5 mmol, 89% yield) as a white solid. MS (ESI): 551.5 ([M+H]⁺).

### e) benzyl 4-[2-[3-(3,8-diazabicyclo[3.2.1]octan-8-yl)phenoxy]ethyl]piperazine-1-carboxylate

To a stirred solution of tert-butyl 8-[3-[2-(4-benzyloxycarbonylpiperazin-1-yl)ethoxy]phenyl]-3,8-diazabicyclo[3.2.1]octane-3-carboxylate (3.81 g, 4.84 mmol, 1.0 eq) in DCM (10 mL) at room temperature was added TFA (3.7 ml, 48.4 mmol, 10 eq). The reaction mixture was stirred for 3 h. The solvent was concentrated in vacuo. The residue was partitioned between EtOAc and 1 M aqueous hydrochloric acid. The aqueous layer was extracted with EtOAc. The pH of the aqueous layer was adjusted to 14 and it was then extracted with EtOAc/THF (1:1). The combined organic layers were dried over anhydrous sodium sulfate and concentrated in vacuo to afford the title compound (1.92 g, 4.26 mmol, 88% yield) as a light brown oil. MS (ESI): 451.4 ([M+H]⁺).

### f) benzyl 4-[2-[3-[3-(3-amino-6-chloro-pyridazin-4-yl)-3,8-diazabicyclo[3.2.1]octan-8-yl]phenoxy]ethyl]piperazine-1-carboxylate

To a stirred suspension of benzyl 4-[2-[3-(3,8-diazabicyclo[3.2.1]octan-8-yl)phenoxy]ethyl]piperazine-1-carboxylate (1.92 g, 4.2 mmol, 1.0 eq) and 4-bromo-6-chloropyridazin-3-amine (1.15 g, 5.5 mmol, 1.3 eq) in DMSO (10.7 mL) at room temperature was added K₂CO₃ (3.53 g, 25.6 mmol, 6.0 eq). The reaction mixture was stirred for 15 h. The solvent was concentrated in vacuo. The residue was partitioned between EtOAc and 1 M aqueous hydrochloric acid. The aqueous layer was extracted with EtOAc, basified by addition of 40 mL 2 M aqueous sodium hydroxide solution and extracted with EtOAc/THF (1:1). The combined organic layers were dried over anhydrous sodium sulfate and concentrated in vacuo. The crude material was purified on silica column (DCM/MeOH 0-5%) to afford the title compound (1.92 g, 3.3 mmol, 78% yield) as a brown solid. MS (ESI): 578.4 (³⁵Cl[M+H]⁺).

### g) benzyl 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate

To a stirred suspension of benzyl 4-[2-[3-[3-(3-amino-6-chloro-pyridazin-4-yl)-3,8-diazabicyclo[3.2.1]octan-8-yl]phenoxy]ethyl]piperazine-1-carboxylate (1.925 g, 3.33 mmol, 1.0 eq) and (2-hydroxyphenyl)boronic acid (1.15 g, 8.32 mmol, 2.5 eq) in 1,4-dioxane (75.2 mL) and water (7.5 mL) at room temperature was added K₂CO₃ (1.61 g, 11.7 mmol, 3.5 eq). The reaction mixture was degassed with argon for 10 min. RuPhos Pd G3 (278 mg, 333 μmol, 0.1 eq) was added. The reaction mixture was heated at 90° C. for 2 h. The reaction mixture was then partitioned between EtOAc/THF (1:1) and water. The aqueous layer was extracted with EtOAc. The combined organic layers were washed with brine, dried over anhydrous sodium sulfate, and concentrated in vacuo. The crude material was purified on silica column (DCM/MeOH 0-10%) and on amine modified silica column (heptane/EtOAc 0-60%) to afford the title compound (1.02 g, 1.6 mmol, 48% yield) as a light yellow solid. MS (ESI): 636.5 ([M+H]⁺).

### h) 2-[6-amino-5-[8-[3-(2-piperazin-1-ylethoxy)phenyl]-3,8-diazabicyclo[3.2.1]octan-3-yl]pyridazin-3-yl]phenol

A stirred solution of benzyl 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate (0.98 g, 1.54 mmol, 1.0 eq) in methanol (100 mL) and THF (20 mL) at room temperature was degassed with argon for 10 min. 10% Pd on charcoal (328 mg, 308 μmol, 0.2 eq) was added. The reaction mixture was degassed with H₂ for 5 min. The reaction mixture was then stirred under a H₂ balloon at room temperature overnight. The reaction mixture was filtered and the filtrate was concentrated in vacuo to afford the title compound (697 mg, 1.39 mmol, 90% yield) as a yellow solid. MS (ESI): 500.3 ([M−H]⁻).

### i) tert-butyl 4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoate

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (170 mg, 339 μmol, 1.0 eq), 4-(tert-butoxy)-4-oxobutanoic acid (59 mg, 339 μmol, 1.0 eq) and DIPEA (131 mg, 178 μL, 1.02 mmol, 3.0 eq) in DMF (1 mL) at room temperature was added HATU (258 mg, 678 μmol, 2.0 eq). The reaction mixture was stirred for 4 h. The reaction mixture was partitioned between EtOAc/THF (1:1) and saturated aqueous bicarbonate solution. The aqueous layer was extracted with EtOAc. The combined organic layers were washed with brine, dried over anhydrous sodium sulfate, and concentrated in vacuo. The crude material was purified (DCM/MeOH 0-10%) on amine modified silica column (heptane/EtOAc 0-60%) to afford the title compound (79 mg, 120 μmol, 35% yield) as a white solid. MS (ESI): 658.4 ([M+H]⁺).

### j) 4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoic acid trihydrochloride

To a stirred solution of tert-butyl 4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoate (79 mg, 120 μmol, 1.0 eq) in 1,4-dioxane (0.5 mL) at room temperature was added 4 M hydrogen chloride solution in 1,4-dioxane (3.15 g, 3 mL, 12 mmol, 99.9 eq). The reaction mixture was stirred for 15 h. The solvent was concentrated in vacuo to afford the title compound (102 mg, 122 μmol, 102% yield) as a yellow solid, trihydrochloride salt. MS (ESI): 602.3 ([M+H]⁺).

### k) rac-4-[[1-[4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoyl]-4-piperidyl]oxy]-2-(2,6-dioxo-3-piperidyl)isoindoline-1,3-dione

To a stirred solution of 4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoic acid trihydrochloride (30 mg, 34.1 μmol, 1.0 eq), Ligase 7 (13.4 mg, 34.1 μmol, 1.0 eq, HCl salt) and DIPEA (35.3 mg, 47.7 μL, 273 μmol, 8.0 eq) in DMF (1 mL) at room temperature was added HATU (25.9 mg, 68.2 μmol, 2.0 eq). The reaction mixture was stirred at room temperature for 2 h. The crude material was purified by prep-HPLC to afford the title compound (12 mg, 10.3 μmol, 30% yield) as a yellow solid, bis-(2,2,2-trifluoroacetic acid)salt. MS (ESI): 939.4 ([M−H]⁻).

### Example 10

### rac-5-[[1-[4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoyl]-4-piperidyl]oxy]-2-(2,6-dioxo-3-piperidyl)isoindoline-1,3-dione

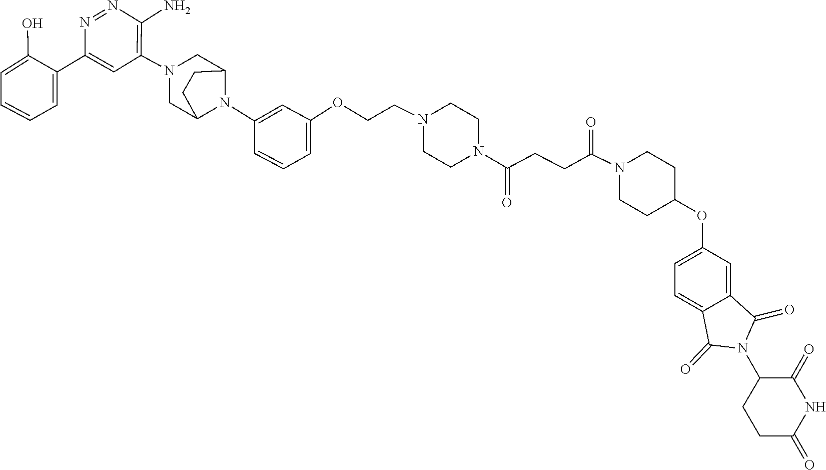 [SMILES: Nc1nnc(-c2ccccc2O)cc1N1CC2CCC(C1)N2c1cccc(OCCN2CCN(C(=O)CCC(=O)N3CCC(Oc4ccc5c(c4)C(=O)N(C4CCC(=O)NC4=O)C5=O)CC3)CC2)c1]

To a stirred solution of 4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoic acid trihydrochloride (30 mg, 34.1 μmol, 1.0 eq), Ligase 8 (13.4 mg, 34.1 μmol, 1.0 eq, HCl salt) and DIPEA (35.3 mg, 47.7 μL, 273 μmol, 8.0 eq) in DMF (1 mL) was added HATU (25.9 mg, 68.2 μmol, 2.0 eq). The reaction mixture was stirred for 2 h at room temperature. The crude material was purified by prep-HPLC to afford the title compound (12 mg, 10.3 μmol, 30% yield) as a yellow solid, bis-(2,2,2-trifluoroacetic acid)salt. MS (ESI): 939.4 ([M−H]⁻).

### Example 11

### rac-4-[[8-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-8-oxo-octyl]amino]-2-(2,6-dioxo-3-piperidyl)isoindoline-1,3-dione

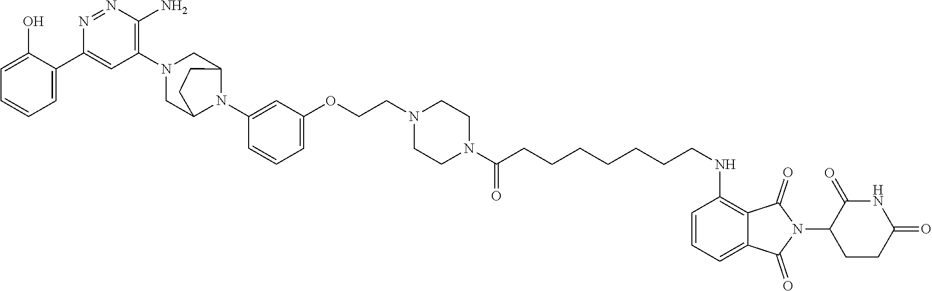 [SMILES: Nc1nnc(-c2ccccc2O)cc1N1CC2CCC(C1)N2c1cccc(OCCN2CCN(C(=O)CCCCCCCNc3cccc4c3C(=O)N(C3CCC(=O)NC3=O)C4=O)CC2)c1]

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (25 mg, 49.8 μmol, 1.0 eq), Ligase 9 (0.7 mg, 49.8 μmol, 1.0 eq) and DIPEA (19.3 mg, 26.1 μL, 150 μmol, 3.0 eq) in DMF (0.5 mL) at room temperature was added HATU (28.4 mg, 74.8 μmol, 1.5 eq). The reaction mixture was stirred at room temperature for 5 h. The crude material was purified by prep-HPLC to afford the title compound (23 mg, 20.4 μmol, 41% yield) as a yellow solid, bis-(2,2,2-trifluoroacetic acid)salt. MS (ESI): 897.4 ([M−H]⁻).

### Example 12

### rac-4-[[6-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-6-oxo-hexyl]amino]-2-(2,6-dioxo-3-piperidyl)isoindoline-1,3-dione

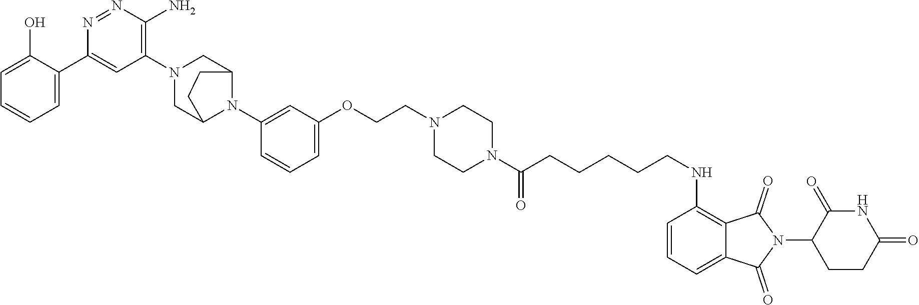 [SMILES: Nc1nnc(-c2ccccc2O)cc1N1CC2CCC(C1)N2c1cccc(OCCN2CCN(C(=O)CCCCCNc3cccc4c3C(=O)N(C3CCC(=O)NC3=O)C4=O)CC2)c1]

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (25 mg, 49.8 μmol, 1.0 eq), Ligase 10 (19.3 mg, 49.8 μmol, 1.0 eq) and DIPEA (16.1 mg, 21.8 μL, 125 μmol, 2.5 eq) in DMF (0.5 mL) at room temperature was added HATU (28.4 mg, 74.8 μmol, 1.5 eq). The reaction mixture was stirred at room temperature for 2 h. The crude material was purified by prep-HPLC to afford the title compound (26 mg, 23.7 μmol, 48% yield) as a yellow solid, bis-(2,2,2-trifluoroacetic acid)salt. MS (ESI): 869.4 ([M−H]⁻).

### Example 13

### rac-4-[4-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]-1-piperidyl]-2-(2,6-dioxo-3-piperidyl)isoindoline-1,3-dione

To a stirred solution of 2-[6-amino-5-[8-[3-(2-piperazin-1-ylethoxy)phenyl]-3,8-diazabicyclo[3.2.1]octan-3-yl]pyridazin-3-yl]phenol 28 mg, 55.8 μmol, 1.0 eq), Ligase 11 (21.5 mg, 55.8 μmol, 1.0 eq) and DIPEA (18 mg, 24.4 μL, 140 μmol, 2.5 eq) in DMF (0.5 mL) at room temperature was added HATU (31.8 mg, 83.7 μmol, 1.5 eq). The reaction mixture was stirred at room temperature for 2 h. The crude material was purified by prep-HPLC to afford the title compound (35 mg, 31.9 μmol, 57% yield) as a yellow solid, bis-(2,2,2-trifluoroacetic acid)salt. MS (ESI): 869.4 ([M+H]⁺).

### Example 14

### rac-5-[4-[3-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-3-oxo-propyl]-1-piperidyl]-2-(2,6-dioxo-3-piperidyl)isoindoline-1,3-dione

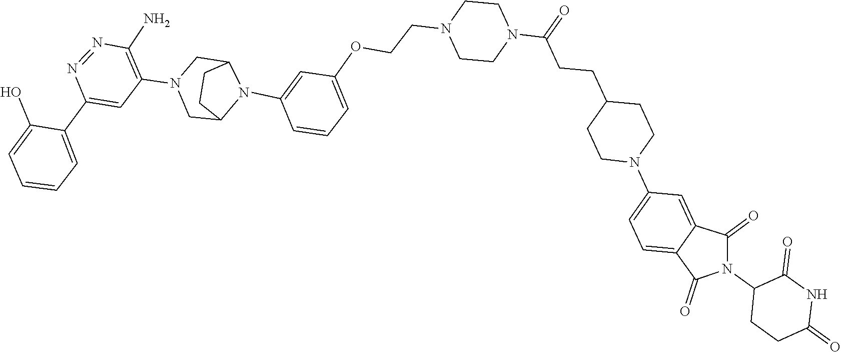 [SMILES: Nc1nnc(-c2ccccc2O)cc1N1CC2CCC(C1)N2c1cccc(OCCN2CCN(C(=O)CCC3CCN(c4ccc5c(c4)C(=O)N(C4CCC(=O)NC4=O)C5=O)CC3)CC2)c1]

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (28 mg, 55.8 μmol, 1.0 eq), Ligase 12 (23.1 mg, 55.8 μmol, 1.0 eq) and DIPEA (18 mg, 24.4 μL, 140 μmol, 2.5 eq) in DMF (0.5 mL) at room temperature was added HATU (31.8 mg, 83.7 umol, 1.5 eq). The reaction mixture was stirred at room temperature for 2 h. The crude material was purified by prep-HPLC to afford the title compound (23 mg, 20.4 μmol, 37% yield) as a yellow salt, bis-(2,2,2-trifluoroacetate) salt. MS (ESI): 897.4 ([M+H]⁺).

### Example 15

### rac-4-[4-[2-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-2-oxo-ethyl]-1-piperidyl]-2-(2,6-dioxo-3-piperidyl)isoindoline-1,3-dione

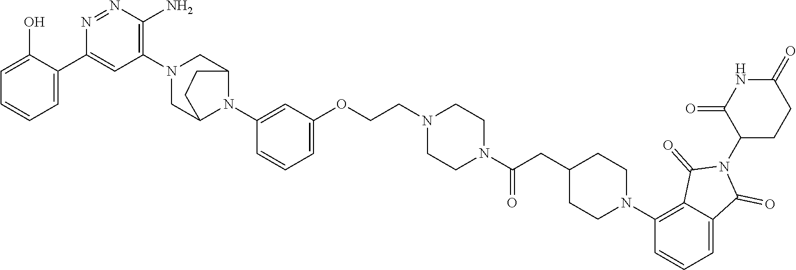 [SMILES: Nc1nnc(-c2ccccc2O)cc1N1CC2CCC(C1)N2c1cccc(OCCN2CCN(C(=O)CC3CCN(c4cccc5c4C(=O)N(C4CCC(=O)NC4=O)C5=O)CC3)CC2)c1]

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (28 mg, 55.8 μmol, 1.0 eq), Ligase 13 (22.3 mg, 55.8 μmol, 1.0 eq) and DIPEA (18 mg, 24.4 μL, 140 μmol, 2.5 eq) in DMF (0.5 mL) at room temperature was added HATU (31.8 mg, 83.7 μmol, 1.5 eq). The reaction mixture was stirred at room temperature for 2 h. The crude material was purified by prep-HPLC to afford the title compound (27 mg, 24.3 μmol, 44% yield) as a yellow solid, bis-(2,2,2-trifluoroacetate) salt. MS (ESI): 883.4 ([M+H]⁺).

### Example 16

### rac-4-[4-[3-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-3-oxo-propyl]-1-piperidyl]-2-(2,6-dioxo-3-piperidyl)isoindoline-1,3-dione

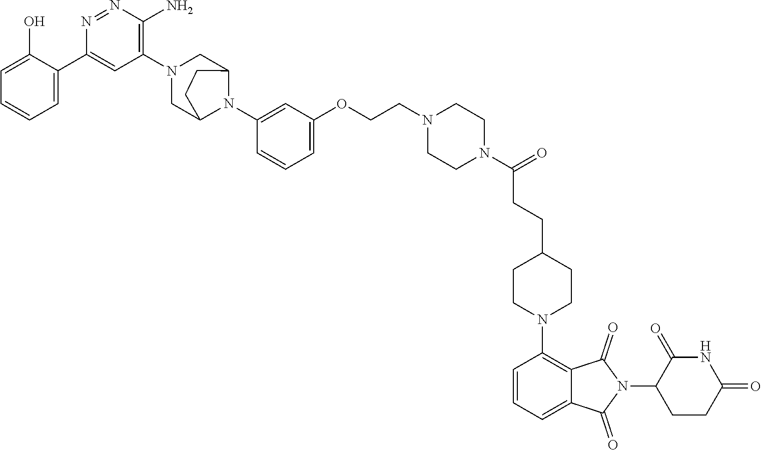 [SMILES: Nc1nnc(-c2ccccc2O)cc1N1CC2CCC(C1)N2c1cccc(OCCN2CCN(C(=O)CCC3CCN(c4cccc5c4C(=O)N(C4CCC(=O)NC4=O)C5=O)CC3)CC2)c1]

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (28 mg, 55.8 μmol, 1.0 eq), Ligase 2 (23.1 mg, 55.8 μmol, 1.0 eq) and DIPEA (18 mg, 24.4 μL, 140 μmol, 2.5 eq) in DMF (0.5 mL) at room temperature was added HATU (31.8 mg, 83.7 μmol, 1.5 eq). The reaction mixture was stirred at room temperature for 2 h. The crude material was purified by prep-HPLC and was lyophilised to afford the title compound (30 mg, 26.7 μmol, 48% yield) as a yellow solid, bis-(2,2,2-trifluoroacetate) salt. MS (ESI): 897.4 ([M+H]⁺).

### Example 17

### rac-N-[4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butyl]-2-[2-(2,6-dioxo-3-piperidyl)-1,3-dioxo-isoindolin-4-yl]oxy-acetamide

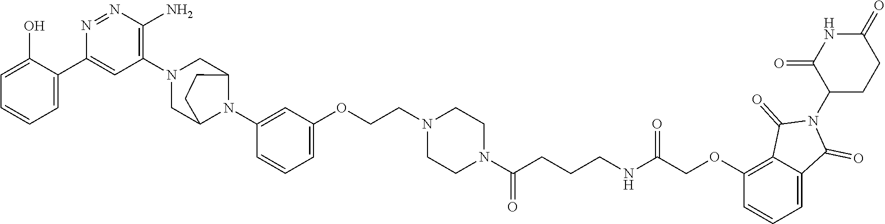 [SMILES: Nc1nnc(-c2ccccc2O)cc1N1CC2CCC(C1)N2c1cccc(OCCN2CCN(C(=O)CCCNC(=O)COc3cccc4c3C(=O)N(C3CCC(=O)NC3=O)C4=O)CC2)c1]

### a) tert-butyl N-[4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butyl]carbamate

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (120 mg, 239 μmol, 1.0 eq), DIPEA (92.8 mg, 125 μL, 718 μmol, 3.0 eq) and 4-((tert-butoxycarbonyl)amino)butanoic acid (48.6 mg, 239 μmol, 1.0 eq) in DMF (1 mL) at room temperature was added HATU (191 mg, 502 μmol, 2.1 eq). The reaction mixture was stirred for 4 h. The reaction mixture was partitioned between EtOAc/THF (1:1) and saturated aqueous bicarbonate solution. The aqueous layer was extracted with EtOAc. The combined organic layers were washed with brine, dried over anhydrous sodium sulfate, and concentrated in vacuo. The crude material was purified on silica column (DCM/MeOH 0-10%) to afford the title compound (82 mg, 95.5 μmol, 40% yield) as a yellow solid. MS (ESI): 687.4 ([M+H]⁺).

### b) 4-amino-1-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]butan-1-one

To a stirred solution of tert-butyl N-[4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butyl]carbamate (82 mg, 119 μmol, 1.0 eq) in 1,4-dioxane (1 mL) at room temperature was added 4 M hydrogen chloride solution in 1,4-dioxane (1.05 g, 1 mL, 4 mmol, 33.5 eq). The reaction mixture was stirred for 3 h. The precipitate was collected by filtration, washed with EtOAc, and dried in vacuo to afford the title compound (83 mg, 141 μmol, 118% yield) as a light yellow solid, trihydrochloride salt. MS (ESI): 587.3 ([M+H]⁺).

### c) rac-N-[4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butyl]-2-[2-(2,6-dioxo-3-piperidyl)-1,3-dioxo-isoindolin-4-yl]oxy-acetamide

To a stirred solution of 4-amino-1-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]butan-1-one trihydrochloride (41 mg, 58.9 μmol, 1.0 eq), Ligase 14 (19.6 mg, 58.9 μmol, 1.0 eq) and DIPEA (38.1 mg, 51.4 μL, 294 μmol, 5.0 eq) in DMF at room temperature was added HATU (44.8 mg, 118 μmol, 2.0 eq). The reaction mixture was stirred for 2 h. The crude material was purified by prep-HPLC to afford the title compound (10.5 mg, 9.3 μmol, 16% yield) as a yellow solid, bis-(2,2,2-trifluoroacetate) salt. MS (ESI): 901.4 ([M+H]⁺).

### Example 18

### rac-N-[2-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-2-oxo-ethyl]-1-[2-(2,6-dioxo-3-piperidyl)-1,3-dioxo-isoindolin-4-yl]piperidine-4-carboxamide

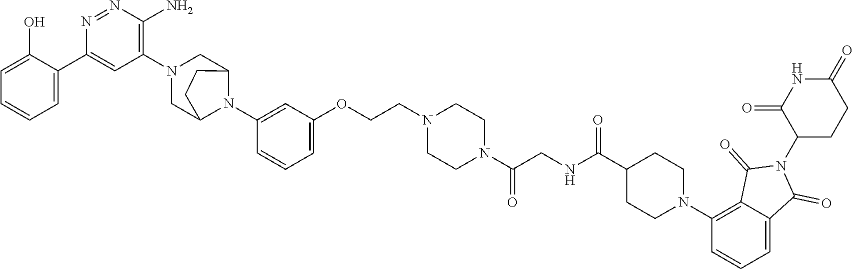 [SMILES: Nc1nnc(-c2ccccc2O)cc1N1CC2CCC(C1)N2c1cccc(OCCN2CCN(C(=O)CNC(=O)C3CCN(c4cccc5c4C(=O)N(C4CCC(=O)NC4=O)C5=O)CC3)CC2)c1]

### a) tert-butyl N-[2-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-2-oxo-ethyl]carbamate

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (120 mg, 239 μmol, 1.0 eq), (tert-butoxycarbonyl)glycine (41.9 mg, 239 μmol, 1.0 eq) and DIPEA (92.8 mg, 125 μL, 718 μmol, 3.0 eq) in DMF (1 mL) at room temperature was added HATU (182 mg, 478 μmol, 2.0 eq). The reaction mixture was stirred for 4 h. The reaction mixture was partitioned between EtOAc/THF (1:1) and saturated bicarbonate solution. The aqueous layer was extracted with EtOAc. The combined organic layers were washed with brine, dried over anhydrous sodium sulfate, and concentrated in vacuo. The crude material was purified on silica column (DCM/MeOH 0-10%), to afford the title compound (142 mg, 172 μmol, 72% yield) as a yellow solid. MS (ESI): 659.4 ([M+H]⁺).

### b) 2-amino-1-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]ethanone

To a stirred solution of tert-butyl N-[2-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-2-oxo-ethyl]carbamate (142 mg, 216 μmol, 1.0 eq) in 1,4-dioxane (1 mL) at room temperature was added 4 M hydrogen chloride solution in 1,4-dioxane (1.05 g, 1 mL, 4 mmol, 18.6 eq). The reaction mixture was stirred for 3 h. The precipitate was collected by filtration, washed with EtOAc, and dried in vacuo to afford the title compound (111 mg, 166 μmol, 77% yield) as a light yellow solid, trihydrochloride salt. MS (ESI): 559.3 ([M+H]⁺).

### c) rac-N-[2-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-2-oxo-ethyl]-1-[2-(2,6-dioxo-3-piperidyl)-1,3-dioxo-isoindolin-4-yl]piperidine-4-carboxamide

To a stirred solution of 2-amino-1-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]ethanone trihydrochloride (40 mg, 59.9 μmol, 1.0 eq), Ligase 11 (23.1 mg, 59.9 μmol, 1.0 eq) and DIPEA (38.7 mg, 52.3 μL, 299 μmol, 5.0 eq) in DMF (0.7 mL) at room temperature was added HATU (50.1 mg, 132 μmol, 2.2 eq). The reaction mixture was stirred for 2 h. The crude material was purified by prep-HPLC to afford the title compound (10.4 mg, 9.01 μmol, 15% yield) as a yellow solid, bis-(2,2,2-trifluoroacetate) salt. MS (ESI): 926.4 ([M+H]⁺).

### Example 19

### rac-N-[2-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-2-oxo-ethyl]-1-[2-(2,6-dioxo-3-piperidyl)-1,3-dioxo-isoindolin-5-yl]piperidine-4-carboxamide

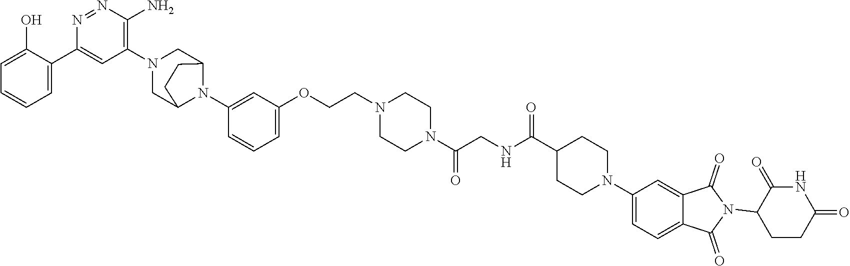 [SMILES: Nc1nnc(-c2ccccc2O)cc1N1CC2CCC(C1)N2c1cccc(OCCN2CCN(C(=O)CNC(=O)C3CCN(c4ccc5c(c4)C(=O)N(C4CCC(=O)NC4=O)C5=O)CC3)CC2)c1]

To a stirred solution of 2-amino-1-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]ethanone trihydrochloride (40 mg, 59.9 μmol, 1.0 eq), Ligase 15 (23.1 mg, 59.9 μmol, 1.0 eq) and DIPEA (38.7 mg, 52.3 μL, 299 μmol, 5.0 eq) in DMF (0.7 mL) at room temperature was added HATU (50.1 mg, 132 μmol, 2.2 eq). The reaction mixture was stirred for 2 h. The crude material was purified by prep-HPLC to afford the title compound (8.5 mg, 7.37 μmol, 12% yield) as a yellow solid, bis-(2,2,2-trifluoroacetic acid)salt. MS (ESI): 926.4 ([M+H]⁺).

### Example 20

### rac-4-[[1-[4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoyl]-4-piperidyl]amino]-2-(2,6-dioxo-3-piperidyl)isoindoline-1,3-dione

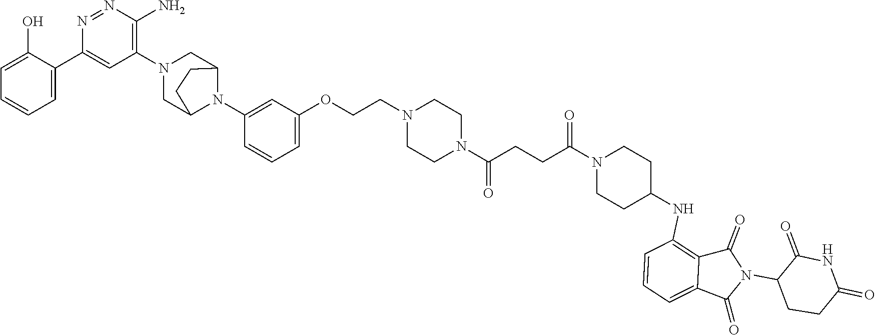 [SMILES: Nc1nnc(-c2ccccc2O)cc1N1CC2CCC(C1)N2c1cccc(OCCN2CCN(C(=O)CCC(=O)N3CCC(Nc4cccc5c4C(=O)N(C4CCC(=O)NC4=O)C5=O)CC3)CC2)c1]

### a) tert-butyl 4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoate

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (170 mg, 339 μmol, 1.0 eq), 4-(tert-butoxy)-4-oxobutanoic acid (59 mg, 339 μmol, 1.0 eq) and DIPEA (131 mg, 178 μL, 1.02 mmol, 3.0 eq) in DMF (1 mL) at room temperature was added HATU (258 mg, 678 μmol, 2.0 eq). The reaction mixture was stirred for 4 h. The reaction mixture was partitioned between EtOAc/THF (1:1) and saturated aqueous bicarbonate solution. The aqueous layer was extracted with EtOAc. The combined organic layers were washed with brine, dried over anhydrous sodium sulfate, and concentrated in vacuo. The crude material was purified on silica column (DCM/MeOH 0-10%) followed amine modified silica column (heptane/EtOAc 0-40%) to afford the title compound (79 mg, 120 μmol, 35% yield) as a white solid. MS (ESI): 658.4 ([M+H]⁺).

### b) 4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoic acid trihydrochloride

To a stirred solution of tert-butyl 4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoate (79 mg, 120 μmol, 1.0 eq) in 1,4-dioxane (0.5 mL) at room temperature was added 4 M hydrogen chloride solution in 1,4-dioxane (3.15 g, 3 mL, 12 mmol, 99.9 eq). The reaction mixture was stirred for 15 h. The solvent was concentrated in vacuo to afford the title compound (102 mg, 122 μmol, 102% yield) as a yellow solid, trihydrochloride salt. MS (ESI): 602.3 ([M+H]⁺).

### c) rac-4-[[1-[4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoyl]-4-piperidyl]amino]-2-(2,6-dioxo-3-piperidyl)isoindoline-1,3-dione

To a stirred solution of 4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoic acid trihydrochloride (30 mg, 42.2 μmol, 1.0 eq), DIPEA (27.3 mg, 36.8 μL, 211 μmol, 5.0 eq) and Ligase 16 (16.6 mg, 42.2 μmol, 1.0 eq, HCl salt) in DMF (0.7 mL) at room temperature was added HATU (35.3 mg, 92.8 μmol, 2.2 eq). The reaction mixture was stirred for 3 h. The crude material was purified by prep-HPLC to afford the title compound (13.8 mg, 11.8 μmol, 28% yield) as a yellow solid, bis-(2,2,2-trifluoroacetic acid)salt. MS (ESI): 940.4 ([M+H]⁺).

### Example 21

### rac-4-[[1-[4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoyl]-4-piperidyl]-methyl-amino]-2-(2,6-dioxo-3-piperidyl)isoindoline-1,3-dione

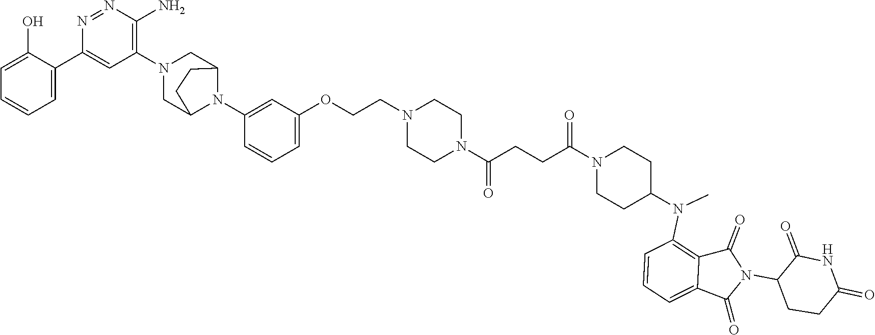 [SMILES: CN(c1cccc2c1C(=O)N(C1CCC(=O)NC1=O)C2=O)C1CCN(C(=O)CCC(=O)N2CCN(CCOc3cccc(N4C5CCC4CN(c4cc(-c6ccccc6O)nnc4N)C5)c3)CC2)CC1]

To a stirred solution of 4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoic acid trihydrochloride (30 mg, 42.2 μmol, 1.0 eq), DIPEA (27.3 mg, 36.8 μL, 211 μmol, 5.0 eq) and Ligase 17 (17.2 mg, 42.2 μmol, 1.0 eq, HCl salt) in DMF (0.7 mL) at room temperature was added HATU (35.3 mg, 92.8 μmol, 2.2 eq). The reaction mixture was stirred for 3 h. The crude material was purified by prep-HPLC to afford the title compound (17.5 mg, 14.8 μmol, 35% yield) as a yellow solid, bis-(2,2,2-trifluoroacetate) salt. MS (ESI). 954.5 ([M+H]⁺).

### Example 22

### rac-4-[[4-[4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoyl]piperazin-1-yl]methyl]-2-(2,6-dioxo-3-piperidyl)isoindoline-1,3-dione

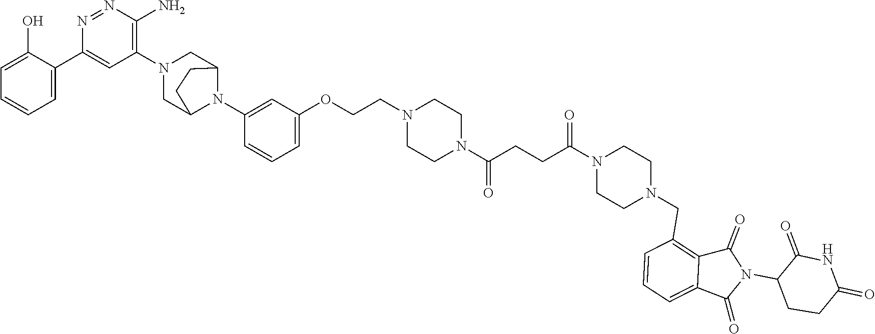 [SMILES: Nc1nnc(-c2ccccc2O)cc1N1CC2CCC(C1)N2c1cccc(OCCN2CCN(C(=O)CCC(=O)N3CCN(Cc4cccc5c4C(=O)N(C4CCC(=O)NC4=O)C5=O)CC3)CC2)c1]

To a stirred solution of 4-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-4-oxo-butanoic acid trihydrochloride (30 mg, 42.2 μmol, 1.0 eq), DIPEA (27.3 mg, 36.8 μL, 211 μmol, 5.0 eq) and Ligase 18 (16.6 mg, 42.2 μmol, 1.0 eq) in DMF (0.7 mL) at room temperature was added HATU (35.3 mg, 92.8 μmol, 2.2 eq). The reaction mixture was stirred for 3 h. The crude material was purified by prep-HPLC to afford the title compound (13.2 mg, 11.3 μmol, 27% yield) as an off-white solid, bis-(2,2,2-trifluoroacetate) salt. MS (ESI): 940.4 ([M+H]⁺).

### Example 23

### 4-[[1-[12-[4-[2-[4-[3-[3-amino-6-(2-hydroxyphenyl)pyridazin-4-yl]oxy-1-piperidyl]phenoxy]ethyl]piperazin-1-yl]-12-oxo-dodecyl]triazol-4-yl]methylamino]-2-(2,6-dioxo-3-piperidyl)isoindoline-1,3-dione

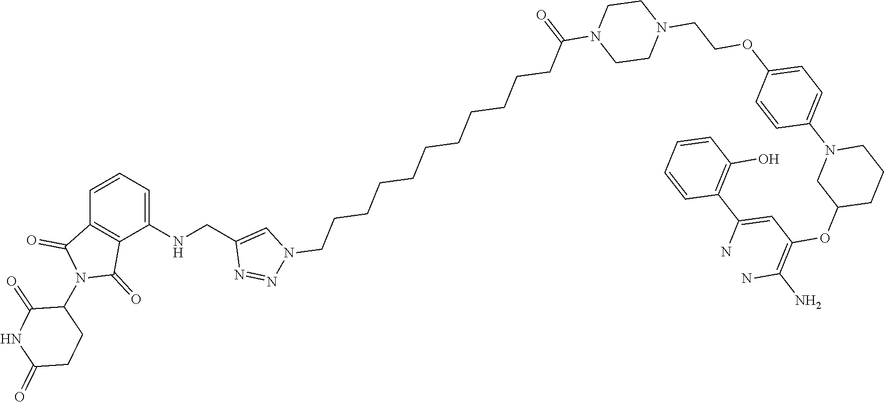 [SMILES: NC(N)=C(/C=C(\N)c1ccccc1O)OC1CCCN(c2ccc(OCCN3CCN(C(=O)CCCCCCCCCCCn4cc(CNc5cccc6c5C(=O)N(C5CCC(=O)NC5=O)C6=O)nn4)CC3)cc2)C1]

### a) tert-Butyl 4-(2-(4-iodophenoxy)ethyl)piperazine-1-carboxylate

A sealed tube was charged with 4-iodophenol (6 g, 27.2 mmol), tert-butyl 4-(2-bromoethyl)piperazine-1-carboxylate (8 g, 27.2 mmol), potassium carbonate (7.5 g, 54.2 mmol) and acetone (60 mL). The reaction mixture was heated to 60° C. for 16 h. The reaction mixture was cooled to room temperature, filtered on celite bed and washed with EtOAc and concentrated under reduced pressure. The crude residue was purified on silica column (heptane/EtOAc 0-50%) to afford the title compound (9.5 g, 21.9 mmol, 80% yield) as an off white solid. MS (ESI): 433.0 ([M+H]⁺).

### b) tert-butyl 4-(2-(4-(3-hydroxypiperidin-1-yl)phenoxy)ethyl)piperazine-1-carboxylate

A sealed tube was charged with tert-butyl 4-[2-(4-iodophenoxy)ethyl]piperazine-1-carboxylate (9.5 g, 21.9 mmol), piperidin-3-ol (5.56 g, 54.9 mmol), potassium phosphate tribasic anhydrous (4 g, 65.9 mmol) and L-proline (1.27 g, 10.9 mmol, 930 μL) and DMF (95 mL). The reaction mixture was purged with nitrogen for 15 min and was added copper (I) iodide (2.1 g, 10.9 mmol), and the reaction mixture was heated to 100° C. for 16 h. The reaction was cooled to room temperature passed through celite bed, washed with EtOAc and concentrated under reduced pressure. The crude residue was purified on silica column (PE/EtOAc 0-90%) as eluent to afford the title compound (4.5 g, 11.10 mmol, 50% yield) as a light brown semi solid. MS (ESI): 406.0 ([M+H]⁺).

### c) tert-butyl 4-(2-(4-(3-((3-amino-6-chloropyridazin-4-yl)oxy)piperidin-1-yl)phenoxy)ethyl)piperazine-1-carboxylate

To a solution of tert-butyl 4-[2-[4-(3-hydroxy-1-piperidyl)phenoxy]ethyl]piperazine-1-carboxylate (4.5 g, 11.1 mmol) in DMF (60 mL) was added sodium hydride, 60% dispersion in mineral oil (765 mg, 33.3 mmol) at 0° C. The reaction mixture was warmed to 60° C. and stirred for 1 h. The reaction mixture was cooled to 0° C. and was added 4-bromo-6-chloro-pyridazin-3-amine (5.78 g, 27.7 mmol) in DMF (30 mL) dropwise. The reaction mixture was heated to 90° C. for 3 h. The reaction mixture was quenched with saturated ammonium chloride solution, diluted with water, extracted with EtOAc. The organic layer was washed with water, brine solution, dried over sodium sulfate, filtered and concentrated under reduced pressure. The crude residue was purified on silica column (PE/EtOAc 50-90%) as eluent to afford the title compound (1.7 g, 2.92 mmol, 26% yield) as a light brown semi solid. MS (ESI): 533.2 ([M+H]⁺).

### d) tert-butyl 4-(2-(4-(3-((3-amino-6-(2-hydroxyphenyl)pyridazin-4-yl)oxy)piperidin-1-yl)phenoxy)ethyl)piperazine-1-carboxylate

In a sealed tube a mixture of tert-butyl 4-[2-[4-[3-(3-amino-6-chloro-pyridazin-4-yl)oxy-1-piperidyl]phenoxy]ethyl]piperazine-1-carboxylate (1.7 g, 3.19 mmol), (2-hydroxyphenyl)boronic acid (484 mg, 3.51 mmol) and potassium carbonate (1.32 g, 9.57 mmol) was diluted in 1,4-dioxane (35 mL) and water (7 mL) and the reaction mixture was degassed with nitrogen for 10 min. To the resulting mixture was added bis(diphenylphosphino)ferrocene]dichloropalladium(II), complex with DCM (260 mg, 318 umol), again degassed for 10 minutes and heated to 100° C. for 16 h. The reaction was cooled to room temperature passed through celite bed, washed with EtOAc and concentrated under reduced pressure. The crude residue was purified on silica column (DCM/MeOH 0-3%) to afford the title compound (1.1 g, 1.64 mmol, 51.38% yield) as a light brown semi solid. MS (ESI): 590.0 ([M+H]⁺).

### e) 2-(6-amino-5-((1-(4-(2-(piperazin-1-yl)ethoxy)phenyl)piperidin-3-yl)oxy)pyridazin-3-yl)phenol

To a solution of tert-butyl 4-[2-[4-[3-[3-amino-6-(2-hydroxyphenyl)pyridazin-4-yl]oxy-1-piperidyl]phenoxy]ethyl]piperazine-1-carboxylate (460 mg, 778 μmol) in DCM (6 mL) was added trifluoroacetic acid (444 mg, 3.89 mmol, 299 μL) at 0° C. The reaction mixture was warmed to room temperature and stirred for 3 h. The reaction mixture was concentrated under reduced pressure, distilled with chloroform (2×20 mL) to afford the title compound (375 mg, 764 μmol, 98% yield) as a light brown semi solid. MS (ESI): 491.2 ([M+H]⁺).

### f) 4-[[1-[12-[4-[2-[4-[3-[3-amino-6-(2-hydroxyphenyl)pyridazin-4-yl]oxy-1-piperidyl]phenoxy]ethyl]piperazin-1-yl]-12-oxo-dodecyl]triazol-4-yl]methylamino]-2-(2,6-dioxo-3-piperidyl)isoindoline-1,3-dione

A screw cap vial (8 mL) was charged with 2-[6-amino-5-[[1-[4-(2-piperazin-1-ylethoxy)phenyl]-3-piperidyl]oxy]pyridazin-3-yl]phenol (25 mg, 0.041 mmol), Ligase 4 (0.041 mmol), DMF (0.5 mL) and to this reaction mixture was added DIPEA (28 mg, 0.038 mL, 0.22 mmol) followed by HATU (24 mg, 0.062 mmol). The reaction mixture was stirred at room temperature for 16 h, diluted with water and extracted with EtOAc. The volatiles were removed. The crude residue was purified by prep-HPLC to afford the title compound (13.2 mg, 12.2 μmol, 29% yield) as an off-white solid, trifluoroacetic acid salt. MS (ESI): 1026.1 ([M+H]⁺).

### Example 24

### 4-[[1-[15-[4-[2-[4-[3-[3-amino-6-(2-hydroxyphenyl)pyridazin-4-yl]oxy-1-piperidyl]phenoxy]ethyl]piperazin-1-yl]-15-oxo-pentadecyl]triazol-4-yl]methylamino]-2-(2,6-dioxo-3-piperidyl)isoindoline-1,3-dione

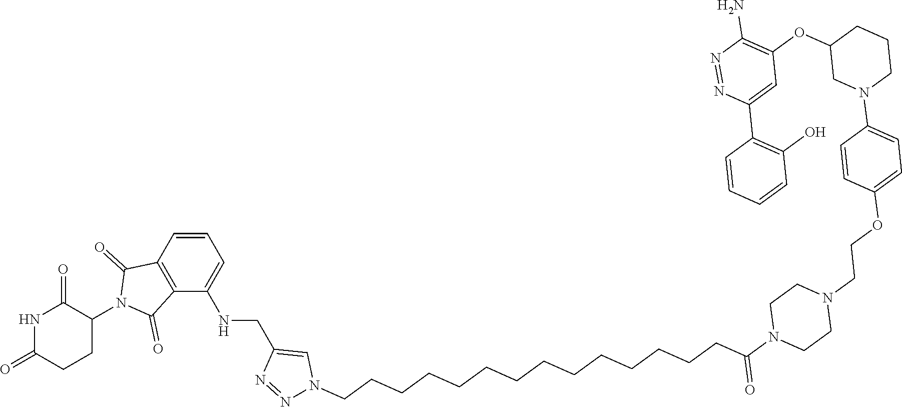 [SMILES: Nc1nnc(-c2ccccc2O)cc1OC1CCCN(c2ccc(OCCN3CCN(C(=O)CCCCCCCCCCCCCCn4cc(CNc5cccc6c5C(=O)N(C5CCC(=O)NC5=O)C6=O)nn4)CC3)cc2)C1]

The title compound was prepared in analogy to example 23 step f using Ligase 6 as an off-white solid (22.0 mg, 20 μmol, 48% yield), trifluoro acetic acid salt. MS (ESI): 1068.1 ([M+H]+).

### Example 25

### 3-[4-[4-[14-[4-[2-[4-[3-[3-amino-6-(2-hydroxyphenyl)pyridazin-4-yl]oxy-1-piperidyl]phenoxy]ethyl]piperazin-1-yl]-14-oxo-tetradecanoyl]piperazin-1-yl]anilino]piperidine-2,6-dione

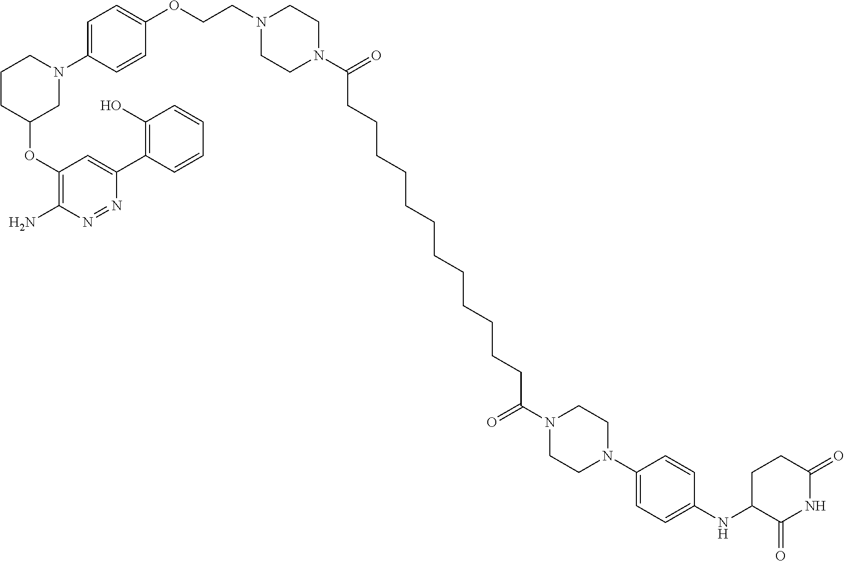 [SMILES: Nc1nnc(-c2ccccc2O)cc1OC1CCCN(c2ccc(OCCN3CCN(C(=O)CCCCCCCCCCCCC(=O)N4CCN(c5ccc(NC6CCC(=O)NC6=O)cc5)CC4)CC3)cc2)C1]

The title compound was prepared in analogy to example 23 step f using Ligase 19 as an off-white solid (21.0 mg, 18.53 μmol, 44% yield), trifluoro acetic acid salt. MS (ESI): 1002.1 ([M+H]⁺).

### Example 26

### 5-[4-[9-[4-[4-[3-[3-amino-6-(2-hydroxyphenyl)pyridazin-4-yl]oxy-1-piperidyl]phenyl]piperazin-1-yl]-9-oxo-nonyl]piperazin-1-yl]-2-(2,6-dioxo-3-piperidyl)isoindoline-1,3-dione

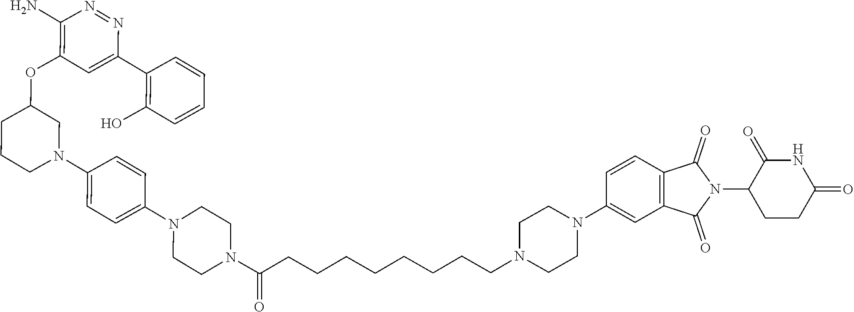 [SMILES: Nc1nnc(-c2ccccc2O)cc1OC1CCCN(c2ccc(N3CCN(C(=O)CCCCCCCCN4CCN(c5ccc6c(c5)C(=O)N(C5CCC(=O)NC5=O)C6=O)CC4)CC3)cc2)C1]

To a solution 2-[6-amino-5-[[1-(4-piperazin-1-ylphenyl)-3-piperidyl]oxy]pyridazin-3-yl]phenol (40 mg, 0.089 mmol) and Ligase 20 (44 mg, 0.089 mmol) in DMF (0.4 mL) was added HATU (51 mg, 0.134 mmol) followed by DIPEA (57 mg, 0.447 mmol, 0.078 mL) and the reaction mixture was stirred at room temperature for 16 h. The reaction mixture was diluted with water and extracted with EtOAc. The volatiles were removed. The crude residue was purified by prep-HPLC to afford the title compound (4.53 mg, 4.89 μmol, 5.4% yield) as an off-white solid, trifluoro acetic acid salt. MS (ESI): 927.4 ([M+H]⁺).

### Example 27

### 4-[[1-[9-[4-[2-[4-[3-[3-amino-6-(2-hydroxyphenyl)pyridazin-4-yl]oxy-1-piperidyl]phenoxy]ethyl]piperazin-1-yl]-9-oxo-nonyl]triazol-4-yl]methylamino]-2-(2,6-dioxo-3-piperidyl)isoindoline-1,3-dione

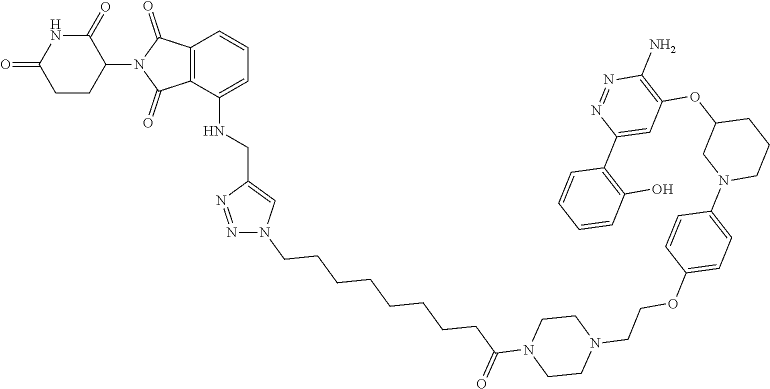 [SMILES: Nc1nnc(-c2ccccc2O)cc1OC1CCCN(c2ccc(OCCN3CCN(C(=O)CCCCCCCCn4cc(CNc5cccc6c5C(=O)N(C5CCC(=O)NC5=O)C6=O)nn4)CC3)cc2)C1]

The title compound (1.80 mg, 1.69 μmol, 4% yield) was prepared in analogy to example 23 step f using Ligase 3 as an off-white solid, trifluoro acetic acid salt. MS (ESI): 984.0 ([M+H]⁺).

### Example 28

### 4-[[1-[10-[4-[4-[3-[3-amino-6-(2-hydroxyphenyl)pyridazin-4-yl]oxy-1-piperidyl]phenyl]piperazin-1-yl]-10-oxo-decyl]triazol-4-yl]methylamino]-2-(2,6-dioxo-3-piperidyl)isoindoline-1,3-dione

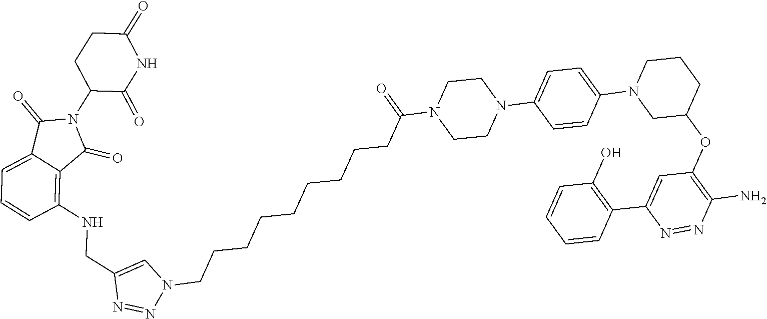 [SMILES: Nc1nnc(-c2ccccc2O)cc1OC1CCCN(c2ccc(N3CCN(C(=O)CCCCCCCCCn4cc(CNc5cccc6c5C(=O)N(C5CCC(=O)NC5=O)C6=O)nn4)CC3)cc2)C1]

To a solution of 2-[6-amino-5-[[1-(4-piperazin-1-ylphenyl)-3-piperidyl]oxy]pyridazin-3-yl]phenol (40 mg, 71.4 μmol, TFA salt) and Ligase 21 (37 mg, 71.48 μmol) in DMF (1 mL) was added HATU (40 mg, 107.23 μmol) followed by DIPEA (46 mg, 357.42 umol, 62 μL) and the reaction mixture was stirred at room temperature for 16 h. The reaction mixture was diluted with water and extracted with EtOAc. The volatiles were removed. The crude residue was purified by prep-HPLC to afford the title compound (18.8 mg, 18 umol, 26% yield) as a light green solid. MS (ESI): 954.1 ([M+H]1).

### Example 29

### 4-[[1-[11-[4-[4-[3-[3-amino-6-(2-hydroxyphenyl)pyridazin-4-yl]oxy-1-piperidyl]phenyl]piperazin-1-yl]-11-oxo-undecyl]triazol-4-yl]methylamino]-2-(2,6-dioxo-3-piperidyl)isoindoline-1,3-dione

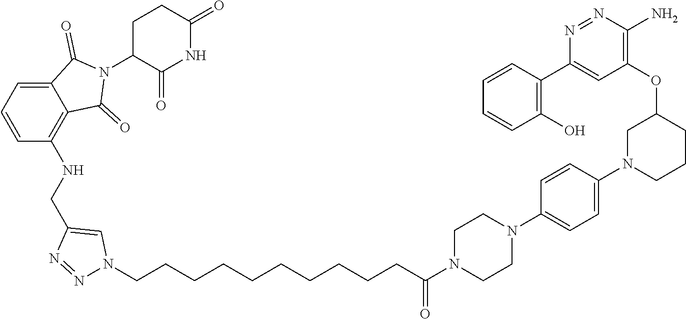 [SMILES: Nc1nnc(-c2ccccc2O)cc1OC1CCCN(c2ccc(N3CCN(C(=O)CCCCCCCCCCn4cc(CNc5cccc6c5C(=O)N(C5CCC(=O)NC5=O)C6=O)nn4)CC3)cc2)C1]

To a solution of 2-[6-amino-5-[[1-(4-piperazin-1-ylphenyl)-3-piperidyl]oxy]pyridazin-3-yl]phenol (40 mg, 71.48 μmol, TFA salt) and Ligase 22 (38 mg, 71.48 μmol) in DMF (1 mL) was added HATU (40 mg, 107.23 μmol) followed by DIPEA (46 mg, 357.42 μmol, 62.26 μL). The reaction mixture was stirred at room temperature for 16 h. The reaction mixture was diluted with water and extracted with EtOAc. The volatiles were removed. The crude residue was purified by prep-HPLC to afford the title compound (6.9 mg, 7.0 μmol, 9% yield) as a light green solid. MS (ESI): 968.1 ([M+H]⁺).

### Example 30

### 4-[[7-[4-[[3-[1-[3-amino-6-(2-hydroxyphenyl)pyridazin-4-yl]azetidin-3-yl]oxyphenyl]methyl]piperazin-1-yl]-7-oxo-heptyl]amino]-2-(2,6-dioxo-3-piperidyl)isoindoline-1,3-dione

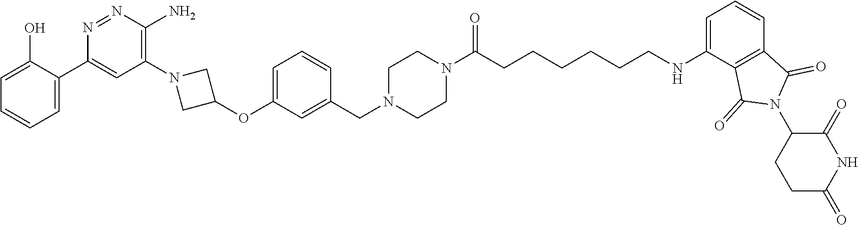 [SMILES: Nc1nnc(-c2ccccc2O)cc1N1CC(Oc2cccc(CN3CCN(C(=O)CCCCCCNc4cccc5c4C(=O)N(C4CCC(=O)NC4=O)C5=O)CC3)c2)C1]

### a) tert-butyl 3-(3-formylphenoxy)azetidine-1-carboxylate

A mixture of 1-boc-3-iodoazetidine (6.03 g, 21.29 mmol, 1.3 eq), 3-hydroxybenzaldehyde (2 g, 16.38 mmol, 1.0 eq) and cesium carbonate (9.6 g, 29.48 mmol, 1.8 eq) in DMF (15 mL) was stirred at 150° C. for 1 h under the microwave condition. The reaction mixture was poured into water, extracted with EtOAc, dried over Na₂SO₄ and concentrated in vacuum to afford the title compound (2 g, 7.2 mmol, 44% yield) as a yellow oil.

### b) benzyl 4-(3-((1-(tert-butoxycarbonyl)azetidin-3-yl)oxy)benzyl)piperazine-1-carboxylate

A mixture of 1-Cbz-piperazine (1.9 g, 8.65 mmol, 1.2 eq), tert-butyl 3-(3-formylphenoxy)azetidine-1-carboxylate (2 g, 7.21 mmol, 1.0 eq) and acetic acid (0.5 mL, 7.21 mmol, 1.0 eq) in DME (50 mL) was stirred at 25° C. for 1 h. Sodium cyanoborohydride (906 mg, 14.4 mmol, 2.0 eq) was added, the reaction mixture was stirred at 25° C. for 12 h. The reaction mixture was concentrated in vacuum and purified by prep-HPLC to afford the title compound (2 g, 4.1 mmol, 57% yield) as a yellow oil. MS (ESI): 482.4 ([M+H]⁺).

### c) benzyl 4-(3-(azetidin-3-yloxy)benzyl)piperazine-1-carboxylate

A mixture of benzyl 4-[[3-(1-tert-butoxycarbonylazetidin-3-yl)oxyphenyl]methyl]piperazine-1-carboxylate (2 g, 4.15 mmol, 1.0 eq) in trifluoroacetic acid (5.0 mL, 44.85 mmol, 11.0 eq) and DCM (20 mL) was stirred at 25° C. for 2 h. The reaction mixture was concentrated in vacuum to afford the title compound (1.5 g, 3.9 mmol, 94% yield) as a yellow oil. MS (ESI): 382.3 ([M+H]⁺).

### d) benzyl 4-(3-((1-(3-amino-6-chloropyridazin-4-yl)azetidin-3-yl)oxy)benzyl)piperazine-1-carboxylate

A mixture of benzyl 4-[[3-(azetidin-3-yloxy)phenyl]methyl]piperazine-1-carboxylate (1.5 g, 3.9 mmol, 1.0 eq), 4-bromo-6-chloro-pyridazin-3-amine (820 mg, 3.9 mmol, 1.0 eq) and triethylamine (1.2 g, 11.8 mmol, 3.0 eq) in DMF (10 mL) was stirred at 100° C. for 12 h. The reaction mixture was purified by prep-HPLC to afford the title compound (1.5 g, 2.95 mmol, 54% yield) as a yellow oil. MS (ESI): 509.3 ([M+H]⁺).

### e) benzyl 4-(3-((1-(3-amino-6-(2-hydroxyphenyl)pyridazin-4-yl)azetidin-3-yl)oxy)benzyl) piperazine-1-carboxylate

A mixture of RuPhos-Pd-G3 (30 mg, 0.020 mmol, 0.05 eq), potassium carbonate (285 mg, 2.06 mmol, 3.5 eq), benzyl 4-[[3-[1-(3-amino-6-chloro-pyridazin-4-yl)azetidin-3-yl]oxyphenyl]methyl]piperazine-1-carboxylate (300 mg, 0.59 mmol, 1.0 eq) and 2-hydroxyphenylboronic acid (203 mg, 1.47 mmol, 2.5 eq) in 1,4-dioxane (18 mL) and water (1.8 mL) was stirred at 90° C. for 2 h under N₂ atmosphere. The reaction mixture was purified by prep-HPLC to afford the title compound (300 mg, 0.53 mmol, 89% yield) as a yellow oil. MS (ESI): 567.4 ([M+H]⁺).

### f) 2-(6-amino-5-(3-(3-(piperazin-1-ylmethyl)phenoxy)azetidin-1-yl)pyridazin-3-yl)phenol

A mixture of benzyl 4-[[3-[1-[3-amino-6-(2-hydroxyphenyl)pyridazin-4-yl]azetidin-3-yl]oxyphenyl]methyl]piperazine-1-carboxylate (300 mg, 0.53 mmol) and 10% palladium on active carbon (100 mg) in methanol (10 mL) was stirred at 25° C. for 12 h under H₂ atmosphere (15 psi). The reaction mixture was filtered and the filtrate was purified by prep-HPLC to afford the title compound (150 mg, 0.35 mmol, 65% yield) as a white solid. MS (ESI): 433.3 ([M+H]⁺).

### g) 4-[[7-[4-[[3-[1-[3-amino-6-(2-hydroxyphenyl)pyridazin-4-yl]azetidin-3-yl]oxyphenyl]methyl]piperazin-1-yl]-7-oxo-heptyl]amino]-2-(2,6-dioxo-3-piperidyl)isoindoline-1,3-dione

A mixture of Ligase 1 (33 mg, 0.080 mmol, 1.1 eq), Et₃N (0.5 mL, 0.210 mmol, 3.0 eq), T₃P (36 mg, 0.080 mmol, 1.1 eq) and 2-[6-amino-5-[3-[3-(piperazin-1-ylmethyl)phenoxy]azetidin-1-yl]pyridazin-3-yl]phenol (30 mg, 0.070 mmol, 1.0 eq) in DMF (3 mL) was stirred at 25° C. for 1 h. The reaction mixture was purified by prep-HPLC to afford the title compound (3.2 mg, 5.5% yield) as a yellow solid. MS (ESI): 816.6 ([M+H]⁺).

### Example 31

### rac-5-[4-[6-[2-[4-[3-[3-[3-amino-6-(2-hydroxyphenyl)pyridazin-4-yl]-3,8-diazabicyclo[3.2.1]octan-8-yl]phenoxy]cyclohexyl]acetyl]-2,6-diazaspiro[3.3]heptane-2-carbonyl]-1-piperidyl]-2-(2,6-dioxo-3-piperidyl)isoindoline-1,3-dione

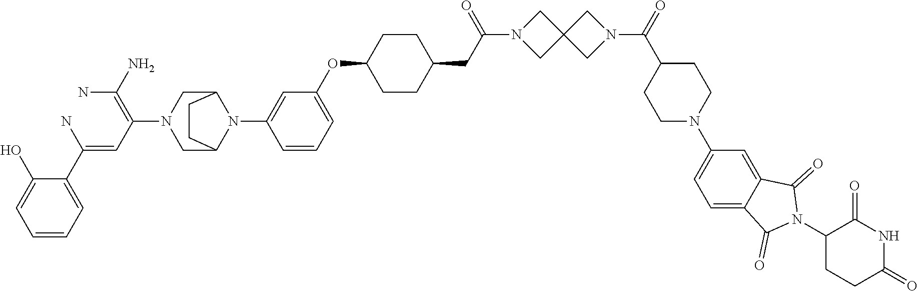 [SMILES: NC(N)=C(/C=C(\N)c1ccccc1O)N1CC2CCC(C1)N2c1cccc(O[C@H]2CC[C@@H](CC(=O)N3CC4(C3)CN(C(=O)C3CCN(c5ccc6c(c5)C(=O)N(C5CCC(=O)NC5=O)C6=O)CC3)C4)CC2)c1]

### a) tert-butyl 8-(3-benzyloxyphenyl)-3,8-diazabicyclo[3.2.1]octane-3-carboxylate

To a stirred suspension of 1-(benzyloxy)-3-bromobenzene (3 g, 11.4 mmol, 1.0 eq) and tert-butyl 3,8-diazabicyclo[3.2.1]octane-3-carboxylate (2.54 g, 12 mmol, 1.05 eq) in t-BuOH (18.5 mL) at room temperature was added K₂CO₃ (3.15 g, 22.8 mmol, 2.0 eq). The reaction was degassed with argon for 5 min. RuPhos Pd G3 (954 mg, 1.14 mmol, 0.1 eq) was added. The reaction mixture was stirred at 120° C. overnight. The reaction mixture was poured into EtOAc/THF (2:1) and washed with brine. The organic layer was dried over Na₂SO₄ and concentrated in vacuo. The crude material was purified on silica column (heptane/EtOAc 0-30%) to afford the title compound (1.94 g, 4.9 mmol, 43% yield) as a yellow solid. MS (ESI): 395.3 ([M+H]⁺).

### b) tert-butyl 8-(3-hydroxyphenyl)-3,8-diazabicyclo[3.2.1]octane-3-carboxylate

To a stirred solution of tert-butyl 8-(3-benzyloxyphenyl)-3,8-diazabicyclo[3.2.1]octane-3-carboxylate (1.94 g, 4.92 mmol, 1.0 eq) in methanol (150 mL) was added ammonium formate (6.2 g, 98.3 mmol, 20 eq). The reaction mixture was degassed with argon for 10 min. 10% Pd on charcoal (523 mg, 492 μmol, 0.1 eq) was added. The reaction mixture was then stirred for 2 h at 70° C. The reaction mixture was filtered and the filtrate was concentrated in vacuo. The residue was taken up in EtOAc/THF (1:1) and washed sequentially with water and brine. The organic layer was dried over Na₂SO₄ and concentrated in vacuo to afford the title compound (1.64 g, 4.9 mmol, 100% yield) as an off-white solid. MS (ESI): 305.1 ([M+H]⁺).

### c) tert-butyl 8-[3-[4-(2-methoxy-2-oxo-ethyl)cyclohexoxy]phenyl]-3,8-diazabicyclo[3.2.1]octane-3-carboxylate

To a stirred solution of tert-butyl 8-(3-hydroxyphenyl)-3,8-diazabicyclo[3.2.1]octane-3-carboxylate (300 mg, 897 μmol, 1.0 eq), methyl 2-((1r,4r)-4-hydroxycyclohexyl)acetate (CAS 1124174-16-8, 309 mg, 1.79 mmol, 2.0 eq) and triphenylphosphine (588 mg, 2.24 mmol, 2.5 eq) in THF (3 mL) at room temperature was added di-tert-butyl azodicarboxylate (454 mg, 1.97 mmol, 2.2 eq). The reaction mixture was stirred at room temperature for 6 h. The reaction mixture was then poured into EtOAc and washed sequentially with water and brine. The organic layer was dried over Na₂SO₄ and concentrated in vacuo. The crude material was purified on silica column (heptane/EtOAc 0-50%) to afford the title compound (637 mg, 1.39 mmol, quantitative yield) as a light yellow waxy solid. MS (ESI): 459.3 ([M+H]⁺).

### d) 2-[4-[3-(3-tert-butoxycarbonyl-3,8-diazabicyclo[3.2.1]octan-8-yl)phenoxy]cyclohexyl]acetic acid

To a stirred solution of tert-butyl 8-[3-[4-(2-methoxy-2-oxo-ethyl)cyclohexoxy]phenyl]-3,8-diazabicyclo[3.2.1]octane-3-carboxylate (400 mg, 872 μmol, 1.0 eq) in THF (4 mL) at room temperature was added 1 M aqueous LiOH solution (2.62 mL, 2.62 mmol, 3.0 eq). The reaction mixture was stirred at room temperature overnight. 5 N aqueous HCl was then added dropwise (to afford pH=1). The reaction mixture was concentrated in vacuo. The product was dissolved in acetonitrile/water (1:3) and lyophilised to afford the title compound (560 mg, 869 μmol, 100% yield). MS (ESI): 445.3 ([M+H]⁺).

### e) tert-butyl 8-[3-[4-[2-(2-benzyloxycarbonyl-2,6-diazaspiro[3.3]heptan-6-yl)-2-oxo-ethyl]cyclohexoxy]phenyl]-3,8-diazabicyclo[3.2.1]octane-3-carboxylate

To a stirred suspension of 2-[4-[3-(3-tert-butoxycarbonyl-3,8-diazabicyclo[3.2.1]octan-8-yl)phenoxy]cyclohexyl]acetic acid (560 mg, 869 μmol, 1.0 eq), benzyl 2,6-diazaspiro[3.3]heptane-2-carboxylate (CAS 1211517-23-5, 222 mg, 956 μmol, 1.1 eq) and DIPEA (225 mg, 304 μL, 1.74 mmol, 2.0 eq) in DMF (2.5 mL) at room temperature was added HATU (496 mg, 1.3 mmol, 1.5 eq). The reaction mixture was stirred at room temperature for 5 h. The product was purified by prep-HPLC to afford the title compound (100 mg, 152 μmol, 18% yield) as a white solid. MS (ESI): 659.7 ([M+H]⁺).

### f) benzyl 6-[2-[4-[3-(3,8-diazabicyclo[3.2.1]octan-8-yl)phenoxy]cyclohexyl]acetyl]-2,6-diazaspiro[3.3]heptane-2-carboxylate

To a stirred solution of tert-butyl 8-[3-[4-[2-(2-benzyloxycarbonyl-2,6-diazaspiro[3.3]heptan-6-yl)-2-oxo-ethyl]cyclohexoxy]phenyl]-3,8-diazabicyclo[3.2.1]octane-3-carboxylate (98 mg, 149 μmol, 1.0 eq) in CH₂Cl₂ (2 mL) at room temperature was added TFA (509 mg, 344 μL, 4.46 mmol, 30.0 eq). The reaction mixture was stirred at room temperature for 2 h. The reaction mixture was concentrated in vacuo, then poured into EtOAc/THF (1:3) and washed with 0.5 N aqueous NaOH/brine (1:1). The organic layer was dried over Na₂SO₄ and concentrated in vacuo to afford the title compound (177 mg, 149 μmol, 100% yield) as a yellow oil. MS (ESI): 559.5 ([M+H]⁺).

### g) benzyl 6-[2-[4-[3-[3-(3-amino-6-chloro-pyridazin-4-yl)-3,8-diazabicyclo[3.2.1]octan-8-yl]phenoxy]cyclohexyl]acetyl]-2,6-diazaspiro[3.3]heptane-2-carboxylate

To a stirred solution of benzyl 6-[2-[4-[3-(3,8-diazabicyclo[3.2.1]octan-8-yl)phenoxy]cyclohexyl]acetyl]-2,6-diazaspiro[3.3]heptane-2-carboxylate (177 mg, 149 μmol, 1.0 eq) and 4-bromo-6-chloropyridazin-3-amine (40.3 mg, 194 μmol, 1.3 eq) in DMSO (0.4 mL) was added K₂CO₃ (123 mg, 893 μmol, 6.0 eq). The reaction mixture was stirred at 110° C. for 15 h. The reaction mixture was poured into EtOAc/THF (1:3) and washed sequentially with water and with brine. The organic layer was dried over Na₂SO₄ and concentrated in vacuo. The crude material was on silica column (DCM/MeOH 0-10%) to afford the title compound (75 mg, 109 μmol, 73% yield) as a brown oil. MS (ESI): 686.3 (³⁵Cl[M+H]⁺).

### h) benzyl 6-[2-[4-[3-[3-[3-amino-6-(2-hydroxyphenyl)pyridazin-4-yl]-3,8-diazabicyclo[3.2.1]octan-8-yl]phenoxy]cyclohexyl]acetyl]-2,6-diazaspiro[3.3]heptane-2-carboxylate

To a stirred solution of benzyl 6-[2-[4-[3-[3-(3-amino-6-chloro-pyridazin-4-yl)-3,8-diazabicyclo[3.2.1]octan-8-yl]phenoxy]cyclohexyl]acetyl]-2,6-diazaspiro[3.3]heptane-2-carboxylate (92 mg, 134 μmol, 1.0 eq) and (2-hydroxyphenyl)boronic acid (46.2 mg, 335 μmol, 2.5 eq) in dioxane (3.5 mL) and water (0.35 mL) was added K₂CO₃ (64.8 mg, 469 μmol, 3.5 eq). The reaction mixture was degassed with argon for 5 min. RuPhos Pd G3 (11.2 mg, 13.4 μmol, 0.1 eq) was added. The reaction mixture was then stirred at 90° C. for 2 h. The reaction mixture was poured into EtOAc/THF (1:1) and washed sequentially with water and brine. The organic layer was dried over Na₂SO₄ and concentrated in vacuo. The crude material was purified on silica column (DCM/MeOH 0-5%) to afford the title compound (38 mg, 51.1 μmol, 38% yield) as a yellow solid. MS (ESI): 744.6 ([M+H]⁺).

### i) 2-[4-[3-[3-[3-amino-6-(2-hydroxyphenyl)pyridazin-4-yl]-3,8-diazabicyclo[3.2.1]octan-8-yl]phenoxy]cyclohexyl]-1-(2,6-diazaspiro[3.3]heptan-2-yl)ethanone

A solution of benzyl 6-[2-[4-[3-[3-[3-amino-6-(2-hydroxyphenyl)pyridazin-4-yl]-3,8-diazabicyclo[3.2.1]octan-8-yl]phenoxy]cyclohexyl]acetyl]-2,6-diazaspiro[3.3]heptane-2-carboxylate (38 mg, 51.1 μmol, 1.0 eq) in methanol (5 mL), THF (2.5 mL) was degassed with argon for 10 min. 10% Pd on charcoal (10.9 mg, 10.2 μmol, 0.2 eq) was added. The reaction mixture was degassed with H₂ for 10 min. The reaction mixture was then stirred under an atmosphere of H₂ at room temperature overnight. The reaction mixture was filtered and the filtrate was concentrated in vacuo to afford the title compound (36 mg, 50.8 μmol, 99% yield) as a brown solid. MS (ESI): 610.5 ([M+H]⁺).

### j) rac-5-[4-[6-[2-[4-[3-[3-[3-amino-6-(2-hydroxyphenyl)pyridazin-4-yl]-3,8-diazabicyclo[3.2.1]octan-8-yl]phenoxy]cyclohexyl]acetyl]-2,6-diazaspiro[3.3]heptane-2-carbonyl]-1-piperidyl]-2-(2,6-dioxo-3-piperidyl)isoindoline-1,3-dione

To a stirred solution of 2-[4-[3-[3-[3-amino-6-(2-hydroxyphenyl)pyridazin-4-yl]-3,8-diazabicyclo[3.2.1]octan-8-yl]phenoxy]cyclohexyl]-1-(2,6-diazaspiro[3.3]heptan-2-yl)ethanone (35 mg, 57.4 μmol, 1.0 eq), Ligase 15 (24.3 mg, 63.1 μmol, 1.1 eq) and DIPEA (18.5 mg, 25.1 μL, 143 μmol, 2.5 eq) in DMF (0.5 mL) at room temperature was added HATU (32.7 mg, 86.1 μmol, 1.5 eq). The reaction mixture was stirred at room temperature for 3 h. The crude material was purified by prep-HPLC to afford the title compound (8 mg, 8.2 μmol, 14% yield) as a yellow solid. MS (ESI): 977.5 ([M+H]⁺).

### Example 32

### 4-[[10-[4-[[3-[1-[3-amino-6-(2-hydroxyphenyl)pyridazin-4-yl]azetidin-3-yl]oxyphenyl]methyl]piperazin-1-yl]-10-oxo-decyl]amino]-2-(2,6-dioxo-3-piperidyl)isoindoline-1,3-dione

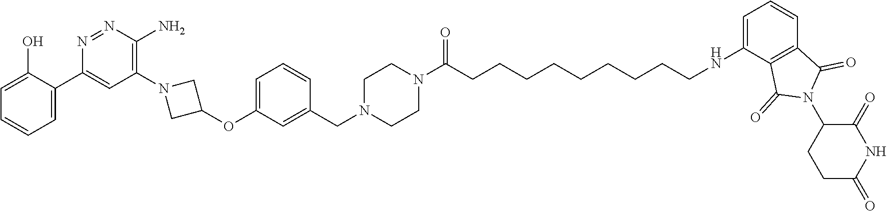 [SMILES: Nc1nnc(-c2ccccc2O)cc1N1CC(Oc2cccc(CN3CCN(C(=O)CCCCCCCCCNc4cccc5c4C(=O)N(C4CCC(=O)NC4=O)C5=O)CC3)c2)C1]

A mixture of 2-[6-amino-5-[3-[3-(piperazin-1-ylmethyl)phenoxy]azetidin-1-yl]pyridazin-3-yl]phenol (30 mg, 0.05 mmol, 1.0 eq), Ligase 23 (27 mg, 0.06 mmol, 1.2 eq), Et₃N (0.5 mL, 0.15 mmol, 3.0 eq) and T₃P (25 mg, 0.06 mmol, 1.2 eq) in DMF (3 mL) was stirred at 25° C. for 1 h. The reaction mixture was purified by prep-HPLC to afford the title compound (14.5 mg, 16.9 mmol, 32% yield) as a yellow solid. MS (ESI): 858.4 ([M+H]⁺).

### Example 33

### rac-5-[4-[2-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-2-oxo-ethyl]-1-piperidyl]-2-(2,6-dioxo-3-piperidyl)isoindoline-1,3-dione

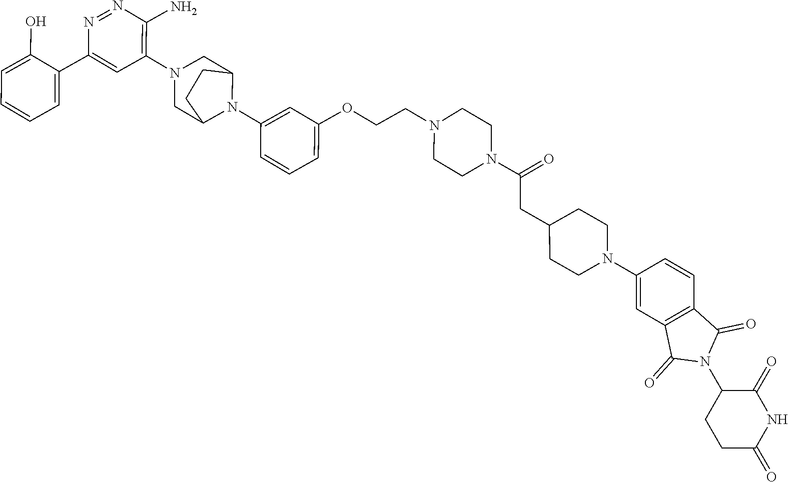 [SMILES: Nc1nnc(-c2ccccc2O)cc1N1CC2CCC(C1)N2c1cccc(OCCN2CCN(C(=O)CC3CCN(c4ccc5c(c4)C(=O)N(C4CCC(=O)NC4=O)C5=O)CC3)CC2)c1]

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (25 mg, 49.8 μmol, 1.0 eq), Ligase 24 (19.9 mg, 49.8 μmol, 1.0 eq) and HATU (41.7 mg, 110 μmol, 2.2 eq) in DMF (0.5 mL) at room temperature was added DIPEA (19.3 mg, 26.1 μL, 150 μmol, 3.0 eq). The reaction mixture was stirred for 2 h. The crude material was purified by prep-HPLC to afford the title compound (15.9 mg, 14.3 mmol, 29% yield) as a yellow solid, bis-(2,2,2-trifluoroacetate) salt. MS (ESI): 883.4 ([M+H]⁺).

### Example 34

### rac-5-[4-[2-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]ethyl]-1-piperidyl]-2-(2,6-dioxo-3-piperidyl)isoindoline-1,3-dione

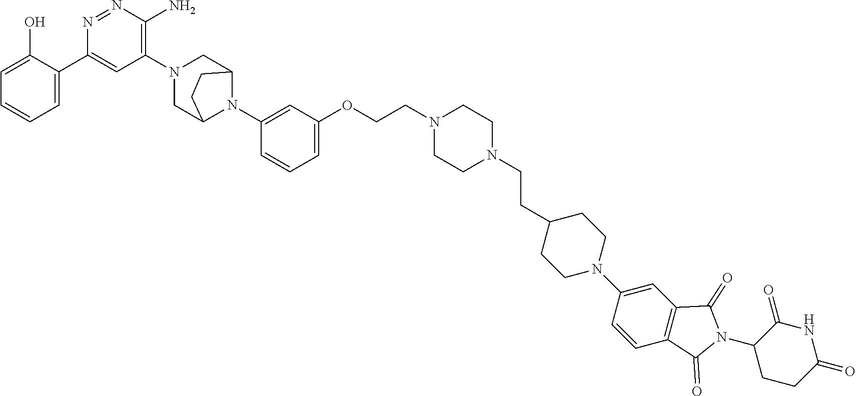 [SMILES: Nc1nnc(-c2ccccc2O)cc1N1CC2CCC(C1)N2c1cccc(OCCN2CCN(CCC3CCN(c4ccc5c(c4)C(=O)N(C4CCC(=O)NC4=O)C5=O)CC3)CC2)c1]

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (30 mg, 59.8 μmol, 1.0 eq) and Ligase 25 (26.8 mg, 59.8 μmol, 1.0 eq) in DMF (0.5 mL) at room temperature was added sodium iodide (896 μg, 5.98 μmol, 0.1 eq). The reaction mixture was heated at 60° C. for 20 h. The crude material was purified by prep-HPLC to afford the title compound (28.2 mg, 25.7 μmol, 43% yield) as a yellow soliud, bis-(2,2,2-trifluoroacetic acid)salt. MS (ESI): 869.4 ([M+H]⁺).

### Example 35

### rac-5-[4-[2-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]ethoxy]-1-piperidyl]-2-(2,6-dioxo-3-piperidyl)isoindoline-1,3-dione

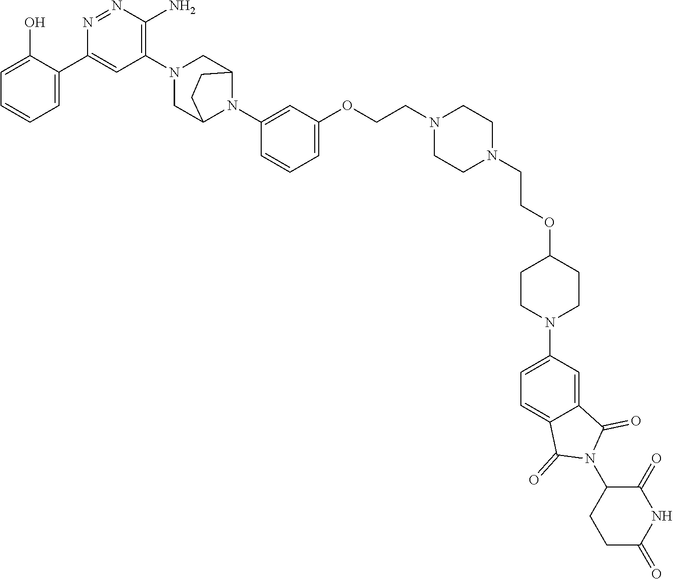 [SMILES: Nc1nnc(-c2ccccc2O)cc1N1CC2CCC(C1)N2c1cccc(OCCN2CCN(CCOC3CCN(c4ccc5c(c4)C(=O)N(C4CCC(=O)NC4=O)C5=O)CC3)CC2)c1]

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (30 mg, 59.8 μmol, 1.0 eq) and Ligase 26 (27.8 mg, 59.8 μmol, 1.0 eq) in DMF (0.5 mL) at room temperature was added sodium iodide (896 μg, 5.98 μmol, 0.1 eq). The reaction mixture was heated at 60° C. for 20 h. The crude material was purified by prep-HPLC to afford the title compound (24.9 mg, 22.4 μmol, 37% yield) as a yellow solid, bis-(2,2,2-trifluoroacetic acid)salt. MS (ESI): 885.4 ([M+H]⁺).

### Example 36

### rac-5-[4-[3-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]propoxy]-1-piperidyl]-2-(2,6-dioxo-3-piperidyl)isoindoline-1,3-dione

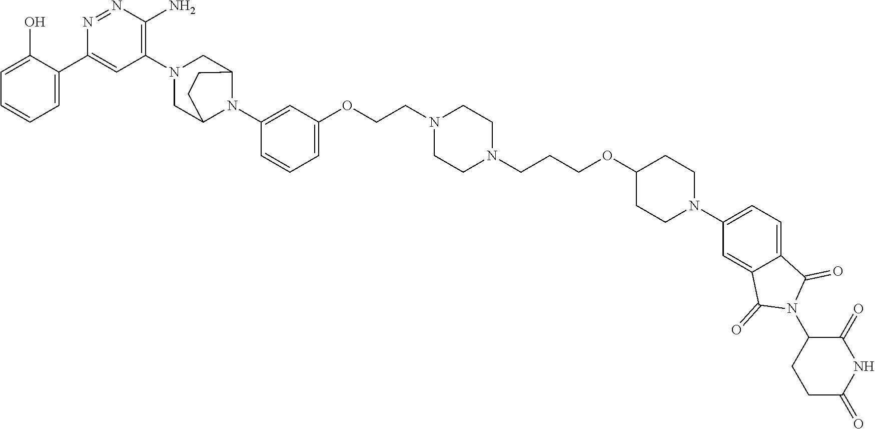 [SMILES: Nc1nnc(-c2ccccc2O)cc1N1CC2CCC(C1)N2c1cccc(OCCN2CCN(CCCOC3CCN(c4ccc5c(c4)C(=O)N(C4CCC(=O)NC4=O)C5=O)CC3)CC2)c1]

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (30 mg, 59.8 μmol, 1.0 eq) and Ligase 27 (28.6 mg, 59.8 μmol, 1.0 eq) in DMF (0.5 mL) at room temperature was added sodium iodide (896 μg, 5.98 μmol, 0.1 eq). The reaction mixture was heated at 60° C. for 20 h. The crude material was purified by prep-HPLC to afford the title compound (24.7 mg, 21.9 μmol, 37% yield) as a yellow solid, bis-(2,2,2-trifluoroacetic acid)salt. MS (ESI): 899.5 ([M+H]⁺).

### Example 37

### 4-[[1-[9-[4-[4-[3-[3-amino-6-(2-hydroxyphenyl)pyridazin-4-yl]oxy-1-piperidyl]phenyl]piperazin-1-yl]nonyl]triazol-4-yl]methylamino]-2-(2,6-dioxo-3-piperidyl)isoindoline-1,3-dione

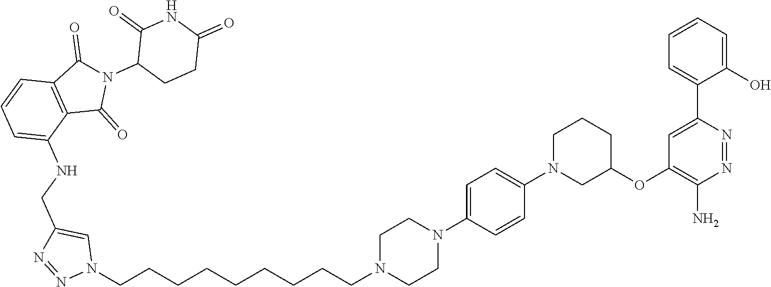 [SMILES: Nc1nnc(-c2ccccc2O)cc1OC1CCCN(c2ccc(N3CCN(CCCCCCCCCn4cc(CNc5cccc6c5C(=O)N(C5CCC(=O)NC5=O)C6=O)nn4)CC3)cc2)C1]

To a solution of Ligase 28 (20 mg, 40.44 μmol) and 2-[6-amino-5-[[1-(4-piperazin-1-ylphenyl)-3-piperidyl]oxy]pyridazin-3-yl]phenol (18 mg, 40.44 μmol) in methanol (5 mL) was added acetic acid (243 ug, 4.04 umol, 2.31 e-1 uL) and Biotage® MP-cyanoborohydride (50 mg, 40.44 μmol). It was stirred for 16 h at 80° C. The reaction mixture was cooled to room temperature, filtered and washed with methanol. The organic layer was concentrated under reduced pressure. The crude residue was purified by prep-HPLC to afford the title compound (10 mg, 10.6 μmol, 26% yield) as a pale yellow solid. MS (ESI): 925.4 ([M+H]⁺).

### Example 38

### rac-5-[4-[[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]methyl]-1-piperidyl]-2-(2,6-dioxo-3-piperidyl)isoindoline-1,3-dione

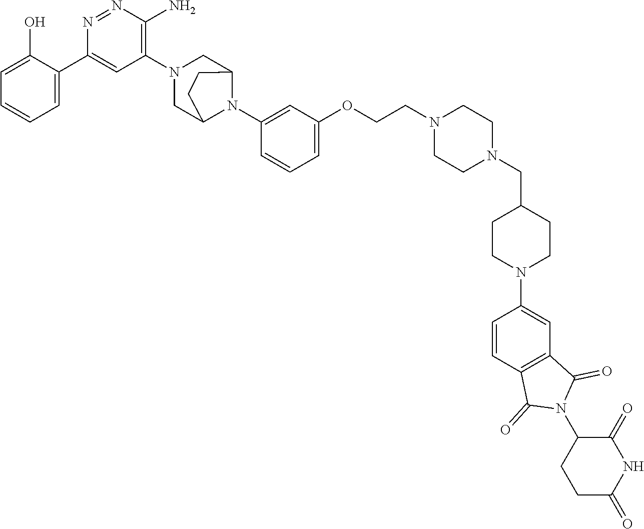 [SMILES: Nc1nnc(-c2ccccc2O)cc1N1CC2CCC(C1)N2c1cccc(OCCN2CCN(CC3CCN(c4ccc5c(c4)C(=O)N(C4CCC(=O)NC4=O)C5=O)CC3)CC2)c1]

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (30 mg, 59.8 μmol, 1.0 eq) and Ligase 33 (26 mg, 59.8 μmol, 1.0 eq) in DMF (0.5 mL) at room temperature was added sodium iodide (896 μg, 5.98 μmol, 0.1 eq). The reaction mixture was heated at 60° C. for 20 h. The crude material was purified by prep-HPLC to afford the title compound (26.2 mg, 24.2 μmol, 40% yield) as a yellow solid, bis-(2,2,2-trifluoroacetic acid)salt. MS (ESI): 855.4 ([M+H]⁺).

### Example 39

### 4-[[10-[4-[3-[1-[3-amino-6-(2-hydroxyphenyl)pyridazin-4-yl]azetidin-3-yl]oxyphenyl]piperazin-1-yl]-10-oxo-decyl]amino]-2-(2,6-dioxo-3-piperidyl)isoindoline-1,3-dione

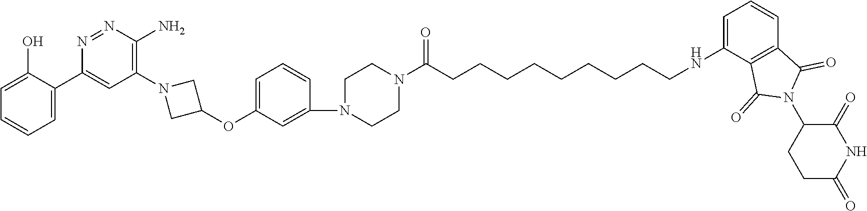 [SMILES: Nc1nnc(-c2ccccc2O)cc1N1CC(Oc2cccc(N3CCN(C(=O)CCCCCCCCCNc4cccc5c4C(=O)N(C4CCC(=O)NC4=O)C5=O)CC3)c2)C1]

### a) benzyl 4-(3-hydroxyphenyl)piperazine-1-carboxylate

To a solutions of 1-(3-hydroxyphenyl)piperazine (5 g, 28.05 mmol, 1.0 eq), potassium bicarbonate (4.5 g, 44.89 mmol, 1.6 eq) in acetone (50 mL) and water (50 mL) was added benzyl chloroformate (7.18 g, 42.08 mmol, 1.5 eq) at 0° C., the mixture was stirred at 0° C. for 2 h. The reaction mixture was poured into water, extracted with EtOAc, dried over sodium sulfate and concentrated in vacuum. The crude product was purified by prep-HPLC to afford the title compound (4 g, 12.8 mmol, 45% yield) as a yellow oil. MS (ESI): 313.2 ([M+H]⁺).

### b) benzyl 4-(3-((1-(tert-butoxycarbonyl)azetidin-3-yl)oxy)phenyl)piperazine-1-carboxylate

A mixture of 1-boc-3-iodoazetidine (4.7 g, 16.65 mmol, 1.3 eq), benzyl 4-(3-hydroxyphenyl)piperazine-1-carboxylate (4 g, 12.81 mmol, 1.0 eq), cesium carbonate (7.5 g, 23.05 mmol, 2.0 eq) in DMF (80 mL) was stirred at 80° C. for 12 h. The reaction mixture was poured into water, extracted with EtOAc, dried over Na₂SO₄ and concentrated in vacuum to afford the title compound (4 g, 8.56 mmol, 66% yield) as a yellow oil. MS (ESI): 468.2 ([M+H]⁺)

### c) benzyl 4-(3-(azetidin-3-yloxy)phenyl)piperazine-1-carboxylate

A mixture of benzyl 4-[3-(1-tert-butoxycarbonylazetidin-3-yl)oxyphenyl]piperazine-1-carboxylate (4 g, 8.56 mmol, 1.0 eq) in TFA (10 mL, 89.7 mmol, 10 eq) and DCM (50 mL) was stirred at 25° C. for 2 h. The reaction mixture was concentrated to afford the title compound (3 g, 8.17 mmol, 95% yield) as a yellow oil. MS (ESI): 368.2 ([M+H]⁺)

### d) benzyl 4-(3-((1-(3-amino-6-chloropyridazin-4-yl)azetidin-3-yl)oxy)phenyl)piperazine-1-carboxylate

A mixture of benzyl 4-[3-(azetidin-3-yloxy)phenyl]piperazine-1-carboxylate (3 g, 8.16 mmol, 1.0 eq), 4-bromo-6-chloro-pyridazin-3-amine (2.04 g, 9.8 mmol, 1.2 eq), Et₃N (3.3 g, 32.66 mmol, 4.0 eq) in DMF (50 mL) was stirred at 100° C. for 12 h. The reaction mixture was purified by prep-HPLC to afford the title compound (2 g, 4.05 mmol, 49% yield) as a yellow oil. MS (ESI): 495.2 ([M+H]⁺).

### e) benzyl 4-(3-((1-(3-amino-6-(2-hydroxyphenyl)pyridazin-4-yl)azetidin-3-yl)oxy)phenyl) piperazine-1-carboxylate

A mixture of Ruphos-Pd-G3 (50 mg, 0.030 mmol, 0.03 eq), K₂CO₃ (489 mg, 3.54 mmol, 3.0 eq), benzyl 4-[3-[1-(3-amino-6-chloro-pyridazin-4-yl)azetidin-3-yl]oxyphenyl]piperazine-1-carboxylate (500 mg, 1.01 mmol, 1.0 eq), 2-hydroxyphenylboronic acid (348 mg, 2.53 mmol, 2.5 eq) in 1,4-dioxane (10 mL) and water (1 mL) was stirred at 90° C. for 2 h under N₂ atmosphere. The reaction mixture was purified by prep-HPLC to afford the title compound (300 mg, 0.54 mmol, 52% yield) as a white solid. MS (ESI): 553.3 ([M+H]⁺).

### f) 2-(6-amino-5-(3-(3-(piperazin-1-yl)phenoxy)azetidin-1-yl)pyridazin-3-yl)phenol

A mixture of benzyl 4-[3-[1-[3-amino-6-(2-hydroxyphenyl)pyridazin-4-yl]azetidin-3-yl]oxyphenyl]piperazine-1-carboxylate (250 mg, 0.45 mmol, 1.0 eq) and 10% palladium on active carbonate (100 mg) was stirred in methanol (10 mL) under H₂ atmosphere (15 psi) over night. The reaction mixture was filtered and the filtrate was purified by prep-HPLC to afford the title compound (120 mg, 0.28 mmol, 62% yield) as a white solid. MS (ESI): 419.3 ([M+H]⁺).

### g) 4-((10-(4-(3-((1-(3-amino-6-(2-hydroxyphenyl)pyridazin-4-yl)azetidin-3-yl)oxy)phenyl)piperazin-1-yl)-10-oxodecyl)amino)-2-(2,6-dioxopiperidin-3-yl)isoindoline-1,3-dione

A mixture of Ligase 23 (25 mg, 0.06 mmol, 1.2 eq), 2-[6-amino-5-[3-(3-piperazin-1-ylphenoxy)azetidin-1-yl]pyridazin-3-yl]phenol (20 mg, 0.05 mmol, 1.0 eq), Et₃N (0.5 mL, 0.14 mmol, 3.0 eq) and T₃P (27 mg, 0.06 mmol, 1.2 eq) in DMF (2 mL) was stirred at 25° C. for 1 h. The reaction mixture was purified by prep-HPLC to afford the title compound (3.2 mg, 3.79 μmol, 7.8% yield) as a yellow solid. MS (ESI): 844.5 ([M+H]⁺).

### Example 40

### rac-5-[[1-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]-4-piperidyl]oxy]-2-(2,6-dioxo-3-piperidyl)isoindoline-1,3-dione

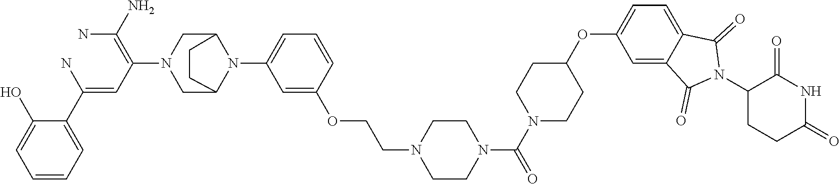 [SMILES: NC(N)=C(/C=C(\N)c1ccccc1O)N1CC2CCC(C1)N2c1cccc(OCCN2CCN(C(=O)N3CCC(Oc4ccc5c(c4)C(=O)N(C4CCC(=O)NC4=O)C5=O)CC3)CC2)c1]

### a) (4-nitrophenyl) 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate

A stirred solution of 2-[6-amino-5-[8-[3-(2-piperazin-1-ylethoxy)phenyl]-3,8-diazabicyclo[3.2.1]octan-3-yl]pyridazin-3-yl]phenol (100 mg, 199 μmol, 1.0 eq) in DCM (8 mL) was cooled to 0° C. 4-Nitrophenyl carbonochloridate (40.2 mg, 199 μmol, 1.0 eq) was added. The reaction mixture was stirred at room temperature for 30 min. The reaction mixture was then poured into EtOAc/THF (2:1) and washed sequentially with water and with brine. The organic layer was dried over Na₂SO₄ and concentrated in vacuo. The crude material was purified on silica column (DCM/MeOH 0-5%) to afford the title compound (64 mg, 96 μmol, 48% yield) as a yellow solid. MS (ESI): 667.3 ([M+H]⁺).

### b) rac-5-[[1-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]-4-piperidyl]oxy]-2-(2,6-dioxo-3-piperidyl)isoindoline-1,3-dione

To a stirred suspension of (4-nitrophenyl) 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate (30 mg, 45 μmol, 1.0 eq) and Ligase 8 (19.5 mg, 49.5 μmol, 1.1 eq, HCl salt) in acetonitrile (0.5 mL) at room temperature was added DIPEA (14.5 mg, 19.6 μL, 112 μmol, 2.5 eq) and DMSO (0.5 mL) to afford a yellow solution. The reaction mixture was stirred at 100° C. overnight then at 110° C. for 24 h. The crude material was purified by prep-HPLC followed on silica column (DCM/MeOH 0-5%) to afford the title compound (7 mg, 7.9 μmol, 18% yield) as a white solid. MS (ESI): 885.4 ([M+H]⁺).

### Example 41

### rac-4-[[1-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]-4-piperidyl]oxy]-2-(2,6-dioxo-3-piperidyl)isoindoline-1,3-dione

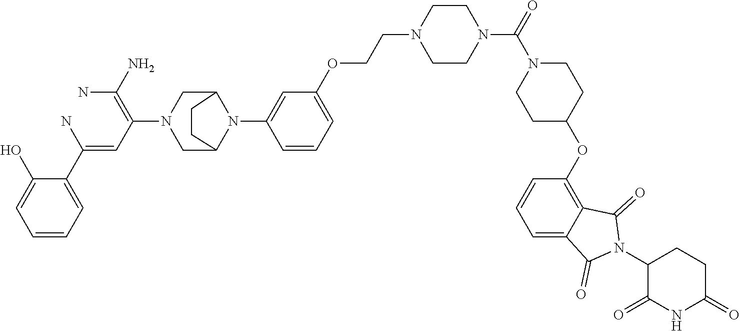 [SMILES: NC(N)=C(/C=C(\N)c1ccccc1O)N1CC2CCC(C1)N2c1cccc(OCCN2CCN(C(=O)N3CCC(Oc4cccc5c4C(=O)N(C4CCC(=O)NC4=O)C5=O)CC3)CC2)c1]

To a stirred solution of (4-nitrophenyl) 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate (29 mg, 43.5 μmol, 1.0 eq) and Ligase 7 (18.8 mg, 47.8 μmol, 1.1 eq, HCl salt) in DMSO (0.5 mL) at room temperature was added DIPEA (14.1 mg, 19 μL, 109 μmol, 2.5 eq). The reaction mixture was stirred at 110° C. for 24 h. The crude material was purified by prep-HPLC to afford the title compound (13 mg, 11.7 μmol, 27% yield) as a light yellow solid, bis-(2,2,2-trifluoroacetate) salt. MS (ESI): 885.4 ([M+H]⁺).

### Example 42

### rac-N-[3-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-3-oxo-propyl]-1-[2-(2,6-dioxo-3-piperidyl)-1,3-dioxo-isoindolin-5-yl]piperidine-4-carboxamide

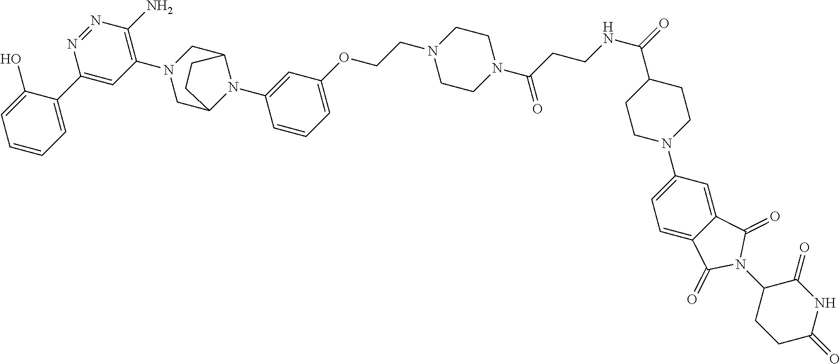 [SMILES: Nc1nnc(-c2ccccc2O)cc1N1CC2CCC(C1)N2c1cccc(OCCN2CCN(C(=O)CCNC(=O)C3CCN(c4ccc5c(c4)C(=O)N(C4CCC(=O)NC4=O)C5=O)CC3)CC2)c1]

### a) tert-butyl N-[3-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-3-oxo-propyl]carbamate

To a stirred suspension of 2-[6-amino-5-[8-[3-(2-piperazin-1-ylethoxy)phenyl]-3,8-diazabicyclo[3.2.1]octan-3-yl]pyridazin-3-yl]phenol (50 mg, 99.7 μmol, 1.0 eq), 3-((tert-butoxycarbonyl)amino)propanoic acid (20.7 mg, 110 μmol, 1.1 eq) and DIPEA (38.6 mg, 52.2 μL, 299 μmol, 3.0 eq) in DMF (0.6 mL) at room temperature was added HATU (75.8 mg, 199 μmol, 2.0 eq). The reaction mixture was stirred at room temperature for 3 h. The reaction mixture was poured into EtOAc/THF (1:2) and washed sequentially with water and with brine. The organic layer was dried over Na₂SO₄ and concentrated in vacuo. The material was purified on silica column (DCM/MeOH 0-10%) to afford the title compound (9 mg, 13.4 μmol, 13% yield) as a yellow solid. MS (ESI): 671.7 ([M−H]⁻).

### b) 3-amino-1-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]propan-1-one

To a stirred mixture of tert-butyl N-[3-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-3-oxo-propyl]carbamate (44 mg, 65.4 μmol, Eq: 1) in dioxane (2 mL) at room temperature was added 4 M HCl in dioxane (490 μL, 1.96 mmol, 30.0 eq). The reaction mixture was stirred at room temperature for 3 h. The reaction mixture was filtered through sintered glass and the filter cake was washed twice with Et₂O, then dried in vacuo to afford the title compound (36 mg, 52.8 μmol, 81% yield) as a yellow solid, trihydrochloride salt. MS (ESI): 571.6 ([M−H]⁻).

### c) rac-N-[3-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-3-oxo-propyl]-1-[2-(2,6-dioxo-3-piperidyl)-1,3-dioxo-isoindolin-5-yl]piperidine-4-carboxamide

To a stirred solution of 3-amino-1-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]propan-1-one trihydrochloride (36 mg, 52.8 μmol, 1.0 eq), Ligase 15 (22.4 mg, 58.1 μmol, 1.1 eq) and DIPEA (40.9 mg, 55.3 μL, 317 μmol, 6.0 eq) in DMF (0.6 mL) at room temperature was added HATU (40.1 mg, 106 μmol, 2.0 eq). The reaction mixture was stirred at room temperature for 1 h. The crude material was purified by prep-HPLC to afford the title compound (20 mg, 17.1 μmol, 32% yield) as a yellow solid, bis-(2,2,2-trifluoroacetic acid)salt. MS (ESI): 940.4 ([M+H]⁺).

### Example 43

### rac-3-[4-[1-[2-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-2-oxo-ethyl]-4-piperidyl]phenoxy]piperidine-2,6-dione

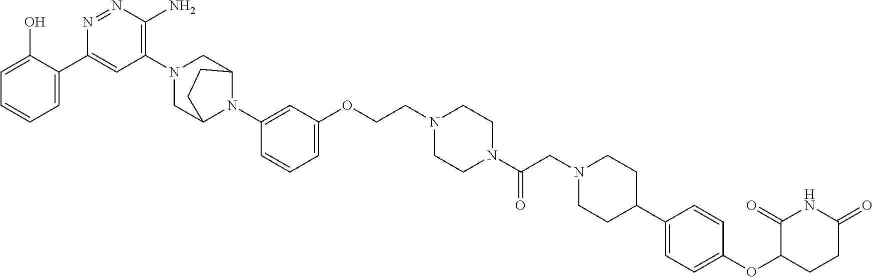 [SMILES: Nc1nnc(-c2ccccc2O)cc1N1CC2CCC(C1)N2c1cccc(OCCN2CCN(C(=O)CN3CCC(c4ccc(OC5CCC(=O)NC5=O)cc4)CC3)CC2)c1]

To a stirred suspension of 2-[6-amino-5-[8-[3-(2-piperazin-1-ylethoxy)phenyl]-3,8-diazabicyclo[3.2.1]octan-3-yl]pyridazin-3-yl]phenol (30 mg, 59.8 μmol, 1.0 eq), Ligase 35 (25.2 mg, 65.8 μmol, 1.1 eq) and DIPEA (38.6 mg, 52.2 μL, 299 μmol, 5.0 eq) in DMF (0.5 mL) at room temperature was added HATU (45.5 mg, 120 μmol, 2.0 eq). The reaction mixture was stirred at room temperature for 1 h. The crude material was purified by prep-HPLC. The product was lyophilised to afford the title compound (30 mg, 28.4 μmol, 47% yield) as a light yellow solid, bis-(2,2,2-trifluoroacetate) salt. MS (ESI): 830.4 ([M+H]⁺).

### Example 44

### 5-[[1-[9-[4-[4-[3-[3-amino-6-(2-hydroxyphenyl)pyridazin-4-yl]oxy-1-piperidyl]phenyl]piperazin-1-yl]-9-oxo-nonyl]-4-piperidyl]oxy]-2-(2,6-dioxo-3-piperidyl)isoindoline-1,3-dione

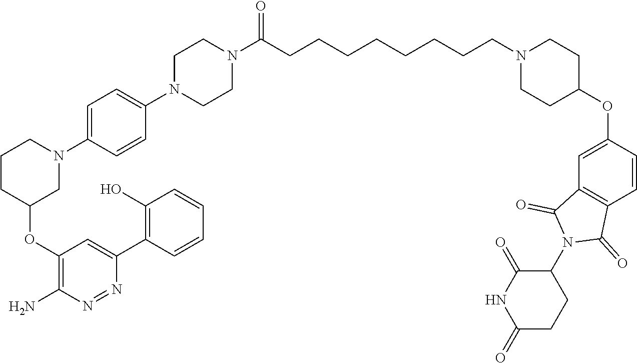 [SMILES: Nc1nnc(-c2ccccc2O)cc1OC1CCCN(c2ccc(N3CCN(C(=O)CCCCCCCCN4CCC(Oc5ccc6c(c5)C(=O)N(C5CCC(=O)NC5=O)C6=O)CC4)CC3)cc2)C1]

To a stirred solution of Ligase 34 (19.0 mg, 37.0 μmol) and 2-[6-amino-5-[[1-(4-piperazin-1-ylphenyl)-3-piperidyl]oxy]pyridazin-3-yl]phenol (20 mg, 37.0 μmol, TFA salt) in DMF (0.5 mL) was added HATU (21 mg, 55.5 μmol) followed by DIPEA (24 mg, 185 μmol, 32.22 μL) and the reaction mixture was stirred at room temperature for 16 h. The reaction mixture was diluted with water and extracted with EtOAc. The volatiles were removed. The crude residue was purified by prep-HPLC to afford the title compound (7.7 mg, 7.3 μmol, 19% yield) as an off-white solid. MS (ESI): 942.3 ([M+H]⁺).

### Example 45

### 2-(2,6-dioxo-3-piperidyl)-4-[[rac-(3S)-1-[9-[4-[4-[3-[3-amino-6-(2-hydroxyphenyl)pyridazin-4-yl]oxy-1-piperidyl]phenyl]piperazin-1-yl]-9-oxo-nonanoyl]pyrrolidin-3-yl]amino]isoindoline-1,3-dione

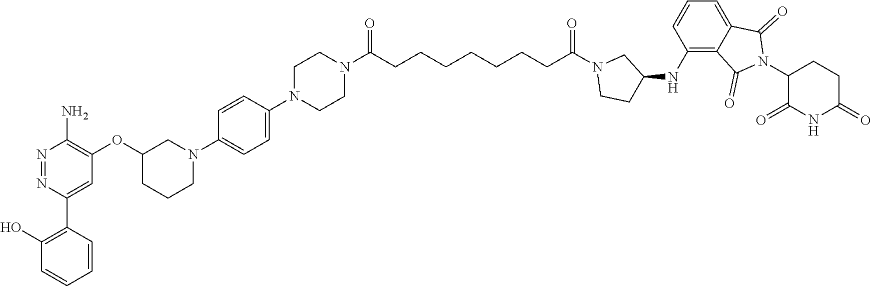 [SMILES: Nc1nnc(-c2ccccc2O)cc1OC1CCCN(c2ccc(N3CCN(C(=O)CCCCCCCC(=O)N4CC[C@H](Nc5cccc6c5C(=O)N(C5CCC(=O)NC5=O)C6=O)C4)CC3)cc2)C1]

The title compound was prepared in analogy to example 3 step e using Ligase 36 (7.4 mg, 7.3 μmol, 19% yield) as a light yellow solid, trifluoroacetic acid salt. MS (ESI): 941.3 ([M+H]⁺).

### Example 46

### rac-5-[4-[4-[[3-[3-[3-Amino-6-(2-hydroxyphenyl)pyridazin-4-yl]-3,8-diazabicyclo[3.2.1]octan-8-yl]phenyl]methyl]piperazine-1-carbonyl]-1-piperidyl]-2-(2,6-dioxo-3-piperidyl)isoindoline-1,3-dione

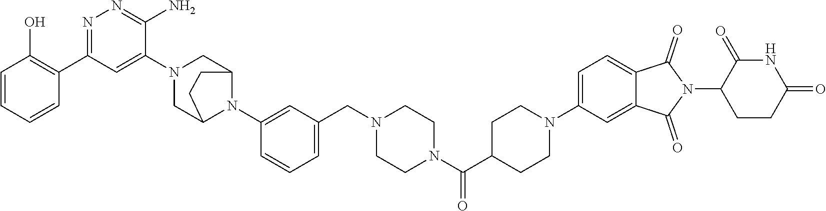 [SMILES: Nc1nnc(-c2ccccc2O)cc1N1CC2CCC(C1)N2c1cccc(CN2CCN(C(=O)C3CCN(c4ccc5c(c4)C(=O)N(C4CCC(=O)NC4=O)C5=O)CC3)CC2)c1]

### a) Benzyl 4-[(3-bromophenyl)methyl]piperazine-1-carboxylate

To a solution of 1-bromo-3-(bromomethyl)benzene (2 g, 8 mmol, 1.0 eq) and triethylamine (1.21 g, 1.67 mL, 12 mmol, 1.5 eq) in THF (40 mL) was added benzyl piperazine-1-carboxylate (2.12 g, 1.85 mL, 9.6 mmol, 1.2 eq) and the reaction mixture was stirred at ambient temperature for 16 h. The crude reaction mixture was concentrated in vacuo and the crude product purified on silica column (heptane/EtOAc 0-100%) to afford the title compound (3.1 g, 7.9 mmol, 99% yield) as a colourless gum. MS (ESI): 391.1 ([M+H]⁺).

### b) tert-Butyl 8-[3-[(4-benzyloxycarbonylpiperazin-1-yl)methyl]phenyl]-3,8-diazabicyclo[3.2.1]octane-3-carboxylate

A solution of Pd(OAc)₂ (57.7 mg, 257 μmol, 0.1 eq) and 2-dicyclohexylphosphino-2′,6′-diisopropoxybiphenyl (Ruphos) (240 mg, 514 μmol, 0.2 eq) in toluene (5 mL) was degassed by purging with argon, followed by heating of the solution to 50° C. for 20 min. In a separate vessel, tert-butyl 3,8-diazabicyclo[3.2.1]octane-3-carboxylate (654 mg, 3.08 mmol, 1.2 eq), benzyl 4-[(3-bromophenyl)methyl]piperazine-1-carboxylate (1 g, 2.57 mmol, 1.0 eq) and sodium tert-butoxide (370 mg, 3.85 mmol, 1.5 eq) were dissolved in toluene (5 mL) and degassed by purging with argon. The catalyst solution was added to the reaction vessel and the resulting mixture heated to 110° C. for 16 h. The reaction mixture was filtered through celite, concentrated in vacuo and the crude material was purified by silica gel flash chromatography using DCM (+0.5% TEA)/MeOH (0-10%) as eluent to afford the title compound (936 mg, 1.79 mmol, 70% yield) as a brown oil. MS (ESI): 521.3143 ([M+H]⁺).

### c) Benzyl 4-[[3-(3,8-diazabicyclo[3.2.1]octan-8-yl)phenyl]methyl]piperazine-1-carboxylate

Tert-butyl 8-[3-[(4-benzyloxycarbonylpiperazin-1-yl)methyl]phenyl]-3,8-diazabicyclo[3.2.1]octane-3-carboxylate (860 mg, 1.65 mmol, 1.0 eq) was dissolved in DCM (10 mL) and TFA (7.4 g, 5 mL, 64.9 mmol, 39.3 eq) was slowly added. The reaction mixture was stirred at ambient temperature for 16 h. The reaction mixture was concentrated in vacuo to afford the title compound (1.4 g, 3.33 mmol, 131% yield) as a brown solid, bis-(2,2,2-trifluoroacetic acid) salt. MS (ESI): 421.3 ([M+H]⁺).

### d) Benzyl 4-[[3-[3-(3-amino-6-chloro-pyridazin-4-yl)-3,8-diazabicyclo[3.2.1]octan-8-yl]phenyl]methyl]piperazine-1-carboxylate

To a solution of benzyl 4-[[3-(3,8-diazabicyclo[3.2.1]octan-8-yl)phenyl]methyl]piperazine-1-carboxylate; bis-(2,2,2-trifluoroacetic acid) (1.4 g, 2.7 mmol, 1.0 eq) in DMSO (6 mL) was added 4-bromo-6-chloropyridazin-3-amine (619 mg, 2.97 mmol, 1.1 eq) and potassium carbonate (1.87 g, 13.5 mmol, 5.0 eq). The reaction mixture was heated to 100° C. for 48 h. The reaction mixture was poured into water, extracted with EtOAc, washed with brine and the organic layer was dried over Na₂SO₄ and concentrated in vacuo. The crude material was purified on silica column (DCM+0.5% TEA/MeOH 0-10%) to afford the title compound (550 mg, 1.0 mmol, 37% yield) as a brown oil. MS (ESI): 548.3 ([M+H]⁺).

### e) Benzyl 4-[[3-[3-[3-amino-6-(2-hydroxyphenyl)pyridazin-4-yl]-3,8-diazabicyclo[3.2.1]octan-8-yl]phenyl]methyl]piperazine-1-carboxylate

To a solution of benzyl 4-[[3-[3-(3-amino-6-chloro-pyridazin-4-yl)-3,8-diazabicyclo[3.2.1]octan-8-yl]phenyl]methyl]piperazine-1-carboxylate (388 mg, 708 μmol, 1.0 eq) in a mixture of dioxane (3 mL), DMA (0.15 mL) and water (0.3 mL) was added (2-hydroxyphenyl)boronic acid (244 mg, 1.77 mmol, 2.5 eq) and potassium carbonate (294 mg, 2.12 mmol, 3.0 eq). The solution was degassed by purging with argon and after addition of RuPhos Pd G3 (29.6 mg, 35.4 μmol, 0.05 eq) the reaction mixture was stirred at 90° C. for 5 h. The reaction mixture was poured into saturated NH₄Cl solution, extracted with EtOAc, washed with brine and the organic layer was dried over Na₂SO₄ and concentrated in vacuo. The crude material was purified on silica column (DCM+0.5% TEA/MeOH 0-10%) to afford the title compound (420 mg, 693 μmol, 76% yield) as a brown gum. MS (ESI): 606.5 ([M+H]⁺).

### f) 2-[6-Amino-5-[8-[3-(piperazin-1-ylmethyl)phenyl]-3,8-diazabicyclo[3.2.1]octan-3-yl]pyridazin-3-yl]phenol

To a solution of benzyl 4-[[3-[3-[3-amino-6-(2-hydroxyphenyl)pyridazin-4-yl]-3,8-diazabicyclo[3.2.1]octan-8-yl]phenyl]methyl]piperazine-1-carboxylate (16 mg, 26.4 μmol, 1.0 eq) in DCM (1 mL) was added carefully HBr (64.8 mg, 43.5 μL, 264 μmol, 10 eq; 33% in acetic acid) and the reaction mixture was stirred at ambient temperature for 2 h. The reaction mixture was concentrated in vacuo to afford the title compound (14 mg, 29.8 μmol, 96% yield) as a light brown solid, hydrobromide salt. MS (ESI): 470.4 ([M+H]⁺).

### g) rac-5-[4-[4-[[3-[3-[3-Amino-6-(2-hydroxyphenyl)pyridazin-4-yl]-3,8-diazabicyclo[3.2.1]octan-8-yl]phenyl]methyl]piperazine-1-carbonyl]-1-piperidyl]-2-(2,6-dioxo-3-piperidyl)isoindoline-1,3-dione

2-[6-Amino-5-[8-[3-(piperazin-1-ylmethyl)phenyl]-3,8-diazabicyclo[3.2.1]octan-3-yl]pyridazin-3-yl]phenol; hydrobromide (35 mg, 63.3 μmol, 1.0 eq) was combined with Ligase 15 (26.9 mg, 69.7 μmol, 1.1 eq), HATU (36.1 mg, 95 μmol, 1.5 eq) and DIPEA (40.9 mg, 55 μL, 317 μmol, 5.0 eq) in DMF (350 μL). The dark orange solution was stirred at ambient temperature for 8 h and was then submitted to prep-HPLC for purification to afford the title compound (14 mg, 16.7 μmol, 23% yield) as a light yellow solid, 2,2,2-trifluoroacetate salt. MS (ESI): 839.4 ([M+H]⁺).

### Example 47

### rac-5-[4-[3-[4-[[3-[3-[3-Amino-6-(2-hydroxyphenyl)pyridazin-4-yl]-3,8-diazabicyclo[3.2.1]octan-8-yl]phenyl]methyl]piperazin-1-yl]-3-oxo-propyl]-1-piperidyl]-2-(2,6-dioxo-3-piperidyl)isoindoline-1,3-dione

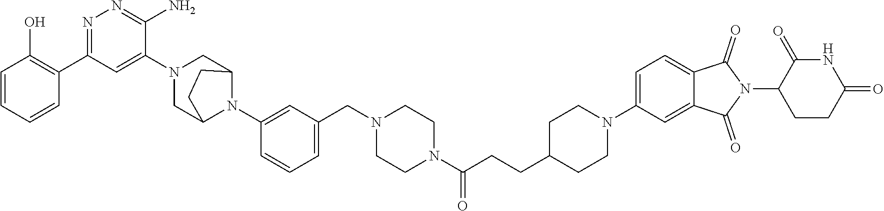 [SMILES: Nc1nnc(-c2ccccc2O)cc1N1CC2CCC(C1)N2c1cccc(CN2CCN(C(=O)CCC3CCN(c4ccc5c(c4)C(=O)N(C4CCC(=O)NC4=O)C5=O)CC3)CC2)c1]

2-[6-Amino-5-[8-[3-(piperazin-1-ylmethyl)phenyl]-3,8-diazabicyclo[3.2.1]octan-3-yl]pyridazin-3-yl]phenol; hydrobromide (35 mg, 63.3 μmol, 1.0 eq; Example 46, Step f) was combined with Ligase 12 (28.8 mg, 69.7 μmol, 1.1 eq), HATU (36.1 mg, 95 μmol, 1.5 eq) and DIPEA (40.9 mg, 55 μL, 317 μmol, 5.0 eq) in DMF (350 μL). The dark orange solution was stirred at ambient temperature for 8 h and then purified by prep-HPLC to afford the title compound (11 mg, 12.7 μmol, 17% yield) as a light yellow solid, 2,2,2-trifluoroacetic acid salt. MS (ESI): 868.4 ([M+H]⁺).

### Example 48

### rac-5-[4-[2-[4-[[3-[3-[3-Amino-6-(2-hydroxyphenyl)pyridazin-4-yl]-3,8-diazabicyclo[3.2.1]octan-8-yl]phenyl]methyl]piperazin-1-yl]-2-oxo-ethyl]-1-piperidyl]-2-(2,6-dioxo-3-piperidyl)isoindoline-1,3-dione

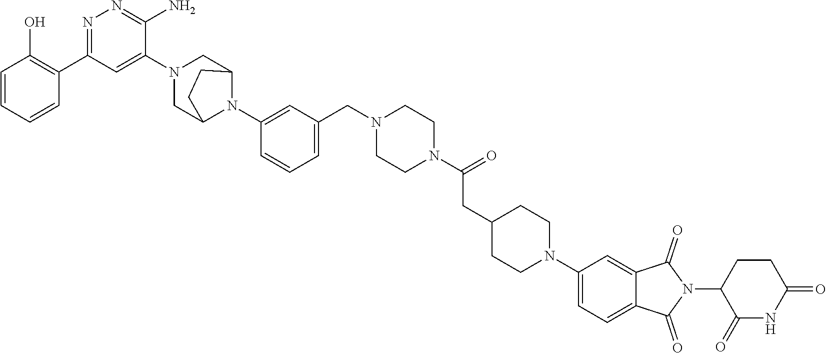 [SMILES: Nc1nnc(-c2ccccc2O)cc1N1CC2CCC(C1)N2c1cccc(CN2CCN(C(=O)CC3CCN(c4ccc5c(c4)C(=O)N(C4CCC(=O)NC4=O)C5=O)CC3)CC2)c1]

2-[6-Amino-5-[8-[3-(piperazin-1-ylmethyl)phenyl]-3,8-diazabicyclo[3.2.1]octan-3-yl]pyridazin-3-yl]phenol; hydrobromide (35 mg, 63.3 μmol, 1.0 eq; Example 46, Step f) was combined with Ligase 24 (27.8 mg, 69.7 μmol, 1.1 eq), HATU (36.1 mg, 95 μmol, 1.5 eq) and DIPEA (40.9 mg, 55 μL, 317 μmol, 5.0 eq) in DMF (350 μL). The dark orange solution was stirred at ambient temperature for 8 h and then purified by prep-HPLC to afford the title compound (14 mg, 16.4 μmol, 23% yield) as a light yellow solid, 2,2,2-trifluoroacetic acid salt. MS (ESI): 851.9 ([M+H]⁺).

### Example 49

### rac-5-[4-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]piperazin-1-yl]-2-(2,6-dioxo-3-piperidyl)isoindoline-1,3-dione

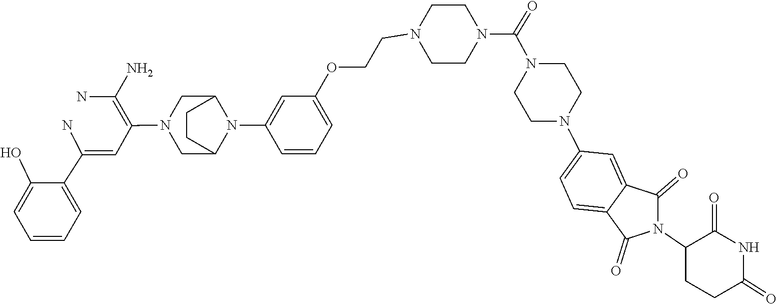

To a stirred solution of (4-nitrophenyl) 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate (34 mg, 51 μmol, 1.0 eq) and Ligase 37 (21.2 mg, 56.1 μmol, 1.1 eq) in DMSO (0.6 mL) at room temperature was added DIPEA (16.5 mg, 22.3 μL, 127 μmol, 2.5 eq). The reaction mixture was stirred at 115° C. for 24 h. The crude material was purified by prep-HPLC to afford the title compound (19 mg, 17.3 μmol, 34% yield) as a yellow solid, bis-(2,2,2-trifluoroacetic acid)salt. MS (ESI): 870.4 ([M+H]⁺).

### Example 50

### rac-3-[4-[1-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]-4-piperidyl]phenoxy]piperidine-2,6-dione

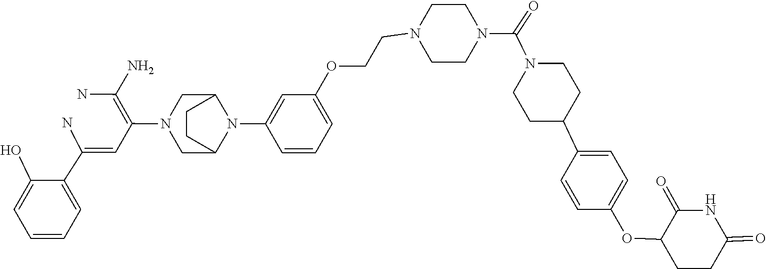

To a stirred solution of (4-nitrophenyl) 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate (34 mg, 51 μmol, 1.0 eq) and Ligase 38 (18.2 mg, 56.1 μmol, 1.1 eq) in DMSO (0.6 mL) at room temperature was added DIPEA (16.5 mg, 22.3 μL, 127 μmol, 2.5 eq). The reaction mixture was stirred at 115° C. for 24 h. The crude material was purified by prep-HPLC and then on silica column (heptane/EtOAc 0-100%) (DCM/MeOH 0-100%)(silica gel, 4 g, 0% to 5% MeOH in DCM). The product was dissolved in acetonitrile/WATER and lyophilised to afford the title compound (12 mg, 14.7 μmol, 29% yield) as an off-white solid. MS (ESI): 816.4 ([M+H]⁺).

### Example 51

### rac-3-[4-[4-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]piperazin-1-yl]phenoxy]piperidine-2,6-dione

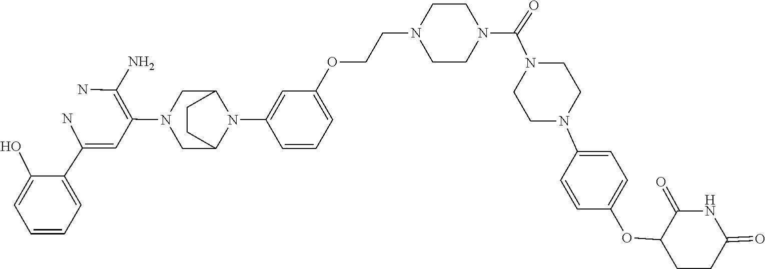

To a stirred solution of (4-nitrophenyl) 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate (34 mg, 51 μmol, 1.0 eq) and Ligase 41 (18.3 mg, 56.1 μmol, 1.1 eq) in DMSO (0.6 mL) at room temperature was added DIPEA (16.5 mg, 22.3 μL, 127 μmol, 2.5 eq). The reaction mixture was stirred at 115° C. for 24 h. The crude material was purified by prep-HPLC. The product was lyophilised to afford the title compound (11 mg, 48.2 μmol, 95% yield) as a light yellow bis-(2,2,2-trifluoroacetic acid)salt. MS (ESI): 817.4 ([M+H]⁺).

### Example 52

### rac-5-[3-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]-3,9-diazaspiro[5.5]undecan-9-yl]-2-(2,6-dioxo-3-piperidyl)isoindoline-1,3-dione

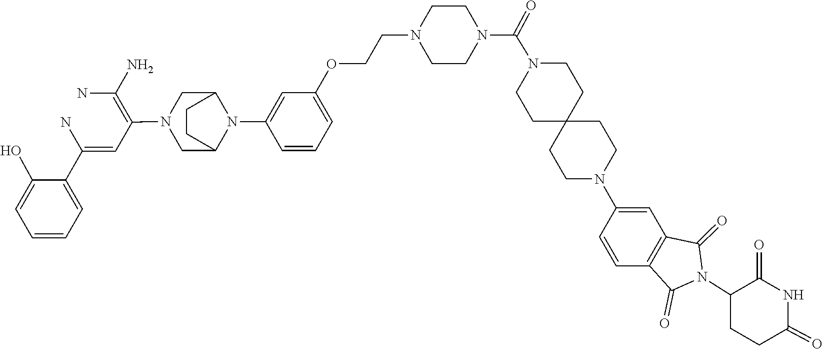

To a stirred solution of (4-nitrophenyl) 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate (34 mg, 51 μmol, 1.0 eq) and Ligase 77 (25.1 mg, 56.1 μmol, 1.1 eq) in DMSO (0.6 mL) at room temperature was added DIPEA (16.5 mg, 22.3 μL, 127 μmol, 2.5 eq). The reaction mixture was stirred at 115° C. for 16 h. The reaction mixture was poured into EtOAc/THF (1:2) and washed sequentially with water and with brine. The organic layer was dried over Na₂SO₄ and concentrated in vacuo. The crude material was purified on silica column (DCM/MeOH 0-5%) to afford the title compound (12 mg, 12.8 μmol, 25% yield) as a yellow solid. MS (ESI): 938.5 ([M+H]⁺).

### Example 53

### 4-[3-amino-6-(2-hydroxyphenyl)pyridazin-4-yl]-N-[1-[2-[1-[2-(2,6-dioxo-3-piperidyl)-1,3-dioxo-isoindolin-5-yl]-4-piperidyl]acetyl]-4-piperidyl]-1-phenyl-piperazine-2-carboxamide

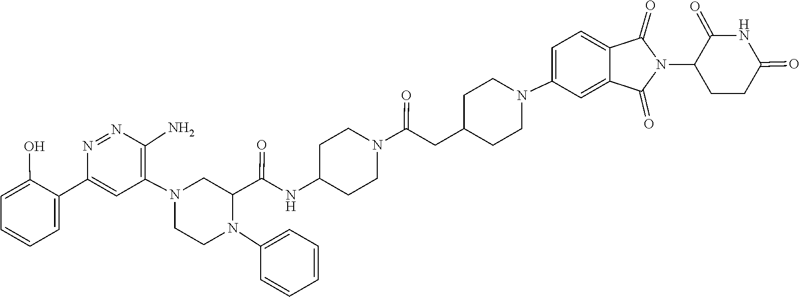 [SMILES: Nc1nnc(-c2ccccc2O)cc1N1CCN(c2ccccc2)C(C(=O)NC2CCN(C(=O)CC3CCN(c4ccc5c(c4)C(=O)N(C4CCC(=O)NC4=O)C5=O)CC3)CC2)C1]

### a) tert-butyl 3-[(1-benzyloxycarbonyl-4-piperidyl)carbamoyl]-4-phenyl-piperazine-1-carboxylate

To a solution of 4-(tert-butoxycarbonyl)-1-phenylpiperazine-2-carboxylic acid (750 mg, 1.84 mmol, 1.0 eq), HATU (1.05 g, 2.75 mmol, 1.5 eq) and DIPEA (1.42 g, 1.92 mL, 11 mmol, 6.0 eq) in DMF (6 mL) was added benzyl 4-aminopiperidine-1-carboxylate (645 mg, 2.75 mmol, 1.5 eq). The reaction mixture was stirred at room temperature for 2 h. The reaction mixture was concentrated in vacuo. The crude material was purified on silica column (heptane/EtOAc 0-100%) to afford the title compound (680 mg, 1.30 mmol, 71% yield) as an off-white foam. MS (ESI): 523.4 ([M+H]⁺).

### b) benzyl 4-[(1-phenylpiperazine-2-carbonyl)amino]piperidine-1-carboxylate

To a cooled (0° C.) solution of tert-butyl 3-((1-((benzyloxy)carbonyl)piperidin-4-yl)carbamoyl)-4-phenylpiperazine-1-carboxylate (680 mg, 1.3 mmol, 1.0 eq) in DCM (6 mL) was added 4M HCl in dioxane (1.3 mL, 5.2 mmol, 4.0 eq). The reaction mixture was allowed to reach room temperature and stirred for 20 h. The reaction mixture was concentrated in vacuo to afford the title compound (550 mg, 1.3 mmol, 99% yield) as a white hydrochloride salt. MS (ESI): 423.3 ([M+H]⁺).

### c) benzyl 4-[[4-(3-amino-6-chloro-pyridazin-4-yl)-1-phenyl-piperazine-2-carbonyl]amino]piperidine-1-carboxylate

To a stirred solution of 4-bromo-6-chloropyridazin-3-amine (350 mg, 1.68 mmol, 1.0 eq) and benzyl 4-(1-phenylpiperazine-2-carboxamido)piperidine-1-carboxylate hydrochloride (730 mg, 1.68 mmol, 1.0 eq) in DMA (6 mL) was added potassium carbonate (580 mg, 4.2 mmol, 2.5 eq). The reaction mixture was heated to 110° C. and stirred for 16 h. The reaction mixture was poured in WATER and extracted with EtOAc. The organic layers were combined, washed with saturated NaHCO₃, WATER and brine. The organic layers were dried over Na₂SO₄ and concentrated in vacuo. The crude material was purified on silica column (heptane/EtOAc 0-100%) to afford the title compound (275 mg, 501 μmol, 30% yield) as a brown oil. MS (ESI): 550.3 ([M+H]⁺).

### d) benzyl 4-[[4-[3-amino-6-(2-hydroxyphenyl)pyridazin-4-yl]-1-phenyl-piperazine-2-carbonyl]amino]piperidine-1-carboxylate

A suspension of benzyl 4-(4-(3-amino-6-chloropyridazin-4-yl)-1-phenylpiperazine-2-carboxamido)piperidine-1-carboxylate (275 mg, 500 μmol, 1.0 eq), (2-hydroxyphenyl)boronic acid (138 mg, 1000 μmol, 2.0 eq), potassium carbonate (207 mg, 1.5 mmol, 3.0 eq) and RuPhos Pd G3 (20.9 mg, 25 μmol, 0.05 eq) in a mixture of degassed dioxane (6 mL) and WATER (0.6 mL) was stirred at 120° C. for 16 h under argon. The reaction mixture was poured in saturated NaHCO₃ and extracted with EtOAc. The organic layers were combined, washed with WATER and brine. The organic layers were dried over Na₂SO₄ and concentrated in vacuo. The crude material was purified on silica column (DCM/MeOH 0-5%) to afford the title compound (170 mg, 280 μmol, 56% yield) as a brown foam. MS (ESI): 608.4 ([M+H]⁺).

### e) 4-[3-amino-6-(2-hydroxyphenyl)pyridazin-4-yl]-1-phenyl-N-(4-piperidyl)piperazine-2-carboxamide

To a solution of benzyl 4-(4-(3-amino-6-(2-hydroxyphenyl)pyridazin-4-yl)-1-phenylpiperazine-2-carboxamido)piperidine-1-carboxylate (140 mg, 230 μmol, 1.0 eq) in methanol (4 mL) was added 10% palladium on charcoal (24.5 mg, 23 μmol, 0.1 eq). The reaction mixture was vigorously stirred at room temperature for 5 h under H₂ (baloon). The catalyst was collected by filtration, washing with methanol. The filtrate was concentrated and the crude material was purified on silica column (DCM/MeOH 0-5%) to afford the title compound (85 mg, 180 μmol, 78% yield) as a light brown oil. MS (ESI): 474.4 ([M+H]⁺).

### f) 4-[3-amino-6-(2-hydroxyphenyl)pyridazin-4-yl]-N-[1-[2-[1-[2-(2,6-dioxo-3-piperidyl)-1,3-dioxo-isoindolin-5-yl]-4-piperidyl]acetyl]-4-piperidyl]-1-phenyl-piperazine-2-carboxamide

Ligase 24 (12.9 mg, 32.4 μmol, 1.1 eq) was dissolved in dry DMF (200 μL). DIPEA (9.5 mg, 12.8 μL, 73.5 μmol, 2.5 eq) and HATU (13.4 mg, 35.3 μmol, 1.2 eq) were added and the mixture was stirred at room temperature for 10 min. 4-(3-amino-6-(2-hydroxyphenyl)pyridazin-4-yl)-1-phenyl-N-(piperidin-4-yl)piperazine-2-carboxamide, hydrochloride salt (15 mg, 29.4 μmol, 1.0 eq) was added and the mixture was stirred at room temperature for 2 h. The reaction mixture was purified directly by prep-HPLC to afford the title compound (18 mg, 17.6 μmol, 60% yield) as a yellow solid, (2,2,2-trifluoroacetate) salt. MS (ESI): 855.7 ([M+H]⁺).

### Example 54

### 4-[3-amino-6-(2-hydroxyphenyl)pyridazin-4-yl]-N-[1-[3-[1-[2-(2,6-dioxo-3-piperidyl)-1,3-dioxo-isoindolin-5-yl]-4-piperidyl]propanoyl]-4-piperidyl]-1-phenyl-piperazine-2-carboxamide

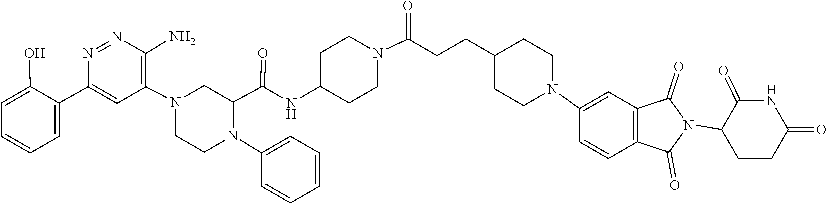 [SMILES: Nc1nnc(-c2ccccc2O)cc1N1CCN(c2ccccc2)C(C(=O)NC2CCN(C(=O)CCC3CCN(c4ccc5c(c4)C(=O)N(C4CCC(=O)NC4=O)C5=O)CC3)CC2)C1]

The title compound was prepared in analogy to example 53 step f using Ligase 12 as a yellow solid (19 mg, 19.3 μmol, 62% yield), (2,2,2-trifluoroacetate) salt. MS (ESI): 869.6 ([M+H]⁺).

### Example 55

### rac-5-[4-[2-[4-[[3-[3-[3-amino-6-(2-hydroxyphenyl)pyridazin-4-yl]-3,8-diazabicyclo[3.2.1]octan-8-yl]phenyl]methyl]piperazin-1-yl]ethoxy]-1-piperidyl]-2-(2,6-dioxo-3-piperidyl)isoindoline-1,3-dione

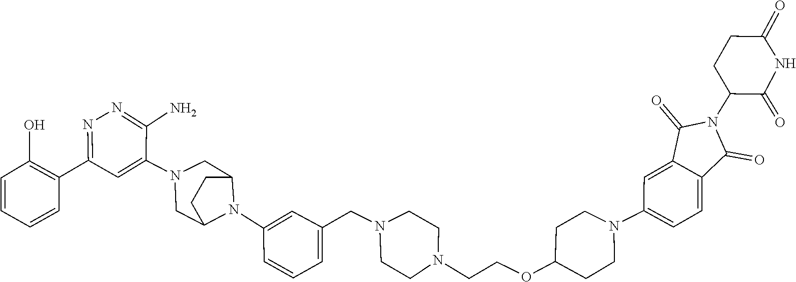 [SMILES: Nc1nnc(-c2ccccc2O)cc1N1CC2CCC(C1)N2c1cccc(CN2CCN(CCOC3CCN(c4ccc5c(c4)C(=O)N(C4CCC(=O)NC4=O)C5=O)CC3)CC2)c1]

2-[6-Amino-5-[8-[3-(piperazin-1-ylmethyl)phenyl]-3,8-diazabicyclo[3.2.1]octan-3-yl]pyridazin-3-yl]phenol; hydrobromide (35 mg, 63.3 μmol, 1.0 eq; Example 46, Step f) was combined with Ligase 26 (32.4 mg, 69.7 μmol, 1.1 eq) and DIPEA (40.9 mg, 55 μL, 317 μmol, 5.0 eq) in DMF (350 μL). The dark orange solution was stirred at 60° C. for 16 h and then purified by prep-HPLC to afford the title compound (26 mg, 30.4 μmol, 41% yield) as a light yellow solid, 2,2,2-trifluoroacetic acid salt. MS (ESI): 854.8 ([M+H]⁺)

### Example 56

### rac-5-[4-[3-[4-[[3-[3-[3-Amino-6-(2-hydroxyphenyl)pyridazin-4-yl]-3,8-diazabicyclo[3.2.1]octan-8-yl]phenyl]methyl]piperazin-1-yl]propoxy]-1-piperidyl]-2-(2,6-dioxo-3-piperidyl)isoindoline-1,3-dione

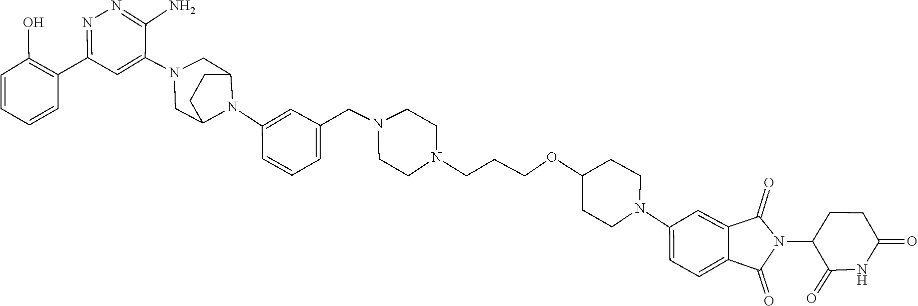 [SMILES: Nc1nnc(-c2ccccc2O)cc1N1CC2CCC(C1)N2c1cccc(CN2CCN(CCCOC3CCN(c4ccc5c(c4)C(=O)N(C4CCC(=O)NC4=O)C5=O)CC3)CC2)c1]

2-[6-Amino-5-[8-[3-(piperazin-1-ylmethyl)phenyl]-3,8-diazabicyclo[3.2.1]octan-3-yl]pyridazin-3-yl]phenol; hydrobromide (35 mg, 63.3 μmol, 1.0 eq; Example 46, Step f) was combined with Ligase 27 (33.3 mg, 69.7 μmol, 1.1 eq) and DIPEA (40.9 mg, 55 μL, 317 μmol, 5.0 eq) in DMF (350 μL). The dark orange solution was stirred at 60° C. for 16 h and then purified by prep-HPLC to afford the title compound (25 mg, 28.8 μmol, 40% yield) as a light yellow solid, 2,2,2-trifluoroacetic acid salt. MS (ESI): 867.9 ([M+H]⁺)

### Example 57

### 5-[4-[[4-[2-[3-[8-[3-amino-6-(5-fluoro-2-hydroxy-phenyl)pyridazin-4-yl]-3,8-diazabicyclo[3.2.1]octan-3-yl]phenoxy]ethyl]piperazin-1-yl]methyl]-1-piperidyl]-2-(2,6-dioxo-3-piperidyl)isoindoline-1,3-dione

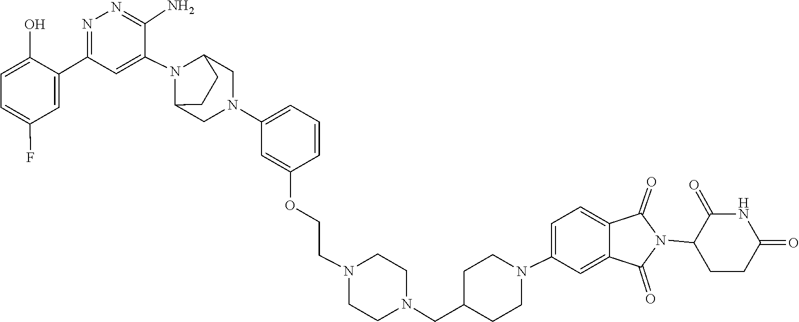 [SMILES: Nc1nnc(-c2cc(F)ccc2O)cc1N1C2CCC1CN(c1cccc(OCCN3CCN(CC4CCN(c5ccc6c(c5)C(=O)N(C5CCC(=O)NC5=O)C6=O)CC4)CC3)c1)C2]

### a) tert-butyl 3-(3-benzyloxyphenyl)-3,8-diazabicyclo[3.2.1]octane-8-carboxylate

To a suspension of 1-(benzyloxy)-3-bromobenzene (5 g, 19 mmol, 1.0 eq) and tert-butyl 3,8-diazabicyclo[3.2.1]octane-8-carboxylate (4.24 g, 20 mmol, 1.05 eq) in t-BuOH (30 mL) was added K₂CO₃ (5.25 g, 38 mmol, 2.0 eq). The reaction was degassed with argon for 5 min. RuPhos Pd G3 (1.34 g, 1.6 mmol, 0.0843 eq) was added. The reaction mixture was stirred at 120° C. overnight. The catalyst was removed by filtration and washed with EtOAc. The residue was poured into EtOAc/THF 2:1 and washed with water and brine. The organic layer was dried over Na₂SO₄ and concentrated in vacuo. The crude material was purified on silica column (heptane/EtOAc 0-18) to afford the title compound (3.4 g, 8.62 mmol, 45% yield) as a yellow oil. MS (ESI): 395.2358 ([M+H]⁺).

### b) tert-butyl 3-(3-hydroxyphenyl)-3,8-diazabicyclo[3.2.1]octane-8-carboxylate

To a solution of tert-butyl 3-(3-benzyloxyphenyl)-3,8-diazabicyclo[3.2.1]octane-8-carboxylate (3.4 g, 8.62 mmol, 1.0 eq) in methanol (200 mL) was added ammonium formate (10.9 g, 172 mmol, 20 eq). The reaction mixture was degassed with argon for 10 min. Pd—C 10% (917 mg, 862 μmol, 0.1 eq) was added. The reaction mixture was stirred for 2 h at 70° C. The reaction mixture was filtered, concentrated in vacuo. The reaction mixture was poured into AcOEt/THF 1:1 and washed with WATER and brine. The organic layer was dried over Na₂SO₄ and concentrated in vacuo to afford the title compound (2.57 g, 8.44 mmol, 98% yield) as an off-white solid. MS (ESI): 305.1893 ([M+H]⁺).

### c) tert-butyl 3-[3-[2-(4-benzyloxycarbonylpiperazin-1-yl)ethoxy]phenyl]-3,8-diazabicyclo[3.2.1]octane-8-carboxylate

To a solution of tert-butyl 3-(3-hydroxyphenyl)-3,8-diazabicyclo[3.2.1]octane-8-carboxylate (2.56 g, 8.41 mmol, 1.0 eq) and benzyl 4-(2-hydroxyethyl)piperazine-1-carboxylate (2.38 g, 9 mmol, 1.07 eq) in THF (20 mL) was added benzyl 4-(2-hydroxyethyl)piperazine-1-carboxylate (2.38 g, 9 mmol, 1.07 eq). The reaction mixture was stirred for 2 h overnight at 70° C. 2-(Trimethylphosphoranylidene)acetonitrile 0.5M in THF (20 mL, 10 mmol, 1.19 eq) was added. The reaction mixture was stirred for 2 h at 70° C. The reaction mixture was poured into THF/AcOEt 3:1 and washed with WATER/brine. The organic layer was dried over Na₂SO₄ and concentrated in vacuo. The crude material was purified on silica column (heptane/EtOAc 0-100%) (DCM/MeOH 0-100%) to afford the title compound (3.66 g, 6.65 mmol, 79% yield) as a brown oil. MS (ESI): 551.3228 ([M+H]⁺).

### d) benzyl 4-[2-[3-(3,8-diazabicyclo[3.2.1]octan-3-yl)phenoxy]ethyl]piperazine-1-carboxylate

To a solution of tert-butyl 3-[3-[2-(4-benzyloxycarbonylpiperazin-1-yl)ethoxy]phenyl]-3,8-diazabicyclo[3.2.1]octane-8-carboxylate (3.6 g, 6.54 mmol, 1.0 eq) in CH₂Cl₂ (25 mL) was added TFA (14.8 g, 10 mL, 130 mmol, 1.0 eq). The reaction mixture was stirred for 2 h at room temperature. The reaction mixture was concentrated in vacuo, poured into AcOEt/THF 1:2 and washed with NaOH 1N/brine 1:1. The organic layer was dried over Na₂SO₄ and concentrated in vacuo to afford the title compound (3.77 g, 8.37 mmol, 128% yield) as a brown oil, trifluoro acetic acid salt. MS (ESI): 451.2742 ([M+H]⁺).

### e) benzyl 4-[2-[3-[8-(3-amino-6-chloro-pyridazin-4-yl)-3,8-diazabicyclo[3.2.1]octan-3-yl]phenoxy]ethyl]piperazine-1-carboxylate

To a solution of benzyl 4-[2-[3-(3,8-diazabicyclo[3.2.1]octan-3-yl)phenoxy]ethyl]piperazine-1-carboxylate (3.773 g, 6.78 mmol, 1.0 eq) and 4-bromo-6-chloropyridazin-3-amine (1.7 g, 8.14 mmol, 1.2 eq) in DMSO (12 mL) was added K₂CO₃ (4.69 g, 33.9 mmol, 5.0 eq). The reaction mixture was stirred for 16 h at 110° C. The reaction mixture was poured into THF/AcOEt 2:1 and washed with WATER/brine. The organic layer was dried over Na₂SO₄ and concentrated in vacuo. The crude material was purified on silica column (DCM/MeOH 0-5%) to afford the title compound (2.74 g, 4.55 mmol, 67% yield) as a brown foam. MS (ESI): 578.2665 ([M+H]⁺).

### f) benzyl 4-[2-[3-[8-[3-amino-6-(5-fluoro-2-hydroxy-phenyl)pyridazin-4-yl]-3,8-diazabicyclo[3.2.1]octan-3-yl]phenoxy]ethyl]piperazine-1-carboxylate

Benzyl 4-[2-[3-[8-(3-amino-6-chloro-pyridazin-4-yl)-3,8-diazabicyclo[3.2.1]octan-3-yl]phenoxy]ethyl]piperazine-1-carboxylate (400 mg, 692 μmol, 1.0 eq) was stirred with (5-fluoro-2-hydroxyphenyl)boronic acid (216 mg, 1.38 mmol, 2.0 eq) and potassium carbonate (239 mg, 1.73 mmol, 2.5 eq) in dioxane (3 mL), DMA (600 μL) and water (200 μL). Argon was bubbled trough the reaction for 2 min. Then RuPhos Pd G3 (28.9 mg, 34.6 μmol, 0.05 eq) was added. The reaction mixture was stirred overnight at 130° C. in a closed vessel. The crude material was purified on silica column (DCM/MeOH 0-10%) as eluent to afford the title compound (231 mg, 339 μmol, 49% yield) as a dark brown foam. MS (ESI): 654.3228 ([M+H]⁺).

### g) 2-[6-amino-5-[3-[3-(2-piperazin-1-ylethoxy)phenyl]-3,8-diazabicyclo[3.2.1]octan-8-yl]pyridazin-3-yl]-4-fluoro-phenol

Benzyl 4-[2-[3-[8-[3-amino-6-(5-fluoro-2-hydroxy-phenyl)pyridazin-4-yl]-3,8-diazabicyclo[3.2.1]octan-3-yl]phenoxy]ethyl]piperazine-1-carboxylate (225 mg, 344 μmol, 1.0 eq) was stirred with palladium (36.6 mg, 34.4 μmol, 0.1 eq) in methanol (10 mL) and THF (2 mL) under a hydrogen atmosphere at room temperature overnight. The catalyst was filtered off and the solvent was evaporated under reduced pressure, then dried under high vacuum to afford the title compound (166 mg, 300 μmol, 87.3% yield) as an off-white solid. MS (ESI): 518.2676 ([M−H]⁻).

### h) 5-[4-[[4-[2-[3-[8-[3-amino-6-(5-fluoro-2-hydroxy-phenyl)pyridazin-4-yl]-3,8-diazabicyclo[3.2.1]octan-3-yl]phenoxy]ethyl]piperazin-1-yl]methyl]-1-piperidyl]-2-(2,6-dioxo-3-piperidyl)isoindoline-1,3-dione

2-[6-amino-5-[3-[3-(2-piperazin-1-ylethoxy)phenyl]-3,8-diazabicyclo[3.2.1]octan-8-yl]pyridazin-3-yl]-4-fluoro-phenol (35 mg, 67.4 μmol, 1.0 eq) was stirred with Ligase 33 (29.3 mg, 67.4 μmol, 1.0 eq) and triethylamine (13.6 mg, 18.8 μL, 135 μmol, 2.0 eq) in acetonitrile (250 μL) and DMF (200 μL) in a closed vessel at 110° C. for 1 h. The crude mixture was purified by prep-HPLC to afford the title compound (29 mg, 25.8 μmol, 38.3% yield) as a yellow bis-(2,2,2-trifluoroacetic acid) salt. MS (ESI): 873.4212 ([M+H]⁺).

### Example 58

### rac-4-[[3-[4-[[3-[3-[3-amino-6-(2-hydroxyphenyl)pyridazin-4-yl]-3,8-diazabicyclo[3.2.1]octan-8-yl]phenyl]methyl]-1-piperidyl]-3-oxo-propyl]amino]-2-(2,6-dioxo-3-piperidyl)isoindoline-1,3-dione

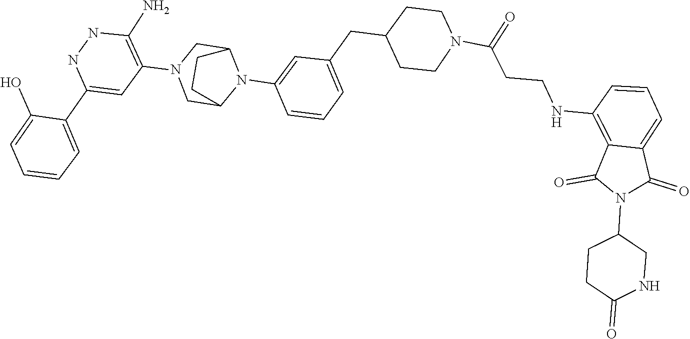 [SMILES: NC1=C(N2CC3CCC(C2)N3c2cccc(CC3CCN(C(=O)CCNc4cccc5c4C(=O)N(C4CCC(=O)NC4)C5=O)CC3)c2)C=C(c2ccccc2O)NN1]

To a stirred suspension of 2-[6-amino-5-[8-[3-(4-piperidylmethyl)phenyl]-3,8-diazabicyclo[3.2.1]octan-3-yl]pyridazin-3-yl]phenol (25 mg, 53.1 μmol, 1.0 eq), Ligase 43 (20.2 mg, 58.4 μmol, 1.1 eq) and DIPEA (34.3 mg, 46.4 μL, 266 μmol, 5.0 eq) in DMF (0.5 mL) at room temperature was added HATU (40.4 mg, 106 μmol, 2.0 eq). The reaction mixture was stirred at room temperature for 1 h. The crude material was purified by prep-HPLC and the product was lyophilised to afford the title compound (12 mg, 13.2 μmol, 25% yield) as a yellow solid, 2,2,2-trifluoroacetate salt. MS (ESI): 798.4 ([M+H]⁺).

### Example 59

### rac-5-[4-[3-[4-[[3-[3-[3-amino-6-(2-hydroxyphenyl)pyridazin-4-yl]-3,8-diazabicyclo[3.2.1]octan-8-yl]phenyl]methyl]-1-piperidyl]-3-oxo-propyl]-1-piperidyl]-2-(2,6-dioxo-3-piperidyl)isoindoline-1,3-dione

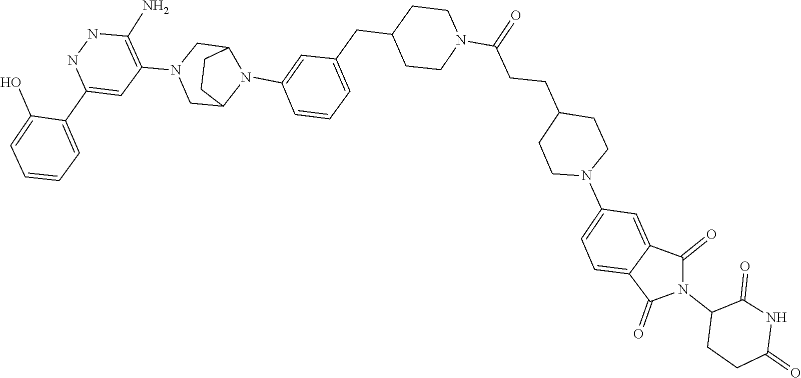 [SMILES: NC1=C(N2CC3CCC(C2)N3c2cccc(CC3CCN(C(=O)CCC4CCN(c5ccc6c(c5)C(=O)N(C5CCC(=O)NC5=O)C6=O)CC4)CC3)c2)C=C(c2ccccc2O)NN1]

To a stirred solution of 2-[6-amino-5-[8-[3-(4-piperidylmethyl)phenyl]-3,8-diazabicyclo[3.2.1]octan-3-yl]pyridazin-3-yl]phenol (25 mg, 53.1 μmol, 1.0 eq), Ligase 12 (24.2 mg, 58.4 μmol, 1.1 eq) and DIPEA (34.3 mg, 46.4 μL, 266 μmol, 5.0 eq) in DMF (0.5 mL) at room temperature was added HATU (40.4 mg, 106 μmol, 2.0 eq). The reaction mixture was stirred at room temperature for 1 h. The crude material was purified by prep-HPLC and the product was lyophilised to afford the title compound (15 mg, 15.3 μmol, 29% yield) as a yellow solid, 2,2,2-trifluoroacetate salt. MS (ESI): 866.4 ([M+H]⁺).

### Example 60

### rac-5-[3-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]azetidin-1-yl]-2-(2,6-dioxo-3-piperidyl)isoindoline-1,3-dione

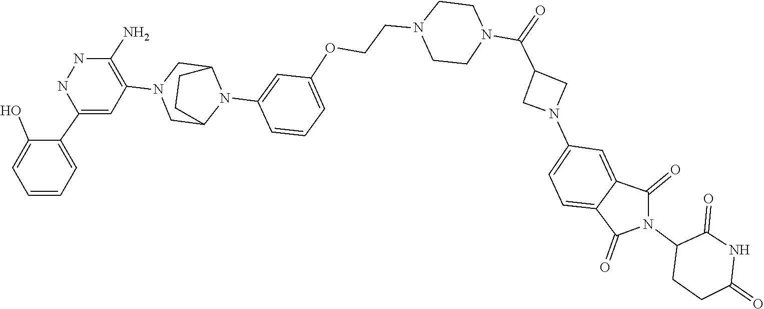

To a crude solution of Ligase 44 (109 μmol) in DMSO were added 2-[6-amino-5-[8-[3-(2-piperazin-1-ylethoxy)phenyl]-3,8-diazabicyclo[3.2.1]octan-3-yl]pyridazin-3-yl]phenol (60.2 mg, 120 μmol, 1.1 eq), DIPEA (14.1 mg, 19.1 μL, 109 μmol, 1.0 eq) and HATU (83 mg, 218 umol, 2.0 eq) at room temperature. The reaction mixture was stirred at room temperature for 2 h, poured into EtOAc/THF (1:1) and washed with water and brine. The crude material was purified on silica column (DCM/MeOH 0-10%) to afford the title compound (43 mg, 51.1 μmol, 47% yield) as a yellow solid. MS (ESI): 841.4 ([M+H]⁺).

### Example 61

### rac-5-[6-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]-2-azaspiro[3.3]heptan-2-yl]-2-(2,6-dioxo-3-piperidyl)isoindoline-1,3-dione

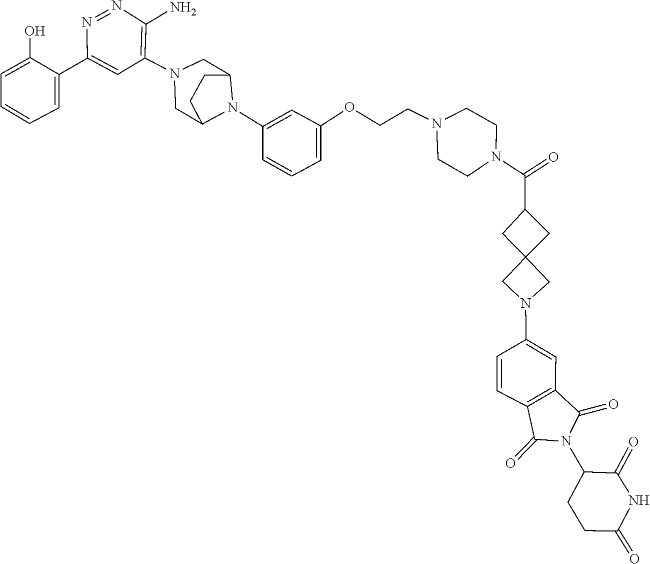 [SMILES: Nc1nnc(-c2ccccc2O)cc1N1CC2CCC(C1)N2c1cccc(OCCN2CCN(C(=O)C3CC4(C3)CN(c3ccc5c(c3)C(=O)N(C3CCC(=O)NC3=O)C5=O)C4)CC2)c1]

### a) tert-butyl 6-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]-2-azaspiro[3.3]heptane-2-carboxylate

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (125 mg, 249 μmol, 1.0 eq), 2-(tert-butoxycarbonyl)-2-azaspiro[3.3]heptane-6-carboxylic acid (62 mg, 249 μmol, 1.0 eq) and DIPEA (96.6 mg, 131 μL, 748 μmol, 3.0 eq) in DMF at room temperature was added HATU (208 mg, 548 μmol, 2.2 eq). The reaction mixture was stirred for 3 h. The reaction mixture was partitioned between EtOAc/THF (1:1) and water. The layers were separated. The aqueous layer was extracted with EtOAc/THF. The combined organic layers were washed with brine, dried over anhydrous sodium sulfate, and concentrated in vacuo. The crude material was purified on silica column (DCM/MeOH 0-10%) and on amine modified silica column (heptane/EtOAc 0-100%) to afford the title compound (69.7 mg, 96 μmol, 39% yield) as a white solid. MS (ESI): 725.7 ([M+H]⁺).

### b) [4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-(2-azaspiro[3.3]heptan-6-yl)methanone

To a stirred solution of tert-butyl 6-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]-2-azaspiro[3.3]heptane-2-carboxylate (69 mg, 95.2 μmol, 1.0 eq) in DCM (2 mL) at room temperature was added TFA (1.48 g, 1 mL, 13 mmol, 136 eq). The reaction mixture was stirred for 2 h. The reaction mixture was quenched at room temperature with saturated bicarbonate solution and stirred for 10 min. The mixture was partitioned between EtOAc/THF (1:1) and saturated aqueous sodium bicarbonate solution. The layers were separated. The aqueous layer was extracted with THF. The combined organic layers were dried over anhydrous sodium sulfate and concentrated in vacuo to afford the title compound (821 mg, 92 μmol, 97% yield) as an off-white solid. MS (ESI): 625.5 ([M+H]⁺).

### c) rac-5-[6-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]-2-azaspiro[3.3]heptan-2-yl]-2-(2,6-dioxo-3-piperidyl)isoindoline-1,3-dione

A solution of [4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-(2-azaspiro[3.3]heptan-6-yl)methanone (821 mg, 92 μmol, 1.0 eq) and 2-(2,6-dioxo-3-piperidyl)-5-fluoro-isoindoline-1,3-dione (25.4 mg, 92 μmol, 1.0 eq) in DMSO (1 mL) was stirred at 95° C. for 25 h. The reaction mixture was cooled to room temperature and then partitioned between EtOAc/THF and water. The aqueous layer was extracted with EtOAc/THF (1:1). The combined organic layers were dried over anhydrous sodium sulfate and concentrated in vacuo. The crude material was purified on silica column (DCM/MeOH 0-8%) to afford the title compound (10.2 mg, 11.6 μmol, 13% yield) as a yellow solid. MS (ESI): 881.4 ([M+H]⁺).

### Example 62

### 2-(2,6-dioxo-3-piperidyl)-4-[[1-[9-oxo-9-[4-[4-[rac-(3R)-3-[3-amino-6-(2-hydroxyphenyl)pyridazin-4-yl]oxy-1-piperidyl]phenyl]piperazin-1-yl]nonyl]triazol-4-yl]methylamino]isoindoline-1,3-dione

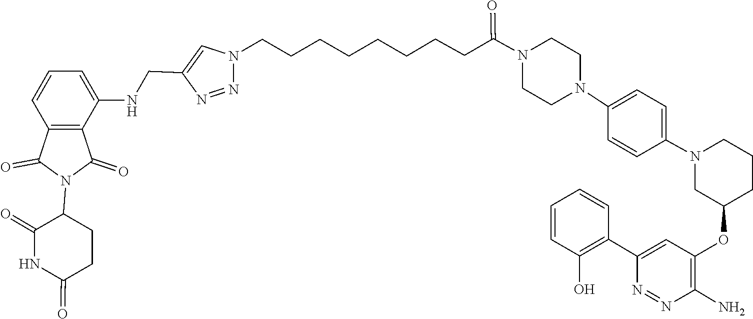 [SMILES: Nc1nnc(-c2ccccc2O)cc1O[C@@H]1CCCN(c2ccc(N3CCN(C(=O)CCCCCCCCn4cc(CNc5cccc6c5C(=O)N(C5CCC(=O)NC5=O)C6=O)nn4)CC3)cc2)C1]

To a solution of 2-[6-amino-5-[[(3S)-1-(4-piperazin-1-ylphenyl)-3-piperidyl]oxy]pyridazin-3-yl]phenol (40.0 mg, 71.48 μmol, TFA salt) and Ligase 3 (36 mg, 71.48 μmol) in DMF (0.8 mL) was added HATU (40 mg, 107 μmol) followed by DIPEA (46 mg, 357.42 μmol, 62 μL) and the reaction mixture was stirred at room temperature for 16 h. The reaction mixture was diluted with water and extracted with EtOAc. The volatiles were removed. The crude residue was purified by prep-HPLC to afford the title compound (31.5 mg, 32.18 μmol, 45% yield) as a light green solid. MS (ESI). 939.4 ([M+H]⁺).

### Example 63

### 4-[[1-[9-[4-[4-[(3R)-3-[3-amino-6-(2-hydroxyphenyl)pyridazin-4-yl]oxy-1-piperidyl]phenyl]piperazin-1-yl]nonyl]triazol-4-yl]methylamino]-2-(2,6-dioxo-3-piperidyl)isoindoline-1,3-dione

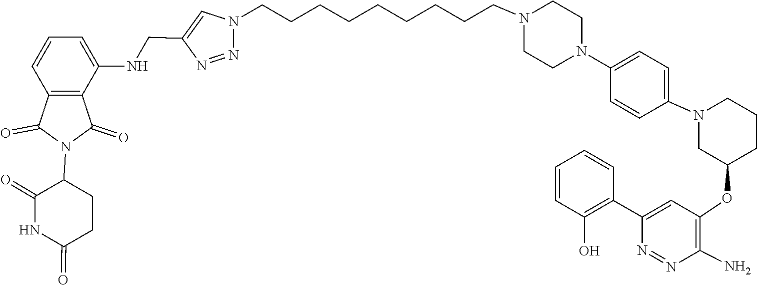 [SMILES: Nc1nnc(-c2ccccc2O)cc1O[C@@H]1CCCN(c2ccc(N3CCN(CCCCCCCCCn4cc(CNc5cccc6c5C(=O)N(C5CCC(=O)NC5=O)C6=O)nn4)CC3)cc2)C1]

To a solution of 2-[6-amino-5-[[(3S)-1-(4-piperazin-1-ylphenyl)-3-piperidyl]oxy]pyridazin-3-yl]phenol (40.0 mg, 71.48 μmol, TFA salt) and Ligase 28 (42 mg, 85.78 μmol) in THF (2.0 mL) was added trimethyl(phenyl)silane (9 mg, 85 μmol) followed by dibutyltin dichloride (21 mg, 71.48 μmol) and the reaction mixture was heated to 80° C. for 16 h. The reaction mixture was cooled to room temperature, diluted with water and extracted with EtOAc. The organic layer was washed with water, brine solution, dried over anhydrous sodium sulfate, filtered and concentrated under reduced pressure. The crude residue was purified by prep-HPLC to afford the title compound (7.6 mg, 7.7 μmol, 10% yield) as an off white solid. MS (ESI): 925.3 ([M+H]⁺).

### Example 64

### 4-[[1-[9-[4-[4-[3-[3-amino-6-(5-fluoro-2-hydroxy-phenyl)pyridazin-4-yl]oxy-1-piperidyl]phenyl]piperazin-1-yl]-9-oxo-nonyl]triazol-4-yl]methylamino]-2-(2,6-dioxo-3-piperidyl)isoindoline-1,3-dione

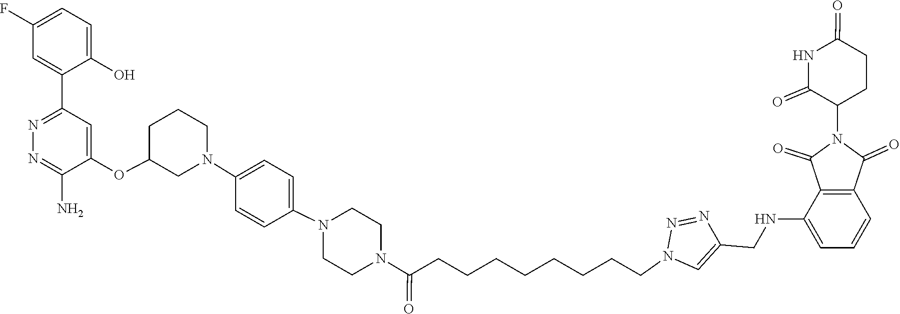 [SMILES: Nc1nnc(-c2cc(F)ccc2O)cc1OC1CCCN(c2ccc(N3CCN(C(=O)CCCCCCCCn4cc(CNc5cccc6c5C(=O)N(C5CCC(=O)NC5=O)C6=O)nn4)CC3)cc2)C1]

The title compound (4.66 mg, 4.56 μmol, 7% yield) was prepared in analogy to example 3 step e using Ligase 3 as a light yellow solid, trifluoroacetic acid salt. (MS (ESI): 957.9 ([M+H]⁺).

### Example 65

### 4-[[1-[9-[4-[4-[3-[3-amino-6-(2-hydroxyphenyl)pyridazin-4-yl]oxy-1-piperidyl]phenyl]piperazin-1-yl]nonyl]triazol-4-yl]methoxy]-2-(2,6-dioxo-3-piperidyl)isoindoline-1,3-dione

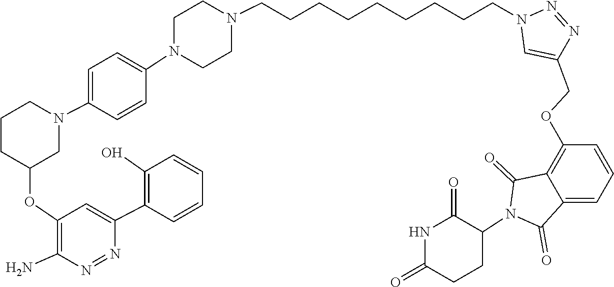 [SMILES: Nc1nnc(-c2ccccc2O)cc1OC1CCCN(c2ccc(N3CCN(CCCCCCCCCn4cc(COc5cccc6c5C(=O)N(C5CCC(=O)NC5=O)C6=O)nn4)CC3)cc2)C1]

To a solution of 2-[6-amino-5-[[1-(4-piperazin-1-ylphenyl)-3-piperidyl]oxy]pyridazin-3-yl]phenol (50 mg, 89 μmol, TFA salt) and Ligase 45 (44.28 mg, 89 μmol) in THF (3 mL) was added trimethyl(phenyl)silane (20 mg, 178 μmol) followed by dibutyltin dichloride (54 mg, 178 μmol) and the reaction mixture was heated to 80° C. for 16 h. The reaction mixture was cooled to room temperature, diluted with water and extracted with EtOAc. The organic layer was washed with water, brine solution, dried over anhydrous sodium sulfate, filtered and concentrated under reduced pressure. The crude residue was purified by prep-HPLC to afford the title compound (5.0 mg, 4.95 μmol, 5% yield) as an off-white solid. MS (ESI): 926.3 ([M+H]⁺).

### Example 66

### 2-(2,6-dioxo-3-piperidyl)-4-[[1-[9-[4-[4-[rac-(3S)-3-[3-amino-6-(2-hydroxyphenyl)pyridazin-4-yl]oxy-1-piperidyl]phenyl]piperazin-1-yl]nonyl]triazol-4-yl]methylamino]isoindoline-1,3-dione

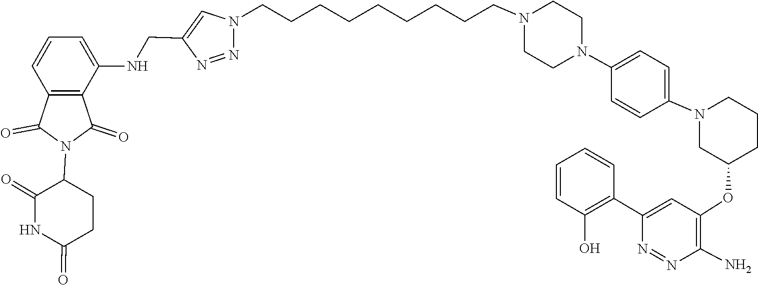 [SMILES: Nc1nnc(-c2ccccc2O)cc1O[C@H]1CCCN(c2ccc(N3CCN(CCCCCCCCCn4cc(CNc5cccc6c5C(=O)N(C5CCC(=O)NC5=O)C6=O)nn4)CC3)cc2)C1]

To a solution of 2-[6-amino-5-[[(3S)-1-(4-piperazin-1-ylphenyl)-3-piperidyl]oxy]pyridazin-3-yl]phenol (40 mg, 71 μmol, TFA salt) and Ligase 28 (35 mg, 71 μmol) in THF (2 mL) was added trimethyl(phenyl)silane (8 mg, 71 μmol) followed by dibutyltin dichloride (26 mg, 85.78 μmol) and the reaction mixture was heated to 80° C. for 16 h. The reaction mixture was cooled to room temperature, diluted with water and extracted with EtOAc. The organic layer was washed with water, brine solution, dried over anhydrous sodium sulfate, filtered and concentrated under reduced pressure. The crude residue was purified by prep-HPLC to afford the title compound (3.4 mg, 3.5 μmol, 5% yield) as a pale yellow solid. MS (ESI): 925.4 ([M+H]⁺).

### Example 67

### 4-[[7-[4-[[4-[1-[3-amino-6-(2-hydroxyphenyl)pyridazin-4-yl]azetidin-3-yl]oxyphenyl]methyl]piperazin-1-yl]-7-oxo-heptyl]amino]-2-(2,6-dioxo-3-piperidyl)isoindoline-1,3-dione

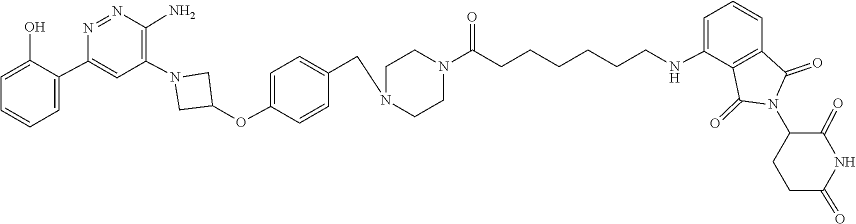 [SMILES: Nc1nnc(-c2ccccc2O)cc1N1CC(Oc2ccc(CN3CCN(C(=O)CCCCCCNc4cccc5c4C(=O)N(C4CCC(=O)NC4=O)C5=O)CC3)cc2)C1]

### a) tert-butyl 3-(4-formylphenoxy)azetidine-1-carboxylate

A mixture of 1-Boc-3-iodoazetidine (15 g, 53.2 mmol, 1.3 eq), 4-hydroxybenzaldehyde (5 g, 40.94 mmol, 1.0 eq), cesium carbonate (20 g, 61.4 mmol, 1.5 eq) in DMF (50 mL) was stirred at 80° C. for 1 h. The reaction mixture was poured into water, extracted with EtOAc, dried over Na₂SO₄ and concentrated in vacuum to afford the title compound (10 g, 88% yield) as a yellow solid.

### b) benzyl 4-(4-((1-(tert-butoxycarbonyl)azetidin-3-yl)oxy)benzyl)piperazine-1-carboxylate

A mixture of 1-Cbz-piperazine (6.35 g, 28.85 mmol, 2.0 eq), tert-butyl 3-(4-formylphenoxy)azetidine-1-carboxylate (4 g, 14.4 mmol, 1.0 eq) and acetic acid (0.5 mL, 14.4 mmol, 1.0 eq) in DME (100 mL) was stirred at 25° C. for 1 h. Then NaBH₃CN (1.8 g, 28.8 mmol, 2.0 eq) was added. The reaction mixture was stirred at 25° C. for 11 h. The reaction mixture was concentrated in vacuum and the crude product was purified by silica gel column to afford the title compound (5 g, 10.3 mmol, 72% yield) as a yellow oil. MS (ESI): 482.3 ([M+H]⁺).

### c) benzyl 4-(4-(azetidin-3-yloxy)benz)yl)piperazine-1-carboxylate

A mixture of benzyl 4-[[4-(1-tert-butoxycarbonylazetidin-3-yl)oxyphenyl]methyl]piperazine-1-carboxylate (5 g, 10.38 mmol, 1.0 eq) in TFA (10 mL, 89 mmol, 8.0 eq) and DCM (60 mL) was stirred at 25° C. for 2 h. The reaction mixture was concentrated in vacuum to afford the title compound (3.5 g, 9.18 mmol, 88% yield) as a yellow oil, trifluoroacetic acid salt. MS (ESI): 382.2 ([M+H]⁺).

### d) benzyl 4-(4-((1-(3-amino-6-chloropyridazin-4-yl)azetidin-3-yl)oxy)benzyl)piperazine-1-carboxylate

A mixture of benzyl 4-[[4-(azetidin-3-yloxy)phenyl]methyl]piperazine-1-carboxylate (3 g, 7.86 mmol, 1.0 eq), 4-bromo-6-chloro-pyridazin-3-amine (1.64 g, 7.86 mmol, 1.0 eq) and Et₃N (3.18 g, 31.46 mmol, 4.0 eq) in DMF (50 mL) was stirred at 100° C. for 2 h. The reaction mixture was concentrated in vacuum and purified by prep-HPLC to afford the title compound (2 g, 3.93 mmol, 49% yield) as a yellow oil. MS (ESI): 509.2 ([M+H]⁺).

### f) benzyl 4-(4-((1-(3-amino-6-(2-hydroxyphenyl)pyridazin-4-yl)azetidin-3-yl)oxy)benzyl) piperazine-1-carboxylate

A mixture of Ruphos-Pd-G3 (50 mg, 0.030 mmol, 0.015 eq), K₂CO₃ (815 mg, 5.89 mmol, 3.0 eq), benzyl 4-[[4-[1-(3-amino-6-chloro-pyridazin-4-yl)azetidin-3-yl]oxyphenyl]methyl] piperazine-1-carboxylate (1 g, 1.96 mmol, 1.0 eq), 2-hydroxyphenylboronic acid (542 mg, 3.93 mmol, 2.0 eq) in 1,4-dioxane (9 mL) and water (1 mL) was stirred at 90° C. for 2 h under microwave condition. The reaction mixture was concentrated in vacuum and purified by prep-HPLC to afford the title compound (1 g, 1.76 mmol, 89% yield) as a yellow oil. MS (ESI): 567.3 ([M+H]⁺).

### g) 2-(6-amino-5-(3-(4-(piperazin-1-ylmethyl)phenoxy)azetidin-1-yl)pyridazin-3-yl)phenol

A mixture of benzyl 4-[[4-[1-[3-amino-6-(2-hydroxyphenyl)pyridazin-4-yl]azetidin-3-yl]oxyphenyl]methyl]piperazine-1-carboxylate (500 mg, 0.88 mmol, 1.0 eq), 10% Pd/C (100 mg) in methanol (20 mL) was stirred at 25° C. for 24 h under H₂ atmosphere (15 psi). The reaction mixture was filtered and the filtrate was purified by prep-HPLC to afford the title compound (100 mg, 0.23 mmol, 25% yield) as a grey solid. MS (ESI): 433.8 ([M+H]⁺).

### h) 4-((7-(4-(4-((1-(3-amino-6-(2-hydroxyphenyl)pyridazin-4-yl)azetidin-3-yl)oxy)benzyl) piperazin-1-yl)-7-oxoheptyl)amino)-2-(2,6-dioxopiperidin-3-yl)isoindoline-1,3-dione

A mixture of Ligase 1 (22 mg, 0.06 mmol, 1.2 eq), Et₃N (0.5 mL, 0.14 mmol, 3.0 eq), 2-[6-amino-5-[3-[4-(piperazin-1-ylmethyl)phenoxy]azetidin-1-yl]pyridazin-3-yl]phenol (20 mg, 0.05 mmol, 1.0 eq) in DMF (2 mL) was added T₃P (25 mg, 0.06 mmol, 1.0 eq) at 25° C. The reaction mixture was stirred at 25° C. for 1 h and was purified by prep-HPLC to afford the title compound (3.2 mg, 3.92 μmol, 8% yield) as a yellow solid. MS (ESI): 816.2 ([M+H]⁺).

### Example 68

### 4-[[10-[4-[[4-[1-[3-amino-6-(2-hydroxyphenyl)pyridazin-4-yl]azetidin-3-yl]oxyphenyl]methyl]piperazin-1-yl]-10-oxo-decyl]amino]-2-(2,6-dioxo-3-piperidyl)isoindoline-1,3-dione

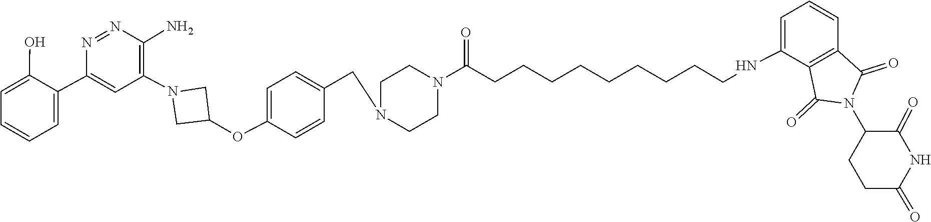 [SMILES: Nc1nnc(-c2ccccc2O)cc1N1CC(Oc2ccc(CN3CCN(C(=O)CCCCCCCCCNc4cccc5c4C(=O)N(C4CCC(=O)NC4=O)C5=O)CC3)cc2)C1]

A mixture of Ligase 23 (25 mg, 0.06 mmol, 1.2 eq), 2-[6-amino-5-[3-[4-(piperazin-1-ylmethyl)phenoxy]azetidin-1-yl]pyridazin-3-yl]phenol (20 mg, 0.05 mmol, 1.0 eq), Et₃N (0.5 mL, 0.14 mmol, 3.0 eq) in DMF (2 mL) was added T₃P (26 mg, 0.06 mmol, 1.2 eq) at 25° C. The reaction mixture was stirred at 25° C. for 1 h and was purified by prep-HPLC to afford the title compound (4.7 mg, 5.48 μmol, 11% yield) as a yellow solid. MS (ESI): 858.2 ([M+H]⁺).

### Example 69

### rac-5-[2-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]-2,6-diazaspiro[3.3]heptan-6-yl]-2-(2,6-dioxo-3-piperidyl)isoindoline-1,3-dione

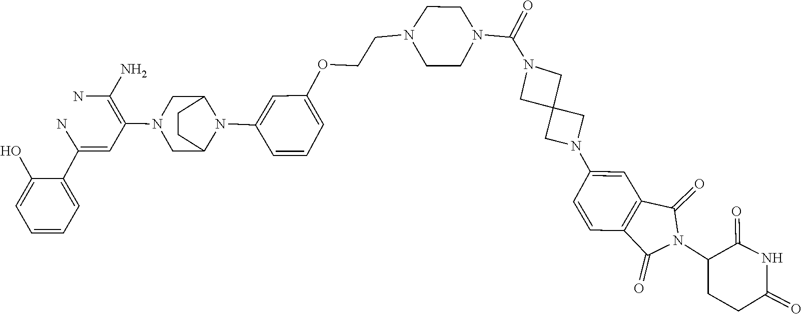

### a) benzyl 6-[2-(2,6-dioxo-3-piperidyl)-1,3-dioxo-isoindolin-5-yl]-2,6-diazaspiro[3.3]heptane-2-carboxylate

To a stirred solution of 2-(2,6-dioxo-3-piperidyl)-5-fluoro-isoindoline-1,3-dione (200 mg, 724 μmol, 1.0 eq) and benzyl 2,6-diazaspiro[3.3]heptane-2-carboxylate (168 mg, 724 μmol, 1.0 eq) in DMSO (1.5 mL) at room temperature was added DIPEA (187 mg, 253 μL, 1.45 mmol, 2.0 eq). The reaction mixture was stirred at 90° C. overnight. The reaction mixture was then poured into EtOAc/THF (1:1) and extracted sequentially with water and brine. The crude material was purified on silica column (DCM/MeOH 0-10%) to afford the title compound (83 mg, 170 μmol, 24% yield) as a yellow solid. MS (ESI): 489.3 ([M+H]⁺).

### b) 5-(2,6-diazaspiro[3.3]heptan-2-yl)-2-(2,6-dioxo-3-piperidyl)isoindoline-1,3-dione

A solution of benzyl 6-[2-(2,6-dioxo-3-piperidyl)-1,3-dioxo-isoindolin-5-yl]-2,6-diazaspiro[3.3]heptane-2-carboxylate (83 mg, 170 μmol, 1.0 eq) in methanol (20 mL) was degassed with argon for 10 min. 10% Pd on charcoal (21.7 mg, 20.4 μmol, 0.12 eq) was added. The reaction mixture was degassed with H₂ for 10 min. The reaction mixture was then stirred under a H₂ balloon for 4 h. The reaction mixture was filtered and the filtrate was concentrated in vacuo to afford the title compound (75 mg, 106 μmol, 62% yield) as a yellow solid. MS (ESI): 355.2 ([M+H]⁺).

### c) rac-5-[2-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]-2,6-diazaspiro[3.3]heptan-6-yl]-2-(2,6-dioxo-3-piperidyl)isoindoline-1,3-dione

To a stirred solution of (4-nitrophenyl) 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate (35 mg, 52.5 μmol, 1.0 eq) and Ligase 46 (37.2 mg, 105 μmol, 2.0 eq) in DMSO (0.6 mL) at room temperature was added DIPEA (17 mg, 22.9 μL, 131 μmol, 2.5 eq). The reaction mixture was stirred at 115° C. for 20 h. The reaction mixture was poured into THF/EtOAc (1:1) and washed sequentially with water and with brine. The organic layer was dried over Na₂SO₄ and concentrated in vacuo. The crude material was purified on silica column (DCM/MeOH 0-5%) to afford the title compound (3 mg, 3.4 μmol, 6% yield) as a yellow solid. MS (ESI): 882.4 ([M+H]⁺).

### Example 70

### rac-5-[4-[4-[[3-[3-[3-amino-6-(2-hydroxyphenyl)pyridazin-4-yl]-3,8-diazabicyclo[3.2.1]octan-8-yl]phenyl]methyl]piperidine-1-carbonyl]-1-piperidyl]-2-(2,6-dioxo-3-piperidyl)isoindoline-1,3-dione

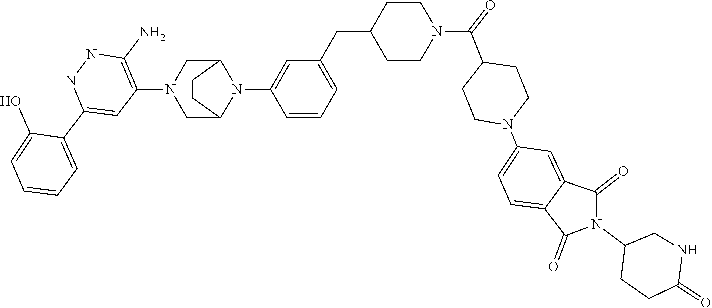 [SMILES: NC1=C(N2CC3CCC(C2)N3c2cccc(CC3CCN(C(=O)C4CCN(c5ccc6c(c5)C(=O)N(C5CCC(=O)NC5)C6=O)CC4)CC3)c2)C=C(c2ccccc2O)NN1]

To a stirred solution of 2-[6-amino-5-[8-[3-(4-piperidylmethyl)phenyl]-3,8-diazabicyclo[3.2.1]octan-3-yl]pyridazin-3-yl]phenol (14 mg, 29.7 μmol, 1.0 eq), Ligase 15 (12.6 mg, 32.7 μmol, 1.1 eq) and DIPEA (15.4 mg, 20.8 μL, 119 μmol, 4.0 eq) in DMSO (0.5 mL) at room temperature was added HATU (22.6 mg, 59.5 μmol, 2.0 eq). The reaction mixture was stirred at room temperature for 1 h. The reaction mixture was poured into THF/EtOAc (1:1) and washed sequentially with water and with brine. The organic layer was dried over Na₂SO₄ and concentrated in vacuo. The crude material was purified on silica column (DCM/MeOH 0-5%) to afford the title compound (13 mg, 15.5 μmol, 52% yield) as a yellow solid. MS (ESI): 838.4 ([M+H]⁺).

### Example 71

### N-[[1-[3-amino-6-(2-hydroxyphenyl)pyridazin-4-yl]-4-phenyl-4-piperidyl]methyl]-1-[2-(2,6-dioxo-3-piperidyl)-1,3-dioxo-isoindolin-4-yl]piperidine-4-carboxamide

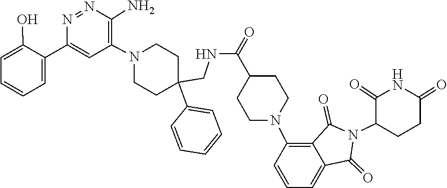 [SMILES: Nc1nnc(-c2ccccc2O)cc1N1CCC(CNC(=O)C2CCN(c3cccc4c3C(=O)N(C3CCC(=O)NC3=O)C4=O)CC2)(c2ccccc2)CC1]

### a) tert-butyl N-[[1-(3-amino-6-chloro-pyridazin-4-yl)-4-phenyl-4-piperidyl]methyl]carbamate

To a solution of 4-bromo-6-chloropyridazin-3-amine (1.0 g, 4.80 mmol, 1.0 eq) and tert-butyl ((4-phenylpiperidin-4-yl)methyl)carbamate (1.46 g, 5.04 mmol, 1.05 eq) in DMSO (15 mL) was added potassium carbonate (3.98 g, 28.8 mmol, 6.0 eq) and the reaction mixture was stirred at 100° C. for 20 h. The reaction mixture was cooled to room temperature and slowly added to water (50 ml) while stirring. The precipitate was filtered through glass fiber paper and the solid was collected and dried in vacuo to afford the title compound (1.9 g, 4.56 mmol μmol, 95% yield) as a brown solid. MS (ESI): 418.3 ([M+H]⁺).

### b) tert-butyl N-[[1-[3-amino-6-(2-hydroxyphenyl)pyridazin-4-yl]-4-phenyl-4-piperidyl]methyl]carbamate

A suspension of tert-butyl ((1-(3-amino-6-chloropyridazin-4-yl)-4-phenylpiperidin-4-yl)methyl)carbamate (285 mg, 682 μmol, 1.0 eq), (2-hydroxyphenyl)boronic acid (235 mg, 1.7 mmol, 2.5 eq), potassium carbonate (283 mg, 2.05 mmol, 3.0 eq) and RuPhos Pd G3 (17.1 mg, 20.5 μmol, 0.03 eq) in a mixture of degassed dioxane (10 mL) and water (1 mL) was stirred at 110° C. for 2 h under argon. The reaction mixture was poured in saturated NaHCO₃ and extracted with EtOAc. The organic layers were combined, washed with water and brine. The organic layers were dried over Na₂SO₄ and concentrated in vacuo. The crude material was purified on silica column (heptane/EtOAc 0-100%) to afford the title compound (230 mg, 484 μmol, 71% yield) as a brown foam. MS (ESI): 476.4 ([M+H]⁺).

### c) 2-[6-amino-5-[4-(aminomethyl)-4-phenyl-1-piperidyl]pyridazin-3-yl]phenol

To a cooled (0° C.) solution of tert-butyl ((1-(3-amino-6-(2-hydroxyphenyl)pyridazin-4-yl)-4-phenylpiperidin-4-yl)methyl)carbamate (225 mg, 473 umol, 1.0 eq) in DCM (5 mL) was added 4M HCl in dioxane (473 μL, 1.89 mmol, 4.0 eq). The reaction mixture was allowed to reach room temperature and stirred for 20 h. The reaction mixture was filtered and the solid was dried in vacuo to afford the title compound (175 mg, 424 μmol, 90% yield) as a white solid, hydrochloride salt. MS (ESI): 376.3 ([M+H]⁺).

### d) N-[[1-[3-amino-6-(2-hydroxyphenyl)pyridazin-4-yl]-4-phenyl-4-piperidyl]methyl]-1-[2-(2,6-dioxo-3-piperidyl)-1,3-dioxo-isoindolin-4-yl]piperidine-4-carboxamide

Ligase 11 (15.4 mg, 40.1 μmol, 1.1 eq) was dissolved in dry DMF (200 μL). DIPEA (11.8 mg, 15.9 μL, 91 μmol, 2.5 eq) and HATU (16.6 mg, 43.7 μmol, 1.2 eq) were added and the mixture was stirred at room temperature for 10 min. 2-(6-amino-5-(4-(aminomethyl)-4-phenylpiperidin-1-yl)pyridazin-3-yl)phenol, hydrochloride salt (15 mg, 36.4 μmol, 1.0 eq) was added and the mixture was stirred at room temperature for 2 h. The reaction mixture was purified directly by prep-HPLC to afford the title compound (24 mg, 28 μmol, 76% yield) as a yellow solid, 2,2,2-trifluoroacetate salt. MS (ESI): 743.4 ([M+H]⁺).

### Example 72

### N-[[1-[3-amino-6-(2-hydroxyphenyl)pyridazin-4-yl]-4-phenyl-4-piperidyl]methyl]-2-[2-(2,6-dioxo-3-piperidyl)-1,3-dioxo-isoindolin-4-yl]oxy-acetamide

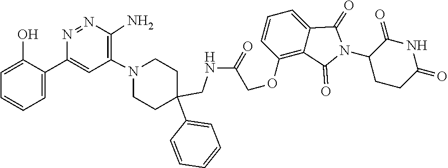 [SMILES: Nc1nnc(-c2ccccc2O)cc1N1CCC(CNC(=O)COc2cccc3c2C(=O)N(C2CCC(=O)NC2=O)C3=O)(c2ccccc2)CC1]

The title compound was prepared in analogy to example 71 step d using Ligase 47 as a light brown solid (11 mg, 13.7 μmol, 37% yield), 2,2,2-trifluoroacetate salt. MS (ESI): 690.6 ([M+H]⁺).

### Example 73

### N-((1-(3-amino-6-(2-hydroxyphenyl)pyridazin-4-yl)-4-phenylpiperidin-4-yl)methyl)-2-((2-(2,6-dioxopiperidin-3-yl)-1,3-dioxoisoindolin-5-yl)oxy)acetamide

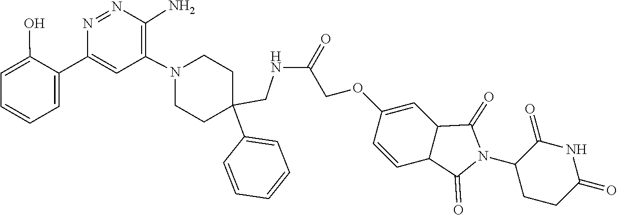 [SMILES: Nc1nnc(-c2ccccc2O)cc1N1CCC(CNC(=O)COC2=CC3C(=O)N(C4CCC(=O)NC4=O)C(=O)C3C=C2)(c2ccccc2)CC1]

The title compound was prepared in analogy to example 71 step d using Ligase 48 as a light yellow solid (11 mg, 13.7 μmol, 37% yield), 2,2,2-trifluoroacetate salt. MS (ESI): 690.5 ([M+H]⁺).

### Example 74

### N-((1-(3-amino-6-(2-hydroxyphenyl)pyridazin-4-yl)-4-phenylpiperidin-4-yl)methyl)-1-(2-(2,6-dioxopiperidin-3-yl)-1,3-dioxoisoindolin-5-yl)piperidine-4-carboxamide

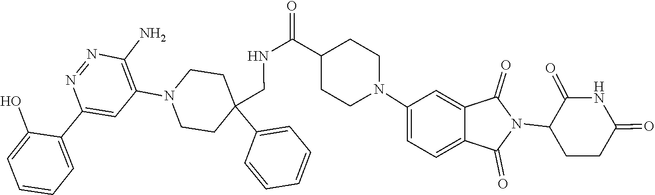 [SMILES: Nc1nnc(-c2ccccc2O)cc1N1CCC(CNC(=O)C2CCN(c3ccc4c(c3)C(=O)N(C3CCC(=O)NC3=O)C4=O)CC2)(c2ccccc2)CC1]

The title compound was prepared in analogy to example 71 step d using Ligase 15 as a light brown solid 15 (12 mg, 13.3 μmol, 36% yield), 2,2,2-trifluoroacetate salt. MS (ESI): 743.5 ([M+H]⁺).

### Example 75

### N-((1-(3-amino-6-(2-hydroxyphenyl)pyridazin-4-yl)-4-phenylpiperidin-4-yl)methyl)-3-((2-(2,6-dioxopiperidin-3-yl)-1,3-dioxoisoindolin-4-yl)amino)

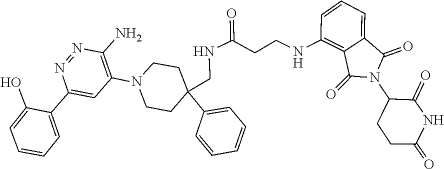 [SMILES: Nc1nnc(-c2ccccc2O)cc1N1CCC(CNC(=O)CCNc2cccc3c2C(=O)N(C2CCC(=O)NC2=O)C3=O)(c2ccccc2)CC1]

The title compound was prepared in analogy to example 71 step d using Ligase 43 as a light brown solid (14 mg, 16.3 μmol, 44% yield), 2,2,2-trifluoroacetate salt. MS (ESI): 703.4 ([M+H]⁺).

### Example 76

### N-((1-(3-amino-6-(2-hydroxyphenyl)pyridazin-4-yl)-4-phenylpiperidin-4-yl)methyl)-2-((2-(2,6-dioxopiperidin-3-yl)-1,3-dioxoisoindolin-4-yl)amino)acetamide

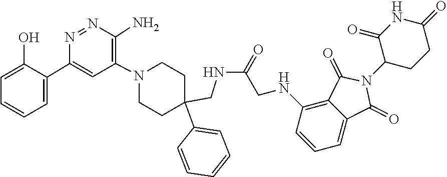 [SMILES: Nc1nnc(-c2ccccc2O)cc1N1CCC(CNC(=O)CNc2cccc3c2C(=O)N(C2CCC(=O)NC2=O)C3=O)(c2ccccc2)CC1]

The title compound was prepared in analogy to example 71 step d using Ligase 49 as a light brown solid (15 mg, 17.8 μmol, 48% yield), 2,2,2-trifluoroacetate salt. MS (ESI): 689.5 ([M+H]⁺).

### Example 77

### N-((1-(3-amino-6-(2-hydroxyphenyl)pyridazin-4-yl)-4-phenylpiperidin-4-yl)methyl)-2-(1-(2-(2,6-dioxopiperidin-3-yl)-1,3-dioxoisoindolin-5-yl)piperidin-4-yl)acetamide

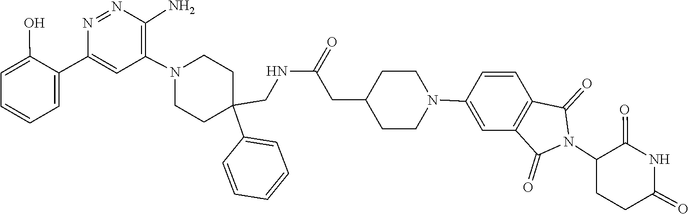 [SMILES: Nc1nnc(-c2ccccc2O)cc1N1CCC(CNC(=O)CC2CCN(c3ccc4c(c3)C(=O)N(C3CCC(=O)NC3=O)C4=O)CC2)(c2ccccc2)CC1]

The title compound was prepared in analogy to example 71 step d using Ligase 24 as a light brown solid (15 mg, 16.4 μmol, 44% yield), 2,2,2-trifluoroacetate salt. MS (ESI): 757.6 ([M+H]⁺).

### Example 78

### N-((1-(3-amino-6-(2-hydroxyphenyl)pyridazin-4-yl)-4-phenylpiperidin-4-yl)methyl)-4-(4-(4-((2,6-dioxopiperidin-3-yl)oxy)phenyl)piperidin-1-yl)butanamide

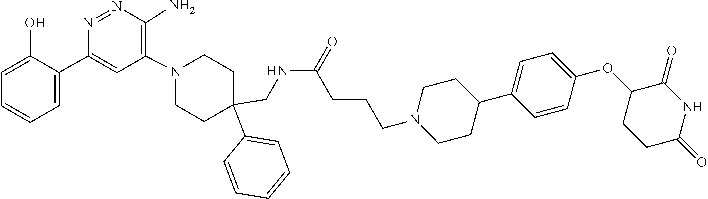 [SMILES: Nc1nnc(-c2ccccc2O)cc1N1CCC(CNC(=O)CCCN2CCC(c3ccc(OC4CCC(=O)NC4=O)cc3)CC2)(c2ccccc2)CC1]

The title compound was prepared in analogy to example 71 step d using Ligase 39 as a light brown solid (14 mg, 15.7 μmol, 43% yield), 2,2,2-trifluoroacetate salt. MS (ESI): 732.6 ([M+H]⁺).

### Example 79

### rac-3-[4-[1-[2-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-2-oxo-ethyl]-4-piperidyl]anilino]piperidine-2,6-dione

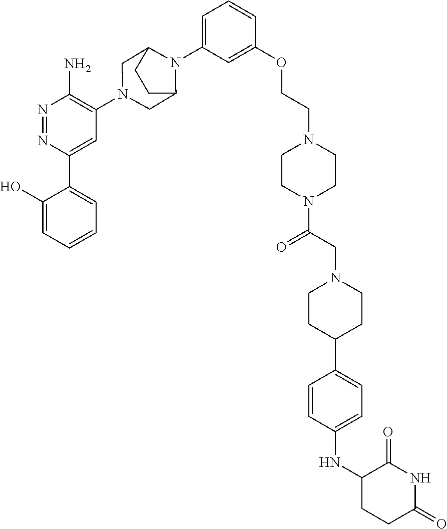 [SMILES: Nc1nnc(-c2ccccc2O)cc1N1CC2CCC(C1)N2c1cccc(OCCN2CCN(C(=O)CN3CCC(c4ccc(NC5CCC(=O)NC5=O)cc4)CC3)CC2)c1]

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (50 mg, 99.7 μmol, 1.0 eq), Ligase 51 (38.1 mg, 99.7 umol, 1.0 eq) and HATU (79.6 mg, 209 μmol, 2.1 eq) in DMSO (1 mL) at room temperature was added DIPEA (38.6 mg, 52.2 μL, 299 μmol, 3.0 eq). The reaction mixture was stirred for 3 h. The reaction mixture was partitioned between EtOAc/THF (1:1) and water. The layers were separated. The aqueous layer was extracted with EtOAc/THF. The combined organic layers were washed with brine, dried over anhydrous sodium sulfate, and concentrated in vacuo. The crude material was purified on silica column (DCM/MeOH 0-10%) to afford the title compound (38.7 mg, 46.7 μmol, 47% yield) as an off-white solid. MS (ESI): 828.1 ([M−H]⁻).

### Example 80

### rac-3-[4-[1-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]-4-piperidyl]anilino]piperidine-2,6-dione

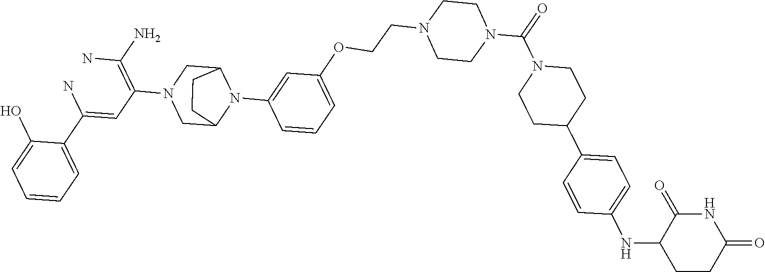 [SMILES: NC(N)=C(/C=C(\N)c1ccccc1O)N1CC2CCC(C1)N2c1cccc(OCCN2CCN(C(=O)N3CCC(c4ccc(NC5CCC(=O)NC5=O)cc4)CC3)CC2)c1]

To a stirred solution of (4-nitrophenyl) 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate (35 mg, 52.5 μmol, 1.0 eq) and Ligase 50 (18.7 mg, 57.7 μmol, 1.1 eq) in DMSO (0.6 mL) at room temperature was added DIPEA (17 mg, 22.9 μL, 131 μmol, 2.5 eq). The reaction mixture was stirred at 115° C. for 24 h. The crude material was purified by prep-HPLC, followed on silica column (DCM/MeOH 0-5%) to afford the title compound (11 mg, 10.5 μmol, 20% yield) as an off-white solid, bis-(2,2,2-trifluoroacetic acid)salt. MS (ESI): 815.4 ([M+H]⁺).

### Example 81

### rac-3-[4-[4-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]piperazin-1-yl]anilino]piperidine-2,6-dione

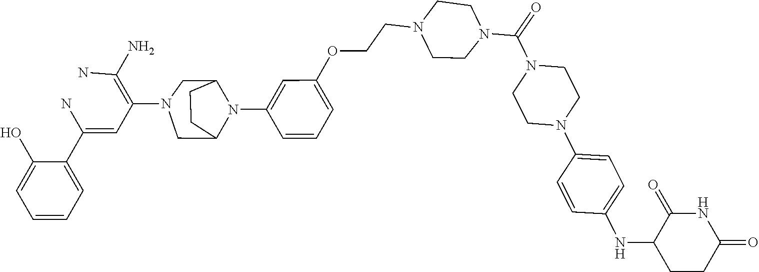 [SMILES: NC(N)=C(/C=C(\N)c1ccccc1O)N1CC2CCC(C1)N2c1cccc(OCCN2CCN(C(=O)N3CCN(c4ccc(NC5CCC(=O)NC5=O)cc4)CC3)CC2)c1]

To a stirred solution of (4-nitrophenyl) 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate (35 mg, 52.5 μmol, 1.0 eq) and Ligase 53 (18.8 mg, 57.7 μmol, 1.1 eq) in DMSO (0.6 mL) at room temperature was added DIPEA (17 mg, 22.9 μL, 131 μmol, 2.5 eq). The reaction mixture was stirred at 115° C. for 24 h. The crude material was purified by prep-HPLC, followed on silica column (DCM/MeOH 0-5%) to afford the title compound (7 mg, 6.7 μmol, 13% yield)) as an off-white solid, bis-(2,2,2-trifluoroacetic acid)salt. MS (ESI): 816.4 ([M+H]⁺).

### Example 82

### 4-(3-amino-6-(2-hydroxyphenyl)pyridazin-4-yl)-N-((1-(1-(2-(2,6-dioxopiperidin-3-yl)-1,3-dioxoisoindolin-5-yl)piperidine-4-carbonyl)piperidin-4-yl)methyl)-1-phenylpiperazine-2-carboxamide

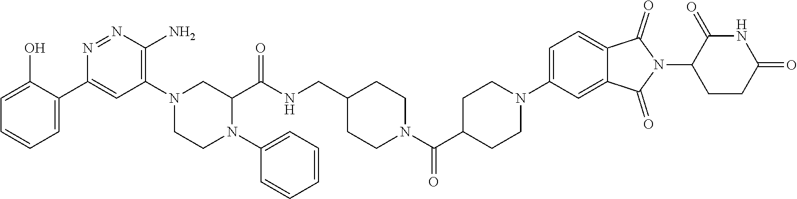 [SMILES: Nc1nnc(-c2ccccc2O)cc1N1CCN(c2ccccc2)C(C(=O)NCC2CCN(C(=O)C3CCN(c4ccc5c(c4)C(=O)N(C4CCC(=O)NC4=O)C5=O)CC3)CC2)C1]

### a) potassium 4-(3-amino-6-chloro-pyridazin-4-yl)-1-phenyl-piperazine-2-carboxylate

To a mixture of 1-phenylpiperazine-2-carboxylic acid dihydrochloride (590 mg, 2.12 mmol, 1.1 eq) and 4-bromo-6-chloropyridazin-3-amine (400 mg, 1.92 mmol, 1.0 eq) in DMA (6 mL) was added K₂CO₃ (530 mg, 3.84 mmol, 2.0 eq). The reaction mixture was stirred at 120° C. for 20 h. The reaction mixture was concentrated to afford the title compound (600 mg, 1.80 mmol. 94% yield) as a brown oil. MS (ESI): 334.2 ([M+H]⁺).

### b) tert-butyl 4-[[[4-(3-amino-6-chloro-pyridazin-4-yl)-1-phenyl-piperazine-2-carbonyl]amino]methyl]piperidine-1-carboxylate

To a solution of potassium 4-(3-amino-6-chloro-pyridazin-4-yl)-1-phenyl-piperazine-2-carboxylate (400 mg, 1.2 mmol, 1.0 eq), HATU (684 mg, 1.8 mmol, 1.5 eq) and DIPEA (774 mg, 1.05 mL, 5.99 mmol, 5.0 eq) in DMF (5 mL) was added tert-butyl 4-(aminomethyl)piperidine-1-carboxylate (385 mg, 1.8 mmol, 1.5 eq). The reaction mixture was stirred at room temperature for 5 h. The reaction mixture was concentrated in vacuo. The crude material was purified on silica column (heptane/EtOAc 0-100%) to afford the title compound (500 mg, 945 μmol, 79% yield) as a brown solid. MS (ESI): 530.0 ([M+H]⁺).

### c) tert-butyl 4-[[[4-[3-amino-6-(2-hydroxyphenyl)pyridazin-4-yl]-1-phenyl-piperazine-2-carbonyl]amino]methyl]piperidine-1-carboxylate

A suspension of tert-butyl 4-((4-(3-amino-6-chloropyridazin-4-yl)-1-phenylpiperazine-2-carboxamido)methyl)piperidine-1-carboxylate (60 mg, 113 μmol, 1.0 eq), (2-hydroxyphenyl)boronic acid (31.2 mg, 226 μmol, 2.0 eq), K₂CO₃ (46.9 mg, 340 μmol, 3.0 eq) and RuPhos Pd G3 (4.73 mg, 5.66 μmol, 0.05 eq) in a mixture of degassed dioxane (3 mL) and water (0.3 mL) was stirred at 110° C. for 2 h under argon. The reaction mixture was poured in saturated NaHCO₃ and extracted with EtOAc. The organic layers were combined, washed with water and brine. The organic layers were dried over Na₂SO₄ and concentrated in vacuo. The crude material was purified on silica column (heptane/EtOAc 0-100%) to afford the title compound (35 mg, 60 μmol, 53% yield) as a brown foam. MS (ESI): 588.5 ([M+H]⁺).

### d) 4-[3-amino-6-(2-hydroxyphenyl)pyridazin-4-yl]-1-phenyl-N-(4-piperidylmethyl)piperazine-2-carboxamide

To a cooled (0° C.) solution of tert-butyl 4-((4-(3-amino-6-(2-hydroxyphenyl)pyridazin-4-yl)-1-phenylpiperazine-2-carboxamido)methyl)piperidine-1-carboxylate (80 mg, 136 μmol, 1.0 eq) in DCM (3 mL) was added 4M HCl in dioxane (102 μL, 408 μmol, 3.0 eq). The reaction mixture was allowed to reach room temperature and stirred for 20 h. The reaction mixture was filtered and the solid was dried in vacuo to afford the title compound (70 mg, 133 μmol, 100% yield) as a white solid, hydrochloride salt. MS (ESI): 488.4 ([M+H]⁺).

### e) 4-(3-amino-6-(2-hydroxyphenyl)pyridazin-4-yl)-N-((1-(1-(2-(2,6-dioxopiperidin-3-yl)-1,3-dioxoisoindolin-5-yl)piperidine-4-carbonyl)piperidin-4-yl)methyl)-1-phenylpiperazine-2-carboxamide

Ligase 15 (10 mg, 25.9 μmol, 1.0 eq) was dissolved in dry DMF (200 μL). DIPEA (8.38 mg, 11.3 μL, 64.9 μmol, 2.5 eq) and HATU (11.8 mg, 31.1 μmol, 1.2 eq) were added and the mixture was stirred at room temperature for 10 min. 4-(3-amino-6-(2-hydroxyphenyl)pyridazin-4-yl)-1-phenyl-N-(piperidin-4-ylmethyl)piperazine-2-carboxamide, hydrochloride salt (13.6 mg, 25.9 μmol, 1.0 eq) was added and the mixture was stirred at room temperature for 2 h. The reaction mixture was purified directly by prep-HPLC to afford the title compound (7.5 mg, 7.74 μmol, 29% yield) as a light yellow solid, 2,2,2-trifluoroacetate salt. MS (ESI): 855.7 ([M+H]⁺).

### Example 83

### 5-(4-(4-(2-(3-(9-(3-amino-6-(2-hydroxyphenyl)pyridazin-4-yl)-1-oxa-4,9-diazaspiro[5.5]undecan-4-yl)phenoxy)ethyl)piperazine-1-carbonyl)piperidin-1-yl)-2-(2,6-dioxopiperidin-3-yl)isoindoline-1,3-dione

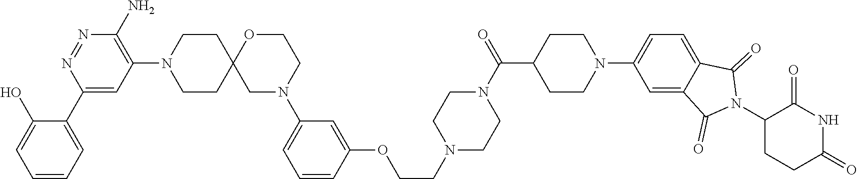 [SMILES: Nc1nnc(-c2ccccc2O)cc1N1CCC2(CC1)CN(c1cccc(OCCN3CCN(C(=O)C4CCN(c5ccc6c(c5)C(=O)N(C5CCC(=O)NC5=O)C6=O)CC4)CC3)c1)CCO2]

### a) tert-butyl 4-[2-(3-bromophenoxy)ethyl]piperazine-1-carboxylate

To a solution of 3-bromophenol (1.5 g, 8.67 mmol, 1.0 eq), tert-butyl 4-(2-hydroxyethyl)piperazine-1-carboxylate (2.2 g, 9.54 mmol, 1.1 eq) and triphenylphosphine (2.5 g, 9.54 mmol, 1.1 eq) in THF (25 mL) was added di-tert-butyl azodicarboxylate (2.2 g, 9.54 mmol, 1.1 eq). The reaction mixture was stirred for 2 h at room temperature. The reaction mixture was poured in EtOAc and washed with water and brine. The organic layer was dried over Na₂SO₄ and concentrated. The crude material was purified on silica column (heptane/EtOAc 0-50%) to afford the title compound (2.93 g, 7.6 mmol, 88% yield) as a colorless oil. MS (ESI): 387.1 ([M+H]⁺).

### b) benzyl 4-[3-[2-(4-tert-butoxycarbonylpiperazin-1-yl)ethoxy]phenyl]-1-oxa-4,9-diazaspiro[5.5]undecane-9-carboxylate

Palladium (II) acetate (33 mg, 147 μmol, 0.2 eq) and Ruphos (34.3 mg, 73.4 μmol, 0.10 eq) were combined in degassed toluene (4 mL) under argon. The reaction mixture was heated to 50° C. and stirred for 20 min. In a separate flask flushed with argon, tert-butyl 4-(2-(3-bromophenoxy)ethyl)piperazine-1-carboxylate (283 mg, 734 μmol, Eq: 1.00), benzyl 1-oxa-4,9-diazaspiro[5.5]undecane-9-carboxylate hydrochloride (300 mg, 734 μmol, Eq: 1) and sodium tert-butoxide (212 mg, 2.2 mmol, 3.0 eq) were combined in degassed toluene (4 mL) under argon. The reaction mixture was heated to 50° C. and the catalyst reaction mixture was added via a seringe. The reaction mixture was stirred at 100° C. for 16 h. The reaction mixture was poured into saturated NaHCO₃ and extracted with EtOAc. The organic layers were combined and washed with water and brine. The organic layers were dried over Na₂SO₄ and concentrated in vacuo. The crude material was purified on silica column (heptane/EtOAc 0-100%) to afford the title compound (330 mg, 555 μmol, 68% yield) as a light brown oil. MS (ESI): 595.4 ([M+H]⁺).

### c) tert-butyl 4-[2-[3-(1-oxa-4,9-diazaspiro[5.5]undecan-4-yl)phenoxy]ethyl]piperazine-1-carboxylate

To a solution of benzyl 4-(3-(2-(4-(tert-butoxycarbonyl)piperazin-1-yl)ethoxy)phenyl)-1-oxa-4,9-diazaspiro[5.5]undecane-9-carboxylate (500 mg, 841 μmol, 1.0 eq) in methanol (5 mL) was added 10% palladium on charcoal (89.5 mg, 84.1 μmol, 0.1 eq). The reaction mixture was vigorously stirred at room temperature for 3 h under H₂ (baloon). The catalyst was collected by filtration, washing with methanol. The filtrate was concentrated to afford the title compound (390 mg, 840 μmol, 100% yield) as a light brown oil. MS (ESI): 461.4 ([M+H]⁺).

### d) tert-butyl 4-[2-[3-[9-(3-amino-6-chloro-pyridazin-4-yl)-1-oxa-4,9-diazaspiro[5.5]undecan-4-yl]phenoxy]ethyl]piperazine-1-carboxylate

To a stirred solution of 4-bromo-6-chloropyridazin-3-amine (180 mg, 864 μmol, 1.0 eq) and tert-butyl 4-(2-(3-(1-oxa-4,9-diazaspiro[5.5]undecan-4-yl)phenoxy)ethyl)piperazine-1-carboxylate (398 mg, 864 μmol, 1.0 eq) in DMA (6 mL) was added potassium carbonate (239 mg, 1.73 mmol, 2.0 eq). The reaction mixture was heated to 110° C. and stirred for 16 h. The reaction mixture was poured in water and extracted with EtOAc. The organic layers were combined, washed with saturated NaHCO₃, water and brine. The organic layers were dried over Na₂SO₄ and concentrated in vacuo. The crude material was purified on silica column (DCM/MeOH 0-10%) to afford the title compound (340 mg, 578 μmol, 57% yield) as a brown oil. MS (ESI): 588.4 ([M+H]⁺).

### e) tert-butyl 4-[2-[3-[9-[3-amino-6-(2-hydroxyphenyl)pyridazin-4-yl]-1-oxa-4,9-diazaspiro[5.5]undecan-4-yl]phenoxy]ethyl]piperazine-1-carboxylate

A suspension of tert-butyl 4-(2-(3-(9-(3-amino-6-chloropyridazin-4-yl)-1-oxa-4,9-diazaspiro[5.5]undecan-4-yl)phenoxy)ethyl)piperazine-1-carboxylate (340 mg, 578 μmol, 1.0 eq), (2-hydroxyphenyl)boronic acid (199 mg, 1.45 mmol, 2.5 eq) and potassium carbonate (240 mg, 1.73 mmol, 3.0 eq) and RuPhos Pd G3 (24.2 mg, 28.9 μmol, 0.05 eq) in a mixture of degassed dioxane (10 mL) and water (1 mL) was stirred at 120° C. for 16 h. The reaction mixture was poured in saturated NaHCO₃ and extracted with EtOAc. The organic layers were combined, washed with water and brine. The organic layers were dried over Na₂SO₄ and concentrated in vacuo. The crude material was purified on silica column (DCM/MeOH 0-5%) to afford the title compound (120 mg, 186 μmol, 29% yield) as a yellow solid. MS (ESI): 646.5 ([M+H]⁺).

### f) 2-[6-amino-5-[4-[3-(2-piperazin-1-ylethoxy)phenyl]-1-oxa-4,9-diazaspiro[5.5]undecan-9-yl]pyridazin-3-yl]phenol

To a cooled (0° C.) solution of tert-butyl 4-(2-(3-(9-(3-amino-6-(2-hydroxyphenyl)pyridazin-4-yl)-1-oxa-4,9-diazaspiro[5.5]undecan-4-yl)phenoxy)ethyl)piperazine-1-carboxylate (120 mg, 186 μmol, 1.0 eq) in DCM (3 mL) was added 4M HCl in dioxane (139 μL, 557 μmol, 3.0 eq). The reaction mixture was allowed to reach room temperature and stirred for 20 h. The reaction mixture was concentrated in vacuo to afford the title compound (100 mg, 183 μmol, 95% yield) as a white solid, hydrochloride salt. MS (ESI): 546.4 ([M+H]⁺).

### g) 5-(4-(4-(2-(3-(9-(3-amino-6-(2-hydroxyphenyl)pyridazin-4-yl)-1-oxa-4,9-diazaspiro[5.5]undecan-4-yl)phenoxy)ethyl)piperazine-1-carbonyl)piperidin-1-yl)-2-(2,6-dioxopiperidin-3-yl)isoindoline-1,3-dione

Ligase 15 (10 mg, 25.9 μmol, 1.0 eq) was dissolved in dry DMF (200 μL). DIPEA (8.38 mg, 11.3 μL, 64.9 μmol, 2.5 eq) and HATU (11.8 mg, 31.1 μmol, 1.2 eq) were added and the mixture was stirred at room temperature for 10 min. 2-(6-amino-5-(4-(3-(2-(piperazin-1-yl)ethoxy)phenyl)-1-oxa-4,9-diazaspiro[5.5]undecan-9-yl)pyridazin-3-yl)phenol hydrochloride (15.1 mg, 25.9 μmol, 1.0 eq) was added and the mixture was stirred at room temperature for 2 h. The reaction mixture was purified directly by prep-HPLC (basic) to afford the title compound (6.7 mg, 7.34 μmol, 28% yield) as a light yellow solid. MS (ESI): 913.7 ([M+H]⁺).

### Example 84

### 5-(4-(4-(2-(3-(7-(3-amino-6-(2-hydroxyphenyl)pyridazin-4-yl)-4,7-diazaspiro[2.5]octan-4-yl)phenoxy)ethyl)piperazine-1-carbonyl)piperidin-1-yl)-2-(2,6-dioxopiperidin-3-yl)isoindoline-1,3-dione

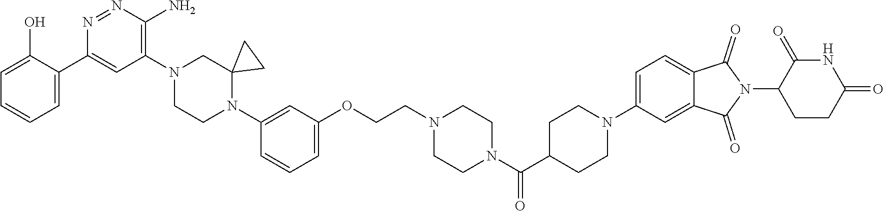 [SMILES: Nc1nnc(-c2ccccc2O)cc1N1CCN(c2cccc(OCCN3CCN(C(=O)C4CCN(c5ccc6c(c5)C(=O)N(C5CCC(=O)NC5=O)C6=O)CC4)CC3)c2)C2(CC2)C1]

The title compound was prepared in analogy to example 5 step f using Ligase 15 as a light yellow solid (5.9 mg, 6.79 μmol, 26% yield). MS (ESI): 869.6 ([M+H]⁺).

### Example 85

### 4-[3-amino-6-(2-hydroxyphenyl)pyridazin-4-yl]-N-[[4-[[1-[2-(2,6-dioxopiperidin-3-yl)-1,3-dioxoisoindol-5-yl]piperidine-4-carbonyl]amino]cyclohexyl]methyl]-1-phenylpiperazine-2-carboxamide

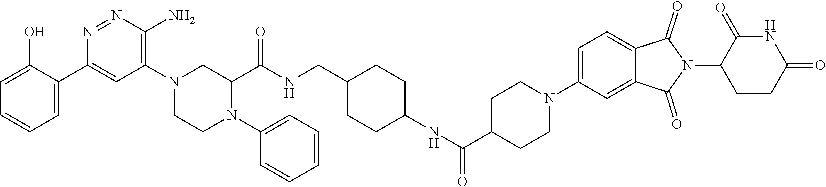 [SMILES: Nc1nnc(-c2ccccc2O)cc1N1CCN(c2ccccc2)C(C(=O)NCC2CCC(NC(=O)C3CCN(c4ccc5c(c4)C(=O)N(C4CCC(=O)NC4=O)C5=O)CC3)CC2)C1]

### a) benzyl 3-[[4-(tert-butoxycarbonylamino)cyclohexyl]methylcarbamoyl]-4-phenyl-piperazine-1-carboxylate

To a solution of 4-((benzyloxy)carbonyl)-1-phenylpiperazine-2-carboxylic acid (700 mg, 2.06 mmol, 1.0 eq), HATU (977 mg, 2.57 mmol, 1.25 eq) and DIPEA (1.33 g, 1.80 mL, 10.3 mmol, 5.0 eq) in DMF (8 mL) was added rac-tert-butyl (-4-(aminomethyl)cyclohexyl)carbamate (517 mg, 2.26 mmol, 1.1 eq). The reaction mixture was stirred at room temperature for 2 h. The reaction mixture was concentrated in vacuo. The crude material was purified on silica column (heptane/EtOAc 0-100%) to afford the title compound (435 mg, 790 μmol, 38% yield) as a white foam. MS (ESI): 551.4 ([M+H]⁺).

### b) tert-butyl N-[4-[[(1-phenylpiperazine-2-carbonyl)amino]methyl]cyclohexyl]carbamate

To a solution of benzyl 3-(((-4-((tert-butoxycarbonyl)amino)cyclohexyl)methyl)carbamoyl)-4-phenylpiperazine-1-carboxylate (435 mg, 790 μmol, 1.0 eq) in methanol (10 mL) was added 10% palladium on charcoal (84.1 mg, 79 μmol, 0.1 eq). The reaction mixture was vigorously stirred at room temperature for 24 h under H₂ (baloon). The catalyst was collected by filtration and washed with methanol. The filtrate was concentrated to afford the title compound (320 mg, 769 μmol, 97% yield) as a grey foam. MS (ESI): 417.4 ([M+H]⁺).

### c) tert-butyl N-[4-[[[4-(3-amino-6-chloro-pyridazin-4-yl)-1-phenyl-piperazine-2-carbonyl]amino]methyl]cyclohexyl]carbamate

To a stirred solution of 4-bromo-6-chloropyridazin-3-amine (173 mg, 832 μmol, 1.0 eq) and rac-tert-butyl ((1r,4r)-4-((1-phenylpiperazine-2-carboxamido)methyl)cyclohexyl)carbamate (315 mg, 756 μmol, 1.0 eq) in DMA (4 mL) was added potassium carbonate (523 mg, 3.78 mmol, 5.0 eq). The reaction mixture was heated to 110° C. and stirred for 20 h. The reaction mixture was poured in water and extracted with EtOAc. The organic layers were combined, washed with saturated NaHCO₃, water and brine. The organic layers were dried over Na₂SO₄ and concentrated in vacuo. The crude material was purified on silica column (heptane/EtOAc 0-100%) to afford the title compound (85 mg, 156 μmol, 19% yield) as a light brown solid. MS (ESI): 544.4 ([M+H]⁺).

### d) tert-butyl N-[4-[[[4-[3-amino-6-(2-hydroxyphenyl)pyridazin-4-yl]-1-phenyl-piperazine-2-carbonyl]amino]methyl]cyclohexyl]carbamate

A suspension of tert-butyl N-[4-[[[4-(3-amino-6-chloro-pyridazin-4-yl)-1-phenyl-piperazine-2-carbonyl]amino]methyl]cyclohexyl]carbamate (85 mg, 156 μmol, 1.0 eq), (2-hydroxyphenyl)boronic acid (53.9 mg, 391 μmol, 2.5 eq), potassium carbonate (64.8 mg, 469 μmol, 3.0 eq) and RuPhos Pd G3 (6.53 mg, 7.81 μmol, 0.05 eq) in a mixture of degassed dioxane (8 mL) and water (0.8 mL) was stirred at 110° C. for 2 h under argon. The reaction mixture was poured in saturated NaHCO₃ and extracted with EtOAc. The organic layers were combined, washed with water and brine. The organic layers were dried over Na₂SO₄ and concentrated in vacuo. The crude material was purified on silica column (heptane/EtOAc 0-100%) to afford the title compound (55 mg, 91 μmol, 58% yield) as an off-white solid. MS (ESI): 602.5 ([M+H]⁺).

### e) N-[(4-aminocyclohexyl)methyl]-4-[3-amino-6-(2-hydroxyphenyl)pyridazin-4-yl]-1-phenyl-piperazine-2-carboxamide

To a cooled (0° C.) solution of tert-butyl (-4-((4-(3-amino-6-(2-hydroxyphenyl)pyridazin-4-yl)-1-phenylpiperazine-2-carboxamido)methyl)cyclohexyl)carbamate (50 mg, 83.1 μmol, 1.0 eq) in DCM (2 mL) was added 4M HCl in dioxane (104 μL, 415 μmol, 5.0 eq). The reaction mixture was allowed to reach room temperature and stirred for 16 h. The reaction mixture was concentrated in vacuo to afford the title compound (40 mg, 74 μmol, 89% yield) as a white solid, hydrochloride salt. MS (ESI): 502.4 ([M+H]⁺).

### f) 4-[3-amino-6-(2-hydroxyphenyl)pyridazin-4-yl]-N-[[4-[[1-[2-(2,6-dioxopiperidin-3-yl)-1,3-dioxoisoindol-5-yl]piperidine-4-carbonyl]amino]cyclohexyl]methyl]-1-phenylpiperazine

Ligase 15 (10 mg, 25.9 μmol, 1.0 eq) was dissolved in dry DMF (200 μL). DIPEA (8.38 mg, 11.3 μL, 64.9 μmol, 2.5 eq) and HATU (11.8 mg, 31.1 μmol, 1.2 eq) were added and the mixture was stirred at room temperature for 10 min. 4-(3-amino-6-(2-hydroxyphenyl)pyridazin-4-yl)-N-((4-aminocyclohexyl)methyl)-1-phenylpiperazine-2-carboxamide, hydrochloride salt (14 mg, 25.9 μmol, 1.0 eq) was added and the mixture was stirred at room temperature for 2 h. The reaction mixture was purified by prep-HPLC to afford the title compound (9.6 mg, 11 μmol, 42% yield) as a light yellow solid. MS (ESI): 869.4 ([M+H]⁺).

### Example 86

### 1-[3-amino-6-(2-hydroxyphenyl)pyridazin-4-yl]-N-[[1-[1-[2-(2,6-dioxo-3-piperidyl)-1,3-dioxo-isoindolin-5-yl]piperidine-4-carbonyl]-4-piperidyl]methyl]-4-phenyl-piperidine-4-carboxamide

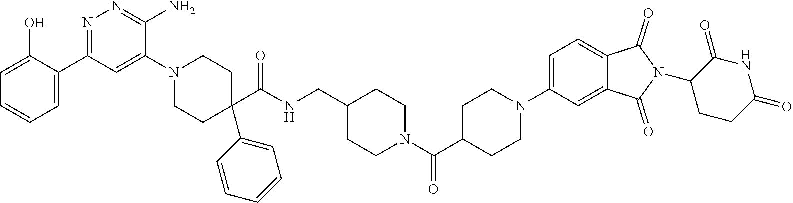 [SMILES: Nc1nnc(-c2ccccc2O)cc1N1CCC(C(=O)NCC2CCN(C(=O)C3CCN(c4ccc5c(c4)C(=O)N(C4CCC(=O)NC4=O)C5=O)CC3)CC2)(c2ccccc2)CC1]

### a) benzyl 4-[(1-tert-butoxycarbonyl-4-piperidyl)methylcarbamoyl]-4-phenyl-piperidine-1-carboxylate

To a solution of 1-((benzyloxy)carbonyl)-4-phenylpiperidine-4-carboxylic acid (300 mg, 884 μmol, 1.0 eq), HATU (420 mg, 1.1 mmol, 1.25 eq) and DIPEA (571 mg, 772 μL, 4.42 mmol, 5.0 eq) in DMF (3 mL) was added tert-butyl 4-(aminomethyl)piperidine-1-carboxylate (227 mg, 224 μL, 1.06 mmol, 1.2 eq). The reaction mixture was stirred at room temperature for 2 h. The reaction mixture was concentrated in vacuo. The crude material was purified on silica column (heptane/EtOAc 0-100%) to afford the title compound (450 mg, 841 μmol, 95% yield) as a yellow oil. MS (ESI): 536.5 ([M+H]⁺).

### b) tert-butyl 4-[[(4-phenylpiperidine-4-carbonyl)amino]methyl]piperidine-1-carboxylate

To a solution of benzyl 4-(((1-(tert-butoxycarbonyl)piperidin-4-yl)methyl)carbamoyl)-4-phenylpiperidine-1-carboxylate (580 mg, 1.08 mmol, 1.0 eq) in methanol (6 mL) was added 10% palladium on charcoal (115 mg, 108 μmol, 0.1 eq). The reaction mixture was vigorously stirred at room temperature for 24 h under H₂ (baloon). The catalyst was collected by filtration, washing with methanol. The filtrate was concentrated to afford the title compound (95 mg, 180 μmol, 91% yield) as a white solid. MS (ESI): 402.4 ([M+H]⁺).

### c) tert-butyl 4-[[[1-(3-amino-6-chloro-pyridazin-4-yl)-4-phenyl-piperidine-4-carbonyl]amino]methyl]piperidine-1-carboxylate

To a solution of 4-bromo-6-chloropyridazin-3-amine (226 mg, 1.08 mmol, 1.1 eq) and tert-butyl 4-((4-phenylpiperidine-4-carboxamido)methyl)piperidine-1-carboxylate (395 mg, 984 μmol, 1.0 eq) in DMSO (4 mL) was added potassium carbonate (680 mg, 4.92 mmol, 5.0 eq) and the reaction mixture was stirred at 110° C. for 16 h. The reaction mixture was cooled to room temperature, poured in saturated NaHCO₃ and extracted with EtOAc. The combined organic layers were washed with water and brine, dried over Na₂SO₄ and concentrated. The crude material was purified on silica column (DCM/MeOH 0-10%) to afford the title compound (365 mg, 691 μmol, 70% yield) as a brown solid. MS (ESI): 529.4 ([M+H]⁺).

### d) tert-butyl 4-[[[1-[3-amino-6-(2-hydroxyphenyl)pyridazin-4-yl]-4-phenyl-piperidine-4-carbonyl]amino]methyl]piperidine-1-carboxylate

A suspension of tert-butyl 4-((1-(3-amino-6-chloropyridazin-4-yl)-4-phenylpiperidine-4-carboxamido)methyl)piperidine-1-carboxylate (360 mg, 680 μmol, 1.0 eq), (2-hydroxyphenyl)boronic acid (235 mg, 1.7 mmol, 2.5 eq), K₂CO₃ (282 mg, 2.04 mmol, 3.0 eq) and RuPhos Pd G3 (28.5 mg, 34 μmol, 0.05 eq) in a mixture of degassed dioxane (6 mL) and water (0.6 ml) was stirred at 110° C. for 3 h under argon. The reaction mixture was poured in saturated NaHCO₃ and extracted with EtOAc. The organic layers were combined, washed with water and brine. The organic layers were dried over Na₂SO₄ and concentrated in vacuo. The crude material was purified on silica column (DCM/MeOH 0-5%) to afford the title compound (300 mg, 512 μmol, 75% yield) as a brown foam. MS (ESI): 476.4 ([M+H]⁺).

### e) 1-[3-amino-6-(2-hydroxyphenyl)pyridazin-4-yl]-4-phenyl-N-(4-piperidylmethyl)piperidine-4-carboxamide

To a cooled (0° C.) solution of tert-butyl 4-((1-(3-amino-6-(2-hydroxyphenyl)pyridazin-4-yl)-4-phenylpiperidine-4-carboxamido)methyl)piperidine-1-carboxylate (300 mg, 511 μmol, 1.0 eq) in DCM (5 mL) was added 4M HCl in dioxane (511 μL, 2.05 mmol, 4.0 eq). The reaction mixture was allowed to reach room temperature and stirred for 16 h. The reaction mixture was filtered and the solid was dried in vacuo to afford the title compound (200 mg, 383 μmol, 75% yield) as a white solid, hydrochloride salt. MS (ESI): 487.4 ([M+H]⁺).

### f) 1-[3-amino-6-(2-hydroxyphenyl)pyridazin-4-yl]-N-[[1-[1-[2-(2,6-dioxo-3-piperidyl)-1,3-dioxo-isoindolin-5-yl]piperidine-4-carbonyl]-4-piperidyl]methyl]-4-phenyl-piperidine-4-carboxamide

Ligase 15 (10 mg, 25.9 μmol, 1.0 eq) was dissolved in dry DMF (200 μL). DIPEA (8.38 mg, 11.3 μL, 64.9 μmol, 2.5 eq) and HATU (11.8 mg, 31.1 μmol, 1.2 eq) were added and the mixture was stirred at room temperature for 10 min. 1-(3-amino-6-(2-hydroxyphenyl)pyridazin-4-yl)-4-phenyl-N-(piperidin-4-ylmethyl)piperidine-4-carboxamide hydrochloride (13.6 mg, 25.9 μmol, 1.0 eq) was added and the mixture was stirred at room temperature for 2 h. The reaction mixture was purified directly by prep-HPLC to afford the title compound (19.6 mg, 20.2 μmol, 78% yield) as a light yellow solid, 2,2,2-trifluoroacetate salt. MS (ESI): 854.6 ([M+H]⁺).

### Example 87

### 4-(3-amino-6-(2-hydroxyphenyl)pyridazin-4-yl)-N-(1-(1-(2-(2,6-dioxopiperidin-3-yl)-1,3-dioxoisoindolin-5-yl)piperidine-4-carbonyl)piperidin-4-yl)-1-phenylpiperazine-2-carboxamide

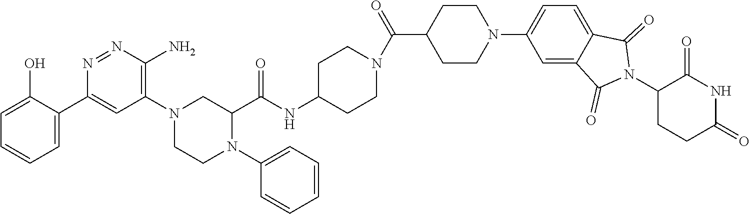 [SMILES: Nc1nnc(-c2ccccc2O)cc1N1CCN(c2ccccc2)C(C(=O)NC2CCN(C(=O)C3CCN(c4ccc5c(c4)C(=O)N(C4CCC(=O)NC4=O)C5=O)CC3)CC2)C1]

The title compound was prepared in analogy to example 53 step f using Ligand 15 as a yellow solid (11.8 mg, 12.4 μmol, 47% yield), (2,2,2-trifluoroacetate) salt. MS (ESI): 841.7 ([M+H]⁺).

### Example 88

### 4-[[1-[1-[9-[4-[4-[3-[3-amino-6-(2-hydroxyphenyl)pyridazin-4-yl]oxy-1-piperidyl]phenyl]piperazin-1-yl]nonyl]triazol-4-yl]-1-methyl-ethyl]amino]-2-(2,6-dioxo-3-piperidyl)isoindoline-1,3-dione

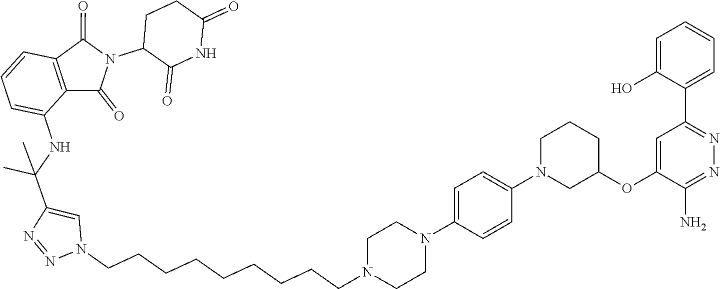 [SMILES: CC(C)(Nc1cccc2c1C(=O)N(C1CCC(=O)NC1=O)C2=O)c1cn(CCCCCCCCCN2CCN(c3ccc(N4CCCC(Oc5cc(-c6ccccc6O)nnc5N)C4)cc3)CC2)nn1]

To a solution of 2-[6-amino-5-[[1-(4-piperazin-1-ylphenyl)-3-piperidyl]oxy]pyridazin-3-yl]phenol (50 mg, 111 μmol) in THF (6 mL) were added Ligase 29 (58 mg, 111 μmol), trimethyl(phenyl)silane (15 mg, 134 μmol) followed by dibutyltin dichloride (34 mg, 111 μmol, 25 μL), and the reaction mixture was heated to 80° C. for 16 h. The reaction mixture was cooled to room temperature, diluted with water and extracted with EtOAc. The organic layer was washed with water, brine solution, dried over anhydrous sodium sulfate, filtered and concentrated under reduced pressure. The crude residue was purified by prep-HPLC to afford the title compound (13.9 mg, 14.2 μmol, 12% yield) as a light yellow solid, trifluoroacetic acid salt. MS (ESI): 952.8 ([M+H]⁺).

### Example 89

### 4-[1-[1-[9-[4-[4-[3-[3-amino-6-(2-hydroxyphenyl)pyridazin-4-yl]oxy-1-piperidyl]phenyl]piperazin-1-yl]nonyl]triazol-4-yl]ethylamino]-2-(2,6-dioxo-3-piperidyl)isoindoline-1,3-dione

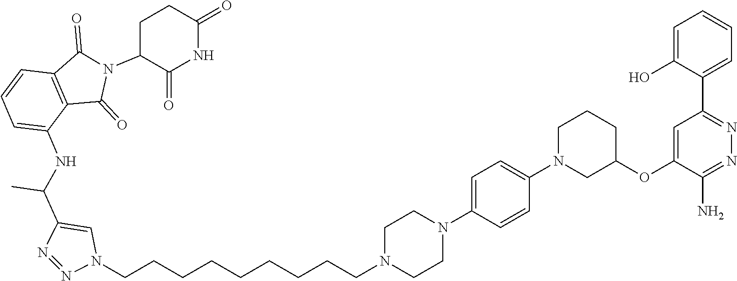 [SMILES: CC(Nc1cccc2c1C(=O)N(C1CCC(=O)NC1=O)C2=O)c1cn(CCCCCCCCCN2CCN(c3ccc(N4CCCC(Oc5cc(-c6ccccc6O)nnc5N)C4)cc3)CC2)nn1]

To a solution of 2-[6-amino-5-[[1-(4-piperazin-1-ylphenyl)-3-piperidyl]oxy]pyridazin-3-yl]phenol (40 mg, 89 μmol) in THF (6 mL) were added Ligase 30 (45 mg, 89 μmol), Trimethyl(phenyl)silane (12 mg, 107 μmol) followed by dibutyltin dichloride (27 mg, 90 μmol, 20 μL), and the reaction mixture was heated to 80° C. for 16 h. The reaction mixture was cooled to room temperature, diluted with water and extracted with EtOAc. The organic layer was washed with water, brine solution, dried over anhydrous sodium sulfate, filtered and concentrated under reduced pressure. The crude residue was purified by prep-HPLC to afford the title compound (16.8 mg, 17 μmol, 19% yield) as a light yellow solid, trifluoroacetic acid salt. MS (ESI): 938.8 ([M+H]⁺).

### Example 90

### 4-[[1-[1-[9-[4-[4-[3-[3-amino-6-(2-hydroxyphenyl)pyridazin-4-yl]oxy-1-piperidyl]phenyl]piperazin-1-yl]nonyl]triazol-4-yl]cyclopropyl]amino]-2-(2,6-dioxo-3-piperidyl)isoindoline-1,3-dione

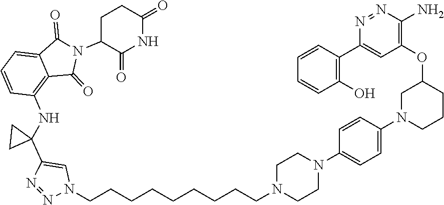 [SMILES: Nc1nnc(-c2ccccc2O)cc1OC1CCCN(c2ccc(N3CCN(CCCCCCCCCn4cc(C5(Nc6cccc7c6C(=O)N(C6CCC(=O)NC6=O)C7=O)CC5)nn4)CC3)cc2)C1]

To a solution of 2-[6-amino-5-[[1-(4-piperazin-1-ylphenyl)-3-piperidyl]oxy]pyridazin-3-yl]phenol (50 mg, 89 μmol, TFA salt) and Ligase 31 (122 mg, 89 μmol) in THF (5 mL) was added dibutyltin dichloride (27 mg, 89 μmol, 19 μL) followed by the addition of trimethyl(phenyl)silane (12 mg, 107.23 μmol) and the reaction mixture was heated to 80° C. for 16 h. The reaction mixture was cooled to room temperature, diluted with water and extracted with EtOAc. The organic layer was washed with water, brine solution, dried over anhydrous sodium sulfate, filtered and concentrated under reduced pressure. The crude residue was purified by prep-HPLC to afford the title compound (4.42 mg, 4.4 μmol, 5% yield) as a light green solid, trifluoroacetic acid salt. MS (ESI): 951.3 ([M+H]⁺).

### Example 91

### N-[1-[3-amino-6-(2-hydroxyphenyl)pyridazin-4-yl]-4-phenyl-4-piperidyl]-4-[4-[4-[(2,6-dioxo-3-piperidyl)amino]phenyl]-1-piperidyl]butanamide

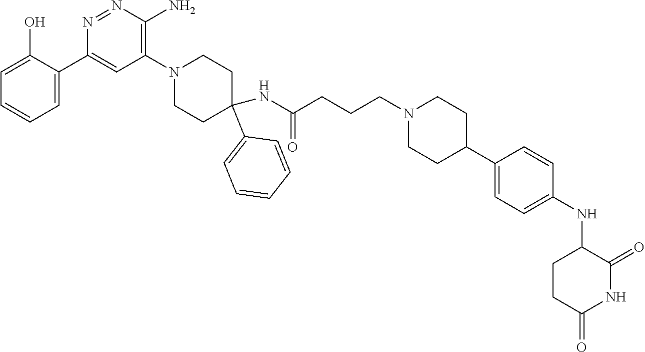 [SMILES: Nc1nnc(-c2ccccc2O)cc1N1CCC(NC(=O)CCCN2CCC(c3ccc(NC4CCC(=O)NC4=O)cc3)CC2)(c2ccccc2)CC1]

### a) tert-butyl (1-(3-amino-6-chloropyridazin-4-yl)-4-phenylpiperidin-4-yl)carbamate

4-bromo-6-chloropyridazin-3-amine (870 mg, 4.17 mmol, 1.0 eq) was combined with tert-butyl (4-phenylpiperidin-4-yl)carbamate (1.38 g, 5.01 mmol, 1.2 eq) and K₂CO₃ (1.44 g, 10.4 mmol, 2.5 eq) in DMA (9 mL) in a sealed tube. The reaction was heated to 120° C. and stirred for 2 h. The solvent was evaporated. The crude residue was purified on silica column (DCM/MeOH 0-20%) as eluent to afford the title compound (1.34 g, 3.33 mmol, 79% yield) as an off-white solid. MS (ESI): 404.4 ([M+H]⁺).

### b) tert-butyl (1-(3-amino-6-(2-hydroxyphenyl)pyridazin-4-yl)-4-phenylpiperidin-4-yl)carbamate

Tert-butyl (1-(3-amino-6-chloropyridazin-4-yl)-4-phenylpiperidin-4-yl)carbamate (1.34 g, 3.32 mmol, 1.0 eq) was combined with (2-hydroxyphenyl)boronic acid (915 mg, 6.64 mmol, 2.0 eq), RuPhos Pd G3 (139 mg, 166 μmol, 0.05 eq) and potassium carbonate (1.15 g, 8.29 mmol, 2.5 eq) in 1,4-dioxane (12 mL) and Water (1.2 mL). The reaction was purged with argon, heated to 120° C. and was stirred for 2 h. EtOAc was added and the mixture was extracted with water. A light yellow precipitate formed in the organic layer. The aqueous layer was removed and DCM/methanol (9:1) was added to the organic layer until the precipitate was dissolved. The organic layer was then dried over magnesium sulfate, filtered and evaporated. The crude residue was purified on silica column (DCM/MeOH 0-10%) as eluent to afford the title compound (1.32 g, 2.8 mmol, 86% yield) as an orange solid. MS (ESI): 462.4 ([M+H]⁺).

### c) 2-(6-amino-5-(4-amino-4-phenylpiperidin-1-yl)pyridazin-3-yl)phenol

4M HCl in dioxane (3.57 mL, 14.3 mmol, 5.0 eq) was added to a purple solution of tert-butyl (1-(3-amino-6-(2-hydroxyphenyl)pyridazin-4-yl)-4-phenylpiperidin-4-yl)carbamate (1.32 g, 2.86 mmol, 1.0 eq) in DCM (13 mL) at room temperature. During the addition, the solution slowly turned orange and then yellow, until finally a yellow precipitate formed. Stirring was continued overnight. Aqueous saturated NaHCO₃ was added until pH=8 and the mixture was extracted with EtOAc. The organic layer was dried over magnesium sulfate, filtered and evaporated to afford the title compound (1.02 g, 2.8 mmol, 99% yield) as an orange solid. MS (ESI): 363.4 ([M+H]⁺).

### d) N-(1-(3-amino-6-(2-hydroxyphenyl)pyridazin-4-yl)-4-phenylpiperidin-4-yl)-4-(4-(4-((2,6-dioxopiperidin-3-yl)amino)phenyl)piperidin-1-yl)butanamide

2-(6-amino-5-(4-amino-4-phenylpiperidin-1-yl)pyridazin-3-yl)phenol (60 mg, 166 μmol, 1.0 eq) was combined with Ligase 52 (74.8 mg, 183 μmol, 1.1 eq), HATU (126 mg, 332 μmol, 2.0 eq) and DIPEA (107 mg, 145 μL, 830 μmol, 5.0 eq) in DMF (600 μL). The brown solution was stirred at room temperature for 2 h and was then purified by prep-HPLC to afford the title compound (40.2 mg, 42.5 μmol, 25% yield) as a white salt, bis-(2,2,2-trifluoroacetic acid)salt. MS (ESI): 717.3866 ([M+H]⁺).

### Example 92

### rac-3-[4-[1-[1-[[3-[3-[3-amino-6-(2-hydroxyphenyl)pyridazin-4-yl]-3,8-diazabicyclo[3.2.1]octan-8-yl]phenyl]methyl]piperidine-4-carbonyl]-4-piperidyl]phenoxy]piperidine-2,6-dione

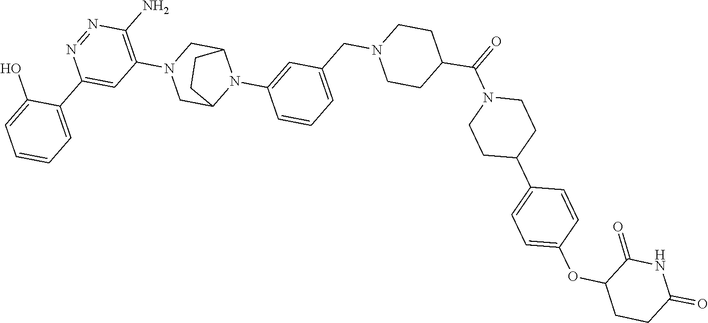 [SMILES: Nc1nnc(-c2ccccc2O)cc1N1CC2CCC(C1)N2c1cccc(CN2CCC(C(=O)N3CCC(c4ccc(OC5CCC(=O)NC5=O)cc4)CC3)CC2)c1]

### a) tert-butyl 8-[3-[(4-methoxycarbonyl-1-piperidyl)methyl]phenyl]-3,8-diazabicyclo[3.2.1]octane-3-carboxylate

To a stirred suspension of tert-butyl 3,8-diazabicyclo[3.2.1]octane-3-carboxylate (CAS 201162-53-0, 785 mg, 3.7 mmol, 1.1 eq) and methyl 1-(3-bromobenzyl)piperidine-4-carboxylate (CAS 1057273-30-9, 1.05 g, 3.36 mmol, 1.0 eq) in t-BuOH (8 mL) was added K₂CO₃ (930 mg, 6.73 mmol, 2.0 eq). The reaction mixture was degassed with argon for 10 min. RuPhos Pd G3 (281 mg, 336 μmol, 0.1 eq) was added. The reaction mixture was stirred at 115° C. for 7 h. The reaction mixture was then cooled to room temperature, poured into EtOAc/THF (2:1), and washed sequentially with water and with brine. The organic layer was dried over Na₂SO₄ and concentrated in vacuo. The crude material was purified on silica column (heptane/EtOAc 0-80%) to afford the title compound (1.04 g, 2.34 mmol, 70% yield) as a yellow solid. MS (ESI): 444.4 ([M+H]⁺).

### b) methyl 1-[[3-(3,8-diazabicyclo[3.2.1]octan-8-yl)phenyl]methyl]piperidine-4-carboxylate

To a stirred solution of tert-butyl 8-[3-[(4-methoxycarbonyl-1-piperidyl)methyl]phenyl]-3,8-diazabicyclo[3.2.1]octane-3-carboxylate (1.04 g, 2.34 mmol, 1.0 eq) in DCM (8 mL) at room temperature was added TFA (5.35 g, 3.61 ml, 46.9 mmol, 20.0 eq). The reaction mixture was stirred at room temperature for 2 h. The reaction mixture was poured in EtOAc/THF (2:1) and washed with 0.5 N aqueous NaOH and with brine. The organic layer was dried over Na₂SO₄ and concentrated in vacuo to afford the title compound (0.96 g, 2.35 mmol, 100% yield). MS (ESI): 344.3 ([M+H]⁺).

### c) methyl 1-[[3-[3-(3-amino-6-chloro-pyridazin-4-yl)-3,8-diazabicyclo[3.2.1]octan-8-yl]phenyl]methyl]piperidine-4-carboxylate

To a stirred solution of methyl 1-[[3-(3,8-diazabicyclo[3.2.1]octan-8-yl)phenyl]methyl]piperidine-4-carboxylate (0.96 g, 2.35 mmol, 1.0 eq) and 4-bromo-6-chloropyridazin-3-amine (587 mg, 2.82 mmol, 1.2 eq) in DMSO (4 mL) at room temperature was added K₂CO₃ (1.62 g, 11.7 mmol, 5.0 eq). The reaction mixture was stirred at 110° C. for 2.5 h. The reaction mixture was poured in EtOAc/THF (2:1) and washed sequentially with water and with brine. The organic layer was dried over Na₂SO₄ and concentrated in vacuo. The crude material was purified on silica column (DCM/MeOH 0-10%) to afford the title compound (819 mg, 1.74 mmol, 74% yield) as a light brown foam. MS (ESI): 471.4 (³⁵Cl[M+H]⁺).

### d) methyl 1-[[3-[3-[3-amino-6-(2-hydroxyphenyl)pyridazin-4-yl]-3,8-diazabicyclo[3.2.1]octan-8-yl]phenyl]methyl]piperidine-4-carboxylate

To a stirred slurry mixture of methyl 1-[[3-[3-(3-amino-6-chloro-pyridazin-4-yl)-3,8-diazabicyclo[3.2.1]octan-8-yl]phenyl]methyl]piperidine-4-carboxylate (0.81 g, 1.72 mmol, 1.0 eq) and (2-hydroxyphenyl)boronic acid (593 mg, 4.3 mmol, 2.5 eq) in dioxane (30 mL) and water (3 mL) was added K₂CO₃ (832 mg, 6.02 mmol, 3.5 eq). The reaction mixture was degassed with argon for 10 min. RuPhos Pd G3 (144 mg, 172 μmol, 0.1 eq) was added. The reaction mixture was stirred at 90° C. for 2 h. The reaction mixture was poured into EtOAc/THF (1:1) and washed sequentially with water and brine. The organic layer was dried over Na₂SO₄ and concentrated in vacuo. The crude material was purified on silica column (DCM/MeOH 0-5%) then on amine modified silica column (DCM/MeOH 0-5%) to afford the title compound (646 mg, 1.22 mmol, 71% yield) as a yellow solid. MS (ESI): 529.5 ([M+H]⁺).

### e) 1-[[3-[3-[3-amino-6-(2-hydroxyphenyl)pyridazin-4-yl]-3,8-diazabicyclo[3.2.1]octan-8-yl]phenyl]methyl]piperidine-4-carboxylic acid

To a stirred solution of methyl 1-[[3-[3-[3-amino-6-(2-hydroxyphenyl)pyridazin-4-yl]-3,8-diazabicyclo[3.2.1]octan-8-yl]phenyl]methyl]piperidine-4-carboxylate (0.64 g, 1.21 mmol, 1.0 eq) in methanol (12 mL) and THF (6 mL) was added 1 M aqueous NaOH (3.63 mL, 3.63 mmol, 3.0 eq). The reaction mixture was stirred at 40° C. for 2 h. 2 M aqueous HCl was added dropwise until the mixture was pH=3. The reaction mixture was then concentrated in vacuo. The reaction mixture was dissolved in water/acetonitrile and lyophilised to afford the title compound (0.94 g, 1.21 mmol, 100% yield) as a yellow solid. MS (ESI): 515.4 ([M+H]⁺).

### f) rac-3-[4-[1-[1-[[3-[3-[3-amino-6-(2-hydroxyphenyl)pyridazin-4-yl]-3,8-diazabicyclo[3.2.1]octan-8-yl]phenyl]methyl]piperidine-4-carbonyl]-4-piperidyl]phenoxy]piperidine-2,6-dione

To a stirred solution of 1-[[3-[3-[3-amino-6-(2-hydroxyphenyl)pyridazin-4-yl]-3,8-diazabicyclo[3.2.1]octan-8-yl]phenyl]methyl]piperidine-4-carboxylic acid (60 mg, 76.9 μmol, 1.0 eq), Ligase 38 (25 mg, 76.9 μmol, 1.0 eq) and DIPEA (49.7 mg, 67.2 μL, 385 μmol, 5.0 eq) in DMSO (0.6 mL) at room temperature was added HATU (58.5 mg, 154 μmol, 2.0 eq). The reaction mixture was stirred at room temperature for 1 h. The crude material was purified by prep-HPLC. The product was lyophilised to afford the title compound (29 mg, 28.6 μmol, 37% yield) as a yellow solid, bis-(2,2,2-trifluoroacetic acid)salt. MS (ESI): 785.4 ([M+H]⁺).

### Example 93

### rac-3-[4-[1-[1-[[3-[3-[3-amino-6-(2-hydroxyphenyl)pyridazin-4-yl]-3,8-diazabicyclo[3.2.1]octan-8-yl]phenyl]methyl]piperidine-4-carbonyl]-4-piperidyl]anilino]piperidine-2,6-dione

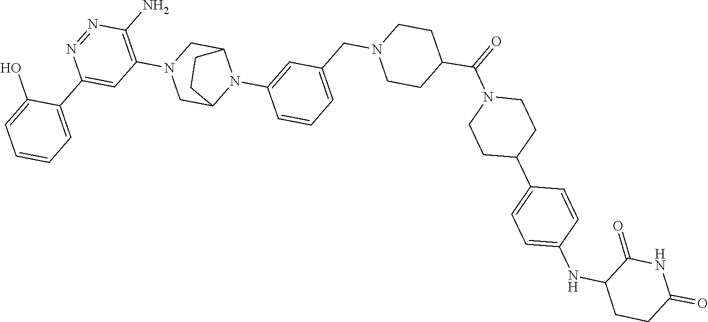 [SMILES: Nc1nnc(-c2ccccc2O)cc1N1CC2CCC(C1)N2c1cccc(CN2CCC(C(=O)N3CCC(c4ccc(NC5CCC(=O)NC5=O)cc4)CC3)CC2)c1]

To a stirred solution of 1-[[3-[3-[3-amino-6-(2-hydroxyphenyl)pyridazin-4-yl]-3,8-diazabicyclo[3.2.1]octan-8-yl]phenyl]methyl]piperidine-4-carboxylic acid (55 mg, 70.5 μmol, 1.0 eq), Ligase 50 (22.8 mg, 70.5 μmol, 1.0 eq) and DIPEA (45.6 mg, 61.6 μL, 353 μmol, 5.0 eq) in DMSO at room temperature was added HATU (53.6 mg, 141 μmol, 2.0 eq). The reaction mixture was stirred for 1 h at room temperature. The crude material was purified by prep-HPLC to afford the title compound (30 mg, 29.6 μmol, 42% yield) as a yellow solid, bis-(2,2,2-trifluoroacetic acid)salt. MS (ESI): 784.4 ([M+H]⁺).

### Example 94

### rac-3-[4-[4-[1-[[3-[3-[3-amino-6-(2-hydroxyphenyl)pyridazin-4-yl]-3,8-diazabicyclo[3.2.1]octan-8-yl]phenyl]methyl]piperidine-4-carbonyl]piperazin-1-yl]phenoxy]piperidine-2,6-dione

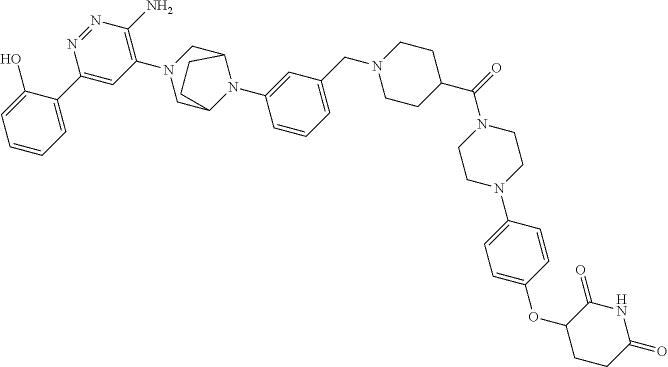 [SMILES: Nc1nnc(-c2ccccc2O)cc1N1CC2CCC(C1)N2c1cccc(CN2CCC(C(=O)N3CCN(c4ccc(OC5CCC(=O)NC5=O)cc4)CC3)CC2)c1]

To a stirred solution of 1-[[3-[3-[3-amino-6-(2-hydroxyphenyl)pyridazin-4-yl]-3,8-diazabicyclo[3.2.1]octan-8-yl]phenyl]methyl]piperidine-4-carboxylic acid (60 mg, 76.9 μmol, 1.0 eq), Ligase 41 (25.1 mg, 76.9 μmol, 1.0 eq) and DIPEA (49.7 mg, 67.2 μL, 385 μmol, 5.0 eq) in DMSO (0.6 mL) at room temperature was added HATU (58.5 mg, 154 μmol, 2.0 eq). The reaction mixture was stirred at room temperature for 1 h. The crude material was purified by prep-HPLC. The product was lyophilised to afford the title compound (16 mg, 15.8 μmol, 21% yield) as a yellow solid, bis-(2,2,2-trifluoroacetic acid)salt. MS (ESI): 786.4 ([M+H]⁺).

### Example 95

### rac-5-[4-[1-[[3-[3-[3-amino-6-(2-hydroxyphenyl)pyridazin-4-yl]-3,8-diazabicyclo[3.2.1]octan-8-yl]phenyl]methyl]piperidine-4-carbonyl]piperazin-1-yl]-2-(2,6-dioxo-3-piperidyl)isoindoline-1,3-dione

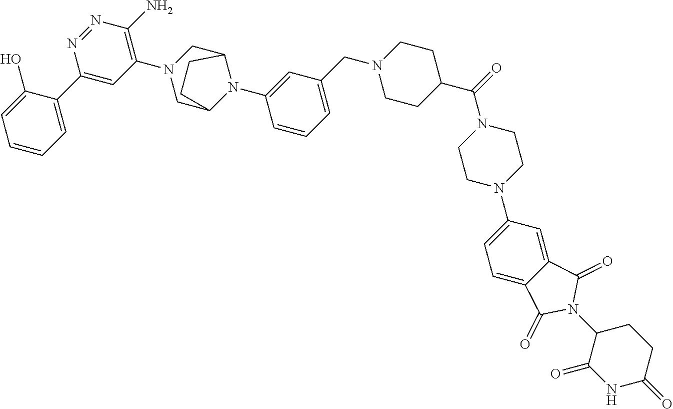 [SMILES: Nc1nnc(-c2ccccc2O)cc1N1CC2CCC(C1)N2c1cccc(CN2CCC(C(=O)N3CCN(c4ccc5c(c4)C(=O)N(C4CCC(=O)NC4=O)C5=O)CC3)CC2)c1]

To a stirred solution of 1-[[3-[3-[3-amino-6-(2-hydroxyphenyl)pyridazin-4-yl]-3,8-diazabicyclo[3.2.1]octan-8-yl]phenyl]methyl]piperidine-4-carboxylic acid (60 mg, 76.9 μmol, 1.0 eq), Ligase 37 (29.1 mg, 76.9 μmol, 1.0 eq) and DIPEA (49.7 mg, 67.2 μL, 385 μmol, 5.0 eq) in DMSO (0.6 mL) at room temperature was added HATU (58.5 mg, 154 μmol, 2.0 eq). The reaction mixture was stirred at room temperature for 1 h. The crude material was purified by prep-HPLC to afford the title compound (36 mg, 33.7 μmol, 44% yield) as a yellow solid, bis-(2,2,2-trifluoroacetic acid)salt. MS (ESI): 839.4 ([M+H]⁺).

### Example 96

### rac-5-[[1-[1-[[3-[3-[3-amino-6-(2-hydroxyphenyl)pyridazin-4-yl]-3,8-diazabicyclo[3.2.1]octan-8-yl]phenyl]methyl]piperidine-4-carbonyl]-4-piperidyl]oxy]-2-(2,6-dioxo-3-piperidyl)isoindoline-1,3-dione

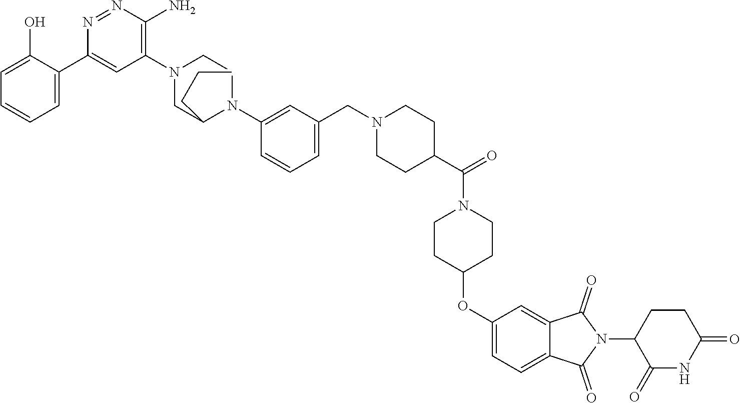

To a stirred solution of 1-[[3-[3-[3-amino-6-(2-hydroxyphenyl)pyridazin-4-yl]-3,8-diazabicyclo[3.2.1]octan-8-yl]phenyl]methyl]piperidine-4-carboxylic acid (60 mg, 76.9 μmol, 1.0 eq), Ligase 8 (30.3 mg, 76.9 μmol, 1.0 eq, HCl salt) and DIPEA (49.7 mg, 67.2 μL, 385 μmol, 5.0 eq) in DMSO (0.6 mL) at room temperature was added HATU (58.5 mg, 154 μmol, 2.0 eq). The reaction mixture was stirred at room temperature for 1 h. The crude material was purified by prep-HPLC to afford the title compound (49 mg, 45.3 μmol, 59% yield) as a yellow salt, bis-(2,2,2-trifluoroacetic acid)salt. MS (ESI): 854.4 ([M+H]⁺).

### Example 97

### N-[3-[3-amino-6-(2-hydroxyphenyl)pyridazin-4-yl]oxy-2-phenyl-propyl]-1-[2-(2,6-dioxo-3-piperidyl)-1,3-dioxo-isoindolin-5-yl]piperidine-4-carboxamide (racemic mixture of diastereomers)

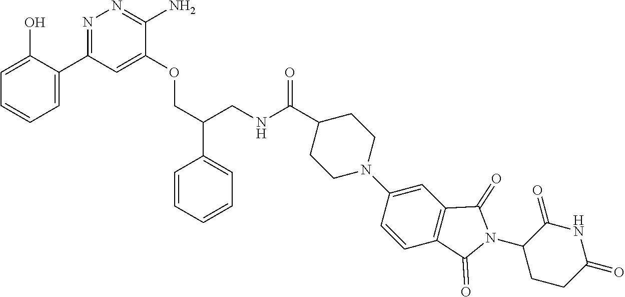 [SMILES: Nc1nnc(-c2ccccc2O)cc1OCC(CNC(=O)C1CCN(c2ccc3c(c2)C(=O)N(C2CCC(=O)NC2=O)C3=O)CC1)c1ccccc1]

To a stirred solution of 2-[6-amino-5-(3-amino-2-phenyl-propoxy)pyridazin-3-yl]phenol hydrochloride (30 mg, 80.5 μmol, 1.0 eq), Ligase 15 (31 mg, 80.5 μmol, 1.0 eq) and HATU (61.2 mg, 161 μmol, 2.0 eq) in DMSO (0.8 mL) at room temperature was added DIPEA (31.2 mg, 42.2 μL, 241 μmol, 3.0 eq). The reaction mixture was stirred for 3 h. The purification was done by prep-HPLC to afford the title compound (23.6 mg, 28.9 μmol, 36% yield) as a yellow solid, 2,2,2-trifluoroacetate salt. MS (ESI): 704.5 ([M+H]⁺).

### Example 98

### N-[3-[3-amino-6-(2-hydroxyphenyl)pyridazin-4-yl]oxy-2-phenyl-propyl]-2-[4-[4-[(2,6-dioxo-3-piperidyl)oxy]phenyl]-1-piperidyl]acetamide;2,2,2-trifluoroacetic acid (racemic mixture of diastereomers)

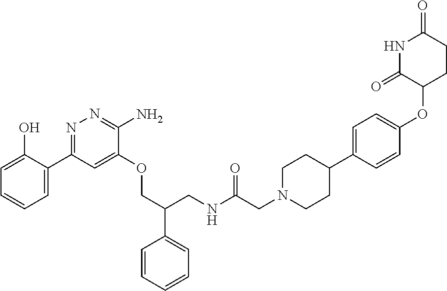 [SMILES: Nc1nnc(-c2ccccc2O)cc1OCC(CNC(=O)CN1CCC(c2ccc(OC3CCC(=O)NC3=O)cc2)CC1)c1ccccc1]

To a stirred solution of 2-[6-amino-5-(3-amino-2-phenyl-propoxy)pyridazin-3-yl]phenol hydrochloride (30 mg, 80.5 μmol, 1.0 eq), Ligase 35 (30.8 mg, 80.5 μmol, 1.0 eq) and HATU (61.2 mg, 161 μmol, 2.0 eq) in dimethyl sulfoxide (0.8 mL) at room temperature was added DIPEA (41.6 mg, 56.2 μL, 322 μmol, 4.0 eq). The reaction mixture was stirred for 3 h. The purification was done by prep-HPLC to afford the title compound (racemic mixture of diastereomers) (11.5 mg, 14.8 μmol, 18% yield) as a white solid, 2,2,2-trifluoroacetate salt. MS (ESI): 665.5 ([M+H]⁺).

### Example 99

### rac-5-[2-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-2-oxo-ethoxy]-2-(2,6-dioxo-3-piperidyl)isoindoline-1,3-dione

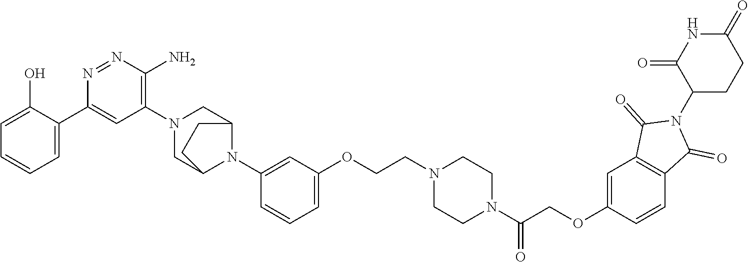 [SMILES: Nc1nnc(-c2ccccc2O)cc1N1CC2CCC(C1)N2c1cccc(OCCN2CCN(C(=O)COc3ccc4c(c3)C(=O)N(C3CCC(=O)NC3=O)C4=O)CC2)c1]

To a stirred solution of 2-[6-amino-5-[8-[3-(2-piperazin-1-ylethoxy)phenyl]-3,8-diazabicyclo[3.2.1]octan-3-yl]pyridazin-3-yl]phenol 44.4 mg, 88.5 μmol, 1.0 eq), Ligase 48 (29.4 mg, 88.5 μmol, 1.0 eq) and DIPEA (34.3 mg, 46.4 μL, 265 μmol, 3.0 eq) in DMSO (0.5 mL) at room temperature was added HATU (67.3 mg, 177 μmol, 2.0 eq). The reaction mixture was stirred for 3 h. The reaction mixture was partitioned between EtOAc/THF (1:1) and saturated aqueous bicarbonate solution. The layers were separated. The aqueous layer was extracted with EtOAc. The combined organic layers were washed with brine, dried over anhydrous sodium sulfate, and concentrated in vacuo. The crude material was purified on silica column (DCM/MeOH 0-10%) to afford the title compound (35.6 mg, 43.6 μmol, 49% yield) as an off-white solid. MS (ESI): 816.7 ([M+H]⁺).

### Example 100

### 1-(3-amino-6-(2-hydroxyphenyl)pyridazin-4-yl)-N-((1-((1-(2-(2,6-dioxopiperidin-3-yl)-1,3-dioxoisoindolin-5-yl)piperidin-4-yl)methyl)piperidin-4-yl)methyl)-4-phenylpiperidine-4-carboxamide

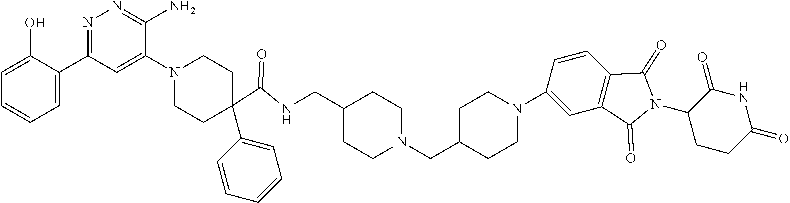 [SMILES: Nc1nnc(-c2ccccc2O)cc1N1CCC(C(=O)NCC2CCN(CC3CCN(c4ccc5c(c4)C(=O)N(C4CCC(=O)NC4=O)C5=O)CC3)CC2)(c2ccccc2)CC1]

1-(3-amino-6-(2-hydroxyphenyl)pyridazin-4-yl)-4-phenyl-N-(piperidin-4-ylmethyl)piperidine-4-carboxamide, hydrochloride salt (9 mg, 18.5 μmol, 1.0 eq) and Ligase 33 (8.03 mg, 18.5 μmol, 1.0 eq) were stirred in DMSO (400 μL) for 72 h. The reaction mixture was purified directly by prep-HPLC to afford the title compound (8.1 mg, 8.49 μmol, 45% yield) as a light brown solid, (2,2,2-trifluoroacetate) salt. MS (ESI): 840.7 ([M+H]⁺).

### Example 101

### 1-(3-amino-6-(2-hydroxyphenyl)pyridazin-4-yl)-N-((1-(2-(4-(4-((2,6-dioxopiperidin-3-yl)oxy)phenyl)piperidin-1-yl)acetyl)piperidin-4-yl)methyl)-4-phenylpiperidine-4-carboxamide

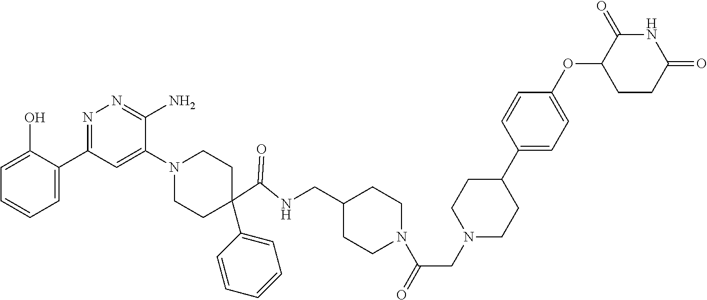 [SMILES: Nc1nnc(-c2ccccc2O)cc1N1CCC(C(=O)NCC2CCN(C(=O)CN3CCC(c4ccc(OC5CCC(=O)NC5=O)cc4)CC3)CC2)(c2ccccc2)CC1]

The title compound was prepared in analogy to example 86 step f using Ligase 35 as a light yellow solid (25.9 mg, 27.9 μmol, 72% yield), (2,2,2-trifluoroacetate) salt. MS (ESI): 815.6 ([M+H]⁺).

### Example 102

### 1-[3-amino-6-(2-hydroxyphenyl)pyridazin-4-yl]-N-[[1-[2-[4-[4-[(2,6-dioxopiperidin-3-yl)amino]phenyl]piperidin-1-yl]acetyl]piperidin-4-yl]methyl]-4-phenylpiperidine-4-carboxamide

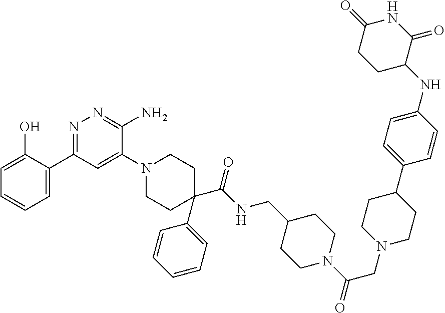 [SMILES: Nc1nnc(-c2ccccc2O)cc1N1CCC(C(=O)NCC2CCN(C(=O)CN3CCC(c4ccc(NC5CCC(=O)NC5=O)cc4)CC3)CC2)(c2ccccc2)CC1]

The title compound was prepared in analogy to example 86 step f using Ligase 51 as a light yellow solid (25 mg, 25.6 μmol, 66% yield), (2,2,2-trifluoroacetate) salt. MS (ESI): 814.7 ([M+H]⁺).

### Example 103

### 1-(3-amino-6-(2-hydroxyphenyl)pyridazin-4-yl)-N-((1-(2-(1-(2-(2,6-dioxopiperidin-3-yl)-1,3-dioxoisoindolin-5-yl)piperidin-4-yl)acetyl)piperidin-4-yl)methyl)-4-phenylpiperidine-4-carboxamide

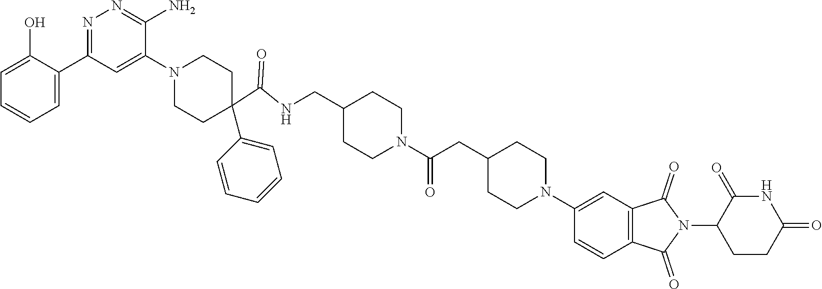 [SMILES: Nc1nnc(-c2ccccc2O)cc1N1CCC(C(=O)NCC2CCN(C(=O)CC3CCN(c4ccc5c(c4)C(=O)N(C4CCC(=O)NC4=O)C5=O)CC3)CC2)(c2ccccc2)CC1]

The title compound was prepared in analogy to example 86 step f using Ligase 24 as a light yellow solid (19.4 mg, 18.8 μmol, 49% yield), (2,2,2-trifluoroacetate) salt. MS (ESI): 868.7 ([M+H]⁺).

### Example 104

### 1-(3-amino-6-(2-hydroxyphenyl)pyridazin-4-yl)-N-((1-(4-(4-(4-((2,6-dioxopiperidin-3-yl)oxy)phenyl)piperidin-1-yl)butanoyl)piperidin-4-yl)methyl)-4-phenylpiperidine-4-carboxamide

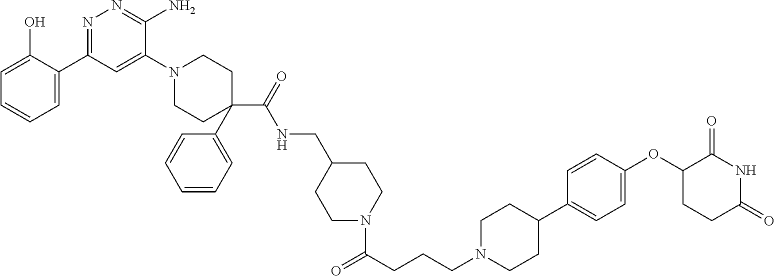 [SMILES: Nc1nnc(-c2ccccc2O)cc1N1CCC(C(=O)NCC2CCN(C(=O)CCCN3CCC(c4ccc(OC5CCC(=O)NC5=O)cc4)CC3)CC2)(c2ccccc2)CC1]

The title compound was prepared in analogy to example 86 step f using ligase 39 as a light yellow solid (18.9 mg, 18.8 μmol, 49% yield), (2,2,2-trifluoroacetate) salt. MS (ESI): 843.7 ([M+H]⁺).

### Example 105

### 1-(3-amino-6-(2-hydroxyphenyl)pyridazin-4-yl)-N-((1-(4-(4-(4-((2,6-dioxopiperidin-3-yl)amino)phenyl)piperidin-1-yl)butanoyl)piperidin-4-yl)methyl)-4-phenylpiperidine-4-carboxamide

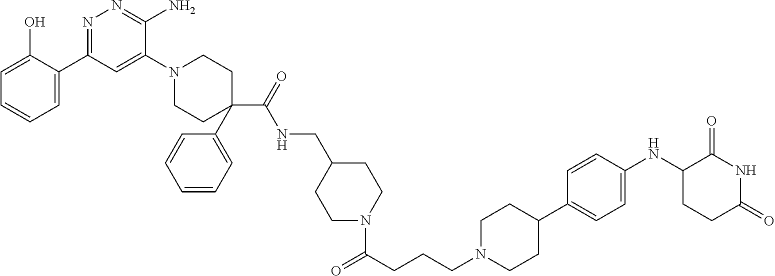 [SMILES: Nc1nnc(-c2ccccc2O)cc1N1CCC(C(=O)NCC2CCN(C(=O)CCCN3CCC(c4ccc(NC5CCC(=O)NC5=O)cc4)CC3)CC2)(c2ccccc2)CC1]

The title compound was prepared in analogy to example 86 step f using Ligase 52 as a light yellow solid 52 (15 mg, 14.9 μmol, 39% yield), (2,2,2-trifluoroacetate) salt. MS (ESI): 842.8 ([M+H]⁺).

### Example 106

### rac-1-[[3-[3-[3-amino-6-(2-hydroxyphenyl)pyridazin-4-yl]-3,8-diazabicyclo[3.2.1]octan-8-yl]phenyl]methyl]-N-[1-[2-(2,6-dioxo-3-piperidyl)-1,3-dioxo-isoindolin-5-yl]-4-piperidyl]piperidine-4-carboxamide

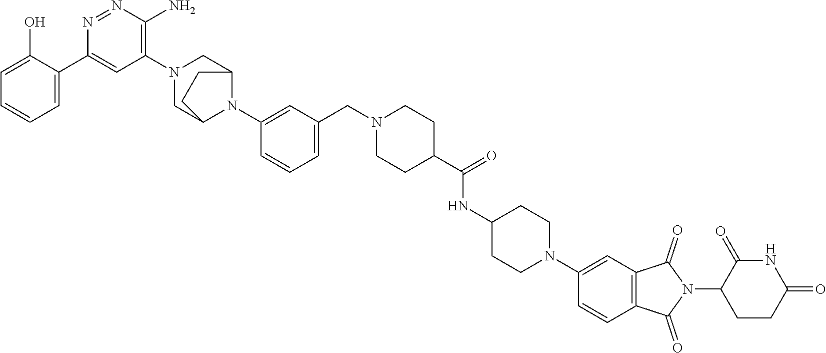 [SMILES: Nc1nnc(-c2ccccc2O)cc1N1CC2CCC(C1)N2c1cccc(CN2CCC(C(=O)NC3CCN(c4ccc5c(c4)C(=O)N(C4CCC(=O)NC4=O)C5=O)CC3)CC2)c1]

To a stirred solution of 1-[[3-[3-[3-amino-6-(2-hydroxyphenyl)pyridazin-4-yl]-3,8-diazabicyclo[3.2.1]octan-8-yl]phenyl]methyl]piperidine-4-carboxylic acid (60 mg, 76.9 μmol, 1.0 eq), Ligase 55 (27.4 mg, 76.9 μmol, 1.0 eq) and DIPEA (49.7 mg, 67.2 μL, 385 μmol, 5.0 eq) in DMSO (0.6 mL) at room temperature was added HATU (58.5 mg, 154 μmol, 2.0 eq). The reaction mixture was stirred at room temperature for 1 h. The crude material was purified by prep-HPLC. The product was lyophilised to afford the title compound (15 mg, 13.9 μmol, 18% yield) as a yellow solid, bis-(2,2,2-trifluoroacetic acid)salt. MS (ESI): 853.4 ([M+H]⁺).

### Example 107

### rac-5-[4-[4-[5-[3-[3-amino-6-(2-hydroxyphenyl)pyridazin-4-yl]-3,8-diazabicyclo[3.2.1]octan-8-yl]-2-(trifluoromethyl)benzoyl]piperazine-1-carbonyl]-1-piperidyl]-2-(2,6-dioxo-3-piperidyl)isoindoline-1,3-dione

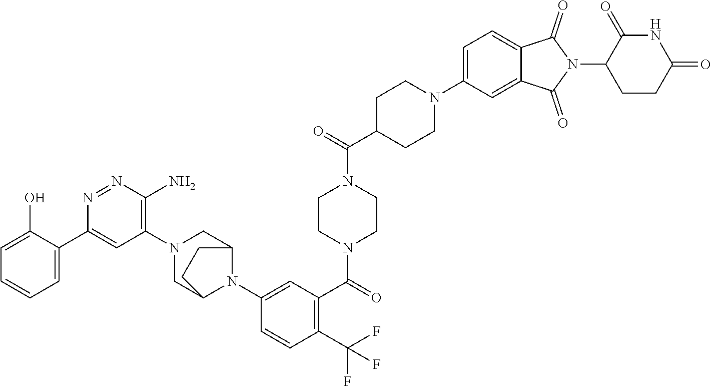 [SMILES: Nc1nnc(-c2ccccc2O)cc1N1CC2CCC(C1)N2c1ccc(C(F)(F)F)c(C(=O)N2CCN(C(=O)C3CCN(c4ccc5c(c4)C(=O)N(C4CCC(=O)NC4=O)C5=O)CC3)CC2)c1]

### a) benzyl 4-[5-fluoro-2-(trifluoromethyl)benzoyl]piperazine-1-carboxylate

To a stirred solution of 5-fluoro-2-(trifluoromethyl)benzoic acid (500 mg, 2.4 mmol, 1.0 eq), benzyl piperazine-1-carboxylate (688 mg, 602 μL, 3.12 mmol, 1.3 eq) and HATU (1.92 g, 5.05 mmol, 2.1 eq) in dimethyl sulfoxide (3 mL) at room temperature was added DIPEA (683 mg, 923 μl, 5.29 mmol, 2.2 eq). The reaction mixture was stirred for 4 h. The reaction mixture was partitioned between EtOAc and water. The layers were separated. The aqueous layer was extracted with EtOAc. The combined organic layers were washed with brine, dried over anhydrous sodium sulfate, and concentrated in vacuo. The crude material was purified on silica column (heptane/EtOAc 0-60%) to afford the title compound (964 mg, 2.35 mmol, 98% yield) as a colourless oil. MS (ESI): 411.3 ([M+H]⁺).

### b) tert-butyl 8-[3-(4-benzyloxycarbonylpiperazine-1-carbonyl)-4-(trifluoromethyl)phenyl]-3,8-diazabicyclo[3.2.1]octane-3-carboxylate

A solution of benzyl 4-[5-fluoro-2-(trifluoromethyl)benzoyl]piperazine-1-carboxylate (911 mg, 2.22 mmol, 1.0 eq), tert-butyl 3,8-diazabicyclo[3.2.1]octane-3-carboxylate (566 mg, 2.66 mmol, 1.2 eq) and DIPEA (574 mg, 775 μL, 4.44 mmol, 2.0 eq) in DMSO (11.1 mL) was heated at 90° C. and stirred for 15 h. The reaction mixture was partitioned between EtOAc and water. The layers were separated. The aqueous layer was extracted with EtOAc. The combined organic layers were washed with brine, dried over anhydrous sodium sulfate, and concentrated in vacuo. The crude material was purified on silica column (heptane/EtOAc 0-70%) to afford the title compound (574 mg, 952 μmol, 43% yield) as a yellow solid. MS (ESI): 603.5 ([M+H]⁺).

### c) benzyl 4-[5-(3,8-diazabicyclo[3.2.1]octan-8-yl)-2-(trifluoromethyl)benzoyl]piperazine-1-carboxylate

To a stirred solution of tert-butyl 8-[3-(4-benzyloxycarbonylpiperazine-1-carbonyl)-4-(trifluoromethyl)phenyl]-3,8-diazabicyclo[3.2.1]octane-3-carboxylate (409 mg, 679 μmol, 1.0 eq) in DCM (5 mL) at room temperature was added TFA (1.55 g, 1.05 mL, 13.6 mmol, 20 eq). The reaction mixture was stirred for 20 h. The reaction mixture was partitioned between EtOAc and 2 M aqueous sodium hydroxide solution. The layers were separated. The aqueous layer was extracted with EtOAc. The combined organic layers were washed with brine, dried over anhydrous sodium sulfate, and concentrated in vacuo to afford the title compound (311 mg, 619 μmol, 91% yield) as alight brown solid. MS (ESI): 503.4 ([M+H]⁺).

### d) benzyl 4-[5-[3-(3-amino-6-chloro-pyridazin-4-yl)-3,8-diazabicyclo[3.2.1]octan-8-yl]-2-(trifluoromethyl)benzoyl]piperazine-1-carboxylate

To a suspension of benzyl 4-[5-(3,8-diazabicyclo[3.2.1]octan-8-yl)-2-(trifluoromethyl)benzoyl]piperazine-1-carboxylate (311 mg, 619 μmol, 1.0 eq) and 4-bromo-6-chloropyridazin-3-amine (168 mg, 805 μmol, 1.3 eq) in DMSO (1.38 mL) at room temperature was added K₂CO₃ (428 mg, 3.09 mmol, 5.0 eq). The reaction mixture was heated at 110° C. for 15 h. The reaction mixture was partitioned between EtOAc and water. The layers were separated. The aqueous layer was extracted with EtOAc. The combined organic layers were washed with brine, dried over anhydrous sodium sulfate, and concentrated in vacuo. The crude material was purified on silica column (heptane/EtOAc 0-100%) to afford the title compound (231 mg, 367 μmol, 59% yield) as a light brown solid. MS (ESI): 630.5 (³⁵Cl[M+H]⁺).

### e) benzyl 4-[5-[3-[3-amino-6-(2-hydroxyphenyl)pyridazin-4-yl]-3,8-diazabicyclo[3.2.1]octan-8-yl]-2-(trifluoromethyl)benzoyl]piperazine-1-carboxylate

To a stirred suspension of benzyl 4-[5-[3-(3-amino-6-chloro-pyridazin-4-yl)-3,8-diazabicyclo[3.2.1]octan-8-yl]-2-(trifluoromethyl)benzoyl]piperazine-1-carboxylate (231 mg, 367 μmol, 1.0 eq) and (2-hydroxyphenyl)boronic acid (126 mg, 917 μmol, 2.5 eq) in 1,4-dioxane (9 mL) and water (0.9 mL) at room temperature was added K₂CO₃ (177 mg, 1.28 mmol, 3.5 eq). The reaction mixture was degassed with argon for 10 min. RuPhos Pd G3 (30.7 mg, 36.7 μmol, 0.1 eq) was added. The reaction mixture was heated at 90° C. for 2 h. The reaction mixture was partitioned between EtOAc/THF and water. The layers were separated. The aqueous layer was extracted with EtOAc/THF. The combined organic layers were washed with brine, dried over anhydrous sodium sulfate, and concentrated in vacuo. The crude material was purified on silica column (DCM/MeOH 0-10%) to afford the title compound (67 mg, 97.4 μmol, 27% yield) as a light brown solid. MS (ESI): 688.7 ([M+H]⁺).

### f) [5-[3-[3-amino-6-(2-hydroxyphenyl)pyridazin-4-yl]-3,8-diazabicyclo[3.2.1]octan-8-yl]-2-(trifluoromethyl)phenyl]-piperazin-1-yl-methanone

A 25 ml two-necked round-bottomed flask was charged with benzyl 4-[5-[3-[3-amino-6-(2-hydroxyphenyl)pyridazin-4-yl]-3,8-diazabicyclo[3.2.1]octan-8-yl]-2-(trifluoromethyl)benzoyl]piperazine-1-carboxylate (67 mg, 97.4 μmol, 1.0 eq), MeOH (1 mL) and THF (1 mL). The flask was degassed with argon. After addition of the catalyst 10% Pd on charcoal (10.4 mg, 9.74 μmol, 0.1 eq), the flask was charged with hydrogen and stirred for 15 h under an atmosphere of of hydrogen gas. The catalyst was removed by filtration and washed with methanol. The filtrate was concentrated in vacuo to afford the title compound (48 mg, 86.7 μmol, 89% yield) as a light brown solid. MS (ESI): 554.5 ([M+H]⁺).

### g) rac-5-[4-[4-[5-[3-[3-amino-6-(2-hydroxyphenyl)pyridazin-4-yl]-3,8-diazabicyclo[3.2.1]octan-8-yl]-2-(trifluoromethyl)benzoyl]piperazine-1-carbonyl]-1-piperidyl]-2-(2,6-dioxo-3-piperidyl)isoindoline-1,3-dione

To a stirred solution of [5-[3-[3-amino-6-(2-hydroxyphenyl)pyridazin-4-yl]-3,8-diazabicyclo[3.2.1]octan-8-yl]-2-(trifluoromethyl)phenyl]-piperazin-1-yl-methanone (48 mg, 86.7 μmol, 1.0 eq), Ligase 15 (33.4 mg, 86.7 μmol, 1.0 eq) and HATU (72.5 mg, 191 μmol, 2.2 eq) in DMSO at room temperature was added DIPEA (33.6 mg, 45.4 μL, 260 μmol, 3.0 eq). The reaction mixture was stirred for 2 h. The purification was done by prep-HPLC to afford the title compound (36.9 mg, 35.7 μmol, 41% yield) as a yellow solid, 2,2,2-trifluoroacetate salt. MS (ESI): 921.4 ([M+H]⁺).

### Example 108

### N-[1-[3-amino-6-(2-hydroxyphenyl)pyridazin-4-yl]-4-phenyl-4-piperidyl]-2-[4-[4-[(2,6-dioxo-3-piperidyl)oxy]phenyl]-1-piperidyl]acetamide

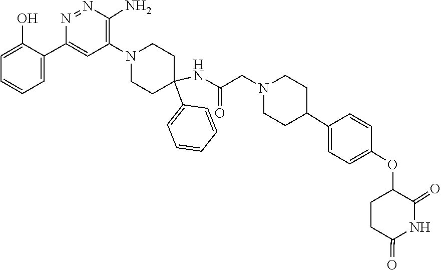 [SMILES: Nc1nnc(-c2ccccc2O)cc1N1CCC(NC(=O)CN2CCC(c3ccc(OC4CCC(=O)NC4=O)cc3)CC2)(c2ccccc2)CC1]

2-(6-amino-5-(4-amino-4-phenylpiperidin-1-yl)pyridazin-3-yl)phenol (40 mg, 111 μmol, 1.0 eq) was combined with Ligase 35 (46.6 mg, 122 μmol, 1.1 eq), HATU (84.2 mg, 221 μmol, 2.0 eq) and DIPEA (71.5 mg, 96.6 μL, 553 μmol, 5.0 eq) in DMF (400 μL). The brown solution was stirred at room temperature for 2 h and was then submitted to prep-HPLC for purification to afford the title compound (10 mg, 10.9 μmol, 9% yield) as a white solid, bis(2,2,2-trifluoroacetate). MS (ESI): 690.3401 ([M+H]⁺).

### Example 109

### 4-[3-[1-[9-[4-[4-[3-[3-amino-6-(2-hydroxyphenyl)pyridazin-4-yl]oxy-1-piperidyl]phenyl]piperazin-1-yl]nonyl]triazol-4-yl]propylamino]-2-(2,6-dioxo-3-piperidyl)isoindoline-1,3-dione

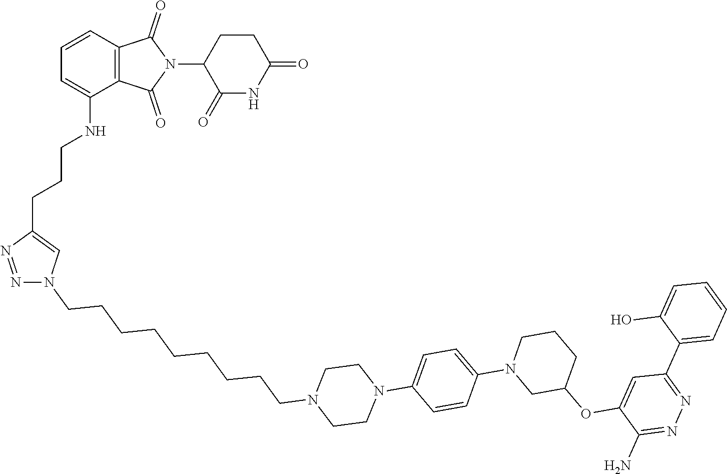 [SMILES: Nc1nnc(-c2ccccc2O)cc1OC1CCCN(c2ccc(N3CCN(CCCCCCCCCn4cc(CCCNc5cccc6c5C(=O)N(C5CCC(=O)NC5=O)C6=O)nn4)CC3)cc2)C1]

To a solution of 2-[6-amino-5-[[1-(4-piperazin-1-ylphenyl)-3-piperidyl]oxy]pyridazin-3-yl]phenol (40 mg, 89 μmol) and Ligase 57 (47 mg, 89 μmol) in THF (5 mL) was added dibutyltin dichloride (27 mg, 89 μmol, 19 μL) followed by the addition of trimethyl(phenyl)silane (12 mg, 107 μmol) and the reaction mixture was heated to 80° C. for 16 h. The reaction mixture was cooled to room temperature, diluted with water and extracted with EtOAc. The organic layer was washed with water, brine solution, dried over anhydrous sodium sulfate, filtered and concentrated under reduced pressure. The crude residue was purified by prep-HPLC to afford the title compound (9.37 mg, 9.8 μmol, 10% yield) as a light yellow solid. MS (ESI): 953.4 ([M+H]⁺).

### Example 110

### 4-[2-[1-[9-[4-[4-[3-[3-amino-6-(2-hydroxyphenyl)pyridazin-4-yl]oxy-1-piperidyl]phenyl]piperazin-1-yl]nonyl]triazol-4-yl]ethylamino]-2-(2,6-dioxo-3-piperidyl)isoindoline-1,3-dione

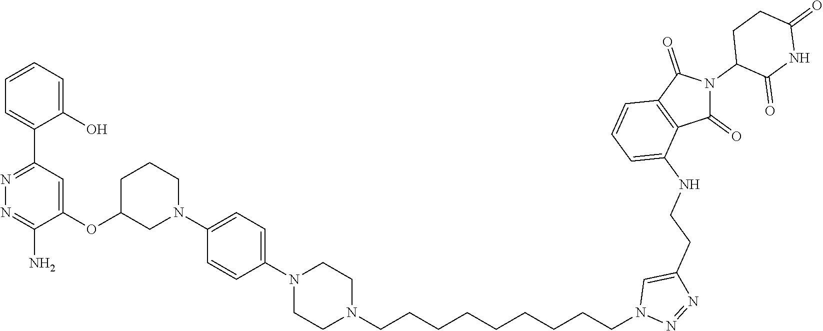 [SMILES: Nc1nnc(-c2ccccc2O)cc1OC1CCCN(c2ccc(N3CCN(CCCCCCCCCn4cc(CCNc5cccc6c5C(=O)N(C5CCC(=O)NC5=O)C6=O)nn4)CC3)cc2)C1]

To a solution of 2-[6-amino-5-[[1-(4-piperazin-1-ylphenyl)-3-piperidyl]oxy]pyridazin-3-yl]phenol (40 mg, 89 μmol) and Ligase 58 (45 mg, 89 μmol) in THF (5 mL) was added dibutyltin dichloride (27 mg, 89 μmol, 19 μL) followed by the addition of trimethyl(phenyl)silane (12 mg, 107 μmol) and the reaction mixture was heated to 80° C. for 16 h. The reaction mixture was cooled to room temperature, diluted with water and extracted with EtOAc. The organic layer was washed with water, brine solution, dried over anhydrous sodium sulfate, filtered and concentrated under reduced pressure. The crude residue was purified by prep-HPLC to afford the title compound (15.7 mg, 16.7 μmol, 18% yield) as a light yellow solid. MS (ESI): 939.4 ([M+H]⁺).

### Example 111

### 3-[7-[[1-[9-[4-[4-[3-[3-amino-6-(2-hydroxyphenyl)pyridazin-4-yl]oxy-1-piperidyl]phenyl]piperazin-1-yl]-9-oxo-nonyl]triazol-4-yl]methylamino]-1-oxo-isoindolin-2-yl]piperidine-2,6-dione

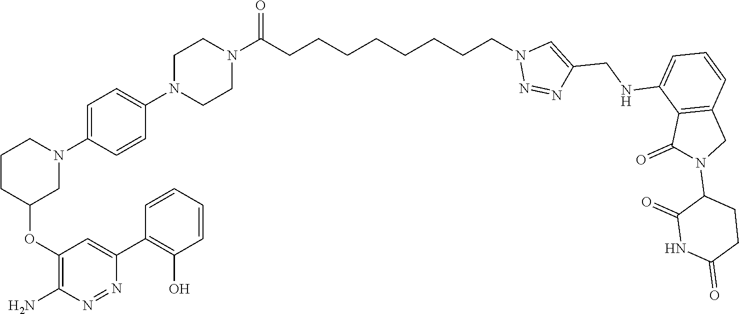 [SMILES: Nc1nnc(-c2ccccc2O)cc1OC1CCCN(c2ccc(N3CCN(C(=O)CCCCCCCCn4cc(CNc5cccc6c5C(=O)N(C5CCC(=O)NC5=O)C6)nn4)CC3)cc2)C1]

To a solution of Ligase 3 (14 mg, 22.9 μmol, TFA salt) and 2-[6-amino-5-[[1-(4-piperazin-1-ylphenyl)-3-piperidyl]oxy]pyridazin-3-yl]phenol (13 mg, 22.9 μmol, TFA) in dimethylformamide (0.4 mL) was added HATU (13 mg, 34.4 μmol) followed by DIPEA (9 mg, 68.9 μmol, 12 μL) and the reaction mixture was stirred at room temperature for 16 h. The reaction mixture was diluted with water and extracted with EtOAc. The volatiles were removed. The crude residue was purified by prep-HPLC to afford the title compound (2.65 mg, 2.66 μmol, 11% yield) as an off-white solid, trifluoroacetic acid salt. MS (ESI): 925.3 ([M+H]⁺).

### Example 112

### rac-3-[4-[1-[2-[4-[3-[1-[3-amino-6-(2-hydroxyphenyl)pyridazin-4-yl]azetidin-3-yl]oxyphenyl]piperazin-1-yl]-2-oxo-ethyl]-4-piperidyl]anilino]piperidine-2,6-dione

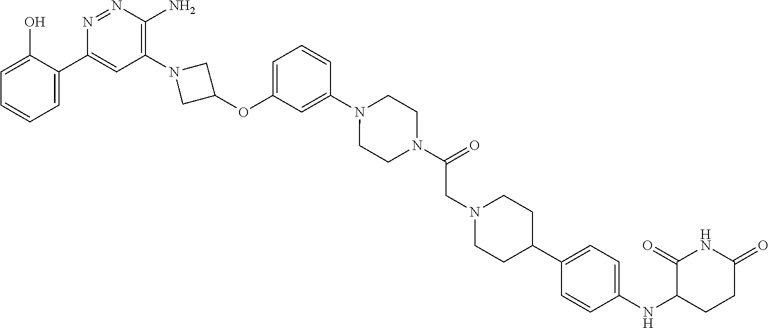 [SMILES: Nc1nnc(-c2ccccc2O)cc1N1CC(Oc2cccc(N3CCN(C(=O)CN4CCC(c5ccc(NC6CCC(=O)NC6=O)cc5)CC4)CC3)c2)C1]

To a stirred solution of 2-[6-amino-5-[3-(3-piperazin-1-ylphenoxy)azetidin-1-yl]pyridazin-3-yl]phenol (30 mg, 71.7 μmol, 1.0 eq), Ligase 51 (30.1 mg, 78.9 μmol, 1.1 eq) and DIPEA (46.3 mg, 62.6 μL, 358 μmol, 5.0 eq) in DMSO (300 μL) at room temperature was added HATU (54.5 mg, 143 μmol, 2.0 eq). The reaction mixture was stirred at room temperature for 1 h. The crude material was purified by prep-HPLC. The crude product was then dissolved in CH₂Cl₂, a few drops of Et₃N were added, and the mixture was concentrated in vacuo. The crude material was purified on silica column (DCM/MeOH 0-5%) to afford the title compound (11 mg, 9.44 μmol, 13% yield) as a white solid. MS (ESI): 746.4 ([M+H]⁺).

### Example 113

### rac-5-[4-[4-[3-[3-[3-amino-6-(2-hydroxyphenyl)pyridazin-4-yl]-3,8-diazabicyclo[3.2.1]octan-8-yl]phenoxy]piperidine-1-carbonyl]-1-piperidyl]-2-(2,6-dioxo-3-piperidyl)isoindoline-1,3-dione

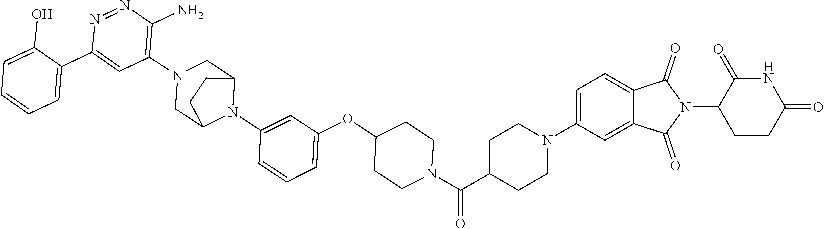 [SMILES: Nc1nnc(-c2ccccc2O)cc1N1CC2CCC(C1)N2c1cccc(OC2CCN(C(=O)C3CCN(c4ccc5c(c4)C(=O)N(C4CCC(=O)NC4=O)C5=O)CC3)CC2)c1]

### a) benzyl 4-(3-bromophenoxy)piperidine-1-carboxylate

To a stirred suspension of 4-(3-bromophenoxy)piperidine hydrochloride (2 g, 6.84 mmol, 1.0 eq) in DCM (60 mL) at room temperature was added Et₃N (2.08 g, 2.86 mL, 20.5 mmol, 3.0 eq). The reaction mixture was cooled to 0-5° C. and then benzyl chloroformate (1.28 g, 1.07 mL, 7.52 mmol, 1.1 eq) was added. The reaction mixture was stirred at room temperature for 3 h. The reaction mixture was concentrated in vacuo, and then taken up in EtOAc and water. The mixture was washed sequentially with 0.5 N aqueous NaOH, 0.5 N aqueous HCl, and brine. The organic layer was dried over Na₂SO₄ and concentrated in vacuo to afford the title compound (2.9 g, 6.84 mmol, 100% yield) as a colourless oil. MS (ESI): 392.1 ([M+H]⁺).

### b) tert-butyl 8-[3-[(1-benzyloxycarbonyl-4-piperidyl)oxy]phenyl]-3,8-diazabicyclo[3.2.1]octane-3-carboxylate

The reaction was carried out in sealed tube. To a stirred suspension of benzyl 4-(3-bromophenoxy)piperidine-1-carboxylate (2.9 g, 6.84 mmol, 1.0 eq) and tert-butyl (1R,5S)-3,8-diazabicyclo[3.2.1]octane-3-carboxylate (1.6 g, 7.52 mmol, 1.1 eq) in t-BuOH (22 mL) at room temperature was added K₂CO₃ (1.89 g, 13.7 mmol, 2.0 eq). The reaction mixture was degassed with argon for 10 min. RuPhos Pd G3 (572 mg, 684 μmol, 0.1 eq) was added. The reaction mixture was stirred at 115° C. for 16 h. The reaction mixture was cooled to room temperature, poured into EtOAc/THF (2:1). and washed sequentially with water and with brine. The organic layer was dried over Na₂SO₄ and concentrated in vacuo. The crude material was purified on silica column (heptane/EtOAc 0-50%) to afford the title compound (1.33 g, 2.42 mmol, 35% yield) as a colourless oil. MS (ESI): 522.5 ([M+H]⁺).

### c) benzyl 4-[3-(3,8-diazabicyclo[3.2.1]octan-8-yl)phenoxy]piperidine-1-carboxylate

To a stirred solution of tert-butyl 8-[3-[(1-benzyloxycarbonyl-4-piperidyl)oxy]phenyl]-3,8-diazabicyclo[3.2.1]octane-3-carboxylate (1.33 g, 2.55 mmol, 1.0 eq), in CH₂Cl₂ (10 mL) was added TFA (5.81 g, 3.93 mL, 51 mmol, 20 eq). The reaction mixture was stirred for 1 h. The reaction mixture was poured in EtOAc/THF (2:1) and washed with 0.5 N aqueous NaOH and with brine. The organic layer was dried over Na₂SO₄ and concentrated in vacuo to afford the title compound (1.22 g, 2.55 mmol, 100% yield) as a yellow oil. MS (ESI): 422.4 ([M+H]⁺).

### d) benzyl 4-[3-[3-(3-amino-6-chloro-pyridazin-4-yl)-3,8-diazabicyclo[3.2.1]octan-8-yl]phenoxy]piperidine-1-carboxylate

To a stirred solution of benzyl 4-[3-(3,8-diazabicyclo[3.2.1]octan-8-yl)phenoxy]piperidine-1-carboxylate (1.22 g, 2.55 mmol, 1.0 eq) and 4-bromo-6-chloropyridazin-3-amine (637 mg, 3.06 mmol, 1.2 eq) in DMSO (5 mL) at room temperature was added K₂CO₃ (1.76 g, 12.7 mmol, 5.0 eq). The reaction mixture was stirred at 110° C. for 3 h. The reaction mixture was poured in EtOAc/THF (2:1) and washed sequentially with water and with brine. The organic layer was dried over Na₂SO₄ and concentrated in vacuo. The crude material was purified on silica column (DCM/MeOH 0-5%) to afford the title compound (1.18 g, 2.04 mmol, 80% yield) as a light brown foam. MS (ESI): 549.5 (³⁵Cl[M+H]⁺).

### e) benzyl 4-[3-[3-[3-amino-6-(2-hydroxyphenyl)pyridazin-4-yl]-3,8-diazabicyclo[3.2.1]octan-8-yl]phenoxy]piperidine-1-carboxylate

To a stirred solution of benzyl 4-[3-[3-(3-amino-6-chloro-pyridazin-4-yl)-3,8-diazabicyclo[3.2.1]octan-8-yl]phenoxy]piperidine-1-carboxylate (0.98 g, 1.78 mmol, 1.0 eq) and (2-hydroxyphenyl)boronic acid (615 mg, 4.46 mmol, 2.5 eq) in dioxane (50 mL) and water (5 mL) at room temperature was added K₂CO₃ (863 mg, 6.25 mmol, 3.5 eq). The reaction mixture was degassed with argon for 10 min. RuPhos Pd G3 (149 mg, 178 μmol, 0.1 eq) was added. The reaction mixture was stirred at 90° C. for 1.5 h. The reaction mixture was poured into EtOAc/THF (1:1) and washed sequentially with water and with brine. The organic layer was dried over Na₂SO₄ and concentrated in vacuo. The crude material was purified on silica column (DCM/MeOH 0-5%) then on amino modified silica column (DCM/MeOH 0-5%) to afford the title compound (0.54 g, 801 μmol, 45% yield) as a yellow solid. MS (ESI): 607.5 ([M+H]⁺).

### f) 2-[6-amino-5-[8-[3-(4-piperidyloxy)phenyl]-3,8-diazabicyclo[3.2.1]octan-3-yl]pyridazin-3-yl]phenol

A solution of benzyl 4-[3-[3-[3-amino-6-(2-hydroxyphenyl)pyridazin-4-yl]-3,8-diazabicyclo[3.2.1]octan-8-yl]phenoxy]piperidine-1-carboxylate (0.54 g, 890 μmol, 1.0 eq) in methanol (120 mL) and THF (40 mL) at room temperature was degassed with argon for 5 min. 10% Pd on charcoal (189 mg, 178 μmol, 0.2 eq) was added. The reaction mixture was degassed with H₂ for 10 min. The reaction mixture was then stirred under an H₂ balloon at room temperature for 1.5 h. The reaction mixture was filtered through a Sartorius funnel, then concentrated in vacuo to afford the title compound (453 mg, 863 μmol, 97% yield) as a yellow solid. MS (ESI): 473.5 ([M+H]⁺).

### g) rac-5-[4-[4-[3-[3-[3-amino-6-(2-hydroxyphenyl)pyridazin-4-yl]-3,8-diazabicyclo[3.2.1]octan-8-yl]phenoxy]piperidine-1-carbonyl]-1-piperidyl]-2-(2,6-dioxo-3-piperidyl)isoindoline-1,3-dione

To a stirred solution of 2-[6-amino-5-[8-[3-(4-piperidyloxy)phenyl]-3,8-diazabicyclo[3.2.1]octan-3-yl]pyridazin-3-yl]phenol (60 mg, 127 μmol, 1.0 eq), Ligase 15 (48.9 mg, 127 μmol, 1.0 eq) and DIPEA (65.6 mg, 88.7 μL, 508 μmol, 4.0 eq) in DMSO (1 mL) at room temperature was added HATU (96.5 mg, 254 μmol, 2.0 eq). The reaction mixture was stirred at room temperature for 2 h. The crude material was purified by prep-HPLC to afford the title compound (37 mg, 32.9 μmol, 26% yield) as a yellow solid, bis-(2,2,2-trifluoroacetic acid)salt. MS (ESI): 840.4 ([M+H]⁺).

### Example 114

### rac-3-[4-[1-[2-[4-[3-[3-[3-amino-6-(2-hydroxyphenyl)pyridazin-4-yl]-3,8-diazabicyclo[3.2.1]octan-8-yl]phenoxy]-1-piperidyl]-2-oxo-ethyl]-4-piperidyl]anilino]piperidine-2,6-dione

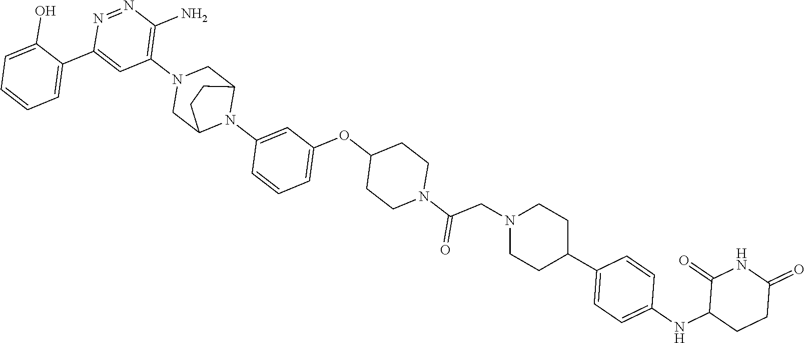 [SMILES: Nc1nnc(-c2ccccc2O)cc1N1CC2CCC(C1)N2c1cccc(OC2CCN(C(=O)CN3CCC(c4ccc(NC5CCC(=O)NC5=O)cc4)CC3)CC2)c1]

To a stirred solution of 2-[6-amino-5-[8-[3-(4-piperidyloxy)phenyl]-3,8-diazabicyclo[3.2.1]octan-3-yl]pyridazin-3-yl]phenol (60 mg, 127 μmol, 1.0 eq), Ligase 51 (48.5 mg, 127 μmol, 1.0 eq) and DIPEA (82 mg, 111 μL, 635 μmol, 5.0 eq) in DMSO (1 mL) at room temperature was added HATU (96.5 mg, 254 μmol, 2.0 eq). The reaction mixture was stirred at room temperature for 2 h. The crude material was purified by prep-HPLC. The product was lyophilised to afford the title compound (40 mg, 35 μmol, 28% yield) as a yellow solid, bis-(2,2,2-trifluoroacetic acid)salt. MS (ESI): 800.4 ([M+H]⁺).

### Example 115

### N-(1-(3-amino-6-(2-hydroxyphenyl)pyridazin-4-yl)-4-phenylpiperidin-4-yl)-3-(4-(4-((2,6-dioxopiperidin-3-yl)oxy)phenyl)piperidin-1-yl)propanamide diformate

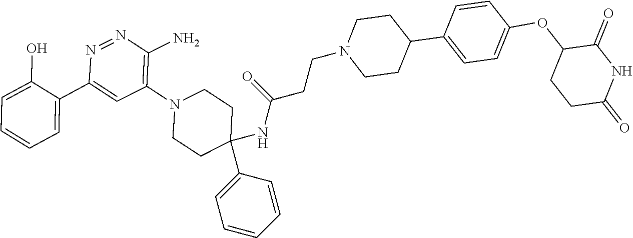 [SMILES: Nc1nnc(-c2ccccc2O)cc1N1CCC(NC(=O)CCN2CCC(c3ccc(OC4CCC(=O)NC4=O)cc3)CC2)(c2ccccc2)CC1]

2-(6-amino-5-(4-amino-4-phenylpiperidin-1-yl)pyridazin-3-yl)phenol (20 mg, 55.3 μmol, 1.0 eq) was combined with Ligase 40 (19.9 mg, 55.3 μmol, 1.0 eq), HATU (42.1 mg, 111 μmol, 2.0 eq) and DIPEA (21.5 mg, 29 μL, 166 μmol, 3.0 eq) in DMF (200 μL). The brown solution was stirred at room temperature for 2 h. The reaction was purified by prep-HPLC to afford the title compound (5.4 mg, 5.25 μmol, 9% yield) as a white solid. MS (ESI): 704.3555 ([M+H]⁺).

### Example 116

### rac-5-[4-[4-[3-[3-[3-amino-6-(2-hydroxyphenyl)pyridazin-4-yl]-3,8-diazabicyclo[3.2.1]octan-8-yl]-2-methyl-benzoyl]piperazine-1-carbonyl]-1-piperidyl]-2-(2,6-dioxo-3-piperidyl)isoindoline-1,3-dione

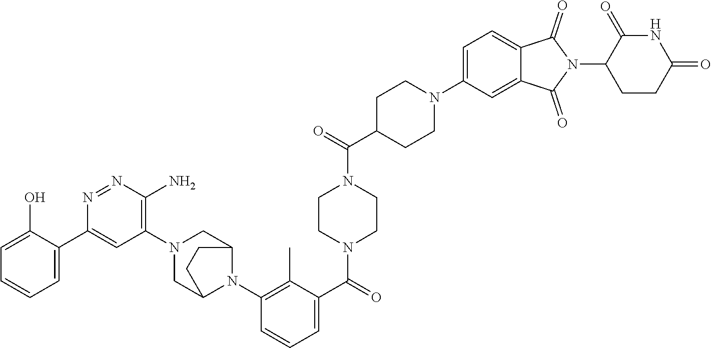 [SMILES: Cc1c(C(=O)N2CCN(C(=O)C3CCN(c4ccc5c(c4)C(=O)N(C4CCC(=O)NC4=O)C5=O)CC3)CC2)cccc1N1C2CCC1CN(c1cc(-c3ccccc3O)nnc1N)C2]

### a) benzyl 4-(3-bromo-2-methyl-benzoyl)piperazine-1-carboxylate

To a stirred solution of 3-bromo-2-methylbenzoic acid (1.5 g, 6.98 mmol, 1.0 eq), benzyl piperazine-1-carboxylate (2 g, 1.75 mL, 9.07 mmol, 1.3 eq) and HATU (5.57 g, 14.6 mmol, 2.1 eq) in DMSO (5.63 mL) at room temperature was added DIPEA (1.98 g, 2.68 mL, 15.3 mmol, 2.2 eq). The reaction mixture was stirred for 15 h. The reaction mixture was partitioned between EtOAc and water. The layers were separated. The aqueous layer was extracted with EtOAc. The combined organic layers were washed with brine, dried over anhydrous sodium sulfate, and concentrated in vacuo. The crude material was purified on silica column (heptane/EtOAc 0-50%) to afford the title compound (3.016 g, 7.23 mmol, 100% yield) as a colourless oil. MS (ESI): 419.1 ([M+H]⁺).

### b) tert-butyl 8-[3-(4-benzyloxycarbonylpiperazine-1-carbonyl)-2-methyl-phenyl]-3,8-diazabicyclo[3.2.1]octane-3-carboxylate

To a stirred suspension of benzyl 4-(3-bromo-2-methyl-benzoyl)piperazine-1-carboxylate (1 g, 2.4 mmol, 1.0 eq) and tert-butyl 3,8-diazabicyclo[3.2.1]octane-3-carboxylate (534 mg, 2.52 mmol, 1.05 eq) in t-BuOH (12.3 mL) at room temperature was added K₂CO₃ (662 mg, 4.79 mmol, 2.0 eq). The reaction was degassed with argon for 5 min. RuPhos Pd G3 (200 mg, 240 μmol, 0.1 eq) was added. The reaction mixture was stirred at reflux for 2 days. The reaction mixture was transferred to a vial and the vial was sealed with a cap. The reaction mixture was heated to 120° C. and stirred for 2 days. The reaction mixture was poured into EtOAc and washed sequentially with water and with brine. The organic layer was dried over Na₂SO₄ and concentrated in vacuo. The crude material was purified on silica column (heptane/EtOAc 0-50%) to afford the title compound (378 mg, 517 μmol, 22% yield) as a yellow oil. MS (ESI): 549 ([M+H]⁺).

### c) benzyl 4-[3-(3,8-diazabicyclo[3.2.1]octan-8-yl)-2-methyl-benzoyl]piperazine-1-carboxylate

To a stirred solution of tert-butyl 8-[3-(4-benzyloxycarbonylpiperazine-1-carbonyl)-2-methyl-phenyl]-3,8-diazabicyclo[3.2.1]octane-3-carboxylate (378 mg, 689 μmol, 1.0 eq) in DCM (5 mL) at room temperature was added trifluoroacetic acid (1.48 g, 1 mL, 13 mmol, 18.8 eq). The reaction mixture was stirred for 3 d. The reaction mixture was partitioned between EtOAc/THF (1:1) and 2 M aqueous sodium hydroxide solution. The layers were separated. The aqueous layer was extracted with EtOAc. The combined organic layers were washed with brine, dried over anhydrous sodium sulfate, and concentrated in vacuo to afford the title compound (278 mg, 620 μmol, 90% yield) as an off-white solid. MS (ESI): 449.5 ([M+H]⁺).

### d) benzyl 4-[3-[3-(3-amino-6-chloro-pyridazin-4-yl)-3,8-diazabicyclo[3.2.1]octan-8-yl]-2-methyl-benzoyl]piperazine-1-carboxylate

To a stirred suspension of benzyl 4-[3-(3,8-diazabicyclo[3.2.1]octan-8-yl)-2-methyl-benzoyl]piperazine-1-carboxylate (278 mg, 620 μmol, 1.0 eq) and 4-bromo-6-chloropyridazin-3-amine (168 mg, 806 μmol, 1.3 eq) in DMSO (1.38 mL) at room temperature was added K₂CO₃ (428 mg, 3.1 mmol, 5.0 eq). The reaction mixture was heated at 110° C. for 15 h. The reaction mixture was partitioned between EtOAc and water. The layers were separated. The aqueous layer was extracted with EtOAc. The combined organic layers were washed with brine, dried over anhydrous sodium sulfate, and concentrated in vacuo. The crude material was purified on silica column (DCM/MeOH 0-10%) to afford the title compound (149.5 mg, 260 μmol, 42% yield) as a light brown solid. MS (ESI): 576.5 (³⁵Cl[M+H]⁺).

### e) benzyl 4-[3-[3-[3-amino-6-(2-hydroxyphenyl)pyridazin-4-yl]-3,8-diazabicyclo[3.2.1]octan-8-yl]-2-methyl-benzoyl]piperazine-1-carboxylate and [3-[3-[3-amino-6-(2-hydroxyphenyl)pyridazin-4-yl]-3,8-diazabicyclo[3.2.1]octan-8-yl]-2-methyl-phenyl]-piperazin-1-yl-methanone

To a stirred suspension of benzyl 4-[3-[3-(3-amino-6-chloro-pyridazin-4-yl)-3,8-diazabicyclo[3.2.1]octan-8-yl]-2-methyl-benzoyl]piperazine-1-carboxylate (149.5 mg, 260 μmol, 1.0 eq) and (2-hydroxyphenyl)boronic acid (89.5 mg, 649 μmol, 2.5 eq) in 1,4-dioxane (5.82 mL) and water (0.9 mL) at room temperature was added K₂CO₃ (126 mg, 908 μmol, 3.5 eq). The reaction mixture was degassed with argon for 10 min. RuPhos Pd G3 (21.7 mg, 26 μmol, 0.1 eq) was added. The reaction mixture was heated at 90° C. for 2 h. The reaction mixture was partitioned between EtOAc/THF and water. The layers were separated. The aqueous layer was extracted with EtOAc/THF. The combined organic layers were washed with brine, dried over anhydrous sodium sulfate, and concentrated in vacuo. The crude material was purified on silica column (DCM/MeOH 0-10%) to afford the title compound 1 (23.5 mg, 37.1 μmol, 14% yield) as a light brown solid. MS (ESI): 634.6 ([M+H]⁺), and the title compound 2 (71 mg, 142 μmol, 55% yield) as a light brown solid. MS (ESI): 500.4 ([M+H]⁺).

### f) rac-5-[4-[4-[3-[3-[3-amino-6-(2-hydroxyphenyl)pyridazin-4-yl]-3,8-diazabicyclo[3.2.1]octan-8-yl]-2-methyl-benzoyl]piperazine-1-carbonyl]-1-piperidyl]-2-(2,6-dioxo-3-piperidyl)isoindoline-1,3-dione

To a stirred solution of [3-[3-[3-amino-6-(2-hydroxyphenyl)pyridazin-4-yl]-3,8-diazabicyclo[3.2.1]octan-8-yl]-2-methyl-phenyl]-piperazin-1-yl-methanone (30 mg, 60 μmol, 1.0 eq), Ligase 15 (23.1 mg, 60 μmol, 1.0 eq) and HATU (50.2 mg, 132 μmol, 2.2 eq) in DMSO at room temperature was added DIPEA (23.3 mg, 31.5 μL, 180 μmol, 3.0 eq). The reaction mixture was stirred for 2 h. The purification was done by prep-HPLC to afford the title compound (9.9 mg, 8.86 μmol, 15% yield) as a yellow solid, bis-(2,2,2-trifluoroacetic acid)salt. MS (ESI): 867.6 ([M+H]⁺).

### Example 117

### rac-5-[4-[3-[4-[3-[3-[3-amino-6-(2-hydroxyphenyl)pyridazin-4-yl]-3,8-diazabicyclo[3.2.1]octan-8-yl]-2-methyl-benzoyl]piperazin-1-yl]-3-oxo-propyl]-1-piperidyl]-2-(2,6-dioxo-3-piperidyl)isoindoline-1,3-dione

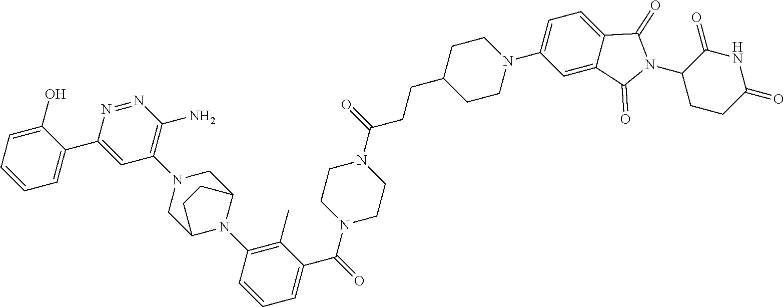 [SMILES: Cc1c(C(=O)N2CCN(C(=O)CCC3CCN(c4ccc5c(c4)C(=O)N(C4CCC(=O)NC4=O)C5=O)CC3)CC2)cccc1N1C2CCC1CN(c1cc(-c3ccccc3O)nnc1N)C2]

To a solution of [3-[3-[3-amino-6-(2-hydroxyphenyl)pyridazin-4-yl]-3,8-diazabicyclo[3.2.1]octan-8-yl]-2-methyl-phenyl]-piperazin-1-yl-methanone (30 mg, 60 μmol, 1.0 eq), Ligase 12 acid (24.8 mg, 60 μmol, 1.0 eq) and HATU (50.2 mg, 132 μmol, 2.2 eq) in DMSO at room temperature was added DIPEA (23.3 mg, 31.5 μL, 180 μmol, 3.0 eq). The reaction mixture was stirred for 2 h. The purification was done by prep-HPLC to afford the title compound (29 mg, 23.8 μmol, 40% yield) as a yellow solid, bis-(2,2,2-trifluoroacetic acid)salt. MS (ESI): 895.6 ([M+H]⁺).

### Example 118

### rac-1-[[3-[3-[3-amino-6-(2-hydroxyphenyl)pyridazin-4-yl]-3,8-diazabicyclo[3.2.1]octan-8-yl]phenyl]methyl]-N-[1-[2-(2,6-dioxo-3-piperidyl)-1,3-dioxo-isoindolin-5-yl]-4-piperidyl]-N-methyl-piperidine-4-carboxamide

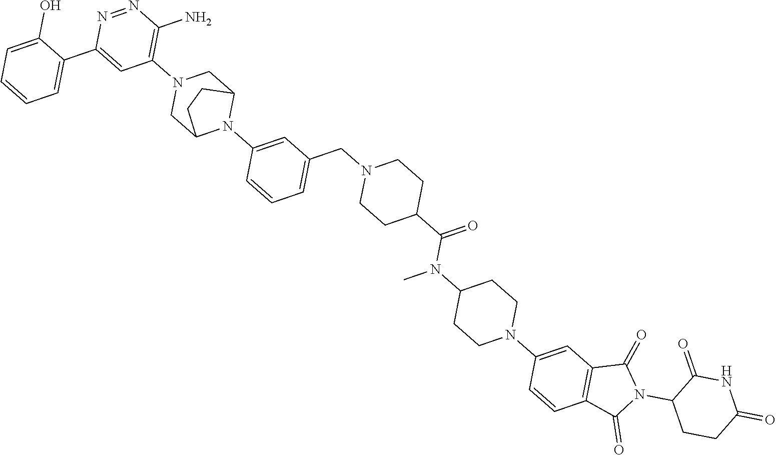 [SMILES: CN(C(=O)C1CCN(Cc2cccc(N3C4CCC3CN(c3cc(-c5ccccc5O)nnc3N)C4)c2)CC1)C1CCN(c2ccc3c(c2)C(=O)N(C2CCC(=O)NC2=O)C3=O)CC1]

To a stirred solution of 1-[[3-[3-[3-amino-6-(2-hydroxyphenyl)pyridazin-4-yl]-3,8-diazabicyclo[3.2.1]octan-8-yl]phenyl]methyl]piperidine-4-carboxylic acid (60 mg, 117 μmol, 1.0 eq), Ligase 56 (43.2 mg, 117 μmol, 1.0 eq) and DIPEA (75.3 mg, 102 μL, 583 μmol, 5.0 eq) in DMSO (0.6 mL) at room temperature was added HATU (88.7 mg, 233 μmol, 2.0 eq). The reaction mixture was stirred at room temperature for 3 h. The reaction mixture was poured into THF/EtOAc (2:1) and washed sequentially with water and brine. The crude material was purified by prep-HPLC to afford the title compound (20 mg, 18.3 μmol, 16% yield) as a yellow solid, bis-(2,2,2-trifluoroacetic acid)salt. MS (ESI): 867.4 ([M+H]⁺).

### Example 119

### N-(1-(1-(3-amino-6-(2-hydroxyphenyl)pyridazin-4-yl)-4-phenylpiperidine-4-carbonyl)piperidin-4-yl)-1-(2-(2,6-dioxopiperidin-3-yl)-1,3-dioxoisoindolin-5-yl)piperidine-4-carboxamide

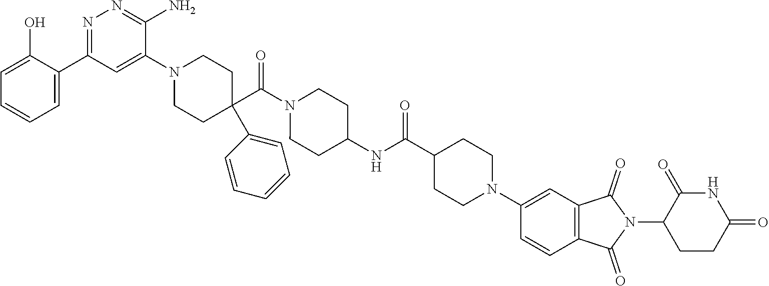 [SMILES: Nc1nnc(-c2ccccc2O)cc1N1CCC(C(=O)N2CCC(NC(=O)C3CCN(c4ccc5c(c4)C(=O)N(C4CCC(=O)NC4=O)C5=O)CC3)CC2)(c2ccccc2)CC1]

### a) tert-butyl N-[1-[1-(3-amino-6-chloro-pyridazin-4-yl)-4-phenyl-piperidine-4-carbonyl]-4-piperidyl]carbamate

To a solution of potassium 4-(3-amino-6-chloro-pyridazin-4-yl)-1-phenyl-piperazine-2-carboxylate (59 mg, 158 μmol, 1.0 eq), HATU (56.8 mg, 149 μmol, 1.2 eq) and DIPEA (161 mg, 218 μL, 1.25 mmol, 10.0 eq) in DMF (2 mL) was added tert-butyl 4-(aminomethyl)piperidine-1-carboxylate (385 mg, 1.8 mmol, 1.5 eq). The reaction mixture was stirred at room temperature for 1 h. The reaction mixture was concentrated in vacuo. The crude material was purified on silica column (heptane/EtOAc 0-100%) to afford the title compound (65 mg, 126 μmol, 80% yield) as a brown solid. MS (ESI): 515.4 ([M+H]⁺).

### b) tert-butyl N-[1-[1-[3-amino-6-(2-hydroxyphenyl)pyridazin-4-yl]-4-phenyl-piperidine-4-carbonyl]-4-piperidyl]carbamate

A suspension of tert-butyl (1-(1-(3-amino-6-chloropyridazin-4-yl)-4-phenylpiperidine-4-carbonyl)piperidin-4-yl)carbamate (65 mg, 126 μmol, 1.0 eq), (2-hydroxyphenyl)boronic acid (43.5 mg, 316 μmol, 2.5 eq), potassium carbonate (52.3 mg, 379 μmol, 3.0 eq) and RuPhos Pd G3 (5.28 mg, 6.31 μmol, 0.05 eq) in a mixture of degassed dioxane (3 mL) and water (0.3 mL) was stirred at 110° C. for 2 h under argon. The reaction mixture was poured in saturated NaHCO₃ and extracted with EtOAc. The organic layers were combined, washed with water and brine. The organic layers were dried over Na₂SO₄ and concentrated in vacuo. The crude material was purified on silica column (heptane/EtOAc 0-100%) to afford the title compound (45 mg, 79 μmol, 62% yield) as a brown foam. MS (ESI): 573.5 ([M+H]⁺).

### c) [1-[3-amino-6-(2-hydroxyphenyl)pyridazin-4-yl]-4-phenyl-4-piperidyl]-(4-amino-1-piperidyl)methanone

To a cooled (0° C.) solution of tert-butyl (1-(1-(3-amino-6-(2-hydroxyphenyl)pyridazin-4-yl)-4-phenylpiperidine-4-carbonyl)piperidin-4-yl)carbamate (40 mg, 70 μmol, 1.0 eq) in DCM (2 mL) was added 4M HCl in dioxane (52.4 μL, 210 μmol, 3.0 eq). The reaction mixture was allowed to reach room temperature and stirred for 16 h. The reaction mixture was filtered and the solid was dried in vacuo to afford the title compound (22 mg, 4 μmol, 62%) as a white solid, hydrochloride salt. MS (ESI): 473.5 ([M+H]⁺).

### d) N-(1-(1-(3-amino-6-(2-hydroxyphenyl)pyridazin-4-yl)-4-phenylpiperidine-4-carbonyl)piperidin-4-yl)-1-(2-(2,6-dioxopiperidin-3-yl)-1,3-dioxoisoindolin-5-yl)piperidine-4-carboxamide

Ligase 15 (10.9 mg, 28.3 μmol, 1.2 eq) was dissolved in dry DMF (400 μL). DIPEA (7.62 mg, 10.3 μL, 58.9 μmol, 2.5 eq) and HATU (9.86 mg, 25.9 μmol, 1.1 eq) were added and the mixture was stirred at room temperature for 10 min. (1-(3-amino-6-(2-hydroxyphenyl)pyridazin-4-yl)-4-phenylpiperidin-4-yl)(4-aminopiperidin-1-yl)methanone, hydrochloride salt (12 mg, 23.6 μmol, 1.0 eq) was added and the mixture was stirred at room temperature for 2 h. The reaction mixture was purified directly by prep-HPLC to afford the title compound (17.4 mg, 18.2 μmol, 77% yield) as a yellow solid, 2,2,2-trifluoroacetate salt. MS (ESI): 840.6 ([M+H]⁺).

### Example 120

### 5-(4-(4-(1-(3-amino-6-(2-hydroxyphenyl)pyridazin-4-yl)-4-phenylpiperidine-4-carbonyl)piperazine-1-carbonyl)piperidin-1-yl)-2-(2,6-dioxopiperidin-3-yl)isoindoline-1,3-dione

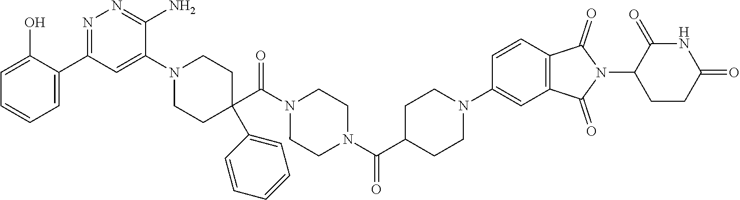 [SMILES: Nc1nnc(-c2ccccc2O)cc1N1CCC(C(=O)N2CCN(C(=O)C3CCN(c4ccc5c(c4)C(=O)N(C4CCC(=O)NC4=O)C5=O)CC3)CC2)(c2ccccc2)CC1]

### a) tert-butyl 4-[1-(3-amino-6-chloro-pyridazin-4-yl)-4-phenyl-piperidine-4-carbonyl]piperazine-1-carboxylate

To a solution of potassium 4-(3-amino-6-chloro-pyridazin-4-yl)-1-phenyl-piperazine-2-carboxylate (46 mg, 125 μmol, 1.0 eq), HATU (56.8 mg, 149 μmol, 1.2 eq) and DIPEA (161 mg, 218 μL, 1.25 mmol, 10.0 eq) in DMF (2 mL) was added tert-butyl piperazine-1-carboxylate (34.8 mg, 187 μmol, 1.5 eq). The reaction mixture was stirred at room temperature for 16 h. The reaction mixture was concentrated in vacuo. The crude material was purified on silica column (heptane/EtOAc 0-100%) to afford the title compound (60 mg, 120 μmol, 96% yield) as a light brown solid. MS (ESI): 501.4 ([M+H]⁺).

### b) tert-butyl 4-[1-[3-amino-6-(2-hydroxyphenyl)pyridazin-4-yl]-4-phenyl-piperidine-4-carbonyl]piperazine-1-carboxylate

A suspension of tert-butyl 4-(1-(3-amino-6-chloropyridazin-4-yl)-4-phenylpiperidine-4-carbonyl)piperazine-1-carboxylate (60 mg, 120 μmol, 1.0 eq), (2-hydroxyphenyl)boronic acid (41.3 mg, 299 μmol, 2.5 eq), potassium carbonate (49.7 mg, 359 μmol, 3.0 eq) and RuPhos Pd G3 (5.01 mg, 5.99 μmol, 0.05 eq) in a mixture of degassed dioxane (3 mL) and water (0.3 mL) was stirred at 110° C. for 2 h under argon. The reaction mixture was poured in saturated NaHCO₃ and extracted with EtOAc. The organic layers were combined, washed with water and brine. The organic layers were dried over Na₂SO₄ and concentrated in vacuo. The crude material was purified on silica column (heptane/EtOAc 0-100%) to afford the title compound (25 mg, 45 μmol, 37% yield) as a light brown solid. MS (ESI): 559.4 ([M+H]⁺).

### c) [1-[3-amino-6-(2-hydroxyphenyl)pyridazin-4-yl]-4-phenyl-4-piperidyl]-piperazin-1-yl-methanone

To a cooled (0° C.) solution of tert-butyl 4-(1-(3-amino-6-(2-hydroxyphenyl)pyridazin-4-yl)-4-phenylpiperidine-4-carbonyl)piperazine-1-carboxylate (20 mg, 35.8 μmol, 1.0 eq) in DCM (1 mL) was added 4M HCl in dioxane (26.8 μL, 107 μmol, 3.0 eq). The reaction mixture was allowed to reach room temperature and stirred for 16 h. The reaction mixture was filtered and the solid was dried in vacuo to afford the title compound (12 mg, 24 μmol, 68% yield) as a white hydrochloride salt. MS (ESI): 459.4 ([M+H]⁺).

### d) 5-(4-(4-(1-(3-amino-6-(2-hydroxyphenyl)pyridazin-4-yl)-4-phenylpiperidine-4-carbonyl)piperazine-1-carbonyl)piperidin-1-yl)-2-(2,6-dioxopiperidin-3-yl)isoindoline-1,3-dione

Ligase 15 (5.61 mg, 14.5 μmol, 1.2 eq) was dissolved in dry DMF (400 μL). DIPEA (3.92 mg, 5.29 μL, 30.3 μmol, 2.5 eq) and HATU (5.07 mg, 13.3 μmol, 1.1 eq) were added and the mixture was stirred at room temperature for 10 min. (1-(3-amino-6-(2-hydroxyphenyl)pyridazin-4-yl)-4-phenylpiperidin-4-yl)(piperazin-1-yl)methanone hydrochloride (6 mg, 12.1 μmol, 1.0 eq) was added and the mixture was stirred at room temperature for 2 h. The reaction mixture was purified directly by prep-HPLC to afford the title compound (3.6 mg, 3.83 μmol, 31% yield) as a yellow solid, 2,2,2-trifluoroacetate salt. MS (ESI): 826.6 ([M+H]⁺).

### Example 121

### 5-(4-(6-(1-(3-amino-6-(2-hydroxyphenyl)pyridazin-4-yl)-4-phenylpiperidine-4-carbonyl)-2,6-diazaspiro[3.3]heptane-2-carbonyl)piperidin-1-yl)-2-(2,6-dioxopiperidin-3-yl)isoindoline-1,3-dione

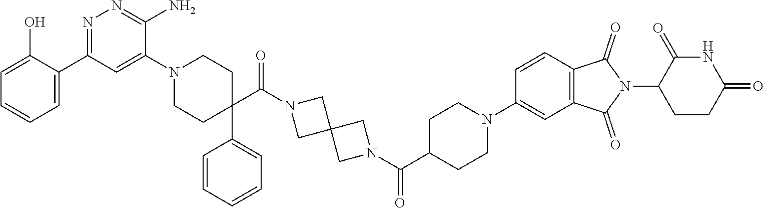 [SMILES: Nc1nnc(-c2ccccc2O)cc1N1CCC(C(=O)N2CC3(CN(C(=O)C4CCN(c5ccc6c(c5)C(=O)N(C5CCC(=O)NC5=O)C6=O)CC4)C3)C2)(c2ccccc2)CC1]

### a) tert-butyl 6-[1-(3-amino-6-chloro-pyridazin-4-yl)-4-phenyl-piperidine-4-carbonyl]-2,6-diazaspiro[3.3]heptane-2-carboxylate

To a solution of potassium 4-(3-amino-6-chloro-pyridazin-4-yl)-1-phenyl-piperazine-2-carboxylate (445 mg, 1.2 mmol, 1.0 eq), HATU (548 mg, 1.44 mmol, 1.2 eq) and DIPEA (932 mg, 1.26 mL, 7.21 mmol, 6.0 eq) in DMF (5 mL) was added tert-butyl piperazine-1-carboxylate (34.8 mg, 187 μmol, 1.5 eq). The reaction mixture was stirred at room temperature for 16 h. The reaction mixture was concentrated in vacuo. The crude material was purified on silica column (heptane/EtOAc 0-100%) to afford the title compound (643 mg, 1.32 mmol, 1.1 eq) as a light brown solid. MS (ESI): 513.4 ([M+H]⁺).

### b) tert-butyl 6-[1-[3-amino-6-(2-hydroxyphenyl)pyridazin-4-yl]-4-phenyl-piperidine-4-carbonyl]-2,6-diazaspiro[3.3]heptane-2-carboxylate

A suspension of tert-butyl 6-(1-(3-amino-6-chloropyridazin-4-yl)-4-phenylpiperidine-4-carbonyl)-2,6-diazaspiro[3.3]heptane-2-carboxylate (600 mg, 1.17 mmol, 1.0 eq), (2-hydroxyphenyl)boronic acid (403 mg, 2.92 mmol, 2.5 eq), potassium carbonate (485 mg, 3.51 mmol, 3.0 eq) and RuPhos Pd G3 (48.9 mg, 58.5 μmol, 0.05 eq) in a mixture of degassed dioxane (3 mL) and water (0.3 mL) was stirred at 110° C. for 2 h under argon. The reaction mixture was poured in saturated NaHCO₃ and extracted with EtOAc. The organic layers were combined, washed with water and brine. The organic layers were dried over Na₂SO₄ and concentrated in vacuo. The crude material was purified on silica column (DCM/MeOH 0-10%) to afford the title compound (225 mg, 395 μmol, 33% yield) as a light brown solid. MS (ESI): 571.4 ([M+H]⁺).

### c) [1-[3-amino-6-(2-hydroxyphenyl)pyridazin-4-yl]-4-phenyl-4-piperidyl]-(2,6-diazaspiro[3.3]heptan-2-yl)methanone

A solution of tert-butyl 6-(1-(3-amino-6-(2-hydroxyphenyl)pyridazin-4-yl)-4-phenylpiperidine-4-carbonyl)-2,6-diazaspiro[3.3]heptane-2-carboxylate (225 mg, 394 μmol, 1.0 eq) in 1,1,1,3,3,3-hexafluoro-2-propanol (3.31 g, 2.07 mL, 19.7 mmol, 50.0 eq) in a sealed tube was stirred at 120° C. for 48 h. The reaction mixture was cooled to room temperature and concentrated in vacuo to afford the title compound (110 mg, 234 μmol, 59% yield) as a light brown solid. MS (ESI): 471.4 ([M+H]⁺).

### d) 5-(4-(6-(1-(3-amino-6-(2-hydroxyphenyl)pyridazin-4-yl)-4-phenylpiperidine-4-carbonyl)-2,6-diazaspiro[3.3]heptane-2-carbonyl)piperidin-1-yl)-2-(2,6-dioxopiperidin-3-yl)isoindoline-1,3-dione

Ligase 15 (16.7 mg, 43.4 μmol, 1.2 eq) was dissolved in dry DMF (400 μL). DIPEA (11.7 mg, 15.8 μL, 90.3 μmol, 2.5 eq) and HATU (17.9 mg, 47 μmol, 1.3 eq) were added and the mixture was stirred at room temperature for 10 min. (1-(3-amino-6-(2-hydroxyphenyl)pyridazin-4-yl)-4-phenylpiperidin-4-yl)(2,6-diazaspiro[3.3]heptan-2-yl)methanone (17 mg, 36.1 μmol, 1.0 eq) was added and the mixture was stirred at room temperature for 2 h. The reaction mixture was purified directly by prep-HPLC to afford the title compound (21.6 mg, 24.4 μmol, 67.6% yield) as a yellow solid, formate salt. MS (ESI): 838.6 ([M+H]⁺).

### Example 122

### 5-(4-(2-(6-(1-(3-amino-6-(2-hydroxyphenyl)pyridazin-4-yl)-4-phenylpiperidine-4-carbonyl)-2,6-diazaspiro[3.3]heptan-2-yl)-2-oxoethyl)piperidin-1-yl)-2-(2,6-dioxopiperidin-3-yl)isoindoline-1,3-dione

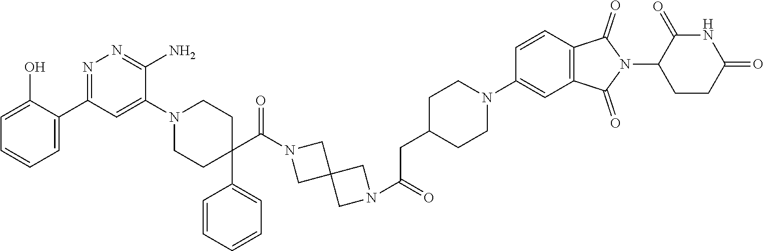 [SMILES: Nc1nnc(-c2ccccc2O)cc1N1CCC(C(=O)N2CC3(CN(C(=O)CC4CCN(c5ccc6c(c5)C(=O)N(C5CCC(=O)NC5=O)C6=O)CC4)C3)C2)(c2ccccc2)CC1]

The title compound was prepared in analogy to example 121 step d using Ligase 24 as a yellow solid (18.5 mg, 20.6 μmol, 57% yield), formate salt. MS (ESI): 852.7 ([M+H]⁺).

### Example 123

### 3-(4-(1-(4-(6-(1-(3-amino-6-(2-hydroxyphenyl)pyridazin-4-yl)-4-phenylpiperidine-4-carbonyl)-2,6-diazaspiro[3.3]heptan-2-yl)-4-oxobutyl)piperidin-4-yl)phenoxy)piperidine-2,6-dione

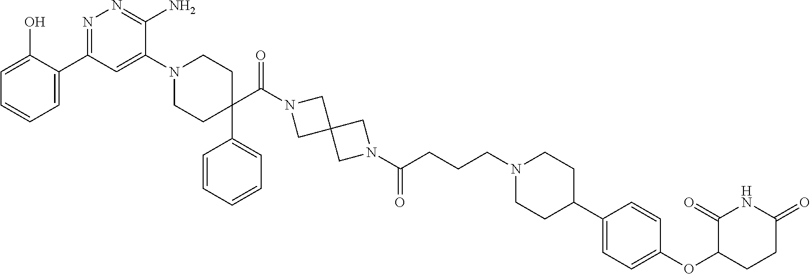 [SMILES: Nc1nnc(-c2ccccc2O)cc1N1CCC(C(=O)N2CC3(CN(C(=O)CCCN4CCC(c5ccc(OC6CCC(=O)NC6=O)cc5)CC4)C3)C2)(c2ccccc2)CC1]

The title compound was prepared in analogy to example 121 step d using Ligase 39 as a yellow solid (9.5 mg, 9.79 μmol, 27.1% yield), formate salt. MS (ESI): 827.7 ([M+H]⁺).

### Example 124

### 1-[3-amino-6-(2-hydroxyphenyl)pyridazin-4-yl]-N-[[1-[[l-[2-(2,6-dioxo-3-piperidyl)-1,3-dioxo-isoindolin-5-yl]-4-piperidyl]methyl]-4-piperidyl]methyl]-3-phenyl-pyrrolidine-3-carboxamide

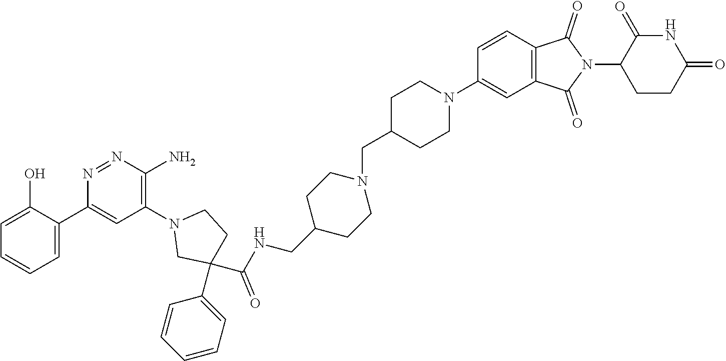

### a) benzyl 4-(aminomethyl)piperidine-1-carboxylate

To a cooled (0° C.) solution of benzyl 4-(((tert-butoxycarbonyl)amino)methyl)piperidine-1-carboxylate (3.45 g, 9.9 mmol, 1.0 eq) in DCM (10 mL) was added 4M HCl in dioxane (7.43 ml, 29.7 mmol, 3.0 eq). The reaction mixture was allowed to reach room temperature and stirred for 4 h. The reaction mixture was filtered and the solid was dried in vacuo to afford the title compound (2.77 g, 9.7 mmol, 98% yield) as a white solid, hydrochloride salt. MS (ESI): 249.3 ([M+H]⁺).

### b) benzyl 4-[[(1-tert-butoxycarbonyl-3-phenyl-pyrrolidine-3-carbonyl)amino]methyl]piperidine-1-carboxylate

To a solution of 1-(tert-butoxycarbonyl)-3-phenylpyrrolidine-3-carboxylic acid (300 mg, 1.03 mmol, 1.0 eq), HATU (470 mg, 1.24 mmol, 1.2 eq) and DIPEA (665 mg, 899 μL, 5.15 mmol, 5.0 eq) in DMF (4 mL) was added benzyl 4-(aminomethyl)piperidine-1-carboxylate hydrochloride (323 mg, 1.13 mmol, 1.1 eq). The reaction mixture was stirred at room temperature for 4 h. The reaction mixture was concentrated in vacuo. The crude material was purified on silica column (heptane/EtOAc 0-100%) to afford the title compound (460 mg, 886 μmol, 86% yield) as a light brown solid. MS (ESI): 422.4 ([M+H-Boc]⁺).

### c) benzyl 4-[[(3-phenylpyrrolidine-3-carbonyl)amino]methyl]piperidine-1-carboxylate

To a cooled (0° C.) solution of benzyl 4-((1-(tert-butoxycarbonyl)-3-phenylpyrrolidine-3-carboxamido)methyl)piperidine-1-carboxylate (460 mg, 882 μmol, 1.0 eq) in DCM (6 mL) was added 4M HCl in dioxane (661 μL, 2.65 mmol, 3.0 eq). The reaction mixture was allowed to reach room temperature and stirred for 16 h. The reaction mixture was concentrated in vacuo to afford the title compound (385 mg, 840 μmol, 95% yield) as a white solid, hydrochloride salt. MS (ESI): 422.4 ([M+H]⁺).

### d) benzyl 4-[[[1-(3-amino-6-chloro-pyridazin-4-yl)-3-phenyl-pyrrolidine-3-carbonyl]amino]methyl]piperidine-1-carboxylate

To a solution of 4-bromo-6-chloropyridazin-3-amine (238 mg, 1.14 mmol, 1.1 eq) and benzyl 4-((3-phenylpyrrolidine-3-carboxamido)methyl)piperidine-1-carboxylate, hydrochloride salt (475 mg, 1.04 mmol, 1.0 eq) in DMSO (5 mL) was added potassium carbonate (717 mg, 5.19 mmol, 5.0 eq) and the reaction mixture was stirred at 110° C. for 16 h. The reaction mixture was cooled to room temperature, poured in saturated NaHCO₃ and extracted with EtOAc. The organic combined organic layers were washed with water and brine, dried over Na₂SO₄ and concentrated. The crude material was purified on silica column (DCM/MeOH 0-10%) to afford the title compound (360 mg, 655 μmol, 63% yield) as a brown solid. MS (ESI): 549.4 ([M+H]⁺).

### e) benzyl 4-[[[1-[3-amino-6-(2-hydroxyphenyl)pyridazin-4-yl]-3-phenyl-pyrrolidine-3-carbonyl]amino]methyl]piperidine-1-carboxylate

A suspension of benzyl 4-((1-(3-amino-6-chloropyridazin-4-yl)-3-phenylpyrrolidine-3-carboxamido)methyl)piperidine-1-carboxylate (360 mg, 656 μmol, 1.0 eq), (2-hydroxyphenyl)boronic acid (226 mg, 1.64 mmol, 2.5 eq), potassium carbonate (272 mg, 1.97 mmol, 3.0 eq) and RuPhos Pd G3 (27.4 mg, 32.8 μmol, 0.05 eq) in a mixture of degassed dioxane (3 mL) and water (0.3 mL) was stirred at 110° C. for 4 h under argon. The reaction mixture was poured in saturated NaHCO₃ and extracted with EtOAc. The organic layers were combined, washed with water and brine. The organic layers were dried over Na₂SO₄ and concentrated in vacuo. The crude material was purified on silica column (DCM/MeOH 0-10%) to afford the title compound (295 mg, 485 μmol, 74% yield) as a light brown solid. MS (ESI): 607.5 ([M+H]⁺).

### f) 1-[3-amino-6-(2-hydroxyphenyl)pyridazin-4-yl]-3-phenyl-N-(4-piperidylmethyl)pyrrolidine-3-carboxamide

To a solution of benzyl 4-((1-(3-amino-6-(2-hydroxyphenyl)pyridazin-4-yl)-3-phenylpyrrolidine-3-carboxamido)methyl)piperidine-1-carboxylate (200 mg, 330 μmol, 1.0 eq) in methanol (4 mL) was added 10% palladium on charcoal (35.1 mg, 33 μmol, 0.1 eq). The reaction mixture was vigorously stirred at room temperature for 5 h under H₂ (baloon). The catalyst was collected by filtration and washed with methanol. The filtrate was concentrated to afford the title compound (140 mg, 297 μmol, 90% yield) as a white solid. MS (ESI): 473.4 ([M+H]⁺).

### g) 1-[3-amino-6-(2-hydroxyphenyl)pyridazin-4-yl]-N-[[1-[[1-[2-(2,6-dioxo-3-piperidyl)-1,3-dioxo-isoindolin-5-yl]-4-piperidyl]methyl]-4-piperidyl]methyl]-3-phenyl-pyrrolidine-3-carboxamide

The title compound was prepared in analogy to example 100 using Ligase 33 as a yellow solid (5.5 mg, 6.31 μmol, 14% yield), formate salt. MS (ESI): 826.7 ([M+H]⁺).

### Example 125

### rac-5-[4-[4-[4-[1-[3-amino-6-(2-hydroxyphenyl)pyridazin-4-yl]azetidin-3-yl]oxy-2-chloro-benzoyl]piperazine-1-carbonyl]-1-piperidyl]-2-(2,6-dioxo-3-piperidyl)isoindoline-1,3-dione

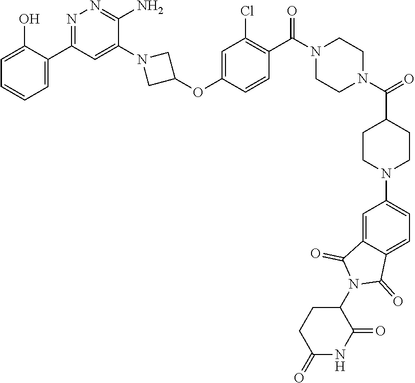 [SMILES: Nc1nnc(-c2ccccc2O)cc1N1CC(Oc2ccc(C(=O)N3CCN(C(=O)C4CCN(c5ccc6c(c5)C(=O)N(C5CCC(=O)NC5=O)C6=O)CC4)CC3)c(Cl)c2)C1]

To a stirred solution of [4-[1-[3-amino-6-(2-hydroxyphenyl)pyridazin-4-yl]azetidin-3-yl]oxy-2-chloro-phenyl]-piperazin-1-yl-methanone (30 mg, 62.4 μmol, 1.0 eq) in DMSO (0.5 mL) at room temperature was added Ligase 15 (24 mg, 62.4 μmol, 1.0 eq). The reaction mixture was stirred at room temperature for 1 h. The purification of the crude material was done by prep-HPLC to afford the title compound (19.5 mg, 21.1 μmol, 34% yield) as a yellow saslt, bis-(2,2,2-trifluoroacetic acid)salt. MS (ESI): 848.6 (³⁵Cl[M+H]⁺).

### Example 126

### 2-(2,6-dioxo-3-piperidyl)-4-[1-[1-[9-[4-[4-[rac-(3S)-3-[3-amino-6-(2-hydroxyphenyl)pyridazin-4-yl]oxy-1-piperidyl]phenyl]piperazin-1-yl]nonyl]triazol-4-yl]ethoxy]isoindoline-1,3-dione

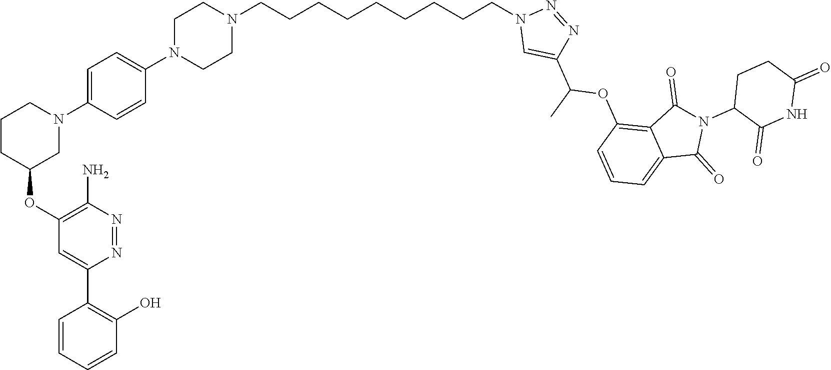 [SMILES: CC(Oc1cccc2c1C(=O)N(C1CCC(=O)NC1=O)C2=O)c1cn(CCCCCCCCCN2CCN(c3ccc(N4CCC[C@H](Oc5cc(-c6ccccc6O)nnc5N)C4)cc3)CC2)nn1]

To a solution of (S)-2-[6-amino-5-[[1-(4-piperazin-1-ylphenyl)-3-piperidyl]oxy]pyridazin-3-yl]phenol (40.0 mg, 88 μmol) and Ligase 45 (45 mg, 88 μmol) in THF (5 mL) was added dibutyltin dichloride (27 mg, 89 μmol, 19 μL) followed by the addition of trimethyl(phenyl)silane (12 mg, 107 μmol) and the reaction mixture was heated to 80° C. for 16 h. The reaction mixture was cooled to room temperature, diluted with water and extracted with EtOAc. The organic layer was washed with water, brine solution, dried over anhydrous sodium sulfate, filtered and concentrated under reduced pressure. The crude residue was purified by prep-HPLC to afford the title compound (6.2 mg, 6.25 μmol, 7% yield) as an off-white solid. MS (ESI): 940.3 ([M+H]⁺).

### Example 127

### 5-(4-(4-(3-(7-(3-amino-6-(2-hydroxyphenyl)pyridazin-4-yl)-4,7-diazaspiro[2.5]octan-4-yl)benzyl)piperazine-1-carbonyl)piperidin-1-yl)-2-(2,6-dioxopiperidin-3-yl)isoindoline-1,3-dione

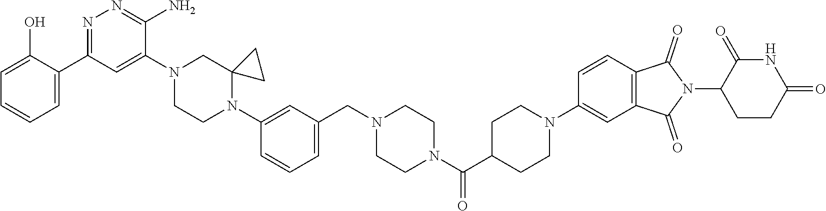

### a) benzyl 4-[(3-bromophenyl)methyl]piperazine-1-carboxylate

To a solution of 3-bromobenzaldehyde (6.3 mL, 54 mmol, 1 eq), 1-Cbz-piperazine (17857 mg, 81 mmol, 1.5 eq), AcOH (1621 mg, 27 mmol, 0.5 eq) in methanol (100 mL) was added NaBH3CN (6792 mg, 108 mmol, 2 eq) at 25° C., the reaction mixture was stirred at 25° C. for 12 h. The reaction mixture was concentrated, purified by prep-HPLC (FA) to afford the title compound (15000 mg, 38 mmol, 71% yield) as an colorless oil.

### b) tert-butyl 4-[3-[(4-benzyloxycarbonylpiperazin-1-yl)methyl]phenyl]-4,7-diazaspiro[2.5]octane-7-carboxylate

A mixture of benzyl 4-[(3-bromophenyl)methyl]piperazine-1-carboxylate (2000 mg, 5.14 mmol, 1 eq), tert-butyl 4,7-diazaspiro[2.5]octane-7-carboxylate (1308 mg, 6.17 mmol, 1.2 eq), t-BuONa (740 mg, 7.71 mmol, 1.5 eq), Pd(OAc)2 (57.6 mg, 0.26 mmol, 0.05 eq), RuPhos (239 mg, 0.51 mmol, 0.1 eq), in 1,4-dioxane (50 mL) was stirred at 100° C. for 12 h under N2 atmosphere. The reaction mixture was concentrated purified on silica column (heptane/EtOAc 20-50%) to give the title compound (1000 mg, 1.92 mmol, 37% yield) as an yellow oil.

### c) benzyl 4-[[3-(4,7-diazaspiro[2.5]octan-4-yl)phenyl]methyl]piperazine-1-carboxylate

A mixture of tert-butyl 4-[3-[(4-benzyloxycarbonylpiperazin-1-yl)methyl]phenyl]-4,7-diazaspiro[2.5]octane-7-carboxylate (1000 mg, 1.92 mmol, 1 eq) in TFA (5.0 mL, 1.92 mmol, 1 eq) and DCM (20 mL) was stirred at 1 h for 25° C. The reaction mixture was concentrated to give the title compound (800 mg, 1.9 mmol, 99% yield) as an yellow oil.

### d) benzyl 4-[[3-[7-(3-amino-6-chloro-pyridazin-4-yl)-4,7-diazaspiro[2.5]octan-4-yl]phenyl]methyl]piperazine-1-carboxylate

A mixture of benzyl 4-[[3-(4,7-diazaspiro[2.5]octan-4-yl)phenyl]methyl]piperazine-1-carboxylate (800 mg, 1.9 mmol, 1 eq), 4-bromo-6-chloro-pyridazin-3-amine (475 mg, 2.28 mmol, 1.2 eq) Et3N (962 mg, 9.51 mmol, 5 eq) in DMF (20 mL) was stirred at 100° C. for 12 h. The reaction mixture was purified by prep-HPLC(FA) to give the title compound (600 mg, 1.09 mmol, 57% yield) as an yellow oil.

### e) benzyl 4-[[3-[7-[3-amino-6-(2-hydroxyphenyl)pyridazin-4-yl]-4,7-diazaspiro[2.5]octan-4-yl]phenyl]methyl]piperazine-1-carboxylate

A mixture of benzyl 4-[[3-[7-(3-amino-6-chloro-pyridazin-4-yl)-4,7-diazaspiro[2.5]octan-4-yl]phenyl]methyl]piperazine-1-carboxylate (600 mg, 1.09 mmol, 1 eq), 2-hydroxyphenylboronic acid (377 mg, 2.74 mmol, 2.5 eq), K2CO3 (529 mg, 3.83 mmol, 3.5 eq), Ruphos Pd G3 (94.5 mg, 0.11 mmol, 0.1 eq) in 1,4-dioxane (10 mL) and water (1 mL) was stirred at 90° C. for 2 h under N2 atmosphere. The reaction mixture was filtered and the filtrate was concentrated and purified by prep-HPLC (FA) to give (500 mg, 0.83 mmol, 75% yield) as a yellow oil.

### f) 2-[6-amino-5-[4-[3-(piperazin-1-ylmethyl)phenyl]-4,7-diazaspiro[2.5]octan-7-yl]pyridazin-3-yl]phenol

A mixture of benzyl 4-[[3-[7-[3-amino-6-(2-hydroxyphenyl)pyridazin-4-yl]-4,7-diazaspiro[2.5]octan-4-yl]phenyl]methyl]piperazine-1-carboxylate (500 mg, 0.83 mmol, 1 eq) Pd/C (175 mg, 1.65 mmol, 2 eq), in methanol (5 mL) and DCM (3 mL) was stirred at 25° C. for 12 h under H2 atmosphere. The reaction mixture was filtered and the filtrate was concentrated and purified by prep-HPLC (TFA) to give the title compound (131.1 mg, 0.28 mmol, 31% yield) as yellow solid.

### g) 5-(4-(4-(3-(7-(3-amino-6-(2-hydroxyphenyl)pyridazin-4-yl)-4,7-diazaspiro[2.5]octan-4-yl)benzyl)piperazine-1-carbonyl)piperidin-1-yl)-2-(2,6-dioxopiperidin-3-yl)isoindoline-1,3-dione

Ligase 15 (14.7 mg, 38.2 μmol, 1.2 eq) was dissolved in dry DMF (400 μL). DIPEA (10.3 mg, 13.9 μL, 79.5 μmol, 2.5 eq) and HATU (15.7 mg, 41.3 μmol, 1.3 eq) were added and the mixture was stirred at room temperature for 10 min. 2-(6-amino-5-(4-(3-(piperazin-1-ylmethyl)phenyl)-4,7-diazaspiro[2.5]octan-7-yl)pyridazin-3-yl)phenol (15 mg, 31.8 μmol, 1.0 eq) was added and the mixture was stirred at room temperature for 2 h. The reaction mixture was purified by prep-HPLC to afford the title compound (6.7 mg, 7.04 μmol, 22% yield) as a yellow salt, formate salt. MS (ESI): 839.6 ([M+H]⁺).

### Example 128

### 5-(4-(3-(4-(3-(7-(3-amino-6-(2-hydroxyphenyl)pyridazin-4-yl)-4,7-diazaspiro[2.5]octan-4-yl)benzyl)piperazin-1-yl)-3-oxopropyl)piperidin-1-yl)-2-(2,6-dioxopiperidin-3-yl)isoindoline-1,3-dione

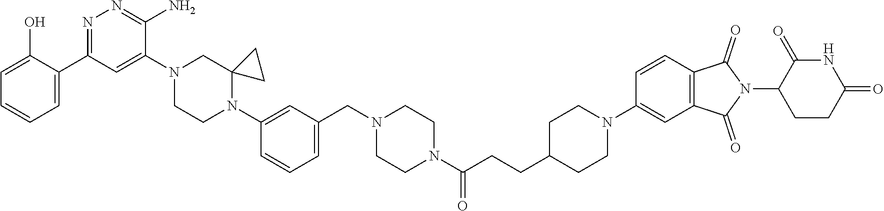

The title compound was prepared in analogy to example 127 step g using Ligase 12 as a yellow solid (8 mg, 7.89 μmol, 24% yield), formate salt. MS (ESI): 867.7 ([M+H]⁺).

### Example 129

### 5-(2-(4-(3-(7-(3-amino-6-(2-hydroxyphenyl)pyridazin-4-yl)-4,7-diazaspiro[2.5]octan-4-yl)benzyl)piperazin-1-yl)-2-oxoethoxy)-2-(2,6-dioxopiperidin-3-yl)isoindoline-1,3-dione

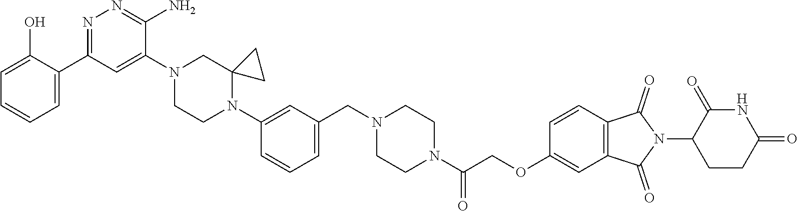

The title compound was prepared in analogy to example 127 step g using Ligase 48 as a yellow solid (6.6 mg, 7.7 μmol, 24% yield), formate salt. MS (ESI): 786.6 ([M+H]⁺).

### Example 130

### 5-(4-(2-(4-(3-(7-(3-amino-6-(2-hydroxyphenyl)pyridazin-4-yl)-4,7-diazaspiro[2.5]octan-4-yl)benzyl)piperazin-1-yl)-2-oxoethyl)piperidin-1-yl)-2-(2,6-dioxopiperidin-3-yl)isoindoline-1,3-dione

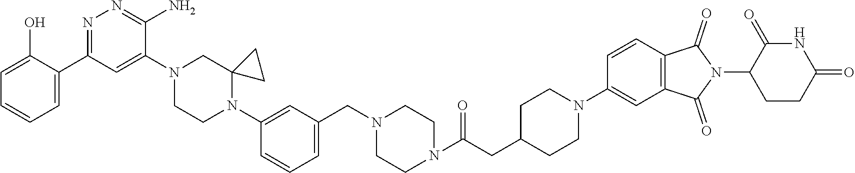

The title compound was prepared in analogy to example 127 step g using Ligase 24 as a yellow solid (7.4 mg, 7.9 μmol, 24% yield), formate salt. MS (ESI): 853.7 ([M+H]⁺).

### Example 131

### rac-5-[4-[4-[[6-[3-[3-amino-6-(2-hydroxyphenyl)pyridazin-4-yl]-3,8-diazabicyclo[3.2.1]octan-8-yl]-2-pyridyl]methyl]piperazine-1-carbonyl]-1-piperidyl]-2-(2,6-dioxo-3-piperidyl)isoindoline-1,3-dione

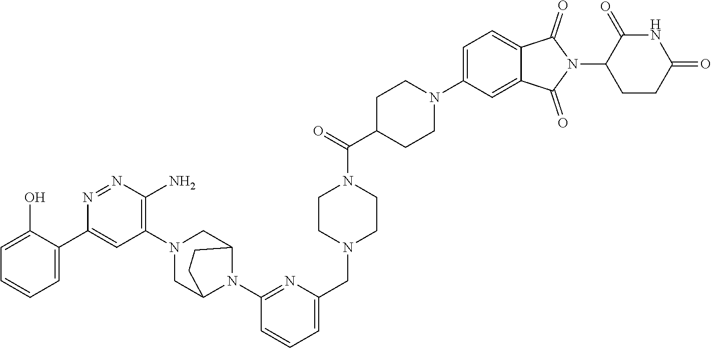

### a) benzyl 4-[(6-bromo-2-pyridyl)methyl]piperazine-1-carboxylate

A solution of 2-bromo-6-(bromomethyl)pyridine (1.32 g, 5.26 mmol, 1.0 eq) and benzyl piperazine-1-carboxylate (2.32 g, 10.5 mmol, 2.0 eq) in THF (10 mL) was stirred for 20 h. A white solution resulted. The reaction mixture was partitioned between EtOAc and 0.5 M aqueous sodium hydroxide solution. The layers were separated. The aqueous layer was extracted with EtOAc. The combined organic layers were washed with brine, dried over anhydrous sodium sulfate, and concentrated in vacuo. The crude material was purified on silica column (heptane/EtOAc 0-50%) to afford the title compound (1.72 g, 4.19 mmol, 80% yield) as a colourless oil. MS (ESI): 391.5 ([M+H]⁺).

### b) tert-butyl 8-[6-[(4-benzyloxycarbonylpiperazin-1-yl)methyl]-2-pyridyl]-3,8-diazabicyclo[3.2.1]octane-3-carboxylate

To a stirred suspension of benzyl 4-[(6-bromo-2-pyridyl)methyl]piperazine-1-carboxylate (241 mg, 618 μmol, 1.0 eq) and tert-butyl 3,8-diazabicyclo[3.2.1]octane-3-carboxylate (138 mg, 648 μmol, 1.05 eq) in tert-BuOH (3 mL) at room temperature was added K₂CO₃ (171 mg, 1.24 mmol, 2.0 eq). The reaction was degassed with argon for 5 min. RuPhos Pd G3 (51.6 mg, 61.8 μmol, 0.1 eq) was then added. The reaction mixture was stirred at 120° C. for 5 h. The reaction mixture was poured into EtOAc and washed sequentially with water and with brine. The organic layer was dried over Na₂SO₄ and concentrated in vacuo. The crude material was purified on silica column (heptane/EtOAc 0-60%) to afford the title compound (143 mg, 195 μmol, 32% yield) as an off-white solid. MS (ESI): 522.5 ([M+H]⁺).

### c) benzyl 4-[[6-(3,8-diazabicyclo[3.2.1]octan-8-yl)-2-pyridyl]methyl]piperazine-1-carboxylate

To a stirred solution of tert-butyl 8-[6-[(4-benzyloxycarbonylpiperazin-1-yl)methyl]-2-pyridyl]-3,8-diazabicyclo[3.2.1]octane-3-carboxylate (1.513 g, 2.03 mmol, 1.0 eq) in 1,4-dioxane (6.77 mL) at room temperature was added 4 M hydrogen chloride solution in 1,4-dioxane (5.08 mL, 20.3 mmol, 10.0 eq). The reaction mixture was stirred for 15 h. The reaction mixture was partitioned between EtOAc and 0.5 M aqueous hydrochloric acid. The aqueous layer was washed with EtOAc. The layers were separated. The pH of the aqueous layer was set to 14 by addition of 5 N aqueous NaOH and the mixture was extracted with EtOAc/THF (1:1). The combined organic layers were dried over anhydrous sodium sulfate and concentrated in vacuo to afford the title compound (1.097 g, 1.98 mmol, 97% yield) as a yellow oil. MS (ESI): 422.4 ([M+H]⁺).

### d) benzyl 4-[[6-[3-(3-amino-6-chloro-pyridazin-4-yl)-3,8-diazabicyclo[3.2.1]octan-8-yl]-2-pyridyl]methyl]piperazine-1-carboxylate

To a stirred suspension of benzyl 4-[[6-(3,8-diazabicyclo[3.2.1]octan-8-yl)-2-pyridyl]methyl]piperazine-1-carboxylate (1.076 g, 2.55 mmol, 1.0 eq) and 4-bromo-6-chloropyridazin-3-amine (692 mg, 3.32 mmol, 1.3 eq) in DMSO (6.38 mL) at room temperature was added K₂CO₃ (1.76 g, 12.8 mmol, 5.0 eq). The reaction mixture was heated at 110° C. for 15 h. The reaction mixture was partitioned between EtOAc and water. The layers were separated. The aqueous layer was extracted with EtOAc. The combined organic layers were washed with brine, dried over anhydrous sodium sulfate, and concentrated in vacuo. The crude material was purified on silica column (DCM/MeOH 0-10%) and then on amine modified silica column (DCM/MeOH 0-5%). to afford the title compound (895 mg, 1.45 mmol, 57% yield) as a yellow oil. MS (ESI): 549.5 (³⁵Cl[M+H]⁺).

### e) benzyl 4-[[6-[3-[3-amino-6-(2-hydroxyphenyl)pyridazin-4-yl]-3,8-diazabicyclo[3.2.1]octan-8-yl]-2-pyridyl]methyl]piperazine-1-carboxylate and 2-[6-amino-5-[8-[6-(piperazin-1-ylmethyl)-2-pyridyl]-3,8-diazabicyclo[3.2.1]octan-3-yl]pyridazin-3-yl]phenol

To a stirred suspension of benzyl 4-[[6-[3-(3-amino-6-chloro-pyridazin-4-yl)-3,8-diazabicyclo[3.2.1]octan-8-yl]-2-pyridyl]methyl]piperazine-1-carboxylate (895 mg, 1.63 mmol, 1.0 eq) and (2-hydroxyphenyl)boronic acid (562 mg, 4.08 mmol, 2.5 eq) in 1,4-dioxane (36 mL) and water (0.9 mL) at room temperature was added K₂CO₃ (788 mg, 5.71 mmol, 3.5 eq). The reaction mixture was degassed with argon for 10 min. RuPhos Pd G3 (136 mg, 163 μmol, 0.1 eq) was added. The reaction mixture was heated at 90° C. for 2 days. The reaction mixture was partitioned between EtOAc/THF and water. The layers were separated. The aqueous layer was extracted with EtOAc/THF. The combined organic layers were washed with brine, dried over anhydrous sodium sulfate, and concentrated in vacuo. The crude material was purified on silica column (DCM/MeOH 0-10%) to afford the title compound 1 (235 mg, 387 μmol, 24% yield) as an orange solid, MS(ESI): 607.5 ([M+H]⁺), and the title compound 2 (648 mg, 1.37 mmol, 84% yield) as a light brown solid. MS (ESI): 500.4 ([M+H]⁺).

### f) rac-5-[4-[4-[[6-[3-[3-amino-6-(2-hydroxyphenyl)pyridazin-4-yl]-3,8-diazabicyclo[3.2.1]octan-8-yl]-2-pyridyl]methyl]piperazine-1-carbonyl]-1-piperidyl]-2-(2,6-dioxo-3-piperidyl)isoindoline-1,3-dione;2,2,2-trifluoroacetic acid

To a stirred solution of 2-[6-amino-5-[8-[6-(piperazin-1-ylmethyl)-2-pyridyl]-3,8-diazabicyclo[3.2.1]octan-3-yl]pyridazin-3-yl]phenol 30 mg, 63.5 μmol, 1.0 eq), Ligase 15 (24.5 mg, 63.5 μmol, 1.0 eq) and HATU (53.1 mg, 140 μmol, 2.2 eq) in DMSO (0.5 mL) at room temperature was added DIPEA (24.6 mg, 33.3 μL, 190 μmol, 3.0 eq). The reaction mixture was stirred for 2 h. The purification was done by prep-HPLC to afford the title compound (21.5 mg, 20.1 μmol, 32% yield) as a yellow solid, bis-(2,2,2-trifluoroacetic acid)salt. MS (ESI): 840.7 ([M+H]⁺).

### Example 132

### rac-5-[4-[4-[4-[1-[3-amino-6-(2-hydroxyphenyl)pyridazin-4-yl]azetidin-3-yl]oxy-2-(trifluoromethyl)benzoyl]piperazine-1-carbonyl]-1-piperidyl]-2-(2,6-dioxo-3-piperidyl)isoindoline-1,3-dione

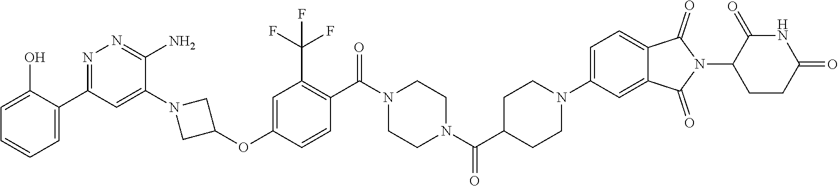

To a stirred solution of [4-[1-[3-amino-6-(2-hydroxyphenyl)pyridazin-4-yl]azetidin-3-yl]oxy-2-(trifluoromethyl)phenyl]-piperazin-1-yl-methanone (30 mg, 58.3 μmol, 1.0 eq), Ligase 15 (22.5 mg, 58.3 μmol, 1.0 eq) and HATU (44.3 mg, 117 μmol, 2.0 eq) in DMSO (0.5 mL) was added DIPEA (22.6 mg, 30.6 μL, 175 μmol, 3.0 eq). The reaction mixture was stirred at room temperature for 2 h. The reaction mixture was partitioned between EtOAc/THF (1:1) and water. The layers were separated. The aqueous layer was extracted with EtOAc. The combined organic layers were washed with brine, dried over anhydrous sodium sulfate and concentrated in vacuo. The crude material was purified by prep-RP-HPLC to afford the title compound (7.1 mg, 7.09 μmol, 12% yield) as a yellow solid, 2,2,2-trifluoroacetate salt. MS (ESI): 882.7 ([M+H]⁺).

### Example 133

### rac-5-[4-[3-[4-[[6-[3-[3-amino-6-(2-hydroxyphenyl)pyridazin-4-yl]-3,8-diazabicyclo[3.2.1]octan-8-yl]-2-pyridyl]methyl]piperazin-1-yl]-3-oxo-propyl]-1-piperidyl]-2-(2,6-dioxo-3-piperidyl)isoindoline-1,3-dione

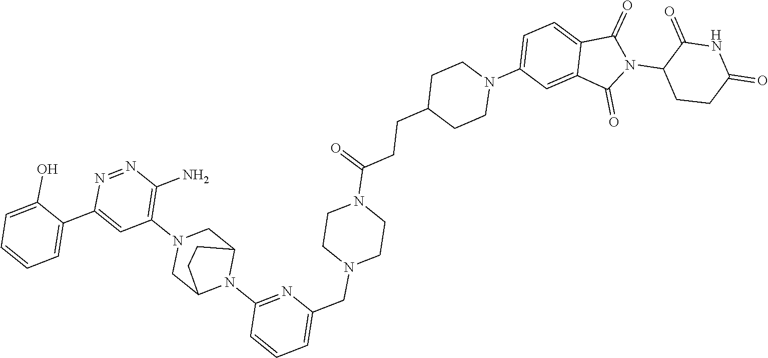

To a stirred solution of 2-[6-amino-5-[8-[6-(piperazin-1-ylmethyl)-2-pyridyl]-3,8-diazabicyclo[3.2.1]octan-3-yl]pyridazin-3-yl]phenol (30 mg, 63.5 μmol, 1.0 eq), Ligase 12 (26.2 mg, 63.5 μmol, 1.0 eq) and HATU (53.1 mg, 140 μmol, 2.2 eq) in DMSO (0.5 mL) at room temperature was added DIPEA (24.6 mg, 33.3 μL, 190 μmol, 3.0 eq). The reaction mixture was stirred for 2 hand purified by prep-HPLC to afford the title compound (8.1 mg, 7.17 μmol, 11% yield) as a yellow solid, bis-(2,2,2-trifluoroacetic acid)salt. MS: 869.6 ([M+H]1).

### Example 134

### rac-1-[[3-[3-[3-amino-6-(2-hydroxyphenyl)pyridazin-4-yl]-3,8-diazabicyclo[3.2.1]octan-8-yl]phenyl]methyl]-N-[1-[2-(2,6-dioxo-3-piperidyl)-1,3-dioxo-isoindolin-5-yl]azetidin-3-yl]piperidine-4-carboxamide

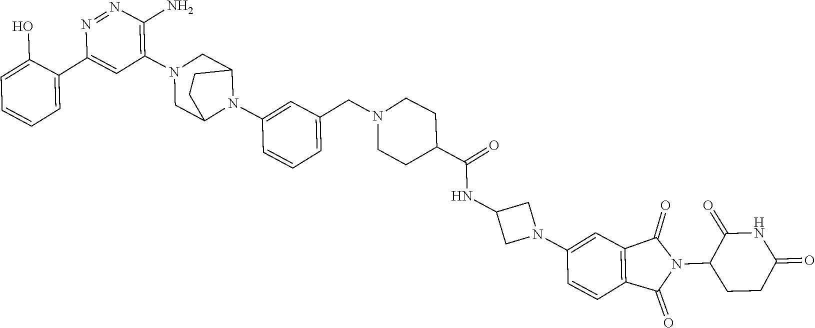

To a stirred solution of 1-[[3-[3-[3-amino-6-(2-hydroxyphenyl)pyridazin-4-yl]-3,8-diazabicyclo[3.2.1]octan-8-yl]phenyl]methyl]piperidine-4-carboxylic acid (60 mg, 76.9 μmol, 1.0 eq), Ligase 59 (25.3 mg, 76.9 μmol, 1.0 eq) and DIPEA (49.7 mg, 67.2 μL, 385 μmol, 5.0 eq) in DMSO (0.6 mL) at room temperature was added HATU (58.5 mg, 154 μmol, 2.0 eq). The reaction mixture was stirred at room temperature for 1 h. The crude material was purified by prep-HPLC to afford the title compound (24 mg, 21.7 μmol, 24% yield) as a yellow solid, bis-(2,2,2-trifluoroacetic acid)salt. MS (ESI): 825.4 ([M+H]⁺).

### Example 135

### 2-(2,6-dioxo-3-piperidyl)-4-[[1-[[1-[5-[4-[4-[rac-(3S)-3-[3-amino-6-(2-hydroxyphenyl)pyridazin-4-yl]oxy-1-piperidyl]phenyl]piperazin-1-yl]pentanoyl]-4-piperidyl]methyl]triazol-4-yl]methoxy]isoindoline-1,3-dione

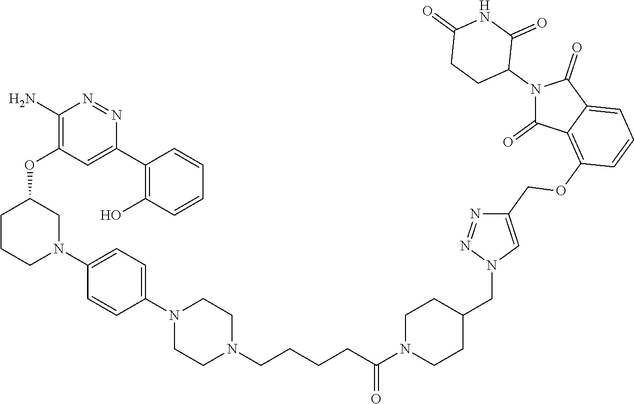 [SMILES: Nc1nnc(-c2ccccc2O)cc1O[C@H]1CCCN(c2ccc(N3CCN(CCCCC(=O)N4CCC(Cn5cc(COc6cccc7c6C(=O)N(C6CCC(=O)NC6=O)C7=O)nn5)CC4)CC3)cc2)C1]

To a solution of 5-[4-[4-[(3S)-3-[3-amino-6-(2-hydroxyphenyl)pyridazin-4-yl]oxy-1-piperidyl]phenyl]piperazin-1-yl]pentanoic acid (40 mg, 73 μmol) and Ligase 61 (33 mg, 58 μmol, TFA) in dimethylformamide (0.4 mL) was added HATU (41 mg, 109 μmol) followed by DIPEA (28 mg, 219 μmol, 38 μL) and the reaction mixture was stirred at room temperature for 16 h. The reaction mixture was diluted with water and extracted with EtOAc. The volatiles were removed. The crude residue was purified by prep-HPLC to afford the title compound (3.5 mg, 3.51 μmol, 5% yield) as an off-white solid. MS (ESI): 981.1 ([M+H]⁺).

### Example 136

### 4-[[5-[9-[4-[4-[3-[3-amino-6-(2-hydroxyphenyl)pyridazin-4-yl]oxy-1-piperidyl]phenyl]piperazin-1-yl]nonyl]-2-pyridyl]methylamino]-2-(2,6-dioxo-3-piperidyl)isoindoline-1,3-dione

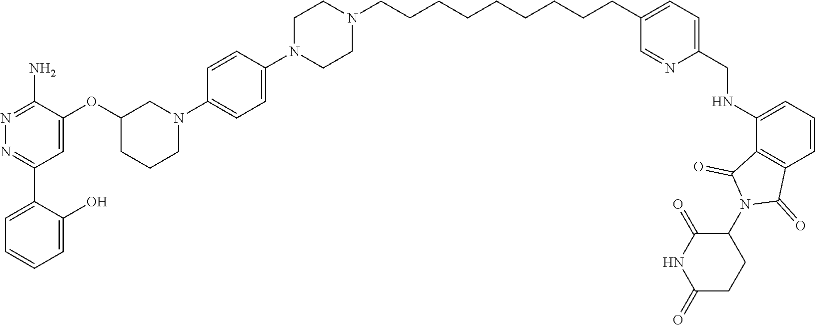 [SMILES: Nc1nnc(-c2ccccc2O)cc1OC1CCCN(c2ccc(N3CCN(CCCCCCCCCc4ccc(CNc5cccc6c5C(=O)N(C5CCC(=O)NC5=O)C6=O)nc4)CC3)cc2)C1]

To a solution of 2-[6-amino-5-[[1-(4-piperazin-1-ylphenyl)-3-piperidyl]oxy]pyridazin-3-yl]phenol (25 mg, 56 μmol) and Ligase 62 (28 mg, 56 μmol) in THF (5 mL) was added dibutyltin dichloride (17 mg, 55.99 μmol, 12 μL) followed by the addition of trimethyl(phenyl)silane (7 mg, 67 μmol) and the reaction mixture was heated to 80° C. for 16 h. The reaction mixture was cooled to room temperature, diluted with water and extracted with EtOAc. The organic layer was washed with water, brine solution, dried over anhydrous sodium sulfate, filtered and concentrated under reduced pressure. The crude residue was purified by prep-HPLC to afford the title compound (2.33 mg, 2.23 μmol, 4% yield). MS (ESI): 935.1 ([M+H]⁺).

### Example 137

### 2-(2,6-dioxo-3-piperidyl)-4-[[1-[5-oxo-5-[3-[[4-[4-[rac-(3S)-3-[3-amino-6-(2-hydroxyphenyl)pyridazin-4-yl]oxy-1-piperidyl]phenyl]piperazin-1-yl]methyl]azetidin-1-yl]pentyl]triazol-4-yl]methoxy]isoindoline-1,3-dione

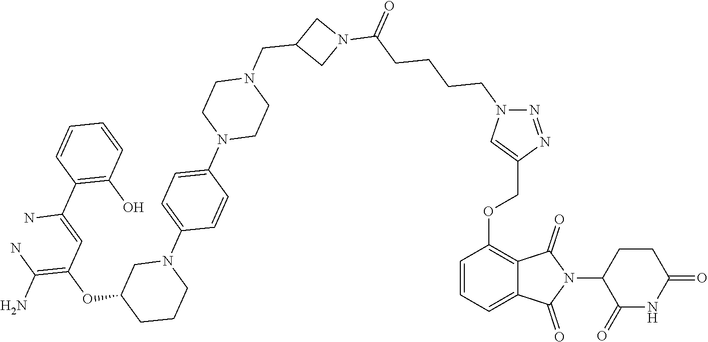 [SMILES: NC(N)=C(/C=C(\N)c1ccccc1O)O[C@H]1CCCN(c2ccc(N3CCN(CC4CN(C(=O)CCCCn5cc(COc6cccc7c6C(=O)N(C6CCC(=O)NC6=O)C7=O)nn5)C4)CC3)cc2)C1]

To a solution of Ligase 63 (35 mg, 61 μmol, TFA) and 2-[6-amino-5-[[(3S)-1-[4-[4-(azetidin-3-ylmethyl)piperazin-1-yl]phenyl]-3-piperidyl]oxy]pyridazin-3-yl]phenol (38 mg, 61 μmol, TFA salt) in DMF (1 mL) was added HATU (41 mg, 109 μmol) followed by DIPEA (40 mg, 307 μmol, 53 μL) and the reaction mixture was stirred at room temperature for 16 h. The reaction mixture was diluted with water and extracted with EtOAc. The volatiles were removed. The crude residue was purified by prep-HPLC to afford the title compound (4 mg, 3.89 μmol, 6% yield) as an off-white solid. MS (ESI): 953.3 ([M+H]⁺).

### Example 138

### 2-(2,6-dioxo-3-piperidyl)-4-[[1-[1-[5-[4-[4-[rac-(3S)-3-[3-amino-6-(2-hydroxyphenyl)pyridazin-4-yl]oxy-1-piperidyl]phenyl]piperazin-1-yl]pentanoyl]-4-piperidyl]triazol-4-yl]methoxy]isoindoline-1,3-dione

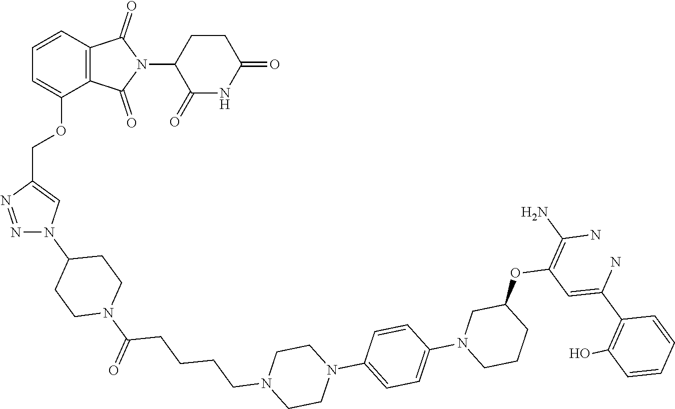 [SMILES: NC(N)=C(/C=C(\N)c1ccccc1O)O[C@H]1CCCN(c2ccc(N3CCN(CCCCC(=O)N4CCC(n5cc(COc6cccc7c6C(=O)N(C6CCC(=O)NC6=O)C7=O)nn5)CC4)CC3)cc2)C1]

To a solution 5-[4-[4-[(3S)-3-[3-amino-6-(2-hydroxyphenyl)pyridazin-4-yl]oxy-1-piperidyl]phenyl]piperazin-1-yl]pentanoic acid (20 mg, 0.036 mmol) and Ligase 64 (16.04 mg, 0.036 mmol) in DMF (1 mL) was added HATU (21 mg, 0.054 mmol) followed by DIPEA (24 mg, 0.183 mmol, 0.031 mL) and the reaction mixture was stirred at room temperature for 16 h. The reaction mixture was diluted with water and extracted with EtOAc. The volatiles were removed. The crude residue was purified by prep-HPLC to afford the title compound (1.84 mg, 1.86 μmol, 5% yield) as an off-white solid, trifluoroacetic acid salt. MS (ESI): 967.3 ([M+H]⁺).

### Example 139

### 4-[[4-[9-[4-[4-[3-[3-amino-6-(2-hydroxyphenyl)pyridazin-4-yl]oxy-1-piperidyl]phenyl]piperazin-1-yl]nonyl]-2-pyridyl]methylamino]-2-(2,6-dioxo-3-piperidyl)isoindoline-1,3-dione

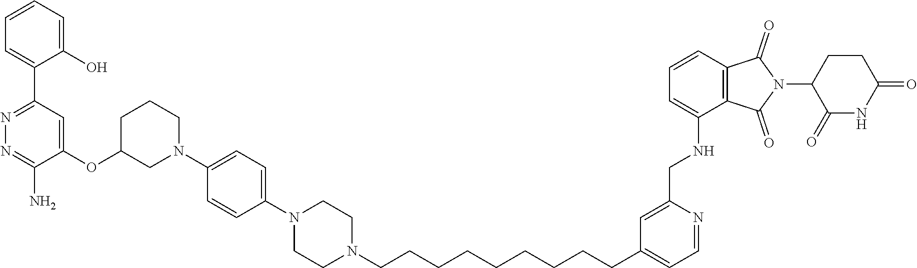 [SMILES: Nc1nnc(-c2ccccc2O)cc1OC1CCCN(c2ccc(N3CCN(CCCCCCCCCc4ccnc(CNc5cccc6c5C(=O)N(C5CCC(=O)NC5=O)C6=O)c4)CC3)cc2)C1]

To a solution of 2-[6-amino-5-[[1-(4-piperazin-1-ylphenyl)-3-piperidyl]oxy]pyridazin-3-yl]phenol (25 mg, 56 μmol) and Ligase 65 (28 mg, 55 μmol) in THF (5 mL) was added dibutyltin dichloride (17 mg, 55 μmol, 12.5 μL) followed by the addition of Trimethyl(phenyl)silane (7 mg, 67 μmol) and the reaction mixture was heated to 80° C. for 16 h. The reaction mixture was cooled to room temperature, diluted with water and extracted with EtOAc. The organic layer was washed with water, brine solution, dried over anhydrous sodium sulfate, filtered and concentrated under reduced pressure. The crude residue was purified by prep-HPLC to afford the title compound (1.3 mg, 1.31 μmol, 2% yield) as an off-white solid, trifluoroacetic acid salt. MS (ESI): 935.3 ([M+H]⁺).

### Example 140

### 2-(2,6-dioxo-3-piperidyl)-4-[[1-[5-oxo-5-[4-[[4-[4-[rac-(3S)-3-[3-amino-6-(2-hydroxyphenyl)pyridazin-4-yl]oxy-1-piperidyl]phenyl]piperazin-1-yl]methyl]-1-piperidyl]pentyl]triazol-4-yl]methoxy]isoindoline-1,3-dione

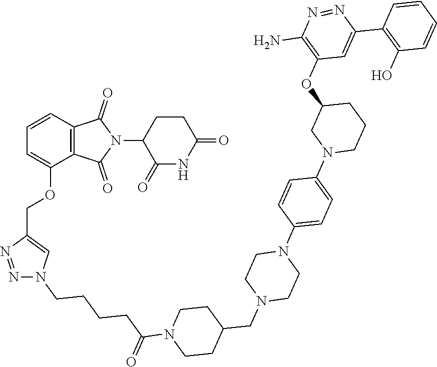 [SMILES: Nc1nnc(-c2ccccc2O)cc1O[C@H]1CCCN(c2ccc(N3CCN(CC4CCN(C(=O)CCCCn5cc(COc6cccc7c6C(=O)N(C6CCC(=O)NC6=O)C7=O)nn5)CC4)CC3)cc2)C1]

To a solution 2-[6-amino-5-[[(3S)-1-[4-[4-(4-piperidylmethyl)piperazin-1-yl]phenyl]-3-piperidyl]oxy]pyridazin-3-yl]phenol (30 mg, 0.055 mmol) and Ligase 63 (25.1 mg, 0.055 mmol) in dimethylformamide (1 mL) was added HATU (25 mg, 0.055 mmol) followed by DIPEA (35 mg, 0.27 mmol, 0.048 mL) and the reaction mixture was stirred at room temperature for 16 h. The reaction mixture was diluted with water and extracted with EtOAc. The volatiles were removed. The crude residue was purified by prep-HPLC to afford the title compound (2.13 mg, 1.99 μmol, 3% yield) an off-white solid, trifluoroacetic acid salt. MS (ESI): 982.3 ([M+H]⁺).

### Example 141

### rac-N-[2-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-2-oxo-ethyl]-1-[2-(2,6-dioxo-3-piperidyl)-1,3-dioxo-isoindolin-5-yl]piperidine-4-carboxamide

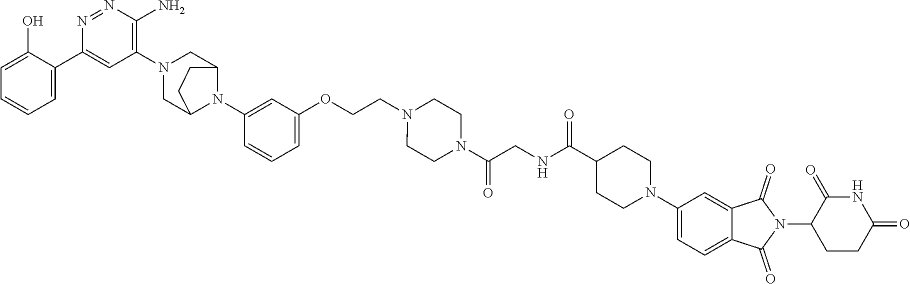 [SMILES: Nc1nnc(-c2ccccc2O)cc1N1CC2CCC(C1)N2c1cccc(OCCN2CCN(C(=O)CNC(=O)C3CCN(c4ccc5c(c4)C(=O)N(C4CCC(=O)NC4=O)C5=O)CC3)CC2)c1]

To a stirred solution of 2-amino-1-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]ethanone trihydrochloride (100 mg, 150 μmol, 1.0 eq), Ligase 15 (57.7 mg, 150 μmol, 1.0 eq) and DIPEA (96.7 mg, 131 μL, 748 μmol, 5.0 eq) in DMF (0.7 mL) at room temperature was added HATU (125 mg, 329 μmol, 2.2 eq). The reaction mixture was stirred for 2 h. The reaction mixture was partitioned between EtOAc/THF (1:1) and saturated aqueous bicarbonate solution. The layers were separated. The aqueous layer was extracted with EtOAc/THF (1:1). The combined organic layers were washed with brine, dried over anhydrous sodium sulfate, and concentrated in vacuo. The crude material was purified on silica column (DCM/MeOH 0-10%) and on amine modified silica column (DCM/MeOH 0-5%) to afford the title compound (32.4 mg, 35 μmol, 23% yield) as a yellow solid. MS (ESI): 464.1 ([M+2H]²⁺).

### Example 142

### rac-5-[4-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]-1-piperidyl]-2-(2,6-dioxo-3-piperidyl)isoindoline-1,3-dione

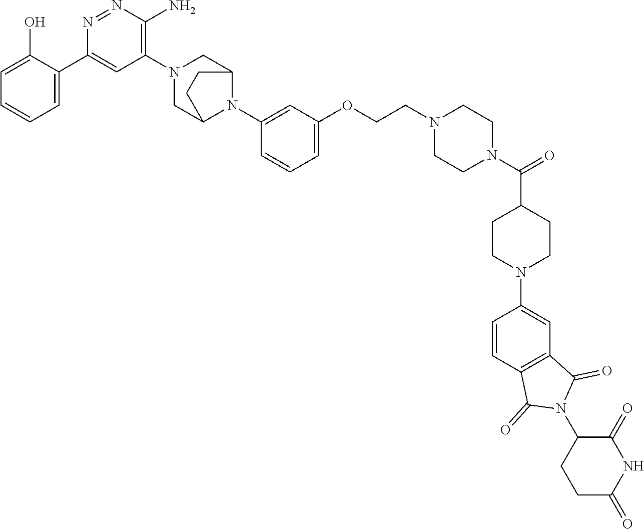 [SMILES: Nc1nnc(-c2ccccc2O)cc1N1CC2CCC(C1)N2c1cccc(OCCN2CCN(C(=O)C3CCN(c4ccc5c(c4)C(=O)N(C4CCC(=O)NC4=O)C5=O)CC3)CC2)c1]

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (25 mg, 49.8 μmol, 1.0 eq), Ligase 15 19.2 mg, 49.8 μmol, 1.0 eq) and DIPEA (19.3 mg, 26.1 μL, 150 μmol, 3.0 eq) in DMF (0.5 mL) at room temperature was added HATU (41.7 mg, 110 μmol, 2.2 eq). The reaction mixture was stirred for 3 h. The purification of the crude material by prep-HPLC to afford the title compound (14.2 mg, 12.9 μmol, 26% yield) as a yellow solid, bis-(2,2,2-trifluoroacetic acid)salt. MS (ESI): 867.6 ([M−H]⁻).

### Example 143

### rac-5-[4-[3-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]propyl]-1-piperidyl]-2-(2,6-dioxo-3-piperidyl)isoindoline-1,3-dione

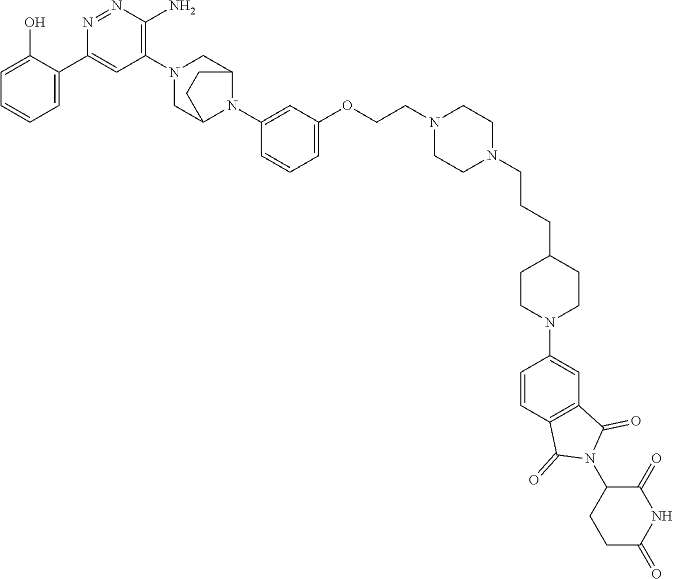 [SMILES: Nc1nnc(-c2ccccc2O)cc1N1CC2CCC(C1)N2c1cccc(OCCN2CCN(CCCC3CCN(c4ccc5c(c4)C(=O)N(C4CCC(=O)NC4=O)C5=O)CC3)CC2)c1]

To a stirred solution of 2-[6-amino-5-[8-[3-(2-piperazin-1-ylethoxy)phenyl]-3,8-diazabicyclo[3.2.1]octan-3-yl]pyridazin-3-yl]phenol (30 mg, 59.8 μmol, 1.0 eq), and Ligase 66 (27.7 mg, 59.8 μmol, 1.0 eq) in DMF (0.5 mL) at room temperature was added sodium iodide (896 μg, 5.98 μmol, 0.1 eq). The reaction mixture was heated at 60° C. for 20 h. The purification of the crude material was done by prep-HPLC to afford the title compound (27.4 mg, 24.7 μmol, 41% yield) as a yellow solid, bis-(2,2,2-trifluoroacetic acid)salt. MS (ESI): 883.4 ([M+H]⁺).

### Example 144

### rac-5-[4-[2-[2-[3-[3-amino-6-(2-hydroxyphenyl)pyridazin-4-yl]-3,8-diazabicyclo[3.2.1]octan-8-yl]phenoxy]ethyl]piperazin-1-yl]-2-(2,6-dioxo-3-piperidyl)isoindoline-1,3-dione

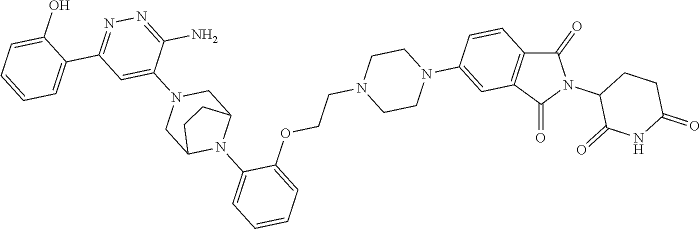 [SMILES: Nc1nnc(-c2ccccc2O)cc1N1CC2CCC(C1)N2c1ccccc1OCCN1CCN(c2ccc3c(c2)C(=O)N(C2CCC(=O)NC2=O)C3=O)CC1]

### a) benzyl 4-[2-(2-bromophenoxy)ethyl]piperazine-1-carboxylate

To a stirred solution of 2-bromophenol (1.44 g, 965 μL, 8.32 mmol, 1.1 eq), benzyl 4-(2-hydroxyethyl)piperazine-1-carboxylate (CAS 14000-67-0, 2 g, 7.57 mmol, 1.0 eq) and triphenylphosphine (2.18 g, 8.32 mmol, 1.1 eq) in THF (25.2 mL) at room temperature was added di-tert-butyl azodicarboxylate (1.92 g, 8.32 mmol, 1.1 eq). The reaction mixture was stirred for 4 h. The reaction mixture was partitioned between EtOAc and water/brine (1:1). The layers were separated. The aqueous layer was extracted with EtOAc. The combined organic layers were washed with brine, dried over anhydrous sodium sulfate, and concentrated in vacuo. The crude material was purified on silica column (heptane/EtOAc 0-60%). 20 ml diethyl ether and 20 ml pentane were added. The solids were removed by filtration. The procedure was repeated with half the volume of the solvent. The crude material was purified on silica column (DCM/MeOH 0-5%) to afford the title compound (2.28 g, 5.44 mmol, 72% yield) as a colourless oil. MS (ESI): 420.9 ([M+H]⁺).

### b) tert-butyl 8-[2-[2-(4-benzyloxycarbonylpiperazin-1-yl)ethoxy]phenyl]-3,8-diazabicyclo[3.2.1]octane-3-carboxylate

A mixture of Pd(OAc)₂ (5.64 mg, 23.8 μmol, 0.2 eq) and 2-dicyclohexylphosphino-2′,6′-diisopropoxybiphenyl (RuPhos) (5.86 mg, 11.9 μmol, 0.1 eq) in toluene (1 mL) at room temperature was degassed, purged with nitrogen, and then stirred at 50° C. for 20 min. In a separate vessel, a stirred suspension of benzyl 4-[2-(2-bromophenoxy)ethyl]piperazine-1-carboxylate (50 mg, 119 μmol, 1.0 eq), tert-butyl 3,8-diazabicyclo[3.2.1]octane-3-carboxylate (25.3 mg, 119 μmol, 1.0 eq) and sodium tert-butoxide (17.2 mg, 179 μmol, 1.5 eq) in toluene (1 mL) at room temperature was degassed, purged with nitrogen, and then stirred at 50° C. The catalyst solution was added and the resulting mixture was stirred at 100° C. overnight. The reaction mixture was partitioned between EtOAc and water/brine (1:1). The layers were separated. The aqueous layer was extracted with EtOAc. The combined organic layers were washed with brine, dried over anhydrous sodium sulfate, and concentrated in vacuo. The crude material was purified on silica column (DCM/MeOH 0-5%) to afford the title compound (33 mg, 59.9 μmol, 50% yield) as a yellow oil. MS (ESI): 551.4 ([M+H]⁺).

### c) benzyl 4-[2-[2-(3,8-diazabicyclo[3.2.1]octan-8-yl)phenoxy]ethyl]piperazine-1-carboxylate

To a stirred solution of tert-butyl 8-[2-[2-(4-benzyloxycarbonylpiperazin-1-yl)ethoxy]phenyl]-3,8-diazabicyclo[3.2.1]octane-3-carboxylate (1.1 g, 2 mmol, 1.0 eq) in 1,4-dioxane (5 mL) at room temperature was added 4 M hydrogen chloride solution in 1,4-dioxane (4.99 mL, 20 mmol, 10.0 eq). The reaction mixture was stirred for 15 h. A light red suspension resulted. The precipitate was collected by filtration, washed with 1,4-dioxane and dried in vacuo to afford the title compound (1.00 g, 1.79 mmol, 89% yield) as a white solid, trihydrochloride salt. MS (ESI): 451.2 ([M+H]⁺).

### d) benzyl 4-[2-[2-[3-(3-amino-6-chloro-pyridazin-4-yl)-3,8-diazabicyclo[3.2.1]octan-8-yl]phenoxy]ethyl]piperazine-1-carboxylate

To a stirred solution of benzyl 4-[2-[2-(3,8-diazabicyclo[3.2.1]octan-8-yl)phenoxy]ethyl]piperazine-1-carboxylate trihydrochloride (300 mg, 536 μmol, 1.0 eq) and K₂CO₃ (370 mg, 2.68 mmol, 5.0 eq) in DMSO (3 mL) at room temperature was added 4-bromo-6-chloropyridazin-3-amine (123 mg, 589 μmol, 1.1 eq). The reaction mixture was stirred at 110° C. for 30 h. The reaction mixture was partitioned between EtOAc/THF (1:2) and water/brine (1:1). The layers were separated. The aqueous layer was extracted with EtOAc/THF (1:1). The combined organic layers were washed with brine, then dried over anhydrous sodium sulfate and concentrated in vacuo. The crude material was purified on silica column (DCM/MeOH 0-10%) to afford the title compound (285.8 mg, 494 μmol, 92% yield) as an orange oil. MS (ESI): 578.3 (³⁵Cl[M+H]⁺).

### e) benzyl 4-[2-[2-[3-[3-amino-6-(2-hydroxyphenyl)pyridazin-4-yl]-3,8-diazabicyclo[3.2.1]octan-8-yl]phenoxy]ethyl]piperazine-1-carboxylate

To a stirred solution of benzyl 4-[2-[2-[3-(3-amino-6-chloro-pyridazin-4-yl)-3,8-diazabicyclo[3.2.1]octan-8-yl]phenoxy]ethyl]piperazine-1-carboxylate (784.3 mg, 1.36 mmol, 1.0 eq) and (2-hydroxyphenyl)boronic acid (468 mg, 3.39 mmol, 2.5 eq) in 1,4-dioxane (35 mL) and water (0.1 mL) at room temperature was added potassium carbonate (656 mg, 4.75 mmol, 3.5 eq). The reaction mixture was degassed with argon for 5 min. RuPhos Pd G3 (113 mg, 136 μmol, 0.1 eq) was added. The reaction mixture was heated at 90° C. for 2 h. The catalyst was removed by filtration and washed with EtOAc and THF. The filtrate was partitioned between EtOAc/THF and water. The layers were separated. The aqueous layer was extracted with EtOAc/THF (1:1). The combined organic layers were washed with brine, dried over anhydrous sodium sulfate, and concentrated in vacuo.

The crude material was purified on silica column (heptane/EtOAc 0-65%) to afford the title compound (287 mg, 451 μmol, 33% yield) as a yellow solid. MS (ESI): 636.4 ([M+H]⁺).

### f) 2-[6-amino-5-[8-[2-(2-piperazin-1-ylethoxy)phenyl]-3,8-diazabicyclo[3.2.1]octan-3-yl]pyridazin-3-yl]phenol

A 100 ml two-necked round-bottomed flask was charged with benzyl 4-[2-[2-[3-[3-amino-6-(2-hydroxyphenyl)pyridazin-4-yl]-3,8-diazabicyclo[3.2.1]octan-8-yl]phenoxy]ethyl]piperazine-1-carboxylate (287 mg, 451 μmol, 1.0 eq), methanol (30 mL) and THF (15 mL). The flask was evacuated to approximately 120 mbar until the solvent began to bubble gently and was then back-filled with argon after 60 s. This procedure was repeated twice. After addition of the catalyst 10% palladium on activated charcoal (48 mg, 45.1 μmol, 0.1 eq) the flask was evacuated to 120 mbar, then back-filled with hydrogen and stirred for 15 h under an atmosphere of ˜1 bar of hydrogen gas. The catalyst was removed by filtration over a Sartorius filter and washed with methanol. The filtrate was concentrated in vacuo to afford the title compound (202 mg, 403 μmol, 89% yield) as a yellow solid. MS (ESI): 502.3 ([M+H]⁺).

### g) rac-5-[4-[2-[2-[3-[3-amino-6-(2-hydroxyphenyl)pyridazin-4-yl]-3,8-diazabicyclo[3.2.1]octan-8-yl]phenoxy]ethyl]piperazin-1-yl]-2-(2,6-dioxo-3-piperidyl)isoindoline-1,3-dione

2-[6-amino-5-[8-[2-(2-piperazin-1-ylethoxy)phenyl]-3,8-diazabicyclo[3.2.1]octan-3-yl]pyridazin-3-yl]phenol (30 mg, 59.8 μmol, 1.0 eq) and 2-(2,6-dioxo-3-piperidyl)-5-fluoro-isoindoline-1,3-dione (CAS 835616-61-0, 16.5 mg, 59.8 μmol, 1.0 eq) were dissolved in DMSO (0.6 mL). The reaction mixture was stirred at 90° C. for 6 h. The reaction mixture was then cooled to room temperature and poured into THF/EtOAc (1:1) and washed sequentially with water and with brine. The organic layer was dried over Na₂SO₄ and concentrated in vacuo. The crude material was purified on silica column (DCM/MeOH 0-5%) to afford the title compound (7 mg, 9.24 μmol, 15% yield) as a yellow solid. MS (ESI): 758.3 ([M+H]⁺).

### Example 145

### 4-[[7-[4-[3-[1-[3-amino-6-(2-hydroxyphenyl)pyridazin-4-yl]azetidin-3-yl]oxybenzoyl]piperazin-1-yl]-7-oxo-heptyl]amino]-2-(2,6-dioxo-3-piperidyl)isoindoline-1,3-dione

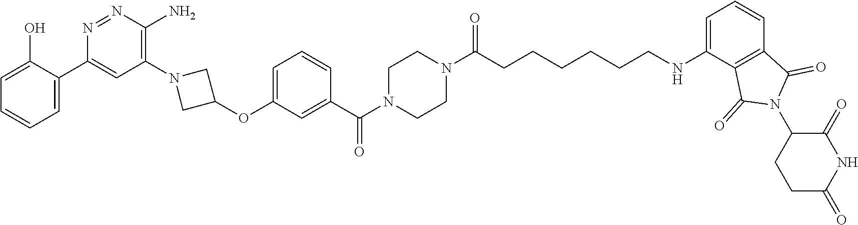 [SMILES: Nc1nnc(-c2ccccc2O)cc1N1CC(Oc2cccc(C(=O)N3CCN(C(=O)CCCCCCNc4cccc5c4C(=O)N(C4CCC(=O)NC4=O)C5=O)CC3)c2)C1]

### a) benzyl 4-(3-hydroxybenzoyl)piperazine-1-carboxylate

To a mixture of 1-Cbz-piperazine (7.97 g, 36.2 mmol, 1.0 eq) and 3-hydroxybenzoic acid (5 g, 36.2 mmol, 1.0 eq) in DCM (50 mL) were added HATU (16.52 g, 43.44 mmol, 1.2 eq) and triethylamine (6.05 mL, 43.4 mmol, 1.2 eq) at 25° C. Then the mixture was stirred at 25° C. for 15 h. The mixture was diluted with water and extracted with DCM. The combined organic layers were dried over anhydrous sodium sulfate, filtered and concentrated in vacuo. The residue was purified on silica column (PE/EtOAc 0-50%) to afford the title compound (8 g, 23.5 mmol, 65% yield) as a white solid. MS (ESI): 341.1 ([M+H]⁺).

### b) benzyl 4-[3-(1-tert-butoxycarbonylazetidin-3-yl)oxybenzoyl]piperazine-1-carboxylate

To a mixture of benzyl 4-(3-hydroxybenzoyl)piperazine-1-carboxylate (8 g, 23.5 mmol, 1.0 eq) and 1-Boc-3-iodoazetidine (8 g, 28.2 mmol, 1.2 eq) in DMF (30 mL) was added cesium carbonate (9.19 g, 28.2 mmol, 1.2 eq) at 25° C. The mixture was stirred at 80° C. for 4 h. To the mixture was added water and it was extracted with ethyl acetate. The organic layers were combined, dried over anhydrous sodium sulfate, filtered and concentrated in vacuo. The residue was purified on silica column (PE/EtOAc 0-50%) to afford the title compound (6 g, 12.11 mmol, 26% yield) as a brown oil. MS (ESI): 496.3 ([M+H]⁺).

### c) benzyl 4-[3-(azetidin-3-yloxy)benzoyl]piperazine-1-carboxylate

To a mixture of benzyl 4-[3-(1-tert-butoxycarbonylazetidin-3-yl)oxybenzoyl]piperazine-1-carboxylate (6.0 g, 6.05 mmol. 1.0 ea) in DCM (20 mL) was added trifluoroacetic acid (10.0 mL, 129.8 mmol, 21 eq) at 25° C. The mixture was stirred at 25° C. for 4 h and then concentrated in vacuo to afford the title compound (4 g, 7.85 mmol, 66% yield) as a brown viscous oil, 2,2,2-trifluoroacetic acid salt. MS (ESI): 396.1 ([M+H]⁺).

### d) benzyl 4-[4-[1-(3-amino-6-chloro-pyridazin-4-yl)azetidin-3-yl]oxybenzoyl]piperazine-1-carboxylate

To a mixture of benzyl 4-[4-(azetidin-3-yloxy)benzoyl]piperazine-1-carboxylate, 2,2,2-trifluoroacetic acid salt (4.0 g, 4 mmol, 1.0 eq) and 4-bromo-6-chloro-pyridazin-3-amine (0.92 g, 4.4 mmol, 1.1 eq) in DMF (50 mL) was added triethylamine (1.4 mL, 10.0 mmol, 2.5 eq) at 25° C. The mixture was stirred at 100° C. for 15 h. The mixture was concentrated in vacuo and purified by prep-HPLC (NH₃) to afford the title compound (800 mg, 1.53 mmol, 38% yield) as a brown solid. MS (ESI): 523.1, 525.1 ([M+H]⁺).

### e) [3-[1-[3-amino-6-(2-hydroxyphenyl)pyridazin-4-yl]azetidin-3-yl]oxyphenyl]-piperazin-1-yl-methanone

To a mixture of benzyl 4-[3-[1-(3-amino-6-chloro-pyridazin-4-yl)azetidin-3-yl]oxybenzoyl]piperazine-1-carboxylate (800 mg, 1.53 mmol, 1.0 eq) and 2-hydroxyphenylboronic acid (422 mg, 3.06 mmol, 2.0 eq) in 1,4-dioxane (15 mL) and water (1.5 mL) were added potassium carbonate (0.4 mL, 4.59 mmol, 3.0 eq) and Ruphos-Pd-G3 (64 mg, 0.080 mmol, 0.05 eq) at 25° C. The mixture was stirred at 100° C. for 15 h. To the mixture was added water and it was extracted with ethyl acetate. The organic layers were combined, dried over anhydrous sodium sulfate, filtered and concentrated in vacuo. The residue was purified by prep-HPLC (NH₃) to afford the title compound (350 mg, 0.780 mmol, 50% yield) as a yellow solid. MS (ESI): 447.1 ([M+H]⁺).

### f) 4-[[7-[4-[3-[1-[3-amino-6-(2-hydroxyphenyl)pyridazin-4-yl]azetidin-3-yl]oxybenzoyl]piperazin-1-yl]-7-oxo-heptyl]amino]-2-(2,6-dioxo-3-piperidyl)isoindoline-1,3-dione

To a mixture of Ligase 1 (32.3 mg, 0.08 mmol, 1.2 eq), Et3N (0.5 mL, 0.2 mmol, 3 eq), [3-[1-[3-amino-6-(2-hydroxyphenyl)pyridazin-4-yl]azetidin-3-yl]oxyphenyl]-piperazin-1-yl-methanone (30.0 mg, 0.07 mmol, 1 eq) in DMF (3 mL) was added T3P (0.3 mL, 0.13 mmol, 2 eq) at 25° C., the reaction mixture was stirred at 25° C. for 15 h. The reaction mixture was purified by prep-HPLC (TFA) to yield the title compound (4 mg, 6% yield) as a yellow solid. MS (ESI): 830.3 ([M+H]⁺).

### Example 146

### 4-[[10-[4-[3-[1-[3-amino-6-(2-hydroxyphenyl)pyridazin-4-yl]azetidin-3-yl]oxybenzoyl]piperazin-1-yl]-10-oxo-decyl]amino]-2-(2,6-dioxo-3-piperidyl)isoindoline-1,3-dione

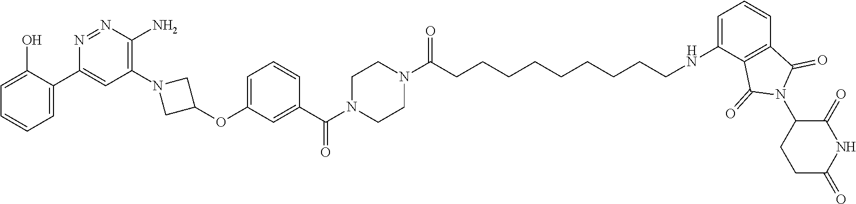 [SMILES: Nc1nnc(-c2ccccc2O)cc1N1CC(Oc2cccc(C(=O)N3CCN(C(=O)CCCCCCCCCNc4cccc5c4C(=O)N(C4CCC(=O)NC4=O)C5=O)CC3)c2)C1]

To a mixture of Ligase 23 (35.7 mg, 0.08 mmol, 1.2 eq), [3-[1-[3-amino-6-(2-hydroxyphenyl)pyridazin-4-yl]azetidin-3-yl]oxyphenyl]-piperazin-1-yl-methanone (30 mg, 0.07 mmol, 1 eq), Et3N (26.0 mg, 0.2 mmol, 3 eq) in DMF (3 mL) was added T3P (0.3 mL, 0.13 mmol, 2 eq) at 25° C., the reaction mixture was stirred at 25° C. for 15 h. The reaction mixture was purified by prep-HPLC (TFA) to yield the title compound (6.2 mg, 0.01 mmol, 10% yield) as a yellow solid. MS (ESI): 872.4 ([M+H]⁺).

### Example 147

### 5-[4-[4-[[3-[3-[3-amino-6-(2-aminophenyl)pyridazin-4-yl]-3,8-diazabicyclo[3.2.1]octan-8-yl]phenyl]methyl]piperazine-1-carbonyl]-1-piperidyl]-2-(2,6-dioxo-3-piperidyl)isoindoline-1,3-dione

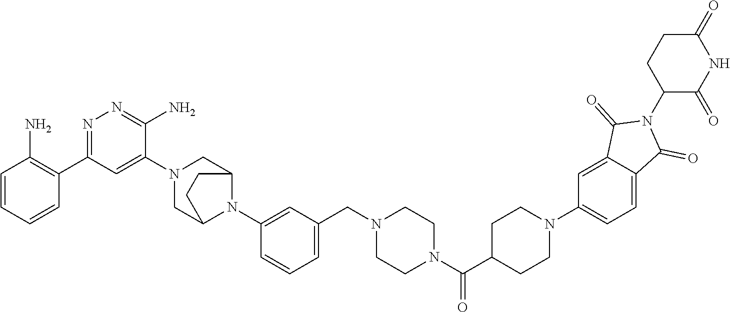 [SMILES: Nc1ccccc1-c1cc(N2CC3CCC(C2)N3c2cccc(CN3CCN(C(=O)C4CCN(c5ccc6c(c5)C(=O)N(C5CCC(=O)NC5=O)C6=O)CC4)CC3)c2)c(N)nn1]

### a) benzyl 4-(3-bromobenzyl)piperazine-1-carboxylate

In a 100 mL round-bottomed flask, 1-bromo-3-(bromomethyl)benzene (6 g, 24 mmol, 1.0 eq) was combined with THF (120 mL). Triethylamine (3.64 g, 5.02 mL, 36 mmol, 1.5 eq) was then added, followed by the addition of benzyl piperazine-1-carboxylate (6.35 g, 5.56 mL, 28.8 mmol, 1.2 eq). The reaction mixture was stirred at room temperature overnight. The solvent was evaporated and the residue was purified on silica column (heptane/EtOAc 0-100%) to afford the title compound (9.26 g, 23.8 mmol, 99% yield) as a colorless oil. MS (ESI): 391.0874 ([M+H]⁺).

### b) tert-butyl 8-(3-((4-((benzyloxy)carbonyl)piperazin-1-yl)methyl)phenyl)-3,8-diazabicyclo[3.2.1]octane-3-carboxylate

Benzyl 4-(3-bromobenzyl)piperazine-1-carboxylate (3 g, 7.71 mmol, 1.0 eq), tert-butyl 3,8-diazabicyclo[3.2.1]octane-3-carboxylate (2.45 g, 11.6 mmol, 1.5 eq) and sodium tert-butoxide (1.11 g, 11.6 mmol, 1.5 eq) were combined with toluene (30 mL). The reaction vessel was degassed by purging with argon. RuPhos Pd G3 95% (173 mg, 771 μmol, 0.1 eq) was added. The reaction mixture was heated to 110° C. and stirred for 16 h. The reaction mixture was filtered through celite. The crude material was purified on silica column (heptane/EtOAc 0-100%) as eluent to afford the title compound (3 g, 5.76 mmol, 74% yield) as a colorless oil. MS (ESI): 391.0874 ([M+H]⁺).

### c) benzyl 4-[[3-(3,8-diazabicyclo[3.2.1]octan-8-yl)phenyl]methyl]piperazine-1-carboxylate

Tert-butyl 8-(3-((4-((benzyloxy)carbonyl)piperazin-1-yl)methyl)phenyl)-3,8-diazabicyclo[3.2.1]octane-3-carboxylate (3 g, 5.76 mmol, 1.0 eq) was dissolved in dioxane (20 mL) and HCl (20 mL, 80 mmol, 13.9 eq) was added. The reaction mixture was stirred at room temperature for 2 h. The mixture was filtered, the solid precipitate was washed with the mother liquor and once with diethyl ether. The precipitate was dried to afford the title compound (2.96 g, 6.48 mmol, 112% yield) as a white solid, hydrochloride salt. MS (ESI): 421.2615 ([M+H]⁺).

### d) benzyl 4-[[3-[3-(3-amino-6-chloro-pyridazin-4-yl)-3,8-diazabicyclo[3.2.1]octan-8-yl]phenyl]methyl]piperazine-1-carboxylate

To a solution of benzyl 4-[[3-(3,8-diazabicyclo[3.2.1]octan-8-yl)phenyl]methyl]piperazine-1-carboxylate (2.95 g, 7.01 mmol, 1.0 eq) and 4-bromo-6-chloropyridazin-3-amine (1.75 g, 8.42 mmol, 1.2 eq) in DMSO (10 mL) was added K₂CO₃ (4.85 g, 35.1 mmol, 5.0 eq). The reaction mixture was stirred for 16 h at 110° C. The reaction mixture was poured into THF/AcOEt 2:1 and washed with water/brine. The organic layer was dried over Na₂SO₄ and concentrated in vacuo. The crude material was purified on silica column (DCM/MeOH 0-10%) to afford the title compound (1.9 g, 3.29 mmol, 46% yield) as a light brown foam. MS (ESI): 548.2542 ([M+H]⁺).

### e) benzyl 4-[[3-[3-[3-amino-6-(2-aminophenyl)pyridazin-4-yl]-3,8-diazabicyclo[3.2.1]octan-8-yl]phenyl]methyl]piperazine-1-carboxylate

To a solution of RuPhos Pd G3 (76.3 mg, 91.2 μmol, 0.1 eq) and benzyl 4-[[3-[3-(3-amino-6-chloro-pyridazin-4-yl)-3,8-diazabicyclo[3.2.1]octan-8-yl]phenyl]methyl]piperazine-1-carboxylate (0.5 g, 912 μmol, 1.0 eq) in dioxane (15 mL) and water (1.5 mL) was added (2-aminophenyl)boronic acid (312 mg, 2.28 mmol, 2.5 eq). The reaction mixture was degassed with argon for 10 min. K₂CO₃ (441 mg, 3.19 mmol, 3.5 eq) was added. The reaction mixture was stirred for 2 h at 90° C. The crude material was purified on silica column (DCM/MeOH 0-10%) to afford the title compound (427 mg, 706 μmol, 77.4% yield) as an off-white solid. MS (ESI): 605.3353 ([M+H]⁺).

### f) 6-(2-aminophenyl)-4-[8-[3-(piperazin-1-ylmethyl)phenyl]-3,8-diazabicyclo[3.2.1]octan-3-yl]pyridazin-3-amine

Benzyl 4-[[3-[3-[3-amino-6-(2-aminophenyl)pyridazin-4-yl]-3,8-diazabicyclo[3.2.1]octan-8-yl]phenyl]methyl]piperazine-1-carboxylate (422 mg, 698 μmol, 1.0 eq) was stirred with Pd—C (74.3 mg, 698 μmol, 1.0 eq) in methanol (12 mL) and THF (2.4 mL) under a hydrogen atmosphere at room temperature overnight. The catalyst was filtered off and the solvent was evaporated under reduced pressure, then dried under high vacuum to afford the title compound (330 mg, 701 μmol, 100% yield) as an off-white foam. MS (ESI): 471.2990 ([M+H]⁺).

### g) 5-[4-[4-[[3-[3-[3-amino-6-(2-aminophenyl)pyridazin-4-yl]-3,8-diazabicyclo[3.2.1]octan-8-yl]phenyl]methyl]piperazine-1-carbonyl]-1-piperidyl]-2-(2,6-dioxo-3-piperidyl)isoindoline-1,3-dione

6-(2-aminophenyl)-4-[8-[3-(piperazin-1-ylmethyl)phenyl]-3,8-diazabicyclo[3.2.1]octan-3-yl]pyridazin-3-amine (40 mg, 85 μmol, 1.0 eq) was combined with Ligase 15 (32.8 mg, 85 μmol, 1.0 eq), HATU (38.8 mg, 102 μmol, 1.2 eq) and DIPEA (33 mg, 44.5 μL, 255 μmol, 3.0 eq) in DMF (500 μL) at room temperature. The reaction was stirred overnight at room temperature. The reaction mixture was submitted to prep-HPLC for purification to afford the title compound (16 mg, 15 μmol, 17.7% yield) as a yellow solid, bis-(2,2,2-trifluoroacetic acid)salt. MS (ESI): 838.4153 ([M+H]⁺).

### Example 148

### rac-3-[4-[1-[2-[4-[[3-[3-[3-Amino-6-(2-hydroxyphenyl)pyridazin-4-yl]-3,8-diazabicyclo[3.2.1]octan-8-yl]phenyl]methyl]piperazin-1-yl]-2-oxo-ethyl]-4-piperidyl]phenoxy]piperidine-2,6-dione

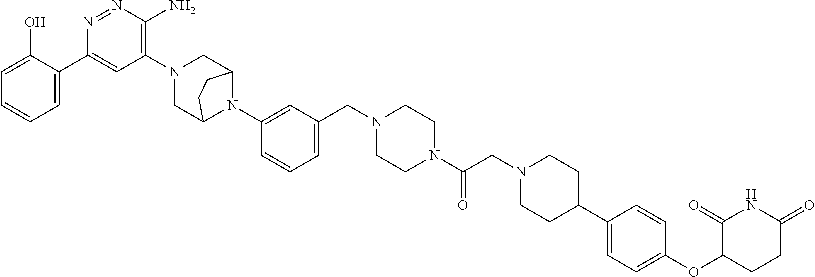 [SMILES: Nc1nnc(-c2ccccc2O)cc1N1CC2CCC(C1)N2c1cccc(CN2CCN(C(=O)CN3CCC(c4ccc(OC5CCC(=O)NC5=O)cc4)CC3)CC2)c1]

2-[6-Amino-5-[8-[3-(piperazin-1-ylmethyl)phenyl]-3,8-diazabicyclo[3.2.1]octan-3-yl]pyridazin-3-yl]phenol; hydrobromide (30 mg, 59 μmol, 1.0 eq; Example 46, Step f) was combined with Ligase 35 (22.6 mg, 59 μmol, 1.0 eq), HATU (44.9 mg, 118 μmol, 2.0 eq) and DIPEA (38.2 mg, 52 μL, 295 μmol, 5.0 eq) in DMF (650 μL). The reaction mixture was stirred at ambient temperature for 8 h and purified by prep-HPLC to afford the title compound (18.2 mg, 0.023 mmol, 32% yield) as a light yellow solid, 2,2,2-trifluoroacetic acid. MS (ESI): 800.4245 ([M+H]⁺).

### Example 149

### rac-3-[4-[1-[2-[4-[[3-[3-[3-Amino-6-(2-hydroxyphenyl)pyridazin-4-yl]-3,8-diazabicyclo[3.2.1]octan-8-yl]phenyl]methyl]piperazin-1-yl]-2-oxo-ethyl]-4-piperidyl]anilino]piperidine-2,6-dione

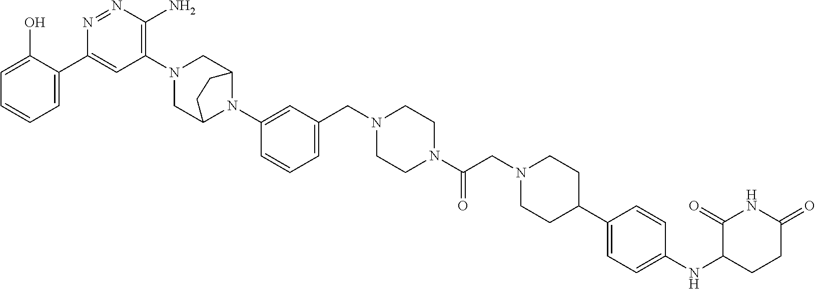 [SMILES: Nc1nnc(-c2ccccc2O)cc1N1CC2CCC(C1)N2c1cccc(CN2CCN(C(=O)CN3CCC(c4ccc(NC5CCC(=O)NC5=O)cc4)CC3)CC2)c1]

2-[6-Amino-5-[8-[3-(piperazin-1-ylmethyl)phenyl]-3,8-diazabicyclo[3.2.1]octan-3-yl]pyridazin-3-yl]phenol; hydrobromide (30 mg, 59 μmol, 1.0 eq; Example 46, Step f) was combined with Ligase 51 (22.5 mg, 59 μmol, 1.0 eq), HATU (44.9 mg, 118 μmol, 2.0 eq) and DIPEA (38.2 mg, 52 μL, 295 μmol, 5.0 eq) in DMF (650 μL). The reaction mixture was stirred at ambient temperature for 8 h and then purified by prep-HPLC to afford the title compound (24 mg, 0.030 mmol, 44% yield) as a light yellow solid, 2,2,2-trifluoroacetic acid salt. MS (ESI): 799.8 ([M+H]⁺).

### Example 150

### 4-[[10-[4-[4-[1-[3-amino-6-(2-hydroxyphenyl)pyridazin-4-yl]azetidin-3-yl]oxybenzoyl]piperazin-1-yl]-10-oxo-decyl]amino]-2-(2,6-dioxo-3-piperidyl)isoindoline-1,3-dione

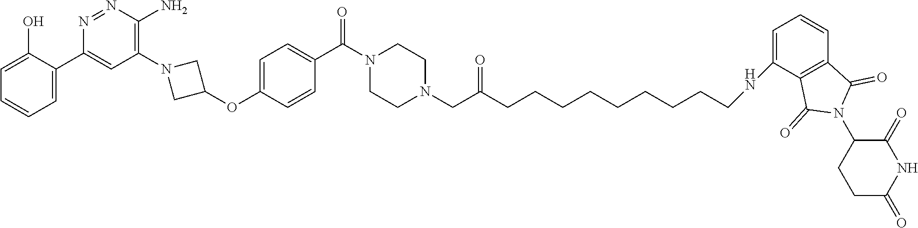 [SMILES: Nc1nnc(-c2ccccc2O)cc1N1CC(Oc2ccc(C(=O)N3CCN(CC(=O)CCCCCCCCCNc4cccc5c4C(=O)N(C4CCC(=O)NC4=O)C5=O)CC3)cc2)C1]

### a) benzyl 4-(4-hydroxybenzoyl)piperazine-1-carboxylate

To a solution of 4-hydroxybenzoic acid (10 g, 72.4 mmol, 1.0 eq) in DMF (140 mL) were added 1-Cbz-piperazine (16 g, 72.4 mmol, 1.0 eq), HATU (31 g, 79.64 mmol, 1.1 eq) and triethylamine (20 mL, 144.8 mmol, 2.0 eq), and then the reaction was stirred at 25° C. for 2 h. The mixture was diluted with water (120 mL) and extracted with EtOAc. The combined organic phase was washed with brine, dried over sodium sulfate, filtered and concentrated under reduced pressure. The crude residue was purified on silica column (heptane/EtOAc 25-50%) to afford the title compound (18 g, 52 mmol, 73% yield) as a brown liquid. MS (ESI): 341.1 ([M+H]⁺).

### b) benzyl 4-[4-(1-tert-butoxycarbonylazetidin-3-yl)oxybenzoyl]piperazine-1-carboxylate

To a solution of benzyl 4-(4-hydroxybenzoyl)piperazine-1-carboxylate (12 g, 35.2 mmol, 1.0 eq) in DMF (140 mL) was added 1-Boc-3-iodoazetidine (12 g, 42.3 mmol, 1.2 eq) and cesium carbonate (14 g, 42.3 mmol, 1.2 eq) and the reaction was stirred at 80° C. for 12 h. The residue was diluted with water and extracted with EtOAc. The combined organic layer was dried over anhydrous sodium sulfate, filtered and concentrated in vacuo. The crude residue was purified on silica column (heptane/EtOAc 25-50%) to afford the title compound (10 g, 20.1 mmol, 57% yield) as a yellow liquid. MS (ESI): 440.1 ([M-56+H]⁺).

### c) benzyl 4-[4-(azetidin-3-yloxy)benzoyl]piperazine-1-carboxylate

To a solution of benzyl 4-[4-(1-tert-butoxycarbonylazetidin-3-yl)oxybenzoyl]piperazine-1-carboxylate (10 g, 20.1 mmol, 1.0 eq) in DCM (50 mL) was added trifluoroacetic acid (50.0 mL, 648 mmol, 32 eq) and the reaction was stirred at 25° C. for 1 h. The reaction solution was concentrated to afford the title compound (7 g, 17.7 mmol, 87% yield) as a yellow liquid, trifluoroacetic acid salt. MS (ESI): 396.1 ([M+H]⁺).

### d) benzyl 4-[4-[1-(3-amino-6-chloro-pyridazin-4-yl)azetidin-3-yl]oxybenzoyl]piperazine-1-carboxylate

To a solution of benzyl 4-[4-(azetidin-3-yloxy)benzoyl]piperazine-1-carboxylate, TFA salt (7.0 g, 17.7 mmol, 1.0 eq) in DMF (70 mL) was added 4-bromo-6-chloro-pyridazin-3-amine (3.7 g, 17.7 mmol, 1.0 eq) and triethylamine (10 mL, 70.8 mmol, 4.0 eq) and the reaction was stirred at 100° C. for 12 h. The mixture was diluted with water and extracted with EtOAc. The combined organic phase was washed with brine, dried over sodium sulfate, filtered and concentrated under reduced pressure. The crude residue was purified on silica column (heptane/EtOAc 67-100%) to afford the title compound (4 g, 7.6 mmol, 43% yield) as a brown gum. MS (ESI): 523.2 ([M+H]⁺).

### e) [4-[1-[3-amino-6-(2-hydroxyphenyl)pyridazin-4-yl]azetidin-3-yl]oxyphenyl]-piperazin-1-yl-methanone

A solution of 2-hydroxyphenylboronic acid (263 mg, 1.91 mmol, 2.0 eq), 4-[4-[1-(3-amino-6-chloro-pyridazin-4-yl)azetidin-3-yl]oxybenzoyl]piperazine-1-carboxylate (500 mg, 0.960 mmol, 1.0 eq), Ruphos-Pd-G3 (75 mg, 0.050 mmol, 0.05 eq) and K₂CO₃ (400 mg, 2.87 mmol, 3.0 eq) in 1,4-dioxane (5 mL) and water (0.5 mL) was stirred at 90° C. for 2 h. The reaction solution was filtered and was then submitted to prep-HPLC for purification to afford the title compound (60 mg, 0.130 mmol, 14% yield) as a yellow solid. MS (ESI): 447.2 ([M+H]⁺).

### f) 4-[[10-[4-[4-[1-[3-amino-6-(2-hydroxyphenyl)pyridazin-4-yl]azetidin-3-yl]oxybenzoyl]piperazin-1-yl]-10-oxo-decyl]amino]-2-(2,6-dioxo-3-piperidyl)isoindoline-1,3-dione

A mixture of [4-[1-[3-amino-6-(2-hydroxyphenyl)pyridazin-4-yl]azetidin-3-yl]oxyphenyl]-piperazin-1-yl-methanone (25.0 mg, 0.06 mmol, 1 eq), Ligase 23 (29.8 mg, 0.07 mmol, 1.2 eq), Et3N (21.7 mg, 0.17 mmol, 3 eq) in DMF (3 mL) was added T3P (0.3 mL, 0.110 mmol, 2 eq) at 25° C., the reaction mixture was stirred at 25° C. for 1 h. The reaction mixture was purified by prep-HPLC (TFA) to yield the title compound (2.1 mg, 4% yield) as a yellow solid. MS (ESI): 872.5 ([M+H]⁺).

### Example 151

### N-[1-[3-amino-6-(2-hydroxyphenyl)pyridazin-4-yl]-4-phenyl-4-piperidyl]-4-[4-[4-[(2,6-dioxo-3-piperidyl)oxy]phenyl]-1-piperidyl]butanamide

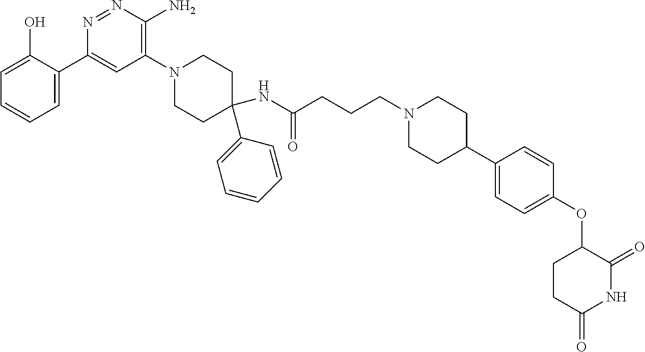 [SMILES: Nc1nnc(-c2ccccc2O)cc1N1CCC(NC(=O)CCCN2CCC(c3ccc(OC4CCC(=O)NC4=O)cc3)CC2)(c2ccccc2)CC1]

2-(6-amino-5-(4-amino-4-phenylpiperidin-1-yl)pyridazin-3-yl)phenol (40 mg, 111 μmol, 1.0 eq) was combined with Ligase 39 (50 mg, 122 μmol, 1.1 eq), HATU (84.2 mg, 221 μmol, 2.0 ea) and DIPEA (71.5 mg, 96.6 μL, 553 μmol, 5.0 eq) in DMF (400 μL). The brown solution was stirred at room temperature for 2 h and was then submitted to prep-HPLC for purification to afford the title compound (7 mg, 7.4 μmol, 6% yield) as a white solid, bis-(2,2,2-trifluoroacetic acid)salt. MS (ESI): 718.3715 ([M+H]⁺).

### Example 152

### rac-5-[4-[4-[3-[1-[3-amino-6-(2-hydroxyphenyl)pyridazin-4-yl]azetidin-3-yl]oxyphenyl]piperazine-1-carbonyl]-1-piperidyl]-2-(2,6-dioxo-3-piperidyl)isoindoline-1,3-dione

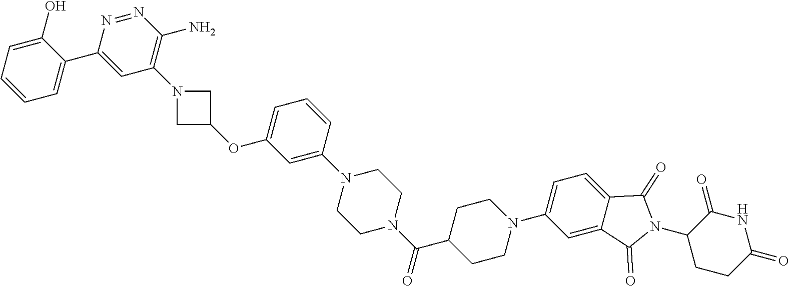 [SMILES: Nc1nnc(-c2ccccc2O)cc1N1CC(Oc2cccc(N3CCN(C(=O)C4CCN(c5ccc6c(c5)C(=O)N(C5CCC(=O)NC5=O)C6=O)CC4)CC3)c2)C1]

To a stirred solution of 2-[6-amino-5-[3-(3-piperazin-1-ylphenoxy)azetidin-1-yl]pyridazin-3-yl]phenol (30 mg, 71.7 μmol, 1.0 eq), Ligase 15 (30.4 mg, 78.9 μmol, 1.1 eq) and DIPEA (46.3 mg, 62.6 μL, 358 μmol, 5.0 eq) in DMSO (0.5 mL) at room temperature was added HATU (54.5 mg, 143 μmol, 2.0 eq). The reaction mixture was stirred at room temperature for 2 h. The crude material was purified by prep-HPLC to afford the title compound (8 mg, 7.8 μmol, 11% yield) as a yellow solid, bis-(2,2,2-trifluoroacetic acid)salt. MS (ESI): 784.3 ([M−H]⁻).

### Example 153

### rac-5-[4-[3-[4-[3-[1-[3-amino-6-(2-hydroxyphenyl)pyridazin-4-yl]azetidin-3-yl]oxyphenyl]piperazin-1-yl]-3-oxo-propyl]-1-piperidyl]-2-(2,6-dioxo-3-piperidyl)isoindoline-1,3-dione

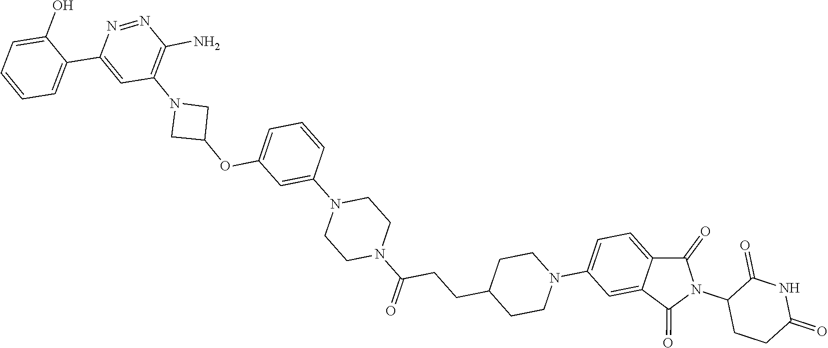 [SMILES: Nc1nnc(-c2ccccc2O)cc1N1CC(Oc2cccc(N3CCN(C(=O)CCC4CCN(c5ccc6c(c5)C(=O)N(C5CCC(=O)NC5=O)C6=O)CC4)CC3)c2)C1]

To a stirred solution of 2-[6-amino-5-[3-(3-piperazin-1-ylphenoxy)azetidin-1-yl]pyridazin-3-yl]phenol (30 mg, 71.7 μmol, 1.0 eq), Ligase 12 (32.6 mg, 78.9 μmol, 1.1 eq) and DIPEA (46.3 mg, 62.6 μL, 358 μmol, 5.0 eq) in DMSO (0.5 mL) at room temperature was added HATU (54.5 mg, 143 μmol, 2.0 eq). The reaction mixture was stirred at room temperature for 2 h. The crude material was purified by prep-HPLC to afford the title compound (15 mg, 14.4 μmol, 20% yield) as a yellow solid, bis-(2,2,2-trifluoroacetic acid)salt. MS (ESI): 812.4 ([M−H]⁻).

### Example 154

### rac-5-[4-[3-[4-[[3-[1-[3-amino-6-(2-hydroxyphenyl)pyridazin-4-yl]azetidin-3-yl]oxyphenyl]methyl]piperazin-1-yl]-3-oxo-propyl]-1-piperidyl]-2-(2,6-dioxo-3-piperidyl)isoindoline-1,3-dione

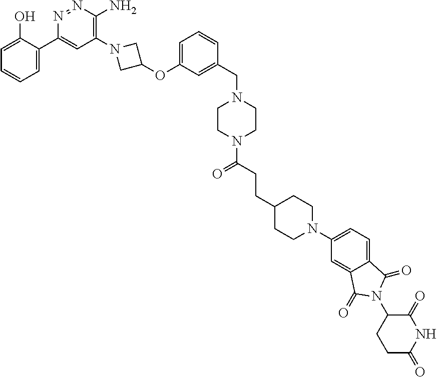 [SMILES: Nc1nnc(-c2ccccc2O)cc1N1CC(Oc2cccc(CN3CCN(C(=O)CCC4CCN(c5ccc6c(c5)C(=O)N(C5CCC(=O)NC5=O)C6=O)CC4)CC3)c2)C1]

To a stirred solution of 2-[6-amino-5-[3-[3-(piperazin-1-ylmethyl)phenoxy]azetidin-1-yl]pyridazin-3-yl]phenol (30 mg, 69.4 μmol, 1.0 eq), Ligase 12 (28.7 mg, 69.4 μmol, 1.0 eq) and HATU (52.7 mg, 139 μmol, 2.0 eq) in DMSO (0.8 mL) at room temperature was added DIPEA (26.9 mg, 36.3 μL, 208 μmol, 3.0 eq). The reaction mixture was stirred for 3 h and purified by prep-HPLC to afford the title compound (25.4 mg, 24.1 μmol, 35% yield) as a yellow solid, bis-(2,2,2-trifluoroacetic acid)salt. MS (ESI): 826.4 ([M−H]⁻).

### Example 155

### rac-5-[4-[4-[[3-[1-[3-amino-6-(2-hydroxyphenyl)pyridazin-4-yl]azetidin-3-yl]oxyphenyl]methyl]piperazine-1-carbonyl]-1-piperidyl]-2-(2,6-dioxo-3-piperidyl)isoindoline-1,3-dione

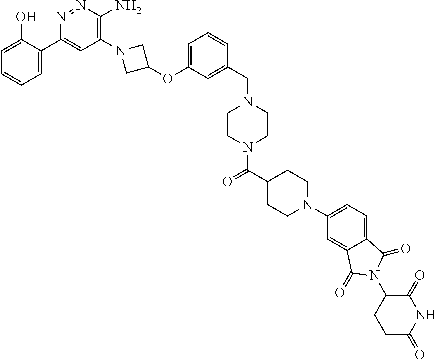 [SMILES: Nc1nnc(-c2ccccc2O)cc1N1CC(Oc2cccc(CN3CCN(C(=O)C4CCN(c5ccc6c(c5)C(=O)N(C5CCC(=O)NC5=O)C6=O)CC4)CC3)c2)C1]

To a stirred solution of 2-[6-amino-5-[3-[3-(piperazin-1-ylmethyl)phenoxy]azetidin-1-yl]pyridazin-3-yl]phenol (30 mg, 69.4 μmol, 1.0 eq), Ligase 15 (30 mg, 77.8 μmol, 1.12 eq) and HATU (52.7 mg, 139 μmol, 2.0 eq) in DMSO (0.8 mL) at room temperature was added DIPEA (26.9 mg, 36.3 μL, 208 μmol, 3.0 eq). The reaction mixture was stirred for 3 h and purified by prep-HPLC to afford the title compound (24.7 mg, 24 μmol, 35% yield) as a yellow solid, bis-(2,2,2-trifluoroacetic acid)salt. MS (ESI): 798.3 ([M−H]⁻).

### Example 156

### rac-3-[4-[1-[2-[4-[[3-[1-[3-amino-6-(2-hydroxyphenyl)pyridazin-4-yl]azetidin-3-yl]oxyphenyl]methyl]piperazin-1-yl]-2-oxo-ethyl]-4-piperidyl]anilino]piperidine-2,6-dione

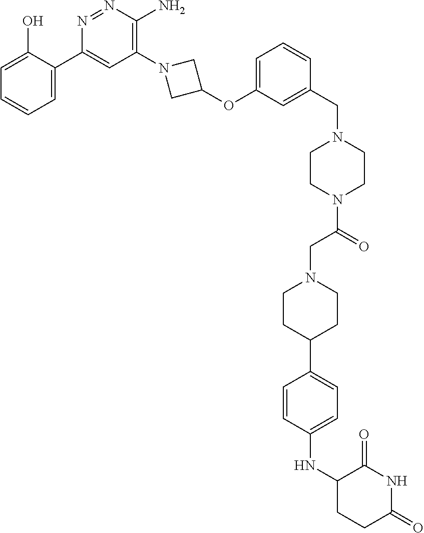 [SMILES: Nc1nnc(-c2ccccc2O)cc1N1CC(Oc2cccc(CN3CCN(C(=O)CN4CCC(c5ccc(NC6CCC(=O)NC6=O)cc5)CC4)CC3)c2)C1]

To a stirred solution of 2-[6-amino-5-[3-[3-(piperazin-1-ylmethyl)phenoxy]azetidin-1-yl]pyridazin-3-yl]phenol (30 mg, 69.4 μmol, 1.0 eq), Ligase 51 (26.5 mg, 69.4 μmol, 1.0 eq) and HATU (52.7 mg, 139 μmol, 2.0 eq) in DMSO (0.8 mL) at room temperature was added DIPEA (26.9 mg, 36.3 μL, 208 μmol, 3.0 eq). The reaction mixture was stirred for 3 h and purified by prep-HPLC to afford the title compound (14.8 mg, 15 μmol, 22% yield) as an off-white solid, bis-(2,2,2-trifluoroacetic acid)salt. MS (ESI): 758.4 ([M−H]⁻).

### Example 157

### rac-2-(2,6-dioxo-3-piperidyl)-5-[rel-(3aS,6aR)-5-[4-[2-[3-[3-[3-amino-6-(2-hydroxyphenyl)pyridazin-4-yl]-3,8-diazabicyclo[3.2.1]octan-8-yl]phenoxy]ethyl]piperazine-1-carbonyl]-1,3,3a,4,6,6a-hexahydropyrrolo[3,4-c]pyrrol-2-yl]isoindoline-1,3-dione

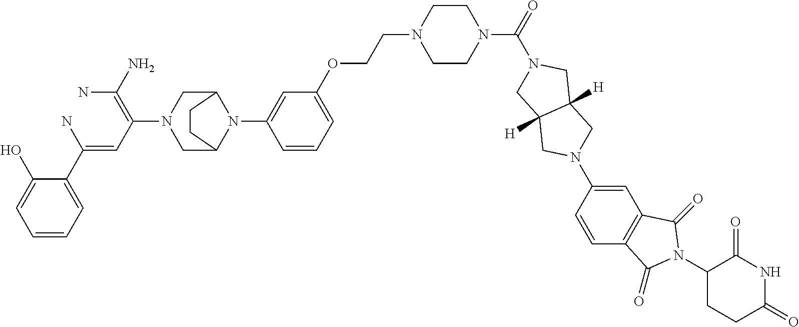 [SMILES: NC(N)=C(/C=C(\N)c1ccccc1O)N1CC2CCC(C1)N2c1cccc(OCCN2CCN(C(=O)N3C[C@@H]4CN(c5ccc6c(c5)C(=O)N(C5CCC(=O)NC5=O)C6=O)C[C@@H]4C3)CC2)c1]

To a stirred solution of (4-nitrophenyl) 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate (35 mg, 52.5 μmol, 1.0 eq) and Ligase 67 (23.4 mg, 57.7 μmol, 1.1 eq) in DMSO (0.6 mL) at room temperature was added DIPEA (17 mg, 22.9 μL, 131 μmol, 2.5 eq). The reaction mixture was stirred at 115° C. for 20 h. The crude material was purified on silica column (DCM/MeOH 0-5%) followed by prep-HPLC to afford the title compound (7 mg, 6.2 μmol, 10% yield) as a yellow solid, bis-(2,2,2-trifluoroacetic acid)salt. MS (ESI): 896.4 ([M+H]⁺).

### Example 158

### 5-[4-[4-[[3-[3-[3-amino-6-(5-fluoro-2-hydroxy-phenyl)pyridazin-4-yl]-3,8-diazabicyclo[3.2.1]octan-8-yl]phenyl]methyl]piperazine-1-carbonyl]-1-piperidyl]-2-(2,6-dioxo-3-piperidyl)isoindoline-1,3-dione

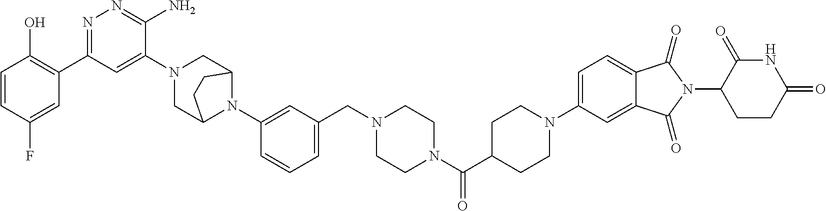 [SMILES: Nc1nnc(-c2cc(F)ccc2O)cc1N1CC2CCC(C1)N2c1cccc(CN2CCN(C(=O)C3CCN(c4ccc5c(c4)C(=O)N(C4CCC(=O)NC4=O)C5=O)CC3)CC2)c1]

### a) benzyl 4-(3-bromobenzyl)piperazine-1-carboxylate

1-bromo-3-(bromomethyl)benzene (6 g, 24 mmol, 1.0 eq) was combined with THF (120 mL). Triethylamine (3.64 g, 5.02 mL, 36 mmol, 1.5 eq) was then added, followed by the addition of benzyl piperazine-1-carboxylate (6.35 g, 5.56 mL, 28.8 mmol, 1.2 eq). The reaction mixture was stirred at room temperature overnight. The solvent was evaporated and the residue was purified on silica column (heptane/EtOAc 0-100%) to afford the title compound (9.26 g, 23.8 mmol, 99% yield) as a colorless oil. MS (ESI): 391.0874 ([M+H]⁺).

### b) tert-butyl 8-(3-((4-((benzyloxy)carbonyl)piperazin-1-yl)methyl)phenyl)-3,8-diazabicyclo[3.2.1]octane-3-carboxylate

Benzyl 4-(3-bromobenzyl)piperazine-1-carboxylate (3 g, 7.71 mmol, 1.0 eq), tert-butyl 3,8-diazabicyclo[3.2.1]octane-3-carboxylate (2.45 g, 11.6 mmol, 1.5 eq) and sodium tert-butoxide (1.11 g, 11.6 mmol, 1.5 eq) were combined with toluene (30 mL). The reaction vessel was degassed by purging with argon. RuPhos Pd G3 95% (173 mg, 771 μmol, 0.1 eq) was added. The reaction mixture was heated to 110° C. and stirred for 16 h. The reaction mixture was filtered through celite. The crude material was purified on silica column (heptane/EtOAc 0-100%) to afford the title compound (3 g, 5.76 mmol, 74% yield) as a colorless oil. MS (ESI): 391.0874 ([M+H]⁺).

### c) benzyl 4-[[3-(3,8-diazabicyclo[3.2.1]octan-8-yl)phenyl]methyl]piperazine-1-carboxylate

Tert-butyl 8-(3-((4-((benzyloxy)carbonyl)piperazin-1-yl)methyl)phenyl)-3,8-diazabicyclo[3.2.1]octane-3-carboxylate (3 g, 5.76 mmol, 1.0 eq) was dissolved in dioxane (20 mL) and HCl (20 mL, 80 mmol, 13.9 eq) was added. The reaction mixture was stirred at room temperature for 2 h. The mixture was filtered, the solid precipitate was washed with the mother liquor and once with diethyl ether. The precipitate was dried to afford the title compound (2.96 g, 6.48 mmol, 112% yield) as a white solid, hydrochloride salt. MS (ESI): 421.2615 ([M+H]⁺).

### d) benzyl 4-[[3-[3-(3-amino-6-chloro-pyridazin-4-yl)-3,8-diazabicyclo[3.2.1]octan-8-yl]phenyl]methyl]piperazine-1-carboxylate

To a solution of benzyl 4-[[3-(3,8-diazabicyclo[3.2.1]octan-8-yl)phenyl]methyl]piperazine-1-carboxylate (2.95 g, 7.01 mmol, 1.0 eq) and 4-bromo-6-chloropyridazin-3-amine (1.75 g, 8.42 mmol, 1.2 eq) in DMSO (10 mL) was added K₂CO₃ (4.85 g, 35.1 mmol, 5.0 eq). The reaction mixture was stirred for 16 h at 110° C. The reaction mixture was poured into THF/AcOEt 2:1 and washed with water/brine. The organic layer was dried over Na₂SO₄ and concentrated in vacuo. The crude material was purified on silica column (DCM/MeOH 0-10%) to afford the title compound (1.9 g, 3.29 mmol, 46% yield) as a light brown foam. MS (ESI): 548.2542 ([M+H]⁺).

### e) benzyl 4-[[3-[3-[3-amino-6-(5-fluoro-2-hydroxy-phenyl)pyridazin-4-yl]-3,8-diazabicyclo[3.2.1]octan-8-yl]phenyl]methyl]piperazine-1-carboxylate

Benzyl 4-[[3-[3-(3-amino-6-chloro-pyridazin-4-yl)-3,8-diazabicyclo[3.2.1]octan-8-yl]phenyl]methyl]piperazine-1-carboxylate (1 g, 1.82 mmol, 1.0 eq) was stirred with (5-fluoro-2-hydroxyphenyl)boronic acid (284 mg, 1.82 mmol, 1.0 eq), potassium carbonate (883 mg, 6.39 mmol, 3.5 eq) and RuPhos Pd G3 (153 mg, 182 μmol, 0.1 eq) in dioxane (10 mL) and water (1 mL) at 120° C. in a sealed tube for 2 h. The crude material was purified on silica column (heptane/EtOAc 50-100%)) and (EtOAc/MeOH 0-10%) to afford the title compound (827 mg, 1.33 mmol, 72% yield) as a yellow solid. MS (ESI): 624.309 ([M+H]⁺).

### f) 2-[6-amino-5-[8-[3-(piperazin-1-ylmethyl)phenyl]-3,8-diazabicyclo[3.2.1]octan-3-yl]pyridazin-3-yl]-4-fluoro-phenol

Benzyl 4-[[3-[3-[3-amino-6-(5-fluoro-2-hydroxy-phenyl)pyridazin-4-yl]-3,8-diazabicyclo[3.2.1]octan-8-yl]phenyl]methyl]piperazine-1-carboxylate (820 mg, 1.31 mmol, 1.0 eq) was suspended in methanol (20 mL) and THF (4 mL). Pd—C (140 mg, 131 μmol, 0.1 eq) was added, the reaction was degassed and purged with argon and then hydrogen three times and was stirred under hydrogen atmosphere overnight. The reaction mixture was filtered over decalite, washed with DCM/methanol (9:1) and evaporated to afford the title compound (323 mg, 485 μmol, 36% yield) as a light brown solid. MS (ESI): 490.2712 ([M+H]⁺).

### g) 5-[4-[4-[[3-[3-[3-amino-6-(5-fluoro-2-hydroxy-phenyl)pyridazin-4-yl]-3,8-diazabicyclo[3.2.1]octan-8-yl]phenyl]methyl]piperazine-1-carbonyl]-1-piperidyl]-2-(2,6-dioxo-3-piperidyl)isoindoline-1,3-dione

2-[6-amino-5-[8-[3-(piperazin-1-ylmethyl)phenyl]-3,8-diazabicyclo[3.2.1]octan-3-yl]pyridazin-3-yl]-4-fluoro-phenol (50 mg, 102 μmol, 1.0 eq) was combined with Ligase 15 (39.4 mg, 102 μmol, 1.0 eq), HATU (48.5 mg, 128 μmol, 1.25 eq) and DIPEA (39.6 mg, 53.5 μL, 306 μmol, 3.0 eq) in DMF (500 μL). The reaction was stirred at room temperature for 2 h. The reaction mixture was purified by prep-HPLC to afford the title compound (12.4 mg, 9.14 μmol, 8% yield) as a yellow solid, bis-(2,2,2-trifluoroacetic acid)salt. MS (ESI): 857.3899 ([M+H]⁺).

### Example 159

### 5-(4-((6-(1-(3-amino-6-(2-hydroxyphenyl)pyridazin-4-yl)-4-phenylpiperidine-4-carbonyl)-2,6-diazaspiro[3.3]heptan-2-yl)methyl)piperidin-1-yl)-2-(2,6-dioxopiperidin-3-yl)isoindoline-1,3-dione

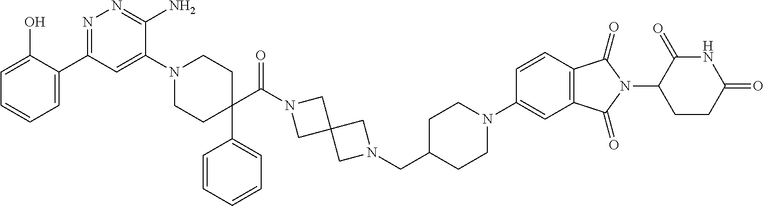 [SMILES: Nc1nnc(-c2ccccc2O)cc1N1CCC(C(=O)N2CC3(CN(CC4CCN(c5ccc6c(c5)C(=O)N(C5CCC(=O)NC5=O)C6=O)CC4)C3)C2)(c2ccccc2)CC1]

(1-(3-amino-6-(2-hydroxyphenyl)pyridazin-4-yl)-4-phenylpiperidin-4-yl)(2,6-diazaspiro[3.3]heptan-2-yl)methanone (17 mg, 36.1 μmol, 1.0 eq) and Ligase 33 (18.8 mg, 43.4 μmol, 1.2 eq) were dissolved in DMSO (400 μL). Potassium iodide (600 μg, 3.61 μmol, 0.1 eq) was added and the mixture was stirred at room temperature for 16 h. The reaction mixture was purified directly by prep-HPLC to afford the title compound (3.8 mg, 3.71 μmol, 10% yield) as a yellow solid, formate salt. MS (ESI): 824.7 ([M+H]⁺).

### Example 160

### 5-[4-[4-[2-[3-[8-[3-amino-6-(5-fluoro-2-hydroxy-phenyl)pyridazin-4-yl]-3,8-diazabicyclo[3.2.1]octan-3-yl]phenoxy]ethyl]piperazine-1-carbonyl]-1-piperidyl]-2-(2,6-dioxo-3-piperidyl)isoindoline-1,3-dione

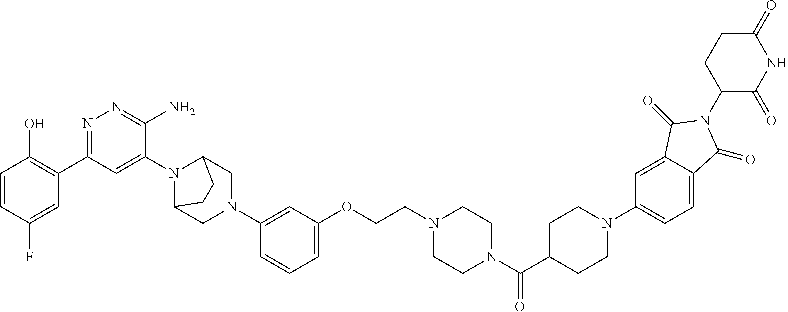 [SMILES: Nc1nnc(-c2cc(F)ccc2O)cc1N1C2CCC1CN(c1cccc(OCCN3CCN(C(=O)C4CCN(c5ccc6c(c5)C(=O)N(C5CCC(=O)NC5=O)C6=O)CC4)CC3)c1)C2]

2-[6-amino-5-[3-[3-(2-piperazin-1-ylethoxy)phenyl]-3,8-diazabicyclo[3.2.1]octan-8-yl]pyridazin-3-yl]-4-fluoro-phenol (40 mg, 77 μmol, 1.0 eq) was combined with Ligase 15 (29.7 mg, 77 μmol, 1.0 eq), HATU (35.1 mg, 92.4 μmol, 1.2 eq) and DIPEA (29.8 mg, 40.3 μL, 231 μmol, 3.0 eq) in DMF (500 μL) at room temperature. The reaction was stirred overnight at room temperature. The reaction mixture was purified by prep-HPLC to afford the title compound (42 mg, 36.2 μmol, 47% yield) as a yellow solid, bis-(2,2,2-trifluoroacetic acid)salt. MS (ESI): 887.4002 ([M+H]⁺).

### Example 161

### rac-5-[4-[3-[4-[2-[2-[3-[3-Amino-6-(2-hydroxyphenyl)pyridazin-4-yl]-3,8-diazabicyclo[3.2.1]octan-8-yl]pyrimidin-4-yl]oxyethyl]piperazin-1-yl]propoxy]-1-piperidyl]-2-(2,6-dioxo-3-piperidyl)isoindoline-1,3-dione

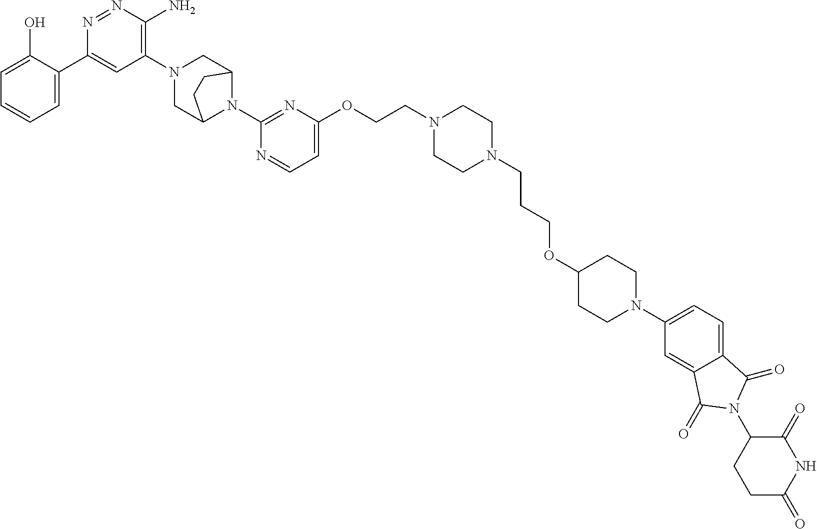 [SMILES: Nc1nnc(-c2ccccc2O)cc1N1CC2CCC(C1)N2c1nccc(OCCN2CCN(CCCOC3CCN(c4ccc5c(c4)C(=O)N(C4CCC(=O)NC4=O)C5=O)CC3)CC2)n1]

### a) Benzyl 4-[2-(2-chloropyrimidin-4-yl)oxyethyl]piperazine-1-carboxylate

To a solution of benzyl 4-(2-hydroxyethyl)piperazine-1-carboxylate (0.89 g, 3.36 mmol, 1.0 eq) in DMA (10 mL) was added portionwise sodium hydride (0.2 g, 5.0 mmol, 1.5 eq) and the reaction mixture was stirred at ambient temperature for 0.5 h. Then a solution of 2,4-dichloropyrimidine (0.50 g, 3.36 mmol, 1.0 eq) in DMA (5 mL) was added dropwise and the reaction mixture was stirred at ambient temperature for 8 h. Another portion of sodium hydride (0.2 g, 5.0 mmol, 1.5 eq) was added and stirring at ambient temperature continued for an additional 2 h. The reaction mixture was poured on ice-water, a saturated NH₄Cl solution was added and the mixture extracted with EtOAc. The combined organic layers were washed with brine, dried over MgSO₄ and concentrated in vacuo. The crude material was purified on silica column (heptane/EtOAc 0-100%) to afford the title compound (0.41 g, 1.09 mmol, 32% yield) as a colorless oil. MS (ESI): 377.3 ([M+H]⁺).

### b) tert-Butyl 8-[4-[2-(4-benzyloxycarbonylpiperazin-1-yl)ethoxy]pyrimidin-2-yl]-3,8-diazabicyclo[3.2.1]octane-3-carboxylate

To a solution of benzyl 4-[2-(2-chloropyrimidin-4-yl)oxyethyl]piperazine-1-carboxylate (0.35 g, 0.93 mmol, 1.0 eq) in DMA (6.3 mL) was added tert-butyl 3,8-diazabicyclo[3.2.1]octane-3-carboxylate (0.21 g, 0.98 mmol, 1.05 eq) and potassium carbonate (0.26 g, 1.86 mmol, 2.0 eq). The reaction mixture was heated to 100° C. for 16 h. The reaction mixture was poured into a saturated NaHCO₃ solution and extracted with EtOAc. The combined organic layers were dried over MgSO₄ and concentrated in vacuo. The crude material was purified on silica column (heptane/EtOAc 0-100%) as eluent to afford the title compound (0.46 g, 0.83 mmol, 88% yield) as a colorless oil. MS (ESI): 553.5 ([M+H]⁺).

### c) Benzyl 4-[2-[2-(3,8-diazabicyclo[3.2.1]octan-8-yl)pyrimidin-4-yl]oxyethyl]piperazine-1-carboxylate

Tert-Butyl 8-[4-[2-(4-benzyloxycarbonylpiperazin-1-yl)ethoxy]pyrimidin-2-yl]-3,8-diazabicyclo[3.2.1]octane-3-carboxylate (150 mg, 271 μmol, 1.0 eq) was dissolved in DCM (1.5 mL) and HCl (0.68 mL, 2.71 mmol, 10.0 eq; 4 M solution in dioxane) was slowly added. The reaction mixture was stirred at ambient temperature for 4 h. The reaction mixture was concentrated in vacuo to afford the title compound (166 mg, 367 μmol, 125% yield) as an off-white solid, hydrochloride salt. MS (ESI): 453.5 ([M+H]⁺).

### d) Benzyl 4-[2-[2-[3-(3-amino-6-chloro-pyridazin-4-yl)-3,8-diazabicyclo[3.2.1]octan-8-yl]pyrimidin-4-yl]oxyethyl]piperazine-1-carboxylate

To a solution of benzyl 4-[2-[2-(3,8-diazabicyclo[3.2.1]octan-8-yl)pyrimidin-4-yl]oxyethyl]piperazine-1-carboxylate; hydrochloride (163 mg, 334 μmol, 1.0 eq) in DMSO (0.5 mL) was added 4-bromo-6-chloropyridazin-3-amine (77 mg, 367 μmol, 1.1 eq) and DIPEA (108 mg, 146 μL, 835 μmol, 3.0 eq). The reaction mixture was heated to 100° C. for 48 h. The reaction mixture was poured into a saturated NH₄Cl solution, extracted with EtOAc, washed with brine and the organic layer was dried over MgSO₄ and concentrated in vacuo. The crude material was purified by silica gel flash chromatography using DCM/MeOH (0-10%) as eluent to afford the title compound (60 mg, 103 μmol, 31% yield) as an orange foam. MS (ESI): 580.2542 ([M+H]⁺).

### e) Benzyl 4-[2-[2-[3-[3-amino-6-(2-hydroxyphenyl)pyridazin-4-yl]-3,8-diazabicyclo[3.2.1]octan-8-yl]pyrimidin-4-yl]oxyethyl]piperazine-1-carboxylate

To a solution of benzyl 4-[2-[2-[3-(3-amino-6-chloro-pyridazin-4-yl)-3,8-diazabicyclo[3.2.1]octan-8-yl]pyrimidin-4-yl]oxyethyl]piperazine-1-carboxylate (55 mg, 95 μmol, 1.0 eq) in a mixture of dioxane (2.5 mL) and water (0.25 mL) was added (2-hydroxyphenyl)boronic acid (32.7 mg, 237 μmol, 2.5 eq) and potassium carbonate (45.9 mg, 332 μmol, 3.5 eq). The solution was degassed by purging with argon and after addition of RuPhos Pd G3 (7.9 mg, 9.5 μmol, 0.1 eq) the reaction mixture was stirred at 90° C. for 2 h. The reaction mixture was poured into a saturated NH₄Cl solution, extracted with EtOAc, washed with brine and the organic layer was dried over Na₂SO₄ and concentrated in vacuo. The crude material was purified on silica column (DCM/MeOH 0-10%) to afford the title compound (26 mg, 40.8 μmol, 43% yield) as a yellow oil. MS (ESI): 638.6 ([M+H]⁺).

### f) 2-[6-Amino-5-[8-[4-(2-piperazin-1-ylethoxy)pyrimidin-2-yl]-3,8-diazabicyclo[3.2.1]octan-3-yl]pyridazin-3-yl]phenol

Benzyl 4-[2-[2-[3-[3-amino-6-(2-hydroxyphenyl)pyridazin-4-yl]-3,8-diazabicyclo[3.2.1]octan-8-yl]pyrimidin-4-yl]oxyethyl]piperazine-1-carboxylate (25 mg, 39 μmol, 1.0 eq) was dissolved in a mixture of methanol (0.5 mL) and THF (0.1 mL). The solution was degassed by purging with argon, Pd/C 10% (0.63 mg, 5.9 μmol, 0.15 eq) was added and the reaction mixture was stirred at ambient temperature for 8 h under an atmosphere of hydrogen (1 bar). The reaction mixture was filtered over dicalite and concentrated in vacuo to afford the title compound (18 mg, 35.7 μmol, 93% yield) as a yellow oil. MS (ESI): 504.4 ([M+H]⁺).

### g) rac-5-[4-[3-[4-[2-[2-[3-[3-Amino-6-(2-hydroxyphenyl)pyridazin-4-yl]-3,8-diazabicyclo[3.2.1]octan-8-yl]pyrimidin-4-yl]oxyethyl]piperazin-1-yl]propoxy]-1-piperidyl]-2-(2,6-dioxo-3-piperidyl)isoindoline-1,3-dione

2-[6-Amino-5-[8-[4-(2-piperazin-1-ylethoxy)pyrimidin-2-yl]-3,8-diazabicyclo[3.2.1]octan-3-yl]pyridazin-3-yl]phenol (15.5 mg, 30.7 μmol, 1.0 eq) was combined with Ligase 27 (14.7 mg, 30.7 μmol, 1.0 eq) and DIPEA (19.8 mg, 27 μL, 153 μmol, 5.0 eq) in DMF (250 μL). The yellow solution was stirred at 60° C. for 16 h and then purified by prep-HPLC to afford the title compound (15 mg, 33.3 μmol, 54% yield) as a light yellow solid. MS (ESI): 451.2272 ([M+2H]²⁺).

### Example 162

### 5-(2-(2-(1-(3-amino-6-(2-hydroxyphenyl)pyridazin-4-yl)-4-phenylpiperidine-4-carbonyl)-2,7-diazaspiro[3.5]nonan-7-yl)-2-oxoethoxy)-2-(2,6-dioxopiperidin-3-yl)isoindoline-1,3-dione

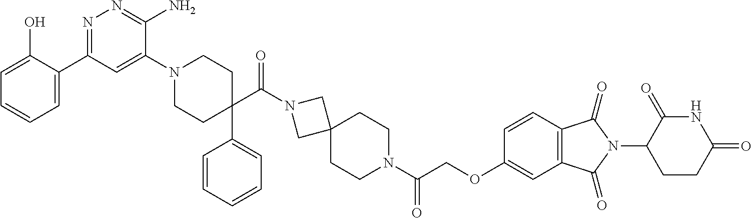 [SMILES: Nc1nnc(-c2ccccc2O)cc1N1CCC(C(=O)N2CC3(CCN(C(=O)COc4ccc5c(c4)C(=O)N(C4CCC(=O)NC4=O)C5=O)CC3)C2)(c2ccccc2)CC1]

### a) tert-butyl 2-[i-(3-amino-6-chloro-pyridazin-4-yl)-4-phenyl-piperidine-4-carbonyl]-2,7-diazaspiro[3.5]nonane-7-carboxylate

To a solution of potassium 4-(3-amino-6-chloro-pyridazin-4-yl)-1-phenyl-piperazine-2-carboxylate (195 mg, 526 μmol, 1.0 eq), HATU (240 mg, 380 μmol, 1.2 eq) and DIPEA (340 mg, 459 μL, 2.63 mmol, 5.0 eq) in DMF (2 mL) was added tert-butyl 2,7-diazaspiro[3.5]nonane-7-carboxylate (179 mg, 789 μmol, 1.5 eq). The reaction mixture was stirred at room temperature for 1 h. The reaction mixture was concentrated in vacuo. The crude material was purified on silica column (DCM/MeOH 0-10%) to afford the title compound (280 mg, 518 μmol, 98% yield) as light brown solid. MS (ESI): 541.3 ([M+H]⁺).

### b) tert-butyl 2-[1-[3-amino-6-(2-hydroxyphenyl)pyridazin-4-yl]-4-phenyl-piperidine-4-carbonyl]-2,7-diazaspiro[3.5]nonane-7-carboxylate

A suspension of tert-butyl 2-(1-(3-amino-6-chloropyridazin-4-yl)-4-phenylpiperidine-4-carbonyl)-2,7-diazaspiro[3.5]nonane-7-carboxylate (330 mg, 610 μmol, 1.0 eq), (2-hydroxyphenyl)boronic acid (210 mg, 1.52 mmol, 2.5 eq), potassium carbonate (253 mg, 1.83 mmol, 3.0 eq) and RuPhos Pd G3 (25.5 mg, 30.5 μmol, 0.05 eq) in a mixture of degassed dioxane (8 mL) and water (0.8 mL) was stirred at 110° C. for 2 h under argon.

The reaction mixture was poured in saturated NaHCO₃ and extracted with EtOAc. The organic layers were combined, washed with water and brine. The organic layers were dried over Na₂SO₄ and concentrated in vacuo. The crude material was purified on silica column (DCM/MeOH 0-10%) to afford the title compound (215 mg, 360 μmol, 59% yield) as a light brown solid. MS (ESI): 599.5 ([M+H]⁺).

### c) [1-[3-amino-6-(2-hydroxyphenyl)pyridazin-4-yl]-4-phenyl-4-piperidyl]-(2,7-diazaspiro[3.5]nonan-2-yl)methanone

A solution of tert-butyl 2-(1-(3-amino-6-(2-hydroxyphenyl)pyridazin-4-yl)-4-phenylpiperidine-4-carbonyl)-2,7-diazaspiro[3.5]nonane-7-carboxylate (200 mg, 334 μmol, 1.0 eq) in 1,1,1,3,3,3-hexafluoro-2-propanol (2.81 g, 1.75 ml, 16.7 mmol, 50 eq) in a sealed tube was stirred at 120° C. for 72 h. The reaction mixture was cooled to room temperature and concentrated in vacuo to afford the title compound (150 mg, 300 μmol, 90% yield) as a light brown solid. MS (ESI): 499.4 ([M+H]⁺).

### d) 5-(2-(2-(1-(3-amino-6-(2-hydroxyphenyl)pyridazin-4-yl)-4-phenylpiperidine-4-carbonyl)-2,7-diazaspiro[3.5]nonan-7-yl)-2-oxoethoxy)-2-(2,6-dioxopiperidin-3-yl)isoindoline-1,3-dione

Ligase 48 (12 mg, 36.1 μmol, 1.2 eq) was dissolved in dry DMF (400 μL). DIPEA (9.72 mg, 13.1 μL, 75.2 μmol, 2.5 eq) and HATU (14.9 mg, 39.1 μmol, 1.3 eq) were added and the mixture was stirred at room temperature for 10 min. (1-(3-amino-6-(2-hydroxyphenyl)pyridazin-4-yl)-4-phenylpiperidin-4-yl)(2,7-diazaspiro[3.5]nonan-2-yl)methanone (15 mg, 30.1 μmol, 1.0 eq) was added and the mixture was stirred at room temperature for 2 h. The reaction mixture was purified by prep-HPLC to afford the title compound (12.7 mg, 14 μmol, 46% yield) as a yellow solid, formate salt. MS (ESI): 813.6 ([M+H]⁺).

### Example 163

### 5-(4-(2-(1-(3-amino-6-(2-hydroxyphenyl)pyridazin-4-yl)-4-phenylpiperidine-4-carbonyl)-2,7-diazaspiro[3.5]nonane-7-carbonyl)piperidin-1-yl)-2-(2,6-dioxopiperidin-3-yl)isoindoline-1,3-dione

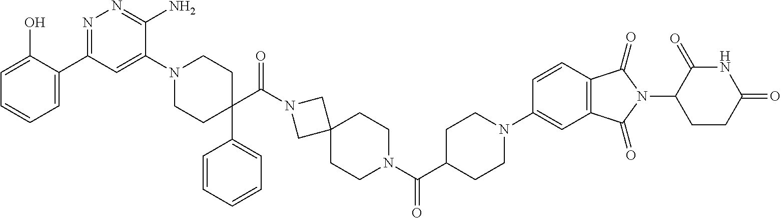 [SMILES: Nc1nnc(-c2ccccc2O)cc1N1CCC(C(=O)N2CC3(CCN(C(=O)C4CCN(c5ccc6c(c5)C(=O)N(C5CCC(=O)NC5=O)C6=O)CC4)CC3)C2)(c2ccccc2)CC1]

The title compound was prepared in analogy to example 162 step d using Ligase 15 as a yellow solid (11.8 mg, 11.6 μmol, 38% yield), formate salt. MS (ESI): 866.6 ([M+H]⁺).

### Example 164

### 5-(4-(2-(2-(1-(3-amino-6-(2-hydroxyphenyl)pyridazin-4-yl)-4-phenylpiperidine-4-carbonyl)-2,7-diazaspiro[3.5]nonan-7-yl)-2-oxoethyl)piperidin-1-yl)-2-(2,6-dioxopiperidin-3-yl)isoindoline-1,3-dione

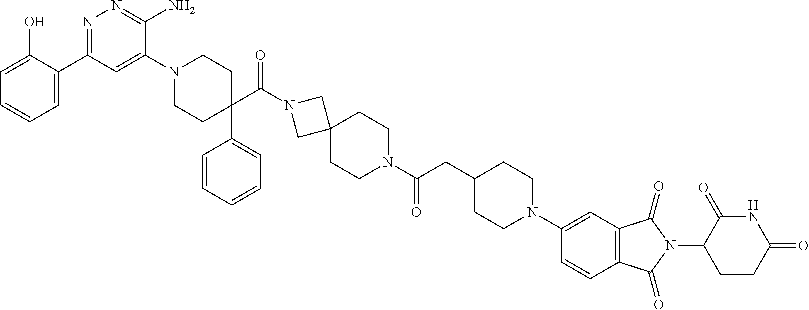 [SMILES: Nc1nnc(-c2ccccc2O)cc1N1CCC(C(=O)N2CC3(CCN(C(=O)CC4CCN(c5ccc6c(c5)C(=O)N(C5CCC(=O)NC5=O)C6=O)CC4)CC3)C2)(c2ccccc2)CC1]

The title compound was prepared in analogy to example 162 step d using Ligase 24 as a yellow solid (14.2 mg, 14.6 μmol, 48% yield), formate salt. MS (ESI): 880.7 ([M+H]⁺).

### Example 165

### 3-((4-(1-(2-(2-(1-(3-amino-6-(2-hydroxyphenyl)pyridazin-4-yl)-4-phenylpiperidine-4-carbonyl)-2,7-diazaspiro[3.5]nonan-7-yl)-2-oxoethyl)piperidin-4-yl)phenyl)amino)piperidine-2,6-dione

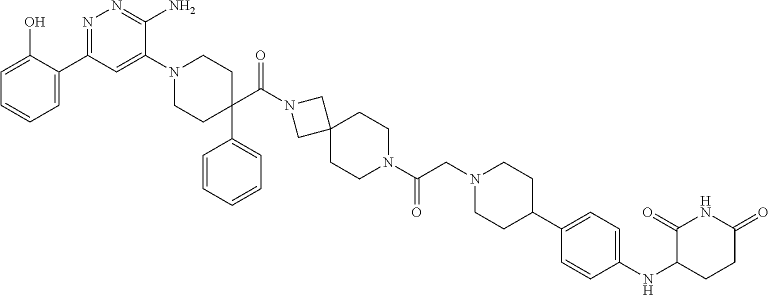 [SMILES: Nc1nnc(-c2ccccc2O)cc1N1CCC(C(=O)N2CC3(CCN(C(=O)CN4CCC(c5ccc(NC6CCC(=O)NC6=O)cc5)CC4)CC3)C2)(c2ccccc2)CC1]

The title compound was prepared in analogy to example 162 step d using Ligase 51 as a yellow solid (10.3 mg, 10.6 μmol, 35% yield), formate salt. MS (ESI): 826.5 ([M+H]⁺).

### Example 166

### 5-(2-(9-(1-(3-amino-6-(2-hydroxyphenyl)pyridazin-4-yl)-4-phenylpiperidine-4-carbonyl)-1-oxa-4,9-diazaspiro[5.5]undecan-4-yl)-2-oxoethoxy)-2-(2,6-dioxopiperidin-3-yl)isoindoline-1,3-dione

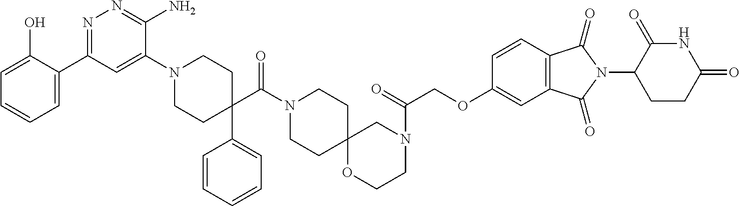 [SMILES: Nc1nnc(-c2ccccc2O)cc1N1CCC(C(=O)N2CCC3(CC2)CN(C(=O)COc2ccc4c(c2)C(=O)N(C2CCC(=O)NC2=O)C4=O)CCO3)(c2ccccc2)CC1]

### a) tert-butyl 9-[1-(3-amino-6-chloro-pyridazin-4-yl)-4-phenyl-piperidine-4-carbonyl]-1-oxa-4,9-diazaspiro[5.5]undecane-4-carboxylate

To a solution of potassium 4-(3-amino-6-chloro-pyridazin-4-yl)-1-phenyl-piperazine-2-carboxylate (190 mg, 511 μmol, 1.0 eq), HATU (233 mg, 613 μmol, 1.2 eq) and DIPEA (330 mg, 446 μL, 2.55 mmol, 5.0 eq) in DMF (3 mL) was added tert-butyl 1-oxa-4,9-diazaspiro[5.5]undecane-4-carboxylate (196 mg, 766 μmol, 1.5 eq). The reaction mixture was stirred at room temperature for 1 h. The reaction mixture was concentrated in vacuo. The crude material was purified on silica column (DCM/MeOH 0-10%) to afford the title compound (250 mg, 439 μmol, 86% yield) as a light brown solid. MS (ESI): 571.4 ([M+H]⁺).

### b) tert-butyl 9-[1-[3-amino-6-(2-hydroxyphenyl)pyridazin-4-yl]-4-phenyl-piperidine-4-carbonyl]-1-oxa-4,9-diazaspiro[5.5]undecane-4-carboxylate

A suspension of tert-butyl 9-(1-(3-amino-6-chloropyridazin-4-yl)-4-phenylpiperidine-4-carbonyl)-1-oxa-4,9-diazaspiro[5.5]undecane-4-carboxylate (330 mg, 578 μmol, 1.0 eq), (2-hydroxyphenyl)boronic acid (199 mg, 1.44 mmol, 2.5 eq), potassium carbonate (240 mg, 1.73 mmol, 3.0 eq) and RuPhos Pd G3 (24.2 mg, 28.9 μmol, 0.05 eq) in a mixture of degassed dioxane (8 mL) and water (0.8 mL) was stirred at 110° C. for 2 h under argon. The reaction mixture was poured in saturated NaHCO₃ and extracted with EtOAc. The organic layers were combined, washed with water and brine. The organic layers were dried over Na₂SO₄ and concentrated in vacuo. The crude material was purified on silica column (heptane/EtOAc 0-100%) to afford the title compound (275 mg, 437 μmol, 76% yield) as an off-white solid. MS (ESI): 629.5 ([M+H]⁺).

### c) [1-[3-amino-6-(2-hydroxyphenyl)pyridazin-4-yl]-4-phenyl-4-piperidyl]-(1-oxa-4,9-diazaspiro[5.5]undecan-9-yl)methanone

To a cooled (0° C.) solution of tert-butyl 9-(1-(3-amino-6-(2-hydroxyphenyl)pyridazin-4-yl)-4-phenylpiperidine-4-carbonyl)-1-oxa-4,9-diazaspiro[5.5]undecane-4-carboxylate (260 mg, 414 μmol, 1.0 eq) in DCM (4 mL) was added 4M HCl in dioxane (310 μL, 1.24 mmol, 3.0 eq). The reaction mixture was allowed to reach room temperature and stirred for 16 h. The reaction mixture was concentrated in vacuo to afford the title compound (230 mg, 407 μmol, 98% yield) as a white solid, hydrochloride salt. MS (ESI): 529.4 ([M+H]⁺).

### d) 5-(2-(9-(1-(3-amino-6-(2-hydroxyphenyl)pyridazin-4-yl)-4-phenylpiperidine-4-carbonyl)-1-oxa-4,9-diazaspiro[5.5]undecan-4-yl)-2-oxoethoxy)-2-(2,6-dioxopiperidin-3-yl)isoindoline-1,3-dione

Ligase 48 (10.6 mg, 31.9 μmol, 1.2 eq) was dissolved in dry DMF (400 μL). DIPEA (8.58 mg, 11.6 μL, 66.4 μmol, 2.5 eq) and HATU (13.1 mg, 34.5 μmol, 1.3 eq) were added and the mixture was stirred at room temperature for 10 min. (1-(3-amino-6-(2-hydroxyphenyl)pyridazin-4-yl)-4-phenylpiperidin-4-yl)(1-oxa-4,9-diazaspiro[5.5]undecan-9-yl)methanone, hydrochloride salt (15 mg, 26.5 μmol, 1.0 eq) was added and the mixture was stirred at room temperature for 2 h. The reaction mixture was purified directly by prep-HPLC to afford the title compound (11.3 mg, 11.4 μmol, 43% yield) as a yellow solid, formate salt. MS (ESI): 843.5 ([M+H]⁺).

### Example 167

### 5-(4-(9-(1-(3-amino-6-(2-hydroxyphenyl)pyridazin-4-yl)-4-phenylpiperidine-4-carbonyl)-1-oxa-4,9-diazaspiro[5.5]undecane-4-carbonyl)piperidin-1-yl)-2-(2,6-dioxopiperidin-3-yl)isoindoline-1,3-dione

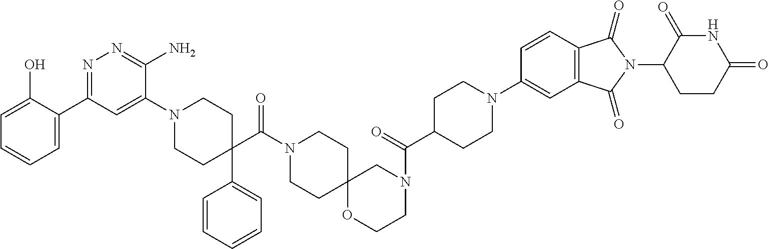 [SMILES: Nc1nnc(-c2ccccc2O)cc1N1CCC(C(=O)N2CCC3(CC2)CN(C(=O)C2CCN(c4ccc5c(c4)C(=O)N(C4CCC(=O)NC4=O)C5=O)CC2)CCO3)(c2ccccc2)CC1]

The title compound was prepared in analogy to example 166 step d using Ligase 15 as a yellow solid (19.8 mg, 18.9 μmol, 71% yield), formate salt. MS (ESI): 896.6 ([M+H]⁺).

### Example 168

### 5-(4-(2-(9-(1-(3-amino-6-(2-hydroxyphenyl)pyridazin-4-yl)-4-phenylpiperidine-4-carbonyl)-1-oxa-4,9-diazaspiro[5.5]undecan-4-yl)-2-oxoethyl)piperidin-1-yl)-2-(2,6-dioxopiperidin-3-yl)isoindoline-1,3-dione

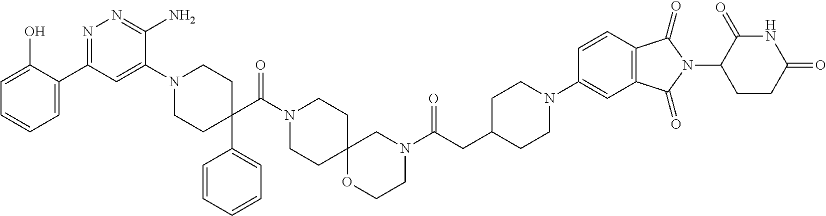 [SMILES: Nc1nnc(-c2ccccc2O)cc1N1CCC(C(=O)N2CCC3(CC2)CN(C(=O)CC2CCN(c4ccc5c(c4)C(=O)N(C4CCC(=O)NC4=O)C5=O)CC2)CCO3)(c2ccccc2)CC1]

The title compound was prepared in analogy to example 166 step d using Ligase 24 as a yellow solid (17.1 mg, 17 μmol, 64% yield), formate salt. MS (ESI): 910.3 ([M+H]⁺).

### Example 169

### 3-((4-(1-(2-(9-(1-(3-amino-6-(2-hydroxyphenyl)pyridazin-4-yl)-4-phenylpiperidine-4-carbonyl)-1-oxa-4,9-diazaspiro[5.5]undecan-4-yl)-2-oxoethyl)piperidin-4-yl)phenyl)amino)piperidine-2,6-dione

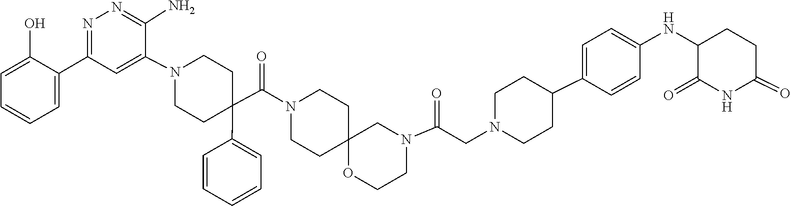 [SMILES: Nc1nnc(-c2ccccc2O)cc1N1CCC(C(=O)N2CCC3(CC2)CN(C(=O)CN2CCC(c4ccc(NC5CCC(=O)NC5=O)cc4)CC2)CCO3)(c2ccccc2)CC1]

The title compound was prepared in analogy to example 166 step d using Ligase 51 as a yellow solid (5.8 mg, 6.11 μmol, 23% yield), formate salt. MS (ESI): 856.7 ([M+H]⁺).

### Example 170

### rac-1-[[3-[3-[3-amino-6-(2-hydroxyphenyl)pyridazin-4-yl]-3,8-diazabicyclo[3.2.1]octan-8-yl]phenyl]methyl]-N-[1-[2-(2,6-dioxo-3-piperidyl)-1,3-dioxo-isoindolin-5-yl]azetidin-3-yl]-N-methyl-piperidine-4-carboxamide

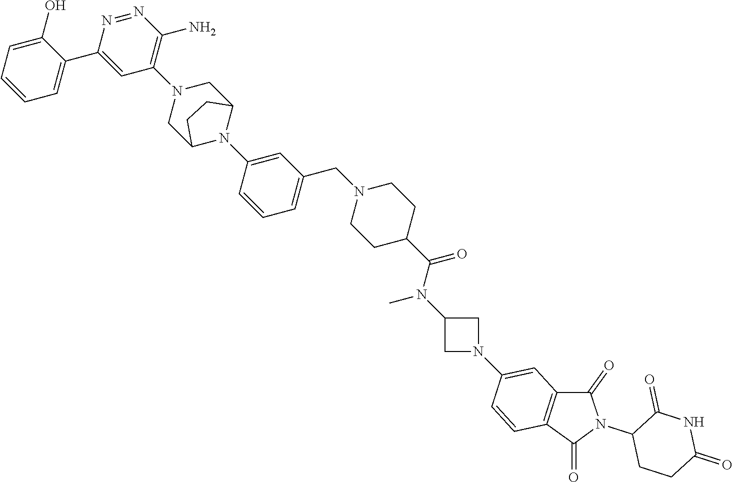 [SMILES: CN(C(=O)C1CCN(Cc2cccc(N3C4CCC3CN(c3cc(-c5ccccc5O)nnc3N)C4)c2)CC1)C1CN(c2ccc3c(c2)C(=O)N(C2CCC(=O)NC2=O)C3=O)C1]

To a stirred solution of 1-[[3-[3-[3-amino-6-(2-hydroxyphenyl)pyridazin-4-yl]-3,8-diazabicyclo[3.2.1]octan-8-yl]phenyl]methyl]piperidine-4-carboxylic acid (60 mg, 117 μmol, 1.0 eq), Ligase 60 (39.9 mg, 117 μmol, 1.0 eq) and DIPEA (75.3 mg, 102 μL, 583 μmol, 5.0 eq) in DMSO (0.6 mL) at room temperature was added HATU (88.7 mg, 233 μmol, 2.0 eq). The reaction mixture was stirred at room temperature for 1 h. The crude material was purified by prep-HPLC to afford the title compound (20 mg, 23.8 μmol, 20% yield) as a yellow solid. MS (ESI): 837.6 ([M−H]⁻).

### Example 171

### rac-2-(2,6-dioxo-3-piperidyl)-5-[rel-(3aR,6aS)-5-[1-[[3-[3-[3-amino-6-(2-hydroxyphenyl)pyridazin-4-yl]-3,8-diazabicyclo[3.2.1]octan-8-yl]phenyl]methyl]piperidine-4-carbonyl]-1,3,3a,4,6,6a-hexahydropyrrolo[3,4-c]pyrrol-2-yl]isoindoline-1,3-dione

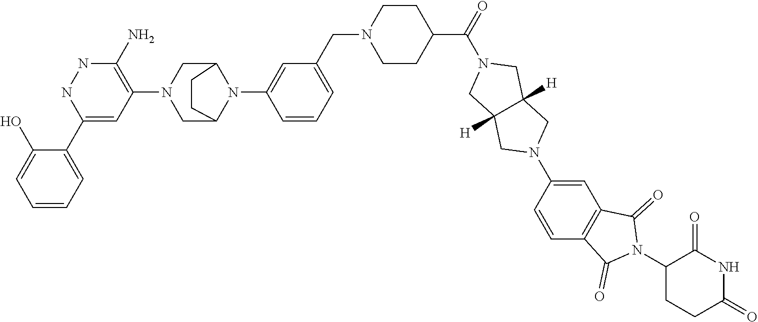 [SMILES: NC1=C(N2CC3CCC(C2)N3c2cccc(CN3CCC(C(=O)N4C[C@@H]5CN(c6ccc7c(c6)C(=O)N(C6CCC(=O)NC6=O)C7=O)C[C@@H]5C4)CC3)c2)C=C(c2ccccc2O)NN1]

To a stirred solution of 1-[[3-[3-[3-amino-6-(2-hydroxyphenyl)pyridazin-4-yl]-3,8-diazabicyclo[3.2.1]octan-8-yl]phenyl]methyl]piperidine-4-carboxylic acid (25 mg, 48.6 μmol, 1.0 eq), Ligase 67 (28 mg, 34.6 μmol, 0.712 eq) and DIPEA (37.7 mg, 50.9 μL, 291 μmol, 6.0 eq) in DMSO (0.4 mL) at room temperature was added HATU (36.9 mg, 97.2 μmol, 2.0 eq). The reaction mixture was stirred at room temperature for 1 h. The crude material was purified by prep-HPLC to afford the title compound (7 mg, 5.76 μmol, 12% yield) as a yellow solid, bis-(2,2,2-trifluoroacetic acid) salt. MS: 865.7 ([M+H]⁺).

### Example 172

### 3-(4-(1-(4-(2-(1-(3-amino-6-(2-hydroxyphenyl)pyridazin-4-yl)-4-phenylpiperidine-4-carbonyl)-2,7-diazaspiro[3.5]nonan-7-yl)-4-oxobutyl)piperidin-4-yl)phenoxy)piperidine-2,6-dione

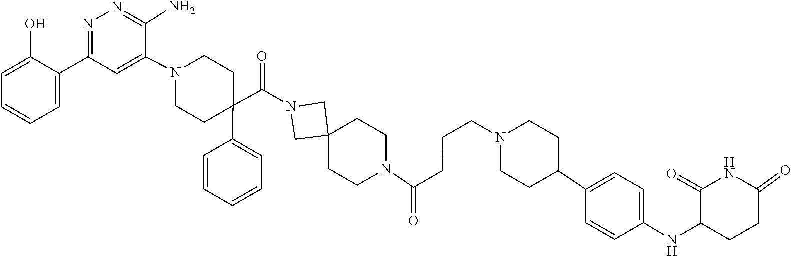 [SMILES: Nc1nnc(-c2ccccc2O)cc1N1CCC(C(=O)N2CC3(CCN(C(=O)CCCN4CCC(c5ccc(NC6CCC(=O)NC6=O)cc5)CC4)CC3)C2)(c2ccccc2)CC1]

The title compound was prepared in analogy to example 162 step d using Ligase 39 as a yellow solid (3.6 mg, 3.8 μmol, 12% yield), formate salt. MS (ESI): 853.8 ([M+H]⁺).

### Example 173

### 3-((4-(4-(2-(9-(1-(3-amino-6-(2-hydroxyphenyl)pyridazin-4-yl)-4-phenylpiperidine-4-carbonyl)-1-oxa-4,9-diazaspiro[5.5]undecan-4-yl)-2-oxoethyl)piperazin-1-yl)phenyl)amino)piperidine-2,6-dione

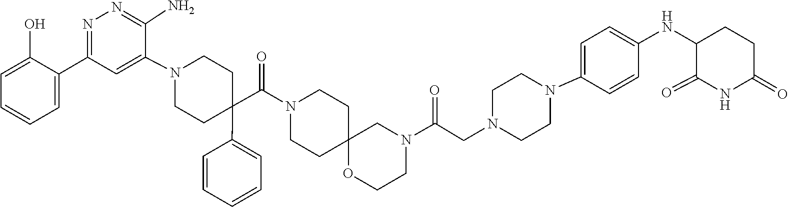 [SMILES: Nc1nnc(-c2ccccc2O)cc1N1CCC(C(=O)N2CCC3(CC2)CN(C(=O)CN2CCN(c4ccc(NC5CCC(=O)NC5=O)cc4)CC2)CCO3)(c2ccccc2)CC1]

The title compound was prepared in analogy to example 166 step d using Ligase 54 as a yellow solid (3.8 mg, 3.96 μmol, 14% yield), formate salt. MS (ESI): 855.8 ([M+H]⁺).

### Example 174

### 5-((4-(1-(1-(3-amino-6-(2-hydroxyphenyl)pyridazin-4-yl)-4-phenylpiperidine-4-carbonyl)piperidine-4-carbonyl)piperazin-1-yl)methyl)-2-(2,6-dioxopiperidin-3-yl)isoindoline-1,3-dione

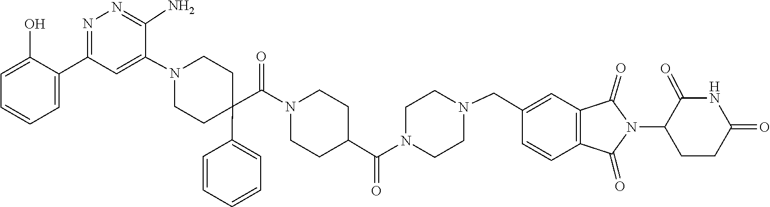 [SMILES: Nc1nnc(-c2ccccc2O)cc1N1CCC(C(=O)N2CCC(C(=O)N3CCN(Cc4ccc5c(c4)C(=O)N(C4CCC(=O)NC4=O)C5=O)CC3)CC2)(c2ccccc2)CC1]

### a) benzyl 4-(4-ethoxycarbonylpiperidine-1-carbonyl)-4-phenyl-piperidine-1-carboxylate

To a solution of 1-((benzyloxy)carbonyl)-4-phenylpiperidine-4-carboxylic acid (500 mg, 1.47 mmol, 1.0 eq), HATU (672 mg, 1.77 mmol, 1.2 eq) and DIPEA (1.90 g, 2.57 mL, 14.7 mmol, 10.0 eq) in DMF (8 mL) was added ethyl piperidine-4-carbonate (347 mg, 341 μL, 2.21 mmol, 1.5 eq). The reaction mixture was stirred at room temperature for 16 h. The reaction mixture was poured into saturated NH₄Cl and extracted with EtOAc. The combined organic layers were washed with water, brine, dried over Na₂SO₄ and concentrated under reduced pressure. The crude material was purified on silica column (heptane/EtOAc 0-80%) to afford the title compound (621 mg, 1.30 mmol, 88% yield) as a white powder. MS (ESI): 479.4 ([M+H]⁺).

### b) ethyl 1-(4-phenylpiperidine-4-carbonyl)piperidine-4-carboxylate

To a solution of benzyl 4-(4-(ethoxycarbonyl)piperidine-1-carbonyl)-4-phenylpiperidine-1-carboxylate (620 mg, 1.3 mmol, 1.0 eq) in methanol (8 mL) was added 10% palladium on charcoal (138 mg, 130 μmol, 0.1 eq). The reaction mixture was vigorously stirred at room temperature for 3 h under H₂ (baloon). The catalyst was collected by filtration, washing with methanol. The filtrate was concentrated to afford the title compound (421 mg, 1.22 mmol, 94% yield) as a light brown solid. MS (ESI): 345.3 ([M+H]⁺).

### c) ethyl 1-[1-(3-amino-6-chloro-pyridazin-4-yl)-4-phenyl-piperidine-4-carbonyl]piperidine-4-carboxylate

To a stirred solution of 4-bromo-6-chloropyridazin-3-amine (266 mg, 1.28 mmol, 1.0 eq) and ethyl 1-(4-phenylpiperidine-4-carbonyl)piperidine-4-carboxylate (0.44 g, 1.28 mmol, 1.0 eq) in DMA (7 mL) was added potassium carbonate (1.06 g, 7.66 mmol, 6.0 eq). The reaction mixture was heated to 110° C. and stirred for 20 h. The reaction mixture was poured in water and extracted with EtOAc. The organic layers were combined, washed with saturated NaHCO₃, water and brine. The organic layers were dried over Na₂SO₄ and concentrated in vacuo. The crude material was purified on silica column (heptane/EtOAc 0-100%) to afford the title compound (550 mg, 1.17 mmol, 91% yield) as a dark brown solid. MS (ESI): 472.4 ([M+H]⁺).

### d) ethyl 1-[1-[3-amino-6-(2-hydroxyphenyl)pyridazin-4-yl]-4-phenyl-piperidine-4-carbonyl]piperidine-4-carboxylate

A suspension of ethyl 1-(1-(3-amino-6-chloropyridazin-4-yl)-4-phenylpiperidine-4-carbonyl)piperidine-4-carboxylate (500 mg, 1.06 mmol, 1.0 eq), (2-hydroxyphenyl)boronic acid (365 mg, 2.65 mmol, 2.5 eq), potassium carbonate (512 mg, 3.71 mmol, 3.5 eq) and RuPhos Pd G3 (88.6 mg, 106 μmol, 0.05 eq) in a mixture of degassed dioxane (5 mL) and water (0.5 mL) was stirred at 110° C. for 3 h under argon. The reaction mixture was poured in saturated NaHCO₃ and extracted with EtOAc. The organic layers were combined, washed with water and brine. The organic layers were dried over Na₂SO₄ and concentrated in vacuo. The crude material was purified on silica column (DCM/MeOH 0-10%) to afford the title compound (531 mg, 1.0 mmol, 94% yield) as a dark brown solid. MS (ESI): 530.4 ([M+H]⁺).

### e) 1-[1-[3-amino-6-(2-hydroxyphenyl)pyridazin-4-yl]-4-phenyl-piperidine-4-carbonyl]piperidine-4-carboxylic acid

A solution of ethyl 1-(1-(3-amino-6-(2-hydroxyphenyl)pyridazin-4-yl)-4-phenylpiperidine-4-carbonyl)piperidine-4-carboxylate (520 mg, 982 μmol, 1.0 eq) in a mixture of THF (2.5 mL) and water (2.5 mL) was added lithium hydroxide (150 mg, 3.54 mmol, 3.6 eq) was stirred at room temperature for 24 h. The reaction mixture was pourred in saturated NH₄Cl and extracted with EtOAc. The organic layer was washed with water, brine, dried over Na₂SO₄ and concentrated to afford the title compound (389 mg, 776 μmol, 79% yield) as a brown solid. MS (ESI): 502.4 ([M+H]⁺).

### f) 5-((4-(1-(1-(3-amino-6-(2-hydroxyphenyl)pyridazin-4-yl)-4-phenylpiperidine-4-carbonyl)piperidine-4-carbonyl)piperazin-1-yl)methyl)-2-(2,6-dioxopiperidin-3-yl)isoindoline-1,3-dione

1-(1-(3-amino-6-(2-hydroxyphenyl)pyridazin-4-yl)-4-phenylpiperidine-4-carbonyl)piperidine-4-carboxylic acid (20 mg, 39.9 μmol, 1.0 eq) was dissolved in dry DMF (400 μL). DIPEA (12.9 mg, 17.4 μL, 99.7 μmol, 2.5 eq) and HATU (19.7 mg, 51.8 μmol, 1.3 eq) were added and the mixture was stirred at room temperature for 10 min. Ligase 68 (18.8 mg, 47.8 μmol, 1.2 eq) was added and the mixture was stirred at room temperature for 2 h. The reaction mixture was purified directly by prep-HPLC to afford the title compound (3 mg, 3.32 μmol, 8% yield) as a yellow solid, formate salt. MS (ESI): 838.7 ([M+H]⁺).

### Example 175

### 3-(4-(1-(1-(1-(3-amino-6-(2-hydroxyphenyl)pyridazin-4-yl)-4-phenylpiperidine-4-carbonyl)piperidine-4-carbonyl)piperidin-4-yl)phenyl)piperidine-2,6-dione

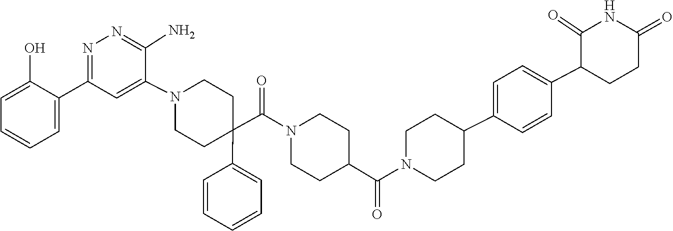 [SMILES: Nc1nnc(-c2ccccc2O)cc1N1CCC(C(=O)N2CCC(C(=O)N3CCC(c4ccc(C5CCC(=O)NC5=O)cc4)CC3)CC2)(c2ccccc2)CC1]

The title compound was prepared in analogy to example 174 step f using ligase 69 as a yellow solid (2.3 mg, 2.72 μmol, 6% yield), formate salt. MS (ESI): 756.6 ([M+H]⁺).

### Example 176

### 3-(4-(4-(1-(1-(3-amino-6-(2-hydroxyphenyl)pyridazin-4-yl)-4-phenylpiperidine-4-carbonyl)piperidine-4-carbonyl)piperazin-1-yl)phenyl)piperidine-2,6-dione

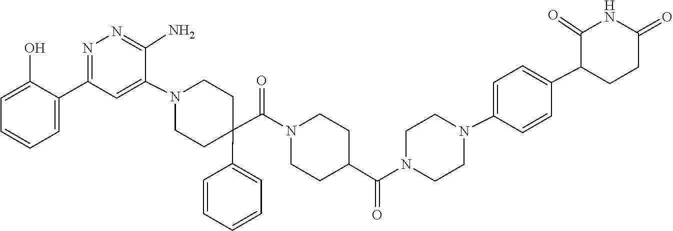 [SMILES: Nc1nnc(-c2ccccc2O)cc1N1CCC(C(=O)N2CCC(C(=O)N3CCN(c4ccc(C5CCC(=O)NC5=O)cc4)CC3)CC2)(c2ccccc2)CC1]

The title compound was prepared in analogy to example 174 step f using Ligase 70 as a yellow solid (1.5 mg, 1.77 μmol, 4% yield), formate salt. MS (ESI): 757.6 ([M+H]⁺).

### Example 177

### 3-((6-(1-(1-(1-(3-amino-6-(2-hydroxyphenyl)pyridazin-4-yl)-4-phenylpiperidine-4-carbonyl)piperidine-4-carbonyl)piperidin-4-yl)pyridin-3-yl)amino)piperidine-2,6-dione

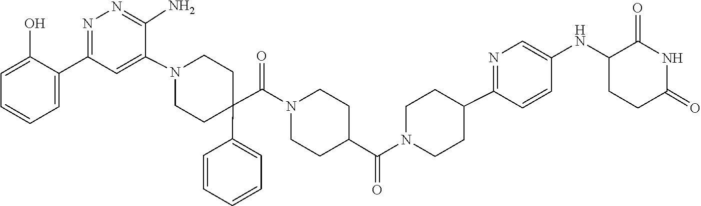 [SMILES: Nc1nnc(-c2ccccc2O)cc1N1CCC(C(=O)N2CCC(C(=O)N3CCC(c4ccc(NC5CCC(=O)NC5=O)cn4)CC3)CC2)(c2ccccc2)CC1]

The title compound was prepared in analogy to example 174 step f using Ligase 71 as a yellow solid (2 mg, 2.27 μmol, 5% yield), formate salt. MS (ESI): 772.5 ([M+H]⁺).

### Example 178

### 5-(4-(1-(1-(3-amino-6-(2-hydroxyphenyl)pyridazin-4-yl)-4-phenylpiperidine-4-carbonyl)piperidine-4-carbonyl)piperazin-1-yl)-2-(2,6-dioxopiperidin-3-yl)isoindoline-1,3-dione

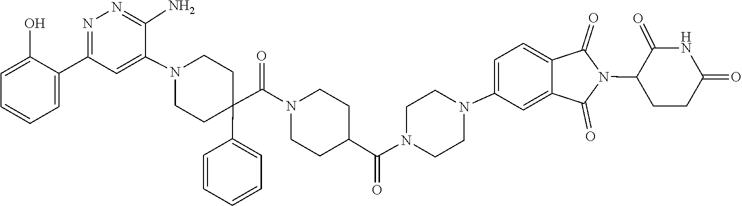 [SMILES: Nc1nnc(-c2ccccc2O)cc1N1CCC(C(=O)N2CCC(C(=O)N3CCN(c4ccc5c(c4)C(=O)N(C4CCC(=O)NC4=O)C5=O)CC3)CC2)(c2ccccc2)CC1]

The title compound was prepared in analogy to example 174 step f using Ligase 37 as a yellow solid (4 mg, 4.13 μmol, 10% yield), formate salt. MS (ESI): 826.6 ([M+H]⁺).

### Example 179

### rac-5-[3-[4-[[3-[3-[3-amino-6-(2-hydroxyphenyl)pyridazin-4-yl]-3,8-diazabicyclo[3.2.1]octan-8-yl]phenyl]methyl]piperazine-1-carbonyl]azetidin-1-yl]-2-(2,6-dioxo-3-piperidyl)isoindoline-1,3-dione

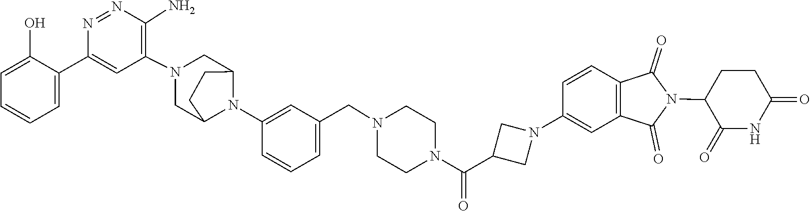 [SMILES: Nc1nnc(-c2ccccc2O)cc1N1CC2CCC(C1)N2c1cccc(CN2CCN(C(=O)C3CN(c4ccc5c(c4)C(=O)N(C4CCC(=O)NC4=O)C5=O)C3)CC2)c1]

To a crude solution of Ligase 44 (112 μmol) in DMSO were added 2-[6-amino-5-[8-[3-(piperazin-1-ylmethyl)phenyl]-3,8-diazabicyclo[3.2.1]octan-3-yl]pyridazin-3-yl]phenol dihydrochloride (81.5 mg, 112 μmol, 1.0 eq), DIPEA (72.5 mg, 98 μL, 561 μmol, 5.0 eq) and HATU (85.3 mg, 224 μmol, 2.0 eq). The reaction mixture was stirred at room temperature for 30 min. The crude material was purified by prep-HPLC. The product was lyophilized and the crude material was purified on silica column (DCM/MeOH 0-5%) to afford the title compound (11 mg, 12.9 μmol, 12% yield) as a yellow solid. MS (ESI): 811.7 ([M+H]⁺).

### Example 180

### 5-[4-[2-[4-[[3-[3-[3-amino-6-(2-hydroxyphenyl)pyridazin-4-yl]-3,8-diazabicyclo[3.2.1]octan-8-yl]phenyl]methyl]piperazin-1-yl]-2-oxo-ethyl]-1-piperidyl]-2-(2,6-dioxo-3-piperidyl)isoindoline-1,3-dione

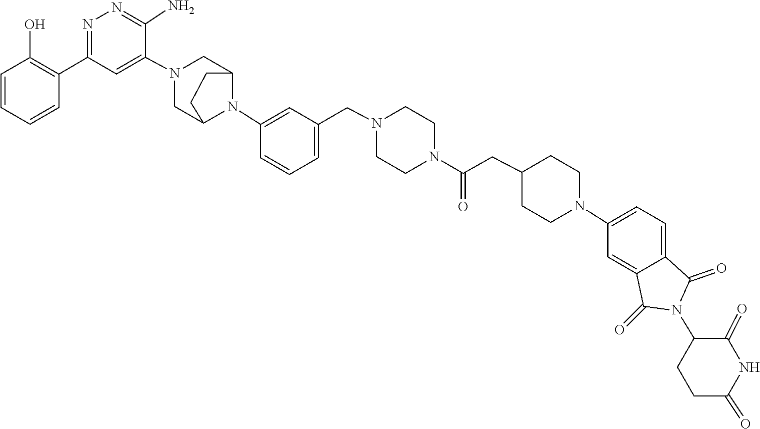 [SMILES: Nc1nnc(-c2ccccc2O)cc1N1CC2CCC(C1)N2c1cccc(CN2CCN(C(=O)CC3CCN(c4ccc5c(c4)C(=O)N(C4CCC(=O)NC4=O)C5=O)CC3)CC2)c1]

2-(1-(2-(2,6-dioxopiperidin-3-yl)-1,3-dioxoisoindolin-5-yl)piperidin-4-yl)acetic acid (29.3 mg, 73.5 μmol, 1.0 eq) was combined with DMF (0.8 mL). HATU (55.9 mg, 147 μmol, 2.0) and DIPEA (38 mg, 51.3 μL, 294 μmol, 4.0 eq) were added and the reaction mixture was stirred at room temperature for 30 min. 2-(6-amino-5-(8-(3-(piperazin-1-ylmethyl)phenyl)-3,8-diazabicyclo[3.2.1]octan-3-yl)pyridazin-3-yl)phenol dihydrochloride (40 mg, 73.5 μmol, 1.0 eq) was added and the reaction mixture was stirred at room temperature overnight. The crude material was purified by prep-HPLC to afford the title compound (28.4 mg, 33.3 μmol, 45% yield) as a light yellow lyoph solid. MS (ESI): 853.41 ([M+H]⁺).

### Example 181

### 3-(4-(1-(4-(9-(1-(3-amino-6-(2-hydroxyphenyl)pyridazin-4-yl)-4-phenylpiperidine-4-carbonyl)-1-oxa-4,9-diazaspiro[5.5]undecan-4-yl)-4-oxobutyl)piperidin-4-yl)phenoxy)piperidine-2,6-dione

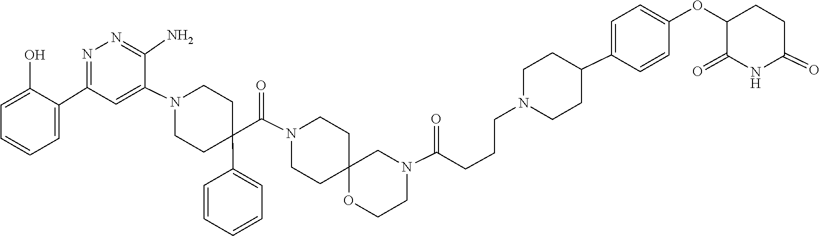 [SMILES: Nc1nnc(-c2ccccc2O)cc1N1CCC(C(=O)N2CCC3(CC2)CN(C(=O)CCCN2CCC(c4ccc(OC5CCC(=O)NC5=O)cc4)CC2)CCO3)(c2ccccc2)CC1]

The title compound was prepared in analogy to example 166 step d using Ligase 39 as a yellow solid (15.2 mg, 14.6 μmol, 55% yield), formate salt. MS (ESI): 885.7 ([M+H]⁺).

### Example 182

### N-[[1-[3-amino-6-(3,5-difluoro-2-hydroxy-phenyl)pyridazin-4-yl]-4-phenyl-4-piperidyl]methyl]-4-[4-[4-[(2,6-dioxo-3-piperidyl)oxy]phenyl]-1-piperidyl]butanamide

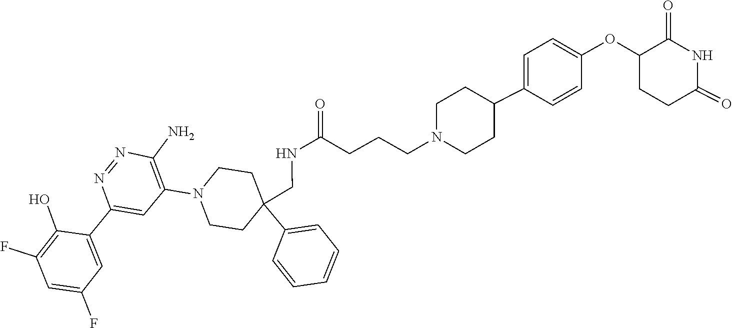 [SMILES: Nc1nnc(-c2cc(F)cc(F)c2O)cc1N1CCC(CNC(=O)CCCN2CCC(c3ccc(OC4CCC(=O)NC4=O)cc3)CC2)(c2ccccc2)CC1]

### a) tert-butyl ((1-(3-amino-6-(3,5-difluoro-2-hydroxyphenyl)pyridazin-4-yl)-4-phenylpiperidin-4-yl)methyl)carbamate

In a 10 mL three-necked flask flushed with Argon, tert-butyl ((1-(3-amino-6-chloropyridazin-4-yl)-4-phenylpiperidin-4-yl)methyl)carbamate (140 mg, 335 μmol, 1.0 eq), (3,5-difluoro-2-hydroxyphenyl)boronic acid (58.3 mg, 335 μmol, 1.0 eq) and K₂CO₃ (139 mg, 1 mmol, 3.0 eq), followed by methanesulfonato(2-dicyclohexylphosphino-2′,6′-di-1-propoxy-1,1′-biphenyl)(2′-amino-1,1′-biphenyl-2-yl)palladium(II) (RuPhos Pd G3) (8.41 mg, 10 μmol, 0.06 eq) were combined with degassed dioxane (3 mL)/water (0.3 mL). The reaction mixture was heated to 110° C. and stirred for 40 h. The crude material was purified on silica column (heptane/EtOAc 0-100%) to afford the title compound (95 mg, 186 μmol, 55% yield) as a light brown amorphous solid. MS (ESI): 512.2457 ([M+H]⁺).

### b) 2-(6-amino-5-(4-(aminomethyl)-4-phenylpiperidin-1-yl)pyridazin-3-yl)-4,6-difluorophenol

Tert-butyl ((1-(3-amino-6-(3,5-difluoro-2-hydroxyphenyl)pyridazin-4-yl)-4-phenylpiperidin-4-yl)methyl)carbamate (90 mg, 176 μmol, 1.0 eq) and 4N hydrogen chloride in dioxane (300 μL, 1.2 mmol, 6.82 eq) were combined with dioxane (1 mL). The reaction was stirred for 20 h. The precipitated solid was filtered off and washed with ether and dried under high vacuum, to afford the title compound (85 mg, 186 μmol, 106% yield) as a light yellow hydrochloride salt. MS (ESI): 412.1936 ([M+H]⁺).

### c) N-((1-(3-amino-6-(3,5-difluoro-2-hydroxyphenyl)pyridazin-4-yl)-4-phenylpiperidin-4-yl)methyl)-4-(4-(4-((2,6-dioxopiperidin-3-yl)oxy)phenyl)piperidin-1-yl)butanamide 2,2,2-trifluoroacetate

2-(6-amino-5-(4-(aminomethyl)-4-phenylpiperidin-1-yl)pyridazin-3-yl)-4,6-difluorophenol hydrochloride (12 mg, 26.8 μmol, 1.0 eq) and Ligase 39 (11 mg, 26.8 μmol, 1.0 eq) was stirred with DIPEA (10.4 mg, 14 μL, 80.4 μmol, 3.0 eq) in DMF (120 μL). HATU (12.2 mg, 32.1 μmol, 1.2 eq) was added and the mixture was stirred at room temperature overnight. The crude material was purified by prep-HPLC to afford the title compound (7 mg, 7.94 μmol, 29% yield) as a white solid, 2,2,2-trifluoroacetate salt. MS (ESI): 768.3677 ([M+H]⁺).

### Example 183

### N-[[1-[3-amino-6-(5-fluoro-2-hydroxy-phenyl)pyridazin-4-yl]-4-phenyl-4-piperidyl]methyl]-4-[4-[4-[(2,6-dioxo-3-piperidyl)oxy]phenyl]-1-piperidyl]butanamide

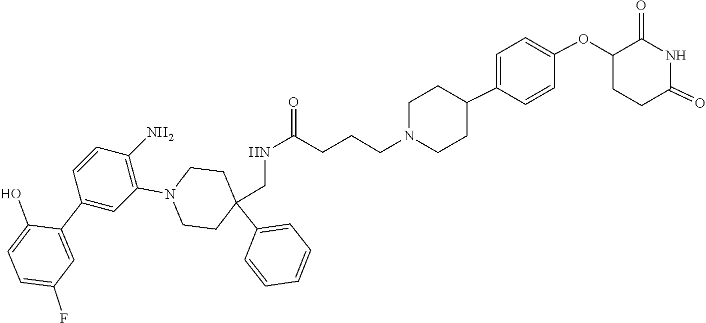 [SMILES: Nc1ccc(-c2cc(F)ccc2O)cc1N1CCC(CNC(=O)CCCN2CCC(c3ccc(OC4CCC(=O)NC4=O)cc3)CC2)(c2ccccc2)CC1]

### a) tert-butyl ((1-(3-amino-6-chloropyridazin-4-yl)-4-phenylpiperidin-4-yl)methyl)carbamate

4-bromo-6-chloropyridazin-3-amine (718 mg, 3.44 mmol, 1.0 eq) was combined with DMSO (6 mL). Tert-butyl ((4-phenylpiperidin-4-yl)methyl)carbamate (1 g, 3.44 mmol, 1.0 eq) and potassium carbonate (2.86 g, 20.7 mmol, 6.0 eq) were added. The reaction mixture was heated to 100° C. and stirred for 20 h under argon. The crude material was purified on silica column (heptane/EtOAc 0-100%) to afford the title compound (1.4 g, 3.18 mmol, 92% yield) as a grey solid. MS (ESI): 418.2011 ([M+H]⁺).

### b) tert-butyl ((1-(3-amino-6-(5-fluoro-2-hydroxyphenyl)pyridazin-4-yl)-4-phenylpiperidin-4-yl)methyl)carbamate

Tert-butyl ((1-(3-amino-6-chloropyridazin-4-yl)-4-phenylpiperidin-4-yl)methyl)carbamate (300 mg, 718 μmol, 1.0 eq), (5-fluoro-2-hydroxyphenyl)boronic acid (280 mg, 1.79 mmol, 2.5 eq) and K₂CO₃ (298 mg, 2.15 mmol, 3.0 eq), followed by methanesulfonato(2-dicyclohexylphosphino-2′,6′-di-1-propoxy-1,1′-biphenyl)(2′-amino-1,1′-biphenyl-2-yl)palladium(II) (RuPhos Pd G3) (18 mg, 21.5 μmol, 0.03 eq) were combined with degassed dioxane (10 mL)/water (1 mL). The reaction mixture was heated to 110° C. and stirred overnight. The crude material was purified on silica column (heptane/EtOAc 0-100%) as eluent to afford the title compound (120 mg, 224 μmol, 31% yield) as an off-white solid. MS (ESI): 494.2551 ([M+H]⁺).

### c) 2-(6-amino-5-(4-(aminomethyl)-4-phenylpiperidin-1-yl)pyridazin-3-yl)-4-fluorophenol

Tert-butyl ((1-(3-amino-6-(5-fluoro-2-hydroxyphenyl)pyridazin-4-yl)-4-phenylpiperidin-4-yl)methyl)carbamate (115 mg, 233 μmol, 1.0 eq) and 4N hydrogen chloride in dioxane (300 μL, 1.2 mmol, 5.15 eq) were combined with dioxane (1 mL). The reaction was stirred for 20 h. The precipitated solid was filtered off and dried under high vacuum to afford the title compound (75 mg, 167 μmol, 71% yield) as an off-white solid, hydrochloride salt. MS (ESI): 394.2027 ([M+H]⁺).

### d) N-((1-(3-amino-6-(5-fluoro-2-hydroxyphenyl)pyridazin-4-yl)-4-phenylpiperidin-4-yl)methyl)-4-(4-(4-((2,6-dioxopiperidin-3-yl)oxy)phenyl)piperidin-1-yl)butanamide 2,2,2-trifluoroacetate

2-(6-amino-5-(4-(aminomethyl)-4-phenylpiperidin-1-yl)pyridazin-3-yl)-4-fluorophenol, hydrochloride salt (20 mg, 46.5 μmol, 1.0 eq) and Ligase 39 (19.1 mg, 46.5 μmol, 1.0 eq) was stirred with DIPEA (15 mg, 20.3 μL, 116 μmol, 2.5 eq) in DMF (200 μL). HATU (21.2 mg, 55.8 μmol, 1.2 eq) was added and the mixture was stirred at room temperature overnight. The crude material was purified by prep-HPLC to afford the title compound (4 mg, 4.63 μmol, 9% yield) as a white solid, 2,2,2-trifluoroacetate salt. MS (ESI): 750.3761 ([M+H]⁺).

### Example 184

### N-[1-[3-amino-6-(2-hydroxyphenyl)pyridazin-4-yl]-4-phenyl-4-piperidyl]-2-[1-[1-[2-(2,6-dioxo-3-piperidyl)-1,3-dioxo-isoindolin-5-yl]piperidine-4-carbonyl]-4-piperidyl]acetamide

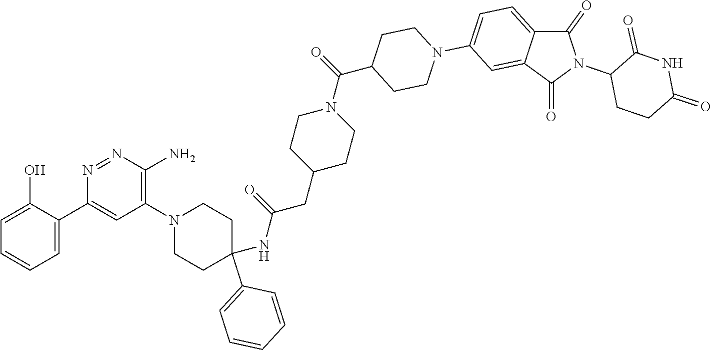 [SMILES: Nc1nnc(-c2ccccc2O)cc1N1CCC(NC(=O)CC2CCN(C(=O)C3CCN(c4ccc5c(c4)C(=O)N(C4CCC(=O)NC4=O)C5=O)CC3)CC2)(c2ccccc2)CC1]

### a) tert-butyl 4-(2-((1-(3-amino-6-(2-hydroxyphenyl)pyridazin-4-yl)-4-phenylpiperidin-4-yl)amino)-2-oxoethyl)piperidine-1-carboxylate

2-(6-amino-5-(4-amino-4-phenylpiperidin-1-yl)pyridazin-3-yl)phenol (400 mg, 1.11 mmol, 1.0 eq) was combined with 2-(1-(tert-butoxycarbonyl)piperidin-4-yl)acetic acid (296 mg, 1.22 mmol, 1.1 eq), HATU (505 mg, 1.33 mmol, 1.2 eq) and DIPEA (358 mg, 483 μL, 2.77 mmol, 2.5 eq) in DMF (4 mL). The reaction was purged with argon and stirred overnight. The solvent was evaporated. The mixture was extracted with EtOAc. The organic layer was washed with water and brine, dried over magnesium sulfate, filtered and evaporated. The crude residue was purified on silica column (heptane/EtOAc 50-100%) eluent to afford the title compound (571 mg, 934 μmol, 84% yield) as an orange solid. MS (ESI): 587.3335 ([M+H]⁺).

### b) N-(1-(3-amino-6-(2-hydroxyphenyl)pyridazin-4-yl)-4-phenylpiperidin-4-yl)-2-(piperidin-4-yl)acetamide

Tert-butyl 4-(2-((1-(3-amino-6-(2-hydroxyphenyl)pyridazin-4-yl)-4-phenylpiperidin-4-yl)amino)-2-oxoethyl)piperidine-1-carboxylate (565 mg, 963 μmol, 1.0 eq) was dissolved in DCM (5.6 mL) at room temperature. 4M HCl in dioxane (1.2 ml, 4.81 mmol, 5.0 eq) was added to the orange solution. The solution turned yellow and a yellow gum formed. Aqueous saturated NaHCO₃ was added until pH=10. The mixture was extracted with DCM and EtOAc. The organic layers were combined, dried over sodium sulfate, filtered and evaporated to afford the title compound (270 mg, 411 μmol, 42% yield) as a yellow solid. MS (ESI): 487.5 ([M+H]⁺).

### c) N-(1-(3-amino-6-(2-hydroxyphenyl)pyridazin-4-yl)-4-phenylpiperidin-4-yl)-2-(1-(1-(2-(2,6-dioxopiperidin-3-yl)-1,3-dioxoisoindolin-5-yl)piperidine-4-carbonyl)piperidin-4-yl)acetamide diformate

N-(1-(3-amino-6-(2-hydroxyphenyl)pyridazin-4-yl)-4-phenylpiperidin-4-yl)-2-(piperidin-4-yl)acetamide (25 mg, 51.4 μmol, 1.0 eq) was combined with Ligase 15 (19.8 mg, 51.4 μmol, 1.0 eq), HATU (23.4 mg, 61.6 μmol, 1.2 eq) and DIPEA (16.6 mg, 22.4 μL, 128 μmol, 2.5 eq) in DMF (250 μL) in a microwave flask. The reaction mixture was stirred for 2 h at room temperature. The crude material was purified by prep-HPLC to afford the title compound (24 mg, 20.3 μmol, 39% yield) as a yellow solid, diformate salt. MS (ESI): 854.3983 ([M+H]⁺).

### Example 185

### N-[1-[3-amino-6-(2-hydroxyphenyl)pyridazin-4-yl]-4-phenyl-4-piperidyl]-2-[1-[4-[4-[4-[(2,6-dioxo-3-piperidyl)amino]phenyl]-1-piperidyl]butanoyl]-4-piperidyl]acetamide

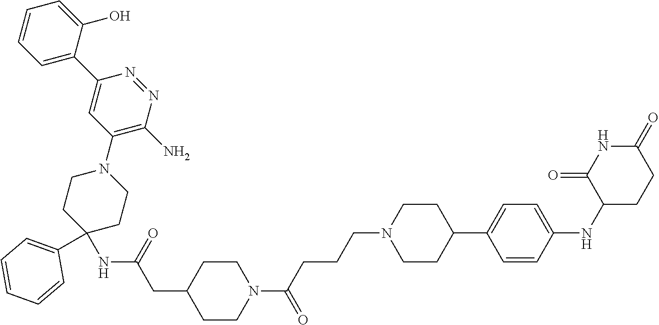 [SMILES: Nc1nnc(-c2ccccc2O)cc1N1CCC(NC(=O)CC2CCN(C(=O)CCCN3CCC(c4ccc(NC5CCC(=O)NC5=O)cc4)CC3)CC2)(c2ccccc2)CC1]

N-(1-(3-amino-6-(2-hydroxyphenyl)pyridazin-4-yl)-4-phenylpiperidin-4-yl)-2-(piperidin-4-yl)acetamide (25 mg, 51.4 μmol, 1.0 eq) was combined with Ligase 52 (21.1 mg, 51.4 μmol, 1.0 eq), HATU (23.4 mg, 61.6 μmol, 1.2 eq) and DIPEA (16.6 mg, 22.4 μL, 128 μmol, 2.5 eq) in DMF (250 μL) in a microwave flask. The reaction was stirred for 2 h at room temperature. The crude material was purified by prep-HPLC to afford the title compound (25 mg, 26 μmol, 50.5% yield) as a white solid, diformate salt. MS (ESI): 840.4566 ([M+H]⁺).

### Example 186

### 5-(4-(2-(1-(3-amino-6-(2-hydroxyphenyl)pyridazin-4-yl)-4-phenylpiperidine-4-carbonyl)-2,6-diazaspiro[3.4]octane-6-carbonyl)piperidin-1-yl)-2-(2,6-dioxopiperidin-3-yl)isoindoline-1,3-dione

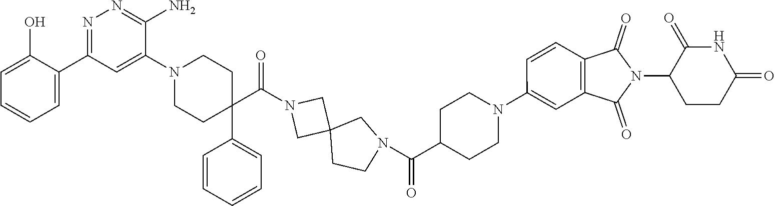 [SMILES: Nc1nnc(-c2ccccc2O)cc1N1CCC(C(=O)N2CC3(CCN(C(=O)C4CCN(c5ccc6c(c5)C(=O)N(C5CCC(=O)NC5=O)C6=O)CC4)C3)C2)(c2ccccc2)CC1]

### a) tert-butyl 2-[1-(3-amino-6-chloro-pyridazin-4-yl)-4-phenyl-piperidine-4-carbonyl]-2,7-diazaspiro[3.4]octane-7-carboxylate

To a solution of potassium 4-(3-amino-6-chloro-pyridazin-4-yl)-1-phenyl-piperazine-2-carboxylate (190 mg, 512 μmol, 1.0 eq), HATU (233 mg, 613 μmol 1.2 eq) and DIPEA (330 mg, 446 μL, 2.55 mmol, 5.0 eq) in DMF (3 mL) was added tert-butyl 1-oxa-4,9-diazaspiro[5.5]undecane-4-carboxylate (196 mg, 766 μmol, 1.5 eq). The reaction mixture was stirred at room temperature for 1 h. The reaction mixture was concentrated in vacuo.

The crude material was purified on silica column (DCM/MeOH 0-00%) to afford the title compound (221 mg, 420 μmol, 82% yield) as a light brown solid. MS (ESI): 527.3 ([M+H]⁺).

### b) tert-butyl 2-[1-[3-amino-6-(2-hydroxyphenyl)pyridazin-4-yl]-4-phenyl-piperidine-4-carbonyl]-2,7-diazaspiro[3.4]octane-7-carboxylate

A suspension of tert-butyl 2-(1-(3-amino-6-chloropyridazin-4-yl)-4-phenylpiperidine-4-carbonyl)-2,6-diazaspiro[3.4]octane-6-carboxylate (290 mg, 550 μmol, 1.0 eq), (2-hydroxyphenyl)boronic acid (190 mg, 1.38 mmol, 2.5 eq), potassium carbonate (228 mg, 1.65 mmol, 3.0 eq) and RuPhos Pd G3 (23.0 mg, 27.5 μmol, 0.05 eq) in a mixture of degassed dioxane (6 mL) and water (0.6 mL) was stirred at 110° C. for 2 h under argon. The reaction mixture was poured in saturated NaHCO₃ and extracted with EtOAc. The organic layers were combined, washed with water and brine. The organic layers were dried over Na₂SO₄ and concentrated in vacuo. The crude material was purified on silica column (heptane/EtOAc 0-100%) to afford the title compound (280 mg, 479 μmol, 87% yield) as a brown foam. MS (ESI): 585.4 ([M+H]⁺).

### c) [1-[3-amino-6-(2-hydroxyphenyl)pyridazin-4-yl]-4-phenyl-4-piperidyl]-(2,7-diazaspiro[3.4]octan-2-yl)methanone

A solution of tert-butyl 2-(1-(3-amino-6-(2-hydroxyphenyl)pyridazin-4-yl)-4-phenylpiperidine-4-carbonyl)-2,6-diazaspiro[3.4]octane-6-carboxylate (280 mg, 479 μmol, 1.0 eq) in 1,1,1,3,3,3-hexafluoro-2-propanol (4.02 g, 2.51 mL, 23.9 mmol, 50 eq) in a sealed tube was stirred at 120° C. for 72 h. The reaction mixture was cooled to room temperature and concentrated in vacuo to afford the title compound (160 mg, 330 μmol, 69% yield) as a light brown solid. MS (ESI): 485.3 ([M+H]⁺).

### d) 5-(4-(2-(1-(3-amino-6-(2-hydroxyphenyl)pyridazin-4-yl)-4-phenylpiperidine-4-carbonyl)-2,6-diazaspiro[3.4]octane-6-carbonyl)piperidin-1-yl)-2-(2,6-dioxopiperidin-3-yl)isoindoline-1,3-dione

Ligase 15 (19.1 mg, 49.5 μmol, 1.2 eq) was dissolved in dry DMF (400 μL). DIPEA (13.3 mg, 18 μL, 103 μmol, 2.5 eq) and HATU (20.4 mg, 53.7 μmol, 1.3 eq) were added and the mixture was stirred at room temperature for 10 min. (1-(3-amino-6-(2-hydroxyphenyl)pyridazin-4-yl)-4-phenylpiperidin-4-yl)(2,6-diazaspiro[3.4]octan-2-yl)methanone (20 mg, 41.3 μmol, 1.0 eq) was added and the mixture was stirred at room temperature for 2 h. The reaction mixture was purified directly by prep-HPLC to afford the title compound (12.2 mg, 13.6 μmol, 32% yield) as a yellow solid, formate salt. MS (ESI): 852.6 ([M+H]⁺).

### Example 187

### 5-(4-(2-(2-(1-(3-amino-6-(2-hydroxyphenyl)pyridazin-4-yl)-4-phenylpiperidine-4-carbonyl)-2,6-diazaspiro[3.4]octan-6-yl)-2-oxoethyl)piperidin-1-yl)-2-(2,6-dioxopiperidin-3-yl)isoindoline-1,3-dione

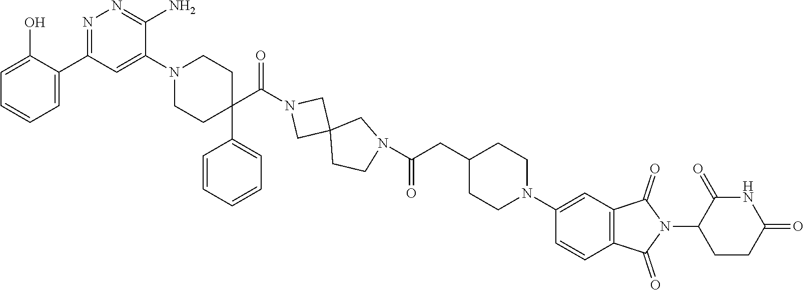 [SMILES: Nc1nnc(-c2ccccc2O)cc1N1CCC(C(=O)N2CC3(CCN(C(=O)CC4CCN(c5ccc6c(c5)C(=O)N(C5CCC(=O)NC5=O)C6=O)CC4)C3)C2)(c2ccccc2)CC1]

The title compound was prepared in analogy to example 186 step d using Ligase 24 as a yellow solid (14.8 mg, 16.2 μmol, 39% yield), formate salt. MS (ESI): 866.7 ([M+H]⁺).

### Example 188

### 5-(4-(3-(2-(1-(3-amino-6-(2-hydroxyphenyl)pyridazin-4-yl)-4-phenylpiperidine-4-carbonyl)-2,6-diazaspiro[3.4]octan-6-yl)-3-oxopropyl)piperidin-1-yl)-2-(2,6-dioxopiperidin-3-yl)isoindoline-1,3-dione

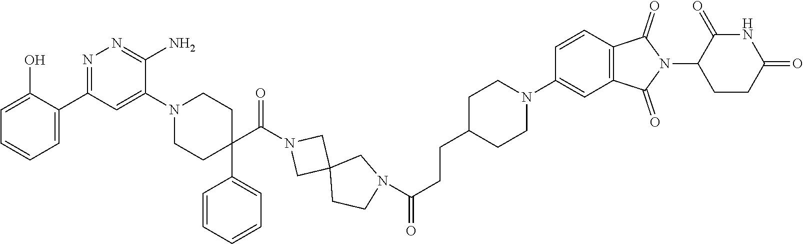 [SMILES: Nc1nnc(-c2ccccc2O)cc1N1CCC(C(=O)N2CC3(CCN(C(=O)CCC4CCN(c5ccc6c(c5)C(=O)N(C5CCC(=O)NC5=O)C6=O)CC4)C3)C2)(c2ccccc2)CC1]

The title compound was prepared in analogy to example 186 step d using Ligase 12 as a yellow solid (13.6 mg, 14.7 μmol, 35% yield), formate salt. MS (ESI): 880.7 ([M+H]⁺).

### Example 189

### 5-(2-(2-(1-(3-amino-6-(2-hydroxyphenyl)pyridazin-4-yl)-4-phenylpiperidine-4-carbonyl)-2,6-diazaspiro[3.4]octan-6-yl)-2-oxoethoxy)-2-(2,6-dioxopiperidin-3-yl)isoindoline-1,3-dione

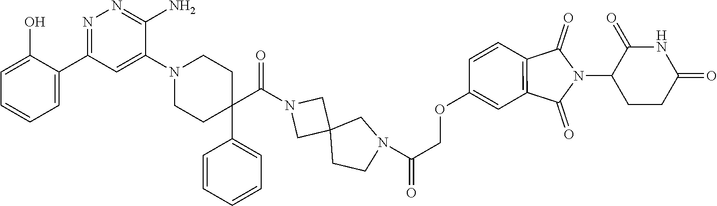 [SMILES: Nc1nnc(-c2ccccc2O)cc1N1CCC(C(=O)N2CC3(CCN(C(=O)COc4ccc5c(c4)C(=O)N(C4CCC(=O)NC4=O)C5=O)C3)C2)(c2ccccc2)CC1]

The title compound was prepared in analogy to example 186 step d using Ligase 48 as a yellow solid (10.5 mg, 12.4 μmol, 30% yield), formate salt. MS (ESI): 799.5 ([M+H]⁺).

### Example 190

### 5-(4-((2-(1-(3-amino-6-(2-hydroxyphenyl)pyridazin-4-yl)-4-phenylpiperidine-4-carbonyl)-2,6-diazaspiro[3.4]octan-6-yl)methyl)piperidin-1-yl)-2-(2,6-dioxopiperidin-3-yl)isoindoline-1,3-dione

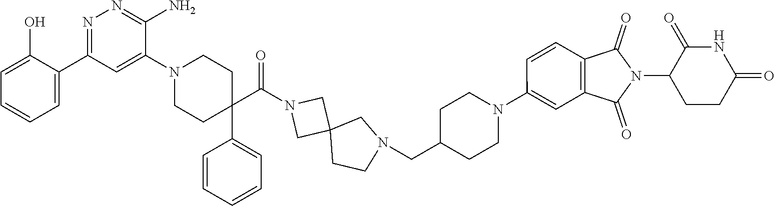 [SMILES: Nc1nnc(-c2ccccc2O)cc1N1CCC(C(=O)N2CC3(CCN(CC4CCN(c5ccc6c(c5)C(=O)N(C5CCC(=O)NC5=O)C6=O)CC4)C3)C2)(c2ccccc2)CC1]

(1-(3-amino-6-(2-hydroxyphenyl)pyridazin-4-yl)-4-phenylpiperidin-4-yl)(2,6-diazaspiro[3.4]octan-2-yl)methanone (20 mg, 41.3 μmol, 1.0 eq), Ligase 33 (35.8 mg, 82.5 μmol, 2.0 eq) and potassium iodide (6.85 mg, 41.3 μmol, 1.0 eq) were stirred at 70° C. in DMSO (400 μL) for 16 h. The reaction mixture was purified by prep-HPLC to afford the title compound (6.5 mg, 7.35 μmol, 17% yield) as a yellow solid, formate salt. MS (ESI): 838.5 ([M+H]⁺).

### Example 191

### 5-(4-(2-(2-(1-(3-amino-6-(2-hydroxyphenyl)pyridazin-4-yl)-4-phenylpiperidine-4-carbonyl)-2,6-diazaspiro[3.4]octan-6-yl)ethyl)piperidin-1-yl)-2-(2,6-dioxopiperidin-3-yl)isoindoline-1,3-dione

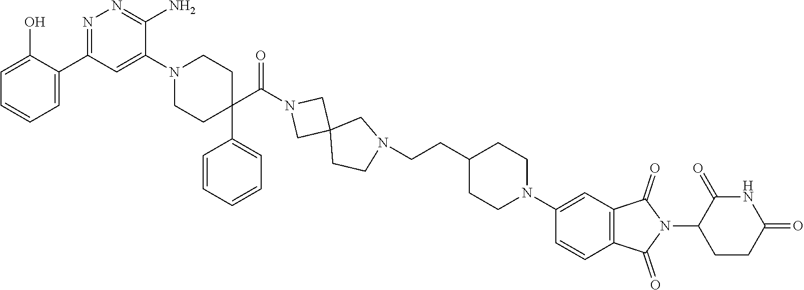 [SMILES: Nc1nnc(-c2ccccc2O)cc1N1CCC(C(=O)N2CC3(CCN(CCC4CCN(c5ccc6c(c5)C(=O)N(C5CCC(=O)NC5=O)C6=O)CC4)C3)C2)(c2ccccc2)CC1]

The title compound was prepared in analogy to example 190 using Ligase 25 as a yellow solid (9.6 mg, 10.7 μmol, 25% yield), formate salt. MS (ESI): 852.6 ([M+H]⁺).

### Example 192

### 5-[4-[2-[2-[1-[3-amino-6-(2-hydroxyphenyl)pyridazin-4-yl]-4-phenyl-piperidine-4-carbonyl]-2,7-diazaspiro[3.4]octan-7-yl]ethoxy]-1-piperidyl]-2-(2,6-dioxo-3-piperidyl)isoindoline-1,3-dione

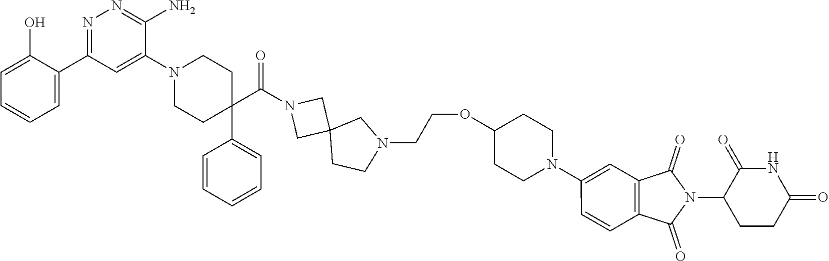 [SMILES: Nc1nnc(-c2ccccc2O)cc1N1CCC(C(=O)N2CC3(CCN(CCOC4CCN(c5ccc6c(c5)C(=O)N(C5CCC(=O)NC5=O)C6=O)CC4)C3)C2)(c2ccccc2)CC1]

The title compound was prepared in analogy to example 190 using Ligase 26 as a yellow solid (9.2 mg, 9.86 μmol, 23% yield), formate salt. MS (ESI): 868.6 ([M+H]⁺).

### Example 193

### rac-5-[4-[4-[[3-[3-[3-Amino-6-(2-hydroxyphenyl)pyridazin-4-yl]-3,8-diazabicyclo[3.2.1]octan-8-yl]-5-fluoro-phenyl]methyl]piperazine-1-carbonyl]-1-piperidyl]-2-(2,6-dioxo-3-piperidyl)isoindoline-1,3-dione

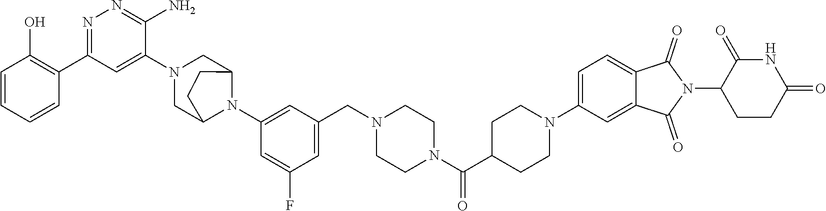 [SMILES: Nc1nnc(-c2ccccc2O)cc1N1CC2CCC(C1)N2c1cc(F)cc(CN2CCN(C(=O)C3CCN(c4ccc5c(c4)C(=O)N(C4CCC(=O)NC4=O)C5=O)CC3)CC2)c1]

### a) Benzyl 4-[(3-bromo-5-fluoro-phenyl)methyl]piperazine-1-carboxylate

To a solution of 1-bromo-3-(bromomethyl)-5-fluoro-benzene (0.4 g, 1.5 mmol, 1.0 eq) and triethylamine (0.23 g, 0.31 mL, 2.2 mmol, 1.5 eq) in THF (40 mL) was added benzyl piperazine-1-carboxylate (0.40 g, 0.35 mL, 1.8 mmol, 1.2 eq) and the reaction mixture was stirred at ambient temperature for 16 h. The reaction mixture was concentrated in vacuo, poured into water, extracted with EtOAc and washed with brine. The combined organic layer was dried over Na₂SO₄ and concentrated in vacuo to afford the title compound (0.63 g, 1.54 mmol, 99% yield) as a yellow oil. ¹H NMR (400 MHz, CDCl₃): δ=7.41-7.30 (m, 6H), 7.25-7.18 (m, 2H), 5.12 (s, 2H), 3.71 (br s, 6H), 2.84-2.39 (m, 4H). ¹³C NMR (100 MHz, CDCl₃): δ=164.0, 161.5, 155.0, 136.3, 128.6, 128.2, 128.0, 122.9, 120.5, 120.0, 67.5, 52.3, 52.2.

### b) tert-Butyl 8-[3-[(4-benzyloxycarbonylpiperazin-1-yl)methyl]-5-fluoro-phenyl]-3,8-diazabicyclo[3.2.1]octane-3-carboxylate

A solution of benzyl 4-[(3-bromo-5-fluoro-phenyl)methyl]piperazine-1-carboxylate (610 mg, 1.50 mmol, 1.0 eq), tert-butyl 3,8-diazabicyclo[3.2.1]octane-3-carboxylate (350 mg, 1.65 mmol, 1.1 eq), Pd(dba)₂ (86.1 mg, 150 μmol, 0.1 eq), 2-dicyclohexylphosphino-2′,6′-diisopropoxybiphenyl (69.9 mg, 150 μmol, 0.1 eq; RuPhos) and sodium tert-butoxide (360 mg, 3.74 mmol, 2.5 eq) in dioxane (10 mL) was degassed by purging with argon. The reaction mixture was heated by microwave irradiation to 100° C. for 2 h. The reaction mixture was filtered through celite, concentrated in vacuo and the crude material was purified by silica gel flash chromatography using heptane/EtOAc (1:1) as eluent to afford the title compound (680 mg, 1.26 mmol, 84% yield) as a yellow oil. ¹H NMR (400 MHz, CDCl₃): δ=7.39-7.28 (m, 5H), 6.51 (s, 1H), 6.45-6.41 (m, 1H), 6.36 (td, J=2.2, 11.8 Hz, 1H), 5.13 (s, 2H), 4.21-4.05 (m, 2H), 3.80-3.56 (m, 2H), 3.54-3.48 (m, 4H), 3.41 (s, 2H), 3.32-3.13 (m, 2H), 2.40 (br s, 4H), 2.04-1.98 (m, 2H), 1.84 (br dd, J=7.4, 14.7 Hz, 2H), 1.46-1.43 (m, 9H). ¹³C NMR (100 MHz, CDCl₃): δ=164.2 (d, J_{CF}=242.75 Hz), 156.1, 155.2, 147.8 (d, J_{CF}=10.40 Hz), 141.5 (d, J_{CF}=9.54 Hz), 136.7, 128.5, 128.0, 127.9, 111.3, 105.0 (d, J_{CF}=21.67 Hz), 101.2 (d, J_{CF}=26.01 Hz), 79.8, 67.1, 62.9, 54.8-54.2, 52.8, 46.5 (br s), 45.3 (br s), 43.8 (br s), 28.4, 27.0 (m).

### c) Benzyl 4-[[3-(3,8-diazabicyclo[3.2.1]octan-8-yl)-5-fluoro-phenyl]methyl]piperazine-1-carboxylate

Tert-Butyl 8-[3-[(4-benzyloxycarbonylpiperazin-1-yl)methyl]-5-fluoro-phenyl]-3,8-diazabicyclo[3.2.1]octane-3-carboxylate (650 mg, 1.21 mmol, 1.0 eq) was dissolved in DCM (10 mL) and TFA (5.4 g, 3.7 mL, 47.6 mmol, 39.3 eq) was slowly added. The reaction mixture was stirred at ambient temperature for 16 h. The reaction mixture was concentrated in vacuo to afford the title compound (903 mg, 1.35 mmol, 112% yield) as a brown solid, bis-(2,2,2-trifluoroacetic acid) salt. MS (ESI): 439.4 ([M+H]⁺).

### d) Benzyl 4-[[3-[3-(3-amino-6-chloro-pyridazin-4-yl)-3,8-diazabicyclo[3.2.1]octan-8-yl]-5-fluoro-phenyl]methyl]piperazine-1-carboxylate

To a solution of benzyl 4-[[3-(3,8-diazabicyclo[3.2.1]octan-8-yl)-5-fluoro-phenyl]methyl]piperazine-1-carboxylate; bis-(2,2,2-trifluoroacetic acid) salt (772 mg, 1.76 mmol, 1.0 eq) in DMSO (10 mL) was added 4-bromo-6-chloropyridazin-3-amine (404 mg, 1.94 mmol, 1.1 eq) and potassium carbonate (1.22 g, 8.8 mmol, 5.0 eq). The reaction mixture was heated to 110° C. for 18 h. The reaction mixture was poured into water, extracted with EtOAc, washed with brine and the organic layer were dried over MgSO₄ and concentrated in vacuo. The crude material was purified on silica column (DCM/MeOH 0-10%) to afford the title compound (493 mg, 0.87 mmol, 50% yield) as a brown solid. MS (ESI): 566.3 ([M+H]⁺).

### e) Benzyl 4-[[3-[3-[3-amino-6-(2-hydroxyphenyl)pyridazin-4-yl]-3,8-diazabicyclo[3.2.1]octan-8-yl]-5-fluoro-phenyl]methyl]piperazine-1-carboxylate

To a solution of benzyl 4-[[3-[3-(3-amino-6-chloro-pyridazin-4-yl)-3,8-diazabicyclo[3.2.1]octan-8-yl]-5-fluoro-phenyl]methyl]piperazine-1-carboxylate (422 mg, 745 μmol, 1.0 eq) in a mixture of dioxane (5 mL), DMA (0.25 mL) and water (0.5 mL) was added (2-hydroxyphenyl)boronic acid (257 mg, 1.86 mmol, 2.5 eq) and potassium carbonate (309 mg, 2.24 mmol, 3.0 eq). The solution was degassed by purging with argon and after addition of RuPhos Pd G3 (31.1 mg, 37.3 μmol, 0.05 eq) the reaction mixture was stirred at 90° C. for 5 h. The reaction mixture was poured into a saturated NH₄Cl solution, extracted with EtOAc, washed with brine and the organic layer was dried over MgSO₄ and concentrated in vacuo. The crude material was purified on silica column (EtOAc/iPrOH 0-50%) as eluent to afford the title compound (338 mg, 0.54 mmol, 73% yield) as a dark red solid. MS (ESI): 624.3085 ([M+H]⁺).

### f) 2-[6-Amino-5-[8-[3-fluoro-5-(piperazin-1-ylmethyl)phenyl]-3,8-diazabicyclo[3.2.1]octan-3-yl]pyridazin-3-yl]phenol

To a solution of benzyl 4-[[3-[3-[3-amino-6-(2-hydroxyphenyl)pyridazin-4-yl]-3,8-diazabicyclo[3.2.1]octan-8-yl]-5-fluoro-phenyl]methyl]piperazine-1-carboxylate (100 mg, 160 μmol, 1.0 eq) in DCM (2 mL) was added carefully HBr (393 mg, 264 μL, 1.6 mmol, 10.0 eq; 33% in acetic acid) and the reaction mixture was stirred at ambient temperature for 2 h. The reaction mixture was concentrated in vacuo to afford the title compound (75 mg, 131 μmol, 96% yield) as a yellow solid, hydrobromide salt. MS (ESI): 490.4 ([M+H]⁺).

### g) rac-5-[4-[4-[[3-[3-[3-Amino-6-(2-hydroxyphenyl)pyridazin-4-yl]-3,8-diazabicyclo[3.2.1]octan-8-yl]-5-fluoro-phenyl]methyl]piperazine-1-carbonyl]-1-piperidyl]-2-(2,6-dioxo-3-piperidyl)isoindoline-1,3-dione

2-[6-Amino-5-[8-[3-fluoro-5-(piperazin-1-ylmethyl)phenyl]-3,8-diazabicyclo[3.2.1]octan-3-yl]pyridazin-3-yl]phenol; hydrobromide salt (30 mg, 61.3 μmol, 1.0 eq) was combined with Ligase 15 (28.3 mg, 73.5 μmol, 1.2 eq), HATU (34.9 mg, 95 μmol, 1.5 eq) and DIPEA (39.6 mg, 54 μL, 306 μmol, 5.0 eq) in DMF (650 μL). The reaction mixture was stirred at ambient temperature for 8 h and was then purified by prep-HPLC to afford the title compound (5 mg, 5.15 μmol, 8% yield) as a light yellow solid, 2,2,2-trifluoroacetic acid salt. MS (ESI): 857.5 ([M+H]⁺).

### Example 194

### 2-[[1-[3-amino-6-(2-hydroxyphenyl)pyridazin-4-yl]-4-phenyl-4-piperidyl]oxy]-N-[1-[2-[4-[4-[(2,6-dioxo-3-piperidyl)oxy]phenyl]-1-piperidyl]acetyl]-4-piperidyl]acetamide

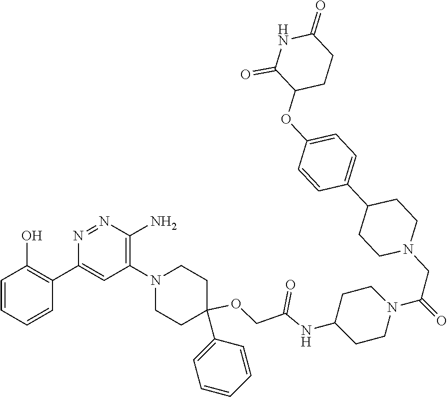 [SMILES: Nc1nnc(-c2ccccc2O)cc1N1CCC(OCC(=O)NC2CCN(C(=O)CN3CCC(c4ccc(OC5CCC(=O)NC5=O)cc4)CC3)CC2)(c2ccccc2)CC1]

### a) 2-((1-(tert-butoxycarbonyl)-4-phenylpiperidin-4-yl)oxy)acetic acid

Sodium hydride (865 mg, 21.6 mmol, 6.0 eq) was suspended in THF (2 mL) at room temperature. A clear solution of tert-butyl 4-hydroxy-4-phenylpiperidine-1-carboxylate (1 g, 3.61 mmol, 1.0 eq) in THF (5 mL) was added and the grey suspension was stirred for 1 h. Then a clear solution of 2-bromoacetic acid (1 g, 7.21 mmol, 2.0 eq) in THF (2 mL) was carefully added and the reaction was stirred overnight. Water was added to quench the reaction followed by aqueous HCl 1N until pH=4 and it was extracted with EtOAc. The organic layer was dried over magnesium sulfate, filtered and evaporated. The crude residue was purified on silica column (DCM/MeOH 0-20%) as eluent to afford the title compound (126 mg, 376 μmol, 10% yield) as a white solid. MS (ESI): 334.3 ([M+H]⁺).

### b) tert-butyl 4-(2-((1-benzylpiperidin-4-yl)amino)-2-oxoethoxy)-4-phenylpiperidine-1-carboxylate

2-((1-(tert-butoxycarbonyl)-4-phenylpiperidin-4-yl)oxy)acetic acid (160 mg, 477 μmol, 1.0 eq) was combined with 1-benzylpiperidin-4-amine (99.9 mg, 99.1 μL, 525 μmol, 1.1 eq), HATU (218 mg, 572 μmol, 1.2 eq) and DIPEA (154 mg, 208 μL, 1.19 mmol, 2.5 eq) in DMF (2.6 mL) at room temperature. The reaction mixture was stirred for 1 h. The reaction was extracted with water and EtOAc. The organic layer was washed with brine, dried over magnesium sulfate, filtered and evaporated. The crude residue was applied on silica gel and was purified on silica column (heptane/EtOAc 70-100%) to afford the title compound (290 mg, 286 μmol, 59% yield) as a colorless oil. MS (ESI): 508.9 ([M+H]⁺).

### c) N-(1-benzylpiperidin-4-yl)-2-((4-phenylpiperidin-4-yl)oxy)acetamide

TFA (651 mg, 440 μL, 5.71 mmol, 10.0 eq) was added to a solution of tert-butyl 4-(2-((1-benzylpiperidin-4-yl)amino)-2-oxoethoxy)-4-phenylpiperidine-1-carboxylate (290 mg, 571 μmol, 1.0 eq) in DCM (3 mL) at room temperature. The reaction was stirred overnight. Aqueous saturated NaHCO₃ was added slowly until pH=8 and the mixture was extracted with dichloromethane. The organic layer was dried over magnesium sulfate, filtered and evaporated to afford the title compound (157 mg, 385 μmol, 67% yield) as a light yellow oil. MS (ESI): 408.4 ([M+H]⁺).

### d) 2-((1-(3-amino-6-chloropyridazin-4-yl)-4-phenylpiperidin-4-yl)oxy)-N-(1-benzylpiperidin-4-yl)acetamide

N-(1-benzylpiperidin-4-yl)-2-((4-phenylpiperidin-4-yl)oxy)acetamide (155 mg, 380 μmol, 1.0 eq) was combined with 4-bromo-6-chloropyridazin-3-amine (87.2 mg, 418 μmol, 1.1 eq) and K₂CO₃ (131 mg, 951 μmol, 2.5 eq) in DMA (1.5 mL). The reaction mixture was heated to 120° C. and was stirred for 2 h. The solvent was evaporated. The crude residue was purified on silica column (DCM/MeOH 0-10%) as eluent to afford the title compound (88 mg, 163 μmol, 42% yield) as a light yellow solid. MS (ESI): 535.5 ([M+H]⁺).

### e) 2-((1-(3-amino-6-(2-hydroxyphenyl)pyridazin-4-yl)-4-phenylpiperidin-4-yl)oxy)-N-(1-benzylpiperidin-4-yl)acetamide

2-((1-(3-amino-6-chloropyridazin-4-yl)-4-phenylpiperidin-4-yl)oxy)-N-(1-benzylpiperidin-4-yl)acetamide (85 mg, 159 μmol, 1.0 eq) was combined with (2-hydroxyphenyl)boronic acid (43.8 mg, 318 μmol, 2.0 eq), RuPhos Pd G3 (6.64 mg, 7.94 μmol, 0.05 eq) and K₂CO₃ (54.9 mg, 397 μmol, 2.5 eq) in 1,4-dioxane (1 mL) and water (100 μL). The reaction was heated to 120° C. and was stirred for 2 h. The solvent was evaporated. The crude residue was purified on silica column (DCM/MeOH 0-10%) as eluent to afford the title compound (82 mg, 131 μmol, 82% yield) as a yellow oil. MS (ESI): 593.3233 ([M+H]⁺).

### f) 2-((1-(3-amino-6-(2-hydroxyphenyl)pyridazin-4-yl)-4-phenylpiperidin-4-yl)oxy)-N-(piperidin-4-yl)acetamide

Pd—C (14.4 mg, 13.5 μmol, 0.1 eq) was added to a solution of 2-((1-(3-amino-6-(2-hydroxyphenyl)pyridazin-4-yl)-4-phenylpiperidin-4-yl)oxy)-N-(1-benzylpiperidin-4-yl)acetamide (80 mg, 135 μmol, 1.0 eq) in MeOH (3.5 mL) at room temperature. The reaction mixture was purged with argon and was then stirred under hydrogen atmosphere overnight. The reaction was filtered through a filter, washed with DCM/methanol 9:1 and evaporated to afford the title compound (76 mg, 133 μmol, 98% yield) as a yellow solid. MS (ESI): 503.5 ([M+H]+).

### g) 2-((1-(3-amino-6-(2-hydroxyphenyl)pyridazin-4-yl)-4-phenylpiperidin-4-yl)oxy)-N-(1-(2-(4-(4-((2,6-dioxopiperidin-3-yl)oxy)phenyl)piperidin-1-yl)acetyl)piperidin-4-yl)acetamide diformate

2-((1-(3-amino-6-(2-hydroxyphenyl)pyridazin-4-yl)-4-phenylpiperidin-4-yl)oxy)-N-(piperidin-4-yl)acetamide (35 mg, 61.3 μmol, 1.0 eq) was combined with Ligase 35 (23.5 mg, 61.3 μmol, 1.0 eq), HATU (28 mg, 73.5 μmol, 1.2 eq) and DIPEA (19.8 mg, 26.8 μL, 153 μmol, 2.5 eq) in DMF (350 μL). The reaction was stirred at room temperature for 2 h. The reaction mixture was purified by prep-HPLC to afford the title compound (11 mg, 11.4 μmol, 18% yield) as a white solid, diformate salt. MS (ESI): 831.4174 ([M+H]⁺).

### Example 195

### rac-3-[4-[4-[2-[4-[[3-[3-[3-Amino-6-(2-hydroxyphenyl)pyridazin-4-yl]-3,8-diazabicyclo[3.2.1]octan-8-yl]phenyl]methyl]piperazin-1-yl]-2-oxo-ethyl]piperazin-1-yl]phenoxy]piperidine-2,6-dione

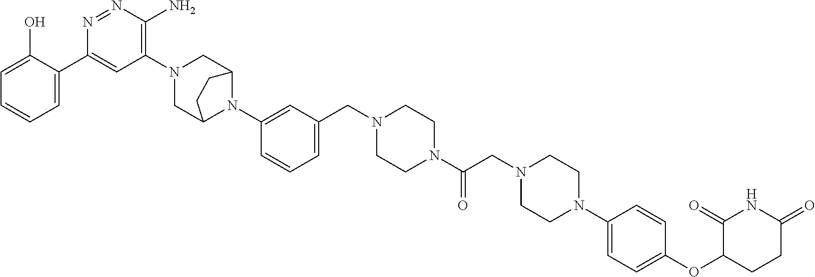 [SMILES: Nc1nnc(-c2ccccc2O)cc1N1CC2CCC(C1)N2c1cccc(CN2CCN(C(=O)CN3CCN(c4ccc(OC5CCC(=O)NC5=O)cc4)CC3)CC2)c1]

2-[6-Amino-5-[8-[3-(piperazin-1-ylmethyl)phenyl]-3,8-diazabicyclo[3.2.1]octan-3-yl]pyridazin-3-yl]phenol; hydrobromide salt (30.0 mg, 59 μmol, 1.0 eq; Example 46, Step f) was combined with Ligase 42 (27.2 mg, 59 μmol, 1.0 eq), HATU (44.9 mg, 118 μmol, 2.0 eq) and DIPEA (38.2 mg, 52 μL, 295 μmol, 5.0 eq) in DMF (650 μL). The reaction mixture was stirred at ambient temperature for 8 h and then purified by prep-HPLC to afford the title compound (8.7 mg, 21.8 μmol, 16% yield) as a light yellow solid, 2,2,2-trifluoroacetic acid salt. MS (ESI): 401.4 ([M+2H]²⁺).

### Example 196

### 2-[[1-[3-amino-6-(2-hydroxyphenyl)pyridazin-4-yl]-4-phenyl-4-piperidyl]oxy]-N-[1-[1-[2-(2,6-dioxo-3-piperidyl)-1,3-dioxo-isoindolin-5-yl]piperidine-4-carbonyl]-4-piperidyl]acetamide

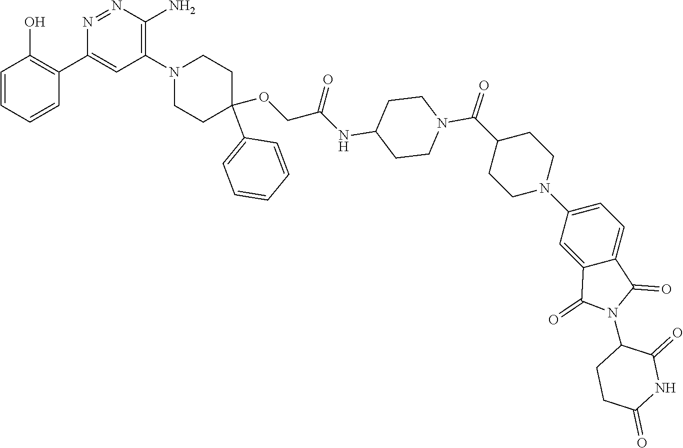 [SMILES: Nc1nnc(-c2ccccc2O)cc1N1CCC(OCC(=O)NC2CCN(C(=O)C3CCN(c4ccc5c(c4)C(=O)N(C4CCC(=O)NC4=O)C5=O)CC3)CC2)(c2ccccc2)CC1]

2-((1-(3-amino-6-(2-hydroxyphenyl)pyridazin-4-yl)-4-phenylpiperidin-4-yl)oxy)-N-(piperidin-4-yl)acetamide (35 mg, 61.3 μmol, 1.0 eq) was combined with Ligase 15 (23.6 mg, 61.3 μmol, 1.0 eq), HATU (28 mg, 73.5 μmol, 1.2 eq) and DIPEA (19.8 mg, 26.8 μL, 153 μmol, 2.5 eq) in DMF (350 μL). The reaction was stirred at room temperature for 2 h. The reaction mixture was purified by prep-HPLC to afford the title compound (3 mg, 2.53 μmol, 4% yield) as a yellow solid, diformate salt. MS (ESI): 870.3921 ([M+H]⁺).

### Example 197

### 5-[4-[4-[2-[3-[8-[3-amino-6-(2-hydroxyphenyl)pyridazin-4-yl]-5,8-diazaspiro[3.5]nonan-5-yl]phenoxy]ethyl]piperazine-1-carbonyl]-1-piperidyl]-2-(2,6-dioxo-3-piperidyl)isoindoline-1,3-dione

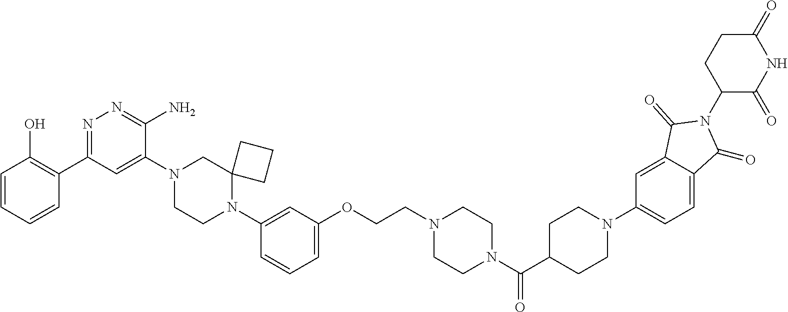 [SMILES: Nc1nnc(-c2ccccc2O)cc1N1CCN(c2cccc(OCCN3CCN(C(=O)C4CCN(c5ccc6c(c5)C(=O)N(C5CCC(=O)NC5=O)C6=O)CC4)CC3)c2)C2(CCC2)C1]

### a) tert-butyl 5-(3-(benzyloxy)phenyl)-5,8-diazaspiro[3.5]nonane-8-carboxylate

To a suspension of 1-(benzyloxy)-3-bromobenzene (1.2 g, 4.56 mmol, 1.0 eq) and tert-butyl 5,8-diazaspiro[3.5]nonane-8-carboxylate (1.08 g, 4.79 mmol, 1.05 eq) in t-BuOH (6.5 mL) was added K₂CO₃ (1.26 g, 9.12 mmol, 2.0 eq). The reaction was degassed with argon for 5 min. RuPhos Pd G3 (160 mg, 191 μmol, 0.0419 eq) was added. The reaction mixture was stirred at 120° C. overnight. The crude material was purified on silica column (heptane/EtOAc 0-50%) to afford the title compound (321 mg, 581 μmol, 12% yield) as a light yellow oil. MS (ESI): 409.2482 ([M+H]⁺).

### b) tert-butyl 5-(3-hydroxyphenyl)-5,8-diazaspiro[3.5]nonane-8-carboxylate

To a solution of tert-butyl 5-(3-(benzyloxy)phenyl)-5,8-diazaspiro[3.5]nonane-8-carboxylate (321 mg, 786 μmol, 1.0 eq) in methanol (10 mL) was added ammonium formate (991 mg, 15.7 mmol, 20 eq). The reaction mixture was degassed with argon for 10 min. Pd—C 10% (83.6 mg, 78.6 μmol, 0.1 eq) was added. The reaction mixture was stirred for 2 h at 70° C. The crude reaction mixture was purified on silica column (heptane/EtOAc 0-60%) to afford the title compound (200 mg, 628 μmol, 79% yield) as an off-white solid. MS (ESI): 317.1871 ([M−H]⁻).

### c) tert-butyl 5-(3-(2-(4-((benzyloxy)carbonyl)piperazin-1-yl)ethoxy)phenyl)-5,8-diazaspiro[3.5]nonane-8-carboxylate

To a solution of tert-butyl 5-(3-hydroxyphenyl)-5,8-diazaspiro[3.5]nonane-8-carboxylate (195 mg, 612 μmol, 1.0 eq) and benzyl 4-(2-hydroxyethyl)piperazine-1-carboxylate (CAS:14000-67-0, 173 mg, 655 μmol, 1.07 eq) in THF (1.5 mL) was added benzyl 4-(2-hydroxyethyl)piperazine-1-carboxylate (173 mg, 655 μmol, 1.07 eq). The reaction mixture was stirred at 70° C. for 2.5 h. 2-(trimethylphosphoranylidene)acetonitrile 0.5M in THF (0.75 mL, 375 μmol, 0.612 eq) was added. The reaction mixture was stirred for 1.5 h. Still some starting material remaining so 2-(trimethylphosphoranylidene)acetonitrile 0.5M in THF (0.75 mL, 375 μmol, 0.612 eq) was added again and the stirring was continued overnight at 70° C. The crude material was purified on silica column to afford the title compound (277 mg, 491 μmol, 80% yield) as a brown oil. MS (ESI): 565.3376 ([M+H]⁺).

### d) benzyl 4-(2-(3-(5,8-diazaspiro[3.5]nonan-5-yl)phenoxy)ethyl)piperazine-1-carboxylate

To a solution of tert-butyl 5-(3-(2-(4-((benzyloxy)carbonyl)piperazin-1-yl)ethoxy)phenyl)-5,8-diazaspiro[3.5]nonane-8-carboxylate (270 mg, 478 μmol, 1.0 eq) in dioxane (1 mL) was added 4M hydrogen chloride in dioxane (500 μL, 2 mmol, 4.18 eq). The reaction mixture was stirred overnight at room temperature. The precipitated material was filtered off and washed with diethyl ether and dried under high vacuum to afford the title compound (240 mg, 465 μmol, 97% yield) as a white solid, hydrochloride salt. MS (ESI): 465.2851 ([M+H]⁺).

### e) benzyl 4-(2-(3-(8-(3-amino-6-chloropyridazin-4-yl)-5,8-diazaspiro[3.5]nonan-5-yl)phenoxy)ethyl)piperazine-1-carboxylate

To a solution of benzyl 4-(2-(3-(5,8-diazaspiro[3.5]nonan-5-yl)phenoxy)ethyl)piperazine-1-carboxylate hydrochloride (235 mg, 469 μmol, 1.0 eq) and 4-bromo-6-chloropyridazin-3-amine (117 mg, 563 μmol, 1.2 eq) in DMSO (0.8 mL) was added K₂CO₃ (324 mg, 2.34 mmol, 5.0 eq). The reaction mixture was stirred for 16 h at 110° C. The reaction mixture was poured into THF/AcOEt 2:1 and washed with water/brine. The organic layer was dried over Na₂SO₄ and concentrated in vacuo. The crude material was purified on silica column (EtOAc/MeOH 0-5%) to afford the title compound (333 mg, 557 μmol, 119% yield) as a brown foam. MS (ESI): 592.2793 ([M+H]⁺).

### f) benzyl 4-(2-(3-(8-(3-amino-6-(2-hydroxyphenyl)pyridazin-4-yl)-5,8-diazaspiro[3.5]nonan-5-yl)phenoxy)ethyl)piperazine-1-carboxylate

To a suspension of benzyl 4-(2-(3-(8-(3-amino-6-chloropyridazin-4-yl)-5,8-diazaspiro[3.5]nonan-5-yl)phenoxy)ethyl)piperazine-1-carboxylate (330 mg, 446 μmol, 1.0 eq) and (2-hydroxyphenyl)boronic acid (92.2 mg, 669 μmol, 1.5 eq) in 1,4-dioxane (2 mL) and water (0.2 mL) was added K₂CO₃ (216 mg, 1.56 mmol, 3.5 eq) at room temperature. The reaction mixture was degassed with argon for 10 min. RuPhos Pd G3 (37.3 mg, 44.6 μmol, 0.1 eq) was added. The reaction mixture was heated at 90° C. for 2 h. The crude material was purified on silica column (heptane/EtOAc 0-100%) to afford the title compound (152 mg, 229 μmol, 51% yield) as a light yellow oil. MS (ESI): 650.3442 ([M+H]⁺).

### g) 2-(6-amino-5-(5-(3-(2-(piperazin-1-yl)ethoxy)phenyl)-5,8-diazaspiro[3.5]nonan-8-yl)pyridazin-3-yl)phenol

Benzyl 4-(2-(3-(8-(3-amino-6-(2-hydroxyphenyl)pyridazin-4-yl)-5,8-diazaspiro[3.5]nonan-5-yl)phenoxy)ethyl)piperazine-1-carboxylate (138 mg, 212 μmol, 1.0 eq) was stirred with palladium (22.6 mg, 21.2 μmol, 0.1 eq) in methanol (4 mL) and THF (2 mL) under a hydrogen atmosphere at room temperature overnight. The catalyst was filtered off, washed with diethyl ether and the solvent was evaporated under reduced pressure, then dried under high vacuum to afford the title compound (120 mg, 221 μmol, 104% yield) as a light yellow amorphous. MS (ESI): 516.3072 ([M+H]⁺).

### h) 5-(4-(4-(2-(3-(8-(3-amino-6-(2-hydroxyphenyl)pyridazin-4-yl)-5,8-diazaspiro[3.5]nonan-5-yl)phenoxy)ethyl)piperazine-1-carbonyl)piperidin-1-yl)-2-(2,6-dioxopiperidin-3-yl)isoindoline-1,3-dione diformate

2-(6-amino-5-(5-(3-(2-(piperazin-1-yl)ethoxy)phenyl)-5,8-diazaspiro[3.5]nonan-8-yl)pyridazin-3-yl)phenol (25 mg, 48.5 μmol, 1.0 eq) was combined with Ligase 15 (18.7 mg, 48.5 μmol, 1.0 eq), HATU (22.1 mg, 58.2 μmol, 1.2 eq) and DIPEA (18.8 mg, 25.4 μL, 145 μmol, 3.0 eq) in DMF (300 μL) at room temperature. The reaction was stirred overnight at room temperature. The reaction mixture was purified by prep-HPLC to afford the title compound (21 mg, 21.5 μmol, 44% yield) as a yellow salt, diformate salt. MS (ESI): 881.4127 ([M−H]⁻).

### Example 198

### 3-[[6-[1-[2-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-2-oxo-ethyl]-4-piperidyl]-3-pyridyl]oxy]piperidine-2,6-dione

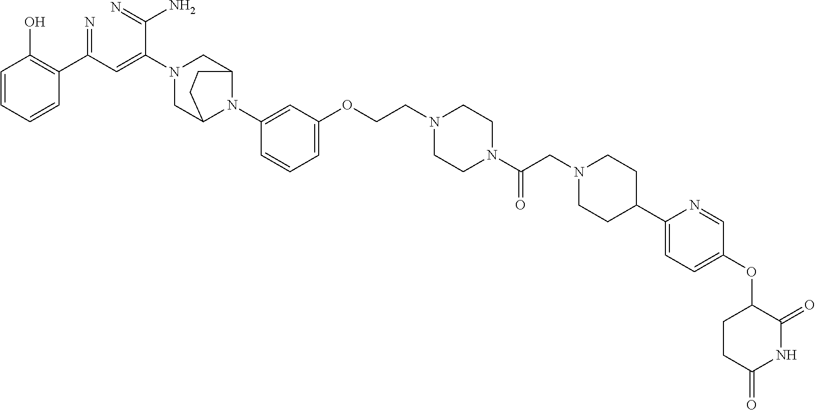 [SMILES: N=C(N)/C(=C\C(=N)c1ccccc1O)N1CC2CCC(C1)N2c1cccc(OCCN2CCN(C(=O)CN3CCC(c4ccc(OC5CCC(=O)NC5=O)cn4)CC3)CC2)c1]

2-[6-amino-5-[8-[3-(2-piperazin-1-ylethoxy)phenyl]-3,8-diazabicyclo[3.2.1]octan-3-yl]pyridazin-3-yl]phenol (20 mg, 39.9 μmol, 1.0 eq) was combined with Ligase 72 (15.3 mg, 39.9 μmol, 1.0 eq), HATU (18.9 mg, 49.8 μmol, 1.25 eq) and DIPEA (15.5 mg, 20.9 μL, 120 μmol, 3.0 eq) in DMF (200 μL) at room temperature. The reaction was stirred overnight at room temperature. The crude was purified by prep-HPLC to afford the title compound (15 mg, 16.9 μmol, 42% yield) as an off-white solid, formate salt. MS (ESI): 831.4294 ([M+H]⁺).

### Example 199

### 2-(2,6-dioxo-3-piperidyl)-4-[1-[1-[1-[5-[4-[4-[rac-(3S)-3-[3-amino-6-(2-hydroxyphenyl)pyridazin-4-yl]oxy-1-piperidyl]phenyl]piperazin-1-yl]pentanoyl]azetidin-3-yl]triazol-4-yl]ethoxy]isoindoline-1,3-dione

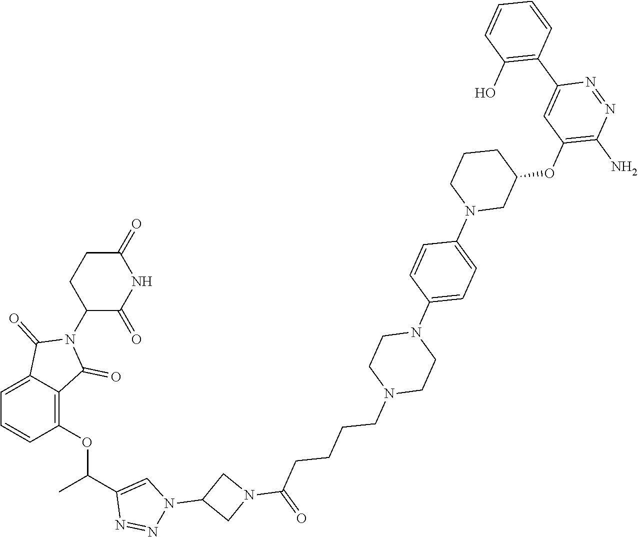 [SMILES: CC(Oc1cccc2c1C(=O)N(C1CCC(=O)NC1=O)C2=O)c1cn(C2CN(C(=O)CCCCN3CCN(c4ccc(N5CCC[C@H](Oc6cc(-c7ccccc7O)nnc6N)C5)cc4)CC3)C2)nn1]

To a solution of 5-[4-[4-[(3S)-3-[3-amino-6-(2-hydroxyphenyl)pyridazin-4-yl]oxy-1-piperidyl]phenyl]piperazin-1-yl]pentanoic acid (60.0 mg, 90.9 μmol, TFA salt) and Ligase 73 (48.8 mg, 90.9 μmol, TFA) in DMF (2.0 mL) was added (1-cyano-2-ethoxy-2-oxoethylidenaminooxy)dimethylamino-morpholino-carbenium hexafluorophosphate (COMU) (58.4 mg, 136.4 μmol) followed by DIPEA (94.0 mg, 727 μmol, 126 μL) at room temperature under nitrogen atmosphere and the reaction mixture was stirred at room temperature for 3 h. The reaction mixture was diluted with water and the solid precipitated out was filtered, washed with water, and diethyl ether. The crude residue was purified by prep-HPLC to afford the title compound (31.0 mg, 31.2 μmol, 34%) as an off-white solid, trifluoroacetic acid salt. MS (ESI): 954.3 ([M+H]+).

### Example 200

### 2-(2,6-dioxo-3-piperidyl)-4-[1-[1-[3-[4-[3-[4-[4-[rac-(3S)-3-[3-amino-6-(2-hydroxyphenyl)pyridazin-4-yl]oxy-1-piperidyl]phenyl]piperazin-1-yl]propanoyl]piperazin-1-yl]propyl]triazol-4-yl]ethoxy]isoindoline-1,3-dione

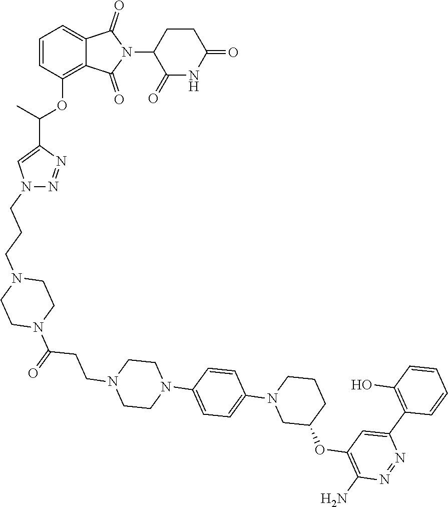 [SMILES: CC(Oc1cccc2c1C(=O)N(C1CCC(=O)NC1=O)C2=O)c1cn(CCCN2CCN(C(=O)CCN3CCN(c4ccc(N5CCC[C@H](Oc6cc(-c7ccccc7O)nnc6N)C5)cc4)CC3)CC2)nn1]

To a solution of 3-[4-[4-[(3S)-3-[3-amino-6-(2-hydroxyphenyl)pyridazin-4-yl]oxy-1-piperidyl]phenyl]piperazin-1-yl]propanoic acid (51.9 mg, 82.1 μmol, TFA salt) and Ligase 74 (50.0 mg, 82.1 μmol, TFA salt) in DMF (0.8 mL) was added (1-cyano-2-ethoxy-2-oxoethylidenaminooxy)dimethylamino-morpholino-carbenium hexafluorophosphate (COMU) (52.7 mg, 123 μmol) followed by DIPEA (84.9 mg, 657 μmol, 114 μL) at room temperature under nitrogen atmosphere and the reaction mixture was stirred at room temperature for 16 h. The reaction mixture was diluted with water and the solid precipitated out was filtered, washed with water, and diethyl ether. The crude residue was purified by prep-HPLC to afford the title compound (6.3 mg, 5.55 μmol, 6% yield) as a light brown solid, trifluoroacetic acid salt. MS (ESI): 996.3 ([M+H]+).

### Example 201

### 2-(2,6-dioxo-3-piperidyl)-4-[1-[1-[9-[4-[4-[rac-(3S)-3-[3-amino-6-(2-hydroxyphenyl)pyridazin-4-yl]oxy-1-piperidyl]phenyl]piperazin-1-yl]nonyl]triazol-4-yl]ethylamino]isoindoline-1,3-dione

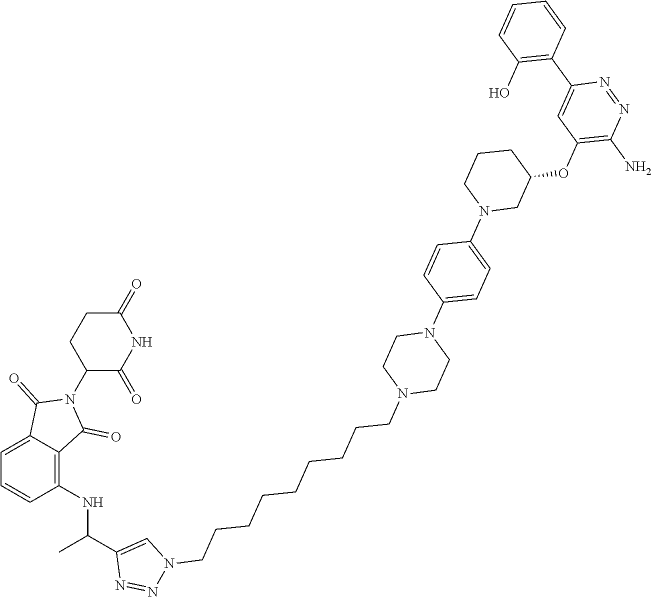 [SMILES: CC(Nc1cccc2c1C(=O)N(C1CCC(=O)NC1=O)C2=O)c1cn(CCCCCCCCCN2CCN(c3ccc(N4CCC[C@H](Oc5cc(-c6ccccc6O)nnc5N)C4)cc3)CC2)nn1]

To a solution of Ligase 30 (50.0 mg, 98.3 μmol) and 2-[6-amino-5-[[(3S)-1-(4-piperazin-1-ylphenyl)-3-piperidyl]oxy]pyridazin-3-yl]phenol (55.0 mg, 98.3 μmol, TFA salt) in tetrahydrofuran (2.0 mL) was added dibutyltin dichloride (29.8 mg, 98.3 μmol, 21.9 μL) followed by phenylsilane (12.7 mg, 117 μmol) at room temperature under nitrogen atmosphere and the reaction mixture was heated at 80° C. for 16 h. Reaction mixture was cooled to room temperature, diluted with water and extracted with ethyl acetate. The organic layer was washed with water, brine solution, dried over anhydrous sodium sulfate, filtered and concentrated under reduced pressure. The crude residue was purified by prep-HPLC to afford the title compound (8.7 mg, 8.70 μmol, 8% yield) as a light yellow solid, trifluoroacetic acid salt. MS (ESI): 939.3 ([M+H]⁺).

### Example 202

### 2-(2,6-dioxo-3-piperidyl)-4-[1-[1-[6-[2-[4-[4-[rac-(3S)-3-[3-amino-6-(2-hydroxyphenyl)pyridazin-4-yl]oxy-1-piperidyl]phenyl]piperazin-1-yl]ethoxy]hexyl]triazol-4-yl]ethoxy]isoindoline-1,3-dione

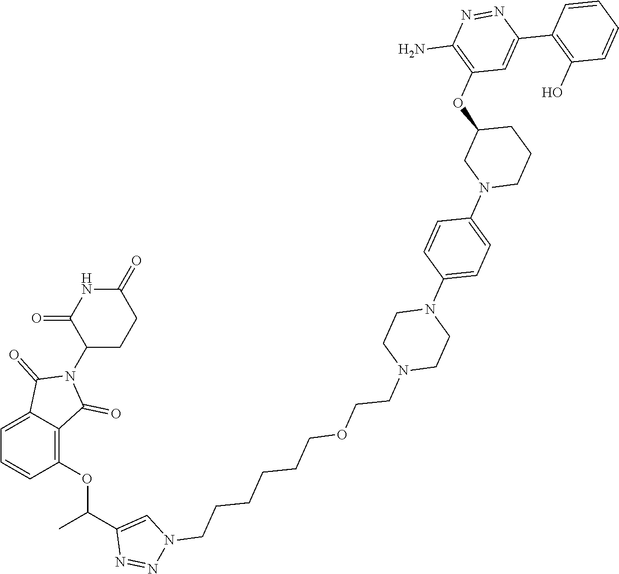 [SMILES: CC(Oc1cccc2c1C(=O)N(C1CCC(=O)NC1=O)C2=O)c1cn(CCCCCCOCCN2CCN(c3ccc(N4CCC[C@H](Oc5cc(-c6ccccc6O)nnc5N)C4)cc3)CC2)nn1]

To a solution of Ligase 76 (15.90 mg, 48.7 μmol) and 2-[6-amino-5-[[(3S)-1-[4-[4-[2-(6-azidohexoxy)ethyl]piperazin-1-yl]phenyl]-3-piperidyl]oxy]pyridazin-3-yl]phenol (30 mg, 48.7 μmol) in dimethyl sulfoxide (1 mL)/water (0.5 mL) was added sodium ascorbate (2.90 mg, 14.6 μmol) followed by copper sulfate (777.6 ug, 4.87 μmol, 2.16e-1 μL) at room temperature and the reaction mixture was stirred at room temperature for 5 h. The reaction mixture was diluted with water and extracted with dichloromethane. The organic layer was washed with water, brine solution, dried over anhydrous sodium sulfate, filtered and concentrated under reduced pressure. The crude residue was purified by prep-HPLC to afford the title compound (4.72 mg, 4.69 μmol, 9% yield) as an off-white solid, trifluoroacetic acid salt. MS (ESI): 942.3 ([M+H]⁺).

### Example 203

### 2-(2,6-dioxo-3-piperidyl)-4-[1-[1-[9-[4-[4-[rac-(3S)-3-[3-amino-6-(2-hydroxyphenyl)pyridazin-4-yl]oxy-1-piperidyl]phenyl]piperazin-1-yl]nonyl]triazol-4-yl]propylamino]isoindoline-1,3-dione

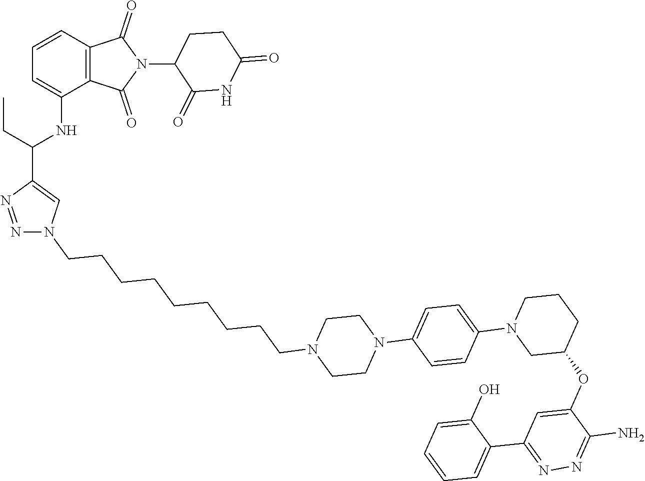 [SMILES: CCC(Nc1cccc2c1C(=O)N(C1CCC(=O)NC1=O)C2=O)c1cn(CCCCCCCCCN2CCN(c3ccc(N4CCC[C@H](Oc5cc(-c6ccccc6O)nnc5N)C4)cc3)CC2)nn1]

To a solution of Ligase 32 (46.8 mg, 89.5 μmol) and 2-[6-amino-5-[[(3S)-1-(4-piperazin-1-ylphenyl)-3-piperidyl]oxy]pyridazin-3-yl]phenol (0.04 g, 89.5 μmol) in tetrahydrofuran (4 mL) was added dibutyltin dichloride (27.2 mg, 89.5 μmol, 20.0 μL) followed by phenylsilane (11.6 mg, 107 μmol) at room temperature under nitrogen atmosphere and the reaction mixture was heated at 80° C. for 16 h. Reaction mixture was cooled to room temperature, diluted with water and extracted with ethyl acetate. The organic layer was washed with water, brine solution, dried over anhydrous sodium sulfate, filtered and concentrated under reduced pressure. The crude residue was purified by prep-HPLC to afford the title compound (2.6 mg, 2.40 μmol, 2% yield) as a light yellow solid, trifluoroacetic acid salt. MS (ESI): 953.6 ([M+H]⁺).

### Example 204

### 2-(2,6-dioxo-3-piperidyl)-4-[1-[1-[3-oxo-3-[4-[3-[4-[4-[rac-(3S)-3-[3-amino-6- (2-hydroxyphenyl)pyridazin-4-yl]oxy-1-piperidyl]phenyl]piperazin-1-yl]propyl]piperazin-1-yl]propyl]triazol-4-yl]ethoxy]isoindoline-1,3-dione

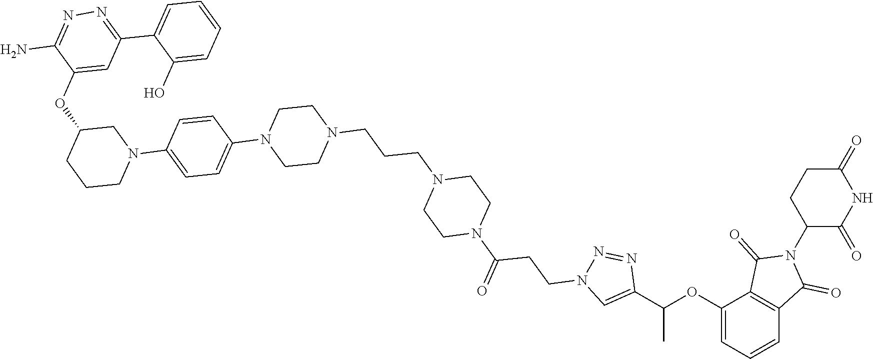 [SMILES: CC(Oc1cccc2c1C(=O)N(C1CCC(=O)NC1=O)C2=O)c1cn(CCC(=O)N2CCN(CCCN3CCN(c4ccc(N5CCC[C@H](Oc6cc(-c7ccccc7O)nnc6N)C5)cc4)CC3)CC2)nn1]

To a solution of 2-[6-amino-5-[[(3S)-1-[4-[4-(3-piperazin-1-ylpropyl)piperazin-1-yl]phenyl]-3-piperidyl]oxy]pyridazin-3-yl]phenol (55 mg, 96.0 μmol) and Ligase 75 (42.39 mg, 96.0 μmol) in DMF (1.5 mL) was added (1-cyano-2-ethoxy-2-oxoethylidenaminooxy)dimethylamino-morpholino-carbenium hexafluorophosphate (COMU) (61.6 mg, 144 μmol) followed by DIPEA (62.0 mg, 480 μmol, 83.6 μL) at room temperature under nitrogen atmosphere and the reaction mixture was stirred at room temperature for 16 h. The reaction mixture was diluted with water and extracted with dichloromethane. The organic layer was washed with water, brine solution, dried over anhydrous sodium sulfate, filtered and concentrated under reduced pressure. The crude residue was purified by prep-HPLC to afford the title compound (9.28 mg, 8.09 μmol, 8% yield) as an off-white solid, trifluoroacetic acid salt. MS (ESI): 996.3 ([M+H]⁺).

1. A compound of formula (I) 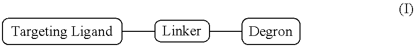
or a pharmaceutically acceptable salt thereof, wherein:
said targeting ligand is of formula (TL): 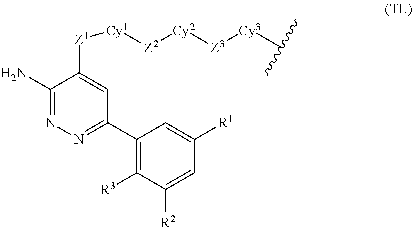
wherein:
R¹ and R² are each independently selected from the group consisting of hydrogen and halogen;
R³ is selected from the group consisting of amino and hydroxy;
Z¹ is:
(i) absent;
(ii) —O—; or
(iii) —O—C₁-C₆-alkyldiyl-CH(C₆-C₁₀-aryl)-C₁-C₆-alkyldiyl-NHC(O)—;
Cy¹ is 3-14 membered heterocyclyl optionally substituted with 1-3 substituents R⁴;
Z² is:
(i) absent;
(ii) carbonyl;
(iii) —NH—;
(iv) —C₁-C₆-alkyldiyl-;
(v) —C₁-C₆-alkyldiyl-NH—;
(vi) —O(CH₂)ₐ—;
(vii) —C(O)NH(CH₂)_{b}—;
(viii) —(CH₂)_{c}NHC(O)(CH₂)_{d}X¹—;
(ix) —O—C₁-C₆-alkyldiyl-C(O)—;
(x) —O—C₁-C₆-alkyldiyl-C(O)NH—; or
(xi) —CH₂N(C₁-C₆-alkyl)CH₂—;
Cy² is:
(i) absent;
(ii) C₆-C₁₀-aryl optionally substituted with 1-3 substituents R⁵;
(iii) C₃-C₁₀-cycloalkyl optionally substituted with 1-3 substituents R⁶;
(iv) 3-14 membered heterocyclyl optionally substituted with 1-3 substituents R⁷; or
(v) 5-14 membered heteroaryl optionally substituted with 1-3 substituents R⁸;
Z³ is:
(i) absent;
(ii) —X²(CH₂)ₑ—; or
(iii) —(CH₂)ₑX²—;
Cy³ is:
(i) absent;
(ii) C₆-C₁₀-aryl optionally substituted with 1-3 substituents R⁹;
(iii) C₃-C₁₀-cycloalkyl optionally substituted with 1-3 substituents R¹⁰; or
(iv) 3-14 membered heterocyclyl optionally substituted with 1-3 substituents R¹¹;
a, b, c, d, and e are each independently an integer selected from 0, 1, 2, 3, 4, 5, and 6;
X¹ is:
(i) absent;
(ii) —NH—; or
(iii) —O—;
X² is:
(i) absent;
(ii) carbonyl;
(iii) —O—; or
(iv) —NHC(O)—;
R⁴, R⁵, R⁶, R⁷, R⁸, R⁹, R¹⁰, and R¹¹ are each independently selected from the group consisting of hydroxy, amino, cyano, halogen, C₁-C₆-alkyl, C₁-C₆-alkoxy, halo-C₁-C₆-alkyl, halo-C₁-C₆-alkoxy, amino-C₁-C₆-alkyl, (C₁-C₆-alkyl)₂N—C₁-C₆-alkyl-, (C₁-C₆-alkyl)₂N—C₁-C₆-alkoxy-, C₁-C₆-alkyl-NH—C₁-C₆-alkyl-, C₁-C₆-alkyl-NH—C(O)—, C₁-C₆-alkyl-C(O)—NH—, 3-14 membered heterocyclyl, 3-14 membered heterocyclyloxy, 3-14 membered heterocyclyl-C₁-C₆-alkyl, 3-14 membered heterocyclyl-C₁-C₆-alkoxy and C₆-C₁₀-aryl; and
the wavy line indicates the point of attachment to the linker;
said linker is a covalent bond or is selected from the group consisting of formulae L-1 to L-23; 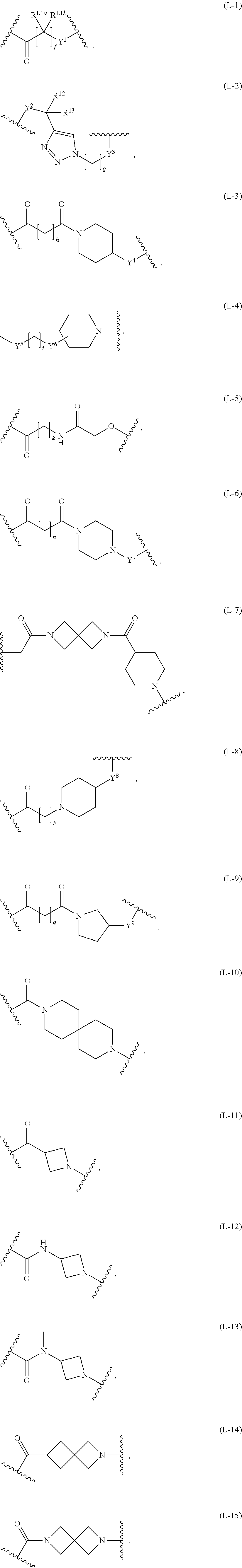 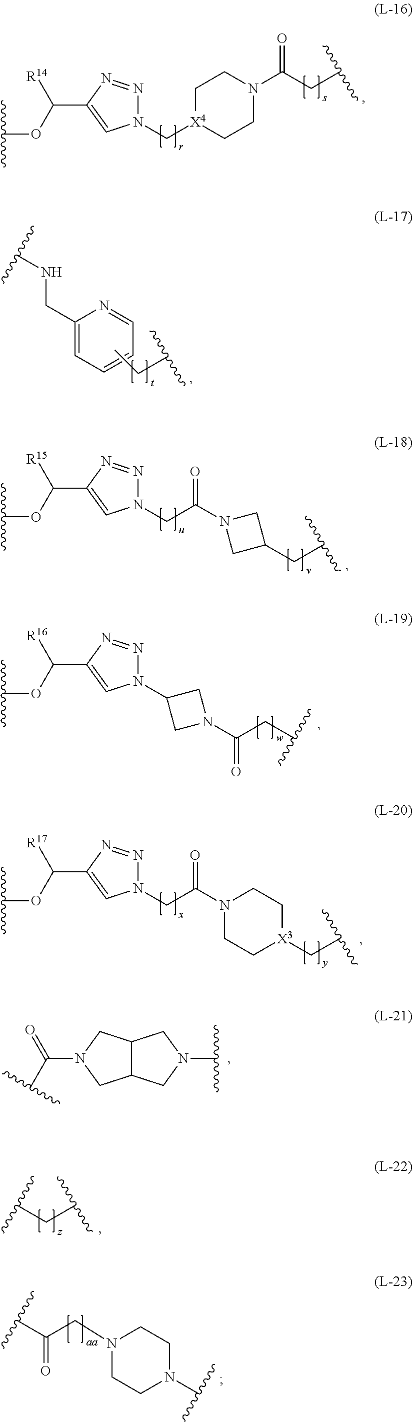
wherein:
X³ and X⁴ are independently selected from the group consisting of CH and N;
R¹² and R¹³ are independently selected from the group consisting of hydrogen and C₁-C₆-alkyl; or
R¹² and R¹³, taken together with the carbon atom to which they are attached, form a C₃-C₁₀-cycloalkyl ring;
R¹⁴, R¹⁵, R¹⁶, and R¹⁷ are independently selected from the group consisting of hydrogen and C₁-C₆-alkyl;
R^{L1a} and R^{L1b} are each independently selected from the group consisting of hydrogen, C₁-C₆-alkyl, halo-C₁-C₆-alkyl, C₁-C₆-alkoxy, halo-C₁-C₆-alkoxy, C₃-C₁₀-cycloalkyl, 3-14 membered heterocyclyl, C₆-C₁₀-aryl and 5-14 membered heteroaryl;
f, g, h, i, k, m, n, p, q, r, s, t, u, v, w, x, y, z, and aa are each independently an integer selected from 0, 1, 2, 3, 4, 5, 6, 7, 8, 9, 10, 11, 12, 13, and 14;
Y¹, Y², Y³, Y⁴, Y⁵, Y⁶, Y⁷, Y⁸, and Y⁹ are each independently absent or selected from the group consisting of —O—, —NH—, —N(C₁-C₆-alkyl)-, —C₁-C₆-alkyldiyl-, —NH—C₁-C₆-alkyldiyl-, —O—C₁-C₆-alkyldiyl-, carbonyl, —NHC(O)—, —N(C₁-C₆-alkyl)-C(O)—, —C(O)—N(C₁-C₆-alkyl)-, and —C(O)NH—;
each occurrence of a wavy line indicates the point of attachment of the linker to the targeting ligand or to the degron; and
said degron is selected from the group consisting of formulae (DG-1), (DG-2), (DG-3) and (DG-4): 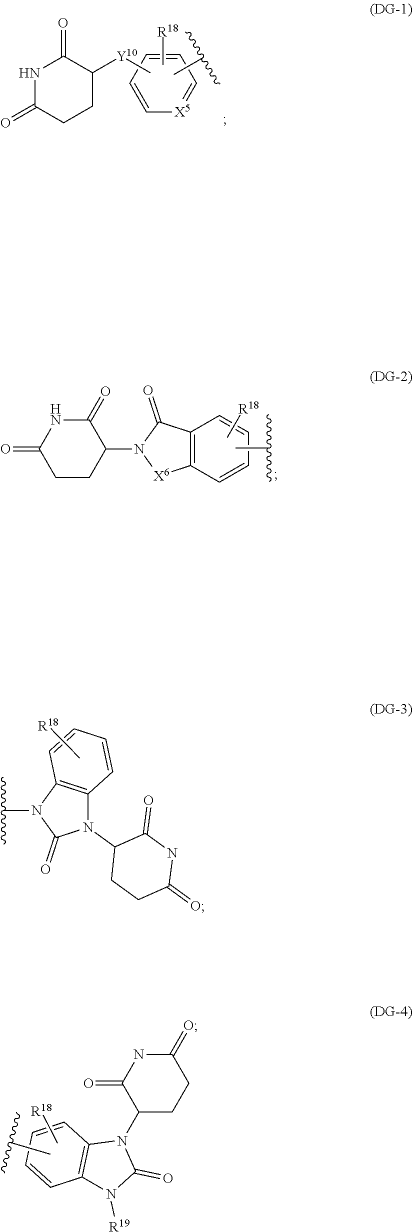
wherein:
X⁵ is CH or N;
X⁶ is CH₂ or C(O);
each R¹⁸ is independently selected from the group consisting of hydrogen, halogen and C₁-C₆-alkyl;
R¹⁹ is selected from the group consisting of hydrogen and C₁-C₆-alkyl;
Y¹⁰ is a covalent bond, —O— or —NR—, wherein R is selected from the group consisting of hydrogen, C₁-C₆-alkyl, halo-C₁-C₆-alkyl, C₃-C₁₀-cycloalkyl, 3-14 membered heterocyclyl, C₆-C₁₀-aryl and 5-14 membered heteroaryl; and
the wavy line indicates the point of attachment to the linker.

2. The compound of formula (I) according to claim 1, or a pharmaceutically acceptable salt thereof, wherein said targeting ligand is of formula (TL), wherein:
R¹ and R² are each independently selected from the group consisting of hydrogen and halogen;
R³ is selected from the group consisting of amino and hydroxy;
Z¹ is:
(i) absent;
(ii) —O—; or
(iii) —O—C₁-C₆-alkyldiyl-CH(C₆-C₁₀-aryl)-C₁-C₆-alkyldiyl-NHC(O)—;
Cy¹ is 3-14 membered heterocyclyl optionally substituted with R⁴;
Z² is:
(i) absent;
(ii) carbonyl;
(iii) —O(CH₂)ₐ—;
(iv) —C(O)NH(CH₂)_{b}—;
(v) —(CH₂)_{c}NHC(O)(CH₂)_{d}X¹—;
(vi) —O—C₁-C₆-alkyldiyl-C(O)NH—; or
(vii) —CH₂N(C₁-C₆-alkyl)CH₂—;
Cy² is:
(i) absent;
(ii) C₆-C₁₀-aryl optionally substituted with R⁵;
(iii) C₃-C₁₀-cycloalkyl;
(iv) 3-14 membered heterocyclyl; or
(v) 5-14 membered heteroaryl;
Z³ is:
(i) absent;
(ii) —X²(CH₂)ₑ—; or
(iii) —(CH₂)ₑX²;
Cy³ is:
(i) absent;
(ii) C₆-C₁₀-aryl;
(iii) C₃-C₁₀-cycloalkyl; or
(iv) 3-14 membered heterocyclyl;
a is 0, 1 or 2;
b is 0 or 1;
c, d, and e are each independently an integer selected from 0, 1, 2 and 3;
X¹ is:
(i) absent;
(ii) —NH—; or
(iii) —O—;
X² is:
(i) absent;
(ii) carbonyl;
(iii) —O—; or
(iv) —NHC(O)—;
R⁴ is C₆-C₁₀-aryl;
R⁵ is selected from the group consisting of halogen, C₁-C₆-alkyl, and halo-C₁-C₆-alkyl; and
the wavy line indicates the point of attachment to the linker.

3. The compound of formula (I) according to claim 1, or a pharmaceutically acceptable salt thereof, wherein said targeting ligand is of formula (TL), wherein:
R¹ is selected from the group consisting of hydrogen and halogen;
R² is hydrogen;
R³ is hydroxy;
Z¹ is absent;
Cy¹ is 3-14 membered heterocyclyl optionally substituted with R⁴;
Z² is:
(i) absent;
(ii) carbonyl; or
(iii) —C(O)NHCH₂—;
Cy² is:
(i) C₆-C₁₀-aryl;
(ii) 3-14 membered heterocyclyl; or
(iii) 5-14 membered heteroaryl;
Z³ is —X²(CH₂)ₑ—;
Cy³ is 3-14 membered heterocyclyl;
e is an integer selected from 0, 1 and 2;
X² is:
(i) absent; or
(ii) —O—;
R⁴ is C₆-C₁₀-aryl; and
the wavy line indicates the point of attachment to the linker.

4. The compound of formula (I) according to claim 1, or a pharmaceutically acceptable salt thereof, wherein said targeting ligand is of formula (TL), wherein:
R¹ is selected from the group consisting of hydrogen and fluoro;
R² is hydrogen;
R³ is hydroxy;
Z¹ is absent;
Cy¹ is selected from the group consisting of: 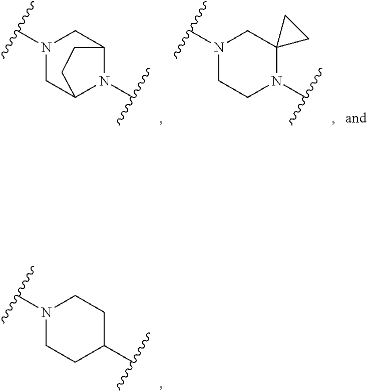 [SMILES: *N1CC2CCC(C1)N2* ; *N1CCN(*)C2(CC2)C1 ; *C1CCN(*)CC1]
optionally substituted with R⁴;
wherein each wavy line indicates the point of attachment to Z² or to the remainder of formula (TL);
Z² is:
(i) absent;
(ii) carbonyl; or
(iii) —C(O)NHCH₂—;
Cy² is
(i) phenyl;
(ii) 3-14 membered heterocyclyl selected from: 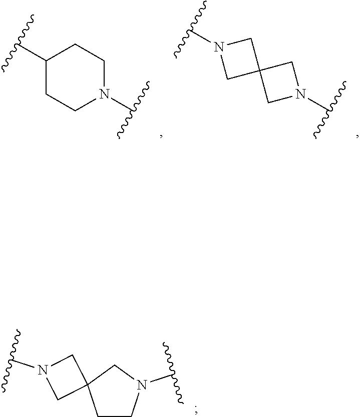 [SMILES: *C1CCN(*)CC1 ; *N1CC2(C1)CN(*)C2 ; *N1CCC2(C1)CN(*)C2]
wherein each wavy line indicates the point of attachment to Z² or Z³; or
(iii) pyrimidinyl;
Z³ is —X²(CH₂)ₑ—;
Cy³ is 3-14 membered heterocyclyl selected from: 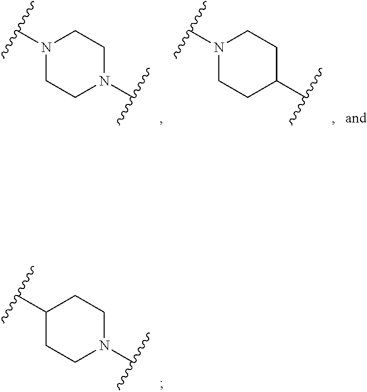 [SMILES: *N1CCN(*)CC1 ; *C1CCN(*)CC1 ; *C1CCN(*)CC1]
wherein each wavy line indicates the point of attachment to Z³ or the linker;
e is an integer selected from 0, 1 and 2;
X² is:
(i) absent; or
(ii) —O—;
R⁴ is phenyl; and
the wavy line indicates the point of attachment to the linker.

5. The compound of formula (I) according to claim 1, or a pharmaceutically acceptable salt thereof, wherein said linker is a covalent bond or is selected from the group consisting of formulae L-1 to L-23, wherein:
X³ and X⁴ are independently selected from the group consisting of CH and N;
R¹² and R¹³ are independently selected from the group consisting of hydrogen and C₁-C₆-alkyl; or
R¹² and R¹³, taken together with the carbon atom to which they are attached, form a C₃-C₁₀-cycloalkyl ring;
R¹⁴ is hydrogen or C₁-C₆-alkyl;
R¹⁵ is hydrogen;
R¹⁶ is C₁-C₆-alkyl;
R¹⁷ is hydrogen;
f is an integer selected from 1, 2, 5, 6, 7, 8, 9
g is an integer selected from 3, 6, 8, 9, 10, 11, 14
h is 2,
i is an integer selected from 0, 1, 2, 3,
k is 3;
m is 1;
n is an integer selected from 8 and 12;
p is an integer selected from 0, 1, and 8;
q is 7;
r is an integer selected from 0 and 1;
s is 4;
t is 9;
u is 4;
v is 1;
w is 4;
x is an integer selected from 2 and 4;
y is an integer selected from 1 and 3;
z is 1;
aa is an integer selected from 0, 1, and 8;
Y¹ is —O— or —NH—;
Y² is —O—, —NH—, —C₁-C₆-alkyldiyl- or —NH—C₁-C₆-alkyldiyl-;
Y³ is absent, —O—C₁-C₆-alkyldiyl- or carbonyl;
Y⁴ is —O—, —NH—, —N(C₁-C₆-alkyl)- or —C₁-C₆-alkyldiyl-;
Y⁵ is absent or carbonyl;
Y⁶ is absent, carbonyl, —O—, —NHC(O)—, —C(O)—N(C₁-C₆-alkyl)- or —C(O)NH—;
Y⁷ is absent or —C₁-C₆-alkyldiyl-;
Y⁸ is absent or —O—;
Y⁹ is —NH—; and
each occurrence of a wavy line indicates the point of attachment of the linker to the targeting ligand or to the degron.

6. The compound of formula (I) according to claim 1, or a pharmaceutically acceptable salt thereof, wherein said linker is a covalent bond or is selected from the group consisting of formulae L-4, L-8, L-13, and L-23, wherein:
i is an integer selected from 0, 2, and 3;
p is an integer selected from 0 and 1;
aa is 0;
Y⁵ is absent;
Y⁶ is absent, carbonyl, —O— or —C(O)—N(C₁-C₆-alkyl)-;
Y⁸ is absent or —O—; and
each occurrence of a wavy line indicates the point of attachment of the linker to the targeting ligand or to the degron.

7. The compound of formula (I) according to claim 1, or a pharmaceutically acceptable salt thereof, wherein said linker is a covalent bond or is selected from the group consisting of formulae L-4, L-8, L-13, and L-23, wherein:
i is an integer selected from 0, 2, and 3;
p is an integer selected from 0 and 1;
aa is 0;
Y⁵ is absent;
Y⁶ is absent, carbonyl, —O— or —C(O)—NCH₃—;
Y⁸ is absent or —O—; and
each occurrence of a wavy line indicates the point of attachment of the linker to the targeting ligand or to the degron.

8. The compound of formula (I) according to claim 1, or a pharmaceutically acceptable salt thereof, wherein said degron is selected from the group consisting of formulae (DG-1) and (DG-2), wherein:
X⁵ is CH or N;
X⁶ is CH₂ or C(O);
R¹⁸ is hydrogen;
Y¹⁰ is a covalent bond, —O— or —NH—; and
the wavy line indicates the point of attachment to the linker.

9. The compound of formula (I) according to claim 1, or a pharmaceutically acceptable salt thereof, wherein said degron is selected from the group consisting of formulae (DG-1) and (DG-2), wherein:
X⁵ is CH;
X⁶ is C(O);
R¹⁸ is hydrogen;
Y¹⁰ is —NH—; and
the wavy line indicates the point of attachment to the linker.

10. The compound of formula (I) according to claim 1, or a pharmaceutically acceptable salt thereof, wherein said targeting ligand is of formula (TL), wherein:
R¹ and R² are each independently selected from the group consisting of hydrogen and halogen;
R³ is selected from the group consisting of amino and hydroxy;
Z¹ is:
(i) absent;
(ii) —O—; or
(iii) —O—C₁-C₆-alkyldiyl-CH(C₆-C₁₀-aryl)-C₁-C₆-alkyldiyl-NHC(O)—;
Cy¹ is 3-14 membered heterocyclyl optionally substituted with R⁴;
Z² is:
(i) absent;
(ii) carbonyl;
(iii) —O(CH₂)ₐ—;
(iv) —C(O)NH(CH₂)_{b}—;
(v) —(CH₂)_{c}NHC(O)(CH₂)_{d}X¹—;
(vi) —O—C₁-C₆-alkyldiyl-C(O)NH—; or
(vii) —CH₂N(C₁-C₆-alkyl)CH₂—;
Cy² is:
(i) absent;
(ii) C₆-C₁₀-aryl optionally substituted with R⁵;
(iii) C₃-C₁₀-cycloalkyl;
(iv) 3-14 membered heterocyclyl; or
(v) 5-14 membered heteroaryl;
Z³ is:
(i) absent;
(ii) —X²(CH₂)ₑ—; or
(iii) —(CH₂)ₑX²;
Cy³ is:
(i) absent;
(ii) C₆-C₁₀-aryl;
(iii) C₃-C₁₀-cycloalkyl; or
(iv) 3-14 membered heterocyclyl;
a is 0, 1 or 2;
b is 0 or 1;
c, d, and e are each independently an integer selected from 0, 1, 2 and 3;
X¹ is:
(i) absent;
(ii) —NH—; or
(iii) —O—;
X² is:
(i) absent;
(ii) carbonyl;
(iii) —O—; or
(iv) —NHC(O)—;
R⁴ is C₆-C₁₀-aryl;
R⁵ is selected from the group consisting of halogen, C₁-C₆-alkyl, and halo-C₁-C₆-alkyl; and
the wavy line indicates the point of attachment to the linker;
wherein said linker is a covalent bond or is selected from the group consisting of formulae L-1 to L-23, wherein:
X³ and X⁴ are independently selected from the group consisting of CH and N;
R¹² and R¹³ are independently selected from the group consisting of hydrogen and C₁-C₆-alkyl; or
R¹² and R¹³, taken together with the carbon atom to which they are attached, form a C₃-C₁₀-cycloalkyl ring;
R¹⁴ is hydrogen or C₁-C₆-alkyl;
R¹⁵ is hydrogen;
R¹⁶ is C₁-C₆-alkyl;
R¹⁷ is hydrogen;
f is an integer selected from 1, 2, 5, 6, 7, 8, 9
g is an integer selected from 3, 6, 8, 9, 10, 11, 14
h is 2,
i is an integer selected from 0, 1, 2, 3,
k is 3;
m is 1;
n is an integer selected from 8 and 12;
p is an integer selected from 0, 1, and 8;
q is 7;
r is an integer selected from 0 and 1;
s is 4;
t is 9;
u is 4;
v is 1;
w is 4;
x is an integer selected from 2 and 4;
y is an integer selected from 1 and 3;
z is 1;
aa is an integer selected from 0, 1, and 8;
Y¹ is —O— or —NH—;
Y² is —O—, —NH—, —C₁-C₆-alkyldiyl- or —NH—C₁-C₆-alkyldiyl-;
Y³ is absent, —O—C₁-C₆-alkyldiyl- or carbonyl;
Y⁴ is —O—, —NH—, —N(C₁-C₆-alkyl)- or —C₁-C₆-alkyldiyl-;
Y⁵ is absent or carbonyl;
Y⁶ is absent, carbonyl, —O—, —NHC(O)—, —C(O)—N(C₁-C₆-alkyl)- or —C(O)NH—;
Y⁷ is absent or —C₁-C₆-alkyldiyl-;
Y⁸ is absent or —O—;
Y⁹ is —NH—; and
each occurrence of a wavy line indicates the point of attachment of the linker to the targeting ligand or to the degron; and
wherein said degron is selected from the group consisting of formulae (DG-1) and (DG-2), wherein:
X⁵ is CH or N;
X⁶ is CH₂ or C(O);
R¹⁸ is hydrogen;
Y¹⁰ is a covalent bond, —O— or —NH—; and
the wavy line indicates the point of attachment to the linker.

11. The compound of formula (I) according to claim 1, or a pharmaceutically acceptable salt thereof, wherein said targeting ligand is of formula (TL), wherein:
R¹ is selected from the group consisting of hydrogen and halogen;
R² is hydrogen;
R³ is hydroxy;
Z¹ is absent;
Cy¹ is 3-14 membered heterocyclyl optionally substituted with R⁴;
Z² is:
(i) absent;
(ii) carbonyl; or
(iii) —C(O)NHCH₂—;
Cy² is:
(i) C₆-C₁₀-aryl;
(ii) 3-14 membered heterocyclyl; or
(iii) 5-14 membered heteroaryl;
Z³ is —X²(CH₂)ₑ—;
Cy³ is 3-14 membered heterocyclyl;
e is an integer selected from 0, 1 and 2;
X² is:
(i) absent; or
(ii) —O—;
R⁴ is C₆-C₁₀-aryl; and
the wavy line indicates the point of attachment to the linker;
wherein said linker is a covalent bond or is selected from the group consisting of formulae L-4, L-8, L-13, and L-23, wherein:
i is an integer selected from 0, 2, and 3;
p is an integer selected from 0 and 1;
aa is 0;
Y⁵ is absent;
Y⁶ is absent, carbonyl, —O— or —C(O)—N(C₁-C₆-alkyl)-;
Y⁸ is absent or —O—; and
each occurrence of a wavy line indicates the point of attachment of the linker to the targeting ligand or to the degron; and
wherein said degron is selected from the group consisting of formulae (DG-1) and (DG-2), wherein:
X⁵ is CH;
X⁶ is C(O);
R¹⁸ is hydrogen;
Y¹⁰ is —NH—; and
the wavy line indicates the point of attachment to the linker.

12. The compound of formula (I) according to claim 1, or a pharmaceutically acceptable salt thereof, wherein said targeting ligand is of formula (TL), wherein:
R¹ is selected from the group consisting of hydrogen and fluoro;
R² is hydrogen;
R³ is hydroxy;
Z¹ is absent;
Cy¹ is selected from the group consisting of: 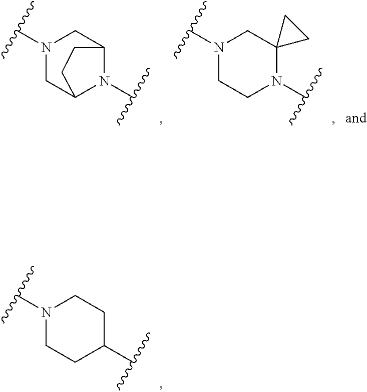 [SMILES: *N1CC2CCC(C1)N2* ; *N1CCN(*)C2(CC2)C1 ; *C1CCN(*)CC1]
optionally substituted with R⁴;
wherein each wavy line indicates the point of attachment to Z² or to the remainder of formula (TL);
Z² is:
(i) absent;
(ii) carbonyl; or
(iii) —C(O)NHCH₂—;
Cy² is
(i) phenyl;
(ii) 3-14 membered heterocyclyl selected from: 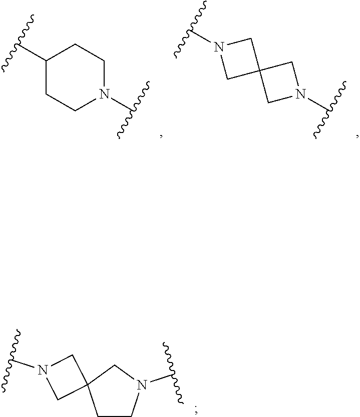 [SMILES: *C1CCN(*)CC1 ; *N1CC2(C1)CN(*)C2 ; *N1CCC2(C1)CN(*)C2]
wherein each wavy line indicates the point of attachment to Z² or Z³; or
(iii) pyrimidinyl;
Z³ is —X²(CH₂)ₑ—;
Cy³ is 3-14 membered heterocyclyl selected from: 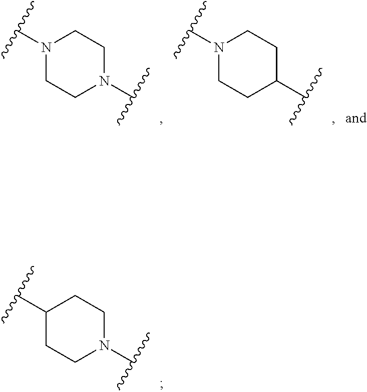 [SMILES: *N1CCN(*)CC1 ; *C1CCN(*)CC1 ; *C1CCN(*)CC1]
wherein each wavy line indicates the point of attachment to Z³ or the linker;
e is an integer selected from 0, 1 and 2;
X² is:
(i) absent; or
(ii) —O—;
R⁴ is phenyl; and
the wavy line indicates the point of attachment to the linker;
wherein said linker is a covalent bond or is selected from the group consisting of formulae L-4, L-8, L-13, and L-23, wherein:
i is an integer selected from 0, 2, and 3;
p is an integer selected from 0 and 1;
aa is 0;
Y⁵ is absent;
Y⁶ is absent, carbonyl, —O— or —C(O)—NCH₃—;
Y⁸ is absent or —O—; and
each occurrence of a wavy line indicates the point of attachment of the linker to the targeting ligand or to the degron; and
wherein said degron is selected from the group consisting of formulae (DG-1) and (DG-2), wherein:
X⁵ is CH;
X⁶ is C(O);
R¹⁸ is hydrogen;
Y¹⁰ is —NH—; and
the wavy line indicates the point of attachment to the linker.

13. The compound of formula (I) according to claim 1, or a pharmaceutically acceptable salt thereof, wherein said compound of formula (I) is selected from Examples 1 to 204.

14. The compound of formula (I) according to claim 1, or a pharmaceutically acceptable salt thereof, wherein said compound of formula (I) is selected from Examples 34, 35, 36, 46, 55, 84, 95, 96, 100, 113, 113, 114, 118, 127, 142, 143, 149, 149, 158, 159, 161, 170, 190, and 191.

15. A therapeutically active substance, comprising the compound of formula (I) according to claim 1, or a pharmaceutically acceptable salt thereof.

16. A pharmaceutical composition, comprising a compound of formula (I) according to claim 1, or a pharmaceutically acceptable salt thereof, and a therapeutically inert carrier.

17. The composition according to claim 16, further comprising an additional therapeutic agent.

18. The composition according to claim 16, wherein the additional therapeutic agent is a chemotherapeutic agent.

19. (canceled)

20. The method according to claim 23, wherein said SMARCA2-mediated disorder is cancer.

21. The method according to claim 20, wherein said cancer is selected from the group consisting of acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, liver cancer, lung cancer, lymphagioendotheliosarcoma, lymphangiosarcoma, lymphoblastic leukemia, lymphoma (Hodgkin's and non-Hodgkin's; Burkitt's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

22. The method according to claim 20, wherein said cancer is selected from the group consisting of hepatocellular cancer, malignancies and hyperproliferative disorders of the colon (e.g. colon cancer), lung cancer, breast cancer, prostate cancer, melanoma, and ovarian cancer.

23. A method of treating SMARCA2-mediated disorders in a subject, comprising administering a compound of formula (I) according to claim 1, or a pharmaceutically acceptable salt thereof, to the subject.

24. (canceled)

25. (canceled)

26. (canceled)
